(19) United States
(12) Patent Application Publication
(10) Pub. No.: US 2022/0275367 A1
(43) Pub. Date: Sep. 1, 2022

(54) COMPOSITIONS AND METHODS FOR TREATING HUNTINGTON'S DISEASE

(71) Applicant: VOYAGER THERAPEUTICS, INC., Cambridge, MA (US)

(72) Inventors: Dinah Wen-Yee Sah, Hopkinton, MA (US); Pengcheng Zhou, Lexington, MA (US); Jeffrey S. Thompson, Stoneham, MA (US); Christina Gamba-Vitalo, Arlington, MA (US); Jenna Carroll Soper, Winchester, MA (US); Steven M. Hersch, Cambridge, MA (US); Todd Carter, Winchester, MA (US); Jacob J. Cardinal, Cambridge, MA (US); Lori B. Karpes, Ashland, MA (US); Bin Liu, Boston, MA (US)

(21) Appl. No.: 17/629,552

(22) PCT Filed: Jul. 24, 2020
(86) PCT No.: PCT/US2020/043366

§ 371 Date: Jan. 24, 2022

Related U.S. Application Data
(60) Provisional application No. 63/021,861, filed on May 8, 2020.
(60) Provisional application No. 62/924,400, filed on Oct. 22, 2019.
(60) Provisional application No. 62/913,407, filed on Oct. 10, 2019.
(60) Provisional application No. 62/878,054, filed on Jul. 24, 2019.

(51) Int. Cl. C12N 15/113; A61K 35/76; A61K 47/02; A61K 47/26; A61K 47/10; A61P 25/28

(52) U.S. Cl. CPC C12N 15/113; A61K 35/76; A61K 47/02; A61K 47/26; A61K 47/10; A61P 25/28; C12N 2310/14

(57) ABSTRACT

The present disclosure relates to pharmaceutical compositions of adeno-associated viral (AAV) particles encoding siRNA molecules and methods for treating Huntington's Disease (HD).

### CROSS REFERENCE TO RELATED APPLICATIONS

This application claims the benefit of U.S. Provisional Patent Application No. 62/878,054, filed Jul. 24, 2019, entitled COMPOSITIONS AND METHODS FOR TREATING HUNTINGTON'S DISEASE; U.S. Provisional Patent Application No. 62/913,407, filed Oct. 10, 2019, entitled COMPOSITIONS AND METHODS OF TREATING HUNTINGTON'S DISEASE; U.S, Provisional Patent Application No. 62/924,400, filed Oct. 22, 2019, entitled COMPOSITIONS AND METHODS OF TREATING HUNTINGTON'S DISEASE; U.S. Provisional Patent Application No. 63/021,861, filed May 8, 2020, entitled COMPOSITIONS AND METHODS OF TREATING HUNTINGTON'S DISEASE; the contents of each of which are incorporated herein by reference in their entirety.

### SEQUENCE LISTING

The present application is being filed along with a Sequence Listing in electronic format. The Sequence Listing file, entitled 2057_1086PCT_SL.txt, was created on Jul. 24, 2020, and is 6,725,315 bytes in size. The information in electronic format of the Sequence Listing is incorporated herein by reference in its entirety.

### FIELD OF THE DISCLOSURE

Provided herein are compositions, methods and processes for the design, preparation, manufacture, use and/or formulation of AAV particles comprising modulatory polynucleotides, e.g., polynucleotides encoding small interfering RNA (siRNA) molecules which target the Huntingtin (HTT) gene (e.g., the wild-type or the mutated CAG-expanded. HTT gene). Targeting of the mutated HTT gene may interfere with the HTT gene expression and the resultant HTT protein production. The AAV particles comprising modulatory polynucleotides encoding the siRNA molecules may be inserted into recombinant adeno-associated virus (AAV) vectors. Methods for using the AAV particles to inhibit the HTT gene expression in a subject with a neurodegenerative disease (e.g., Huntington's Disease (HD)) are also disclosed.

### BACKGROUND

Huntington's Disease (HD) is a monogenic fatal neurodegenerative disease characterized by progressive chorea, neuropsychiatric and cognitive dysfunction. Huntington's Disease is known to be caused by an autosomal dominant triplet (CAG) repeat expansion which encodes poly-glutamine in the N-terminus of the huntingtin (HTT) protein. This repeat expansion results in a toxic gain of function of HTT and ultimately leads to striatal neurodegeneration which progresses to widespread brain atrophy. Symptoms typically appear between the ages of 35-44 and life expectancy subsequent to onset is 10-25 years. Interestingly, the length of the HTT expansion correlates with both age of onset and rate of disease progression, with longer expansions linked to greater severity of disease. In a small percentage of the HD population (˜6%), disease onset occurs from 2-20 years of age with appearance of an akinetic-rigid syndrome. These cases tend to progress faster than those of the later onset variety and have been classified as juvenile or Westphal variant HD. It is estimated that approximately 35,000-70,000 patients are currently suffering from HD in the US and Europe. Currently, only symptomatic relief and supportive therapies are available for treatment of HD, with a cure yet to be identified. Ultimately, individuals with HD succumb to other diseases (e.g., pneumonia, heart failure), choking, suffocation or other complications such as physical injury from falls.

The mechanisms by which CAG-expanded HTT results in neurotoxicity are not well understood. Huntingtin protein is expressed in all cells, though its concentration is highest in the brain. The normal function of HTT is unknown, but in the brains of HD patients, HTT aggregates into abnormal nuclear inclusions. It is now believed that it is this process of misfolding and aggregating along with the associated protein intermediates (i.e. the soluble species and toxic N-terminal fragments) that result in neurotoxicity.

Studies in animal models of HD have suggested that phenotypic reversal is feasible, for example, subsequent to gene shut off in regulated-expression models. Further, animal models in which silencing of HTT was tested, demonstrated promising results with the therapy being both well tolerated and showing potential therapeutic benefit. These findings indicate that HTT silencing may serve as a potential therapeutic target for treatment of HD.

The adeno-associated virus (AAV) is a member of the parvovirus family and has emerged as an attractive vector for gene therapy in large part because this virus is apparently non-pathogenic; in fact, AAV has not been associated with any human disease. Further appeal is due to its ability to transduce dividing and non-dividing cells (including efficient transduction of neurons), diminished proinflammatory and immune responses in humans, inability to autonomously replicate without a helper virus (AAV is a helper-dependent DNA parvovirus which belongs to the genus Dependovirus), and its long-term gene expression. Although over 10 recombinant AAV serotypes (rAAV) have been engineered into vectors, rAAV2 is the most frequently employed serotype for gene therapy trials. Additional rAAV serotypes have been developed and tested in animal models that are more efficient at neuronal transduction.

The present disclosure develops an AAV particle comprising modulatory polynucleotides encoding novel double stranded RNA (dsRNA) constructs and siRNA constructs and methods of their design, to inhibit or prevent the expression of CAG-expanded HTT in HD patients for treatment of the disease. The present disclosure further discloses formulations, dosing and administration of the AAV particle comprising modulatory polynucleotides (e.g, siRNA) targeting HTT mRNA for the treatment of HD.

### SUMMARY

Described herein are compositions, methods, processes, kits and/or devices for the administration of AAV particles comprising modulatory polynucleotides encoding siRNA molecules for the treatment, prophylaxis, palliation and/or amelioration of Huntington's Disease (HD) related symptoms and disorders.

The present disclosure provides pharmaceutical compositions for use in the treatment of Huntington's Disease (HD) comprising AAV particles, wherein at least one of the AAV particles comprises an AAV viral genome comprising modulatory polynucleotides (e.g., siRNA) targeting HTT mRNA in a pharmaceutically acceptable formulation.

In some embodiments, the concentration of the AAV viral genome in the pharmaceutical composition is from 1×10¹¹ to 1×10¹² VG/mL. In some embodiments the concentration of the AAV viral genome is from 1×10¹¹ to 9×10¹¹ VG/mL. In some embodiments, the concentration of the AAV viral genome is from 1.2×10¹¹ to 6×10¹¹ VG/mL. In some embodiments, the concentration of the AAV viral genome is from 1.8×10¹¹to 6×10¹¹ VG/mL. In some embodiments, the concentration of the AAV viral genome is from 5×10¹¹ to 8×10¹¹ VG/mL.

In some embodiments, the AAV particle comprises an AAV viral genome comprising a polynucleotide sequence selected from the group consisting of SEQ ID NO: 1352-1379. 1388, and 1426-1438 or variants having at least 95% identity thereof. In some embodiments, the polynucleotide sequence comprises SEQ ID NO: 1352.

In some embodiments, the AAV particle may comprise an AAV capsid comprising a capsid serotype such as, but not limited to, any of the capsid serotypes listed in Table 1. In some embodiments, the AAV particle capsid serotype may be an AAV1. serotype.

In some embodiments, the pharmaceutically acceptable formulation is an aqueous solution comprising: a) one or more salts such as, but not limited to, sodium chloride, potassium chloride, and potassium phosphate, or combination thereof; b) at least one disaccharide such as, but not limited to, sucrose; and c) a buffering agent that may be selected from Tris HCl, Tris base, sodium phosphate, potassium phosphate, histidine, boric acid, citric acid, glycine, HEPES (4-(2-hydroxyethyl)-1-piperazineethanesulfonic acid), and MOPS (3-(N-morpholino)propanesulfonic acid).

In some embodiments, the concentration of sodium chloride comprising the pharmaceutically acceptable formulation may be from 85 to 110 mM. In some embodiments, the pharmaceutically acceptable formulation may comprise sodium chloride at a concentration of 95 mM.

In some embodiments, the concentration of potassium chloride comprising the pharmaceutically acceptable formulation may be from 1 to 3 mM. In some embodiments, the pharmaceutically acceptable formulation may comprise potassium chloride at a concentration of 1.5 mM.

In some embodiments. the concentration of potassium phosphate comprising the pharmaceutically acceptable formulation may be from 1 to 3 mM. In some embodiments, the pharmaceutically acceptable formulation may comprise potassium phosphate at a concentration of 1.5 mM.

In some embodiments, the sucrose comprising the pharmaceutically acceptable formulation may be at a concentration that is 5 to 9% by weight relative to the total volume of the pharmaceutically acceptable formulation. In some embodiments, the sucrose may be at a concentration that is 7% by weight relative to the total volume of the pharmaceutically acceptable formulation

In some embodiments. the concentration of buffering agent comprising the pharmaceutically acceptable formulation may be from 10 mM.

In some embodiments, the pharmaceutical composition may be buffered to a pH from 7.2 to 8.2 at 5° C. In some embodiments, the buffering agent may be sodium phosphate and the formulation may be buffered to a pH from 7.2 to 7.6 at 5° C. In some embodiments, the buffering agent may be Tris base that may be adjusted with hydrochloric acid to a pH from 7.3 to 7.7 at 5° C.

In some embodiments, the pharmaceutical composition may further comprise a surfactant. In some embodiments, the surfactant may be Poloxamer 188. The concentration of Poloxamer 188 may be from 0.01% by weight (mg/L) relative to the total volume of the pharmaceutically acceptable formulation.

In some embodiments, the pharmaceutically acceptable formulation may have an osmolality of 400 to 480 mOsm/kg.

Further provided herein are methods of treating Huntington's Disease in a patient in need thereof, by administering to the patient a therapeutically effective amount of the pharmaceutical composition described herein. In some embodiments, the pharmaceutical composition may be administered via infusion into the striatum of the patient. The infusion may be bilaterally or unilaterally infused into the striatum of the patient. In some embodiments, the pharmaceutical composition may be administered via infusion into the putamen and thalamus of the patient. The infusion may be independently bilateral or unilateral into the putamen and thalamus of the patient. The pharmaceutical composition may be administered using magnetic resonance imaging (MRI)-guided convection enhanced delivery (CED).

In some embodiments, the volume of the pharmaceutical composition administered to the striatum may be 15 μL/hemisphere or less. In some embodiments, the volume of the pharmaceutical composition administered to the striatum may be from 5-10 μL/hemisphere.

In some embodiments, the dose administered to the striatum may be between 2×10⁹ to 3×10¹¹VG/hemisphere.

In some embodiments, the volume of the pharmaceutical composition administered to the putamen may be 1500 μL/hemisphere or less. In some embodiments, the volume of the pharmaceutical composition administered to the putamen may be from 100-1500 μL/hemisphere.

In some embodiments, the dose administered to the putamen may be between 1×10¹⁰ to 4×10¹³ VG/hemisphere.

In some embodiments, the volume of the pharmaceutical composition administered to the thalamus may be 2500 μL/hemisphere or less. The volume of the pharmaceutical composition administered to the thalamus may be from 150-2500 μL/hemisphere.

In some embodiments, the dose administered to the thalamus may be between 4×10¹¹ to 6.8×10^{13 VG/hemisphere.}

In some embodiments, the total dose administered to the patient may be between 8×10⁹ to 2×10¹⁴ VG.

In some embodiments, the methods described herein inhibit or suppress the expression of the Huntingtin (HTT) gene product (RNA or protein) in a tissue such as, but not limited to, the striatum, putamen, caudate, thalamus, cerebral cortex, primary motor cortex, primary somatosensory cortex, temporal cortex, and combinations thereof, of a patient comprising administering a therapeutically effective amount of the pharmaceutical compositions disclosed herein.

In some embodiments, the expression of the HTT gene product (RNA or protein) may be reduced by at least 30%. In some embodiments, expression of the HTT gene product (RNA or protein) may be reduced by 40-70%. In some embodiments, expression of the HTT gene product (RNA or protein) may be reduced by 50-80%.

In some embodiments, the expression of the HTT gene product (RNA or protein) is inhibited or suppressed in the putamen and is measured in one or more medium spiny neurons in the putamen. In some embodiments, the expression of the HTT gene product (RNA or protein) inhibited or suppressed in the putamen and is measured in one or more astrocytes in the putamen.

In some embodiments, the expression of the HTT gene product (RNA or protein) is inhibited or suppressed in pyramidal neurons of each of the primary motor cortex, primary somatosensory cortex, and the temporal cortex. In some embodiments, the expression of the HTT gene product (RNA or protein) may reduced by at least 20% in the cerebral cortex.

In some embodiments, the expression of the HTT gene product (RNA or protein) is inhibited or suppressed in both the striatum and the cerebral cortex of the patient.

In some embodiments, the HTT gene product is the HTT protein and the HTT protein expression is inhibited or suppressed in the striatum, putamen, caudate and/or thalamus of the patient.

In certain embodiments, the level of the HTT protein may be reduced by at least 10% in the putamen. In certain other embodiments, the level of the HTT protein may be reduced by 15-65% in the putamen.

In certain embodiments, the level of the HTT protein may be reduced by at least 5% in the caudate. In certain other embodiments, the level of the HTT protein may be reduced by 5-50% in the caudate.

In certain embodiments, the level of the HTT protein may be reduced by at least 10% in the thalamus. In certain other embodiments, the level of the HTT protein is reduced by 15-80% in the thalamus.

In some embodiments, the level of the HTT protein is reduced in both the striatum and the thalamus of the patient.

### BRIEF DESCRIPTION OF THE DRAWINGS

The foregoing and other objects, features and advantages will be apparent from the following description of particular embodiments described herein, as illustrated in the accompanying drawings. The drawings are not necessarily to scale, emphasis instead being placed upon illustrating the principles of various embodiments described herein.

FIG. 1 is a schematic of a viral genome of the disclosure.

FIG. 2 is a schematic of a viral genome of the disclosure.

FIG. 3 is a schematic of a viral genome of the disclosure.

FIG. 4 is a schematic of a viral genome of the disclosure.

FIG. 5 is a schematic of a viral genome of the disclosure.

FIG. 6 is a schematic of a viral genome of the disclosure.

FIG. 7 is a schematic of a viral genome of the disclosure,

FIG. 8 is a schematic of a viral genome of the disclosure.

FIG. 9 is a schematic of a viral genome of the disclosure.

FIG. 10A are panels of graphs showing HTT mRNA knockdown and vector genome levels in tissue punches collected from the left non-human primate (NHP) putamen.

FIG. 10B are panels of graphs showing HTT mRNA knockdown and vector genome levels in tissue punches collected from right non-human primate (NHP) putamen.

FIG. 10C are panels of graphs showing HTT mRNA knockdown and vector genome levels in all tissue punches collected from non-human primate (NHP) putamen.

FIG. 11A are panels of graphs showing HTT mRNA knockdown and vector genome levels in tissue punches collected from left and right NHP caudate, hCN-1.

FIG. 11B are panels of graphs showing HTT mRNA knockdown and vector genome levels in tissue punches collected from left and right NHP caudate, hCN-2.

FIG. 11C are panels of graphs showing HTT mRNA knockdown and vector genome levels in tissue punches collected from left and right NHP caudate, combined hCN-1 and hCN-2.

FIG. 12A are panels of graphs showing HTT mRNA knockdown and vector genome levels in tissue punches collected from the left NHP motor cortex (mCTX).

FIG. 12B are panels of graphs showing HTT mRNA knockdown and vector genome levels in tissue punches collected from the right NHP motor cortex (mCTX).

FIG. 12C are panels of graphs showing HTT mRNA knockdown and vector genome levels in all tissue punches collected from NUT motor cortex (mCTX).

FIG. 13A are panels of graphs showing HTT mRNA knockdown and vector genome levels in tissue punches collected from the left NHP somatosensory cortex (ssCTX).

FIG. 13B are panels of graphs showing HTT mRNA knockdown and vector genome levels in tissue punches collected from the right NHP somatosensory cortex (ssCTX).

FIG. 13C are panels of graphs showing HTT mRNA knockdown and vector genome levels in all tissue punches collected from the right NHP somatosensory cortex (ssCTX).

FIG. 14A are panels of graphs showing HTT mRNA knockdown and vector genome levels in tissue punches collected from the left NHP temporal cortex (tCTX).

FIG. 14B are panels of graphs showing HTT mRNA knockdown and vector genome levels in tissue punches collected from the right NHP temporal cortex (tCTX).

FIG. 14C are panels of graphs showing HTT mRNA knockdown and vector genome levels in all tissue punches collected from the NHP temporal cortex (tCTX).

FIG. 15A is a graph showing HTT mRNA knockdown in laser captured cortical pyramidal neurons from NHP cortex.

FIG. 15B is a graph showing HTT mRNA knockdown and vector genome levels in laser captured cortical pyramidal neurons from NHP cortex.

FIG. 16A shows a correlation of HTT mRNA knockdown versus vector genome levels in tissue punches taken from the putamen.

FIG. 16B shows a correlation of vector genome versus AAV1VOYHT1. miRNA levels in tissue punches taken from the putamen.

FIG. 16C shows a correlation of AAV1-VOYHT1 miRNA versus HTT mRNA levels in tissue punches taken from the putamen.

FIG. 17A shows a correlation of HTT mRNA knockdown versus vector genome levels in tissue punches taken from the caudate,

FIG. 17B shows a correlation of vector genome versus AAV1-VOYHT1 miRNA levels in tissue punches taken from the caudate.

FIG. 17C shows a correlation of AAV1-VOYHT1 miRNA versus HTT mRNA levels in tissue punches taken from the caudate.

FIG. 18 shows a correlation of HTT mRNA knockdown versus vector genome levels in tissue punches taken from the thalamus.

FIG. 19 shows a linear correlation of relative remaining NHP HTT protein and mRNA levels in the putamen, caudate and thalamus.

The details of one or more embodiments of the disclosure are set forth in the accompanying description below. Although any materials and methods similar or equivalent to those described herein can be used in the practice or testing of the present disclosure, the preferred materials and methods are now described. Other features, objects and advantages of this disclosure will be apparent from the description. In the description, the singular forms also include the plural unless the context clearly dictates otherwise. Unless defined otherwise, all technical and scientific terms used herein have the same meaning as commonly understood by one of ordinary skill in the art to which this disclosure belongs. In the case of conflict, the present description will control.

### DETAILED DESCRIPTION

### I. Compositions

According to the present disclosure, compositions for delivering modulatory polynucleotides and/or modulatory polynucleotide-based compositions by adeno-associated viruses (AAVs) are provided, AAV particles described herein may be provided via any of several routes of administration, to a cell, tissue, organ, or organism, in vivo, ex vivo or in vitro.

As used herein, an “AAV particle” is a virus which includes a capsid and a viral genome with at least one payload region and at least one ITR region. AAV particles of the present disclosure may be produced recombinantly and may be based on adeno-associated virus (AAV) parent or reference sequences. AAV particles may be derived from any serotype, described herein or known in the art, including combinations of serotypes (i.e., “pseudotyped” AAV) or from various genomes (e.g., single stranded or self-complementary). In addition, the AAV particle may be replication defective and/or targeted.

As used herein, “viral genome” or “vector genome” or “viral vector” refers to the nucleic acid sequence(s) encapsulated in an AAV particle. Viral genomes comprise at least one payload region encoding polypeptides or fragments thereof.

As used herein, a “payload” or “payload region” is any nucleic acid molecule which encodes one or more polypeptides of this disclosure. At a minimum, a payload region comprises nucleic acid sequences that encode a sense and antisense sequence, an siRNA-based composition, or a fragment thereof, but may also optionally comprise one or more functional or regulatory elements to facilitate transcriptional expression and/or polypeptide translation.

The nucleic acid sequences and polypeptides disclosed herein may be engineered to contain modular elements and/or sequence motifs assembled to enable expression of the modulatory polynucleotides and/or modulatory polynucleotide-based compositions. In some embodiments, the nucleic acid sequence comprising the payload region may comprise one or more of a promoter region, an intron, a Kozak sequence, an enhancer, or a polyadenylation sequence. Payload regions disclosed herein typically encode at least one sense and antisense sequence, an siRNA-based composition, or fragments of the foregoing in combination with each other or in combination with other polypeptide moieties.

The payload regions within the viral genome of an AAV particle of the disclosure may he delivered to one or more target cells, tissues, organs, or organisms.

### Adeno-Associated Viruses (AAVs) and AAV Particles

Viruses of the Parvoviridae family are small non-enveloped icosahedral capsid viruses characterized by a single stranded DNA genome. Parvoviridae family viruses consist of two subfamilies: Parvovirinae, which infect vertebrates, and Densovirinae, which infect invertebrates. Due to its relatively simple structure, easily manipulated using standard molecular biology techniques, this virus family is useful as a biological tool. The genome of the virus may he modified to contain a minimum of components for the assembly of a functional recombinant virus, or viral particle, which is loaded with or engineered to express or deliver a desired payload, which may be delivered to a target cell, tissue, organ, or organism.

The parvoviruses and other members of the Parvoviridae family are generally described in Kenneth I. Berns, “Parvoviridae: The Viruses and Their Replication,” Chapter 69 in FIELDS VIROLOGY (3d Ed, 1996), the contents of which are incorporated by reference in their entirety:

The Parvoviridae family comprises the Dependovirus genus which includes adeno-associated viruses (AAV) capable of replication in vertebrate hosts including, but not limited to, human, primate, porcine, bovine, canine, equine, and ovine species.

The AAV viral genome (VG) is a linear, single-stranded DNA (ssDNA) molecule or self-complementary (scAAV) approximately 5,000 nucleotides (nt) in length. The AAV viral genome can comprise a payload region and at least one inverted terminal repeat (ITR) or ITR region. ITRs traditionally flank the coding nucleotide sequences for the non-structural proteins (encoded by Rep genes) and the structural proteins (encoded by capsid genes or Cap genes). While not wishing to be bound by theory, an AAV viral genome typically comprises two ITR sequences, The AAV viral genome comprises a characteristic T-shaped hairpin structure defined by the self-complementary terminal 145 nt of the 5′ and 3′ ends of the ssDNA which form an energetically stable double stranded region. The double stranded hairpin structures comprise multiple functions including, but not limited to, acting as an origin for DNA replication by functioning as primers for the endogenous DNA polymerase complex of the host viral replication cell.

In addition to the encoded heterologous payload, AAV vectors may comprise the viral genome, in whole or in part, of any naturally occurring and/or recombinant AAV serotype nucleotide sequence or variant. AAV variants may have sequences of significant homology at the nucleic acid (genome or capsid) and amino acid levels (capsids), to produce constructs which are generally physical and functional equivalents, replicate by similar mechanisms, and assemble by similar mechanisms. Chiorini et al., J. Vir, 71: 6823-33(1997); Srivastava et al., J. Vir. 45:555-64 (1983); Chiorini et al., J. Vir. 73:1309-1319 (1999); Rutledge et al., J. Vir. 72:309-319 (1998); and \Vu et al., J. Vir. 74: 8635-47 (2000), the contents of each of which are incorporated herein by reference in their entirety.

In some embodiments, AAV particles of the present disclosure are recombinant AAV vectors which are replication defective, lacking sequences encoding functional Rep and Cap proteins within their viral genome. These defective AAV vectors may lack most or all parental coding sequences and essentially carry only one or two AAV ITR sequences and the nucleic acid of interest for delivery to a cell, a tissue, an organ, or an organism.

In some embodiments, the viral genome of the AAV particles of the present disclosure comprise at least one control element which provides for the replication, transcription and translation of a coding sequence encoded therein. Not all the control elements need always be present as long as the coding sequence is capable of being replicated, transcribed, and/or translated in an appropriate host cell. Non-limiting examples of expression control elements include sequences for transcription initiation and/or termination, promoter and/or enhancer sequences, efficient RNA processing signals such as splicing and polyadenylation signals, sequences that stabilize cytoplasmic mRNA, sequences that enhance translation efficacy (e.g., Kozak consensus sequence), sequences that enhance protein stability, and/or sequences that enhance protein processing and/or secretion.

According to the present disclosure, AAV particles for use in therapeutics and/or diagnostics comprise a virus that has been distilled or reduced to the minimum components necessary for transduction of a nucleic acid payload or cargo of interest. In this manner, AAV particles are engineered as vehicles for specific delivery while lacking the deleterious replication and/or integration features found in wild-type viruses.

AAV vectors of the present disclosure may be produced recombinantly and may be based on adeno-associated virus (AAV) parent or reference sequences. As used herein, a “vector” is any molecule or moiety which transports, transduces, or otherwise acts as a carrier of a heterologous molecule such as the nucleic acids described herein.

In addition to single stranded AAV viral genomes (e.g., ssAAVs), the present disclosure also provides for self-complementary AAV (scAAVs) viral genomes, scAAV viral genomes contain DNA strands which anneal together to form double stranded DNA. By skipping second strand synthesis, scAAVs allow for rapid expression in the cell.

in some embodiments, the AAV particle of the present disclosure is an scAAV.

In some embodiments, the AAV particle of the present disclosure is an ssAAV.

Methods for producing and/or modifying AAV particles are disclosed in the art such as pseudotyped AAV vectors (PCT Patent Publication Nos. WO200028004; WO200123001; WO2004112727; WO 2005005610 and WO 2005072364, the content of each of which is incorporated herein by reference in its entirety).

AAV particles may be modified to enhance the efficiency of delivery. Such modified AAV particles can be packaged efficiently and be used to successfully infect the target cells at high frequency and with minimal toxicity. In some embodiments the capsids of the AAV particles are engineered according to the methods described in US Publication Number US 20130195801, the contents of which are incorporated herein by reference in their entirety.

In some embodiments, the AAV particles comprising a payload region encoding the polypeptides described herein may be introduced into mammalian cells.

### AAV Serotypes

AAV particles of the present disclosure may comprise or be derived from any natural. or recombinant AAV serotype. According to the present disclosure, the AAV particles may utilize or be based on a serotype selected from any of the following AAV1, AAA/2, AAV2G9, AAV3, AAV3a, AAV3b, AAV3-3, AAV4, AAV4-4, AAVS, AAV6, AAV6.1, AAV6.2, AAV6.1.2, AAV7, AAV7.2, AAV8, AAV9, AAV9.11, AAV9.13, AAV9.16, AAV9.24, AAA/9.45, AAA/9.47, AAV9.61, AM/9.68, AAV9.84, AAV9.9, AAV 10, AAV 11, AAV12, AAV1.6.3-AAV24.1-NAV27,3, AAV42.12, A AV42-1b-NAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAA/43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1.-8/rh.49, AAV2-15/rh.,62, AAV2-3/rh.61, AAV2-4/rh.50, AAV2-5/rh.51, AAV3.1/hu.6, AAV3.12/hu.9, AAV3-9/rh.52, AAV3-11/rh.53, AAA/4-8/r11.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.51bb,2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAA/33.12/hu.17, AAV33.4/hu.15, AAV33.8/hu.16, AAA/52/hu.19, AAV52.1/hu.20, AAV58.2/hu.25, AAVA3.3, AAVA3.4, AAVA3.5, AAVA3.7, AAVC1, AAVC2, AAVC5, AAV-DJ, AAV-DJ8, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVLK03, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.10, AAVrh.12, AAVdt.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74, AAVrh8R, AAVrh8R A586R mutant, AAVrh8R R533A mutant, AAAV, BAAV, caprine AAV, bovine AAV, ovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK.15, AAV-LK.16, AAV-LK17, AAV-LK18, AAV-LK19, AAV-PAEC2, AAV-PAEC4, AAV-PAEC6, AAV-PAEC7, AAV-PAEC8, ANV-PAEC11, AAV-PAEC12, AAV-2-pre-miRNA-101, AAV-8h, AAV-8b, AAV-h, AAV-b, AAV SM 10-2, AAV Shuffle 100-1 -AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPENN AAV 10, Japanese AAV 10 serotypes, AAV CBr-7.1, AAV CBr-7.10, AAV CBr-7.2, AAV CBr-7.3, AAV CBr-7.4, AAV CBr-7.5, AAV CBr-7.7, AAV CBr-7.8, AAV CBr-B7.3, AAV CBr-B7.4, AAV CBr-E1, AAV CBr-E2, AAV CBr-E3, AAV CBr-E4, AAV CBr-E5, AAV CBr-e5, AAV CBr-E6, AAV CBr-E7, AAV CBr-E8, AAV CHt-1, AAV CHt-2, AAV CHt-3, AAV CHt-6.1, AAV CHt-6.10, AAV CHt-6.5, AAV CHt-6.6, AAV CHt-6.7, AAV CHt-6.8,AAV CHt-P1, AAV CHt-P2, AAV CHt-P5, AAV CHt-P6, AAV CHt-P8, AAV CHt-P9, AAV CKd-1, AAV CKd-10, AAV CKd-2, AAV CKd-3, AAV CKd-4, AAV CKd-6, AAV CKd-7, AAV CKd-8, AAV CKd-B1, AAV CKd-B2, AAV CKd-B3, AAV CKd-B4, AAV CKd-B5, AAV CKd-B6, AAV CKd-B7, AAV CKd-B8, AAV CKd-H1, AAV CKd-H2, AAV CKd-H3,NAV CKd-H4, AAV CKd-H5, AAV CKd-H6, AAV CKd-N3, AAV CKd-N4, AAV CKd-N9, AAV CLg-F1, AAV CLg-F2, AAV CLg-F3, AAV CLg;-F4, AAV CLg-F5, AAV CLg-F6, AAV CLg-F7, AAV CLg-F8, AAV CLv-1, AAV CLv1-1, AAV Clv1-10, AAV CLv1-2, AAV CLv-12, AAV CLv1-3, AAV CLv-13, AAV CLv1-4, AAV Clv1-7, AAV Clv1-8, AAV Clv1-9, AAV CLv-2 AAV CLv-3, AAV CLv-4, AAV CLv-6,NAV CLv-8, AAV CLv-D1, AAV CLv-D2,NAV AAV CLv-D4, AAV CLv-D5, AAV CLv-D6, AAV CLv-D7, AAV CLv-D8, AAV CLv-E1, AAV CLv-K1, AAV CLv-K3, AAV CLv-K6, AAV CLv-L4, AAV CLv-L5, AAV CLv-L6, AAV CLv-M1, AAV CLv-M11, AAV CLv-M2, AAV CLv-M5, AAV CLv-M6, AAV CLv-M7, AAV CLv-M8, AAV CLv-M9, AAV CLv-R2, AAV CLv-R3, AAV CLv-R4, AAV CLv-R5, AAV CLv-R6, AAV CLv-R7, AAV CLv-R8, AAV CLv-R9, AAV CSp-1, AAV CSp-10, AAV CSp-11, AAV CSp-2, AAV CSp-3, AAV CSp-4, AAV CSp-6, AAV CSp-7, AAV CSp-8, AAV CSp-8.10, AAV CSp-8.2, AAV CSp-8.4, AAV CSp-8.5,NAV CSp-8.6, AAV CSp-8.7, AAV CSp-8.8, AAV CSp-8.9, AAV CSp-9, AAV.hu.48R3, AAV.VR-355, AAV3B, AAV4, AAV5, AAVF1/HSC1, AAVF11/HSC11, AAVF12/FISC12, AAVF13/HSC13, AAVF14/HSC14, AAVF15/HSC15, AAVF16/HSC16, AAVF17/HSC17, AAVF2/HSC2, AAVF3/HSC3, AAVF4/HSC4, AAVF5/HSC5, AAVF6/HSC6, AAVF7/HSC7, AAVF8/HSC8, AAVF9/HSC9, AAV-PHP.B (PHP.B), AAV-PHP.A (PHP.A), G2B-26, G2B-13, TH1.1-32, TH1.1-35, AAVPHP.B2, AAVPHP.B3, AAVPHP.N/PHP.B-DGT, AAVPHP.B-EST, AAVPHP.B-GGT, AAVPHP.B-ATP, AAVPHP.B-ATT-T, AAVPHP.B-DGT-T, AAVPHP.B-GGT-T, AAVPHP.B-SGS, AAVPHP.B-AQP, AAVPHP.B-QQP, AAVPHP.B-SNP(3), AAVPHP.B-SNP, AAVPHP.B-QGT, AAVPHIP.B-NQT, AAVPHP.B-EGS, AAVPHP.B-SGN, AAVPHP.B-EGT, AAVPHP.B-DST, AAVPHP.B-DST, AAVPHP.B-STP, AAVPHP.B-PQP, AAVPHP.B-SQP, AAVPHP.B-QLP, AAVPHP.B-TMP, AAVPHP.B-TTP, AAVPHP.S/G2A12, AAVG2A15/G2A3, AAVG2B4, and/or AAVG2B5 and variants thereof.

In some embodiments, the AAV serotype may be, or have, a modification as described in United States Publication No. US 20160361439, the contents of which are herein incorporated. by reference in their entirety, such as but not limited to, Y252F, Y272F, Y444F, Y500F, Y700F, Y704F, Y730F, Y275F, Y281F, Y508F, Y576F, Y612G, Y673F, and Y720F of the wild-type AAV1, AAV2, AAV3, AAV4, AAV5, AAV6, AAV7, AAV8, AAV9, AAV10, AAV11, AAV12, and hybrids thereof.

In some embodiments, the AAV serotype may be, or have, a mutation as described in U.S. Pat. No. 9,546,112, the contents of which are herein incorporated by reference in their entirety, such as, but not limited to, at least two, but not all the F129L, D418E, K531E, L584F, V598A and H642N mutations in the sequence of AAV6 (SEQ ID NO:4 of U.S. Pat. No. 9,546,112), AAV1 (SEQ ID NO:6 of U.S. Pat. No. 9,546,112), AAV2, AAV3, AAV5, AAV7, AAV9, AAV10 or AAV11 or derivatives thereof. In yet another embodiment, the AAV serotype may be, or have, an AAV6 sequence comprising the K531E mutation (SEQ ID NO:5 of U.S. Pat. No. 9,546,112).

In some embodiments, the AAV serotype may be, or have, a mutation in the AAV1 sequence, as described in in United States Publication No. US 20130224836, the contents of which are herein incorporated by reference in their entirety, such as, but not limited to, at least one of the surface-exposed tyrosine residues, preferably, at positions 252, 273, 445, 701, 705 and 731 of AAV1 (SEQ ID NO: 2 of US 20130224836) substituted with another amino acid, preferably with a phenylalanine residue. In some embodiments, the AAV serotype may be, or have, a mutation in the AAV9 sequence, such as, but not limited to, at least one of the surface-exposed tyrosine residues, preferably, at positions 252, 272, 444, 500, 700, 704 and 730 of AAV2 (SEQ ID NO: 4 of US 20130224836) substituted with another amino acid, preferably with a phenylalanine residue. In some embodiments, the tyrosine residue at position 446 of AAV9 (SEQ ID NO: 6 US 20130224836) is substituted with a phenylalanine residue.

In some embodiments, the serotype may be AAV2 or a variant thereof, as described in International Publication No, WO2016130589, herein incorporated by reference in its entirety. The amino acid sequence of AAV2 may comprise N587A, E548A, or N708A mutations. In some embodiments, the amino acid sequence of any AAV may comprise a V708K mutation.

In some embodiments, the AAV serotype may be, or have, a sequence as described in United States Publication No, US20030138772, the contents of which are herein incorporated by reference in their entirety, such as, but not limited to, AAV1 (SEQ ID NO: 6 and 64 of US20030138772), AAV2 (SEQ ID NO: 7 and 70 of US20030138772), AAV3 (SEQ ID NO: 8 and 71 of US20030138772), AAV4 (SEQ ID NO: 63 of US20030138772), AAV5 (SEQ ID NO: 114 of US20030138772), AAV6 (SEQ ID NO: 65 of US20030138772), AAV7 (SEQ ID NO: 1-3 of US20030138772), AAV8 (SEQ ID NO: 4 and 95 of US20030138772), AAV9 (SEQ ID NO: 5 and 100 of US20030138772), AAV10 (SEQ ID NO: 117 of US20030138772), AAV11 (SEQ ID NO: 118 of US20030138772), AAV12 (SEQ ID NO: 119 of US20030138772), AAVrh10 (amino acids 1 to 738 of SEQ ID NO: 81 of US20030138772), AAV16.3 (US20030138772 SEQ ID NO: 10), AAV29.3/bb.1 (US20030138772 SEQ ID NO: 11), AAV29.4 (US20030138772 SEQ ID NO: 12), AAV29.5/bb.2 (US20030138772 SEQ ID NO: 13), AAV1.3 (US20030138772 SEQ ID NO: 14), AAV13.3 (US20030138772 SEQ ID NO: 15), AAV24.1 (US20030138772 SEQ ID NO: 16), AAV27.3 (US20030138772 SEQ ID NO: 17), AAV7.2 (US20030138772 SEQ ID NO: 18), AAVC1 (US20030138772 SEQ ID NO: 19), AAVC3 (US20030138772 SEQ ID NO: 20), AAVC5 (US20030138772 SEQ ID NO: 21), AAVF1 (US20030138772 SEQ ID NO: 22), AAVF3 (US200:30138772 SEQ ID NO: 23), AAVF5 (US20030138772 SEQ ID NO: 24), AAVH6 (US20030138772 SEQ ID NO: 25). AAVH2 (US20030138772 SEQ ID NO: 26), AAV42-8 (US20030138772 SEQ ID NO: 27), AAV42-15 (US20030138772 SEQ ID NO: 28), AAV42-5b (US20030138772 SEQ ID NO: 29). AAV42-1b (US20030138772 SEQ ID NO: 30), AAV42-13 (US20030138772 SEQ ID NO: 31), AAV42-3a (US20030138772 SEQ ID NO: 32), AAV42-4 (US20030138772 SEQ ID NO: 33). AAV42-5a (13520030138772 SEQ ID NO: 34), AAV42-10 (US20030138772 SEQ ID NO: 35), AAV42-3b (US20030138772 SEQ ID NO: 36), AAV42-11 (US20030138772 SEQ ID NO: 37), AAV42-6b (US20030138772 SEQ ID NO: 38), AAV43-1 (US20030138772 SEQ ID NO: 39), AAV43-5 (13520030138772 SEQ ID NO: 40), AAV43-12 (US20030138772 SEQ ID NO: 41), AAV43-20 (US20030138772 SEQ ID NO: 42), AAV43-21 (US20030138772 SEQ ID NO: 43), AAV43-23 (US20030138772 SEQ ID NO: 44), AAV43-25 (US20030138772 SEQ ID NO: 45), AAV44.1 (US20030138772 SEQ ID NO: 46), AAV44.5 (US20030138772 SEQ ID NO: 47), AAV223.1 (US20030138772 SEQ ID NO: 48), AAV223.2 (US20030138772 SEQ ID NO: 49), AAV223.4 (US20030138772 SEQ ID NO: 50), AAV223.5 (US20030138772 SEQ ID NO: 51), AAV223.6 (US20030138772 SEQ ID NO: 52), AAV223.7 (US20030138772 SEQ ID NO: 53), AAVA3.4 (US20030138772 SEQ ID NO: 54), AAVA3.5 (US20030138772 SEQ ID NO: 55), AAVA3.7 (US20030138772 SEQ ID NO: 56), AAVA3.3 (US20030138772 SEQ ID NO: 57), AAV42.12 (US20030138772 SEQ ID NO: 58), AAV44.2 (US20030138772 SEQ ID NO: 59), AAV42-2 (US20030138772 SEQ ID NO: 9), or variants thereof.

In some embodiments, the AAV serotype may be, or have, a sequence as described in United States Publication No. US20150159173, the contents of which are herein incorporated by reference in their entirety, such as, but not limited to, AAV2 (SEQ ID NO: 7 and 23 of US20150159173), rh20 (SEQ ID NO: 1 of US20150159173), rh32/33 (SEQ ID NO: 2 of US20150159173), rh39 (SEQ ID NO: 3, 20 and 36 of US20150159173), rh46 (SEQ ID NO: 4 and 22 of US20150159173), rh73 (SEQ ID NO: 5 of US20150159173), rh74 (SEQ ID NO: 6 of US20150159173), AAV6.1 (SEQ ID NO: 29 of US20150159173), rh.8 (SEQ ID NO: 41 of US20150159173), rh.48.1 (SEQ ID NO: 44 of US20150159173), hu.44 (SEQ ID NO: 45 of US20150159173), hu.29 (SEQ ID NO: 42 of US20150159173), hu.48 (SEQ ID NO: 38 of US20150159173), rh54 (SEQ ID NO: 49 of US20150159173), AAV2 (SEQ ID NO: 7 of US20150159173), cy.5 (SEQ ID NO: 8 and 24 of US20150159173), rh.10 (SEQ ID NO: 9 and 25 of US20150159173), rh.13 (SEQ ID NO: 10 and 26 of US20150159173), AAV1 (SEQ ID NO: 11 and 27 of US20150159173), AAV3 (SEQ ID NO: 12 and 28 of US20150159173), AAV6 (SEQ ID NO: 13 and 29 of US20150159173) AAV7 (SEQ ID NO: 14 and 30 of US20150159173), AAV8 (SEQ ID NO: 15 and 31 of US20150159173), hu.13 (SEQ ID NO: 16 and 32 of US20150159173), hu.26 (SEQ ID NO: 17 and 33 of US20150159173), hu.37 (SEQ ID NO: 18 and 34 of US20150159173), hu.53 (SEQ ID NO: 19 and 35 of US20150159173), rh.43 (SEQ ID NO: 21 and 37 of US20150159173), rh2 (SEQ ID NO: 39 of US20150159173), rh.37 (SEQ ID NO: 40 of US20150159173), rh.64 (SEQ ID NO: 43 of US20150159173), rh.48 (SEQ ID NO: 44 of US20150159173), ch.5 (SEQ ID NO 46 of US20150159173), rh.67 (SEQ ID NO: 47 of US201.50159173), rh.58 (SEQ ID NO: 48 of US20150159173), or variants thereof including, but not limited to Cy5R1, Cy5R2, Cy5R3, Cy5R4, rh.13R, rh.37R2, rh.2R, rh.8R, rh.48.1, rh.48.2, rh.48.1.2, hu.44R.1., hu.44R2, hu.44R3, hu.29R, ch.5R1, rh64R1, rh64R2, AAV6.2, AAV6.1, AAV6.12, hu.48R1, hu.48R2, and hu.48R3.

In some embodiments, the AAV serotype may be, or have, a sequence as described in U.S. Pat. No. 7,198,951, the contents of which are herein incorporated by reference in their entirety, such as, but not limited to, AAV9 (SEQ ID NO: 1-3 of U.S. Pat. No. 7,198,951), AAV2 (SEQ ID NO: 4 of U.S. Pat. No. 7,198,951), AAV1 (SEQ ID NO: 5 of U.S. Pat. No. 7,198,951), AAV3 (SEQ ID NO: 6 of U.S. Pat. No. 7,198,951), and AAV8 (SEQ ID NO: 7 of U.S. Pat. No. 7,198,951).

In some embodiments, the AAV serotype may be, or have, a mutation in the AAV9 sequence as described by N Pulicherla et al. (Molecular Therapy 19(6):1070-1078 (2011), herein incorporated by reference in its entirety), such as but not limited to, AAV9.9, AAV9.11, AAV9.13-A,AV9.16-NAV9,24, AAV9.45, AAV9.47, AAV9.61, AAV9.68, AAV9.84.

In some embodiments, the AAV serotype may be, or have, a sequence as described in U.S. Pat. No. 6,156,303, the contents of which are herein incorporated by reference in their entirety, such as, but not limited to, AAV 3B (SEQ ID NO: 1 and 10 of U.S. Pat. No. 6,156,303), AAV6 (SEQ ID NO: 2, 7 and 11 of U.S. Pat. No. 6,156,303), AAV2 (SEQ ID NO: 3 and 8 of U.S. Pat. No. 6,156,303), AAV3A (SEQ ID NO: 4 and 9, of U.S. Pat. No. 6,156,303), or derivatives thereof.

In some embodiments, the AAV serotype may be, or have, a sequence as described in United States Publication No. US20140359799, the contents of which are herein incorporated by reference in their entirety, such as, but not limited to, AAV8 (SEQ ID NO: 1 of US20140359799), AAVDJ (SEQ ID NO: 2 and 3 of US20140359799), or variants thereof.

In some embodiments, the serotype may be AAVDJ (or AAV-DJ) or a variant thereof, such as AAVDJ8 (or AAV-DJ8), as described by Grimm et al. (Journal of Virology 82(12): 5887-5911 (2008), herein incorporated by reference in its entirety). The amino acid sequence of AAVDJ8 may comprise two or more mutations in order to remove the heparin binding domain (HBD). As a non-limiting example, the AAV-DJ sequence described as SEQ ID NO: 1 in U.S. Pat. No. 7,588,772, the contents of which are herein incorporated by reference in their entirety, may comprise two mutations: (1) R587Q where arginine (R; Arg) at amino acid 587 is changed. to glutamine (Q; Gln) and (2) R590T where arginine (R; Arg) at amino acid 590 is changed to threonine (T; Thr). As another non-limiting example, may comprise three mutations: (1) K406R where lysine (K; Lys) at amino acid 406 is changed to arginine (R; Arg), (2) R587Q where arginine (R; Arg) at amino acid 587 is changed to glutamine (Q; Gln) and (3) 8590T where arginine (R; Arg) at amino acid 590 is changed to threonine (T; Thr).

In some embodiments, the AAV serotype may be, or have, a sequence of AAV4 as described in International Publication No. WO1998011244, the contents of which are herein incorporated by reference in their entirety, such as, hut not limited to AAV4 (SEQ ID NO: 1-20 of WO1998011244).

In some embodiments, the AAV serotype may be, or have, a mutation in the AAV2 sequence to generate AAV2G9 as described in International Publication No. WO2014144229 and herein incorporated by reference in its entirety.

In some embodiments, the AAV serotype may be, or have, a sequence as described in International Publication No. WO2005033321, the contents of which are herein incorporated by reference in their entirety, such as, but not limited to AAV3-3 (SEQ ID NO: 217 of WO2005033321), AAV1 (SEQ ID NO: 219 and 202 of WO2005033321), AAV106.1/hu.37 (SEQ ID No: 10 of WO2005033321), AAV114.3/hu.40 (SEQ ID No: 11 of WO2005033321), AAV1.27.2/hu.41 (SEQ ID NO:6 and 8 of WO2005033321), AAV128.3/hu.44 (SEQ ID No: 81 of WO2005033321), AAV130.4/hu.48 (SEQ ID NO: 78 of WO2005033321), AAV145.1/hu.53 (SEQ ID No: 176 and 177 of WO2005033321), AAV145.6/hu.56 (SEQ ID NO: 168 and 192 of WO2005033321), AAV16.12/hu.11 (SEQ ID NO: 153 and 57 of WO2005033321), AAV16.8/hu.10 (SEQ ID NO: 156 and 56 of WO2005033321), AAV161.10/hu.60 (SEQ ID No: 170 of WO2005033321), AAV161.6/hu.61 (SEQ ID No: 174 of WO2005033321), AAV1-7/rh.48 (SEQ ID NO: 32 of WO2005033321), AAV1-8/rh.49 (SEQ ID NOs: 103 and 25 of WO2005033321), AAV2 (SEQ ID NO: 211 and 221 of WO2005033321.), AAV2-15/rh.62 (SEQ ID No: 33 and 114 of WO2005033321), AAV2-3/rh.61 (SEQ ID NO: 21 of WO2005033321), AAV2-4/rh.50 (SEQ ID No: 23 and 108 of WO2005033321), AAV2-5/rh.51 (SEQ ID NO: 104 and 22 of WO2005033321), AAV3.1/hu.6 (SEQ ID NO: 5 and 84 of WO2005033321), AAV3.1/hu.9 (SEQ ID NO: 155 and 58 of WO2005033321), AAV3-11/rh.53 (SEQ ID NO: 186 and 176 of WO2005033321), AAV3-3 (SEQ ID NO: 200 of WO2005033321), AAV33.12/hu.17 (SEQ ID NO:4 of WO2005033321), AAV33.4/hu.15 (SEQ ID No: 50 of WO2005033321), AAV33.8/hu.16 (SEQ ID No: 51 of WO2005033321), AAV3-9/rh.52 (SEQ ID NO: 96 and 18 of WO2005033321), AAV4-19/rh.55 (SEQ ID NO: 117 of WO2005033321), AAV4-4 (SEQ ID NO: 201 and 218 of WO2005033321), AAV4-9/rh.54 (SEQ ID NO: 116 of WO2005033321), AAV5 (SEQ ID NO: 199 and 216 of WO2005033321), AAV52.1/hu.20 (SEQ ID NO: 63 of WO2005033321), AAV52/hu.19 (SEQ ID NO: 133 of WO2005033321), AAV5-22/rh.58 (SEQ ID No: 27 of WO2005033321), AAV5-3/rh.57 (SEQ ID NO: 105 of WO2005033321), AAV5-3/rh.57 (SEQ ID No: 26 of WO2005033321), AAV58.2./hu.25 (SEQ ID No: 49 of WO2005033321), AAV6 (SEQ ID NO: 203 and 220 of WO2005033321), AAV7 (SEQ ID NO: 222 and 213 of WO2005033321), AAV7.3/hu.7 (SEQ ID No: 55 of WO2005033321), AAV8 (SEQ ID NO: 223 and 214 of WO2005033321), AAVH-1/hu.1 (SEQ ID No: 46 of WO2005033321), AAVH-5/hu.3 (SEQ ID No: 44 of WO2005033321), AAVhu.1 (SEQ m NO: 144 of WO2005033321), AAVhu.10 (SEQ ID NO: 156 of WO2005033321), AAVhu.11 (SEQ ID NO: 153 of WO2005033321), AAVhu.12 (WO2005033321 SEQ ID NO: 59), AAVhu.13 (SEQ ID NO: 129 of WO2005033321), AAVhu-14/AAV9 (SEQ ID NO: 123 and 3 of WO2005033321), AAVhu.15 (SEQ ID NO: 147 of WO2005033321), AAVhu.16 (SEQ ID NO: 148 of WO2005033321), AAVhu.17 (SEQ ID NO: 83 of WO2005033321), AAVhu.18 (SEQ ID NO: 149 of WO2005033321), AAVhu.19 (SEQ ID NO: 133 of WO2005033321), AAVhu.2 (SEQ ID NO: 143 of WO2005033321), AAVhu.20 (SEQ ID NO: 134 of WO2005033321), AAVhu.21 (SEQ ID NO: 135 of WO2005033321), AAVhu.22 (SEQ ID NO: 138 of WO2005033321), AAVhu.23.2 (SEQ ID NO: 137 of WO2005033321), AAVhu.24 (SEQ ID NO: 136 of WO2005033321), AAVhu.25 (SEQ ID NO: 146 of WO2005033321), AAVhu.27 (SEQ ID NO: 140 of WO2005033321), AAVhu.29 (SEQ ID NO: 132 of WO2005033321), AAVhu.3 (SEQ ID NO: 145 of WO2005033321), AAVhu.31 (SEQ ID NO: 121 of WO2005033321), AAVhu.32 (SEQ ID NO: 122 of WO2005033321), AAVhu.34 (SEQ ID NO: 125 of WO2005033321), AAVhu.35 (SEQ ID NO: 164 of WO2005033321), AAVhu.37 (SEQ ID NO: 88 of WO2005033321), AAVhu.39 (SEQ ID NO: 102 of WO2005033321), AAVhu.4 (SEQ ID NO: 141 of WO2005033321), AAVhu.40 (SEQ ID NO: 87 of WO2005033321), AAVhu.41 (SEQ ID NO: 91 of WO2005033321), AAVhu.42 (SEQ ID NO: 85 of WO2005033321), AAVhu.43 (SEQ ID NO: 160 of WO2005033321), AAVhu.44 (SEQ ID NO: 144 of WO2005033321), AAVhu.45 (SEQ ID NO: 127 of WO2005033321), AAVhu.46 (SEQ ID NO: 159 of WO2005033321), AAVhu.47 (SEQ ID NO: 128 of WO2005033321), AAVhu.48 (SEQ ID NO: 157 of WO2005033321), AAVhu.49 (SEQ ID NO: 189 of WO2005033321), AAVhu.51 (SEQ ID NO: 190 of WO2005033321), AAVhu.52 (SEQ ID NO: 191 of WO2005033321), AAVhu.53 (SEQ ID NO: 186 of WO2005033321), AAVhu.54 (SEQ ID NO: 188 of WO2005033321), AAVhu.55 (SEQ ID NO: 187 of WO2005033321), AAVhu.56 (SEQ ID NO: 192 of WO2005033321), AAVhu.57 (SEQ ID NO: 193 of WO2005033321), AAVhu.58 (SEQ ID NO: 194 of WO2005033321), AAVhu.6 (SEQ ID NO: 84 of WO2005033321), AAVhu.60 (SEQ ID NO: 184 of WO2005033321), AAVhu.61. (SEQ ID NO: 185 of WO2005033321), AAVhu.63 (SEQ ID NO: 195 of WO2005033321), AAVhu.64 (SEQ ID NO: 196 of WO2005033321), AAVhu.66 (SEQ ID NO: 197 of WO2005033321), AAVhu.67 (SEQ ID NO: 198 of WO2005033321), AAVhu.7 (SEQ ID NO: 150 of WO20050:33321), AAVhu.8 (WO2005033321 SEQ ID NO: 12), AAVhu.9 (SEQ ID NO: 155 of WO2005033321), AAVLG-10/rh.40 (SEQ ID No: 14 of WO2005033321), AAVLG-4/rh.38 (SEQ ID NO: 86 of WO2005033321), AAVLG-4/rh.38 (SEQ ID No: 7 of WO2005033321), AAVN721-8/rh.43 (SEQ ID NO: 163 of WO2005033321), AAVN721-8/rh.43 (SEQ ID No: 43 of WO2005033321), AAVpi.1 (WO2005033321 SEQ ID NO: 28), AAVpi.2 (WO2005033321 SEQ ID NO: 30), AAVpi.3 (WO2005033321 SEQ ID NO: 29), AAVrh.38 (SEQ ID NO: 86 of WO2005033321), AAVrh.40 (SEQ ID NO: 92 of WO2005033321), AAVrh.43 (SEQ ID NO: 163 of WO2005033321), AAVrh.44 (WO2005033321 SEQ ID NO: 34), AAVrh.45 (WO2005033321 SEQ ID NO: 41), AAVrh.47 (WO2005033321 SEQ ID NO: 38), AAVrh.48 (SEQ ID NO: 115 of WO2005033321), AAVrh.49 (SEQ ID NO: 103 of WO2005033321), AAVrh.50 (SEQ ID NO: 108 of WO2005033321), AAVrh.51 (SEQ NO: 104 of WO2005033321), AAVrh.52 (SEQ ID NO: 96 of WO2005033321.), AAVrh.53 (SEQ ID NO: 97 of WO2005033321), AAVrh.55 (WO2005033321 SEQ ID NO: 37), AAVrh.56 (SEQ ID NO: 152 of WO2005033321), AAVrh.57 (SEQ ID NO: 105 of WO2005033321), AAVrh.58 (SEQ NO: 106 of WO2005033321), AAVrh.59 (WO2005033321 SEQ ID NO: 42), AAVrh.60 (WO2005033321 SEQ ID NO: 31). AAVrh.61 (SEQ ID NO: 107 of WO2005033321), AAVrh.62 (SEQ ID NO: 114 of WO2005033321), AAVrh.64 (SEQ ID NO: 99 of WO2005033321), AAVrh.65 (WO2005033321 SEQ ID NO: 35), AAVrh.68 (WO2005033321 SEQ ID NO: 16), AAVrh.69 (WO2005033321 SEQ ID NO: 39), AAVrh.70 (WO2005033321 SEQ ID NO: 20), AAVrh.72 (WO2005033321 SEQ ID NO: 9). or variants thereof including, but not limited to, AAVcy.2, AAVcy.3, AAVcy.4, AAVcy.5, AAVcy.6, AAVrh.12, AAVrh.17, AAVrh.18, AAVrh.19, AAVrh.21, AAVrh.22, AAVrh.23, AAVrh.24, AAVrh.25, AAVrh.25/42 15, AAVrh.31, AAVrh.32, AAVrh.33, AAVrh.34, AAVrh.35, AAVrh.36, AAVrh.37, AAVrh14. Non limiting examples of variants include SEQ ID NO: 13, 15, 17, 19, 24, 36, 40, 45, 47, 48, 51-54, 60-62, 64-77, 79. 80, 82, 89, 90, 93-95, 98, 100, 101, 109-113, 118-120, 124. 126, 131, 139, 142, 151, 154, 158, 161, 162, 165-183, 202, 204-212, 215, 219, 224-236, of WO2005033321, the contents of which are herein incorporated by reference in their entirety.

In some embodiments, the AAV serotype may be, or have, a sequence as described in International Publication No. WO2015168666, the contents of which are herein incorporated by reference in their entirety, such as, but not limited to, AAVrh8R (SEQ ID NO: 9 of WO2015168666), AAVrh8R A586R mutant (SEQ ID NO: 10 of WO2015168666), AAVrh8R, R533A mutant (SEQ ID NO: 11 of WO2015168666), or variants thereof.

In some embodiments, the AAV serotype may be, or have, a sequence as described in U.S. Pat. No. 9,233,131, the contents of which are herein incorporated by reference in their entirety, such as, but not limited to, AAVhE1.1 (SEQ ID NO:44 of U.S. Pat. No. 9,233,131), AAVhEr1.5 (SEQ ID NO:45 of U.S. Pat. No. 9,233,131), AAVhER1.14 (SEQ ID NO:46 of U.S. Pat. No. 9,233,131), AAVhEr1.8 (SEQ ID NO:47 of U.S. Pat. No. 9,233,131), AAVhEr1.16 (SEQ ID NO:48 of U.S. Pat. No. 9,233,131), AAVhEr1.18 (SEQ ID NO:49 of U.S. Pat. No. 9,233,131), AAVhEr1.35 (SEQ ID NO:50 of U.S. Pat. No. 9,233,131), AAVhEr1.7 (SEQ ID NO:51 of U.S. Pat. No. 9,233,131), AAVhEr1.36 (SEQ ID NO:52 of U.S. Pat. No. 9,233,131), AAVhEr2.29 (SEQ ID NO:53 of U.S. Pat. No. 9,233,131), AAVhEr2.4 (SEQ ID NO:54 of U.S. Pat. No. 9,233,131), AAVhEr2.16 (SEQ NO:55 of U.S. Pat. No. 9,233,131), AAVhEr2.30 (SEQ NO:56 of U.S. Pat. No. 9,233,131), AAVhEr2.31 (SEQ ID NO:58 of U.S. Pat. No. 9,233,131), AAVhEr2.36 (SEQ ID NO:57 of U.S. Pat. No. 9,233,131), AAVhER1.23 (SEQ ID NO:53 of U.S. Pat. No. 9,233,131), AAVhEr3.1 (SEQ ID NO:59 of U.S. Pat. No. 9,233,131), AAV2.5T (SEQ ID NO:42 of U.S. Pat. No. 9,23,3131), or variants thereof.

In some embodiments, the AAV serotype may be, or have, a sequence as described in United States Patent Publication No. US20150376607, the contents of which are herein incorporated by reference in their entirety, such as, but not limited to, AAV-PAEC (SEQ ID NO:1 of US20150376607), AAV-LK01 (SEQ ID NO:2 of US20150376607)-NAV-LK02 (SEQ ID NO:3 of US20150376607), AAV-LK03 (SEQ m NO:4 of US20150376607), AAV-LK04 (SEQ ID NO:5 of US20150376607), AAV-LK05 (SEQ ID NO:6 of US20150376607), AAV-LK06 (SEQ ID NO:7 of US20150376607), AAV-LK07 (SEQ ID NO:8 of US20150376607), AAV-LK08 (SEQ NO:9 of US20150376607), AAV-LK09 (SEQ NO:10 of US20150376607), AAV-LK10 (SEQ ID NO:11 of US20150376607), AAV-LK11 (SEQ NO:12 of US20150376607), AAV-LK12 (SEQ ID No:13 of US20150376607), AAV-LK13 (SEQ NO:14 of US20150376607), AAV-LK14 (SEQ NO:15 of US20150376607), AAV-LK15 (SEQ ID NO:16 of US20150376607)-AAV-LK16 (SEQ ID NO:17 of US20150376607), AAV-LK17 (SEQ ID NO:18 of US20150376607), AAV-LK18 (SEQ ID NO:19 of US20150376607), AAV-LK19 (SEQ ID NO:20 of US20150376607), AAV-PAEC.2 (SEQ ID NO:21 of US20150376607), AAV-PAEC4 (SEQ ID NO:22 of US20150376607), AAV-PAEC6 (SEQ ID NO:23 of US20150376607), AAV-PAEC7 (SEQ ID NO:24 of US20150376607), AAV-PAEC8 (SEQ ID NO:25 of US20150376607), AAV-PAEC 11 (SEQ ID NO:26 of US20150376607), AAV-PAEC12 (SEQ ID NO:27, of US20150376607), or variants thereof.

In some embodiments, the AAV serotype may be, or have, a sequence as described in U.S. Pat. No. 9,163,261, the contents of which are herein incorporated by reference in their entirety, such as, but not limited to, AAV-2-pre-miRNA-101 (SEQ ID NO: 1 U.S. Pat. No. 9,163,261), or variants thereof.

In some embodiments, the AAV serotype may be, or have, a sequence as described in United States Patent Publication No. US20150376240, the contents of which are herein incorporated by reference in their entirety, such as, but not limited to, AAV-811 (SEQ ID NO: 6 of US20150376240), AAV-8b (SEQ ID NO: 5 of US20150376240), AAV-h (SEQ ID NO: 2 of US20150376240), AAV-b (SEQ ID NO: 1 of US20150376240), or variants thereof.

In some embodiments, the AAV serotype may be, or have, a sequence as described in United States Patent Publication No. US20160017295, the contents of which are herein incorporated by reference in their entirety, such as, but not limited to, AAV SM 10-2 (SEQ ID NO: 22 of US20160017295), AAV Shuffle 100-1 (SEQ m NO: 23 of US20160017295), AAV Shuffle 100-3 (SEQ ID NO: 24 of US20160017295), AAV Shuffle 100-7 (SEQ ID NO: 25 of US20160017295), AAV Shuffle 10-2 (SEQ ID NO: 34 of US20160017295), AAV Shuffle 10-6 (SEQ ID NO: 35 of US20160017295), AAV Shuffle 10-8 (SEQ ID NO: 36 of US20160017295), AAV Shuffle 100-2 (SEQ m NO: 37 of US20160017295), AAV SM 10-1 (SEQ ID NO: 38 of US20160017295), AAV SM 10-8 (SEQ ID NO: 39 of US20160017295), AAV SM 100-3 (SEQ ID NO: 40 of US20160017295), AAV SM 100-10 (SEQ ID NO: 41 of US20160017295), or variants thereof.

In some embodiments, the AAV serotype may be, or have, a sequence as described in United States Patent Publication No. US20150238550, the contents of which are herein incorporated by reference in their entirety, such as, but not limited to, BNP61 AAV (SEQ ID NO: 1 of US20150238550), BNP62 AAV (SEQ ID NO: 3 of US20150238550), BNP63 AAV (SEQ ID NO: 4 of US20150238550), or variants thereof.

In some embodiments, the AAV serotype may be or may have a sequence as described in United States Patent Publication No. US20150315612, the contents of which are herein incorporated by reference in their entirety, such as, but not limited to, AAVrh.50 (SEQ ID NO: 108 of US20150315612), AAVrh.43 (SEQ ID NO: 163 of US20150315612), AAVrh.62 (SEQ ID NO: 114 of US20150315612), AAVrh.48 (SEQ ID NO: 115 of US20150315612), AAVhu.19 (SEQ ID NO: 133 of US20150315612), AAVhu.11 (SEQ ID NO: 153 of US20150315612), AAVhu.53 (SEQ ID NO: 186 of US20150315612), AAV4-8/rh.64 (SEQ ID No: 15 of US20150315612), AAVLG-9/hu.39 (SEQ ID No: 24 of US20150315612), AAV54.5/hu.23 (SEQ ID No: 60 of US20150315612), AAV54.2/hu.22 (SEQ ID No: 67 of US20150315612), AAV54.7/hu.24 (SEQ ID No: 66 of US20150315612), AAV54.1/hu.21 (SEQ ID No: 65 of US20150315612), AAV54.4R/hu.27 (SEQ ID No: 64 of US20150315612), AAV46.2/hu.28 (SEQ ID No: 68 of US20150315612), AAV46.6/hu.29 (SEQ ID No: 69 of US20150315612), AAV128.1/hu.43 (SEQ ID No: 80 of US20150315612), or variants thereof.

In some embodiments, the AAV serotype may be, or have, a sequence as described in International Publication No. WO2015121501, the contents of which are herein incorporated by reference in their entirety, such as, but not limited to, true type AAV (ttAAV) (SEQ ID NO: 2 of WO2015121501), “UPenn AAV10” (SEQ ID NO: 8 of WO2015121501), “Japanese AAV10” (SEQ ID NO: 9 of WO2015121501), or variants thereof.

According to the present disclosure, AAV capsid serotype selection or use may be from a variety of species. In some embodiments, the AAV may be an avian AAV (AAAV). The AAAV serotype may be, or have, a sequence as described in U.S. Pat. No. 9,238,800, the contents of which are herein incorporated by reference in their entirety, such as, but not limited to, AAAV (SEQ ID NO: 1, 2, 4, 6, 8, 10, 12, and 14 of U.S. Pat. No. 9,238,800), or variants thereof.

In some embodiments, the AAV may be a bovine AAV (BAAV). The BAAV serotype may be, or have, a sequence as described in U.S. Pat. No. 9,193,769, the contents of which are herein incorporated by reference in their entirety, such as, but not limited to, BAAV (SEQ ID NO: 1 and 6 of U.S. Pat. No. 9,193,769), or variants thereof. The BAAV serotype may be or have a sequence as described in U.S. Pat. No. 7,427,396, the contents of which are herein incorporated by reference in their entirety, such as, but not limited to, BAAV (SEQ ID NO: 5 and 6 of U.S. Pat. No. 7,427,396), or variants thereof.

In some embodiments, the AAV may be a caprine AAV. The caprine AAV serotype may be, or have, a sequence as described in U.S. Pat. No. 7,427,396, the contents of which are herein incorporated by reference in their entirety, such as, but not limited to, caprine AAV (SEQ ID NO: 3 of U.S. Pat. No. 7,427,396), or variants thereof.

In other embodiments the AAV may be engineered as a hybrid AAV from two or more parental serotypes. In some embodiments, the AAV may be AAV2G9 which comprises sequences from AAV2 and AAV9. The AAV2G9 AAV serotype may be, or have, a sequence as described in United States Patent Publication No. US20160017005, the contents of which are herein incorporated by reference in its entirety.

In some embodiments, the AAV may be a serotype generated by the AAV9 capsid library with mutations in amino acids 390-627 (VPI numbering) as described by Pulicherla et al. (Molecular Therapy 19(6):1070-1078 (2011), the contents of which are herein incorporated by reference in their entirety. The serotype and corresponding nucleotide and amino acid. substitutions may be, but is not limited to, AAV9.1 (G1594C; D532H), AAV6.2 (31418A and T1436X; V473D and I479K), AAV9.3 (11238A; F413Y), AAV9.4 (T1250C and A1617T; F417S), AAV9.5 (A1235G, A13141, A1642G, 017601; Q412R, 1548A, A587V), AAV9.6 (T1231A; F411I), AAV9.9 (G1203A, G1785T; W595C), AAV9.10 (A1500G, T1676C; M559T), AAV9.11 (A1425T, A1702C, A1769T; T568P, Q590L), AAV9.13 (A1369C, A1720T; N457H, T574S), AAV9.14 (T1340A, T1362C, T1560C, G1713A; L4471H), AAV9.16 (A1775T; Q592L), AAV9.24 (T1507C, T1521G; W503R), AAV9.26 (A1337G, A1769C; Y446C, Q590P), AAV9.33 (A1667C; D556A), AAV9.34 (A1534G, C1794T; N512D). AAV9.35 (A1289T, T1450A, C1494T, A1515T, C1794A, G1816A; Q430L, Y484N, N98K, V606I), AAV9.40 (A1694T; E565V), AAV9.41 (A1348T, T1362C; T450S), AAV9.44 (A1684C, A1701T, A1737G; N562H, K567N), AAV9.45 (A1492T, C1804T; N498Y, L602F), AAV9.46 (G1441C, T1525C, T1549G; G481R, W509R, L517V), 9.47 (G1241A, G1358A, A1669G, C1745T; S414N, G453D, K557E, T582I), AAV9.48 (C1445T, A1736T; P482L, Q579L), AAV9.50 (A1638T, C1683T, T1805A; Q546H, L602H), AAV9.53 (G1301A, A1405C, C1664T; G1811T; R134Q, S469R, A555V, G604V). AAV9.54 (C1531A, T1609A; L511I, L537M)-NAV9.55 (T1605A; F535L), AAV9.58 (C1475T, C1579A; T492I, H527N), AAV.59 (T1336C; Y446H), AAV9.61 (A1493T; N498I), AAV9.64 (C1531A, A1617T; L511I), AAV9.65 (C1335T, T1530C, C1568A; A523D), AAV9.68 (C1510A; P504T), AAV9.80 (G1441A,;G481R), AAV9.83 (C1402A, A1500T; P468T, E500D), AAV9.87 (T1464C, T1468C; S490P). AAV9.90 (A1196T; Y399F), AAV9.91 (T1316G, A1583T, C1782G, T1806C; L439R, K528I), AAV9.93 (A1273G, A1421G, A1638C, C1712T, G1732A, A1744T; A1832T; S425G, Q474R, Q546H, P571L, G578R, T582S, D611V), AAV9.94 (A1675T; M559L) and AAV9.95 (T1605A; F535L).

In some embodiments, the AAV serotype may be, or have, a sequence as described in International Publication No. WO2016049230, the contents of which are herein incorporated by reference in their entirety, such as, but not limited to AAVF1/HSC1 (SEQ ID NO: 2 and 20 of WO2016049230), AAVF2/HSC2 (SEQ ID NO: 3 and 21 of WO2016049230), AAVE3/HSC3 (SEQ ID NO: 5 and 22 of WO2016049230), AAVF4/HSC4 (SEQ ID NO: 6 and 23 of WO2016049230), AAVF5/HSC5 (SEQ ID NO: 11 and 25 of WO2016049230), AAVF6/HSC6 (SEQ ID NO: 7 and 24 of WO2016049230), AAVF7/HSC7 (SEQ ID NO: 8 and 27 of WO2016049230), AAVF8/HSC8 (SEQ ID NO: 9 and 28 of WO2016049230), AAVF9/HSC9 (SEQ ID NO: 10 and 29 of WO2016049230), AAVF11/HSC11 (SEQ ID NO: 4 and 26 of WO2016049230), AAVF12/HSC12 (SEQ ID NO: 12 and 30 of WO2016049230), AAVF13/HSC13 (SEQ ID NO: 14 and 31 of WO2016049230), AAVF14/HSC14 (SEQ ID NO: 15 and 32 of WO2016049230), AAVF15/HSC15 (SEQ ID NO: 16 and 33 of WO2016049230), AAVF16/HSC16 (SEQ ID NO: 17 and 34 of WO2016049230), AAVF17/HSC17 (SEQ ID NO: 13 and 35 of WO2016049230), or variants or derivatives thereof.

In some embodiments, the AAV serotype may be, or have, a sequence as described in U.S. Pat. No. 8,734,809, the contents of which are herein incorporated by reference in their entirety, such as, but not limited to, AAV CBr-E1 (SEQ ID NO: 13 and 87 of U.S. Pat. No. 8,734,809), AAV CBr-E2 (SEQ ID NO: 14 and 88 of U.S. Pat. No. 8,734,809), AAV CBr-E3 (SEQ ID NO: 15 and 89 of U.S. Pat. No. 8,734,809), AAV CBr-E4 (SEQ ID NO: 16 and 90 of U.S. Pat. No. 8,734,809), AAV CBr-E5 (SEQ ID NO: 17 and 91 of U.S. Pat. No. 8,734,809), AAV CBr-e5 (SEQ ID NO: 18 and 92 of U.S. Pat. No. 8,734,809), AAV CBr-E6 (SEQ ID NO: 19 and 93 of U.S. Pat. No. 8,734,809), AAV CBr-E7 (SEQ ID NO: 20 and 94 of U.S. Pat. No. 8,734,809), AAV CBr-E8 (SEQ ID NO: 21 and 95 of U.S. Pat. No. 8,734,809), AAV CLv-D1 (SEQ ID NO: 22 and 96 of U.S. Pat. No. 8,734,809), AAV CLv-D2 (SEQ NO: 23 and 97 of U.S. Pat. No. 8,734,809), AAV CLv-D3 (SEQ m NO: 24 and 98 of U.S. Pat. No. 8,734,809), AAV CLv-D4 (SEQ ID NO: 25 and 99 of U.S. Pat. No. 8,734,809), AAV CLv-D5 (SEQ ID NO: 26 and 100 of U.S. Pat. No. 8,734,809), AAV CLv-D6 (SEQ ID NO: 27 and 101 of U.S. Pat. No. 8,734,809), AAV CLv-D7 (SEQ ID NO: 28 and 102 of U.S. Pat. No. 8,734,809), AAV CLv-D8 (SEQ NO: 29 and 103 of U.S. Pat. No. 8,734,809), AAV CIN-E1 (SEQ NO: 13 and 87 of U.S. Pat. No. 8,734,809), AAV CLv-R1 (SEQ ID NO: 30 and 104 of U.S. Pat. No. 8,734,809), AAV CLv-R2 (SEQ ID NO: 31 and 105 of U.S. Pat. No. 8,734,809), AAV CLv-R3 (SEQ ID NO: 32 and 106 of U.S. Pat. No. 8,734,809), AAV CLv-R4 (SEQ ID NO: 33 and 107 of U.S. Pat. No. 8,734,809), AAV CLv-R5 (SEQ ID NO: 34 and 108 of U.S. Pat. No. 8,734,809), AAV CLv-R6 (SEQ m NO: 35 and 109 of U.S. Pat. No. 8,734,809), AAV CLv-R7 (SEQ ID NO: 36 and 110 of U.S. Pat. No. 8,734,809), AAV CLv-R8 (SEQ ID NO: 37 and 111 of U.S. Pat. No. 8,734,809), AAV CLv-R9 (SEQ ID NO: 38 and 112 of U.S. Pat. No. 8,734,809), AAV CLg-F1 (SEQ ID NO: 39 and 113 of U.S. Pat. No. 8,734,809), AAV CLg-F2 (SEQ ID NO: 40 and 114 of U.S. Pat. No. 8,734,809), AAV CLg-F3 (SEQ ID NO: 41 and 115 of U.S. Pat. No. 8,734,809), AAV CLg-F4 (SEQ m NO: 42 and 116 of U.S. Pat. No. 8,734,809), AAV CLg-F5 (SEQ ID NO: 43 and 117 of U.S. Pat. No. 8,734,809), AAV CLg-F6 (SEQ ID NO: 43 and 117 of U.S. Pat. No. 8,734,809), AAV Clg-F7 (SEQ ID NO: 44 and 118 of U.S. Pat. No. 8,734,809), AAV CLg-F8 (SEQ ID NO: 43 and 117 of U.S. Pat. No. 8,734,809), AAV CSp-1 (SEQ ID NO: 45 and 119 of U.S. Pat. No. 8,734,809), AAV CSp-10 (SEQ ID NO: 46 and 120 of U.S. Pat. No. 8,734,809), CSp-11 (SEQ ID NO: 47 and 121 of U.S. Pat. No. 8,734,809), AAV CSp-2 (SEQ ID NO: 48 and 122 of U.S. Pat. No. 8,734,809), AAV CSp-3 (SEQ ID NO: 49 and 123 of U.S. Pat. No. 8,734,809), AAV CSp-4 (SEQ ID NO: 50 and 124 of U.S. Pat. No. 8,734,809), AAV CSp-6 (SEQ ID NO: 51 and 125 of U.S. Pat. No. 8,734,809), AAV CSp-7 (SEQ ID NO: 52 and 126 of U.S. Pat. No. 8,734,809), AAV CSp-8 (SEQ ID NO: 53 and 127 of U.S. Pat. No. 8,734,809), AAV CSp-9 (SEQ ID NO: 54 and 128 of U.S. Pat. No. 8,734,809), AAV CHt-2 (SEQ ID NO: 55 and 129 of U.S. Pat. No. 8,734,809), AAV CHt-3 (SEQ ID NO: 56 and 130 of U.S. Pat. No. 8,734,809), AAV CKd-1 (SEQ m NO: 57 and 131 of U.S. Pat. No. 8,734,809), AAV CKd-10 (SEQ ID NO: 58 and 132 of U.S. Pat. No. 8,734,809), AAV CKd-2 (SEQ ID NO: 59 and 133 of U.S. Pat. No. 8,734,809), AAV CKd-3 (SEQ ID NO: 60 and 134 of U.S. Pat. No. 8,734,809), AAV CKd-4 (SEQ ID NO: 61 and 135 of U.S. Pat. No. 8,734,809), AAV CKd-6 (SEQ ID NO: 62 and 136 of U.S. Pat. No. 8,734,809), AAV CKd-7 (SEQ ID NO: 63 and 137 of U.S. Pat. No. 8,734,809), AAV CKd-8 (SEQ ID NO: 64 and 138 of U.S. Pat. No. 8,734,809), AAV CLv-1 (SEQ ID NO: 35 and 139 of U.S. Pat. No. 8,734,809), AAV CLv-12 (SEQ ID NO: 66 and 140 of U.S. Pat. No. 8,734,809), AAV CLv-13 (SEQ ID NO: 67 and 141 of U.S. Pat. No. 8,734,809), AAV CLv-2 (SEQ ID NO: 68 and 142 of U.S. Pat. No. 8,734,809), AAV CLbv-3 (SEQ ID NO: 69 and 143 of U.S. Pat. No. 8,734,809), AAV (SEQ ID NO: 70 and 144 of U.S. Pat. No. 8,734,809), AAV CLv-6 (SEQ ID NO: 71 and 145 of U.S. Pat. No. 8,734,809), AAV CLv-8 (SEQ ID NO: 72 and 146 of U.S. Pat. No. 8,734,809), AAV CKd-B1 (SEQ ID NO: 73 and 147 of U.S. Pat. No. 8,734,809), AAV CKd-B2 (SEQ ID NO: 74 and 148 of U.S. Pat. No. 8,734,809), AAV CKd-B3 (SEQ ID NO: 75 and 149 of U.S. Pat. No. 8,734,809), AAV CKd-B4 (SEQ ID NO: 76 and 150 of U.S. Pat. No. 8,734,809), AAV CKd-B5 (SEQ NO: 77 and 151 of U.S. Pat. No. 8,734,809), AAV CKd-B6 (SEQ ID NO: 78 and 152 of U.S. Pat. No. 8,734,809), AAV CKd-B7 (SEQ ID NO: 79 and 153 of U.S. Pat. No. 8,734,809), AAV CKd-B8 (SEQ ID NO: 80 and 154 of U.S. Pat. No. 8,734,809), AAV CKd-H1 (SEQ ID NO: 81 and 155 of U.S. Pat. No. 8,734,809), AAV CKd-H2 (SEQ ID NO: 82 and 156 of U.S. Pat. No. 8,734,809), AAV CKd-H3 (SEQ ID NO: 83 and 157 of U.S. Pat. No. 8,734,809), AAV CKd-H4 (SEQ ID NO: 84 and 158 of 1358,734,809), AAV CKd-H5 (SEQ ID NO: 85 and 159 of U.S. Pat. No. 8,734,809), AAV CKd-H6 (SEQ ID NO: 77 and 151 of U.S. Pat. No. 8,734,809), AAV CHt-1 (SEQ ID NO: 86 and 160 of U.S. Pat. No. 8,734,809)-NAV (SEQ ID NO: 171 of U.S. Pat. No. 8,734,809), AAV CLv1-2 (SEQ ID NO: 172 of U.S. Pat. No. 8,734,809), AAV CLv1-3 (SEQ ID NO: 173 of U.S. Pat. No. 8,734,809), AAV CLv1-4 (SEQ ID NO: 174 of U.S. Pat. No. 8,734,809), AAV Clv1-7 (SEQ ID NO: 175 of U.S. Pat. No. 8,734,809), AAV Clv1-8 (SEQ ID NO: 176 of U.S. Pat. No. 8,734,809), AAV Clv1-9 (SEQ ID NO: 177 of U.S. Pat. No. 8,734,809), AAV Clv1-10 (SEQ ID NO: 178 of U.S. Pat. No. 8,734,809), AAV.VR-355 (SEQ ID NO: 181 of U.S. Pat. No. 8,734,809). AAV.hu.48R3 (SEQ ID NO: 183 of U.S. Pat. No. 8,734,809), or variants or derivatives thereof.

In some embodiments, the AAV serotype may he, or have, a sequence as described in International Publication No. WO2016065001, the contents of which are herein incorporated by reference in their entirety, such as, but not limited to AAV CHt-P2 (SEQ ID NO: 1 and 51 of WO2016065001), AAV CHt-P5 (SEQ ID NO: 2 and 52 of WO2016065001), AAV CHt-P9 (SEQ ID NO: 3 and 53 of WO2016065001). AAV CBr-7.1 (SEQ ID NO: 4 and 54 of WO2016065001), AAV CBr-7.2 (SEQ ID NO: 5 and 55 of WO2016065001), AAV CBr-7.3 (SEQ ID NO: 6 and 56 of WO2016065001), AAV CBr-7.4 (SEQ ID NO: 7 and 57 of WO2016065001), AAV CBr-7.5 (SEQ ID NO: 8 and 58 of WO2016065001), AAV CBr-7.7 (SEQ ID NO: 9 and 59 of WO2016065001), AAV CBr-7.8 (SEQ ID NO: 10 and 60 of WO2016065001 AAV CBr-7.10 (SEQ ID NO: 11 and 61 of WO2016065001), AAV CKd-N3 (SEQ ID NO: 12 and 62 of WO2016065001), AAV CKd-N4 (SEQ m NO: 13 and 63 of WO2016065001), AAV CKd-N9 (SEQ ID NO: 14 and 64 of WO2016065001), AAV CLv-L4 (SEQ ID NO: 15 and 65 of WO2016065001), AAV (SEQ ID NO: 16 and 66 of WO2016065001), AAV CLv-L6 (SEQ ID NO: 17 and 67 of WO2016065001)-NAV CLv-K1 (SEQ ID NO: 18 and 68 of WO2016065001), AAV CLv-K3 (SEQ ID NO: 19 and 69 of WO2016065001), AAV CLv-K6 (SEQ ID NO: 20 and 70 of WO201.606500 AAV CLv-M1 (SEQ ID NO: 21 and 71 of WO2016065001), AAV CLv-M11 (SEQ ID NO: 22 and 72 of WO2016065001), AAV CLv-M2 (SEQ ID NO: 23 and 73 of WO2016065001), AAV CLv-M5 (SEQ ID NO: 24 and 74 of WO2016065001), AAV CLv-M6 (SEQ ID NO: 25 and 75 of WO2016065001), AAV CLv-M7 (SEQ ID NO: 26 and 76 of WO2016065001), AAV CL M8 (SEQ ID NO: 27 and 77 of WO2016065001), AAV CLv-M9 (SEQ ID NO: 28 and 78 of WO2016065001), AAV CHt-P1 (SEQ ID NO: 29 and 79 of WO2016065001), AAV CHt-P6 (SEQ ID NO: 30 and 80 of WO2016065001), AAV CHt-P8 (SEQ ID NO: 31 and 81 of WO2016065001), AAV CHt-6.1 (SEQ ID NO: 32 and 82 of WO2016065001), AAV CHt-6.10 (SEQ ID NO: 33 and 83 of WO2016065001), AAV CHt-6.5 (SEQ ID NO: 34 and 84 of WO2016065001), AAV CHt-6.6 (SEQ ID NO: 35 and 85 of WO2016065001), AAV CHt-6.7 (SEQ ID NO: 36 and 86 of WO2016065001), AAV CHt-6.8 (SEQ ID NO: 37 and 87 of WO2016065001), AAV CSp-8.10 (SEQ ID NO: 38 and 88 of WO2016065001), AAV CSp-8.2 (SEQ ID NO: 39 and 89 of WO2016065001), AAV CSp-8.4 (SEQ ID NO: 40 and 90 of WO2016065001), AAV CSp-8.5 (SEQ ID NO: 41 and 91 of WO2016065001), AAV CSp-8.6 (SEQ ID NO: 42 and 92 of WO2016065001), AAV CSp-8.7 (SEQ ID NO: 43 and 93 of WO2016065001), AAV CSp-8.8 (SEQ ID NO: 44 and 94 of WO2016065001), AAV CSp-8.9 (SEQ ID NO: 45 and 95 of WO2016065001), AAV CBr-B7.3 (SEQ ID NO: 46 and 96 of WO2016065001), AAV CBr-B7.4 (SEQ ID NO: 47 and 97 of WO2016065001), AAV3B (SEQ ID NO: 48 and 98 of WO2016065001), AAV4 (SEQ ID NO: 49 and 99 of WO2016065001), AAV5 (SEQ ID NO: 50 and 100 of WO2016065001), or variants or derivatives thereof.

In some embodiments, the AAV may be a serotype selected from any of those found in Table 1.

In some embodiments, the AAV may comprise a sequence, fragment or variant thereof, of the sequences in Table 1.

In some embodiments, the AAV may be encoded by a sequence, fragment or variant as described in Table 1.

TABLE 1
AAV Serotypes

| | SEQ | |
|---|---|---|
| Serotype | ID NO | Reference Information |
| AAV1 | 1 | US20150159173 SEQ ID NO: 11, |
| | | US20150315612 SEQ ID NO: 202 |
| AAV1 | 2 | US20160017295 SEQ ID NO: 1 |
| | | US20030138772 SEQ ID NO: 64, |
| | | US20150159173 SEQ ID NO: 27, |
| | | US20150315612 SEQ ID NO: 219, |
| | | U.S. Pat. No. 7,198,951 SEQ ID NO: 5 |
| AAV1 | 3 | US20030138772 SEQ ID NO: 6 |
| AAV1.3 | 4 | US20030138772 SEQ ID NO: 14 |
| AAV10 | 5 | US20030138772 SEQ ID NO: 117 |
| AAV10 | 6 | WO2015121501 SEQ ID NO: 9 |
| AAV10 | 7 | WO2015121501 SEQ ID NO: 8 |
| AAV11 | 8 | US20030138772 SEQ ID NO: 118 |
| AAV12 | 9 | US20030138772 SEQ ID NO: 119 |
| AAV2 | 10 | US20150159173 SEQ ID NO: 7, |
| | | US20150315612 SEQ ID NO: 211 |
| AAV2 | 11 | US20030138772 SEQ ID NO: 70, |
| | | US20150159173 SEQ ID NO: 23, |
| | | US20150315612 SEQ ID NO: 221, |
| | | US20160017295 SEQ ID NO: 2, |
| | | U.S. Pat. No. 6,156,303 SEQ ID NO: 4, |
| | | U.S. Pat. No. 7,198,951 SEQ ID NO: 4, |
| | | WO2015121501 SEQ ID NO: 1 |
| AAV2 | 12 | U.S. Pat. No. 6,156,303 SEQ ID NO: 8 |
| AAV2 | 13 | US20030138772 SEQ ID NO: 7 |
| AAV2 | 14 | U.S. Pat. No. 6,156,303 SEQ ID NO: 3 |
| AAV2.5T | 15 | U.S. Pat. No. 9,233,131 SEQ ID NO: 42 |
| AAV223.10 | 16 | US20030138772 SEQ ID NO: 75 |
| AAV223.2 | 17 | US20030138772 SEQ ID NO: 49 |
| AAV223.2 | 18 | US20030138772 SEQ ID NO: 76 |
| AAV223.4 | 19 | US20030138772 SEQ ID NO: 50 |
| AAV223.4 | 20 | US20030138772 SEQ ID NO: 73 |
| AAV223.5 | 21 | US20030138772 SEQ ID NO: 51 |
| AAV223.5 | 22 | US20030138772 SEQ ID NO: 74 |
| AAV223.6 | 23 | US20030138772 SEQ ID NO: 52 |
| AAV223.6 | 24 | US20030138772 SEQ ID NO: 78 |
| AAV223.7 | 25 | US20030138772 SEQ ID NO: 53 |
| AAV223.7 | 26 | US20030138772 SEQ ID NO: 77 |
| AAV29.3 | 27 | US20030138772 SEQ ID NO: 82 |
| AAV29.4 | 28 | US20030138772 SEQ ID NO: 12 |
| AAV29.5 | 29 | US20030138772 SEQ ID NO: 83 |
| AAV29.5 | 30 | US20030138772 SEQ ID NO: 13 |
| (AAVbb.2) | | |
| AAV3 | 31 | US20150159173 SEQ ID NO: 12 |
| AAV3 | 32 | US20030138772 SEQ ID NO: 71, |
| | | US20150159173 SEQ ID NO: 28, |
| | | US20160017295 SEQ ID NO: 3, |
| | | U.S. Pat. No. 7,198,951 SEQ ID NO: 6 |
| AAV3 | 33 | US20030138772 SEQ ID NO: 8 |
| AAV3.3b | 34 | US20030138772 SEQ ID NO: 72 |
| AAV3-3 | 35 | US20150315612 SEQ ID NO: 200 |
| AAV3-3 | 36 | US20150315612 SEQ ID NO: 217 |
| AAV3a | 37 | U.S. Pat. No. 6,156,303 SEQ ID NO: 5 |
| AAV3a | 38 | U.S. Pat. No. 6,156,303 SEQ ID NO: 9 |
| AAV3b | 39 | U.S. Pat. No. 6,156,303 SEQ ID NO: 6 |
| AAV3b | 40 | U.S. Pat. No. 6,156,303 SEQ ID NO: 10 |
| AAV3b | 41 | U.S. Pat. No. 6,156,303 SEQ ID NO: 1 |
| AAV4 | 42 | US20140348794 SEQ ID NO: 17 |
| AAV4 | 43 | US20140348794 SEQ ID NO: 5 |
| AAV4 | 44 | US20140348794 SEQ ID NO: 3 |
| AAV4 | 45 | US20140348794 SEQ ID NO: 14 |
| AAV4 | 46 | US20140348794 SEQ ID NO: 15 |
| AAV4 | 47 | US20140348794 SEQ ID NO: 19 |
| AAV4 | 48 | US20140348794 SEQ ID NO: 12 |
| AAV4 | 49 | US20140348794 SEQ ID NO: 13 |
| AAV4 | 50 | US20140348794 SEQ ID NO: 7 |
| AAV4 | 51 | US20140348794 SEQ ID NO: 8 |
| AAV4 | 52 | US20140348794 SEQ ID NO: 9 |
| AAV4 | 53 | US20140348794 SEQ ID NO: 2 |
| AAV4 | 54 | US20140348794 SEQ ID NO: 10 |
| AAV4 | 55 | US20140348794 SEQ ID NO: 11 |
| AAV4 | 56 | US20140348794 SEQ ID NO: 18 |
| AAV4 | 57 | US20030138772 SEQ ID NO: 63, |
| | | US20160017295 SEQ ID NO: 4, |
| | | US20140348794 SEQ ID NO: 4 |
| AAV4 | 58 | US20140348794 SEQ ID NO: 16 |
| AAV4 | 59 | US20140348794 SEQ ID NO: 20 |
| AAV4 | 60 | US20140348794 SEQ ID NO: 6 |
| AAV4 | 61 | US20140348794 SEQ ID NO: 1 |
| AAV42.2 | 62 | US20030138772 SEQ ID NO: 9 |
| AAV42.2 | 63 | US20030138772 SEQ ID NO: 102 |
| AAV42.3b | 64 | US20030138772 SEQ ID NO: 36 |
| AAV42.3B | 65 | US20030138772 SEQ ID NO: 107 |
| AAV42.4 | 66 | US20030138772 SEQ ID NO: 33 |
| AAV42.4 | 67 | US20030138772 SEQ ID NO: 88 |
| AAV42.8 | 68 | US20030138772 SEQ ID NO: 27 |
| AAV42.8 | 69 | US20030138772 SEQ ID NO: 85 |
| AAV43.1 | 70 | US20030138772 SEQ ID NO: 39 |
| AAV43.1 | 71 | US20030138772 SEQ ID NO: 92 |
| AAV43.12 | 72 | US20030138772 SEQ ID NO: 41 |
| AAV43.12 | 73 | US20030138772 SEQ ID NO: 93 |
| AAV43.20 | 74 | US20030138772 SEQ ID NO: 42 |
| AAV43.20 | 75 | US20030138772 SEQ ID NO: 99 |
| AAV43.21 | 76 | US20030138772 SEQ ID NO: 43 |
| AAV43.21 | 77 | US20030138772 SEQ ID NO: 96 |
| AAV43.23 | 78 | US20030138772 SEQ ID NO: 44 |
| AAV43.23 | 79 | US20030138772 SEQ ID NO: 98 |
| AAV43.25 | 80 | US20030138772 SEQ ID NO: 45 |
| AAV43.25 | 81 | US20030138772 SEQ ID NO: 97 |
| AAV43.5 | 82 | US20030138772 SEQ ID NO: 40 |
| AAV43.5 | 83 | US20030138772 SEQ ID NO: 94 |
| AAV4-4 | 84 | US20150315612 SEQ ID NO: 201 |
| AAV4-4 | 85 | US20150315612 SEQ ID NO: 218 |
| AAV44.1 | 86 | US20030138772 SEQ ID NO: 46 |
| AAV44.1 | 87 | US20030138772 SEQ ID NO: 79 |
| AAV44.5 | 88 | US20030138772 SEQ ID NO: 47 |
| AAV44.5 | 89 | US20030138772 SEQ ID NO: 80 |
| AAV4407 | 90 | US20150315612 SEQ ID NO: 90 |
| AAV5 | 91 | U.S. Pat. No. 7,427,396 SEQ ID NO: 1 |
| AAV5 | 92 | US20030138772 SEQ ID NO: 114 |
| AAV5 | 93 | US20160017295 SEQ ID NO: 5, |
| | | U.S. Pat. No. 7,427,396 SEQ ID NO: 2, |
| | | US20150315612 SEQ ID NO: 216 |
| AAV5 | 94 | US20150315612 SEQ ID NO: 199 |
| AAV6 | 95 | US20150159173 SEQ ID NO: 13 |
| AAV6 | 96 | US20030138772 SEQ ID NO: 65, |
| | | US20150159173 SEQ ID NO: 29, |
| | | US20160017295 SEQ ID NO: 6, |
| | | U.S. Pat. No. 6,156,303 SEQ ID NO: 7 |
| AAV6 | 97 | U.S. Pat. No. 6,156,303 SEQ ID NO: 11 |
| AAV6 | 98 | U.S. Pat. No. 6,156,303 SEQ ID NO: 2 |
| AAV6 | 99 | US20150315612 SEQ ID NO: 203 |
| AAV6 | 100 | US20150315612 SEQ ID NO: 220 |
| AAV6.1 | 101 | US20150159173 |
| AAV6.12 | 102 | US20150159173 |
| AAV6.2 | 103 | US20150159173 |
| AAV7 | 104 | US20150159173 SEQ ID NO: 14 |
| AAV7 | 105 | US20150315612 SEQ ID NO: 183 |
| AAV7 | 106 | US20030138772 SEQ ID NO: 2, |
| | | US20150159173 SEQ ID NO: 30, |
| | | US20150315612 SEQ ID NO: 181, |
| | | US20160017295 SEQ ID NO: 7 |
| AAV7 | 107 | US20030138772 SEQ ID NO: 3 |
| AAV7 | 108 | US20030138772 SEQ ID NO: 1, |
| | | US20150315612 SEQ ID NO: 180 |
| AAV7 | 109 | US20150315612 SEQ ID NO: 213 |
| AAV7 | 110 | US20150315612 SEQ ID NO: 222 |
| AAV8 | 111 | US20150159173 SEQ ID NO: 15 |
| AAV8 | 112 | US20150376240 SEQ ID NO: 7 |
| AAV8 | 113 | US20030138772 SEQ ID NO: 4, |
| | | US20150315612 SEQ ID NO: 182 |
| AAV8 | 114 | US20030138772 SEQ ID NO: 95, |
| | | US20140359799 SEQ ID NO: 1, |
| | | US20150159173 SEQ ID NO: 31, |
| | | US20160017295 SEQ ID NO: 8, |
| | | U.S. Pat. No. 7,198,951 SEQ ID NO: 7, |
| | | US20150315612 SEQ ID NO: 223 |
| AAV8 | 115 | US20150376240 SEQ ID NO: 8 |
| AAV8 | 116 | US20150315612 SEQ ID NO: 214 |
| AAV-8b | 117 | US20150376240 SEQ ID NO: 5 |
| AAV-8b | 118 | US20150376240 SEQ ID NO: 3 |
| AAV-8h | 119 | US20150376240 SEQ ID NO: 6 |
| AAV-8h | 120 | US20150376240 SEQ ID NO: 4 |
| AAV9 | 121 | US20030138772 SEQ ID NO: 5 |
| AAV9 | 122 | U.S. Pat. No. 7,198,951 SEQ ID NO: 1 |
| AAV9 | 123 | US20160017295 SEQ ID NO: 9 |
| AAV9 | 124 | US20030138772 SEQ ID NO: 100, |
| | | U.S. Pat. No. 7,198,951 SEQ ID NO: 2 |
| AAV9 | 125 | U.S. Pat. No. 7,198,951 SEQ ID NO: 3 |
| AAV9 | 126 | U.S. Pat. No. 7,906,111 SEQ ID NO: 3; |
| (AAVhu.14) | | WO2015038958 SEQ ID NO: 11 |
| AAV9 | 127 | U.S. Pat. No. 7,906,111 SEQ ID NO: 123; |
| (AAVhu.14) | | WO2015038958 SEQ ID NO: 2 |
| AAVA3.1 | 128 | US20030138772 SEQ ID NO: 120 |
| AAVA3.3 | 129 | US20030138772 SEQ ID NO: 57 |
| AAVA3.3 | 130 | US20030138772 SEQ ID NO: 66 |
| AAVA3.4 | 131 | US20030138772 SEQ ID NO: 54 |
| AAVA3.4 | 132 | US20030138772 SEQ ID NO: 68 |
| AAVA3.5 | 133 | US20030138772 SEQ ID NO: 55 |
| AAVA3.5 | 134 | US20030138772 SEQ ID NO: 69 |
| AAVA3.7 | 135 | US20030138772 SEQ ID NO: 56 |
| AAVA3.7 | 136 | US20030138772 SEQ ID NO: 67 |
| AAV29.3 | 137 | US20030138772 SEQ ID NO: 11 |
| (AAVbb.1) | | |
| AAVC2 | 138 | US20030138772 SEQ ID NO: 61 |
| AAV Ch.5 | 139 | US20150159173 SEQ ID NO: 46, |
| | | US20150315612 SEQ ID NO: 234 |
| AAVcy.2 | 140 | US20030138772 SEQ ID NO: 15 |
| (AAV13.3) | | |
| AAV24.1 | 141 | US20030138772 SEQ ID NO: 101 |
| AAVcy.3 | 142 | US20030138772 SEQ ID NO: 16 |
| (AAV24.1) | | |
| AAV27.3 | 143 | US20030138772 SEQ ID NO: 104 |
| AAVcy.4 | 144 | US20030138772 SEQ ID NO: 17 |
| (AAV27.3) | | |
| AAVcy.5 | 145 | US20150315612 SEQ ID NO: 227 |
| AAV7.2 | 146 | US20030138772 SEQ ID NO: 103 |
| AAVcy.5 | 147 | US20030138772 SEQ ID NO: 18 |
| (AAV7.2) | | |
| AAV16.3 | 148 | US20030138772 SEQ ID NO: 105 |
| AAVcy.6 | 149 | US20030138772 SEQ ID NO: 10 |
| (AAV16.3) | | |
| AAVcy.5 | 150 | US20150159173 SEQ ID NO: 8 |
| AAVcy.5 | 151 | US20150159173 SEQ ID NO: 24 |
| AAVCy.5R1 | 152 | US20150159173 |
| AAVCy.5R2 | 153 | US20150159173 |
| AAVCy.5R3 | 154 | US20150159173 |
| AAVCy.5R4 | 155 | US20150159173 |
| AAVDJ | 156 | US20140359799 SEQ ID NO: 3, |
| | | U.S. Pat. No. 7,588,772 SEQ ID NO: 2 |
| AAVDJ | 157 | US20140359799 SEQ ID NO: 2, |
| | | U.S. Pat. No. 7,588,772 SEQ ID NO: 1 |
| AAVDJ-8 | 158 | U.S. Pat. No. 7,588,772; Grimm et al |
| | | 2008 |
| AAVDJ-8 | 159 | U.S. Pat. No. 7,588,772; Grimm et al |
| | | 2008 |
| AAVF5 | 160 | US20030138772 SEQ ID NO: 110 |
| AAVH2 | 161 | US20030138772 SEQ ID NO: 26 |
| AAVH6 | 162 | US20030138772 SEQ ID NO: 25 |
| AAVhE1.1 | 163 | U.S. Pat. No. 9,233,131 SEQ ID NO: 44 |
| AAVhEr1.14 | 164 | U.S. Pat. No. 9,233,131 SEQ ID NO: 46 |
| AAVhEr1.16 | 165 | U.S. Pat. No. 9,233,131 SEQ ID NO: 48 |
| AAVhEr1.18 | 166 | U.S. Pat. No. 9,233,131 SEQ ID NO: 49 |
| AAVhEr1.23 | 167 | U.S. Pat. No. 9,233,131 SEQ ID NO: 53 |
| (AAVhEr2.29) | | |
| AAVhEr1.35 | 168 | U.S. Pat. No. 9,233,131 SEQ ID NO: 50 |
| AAVhEr1.36 | 169 | U.S. Pat. No. 9,233,131 SEQ ID NO: 52 |
| AAVhEr1.5 | 170 | U.S. Pat. No. 9,233,131 SEQ ID NO: 45 |
| AAVhEr1.7 | 171 | U.S. Pat. No. 9,233,131 SEQ ID NO: 51 |
| AAVhEr1.8 | 172 | U.S. Pat. No. 9,233,131 SEQ ID NO: 47 |
| AAVhEr2.16 | 173 | U.S. Pat. No. 9,233,131 SEQ ID NO: 55 |
| AAVhEr2.30 | 174 | U.S. Pat. No. 9,233,131 SEQ ID NO: 56 |
| AAVhEr2.31 | 175 | U.S. Pat. No. 9,233,131 SEQ ID NO: 58 |
| AAVhEr2.36 | 176 | U.S. Pat. No. 9,233,131 SEQ ID NO: 57 |
| AAVhEr2.4 | 177 | U.S. Pat. No. 9,233,131 SEQ ID NO: 54 |
| AAVhEr3.1 | 178 | U.S. Pat. No. 9,233,131 SEQ ID NO: 59 |
| AAVhu.1 | 179 | US20150315612 SEQ ID NO: 46 |
| AAVhu.1 | 180 | US20150315612 SEQ ID NO: 144 |
| AAVhu.10 | 181 | US20150315612 SEQ ID NO: 56 |
| (AAV16.8) | | |
| AAVhu.10 | 182 | US20150315612 SEQ ID NO: 156 |
| (AAV16.8) | | |
| AAVhu.11 | 183 | US20150315612 SEQ ID NO: 57 |
| (AAV16.12) | | |
| AAVhu.11 | 184 | US20150315612 SEQ ID NO: 153 |
| (AAV16.12) | | |
| AAVhu.12 | 185 | US20150315612 SEQ ID NO: 59 |
| AAVhu.12 | 186 | US20150315612 SEQ ID NO: 154 |
| AAVhu.13 | 187 | US20150159173 SEQ ID NO: 16, |
| | | US20150315612 SEQ ID NO: 71 |
| AAVhu.13 | 188 | US20150159173 SEQ ID NO: 32, |
| | | US20150315612 SEQ ID NO: 129 |
| AAVhu.136.1 | 189 | US20150315612 SEQ ID NO: 165 |
| AAVhu.140.1 | 190 | US20150315612 SEQ ID NO: 166 |
| AAVhu.140.2 | 191 | US20150315612 SEQ ID NO: 167 |
| AAVhu.145.6 | 192 | US20150315612 SEQ ID No: 178 |
| AAVhu.15 | 193 | US20150315612 SEQ ID NO: 147 |
| AAVhu.15 | 194 | US20150315612 SEQ ID NO: 50 |
| (AAV33.4) | | |
| AAVhu.156.1 | 195 | US20150315612 SEQ ID No: 179 |
| AAVhu.16 | 196 | US20150315612 SEQ ID NO: 148 |
| AAVhu.16 | 197 | US20150315612 SEQ ID NO: 51 |
| (AAV33.8) | | |
| AAVhu.17 | 198 | US20150315612 SEQ ID NO: 83 |
| AAVhu.17 | 199 | US20150315612 SEQ ID NO: 4 |
| (AAV33.12) | | |
| AAVhu.172.1 | 200 | US20150315612 SEQ ID NO: 171 |
| AAVhu.172.2 | 201 | US20150315612 SEQ ID NO: 172 |
| AAVhu.173.4 | 202 | US20150315612 SEQ ID NO: 173 |
| AAVhu.173.8 | 203 | US20150315612 SEQ ID NO: 175 |
| AAVhu.18 | 204 | US20150315612 SEQ ID NO: 52 |
| AAVhu.18 | 205 | US20150315612 SEQ ID NO: 149 |
| AAVhu.19 | 206 | US20150315612 SEQ ID NO: 62 |
| AAVhu.19 | 207 | US20150315612 SEQ ID NO: 133 |
| AAVhu.2 | 208 | US20150315612 SEQ ID NO: 48 |
| AAVhu.2 | 209 | US20150315612 SEQ ID NO: 143 |
| AAVhu.20 | 210 | US20150315612 SEQ ID NO: 63 |
| AAVhu.20 | 211 | US20150315612 SEQ ID NO: 134 |
| AAVhu.21 | 212 | US20150315612 SEQ ID NO: 65 |
| AAVhu.21 | 213 | US20150315612 SEQ ID NO: 135 |
| AAVhu.22 | 214 | US20150315612 SEQ ID NO: 67 |
| AAVhu.22 | 215 | US20150315612 SEQ ID NO: 138 |
| AAVhu.23 | 216 | US20150315612 SEQ ID NO: 60 |
| AAVhu.23.2 | 217 | US20150315612 SEQ ID NO: 137 |
| AAVhu.24 | 218 | US20150315612 SEQ ID NO: 66 |
| AAVhu.24 | 219 | US20150315612 SEQ ID NO: 136 |
| AAVhu.25 | 220 | US20150315612 SEQ ID NO: 49 |
| AAVhu.25 | 221 | US20150315612 SEQ ID NO: 146 |
| AAVhu.26 | 222 | US20150159173 SEQ ID NO: 17, |
| | | US20150315612 SEQ ID NO: 61 |
| AAVhu.26 | 223 | US20150159173 SEQ ID NO: 33, |
| | | US20150315612 SEQ ID NO: 139 |
| AAVhu.27 | 224 | US20150315612 SEQ ID NO: 64 |
| AAVhu.27 | 225 | US20150315612 SEQ ID NO: 140 |
| AAVhu.28 | 226 | US20150315612 SEQ ID NO: 68 |
| AAVhu.28 | 227 | US20150315612 SEQ ID NO: 130 |
| AAVhu.29 | 228 | US20150315612 SEQ ID NO: 69 |
| AAVhu.29 | 229 | US20150159173 SEQ ID NO: 42, |
| | | US20150315612 SEQ ID NO: 132 |
| AAVhu.29 | 230 | US20150315612 SEQ ID NO: 225 |
| AAVhu.29R | 231 | US20150159173 |
| AAVhu.3 | 232 | US20150315612 SEQ ID NO: 44 |
| AAVhu.3 | 233 | US20150315612 SEQ ID NO: 145 |
| AAVhu.30 | 234 | US20150315612 SEQ ID NO: 70 |
| AAVhu.30 | 235 | US20150315612 SEQ ID NO: 131 |
| AAVhu.31 | 236 | US20150315612 SEQ ID NO: 1 |
| AAVhu.31 | 237 | US20150315612 SEQ ID NO: 121 |
| AAVhu.32 | 238 | US20150315612 SEQ ID NO: 2 |
| AAVhu.32 | 239 | US20150315612 SEQ ID NO: 122 |
| AAVhu.33 | 240 | US20150315612 SEQ ID NO: 75 |
| AAVhu.33 | 241 | US20150315612 SEQ ID NO: 124 |
| AAVhu.34 | 242 | US20150315612 SEQ ID NO: 72 |
| AAVhu.34 | 243 | US20150315612 SEQ ID NO: 125 |
| AAVhu.35 | 244 | US20150315612 SEQ ID NO: 73 |
| AAVhu.35 | 245 | US20150315612 SEQ ID NO: 164 |
| AAVhu.36 | 246 | US20150315612 SEQ ID NO: 74 |
| AAVhu.36 | 247 | US20150315612 SEQ ID NO: 126 |
| AAVhu.37 | 248 | US20150159173 SEQ ID NO: 34, |
| | | US20150315612 SEQ ID NO: 88 |
| AAVhu.37 | 249 | US20150315612 SEQ ID NO: 10, |
| (AAV106.1) | | US20150159173 SEQ ID NO: 18 |
| AAVhu.38 | 250 | US20150315612 SEQ ID NO: 161 |
| AAVhu.39 | 251 | US20150315612 SEQ ID NO: 102 |
| AAVhu.39 | 252 | US20150315612 SEQ ID NO: 24 |
| (AAVLG-9) | | |
| AAVhu.4 | 253 | US20150315612 SEQ ID NO: 47 |
| AAVhu.4 | 254 | US20150315612 SEQ ID NO: 141 |
| AAVhu.40 | 255 | US20150315612 SEQ ID NO: 87 |
| AAVhu.40 | 256 | US20150315612 SEQ ID No: 11 |
| (AAV114.3) | | |
| AAVhu.41 | 257 | US20150315612 SEQ ID NO: 91 |
| AAVhu.41 | 258 | US20150315612 SEQ ID NO: 6 |
| (AAV127.2) | | |
| AAVhu.42 | 259 | US20150315612 SEQ ID NO: 85 |
| AAVhu.42 | 260 | US20150315612 SEQ ID NO: 8 |
| (AAV127.5) | | |
| AAVhu.43 | 261 | US20150315612 SEQ ID NO: 160 |
| AAVhu.43 | 262 | US20150315612 SEQ ID NO: 236 |
| AAVhu.43 | 263 | US20150315612 SEQ ID NO: 80 |
| (AAV128.1) | | |
| AAVhu.44 | 264 | US20150159173 SEQ ID NO: 45, |
| | | US20150315612 SEQ ID NO: 158 |
| AAVhu.44 | 265 | US20150315612 SEQ ID NO: 81 |
| (AAV128.3) | | |
| AAVhu.44R1 | 266 | US20150159173 |
| AAVhu.44R2 | 267 | US20150159173 |
| AAVhu.44R3 | 268 | US20150159173 |
| AAVhu.45 | 269 | US20150315612 SEQ ID NO: 76 |
| AAVhu.45 | 270 | US20150315612 SEQ ID NO: 127 |
| AAVhu.46 | 271 | US20150315612 SEQ ID NO: 82 |
| AAVhu.46 | 272 | US20150315612 SEQ ID NO: 159 |
| AAVhu.46 | 273 | US20150315612 SEQ ID NO: 224 |
| AAVhu.47 | 274 | US20150315612 SEQ ID NO: 77 |
| AAVhu.47 | 275 | US20150315612 SEQ ID NO: 128 |
| AAVhu.48 | 276 | US20150159173 SEQ ID NO: 38 |
| AAVhu.48 | 277 | US20150315612 SEQ ID NO: 157 |
| AAVhu.48 | 278 | US20150315612 SEQ ID NO: 78 |
| (AAV130.4) | | |
| AAVhu.48R1 | 279 | US20150159173 |
| AAVhu.48R2 | 280 | US20150159173 |
| AAVhu.48R3 | 281 | US20150159173 |
| AAVhu.49 | 282 | US20150315612 SEQ ID NO: 209 |
| AAVhu.49 | 283 | US20150315612 SEQ ID NO: 189 |
| AAVhu.5 | 284 | US20150315612 SEQ ID NO: 45 |
| AAVhu.5 | 285 | US20150315612 SEQ ID NO: 142 |
| AAVhu.51 | 286 | US20150315612 SEQ ID NO: 208 |
| AAVhu.51 | 287 | US20150315612 SEQ ID NO: 190 |
| AAVhu.52 | 288 | US20150315612 SEQ ID NO: 210 |
| AAVhu.52 | 289 | US20150315612 SEQ ID NO: 191 |
| AAVhu.53 | 290 | US20150159173 SEQ ID NO: 19 |
| AAVhu.53 | 291 | US20150159173 SEQ ID NO: 35 |
| AAVhu.53 | 292 | US20150315612 SEQ ID NO: 176 |
| (AAV145.1) | | |
| AAVhu.54 | 293 | US20150315612 SEQ ID NO: 188 |
| AAVhu.54 | 294 | US20150315612 SEQ ID No: 177 |
| (AAV145.5) | | |
| AAVhu.55 | 295 | US20150315612 SEQ ID NO: 187 |
| AAVhu.56 | 296 | US20150315612 SEQ ID NO: 205 |
| AAVhu.56 | 297 | US20150315612 SEQ ID NO: 168 |
| (AAV145.6) | | |
| AAVhu.56 | 298 | US20150315612 SEQ ID NO: 192 |
| (AAV145.6) | | |
| AAVhu.57 | 299 | US20150315612 SEQ ID NO: 206 |
| AAVhu.57 | 300 | US20150315612 SEQ ID NO: 169 |
| AAVhu.57 | 301 | US20150315612 SEQ ID NO: 193 |
| AAVhu.58 | 302 | US20150315612 SEQ ID NO: 207 |
| AAVhu.58 | 303 | US20150315612 SEQ ID NO: 194 |
| AAVhu.6 | 304 | US20150315612 SEQ ID NO: 5 |
| (AAV3.1) | | |
| AAVhu.6 | 305 | US20150315612 SEQ ID NO: 84 |
| (AAV3.1) | | |
| AAVhu.60 | 306 | US20150315612 SEQ ID NO: 184 |
| AAVhu.60 | 307 | US20150315612 SEQ ID NO: 170 |
| (AAV161.10) | | |
| AAVhu.61 | 308 | US20150315612 SEQ ID NO: 185 |
| AAVhu.61 | 309 | US20150315612 SEQ ID NO: 174 |
| (AAV161.6) | | |
| AAVhu.63 | 310 | US20150315612 SEQ ID NO: 204 |
| AAVhu.63 | 311 | US20150315612 SEQ ID NO: 195 |
| AAVhu.64 | 312 | US20150315612 SEQ ID NO: 212 |
| AAVhu.64 | 313 | US20150315612 SEQ ID NO: 196 |
| AAVhu.66 | 314 | US20150315612 SEQ ID NO: 197 |
| AAVhu.67 | 315 | US20150315612 SEQ ID NO: 215 |
| AAVhu.67 | 316 | US20150315612 SEQ ID NO: 198 |
| AAVhu.7 | 317 | US20150315612 SEQ ID NO: 226 |
| AAVhu.7 | 318 | US20150315612 SEQ ID NO: 150 |
| AAVhu.7 | 319 | US20150315612 SEQ ID NO: 55 |
| (AAV7.3) | | |
| AAVhu.71 | 320 | US20150315612 SEQ ID NO: 79 |
| AAVhu.8 | 321 | US20150315612 SEQ ID NO: 53 |
| AAVhu.8 | 322 | US20150315612 SEQ ID NO: 12 |
| AAVhu.8 | 323 | US20150315612 SEQ ID NO: 151 |
| AAVhu.9 | 324 | US20150315612 SEQ ID NO: 58 |
| (AAV3.1) | | |
| AAVhu.9 | 325 | US20150315612 SEQ ID NO: 155 |
| (AAV3.1) | | |
| AAV-LK01 | 326 | US20150376607 SEQ ID NO: 2 |
| AAV-LK01 | 327 | US20150376607 SEQ ID NO: 29 |
| AAV-LK02 | 328 | US20150376607 SEQ ID NO: 3 |
| AAV-LK02 | 329 | US20150376607 SEQ ID NO: 30 |
| AAV-LK03 | 330 | US20150376607 SEQ ID NO: 4 |
| AAV-LK03 | 331 | WO2015121501 SEQ ID NO: 12, |
| | | US20150376607 SEQ ID NO: 31 |
| AAV-LK04 | 332 | US20150376607 SEQ ID NO: 5 |
| AAV-LK04 | 333 | US20150376607 SEQ ID NO: 32 |
| AAV-LK05 | 334 | US20150376607 SEQ ID NO: 6 |
| AAV-LK05 | 335 | US20150376607 SEQ ID NO: 33 |
| AAV-LK06 | 336 | US20150376607 SEQ ID NO: 7 |
| AAV-LK06 | 337 | US20150376607 SEQ ID NO: 34 |
| AAV-LK07 | 338 | US20150376607 SEQ ID NO: 8 |
| AAV-LK07 | 339 | US20150376607 SEQ ID NO: 35 |
| AAV-LK08 | 340 | US20150376607 SEQ ID NO: 9 |
| AAV-LK08 | 341 | US20150376607 SEQ ID NO: 36 |
| AAV-LK09 | 342 | US20150376607 SEQ ID NO: 10 |
| AAV-LK09 | 343 | US20150376607 SEQ ID NO: 37 |
| AAV-LK10 | 344 | US20150376607 SEQ ID NO: 11 |
| AAV-LK10 | 345 | US20150376607 SEQ ID NO: 38 |
| AAV-LK11 | 346 | US20150376607 SEQ ID NO: 12 |
| AAV-LK11 | 347 | US20150376607 SEQ ID NO: 39 |
| AAV-LK12 | 348 | US20150376607 SEQ ID NO: 13 |
| AAV-LK12 | 349 | US20150376607 SEQ ID NO: 40 |
| AAV-LK13 | 350 | US20150376607 SEQ ID NO: 14 |
| AAV-LK13 | 351 | US20150376607 SEQ ID NO: 41 |
| AAV-LK14 | 352 | US20150376607 SEQ ID NO: 15 |
| AAV-LK14 | 353 | US20150376607 SEQ ID NO: 42 |
| AAV-LK15 | 354 | US20150376607 SEQ ID NO: 16 |
| AAV-LK15 | 355 | US20150376607 SEQ ID NO: 43 |
| AAV-LK16 | 356 | US20150376607 SEQ ID NO: 17 |
| AAV-LK16 | 357 | US20150376607 SEQ ID NO: 44 |
| AAV-LK17 | 358 | US20150376607 SEQ ID NO: 18 |
| AAV-LK17 | 359 | US20150376607 SEQ ID NO: 45 |
| AAV-LK18 | 360 | US20150376607 SEQ ID NO: 19 |
| AAV-LK18 | 361 | US20150376607 SEQ ID NO: 46 |
| AAV-LK19 | 362 | US20150376607 SEQ ID NO: 20 |
| AAV-LK19 | 363 | US20150376607 SEQ ID NO: 47 |
| AAV-PAEC | 364 | US20150376607 SEQ ID NO: 1 |
| AAV-PAEC | 365 | US20150376607 SEQ ID NO: 48 |
| AAV-PAEC11 | 366 | US20150376607 SEQ ID NO: 26 |
| AAV-PAEC11 | 367 | US20150376607 SEQ ID NO: 54 |
| AAV-PAEC12 | 368 | US20150376607 SEQ ID NO: 27 |
| AAV-PAEC12 | 369 | US20150376607 SEQ ID NO: 51 |
| AAV-PAEC13 | 370 | US20150376607 SEQ ID NO: 28 |
| AAV-PAEC13 | 371 | US20150376607 SEQ ID NO: 49 |
| AAV-PAEC2 | 372 | US20150376607 SEQ ID NO: 21 |
| AAV-PAEC2 | 373 | US20150376607 SEQ ID NO: 56 |
| AAV-PAEC4 | 374 | US20150376607 SEQ ID NO: 22 |
| AAV-PAEC4 | 375 | US20150376607 SEQ ID NO: 55 |
| AAV-PAEC6 | 376 | US20150376607 SEQ ID NO: 23 |
| AAV-PAEC6 | 377 | US20150376607 SEQ ID NO: 52 |
| AAV-PAEC7 | 378 | US20150376607 SEQ ID NO: 24 |
| AAV-PAEC7 | 379 | US20150376607 SEQ ID NO: 53 |
| AAV-PAEC8 | 380 | US20150376607 SEQ ID NO: 25 |
| AAV-PAEC8 | 381 | US20150376607 SEQ ID NO: 50 |
| AAVpi.1 | 382 | US20150315612 SEQ ID NO: 28 |
| AAVpi.1 | 383 | US20150315612 SEQ ID NO: 93 |
| AAVpi.2 | 384 | US20150315612 SEQ ID NO: 30 |
| AAVpi.2 | 385 | US20150315612 SEQ ID NO: 95 |
| AAVpi.3 | 386 | US20150315612 SEQ ID NO: 29 |
| AAVpi.3 | 387 | US20150315612 SEQ ID NO: 94 |
| AAVrh.10 | 388 | US20150159173 SEQ ID NO: 9 |
| AAVrh.10 | 389 | US20150159173 SEQ ID NO: 25 |
| AAV44.2 | 390 | US20030138772 SEQ ID NO: 59 |
| AAVrh.10 | 391 | US20030138772 SEQ ID NO: 81 |
| (AAV44.2) | | |
| AAV42.1B | 392 | US20030138772 SEQ ID NO: 90 |
| AAVrh.12 | 393 | US20030138772 SEQ ID NO: 30 |
| (AAV42.1b) | | |
| AAVrh.13 | 394 | US20150159173 SEQ ID NO: 10 |
| AAVrh.13 | 395 | US20150159173 SEQ ID NO: 26 |
| AAVrh.13 | 396 | US20150315612 SEQ ID NO: 228 |
| AAVrh3.13R | 397 | US20150159173 |
| AAV42.3A | 398 | US20030138772 SEQ ID NO: 87 |
| AAVrh.14 | 399 | US20030138772 SEQ ID NO: 32 |
| (AAV42.3a) | | |
| AAV42.5A | 400 | US20030138772 SEQ ID NO: 89 |
| AAVrh.17 | 401 | US20030138772 SEQ ID NO: 34 |
| (AAV42.5a) | | |
| AAV42.5B | 402 | US20030138772 SEQ ID NO: 91 |
| AAVrh.18 | 403 | US20030138772 SEQ ID NO: 29 |
| (AAV42.5b) | | |
| AAV42.6B | 404 | US20030138772 SEQ ID NO: 112 |
| AAVrh.19 | 405 | US20030138772 SEQ ID NO: 38 |
| (AAV42.6b) | | |
| AAVrh.2 | 406 | US20150159173 SEQ ID NO: 39 |
| AAVrh.2 | 407 | US20150315612 SEQ ID NO: 231 |
| AAVrh.20 | 408 | US20150159173 SEQ ID NO: 1 |
| AAV42.10 | 409 | US20030138772 SEQ ID NO: 106 |
| AAVrh.21 | 410 | US20030138772 SEQ ID NO: 35 |
| (AAV42.10) | | |
| AAV42.11 | 411 | US20030138772 SEQ ID NO: 108 |
| AAVrh.22 | 412 | US20030138772 SEQ ID NO: 37 |
| (AAV42.11) | | |
| AAV42.12 | 413 | US20030138772 SEQ ID NO: 113 |
| AAVrh.23 | 414 | US20030138772 SEQ ID NO: 58 |
| (AAV42.12) | | |
| AAV42.13 | 415 | US20030138772 SEQ ID NO: 86 |
| AAVrh.24 | 416 | US20030138772 SEQ ID NO: 31 |
| (AAV42.13) | | |
| AAV42.15 | 417 | US20030138772 SEQ ID NO: 84 |
| AAVrh.25 | 418 | US20030138772 SEQ ID NO: 28 |
| (AAV42.15) | | |
| AAVrh.2R | 419 | US20150159173 |
| AAVrh.31 | 420 | US20030138772 SEQ ID NO: 48 |
| (AAV223.1) | | |
| AAVC1 | 421 | US20030138772 SEQ ID NO: 60 |
| AAVrh.32 | 422 | US20030138772 SEQ ID NO: 19 |
| (AAVC1) | | |
| AAVrh.32/33 | 423 | US20150159173 SEQ ID NO: 2 |
| AAVrh.33 | 424 | US20030138772 SEQ ID NO: 20 |
| (AAVC3) | | |
| AAVC5 | 425 | US20030138772 SEQ ID NO: 62 |
| AAVrh.34 | 426 | US20030138772 SEQ ID NO: 21 |
| (AAVC5) | | |
| AAVF1 | 427 | US20030138772 SEQ ID NO: 109 |
| AAVrh.35 | 428 | US20030138772 SEQ ID NO: 22 |
| (AAVF1) | | |
| AAVF3 | 429 | US20030138772 SEQ ID NO: 111 |
| AAVrh.36 | 430 | US20030138772 SEQ ID NO: 23 |
| (AAVF3) | | |
| AAVrh.37 | 431 | US20030138772 SEQ ID NO: 24 |
| AAVrh.37 | 432 | US20150159173 SEQ ID NO: 40 |
| AAVrh.37 | 433 | US20150315612 SEQ ID NO: 229 |
| AAVrh.37R2 | 434 | US20150159173 |
| AAVrh.38 | 435 | US20150315612 SEQ ID NO: 7 |
| (AAVLG-4) | | |
| AAVrh.38 | 436 | US20450315612 SEQ ID NO: 86 |
| (AAVLG-4) | | |
| AAVrh.39 | 437 | US20150159173 SEQ ID NO: 20, |
| | | US20150315612 SEQ ID NO: 13 |
| AAVrh.39 | 438 | US20150159173 SEQ ID NO: 3, |
| | | US20150159173 SEQ ID NO: 36, |
| | | US20150315612 SEQ ID NO: 89 |
| AAVrh.40 | 439 | US20150315612 SEQ ID NO: 92 |
| AAVrh.40 | 440 | US20150315612 SEQ ID No: 14 |
| (AAVLG-10) | | |
| AAVrh.43 | 441 | US20150315612 SEQ ID NO: 43, |
| (AAVN721-8) | | US20150159173 SEQ ID NO: 21 |
| AAVrh.43 | 442 | US20450315612 SEQ ID NO: 163, |
| (AAVN721-8) | | US20150159173 SEQ ID NO: 37 |
| AAVrh.44 | 443 | US20150315612 SEQ ID NO: 34 |
| AAVrh.44 | 444 | US20150315612 SEQ ID NO: 111 |
| AAVrh.45 | 445 | US20150315612 SEQ ID NO: 41 |
| AAVrh.45 | 446 | US20150315612 SEQ ID NO: 109 |
| AAVrh.46 | 447 | US20150159173 SEQ ID NO: 22, |
| | | US20150315612 SEQ ID NO: 19 |
| AAVrh.46 | 448 | US20150159173 SEQ ID NO: 4, |
| | | US20150315612 SEQ ID NO: 101 |
| AAVrh.47 | 449 | US20150315612 SEQ ID NO: 38 |
| AAVrh.47 | 450 | US20150315612 SEQ ID NO: 118 |
| AAVrh.48 | 451 | US20150159173 SEQ ID NO: 44, |
| | | US20150315612 SEQ ID NO: 115 |
| AAVrh.48.1 | 452 | US20150159173 |
| AAVrh.48.1.2 | 453 | US20150159173 |
| AAVrh.48.2 | 454 | US20150159173 |
| AAVrh.48 | 455 | US20150315612 SEQ ID NO: 32 |
| (AAV1-7) | | |
| AAVrh.49 | 456 | US20150315612 SEQ ID NO: 25 |
| (AAV1-8) | | |
| AAVrh.49 | 457 | US20150315612 SEQ ID NO: 103 |
| (AAV1-8) | | |
| AAVrh.50 | 458 | US20150315612 SEQ ID NO: 23 |
| (AAV2-4) | | |
| AAVrh.50 | 459 | US20150315612 SEQ ID NO: 108 |
| (AAV2-4) | | |
| AAVrh.51 | 460 | US20150315612 SEQ ID No: 22 |
| (AAV2-5) | | |
| AAVrh.51 | 461 | US20150315612 SEQ ID NO: 104 |
| (AAV2-5) | | |
| AAVrh.52 | 462 | US20150315612 SEQ ID NO: 18 |
| (AAV3-9) | | |
| AAVrh.52 | 463 | US20150315612 SEQ ID NO: 96 |
| (AAV3-9) | | |
| AAVrh.53 | 464 | US20150315612 SEQ ID NO: 97 |
| AAVrh.53 | 465 | US20150315612 SEQ ID NO: 17 |
| (AAV3-11) | | |
| AAVrh.53 | 466 | US20150315612 SEQ ID NO: 186 |
| (AAV3-11) | | |
| AAVrh.54 | 467 | US20150315612 SEQ ID NO: 40 |
| AAVrh.54 | 468 | US20150159173 SEQ ID NO: 49, |
| | | US20150315612 SEQ ID NO: 116 |
| AAVrh.55 | 469 | US20150315612 SEQ ID NO: 37 |
| AAVrh.55 | 470 | US20150315612 SEQ ID NO: 117 |
| (AAV4-19) | | |
| AAVrh.56 | 471 | US20150315612 SEQ ID NO: 54 |
| AAVrh.56 | 472 | US20150315612 SEQ ID NO: 152 |
| AAVrh.57 | 473 | US20150315612 SEQ ID NO: 26 |
| AAVrh.57 | 474 | US20150315612 SEQ ID NO: 105 |
| AAVrh.58 | 475 | US20150315612 SEQ ID NO: 27 |
| AAVrh.58 | 476 | US20150159173 SEQ ID NO: 48, |
| | | US20150315612 SEQ ID NO: 106 |
| AAVrh.58 | 477 | US20150315612 SEQ ID NO: 232 |
| AAVrh.59 | 478 | US20150315612 SEQ ID NO: 42 |
| AAVrh.59 | 479 | US20150315612 SEQ ID NO: 110 |
| AAVrh.60 | 480 | US20150315612 SEQ ID NO: 31 |
| AAVrh.60 | 481 | US20150315612 SEQ ID NO: 120 |
| AAVrh.61 | 482 | US20150315612 SEQ ID NO: 107 |
| AAVrh.61 | 483 | US20150315612 SEQ ID NO: 21 |
| (AAV2-3) | | |
| AAVrh.62 | 484 | US20150315612 SEQ ID No: 33 |
| (AAV2-15) | | |
| AAVrh.62 | 485 | US20150315612 SEQ ID NO: 114 |
| (AAV2-15) | | |
| AAVrh.64 | 486 | US20150315612 SEQ ID No: 15 |
| AAVrh.64 | 487 | US20150159173 SEQ ID NO: 43, |
| | | US20150315612 SEQ ID NO: 99 |
| AAVrh.64 | 488 | US20150315612 SEQ ID NO: 233 |
| AAVRh.64R1 | 489 | US20150159173 |
| AAVRh.64R2 | 490 | US20150159173 |
| AAVrh.65 | 491 | US20150315612 SEQ ID NO: 35 |
| AAVrh.65 | 492 | US20150315612 SEQ ID NO: 112 |
| AAVrh.67 | 493 | US20150315612 SEQ ID NO: 36 |
| AAVrh.67 | 494 | US20150315612 SEQ ID NO: 230 |
| AAVrh.67 | 495 | US20150159173 SEQ ID NO: 47, |
| | | US20150315612 SEQ ID NO: 113 |
| AAVrh.68 | 496 | US20150315612 SEQ ID NO: 16 |
| AAVrh.68 | 497 | US20150315612 SEQ ID NO: 100 |
| AAVrh.69 | 498 | US20150315612 SEQ ID NO: 39 |
| AAVrh.69 | 499 | US20150315612 SEQ ID NO: 119 |
| AAVrh.70 | 500 | US20150315612 SEQ ID NO: 20 |
| AAVrh.70 | 501 | US20150315612 SEQ ID NO: 98 |
| AAVrh.71 | 502 | US20150315612 SEQ ID NO: 162 |
| AAVrh.72 | 503 | US20150315612 SEQ ID NO: 9 |
| AAVrh.73 | 504 | US20150159173 SEQ ID NO: 5 |
| AAVrh.74 | 505 | US20150159173 SEQ ID NO: 6 |
| AAVrh.8 | 506 | US20150159173 SEQ ID NO: 41 |
| AAVrh.8 | 507 | US20150315612 SEQ ID NO: 235 |
| AAVrh.8R | 508 | US20150159173, WO2015168666 |
| | | SEQ ID NO: 9 |
| AAVrh.8R | 509 | WO2015168666 SEQ ID NO: 10 |
| A586R | | |
| mutant | | |
| AAVrh.8R | 510 | WO2015168666 SEQ ID NO: 11 |
| R533A | | |
| mutant | | |
| BAAV | 511 | U.S. Pat. No. 9,193,769 SEQ ID NO: 8 |
| (bovine | | |
| AAV) | | |
| BAAV | 512 | U.S. Pat. No. 9,193,769 SEQ ID NO: 10 |
| (bovine | | |
| AAV) | | |
| BAAV | 513 | U.S. Pat. No. 9,193,769 SEQ ID NO: 4 |
| (bovine | | |
| AAV) | | |
| BAAV | 514 | U.S. Pat. No. 9,193,769 SEQ ID NO: 2 |
| (bovine | | |
| AAV) | | |
| BAAV | 515 | U.S. Pat. No. 9,193,769 SEQ ID NO: 6 |
| (bovine | | |
| AAV) | | |
| BAAV | 516 | U.S. Pat. No. 9,193,769 SEQ ID NO: 1 |
| (bovine | | |
| AAV) | | |
| BAAV | 517 | U.S. Pat. No. 9,193,769 SEQ ID NO: 5 |
| (bovine | | |
| AAV) | | |
| BAAV | 518 | U.S. Pat. No. 9,193,769 SEQ ID NO: 3 |
| (bovine | | |
| AAV) | | |
| BAAV | 519 | U.S. Pat. No. 9,193,769 SEQ ID NO: 11 |
| (bovine | | |
| AAV) | | |
| BAAV | 520 | U.S. Pat. No. 7,427,396 SEQ ID NO: 5 |
| (bovine | | |
| AAV) | | |
| BAAV | 521 | U.S. Pat. No. 7,427,396 SEQ ID NO: 6 |
| (bovine | | |
| AAV) | | |
| BAAV | 522 | U.S. Pat. No. 9,193,769 SEQ ID NO: 7 |
| (bovine | | |
| AAV) | | |
| BAAV | 523 | U.S. Pat. No. 9,193,769 SEQ ID NO: 9 |
| (bovine | | |
| AAV) | | |
| BNP61 AAV | 524 | US20150238550 SEQ ID NO: 1 |
| BNP61 AAV | 525 | US20150238550 SEQ ID NO: 2 |
| BNP62 AAV | 526 | US20150238550 SEQ ID NO: 3 |
| BNP63 AAV | 527 | US20150238550 SEQ ID NO: 4 |
| caprine AAV | 528 | U.S. Pat. No. 7,427,396 SEQ ID NO: 3 |
| caprine AAV | 529 | U.S. Pat. No. 7,427,396 SEQ ID NO: 4 |
| true type | 530 | WO2015121501 SEQ ID NO: 2 |
| AAV (ttAAV) | | |
| AAAV | 531 | U.S. Pat. No. 9,238,800 SEQ ID NO: 12 |
| (Avian AAV) | | |
| AAAV | 532 | U.S. Pat. No. 9,238,800 SEQ ID NO: 2 |
| (Avian AAV) | | |
| AAAV | 533 | U.S. Pat. No. 9,238,800 SEQ ID NO: 6 |
| (Avian AAV) | | |
| AAAV | 534 | U.S. Pat. No. 9,238,800 SEQ ID NO: 4 |
| (Avian AAV) | | |
| AAAV | 535 | U.S. Pat. No. 9,238,800 SEQ ID NO: 8 |
| (Avian AAV) | | |
| AAAV | 536 | U.S. Pat. No. 9,238,800 SEQ ID NO: 14 |
| (Avian AAV) | | |
| AAAV | 537 | U.S. Pat. No. 9,238,800 SEQ ID NO: 10 |
| (Avian AAV) | | |
| AAAV | 538 | U.S. Pat. No. 9,238,800 SEQ ID NO: 15 |
| (Avian AAV) | | |
| AAAV | 539 | U.S. Pat. No. 9,238,800 SEQ ID NO: 5 |
| (Avian AAV) | | |
| AAAV | 540 | U.S. Pat. No. 9,238,800 SEQ ID NO: 9 |
| (Avian AAV) | | |
| AAAV | 541 | U.S. Pat. No. 9,238,800 SEQ ID NO: 3 |
| (Avian AAV) | | |
| AAAV | 542 | U.S. Pat. No. 9,238,800 SEQ ID NO: 7 |
| (Avian AAV) | | |
| AAAV | 543 | U.S. Pat. No. 9,238,800 SEQ ID NO: 11 |
| (Avian AAV) | | |
| AAAV | 544 | U.S. Pat. No. 9,238,800 SEQ ID NO: 13 |
| (Avian AAV) | | |
| AAAV | 545 | U.S. Pat. No. 9,238,800 SEQ ID NO: 1 |
| (Avian AAV) | | |
| AAV Shuffle | 546 | US20160017295 SEQ ID NO: 23 |
| 100-1 | | |
| AAV Shuffle | 547 | US20160017295 SEQ ID NO: 11 |
| 100-1 | | |
| AAV Shuffle | 548 | US20160017295 SEQ ID NO: 37 |
| 100-2 | | |
| AAV Shuffle | 549 | US20160017295 SEQ ID NO: 29 |
| 100-2 | | |
| AAV Shuffle | 550 | US20160017295 SEQ ID NO: 24 |
| 100-3 | | |
| AAV Shuffle | 551 | US20160017295 SEQ ID NO: 12 |
| 100-3 | | |
| AAV Shuffle | 552 | US20160017295 SEQ ID NO: 25 |
| 100-7 | | |
| AAV Shuffle | 553 | US20160017295 SEQ ID NO: 13 |
| 100-7 | | |
| AAV Shuffle | 554 | US20160017295 SEQ ID NO: 34 |
| 10-2 | | |
| AAV Shuffle | 555 | US20160017295 SEQ ID NO: 26 |
| 10-2 | | |
| AAV Shuffle | 556 | US20160017295 SEQ ID NO: 35 |
| 10-6 | | |
| AAV Shuffle | 557 | US20160017295 SEQ ID NO: 27 |
| 10-6 | | |
| AAV Shuffle | 558 | US20160017295 SEQ ID NO: 36 |
| 10-8 | | |
| AAV Shuffle | 559 | US20160017295 SEQ ID NO: 28 |
| 10-8 | | |
| AAV SM 100-10 | 560 | US20160017295 SEQ ID NO: 41 |
| AAV SM 100-10 | 561 | US20160017295 SEQ ID NO: 33 |
| AAV SM 100-3 | 562 | US20160017295 SEQ ID NO: 40 |
| AAV SM 100-3 | 563 | US20160017295 SEQ ID NO: 32 |
| AAV SM 10-1 | 564 | US20160017295 SEQ ID NO: 38 |
| AAV SM 10-1 | 565 | US20160017295 SEQ ID NO: 30 |
| AAV SM 10-2 | 566 | US20160017295 SEQ ID NO: 10 |
| AAV SM 10-2 | 567 | US20160017295 SEQ ID NO: 22 |
| AAV SM 10-8 | 568 | US20160017295 SEQ ID NO: 39 |
| AAV SM 10-8 | 569 | US20160017295 SEQ ID NO: 31 |
| AAV SM 100-10 | 560 | US20160017295 SEQ ID NO: 41 |
| AAV SM 100-10 | 561 | US20160017295 SEQ ID NO: 33 |
| AAV SM 100-3 | 562 | US20160017295 SEQ ID NO: 40 |
| AAV SM 100-3 | 563 | US20160017295 SEQ ID NO: 32 |
| AAV SM 10-1 | 564 | US20160017295 SEQ ID NO: 38 |
| AAV SM 10-1 | 565 | US20160017295 SEQ ID NO: 30 |
| AAV SM 10-2 | 566 | US20160017295 SEQ ID NO: 10 |
| AAV SM 10-2 | 567 | US20160017295 SEQ ID NO: 22 |
| AAV SM 10-8 | 568 | US20160017295 SEQ ID NO: 39 |
| AAV SM 10-8 | 569 | US20160017295 SEQ ID NO: 31 |
| AAVF1/HSC1 | 570 | WO2016049230 SEQ ID NO: 20 |
| AAVF2/HSC2 | 571 | WO2016049230 SEQ ID NO: 21 |
| AAVF3/HSC3 | 572 | WO2016049230 SEQ ID NO: 22 |
| AAVF4/HSC4 | 573 | WO2016049230 SEQ ID NO: 23 |
| AAVF5/HSC5 | 574 | WO2016049230 SEQ ID NO: 25 |
| AAVF6/HSC6 | 575 | WO2016049230 SEQ ID NO: 24 |
| AAVF7/HSC7 | 576 | WO2016049230 SEQ ID NO: 27 |
| AAVF8/HSC8 | 577 | WO2016049230 SEQ ID NO: 28 |
| AAVF9/HSC9 | 578 | WO2016049230 SEQ ID NO: 29 |
| AAVF11/HSC11 | 579 | WO2016049230 SEQ ID NO: 26 |
| AAVF12/HSC12 | 580 | WO2016049230 SEQ ID NO: 30 |
| AAVF13/HSC13 | 581 | WO2016049230 SEQ ID NO: 31 |
| AAVF14/HSC14 | 582 | WO2016049230 SEQ ID NO: 32 |
| AAVF15/HSC15 | 583 | WO2016049230 SEQ ID NO: 33 |
| AAVF16/HSC16 | 584 | WO2016049230 SEQ ID NO: 34 |
| AAVF17/HSC17 | 585 | WO2016049230 SEQ ID NO: 35 |
| AAVF1/HSC1 | 586 | WO2016049230 SEQ ID NO: 2 |
| AAVF2/HSC2 | 587 | WO2016049230 SEQ ID NO: 3 |
| AAVF3/HSC3 | 588 | WO2016049230 SEQ ID NO: 5 |
| AAVF4/HSC4 | 589 | WO2016049230 SEQ ID NO: 6 |
| AAVF5/HSC5 | 590 | WO2016049230 SEQ ID NO: 11 |
| AAVF6/HSC6 | 591 | WO2016049230 SEQ ID NO: 7 |
| AAVF7/HSC7 | 592 | WO2016049230 SEQ ID NO: 8 |
| AAVF8/HSC8 | 593 | WO2016049230 SEQ ID NO: 9 |
| AAVF9/HSC9 | 594 | WO2016049230 SEQ ID NO: 10 |
| AAVF11/HSC11 | 595 | WO2016049230 SEQ ID NO: 4 |
| AAVF12/HSC12 | 596 | WO2016049230 SEQ ID NO: 12 |
| AAVF13/HSC13 | 597 | WO2016049230 SEQ ID NO: 14 |
| AAVF14/HSC14 | 598 | WO2016049230 SEQ ID NO: 15 |
| AAVF15/HSC15 | 599 | WO2016049230 SEQ ID NO: 16 |
| AAVF16/HSC16 | 600 | WO2016049230 SEQ ID NO: 17 |
| AAVF17/HSC17 | 601 | WO2016149230 SEQ ID NO: 13 |
| AAV CBr-E1 | 602 | U.S. Pat. No. 8,734,809 SEQ ID NO: 13 |
| AAV CBr-E2 | 603 | U.S. Pat. No. 8,734,809 SEQ ID NO: 14 |
| AAV CBr-E3 | 604 | U.S. Pat. No. 8,734,809 SEQ ID NO: 15 |
| AAV CBr-E4 | 605 | U.S. Pat. No. 8,734,809 SEQ ID NO: 16 |
| AAV CBr-E5 | 606 | U.S. Pat. No. 8,734,809 SEQ ID NO: 17 |
| AAV CBr-e5 | 607 | U.S. Pat. No. 8,734,809 SEQ ID NO: 18 |
| AAV CBr-E6 | 608 | U.S. Pat. No. 8,734,809 SEQ ID NO: 19 |
| AAV CBr-E7 | 609 | U.S. Pat. No. 8,734,809 SEQ ID NO: 20 |
| AAV CBr-E8 | 610 | U.S. Pat. No. 8,734,809 SEQ ID NO: 21 |
| AAV CLv-D1 | 611 | U.S. Pat. No. 8,734,809 SEQ ID NO: 22 |
| AAV CLv-D2 | 612 | U.S. Pat. No. 8,734,809 SEQ ID NO: 23 |
| AAV CLv-D3 | 613 | U.S. Pat. No. 8,734,809 SEQ ID NO: 24 |
| AAV CLv-D4 | 614 | U.S. Pat. No. 8,734,809 SEQ ID NO: 25 |
| AAV CLv-D5 | 615 | U.S. Pat. No. 8,734,809 SEQ ID NO: 26 |
| AAV CLv-D6 | 616 | U.S. Pat. No. 8,734,809 SEQ ID NO: 27 |
| AAV CLv-D7 | 617 | U.S. Pat. No. 8,734,809 SEQ ID NO: 28 |
| AAV CLv-D8 | 618 | U.S. Pat. No. 8,734,809 SEQ ID NO: 29 |
| AAV CLv-E1 | 619 | U.S. Pat. No. 8,734,809 SEQ ID NO: 13 |
| AAV CLv-R1 | 620 | U.S. Pat. No. 8,734,809 SEQ ID NO: 30 |
| AAV CLv-R2 | 621 | U.S. Pat. No. 8,734,809 SEQ ID NO: 31 |
| AAV CLv-R3 | 622 | U.S. Pat. No. 8,734,809 SEQ ID NO: 32 |
| AAV CLv-R4 | 623 | U.S. Pat. No. 8,734,809 SEQ ID NO: 33 |
| AAV CLv-R5 | 624 | U.S. Pat. No. 8,734,809 SEQ ID NO: 34 |
| AAV CLv-R6 | 625 | U.S. Pat. No. 8,734,809 SEQ ID NO: 35 |
| AAV CLv-R7 | 626 | U.S. Pat. No. 8,734,809 SEQ ID NO: 36 |
| AAV CLv-R8 | 627 | U.S. Pat. No. 8,734,809 SEQ ID NO: 37 |
| AAV CLv-R9 | 628 | U.S. Pat. No. 8,734,809 SEQ ID NO: 38 |
| AAV CLg-F1 | 629 | U.S. Pat. No. 8,734,809 SEQ ID NO: 39 |
| AAV CLg-F2 | 630 | U.S. Pat. No. 8,734,809 SEQ ID NO: 40 |
| AAV CLg-F3 | 631 | U.S. Pat. No. 8,734,809 SEQ ID NO: 41 |
| AAV CLg-F4 | 632 | U.S. Pat. No. 8,734,809 SEQ ID NO: 42 |
| AAV CLg-F5 | 633 | U.S. Pat. No. 8,734,809 SEQ ID NO: 43 |
| AAV CLg-F6 | 634 | U.S. Pat. No. 8,734,809 SEQ ID NO: 43 |
| AAV CLg-F7 | 635 | U.S. Pat. No. 8,734,809 SEQ ID NO: 44 |
| AAV CLg-F8 | 636 | U.S. Pat. No. 8,734,809 SEQ ID NO: 43 |
| AAV CSp-1 | 637 | U.S. Pat. No. 8,734,809 SEQ ID NO: 45 |
| AAV CSp-10 | 638 | U.S. Pat. No. 8,734,809 SEQ ID NO: 46 |
| AAV CSp-11 | 639 | U.S. Pat. No. 8,734,809 SEQ ID NO: 47 |
| AAV CSp-2 | 640 | U.S. Pat. No. 8,734,809 SEQ ID NO: 48 |
| AAV CSp-3 | 641 | U.S. Pat. No. 8,734,809 SEQ ID NO: 49 |
| AAV CSp-4 | 642 | U.S. Pat. No. 8,734,809 SEQ ID NO: 50 |
| AAV CSp-6 | 643 | U.S. Pat. No. 8,734,809 SEQ ID NO: 51 |
| AAV CSp-7 | 644 | U.S. Pat. No. 8,734,809 SEQ ID NO: 52 |
| AAV CSp-8 | 645 | U.S. Pat. No. 8,734,809 SEQ ID NO: 53 |
| AAV CSp-9 | 646 | U.S. Pat. No. 8,734,809 SEQ ID NO: 54 |
| AAV CHt-2 | 647 | U.S. Pat. No. 8,734,809 SEQ ID NO: 55 |
| AAV CHt-3 | 648 | U.S. Pat. No. 8,734,809 SEQ ID NO: 56 |
| AAV CKd-1 | 649 | U.S. Pat. No. 8,734,809 SEQ ID NO: 57 |
| AAV CKd-10 | 650 | U.S. Pat. No. 8,734,809 SEQ ID NO: 58 |
| AAV CKd-2 | 651 | U.S. Pat. No. 8,734,809 SEQ ID NO: 59 |
| AAV CKd-3 | 652 | U.S. Pat. No. 8,734,809 SEQ ID NO: 60 |
| AAV CKd-4 | 653 | U.S. Pat. No. 8,734,809 SEQ ID NO: 61 |
| AAV CKd-6 | 654 | U.S. Pat. No. 8,734,809 SEQ ID NO: 62 |
| AAV CKd-7 | 655 | U.S. Pat. No. 8,734,809 SEQ ID NO: 63 |
| AAV CKd-8 | 656 | U.S. Pat. No. 8,734,809 SEQ ID NO: 64 |
| AAV CLv-1 | 657 | U.S. Pat. No. 8,734,809 SEQ ID NO: 65 |
| AAV CLv-12 | 658 | U.S. Pat. No. 8,734,809 SEQ ID NO: 66 |
| AAV CLv-13 | 659 | U.S. Pat. No. 8,734,809 SEQ ID NO: 67 |
| AAV CLv-2 | 660 | U.S. Pat. No. 8,734,809 SEQ ID NO: 68 |
| AAV CLv-3 | 661 | U.S. Pat. No. 8,734,809 SEQ ID NO: 69 |
| AAV CLv-4 | 662 | U.S. Pat. No. 8,734,809 SEQ ID NO: 70 |
| AAV CLv-6 | 663 | U.S. Pat. No. 8,734,809 SEQ ID NO: 71 |
| AAV CLv-8 | 664 | U.S. Pat. No. 8,734,809 SEQ ID NO: 72 |
| AAV CKd-B1 | 665 | U.S. Pat. No. 8,734,809 SEQ ID NO: 73 |
| AAV CKd-B2 | 666 | U.S. Pat. No. 8,734,809 SEQ ID NO: 74 |
| AAV CKd-B3 | 667 | U.S. Pat. No. 8,734,809 SEQ ID NO: 75 |
| AAV CKd-B4 | 668 | U.S. Pat. No. 8,734,809 SEQ ID NO: 76 |
| AAV CKd-B5 | 669 | U.S. Pat. No. 8,734,809 SEQ ID NO: 77 |
| AAV CKd-B6 | 670 | U.S. Pat. No. 8,734,809 SEQ ID NO: 78 |
| AAV CKd-B7 | 671 | U.S. Pat. No. 8,734,809 SEQ ID NO: 79 |
| AAV CKd-B8 | 672 | U.S. Pat. No. 8,734,809 SEQ ID NO: 80 |
| AAV CKd-H1 | 673 | U.S. Pat. No. 8,734,809 SEQ ID NO: 81 |
| AAV CKd-H2 | 674 | U.S. Pat. No. 8,734,809 SEQ ID NO: 82 |
| AAV CKd-H3 | 675 | U.S. Pat. No. 8,734,809 SEQ ID NO: 83 |
| AAV CKd-H4 | 676 | U.S. Pat. No. 8,734,809 SEQ ID NO: 84 |
| AAV CKd-H5 | 677 | U.S. Pat. No. 8,734,809 SEQ ID NO: 85 |
| AAV CKd-H6 | 678 | U.S. Pat. No. 8,734,809 SEQ ID NO: 77 |
| AAV CHt-1 | 679 | U.S. Pat. No. 8,734,809 SEQ ID NO: 86 |
| AAV CLv1-1 | 680 | U.S. Pat. No. 8,734,809 SEQ ID NO: 171 |
| AAV CLv1-2 | 681 | U.S. Pat. No. 8,734,809 SEQ ID NO: 172 |
| AAV CLv1-3 | 682 | U.S. Pat. No. 8,734,809 SEQ ID NO: 173 |
| AAV CLv1-4 | 683 | U.S. Pat. No. 8,734,809 SEQ ID NO: 174 |
| AAV CLv1-7 | 684 | U.S. Pat. No. 8,734,809 SEQ ID NO: 175 |
| AAV CLv1-8 | 685 | U.S. Pat. No. 8,734,809 SEQ ID NO: 176 |
| AAV CLv1-9 | 686 | U.S. Pat. No. 8,734,809 SEQ ID NO: 177 |
| AAV CLv1-10 | 687 | U.S. Pat. No. 8,734,809 SEQ ID NO: 178 |
| AAV.VR-355 | 688 | U.S. Pat. No. 8,734,809 SEQ ID NO: 181 |
| AAV.hu.48R3 | 689 | U.S. Pat. No. 8,734,809 SEQ ID NO: 183 |
| AAV CBr-E1 | 690 | U.S. Pat. No. 8,734,809 SEQ ID NO: 87 |
| AAV CBr-E2 | 691 | U.S. Pat. No. 8,734,809 SEQ ID NO: 88 |
| AAV CBr-E3 | 692 | U.S. Pat. No. 8,734,809 SEQ ID NO: 89 |
| AAV CBr-E4 | 693 | U.S. Pat. No. 8,734,809 SEQ ID NO: 90 |
| AAV CBr-E5 | 694 | U.S. Pat. No. 8,734,809 SEQ ID NO: 91 |
| AAV CBr-e5 | 695 | U.S. Pat. No. 8,734,809 SEQ ID NO: 92 |
| AAV CBr-E6 | 696 | U.S. Pat. No. 8,734,809 SEQ ID NO: 93 |
| AAV CBr-E7 | 697 | U.S. Pat. No. 8,734,809 SEQ ID NO: 94 |
| AAV CBr-E8 | 698 | U.S. Pat. No. 8,734,809 SEQ ID NO: 95 |
| AAV CLv-D1 | 699 | U.S. Pat. No. 8,734,809 SEQ ID NO: 96 |
| AAV CLv-D2 | 700 | U.S. Pat. No. 8,734,809 SEQ ID NO: 97 |
| AAV CLv-D3 | 701 | U.S. Pat. No. 8,734,809 SEQ ID NO: 98 |
| AAV CLv-D4 | 702 | U.S. Pat. No. 8,734,809 SEQ ID NO: 99 |
| AAV CLv-D5 | 703 | U.S. Pat. No. 8,734,809 SEQ ID NO: 100 |
| AAV CLv-D6 | 704 | U.S. Pat. No. 8,734,809 SEQ ID NO: 101 |
| AAV CLv-D7 | 705 | U.S. Pat. No. 8,734,809 SEQ ID NO: 102 |
| AAV CLv-D8 | 706 | U.S. Pat. No. 8,734,809 SEQ ID NO: 103 |
| AAV CLv-E1 | 707 | U.S. Pat. No. 8,734,809 SEQ ID NO: 87 |
| AAV CLv-R1 | 708 | U.S. Pat. No. 8,734,809 SEQ ID NO: 104 |
| AAV CLv-R2 | 709 | U.S. Pat. No. 8,734,809 SEQ ID NO: 105 |
| AAV CLv-R3 | 710 | U.S. Pat. No. 8,734,809 SEQ ID NO: 106 |
| AAV CLv-R4 | 711 | U.S. Pat. No. 8,734,809 SEQ ID NO: 107 |
| AAV CLv-R5 | 712 | U.S. Pat. No. 8,734,809 SEQ ID NO: 108 |
| AAV CLv-R6 | 713 | U.S. Pat. No. 8,734,809 SEQ ID NO: 109 |
| AAV CLv-R7 | 714 | U.S. Pat. No. 8,734,809 SEQ ID NO: 110 |
| AAV CLv-R8 | 715 | U.S. Pat. No. 8,734,809 SEQ ID NO: 111 |
| AAV CLv-R9 | 716 | U.S. Pat. No. 8,734,809 SEQ ID NO: 112 |
| AAV CLg-F1 | 717 | U.S. Pat. No. 8,734,809 SEQ ID NO: 113 |
| AAV CLg-F2 | 718 | U.S. Pat. No. 8,734,809 SEQ ID NO: 114 |
| AAV CLg-F3 | 719 | U.S. Pat. No. 8,734,809 SEQ ID NO: 115 |
| AAV CLg-F4 | 720 | U.S. Pat. No. 8,734,809 SEQ ID NO: 116 |
| AAV CLg-F5 | 721 | U.S. Pat. No. 8,734,809 SEQ ID NO: 117 |
| AAV CLg-F6 | 722 | U.S. Pat. No. 8,734,809 SEQ ID NO: 117 |
| AAV CLg-F7 | 723 | U.S. Pat. No. 8,734,809 SEQ ID NO: 118 |
| AAV CLg-F8 | 724 | U.S. Pat. No. 8,734,809 SEQ ID NO: 117 |
| AAV CSp-1 | 725 | U.S. Pat. No. 8,734,809 SEQ ID NO: 119 |
| AAV CSp-10 | 726 | U.S. Pat. No. 8,734,809 SEQ ID NO: 120 |
| AAV CSp-11 | 727 | U.S. Pat. No. 8,734,809 SEQ ID NO: 121 |
| AAV CSp-2 | 728 | U.S. Pat. No. 8,734,809 SEQ ID NO: 122 |
| AAV CSp-3 | 729 | U.S. Pat. No. 8,734,809 SEQ ID NO: 123 |
| AAV CSp-4 | 730 | U.S. Pat. No. 8,734,809 SEQ ID NO: 124 |
| AAV CSp-6 | 731 | U.S. Pat. No. 8,734,809 SEQ ID NO: 125 |
| AAV CSp-7 | 732 | U.S. Pat. No. 8,734,809 SEQ ID NO: 126 |
| AAV CSp-8 | 733 | U.S. Pat. No. 8,734,809 SEQ ID NO: 127 |
| AAV CSp-9 | 734 | U.S. Pat. No. 8,734,809 SEQ ID NO: 128 |
| AAV CHt-2 | 735 | U.S. Pat. No. 8,734,809 SEQ ID NO: 129 |
| AAV CHt-3 | 736 | U.S. Pat. No. 8,734,809 SEQ ID NO: 130 |
| AAV CKd-1 | 737 | U.S. Pat. No. 8,734,809 SEQ ID NO: 131 |
| AAV CKd-10 | 738 | U.S. Pat. No. 8,734,809 SEQ ID NO: 132 |
| AAV CKd-2 | 739 | U.S. Pat. No. 8,734,809 SEQ ID NO: 133 |
| AAV CKd-3 | 740 | U.S. Pat. No. 8,734,809 SEQ ID NO: 134 |
| AAV CKd-4 | 741 | U.S. Pat. No. 8,734,809 SEQ ID NO: 135 |
| AAV CKd-6 | 742 | U.S. Pat. No. 8,734,809 SEQ ID NO: 136 |
| AAV CKd-7 | 743 | U.S. Pat. No. 8,734,809 SEQ ID NO: 137 |
| AAV CKd-8 | 744 | U.S. Pat. No. 8,734,809 SEQ ID NO: 138 |
| AAV CLv-1 | 745 | U.S. Pat. No. 8,734,809 SEQ ID NO: 139 |
| AAV CLv-12 | 746 | U.S. Pat. No. 8,734,809 SEQ ID NO: 140 |
| AAV CLv-13 | 747 | U.S. Pat. No. 8,734,809 SEQ ID NO: 141 |
| AAV CLv-2 | 748 | U.S. Pat. No. 8,734,809 SEQ ID NO: 142 |
| AAV CLv-3 | 749 | U.S. Pat. No. 8,734,809 SEQ ID NO: 143 |
| AAV CLv-4 | 750 | U.S. Pat. No. 8,734,809 SEQ ID NO: 144 |
| AAV CLv-6 | 751 | U.S. Pat. No. 8,734,809 SEQ ID NO: 145 |
| AAV CLv-8 | 752 | U.S. Pat. No. 8,734,809 SEQ ID NO: 146 |
| AAV CKd-B1 | 753 | U.S. Pat. No. 8,734,809 SEQ ID NO: 147 |
| AAV CKd-B2 | 754 | U.S. Pat. No. 8,734,809 SEQ ID NO: 148 |
| AAV CKd-B3 | 755 | U.S. Pat. No. 8,734,809 SEQ ID NO: 149 |
| AAV CKd-B4 | 756 | U.S. Pat. No. 8,734,809 SEQ ID NO: 150 |
| AAV CKd-B5 | 757 | U.S. Pat. No. 8,734,809 SEQ ID NO: 151 |
| AAV CKd-B6 | 758 | U.S. Pat. No. 8,734,809 SEQ ID NO: 152 |
| AAV CKd-B7 | 759 | U.S. Pat. No. 8,734,809 SEQ ID NO: 153 |
| AAV CKd-B8 | 760 | U.S. Pat. No. 8,734,809 SEQ ID NO: 154 |
| AAV CKd-H1 | 761 | U.S. Pat. No. 8,734,809 SEQ ID NO: 155 |
| AAV CKd-H2 | 762 | U.S. Pat. No. 8,734,809 SEQ ID NO: 156 |
| AAV CKd-H3 | 763 | U.S. Pat. No. 8,734,809 SEQ ID NO: 157 |
| AAV CKd-H4 | 764 | U.S. Pat. No. 8,734,809 SEQ ID NO: 158 |
| AAV CKd-H5 | 765 | U.S. Pat. No. 8,734,809 SEQ ID NO: 159 |
| AAV CKd-H6 | 766 | U.S. Pat. No. 8,734,809 SEQ ID NO: 151 |
| AAV CHt-1 | 767 | U.S. Pat. No. 8,734,809 SEQ ID NO: 160 |
| AAV CHt-P2 | 768 | WO2016065001 SEQ ID NO: 1 |
| AAV CHt-P5 | 769 | WO2016065001 SEQ ID NO: 2 |
| AAV CHt-P9 | 770 | WO2016065001 SEQ ID NO: 3 |
| AAV CBr-7.1 | 771 | WO2016065001 SEQ ID NO: 4 |
| AAV CBr-7.2 | 772 | WO2016065001 SEQ ID NO: 5 |
| AAV CBr-7.3 | 773 | WO2016065001 SEQ ID NO: 6 |
| AAV CBr-7.4 | 774 | WO2016065001 SEQ ID NO: 7 |
| AAV CBr-7.5 | 775 | WO2016065001 SEQ ID NO: 8 |
| AAV CBr-7.7 | 776 | WO2016065001 SEQ ID NO: 9 |
| AAV CBr-7.8 | 777 | WO2016065001 SEQ ID NO: 10 |
| AAV CBr-7.10 | 778 | WO2016065001 SEQ ID NO: 11 |
| AAV CKd-N3 | 779 | WO2016065001 SEQ ID NO: 12 |
| AAV CKd-N4 | 780 | WO2016065001 SEQ ID NO: 13 |
| AAV CKd-N9 | 781 | WO2016065001 SEQ ID NO: 14 |
| AAV CLv-L4 | 782 | WO2016065001 SEQ ID NO: 15 |
| AAV CLv-L5 | 783 | WO2016065001 SEQ ID NO: 16 |
| AAV CLv-L6 | 784 | WO2016065001 SEQ ID NO: 17 |
| AAV CLv-K1 | 785 | WO2016065001 SEQ ID NO: 18 |
| AAV CLv-K3 | 786 | WO2016065001 SEQ ID NO: 19 |
| AAV CLv-K6 | 787 | WO2016065001 SEQ ID NO: 20 |
| AAV CLv-M1 | 788 | WO2016065001 SEQ ID NO: 21 |
| AAV CLv-M11 | 789 | WO2016065001 SEQ ID NO: 22 |
| AAV CLv-M2 | 790 | WO2016065001 SEQ ID NO: 23 |
| AAV CLv-M5 | 791 | WO2016065001 SEQ ID NO: 24 |
| AAV CLv-M6 | 792 | WO2016065001 SEQ ID NO: 25 |
| AAV CLv-M7 | 793 | WO2016065001 SEQ ID NO: 26 |
| AAV CLv-M8 | 794 | WO2016065001 SEQ ID NO: 27 |
| AAV CLv-M9 | 795 | WO2016065001 SEQ ID NO: 28 |
| AAV CHt-P1 | 796 | WO2016065001 SEQ ID NO: 29 |
| AAV CHt-P6 | 797 | WO2016065001 SEQ ID NO: 30 |
| AAV CHt-P8 | 798 | WO2016065001 SEQ ID NO: 31 |
| AAV CHt-6.1 | 799 | WO2016065001 SEQ ID NO: 32 |
| AAV CHt-6.10 | 800 | WO2016065001 SEQ ID NO: 33 |
| AAV CHt-6.5 | 801 | WO2016065001 SEQ ID NO: 34 |
| AAV CHt-6.6 | 802 | WO2016065001 SEQ ID NO: 35 |
| AAV CHt-6.7 | 803 | WO2016065001 SEQ ID NO: 36 |
| AAV CHt-6.8 | 804 | WO2016065001 SEQ ID NO: 37 |
| AAV CSp-8.10 | 805 | WO2016065001 SEQ ID NO: 38 |
| AAV CSp-8.2 | 806 | WO2016065001 SEQ ID NO: 39 |
| AAV CSp-8.4 | 807 | WO2016065001 SEQ ID NO: 40 |
| AAV CSp-8.5 | 808 | WO2016065001 SEQ ID NO: 41 |
| AAV CSp-8.6 | 809 | WO2016065001 SEQ ID NO: 42 |
| AAV CSp-8.7 | 810 | WO2016065001 SEQ ID NO: 43 |
| AAV CSp-8.8 | 811 | WO2016065001 SEQ ID NO: 44 |
| AAV CSp-8.9 | 812 | WO2016065001 SEQ ID NO: 45 |
| AAV CBr-B7.3 | 813 | WO2016065001 SEQ ID NO: 46 |
| AAV CBr-B7.4 | 814 | WO2016065001 SEQ ID NO: 47 |
| AAV3B | 815 | WO2016065001 SEQ ID NO: 48 |
| AAV4 | 816 | WO2016065001 SEQ ID NO: 49 |
| AAV5 | 817 | WO2016065001 SEQ ID NO: 50 |
| AAV CHt-P2 | 818 | WO2016065001 SEQ ID NO: 51 |
| AAV CHt-P5 | 819 | WO2016065001 SEQ ID NO: 52 |
| AAV CHt-P9 | 820 | WO2016065001 SEQ ID NO: 53 |
| AAV CBr-7.1 | 821 | WO2016065001 SEQ ID NO: 54 |
| AAV CBr-7.2 | 822 | WO2016065001 SEQ ID NO: 55 |
| AAV CBr-7.3 | 823 | WO2016065001 SEQ ID NO: 56 |
| AAV CBr-7.4 | 824 | WO2016065001 SEQ ID NO: 57 |
| AAV CBr-7.5 | 825 | WO2016065001 SEQ ID NO: 58 |
| AAV CBr-7.7 | 826 | WO2016065001 SEQ ID NO: 59 |
| AAV CBr-7.8 | 827 | WO2016065001 SEQ ID NO: 60 |
| AAV CBr-7.10 | 828 | WO2016065001 SEQ ID NO: 61 |
| AAV CKd-N3 | 829 | WO2016065001 SEQ ID NO: 62 |
| AAV CKd-N4 | 830 | WO2016065001 SEQ ID NO: 63 |
| AAV CKd-N9 | 831 | WO2016065001 SEQ ID NO: 64 |
| AAV CLv-L4 | 832 | WO2016065001 SEQ ID NO: 65 |
| AAV CLv-L5 | 833 | WO2016065001 SEQ ID NO: 66 |
| AAV CLv-L6 | 834 | WO2016065001 SEQ ID NO: 67 |
| AAV CLv-K1 | 835 | WO2016065001 SEQ ID NO: 68 |
| AAV CLv-K3 | 836 | WO2016065001 SEQ ID NO: 69 |
| AAV CLv-K6 | 837 | WO2016065001 SEQ ID NO: 70 |
| AAV CL-M1 | 838 | WO2016065001 SEQ ID NO: 71 |
| AAV CLv-M11 | 839 | WO2016065001 SEQ ID NO: 72 |
| AAV CLv-M2 | 840 | WO2016065001 SEQ ID NO: 73 |
| AAV CLv-M5 | 841 | WO2016065001 SEQ ID NO: 74 |
| AAV CLv-M6 | 842 | WO2016065001 SEQ ID NO: 75 |
| AAV CLv-M7 | 843 | WO2016065001 SEQ ID NO: 76 |
| AAV CLv-M8 | 844 | WO2016065001 SEQ ID NO: 77 |
| AAV CLv-M9 | 845 | WO2016065001 SEQ ID NO: 78 |
| AAV CHt-P1 | 846 | WO2016065001 SEQ ID NO: 79 |
| AAV CHt-P6 | 847 | WO2016065001 SEQ ID NO: 80 |
| AAV CHt-P8 | 848 | WO2016065001 SEQ ID NO: 81 |
| AAV CHt-6.1 | 849 | WO2016065001 SEQ ID NO: 82 |
| AAV CHt-6.10 | 850 | WO2016065001 SEQ ID NO: 83 |
| AAV CHt-6.5 | 851 | WO2016065001 SEQ ID NO: 84 |
| AAV CHt-6.6 | 852 | WO2016065001 SEQ ID NO: 85 |
| AAV CHt-6.7 | 853 | WO2016065001 SEQ ID NO: 86 |
| AAV CHt-6.8 | 854 | WO2016065001 SEQ ID NO: 87 |
| AAV CSp-8.10 | 855 | WO2016065001 SEQ ID NO: 88 |
| AAV CSp-8.2 | 856 | WO2016065001 SEQ ID NO: 89 |
| AAV CSp-8.4 | 857 | WO2016065001 SEQ ID NO: 90 |
| AAVTSp-8.5 | 858 | WO2016065001 SEQ ID NO: 91 |
| AAV CSp-8.6 | 859 | WO2016065001 SEQ ID NO: 92 |
| AAV CSp-8.7 | 860 | WO2016065001 SEQ ID NO: 93 |
| AAV CSp-8.8 | 861 | WO2016065001 SEQ ID NO: 94 |
| AAV CSp-8.9 | 862 | WO2016065001 SEQ ID NO: 95 |
| AAV CBr-B7.3 | 863 | WO2016065001 SEQ ID NO: 96 |
| AAV CBr-B7.4 | 864 | WO2016065001 SEQ ID NO: 97 |
| AAV3B | 865 | WO2016065001 SEQ ID NO: 98 |
| AAV4 | 866 | WO2016065001 SEQ ID NO: 99 |
| AAV5 | 867 | WO2016065001 SEQ ID NO: 100 |
| AAVPHP.B | 868 | WO2015038958 SEQ ID NO: 8 and 13, |
| or G2B-26 | | GenBankALU85156.1 |
| AAVPHP.B | 869 | WO2015038958 SEQ ID NO: 9 |
| AAVG2B-13 | 870 | WO2015038958 SEQ ID NO: 12 |
| AAVTH1.1-32 | 871 | WO2015038958 SEQ ID NO: 14 |
| AAVTH1.1-35 | 872 | WO2015038958 SEQ ID NO: 15 |
| PHP.N/ | 1439 | WO2017100671 SEQ ID NO: 46 |
| PHP.B-DGT | | |
| PHP.S/G2A12 | 1440 | WO2017100671 SEQ ID NO: 47 |
| AAV9/hu.14 | 1441 | WO2017100671 SEQ ID NO: 45 |
| K449R | | |
| GPV | 1518 | U.S. Pat. No. 9,624,274B2 SEQ ID NO: 192 |
| B19 | 1519 | U.S. Pat. No. 9,624,274B2 SEQ ID NO: 193 |
| MVM | 1520 | U.S. Pat. No. 9,624,274B2 SEQ ID NO: 194 |
| FPV | 1521 | U.S. Pat. No. 9,624,274B2 SEQ ID NO: 195 |
| CPV | 1522 | U.S. Pat. No. 9,624,274B2 SEQ ID NO: 196 |
| AAV6 | 1523 | U.S. Pat. No. 9,546,112B2 SEQ ID NO: 5 |
| AAV6 | 1524 | U.S. Pat. No. 9,457,103B2 SEQ ID NO: 1 |
| AAV2 | 1525 | U.S. Pat. No. 9,457,103B2 SEQ ID NO: 2 |
| ShH10 | 1526 | U.S. Pat. No. 9,457,103B2 SEQ ID NO: 3 |
| ShH13 | 1527 | U.S. Pat. No. 9,457,103B2 SEQ ID NO: 4 |
| ShH10 | 1528 | U.S. Pat. No. 9,457,103B2 SEQ ID NO: 5 |
| ShH10 | 1529 | U.S. Pat. No. 9,457,103B2 SEQ ID NO: 6 |
| ShH10 | 1530 | U.S. Pat. No. 9,457,103B2 SEQ ID NO: 7 |
| ShH10 | 1531 | U.S. Pat. No. 9,457,103B2 SEQ ID NO: 8 |
| ShH10 | 1532 | U.S. Pat. No. 9,457,103B2 SEQ ID NO: 9 |
| rh74 | 1533 | U.S. Pat. No. 9,434,928B2 SEQ ID NO: 1, |
| | | US2015023924A1 SEQ ID NO: 2 |
| rh74 | 1534 | U.S. Pat. No. 9,434,928B2 SEQ ID NO: 2, |
| | | US2015023924A1 SEQ ID NO: 1 |
| AAV8 | 1535 | U.S. Pat. No. 9,434,928B2 SEQ ID NO: 4 |
| rh74 | 1536 | U.S. Pat. No. 9,434,928B2 SEQ ID NO: 5 |
| rh74 | 1537 | US2015023924A1 SEQ ID NO: 5, |
| (RHM4-1) | | US20160375110A1 SEQ ID NO: 4 |
| rh74 | 1538 | US2015023924A1 SEQ ID NO: 6, |
| (RHM15-1) | | US20160375110A1 SEQ ID NO: 5 |
| rh74 | 1539 | US2015023924A1 SEQ ID NO: 7, |
| (RHM15-2) | | US20160375110A1 SEQ ID NO: 6 |
| rh74 | 1540 | US2015023924A1 SEQ ID NO: 8, |
| (RHM15- | | US20160375110A1 SEQ ID NO: 7 |
| 3/RHM15-5) | | |
| rh74 | 1541 | US2015023924A1 SEQ ID NO: 9, |
| (RHM15-4) | | US20160375110A1 SEQ ID NO: 8 |
| rh74 | 1542 | US2015023924A1 SEQ ID NO: 10, |
| (RHM15-6) | | US20160375110A1 SEQ ID NO: 9 |
| rh74 | 1543 | US2015023924A1 SEQ ID NO: 11 |
| (RHM4-1) | | |
| rh74 | 1544 | US2015023924A1 SEQ ID NO: 12 |
| (RHM15-1) | | |
| rh74 | 1545 | US2015023924A1 SEQ ID NO: 13 |
| (RHM15-2) | | |
| rh74 | 1546 | US2015023924A1 SEQ ID NO: 14 |
| (RHM15- | | |
| 3/RHM15-5) | | |
| rh74 | 1547 | US2015023924A1 SEQ ID NO: 15 |
| (RHM15-4) | | |
| rh74 | 1548 | US2015023924A1 SEQ ID NO: 16 |
| (RHM15-6) | | |
| AAV2 | 1549 | US20160175389A1 SEQ ID NO: 9 |
| (comprising | | |
| lung specific | | |
| polypeptide) | | |
| AAV2 | 1550 | US20160175389A1 SEQ ID NO: 10 |
| (comprising | | |
| lung specific | | |
| polypeptide) | | |
| Anc80 | 1551 | US20170051257A1 SEQ ID NO: 1 |
| Anc80 | 1552 | US20170051257A1 SEQ ID NO: 2 |
| Anc81 | 1553 | US20170051257A1 SEQ ID NO: 3 |
| Anc80 | 1554 | US20170051257A1 SEQ ID NO: 4 |
| Anc82 | 1555 | US20170051257A1 SEQ ID NO: 5 |
| Anc82 | 1556 | US20170051257A1 SEQ ID NO: 6 |
| Anc83 | 1557 | US20170051257A1 SEQ ID NO: 7 |
| Anc83 | 1558 | US20170051257A1 SEQ ID NO: 8 |
| Anc84 | 1559 | US20170051257A1 SEQ ID NO: 9 |
| Anc84 | 1560 | US20170051257A1 SEQ ID NO: 10 |
| Anc94 | 1561 | US20170051257A1 SEQ ID NO: 11 |
| Anc94 | 1562 | US20170051257A1 SEQ ID NO: 12 |
| Anc113 | 1563 | US20170051257A1 SEQ ID NO: 13 |
| Anc113 | 1564 | US20170051257A1 SEQ ID NO: 14 |
| Anc126 | 1565 | US20170051257A1 SEQ ID NO: 15 |
| Anc126 | 1566 | US20170051257A1 SEQ ID NO: 16 |
| Anc127 | 1567 | US20170051257A1 SEQ ID NO: 17 |
| Anc127 | 1568 | US20170051257A1 SEQ ID NO: 18 |
| Anc80L27 | 1569 | US20170051257A1 SEQ ID NO: 19 |
| Anc80L59 | 1570 | US20170051257A1 SEQ ID NO: 20 |
| Anc80L60 | 1571 | US20170051257A1 SEQ ID NO: 21 |
| Anc80L62 | 1572 | US20170051257A1 SEQ ID NO: 22 |
| Anc80L65 | 1573 | US20170051257A1 SEQ ID NO: 23 |
| Anc80L33 | 1574 | US20170051257A1 SEQ ID NO: 24 |
| Anc80L36 | 1575 | US20170051257A1 SEQ ID NO: 25 |
| Anc80L44 | 1576 | US20170051257A1 SEQ ID NO: 26 |
| Anc80L1 | 1577 | US20170051257A1 SEQ ID NO: 35 |
| Anc80L1 | 1578 | US20170051257A1 SEQ ID NO: 36 |
| AAV-X1 | 1579 | U.S. Pat. No. 8,283,151B2 SEQ ID NO: 11 |
| AAV-X1b | 1580 | U.S. Pat. No. 8,283,151B2 SEQ ID NO: 12 |
| AAV-X5 | 1581 | U.S. Pat. No. 8,283,151B2 SEQ ID NO: 13 |
| AAV-X19 | 1582 | U.S. Pat. No. 8,283,151B2 SEQ ID NO: 14 |
| AAV-X21 | 1583 | U.S. Pat. No. 8,283,151B2 SEQ ID NO: 15 |
| AAV-X22 | 1584 | U.S. Pat. No. 8,283,151B2 SEQ ID NO: 16 |
| AAV-X23 | 1585 | U.S. Pat. No. 8,283,151B2 SEQ ID NO: 17 |
| AAV-X24 | 1586 | U.S. Pat. No. 8,283,151B2 SEQ ID NO: 18 |
| AAV-X25 | 1587 | U.S. Pat. No. 8,283,151B2 SEQ ID NO: 19 |
| AAV-X26 | 1588 | U.S. Pat. No. 8,283,151B2 SEQ ID NO: 20 |
| AAV-X1 | 1589 | U.S. Pat. No. 8,283,151B2 SEQ ID NO: 21 |
| AAV-X1b | 1590 | U.S. Pat. No. 8,283,151B2 SEQ ID NO: 22 |
| AAV-X5 | 1591 | U.S. Pat. No. 8,283,151B2 SEQ ID NO: 23 |
| AAV-X19 | 1592 | U.S. Pat. No. 8,283,151B2 SEQ ID NO: 24 |
| AAV-X21 | 1593 | U.S. Pat. No. 8,283,151B2 SEQ ID NO: 25 |
| AAV-X22 | 1594 | U.S. Pat. No. 8,283,151B2 SEQ ID NO: 26 |
| AAV-X23 | 1595 | U.S. Pat. No. 8,283,151B2 SEQ ID NO: 27 |
| AAV-X24 | 1596 | U.S. Pat. No. 8,283,151B2 SEQ ID NO: 28 |
| AAV-X25 | 1597 | U.S. Pat. No. 8,283,151B2 SEQ ID NO: 29 |
| AAV-X26 | 1598 | U.S. Pat. No. 8,283,151B2 SEQ ID NO: 30 |
| AAVrh8 | 1599 | WO2016054554A1 SEQ ID NO: 8 |
| AAVrh8VP2FC5 | 1600 | WO2016054554A1 SEQ ID NO: 9 |
| AAVrh8VP2FC44 | 1601 | WO2016054554A1 SEQ ID NO: 10 |
| AAVrh8VP2ApoB100 | 1602 | WO2016054554A1 SEQ ID NO: 11 |
| AAVrh8VP2RVG | 1603 | WO2016054554A1 SEQ ID NO: 12 |
| AAVrh8VP2Angiopep- | 1604 | WO2016054554A1 SEQ ID NO: 13 |
| 2 VP2 | | |
| AAV9.47VP1.3 | 1605 | WO2016054554A1 SEQ ID NO: 14 |
| AAV9.47VP2ICAMg3 | 1606 | WO2016054554A1 SEQ ID NO: 15 |
| AAV9.47VP2RVG | 1607 | WO2016054554A1 SEQ ID NO: 16 |
| AAV9.47VP2Angiopep- | 1608 | WO2016054554A1 SEQ ID NO: 17 |
| 2 | | |
| AAV9.47VP2A- | 1609 | WO2016054554A1 SEQ ID NO: 18 |
| string | | |
| AAVrh8VP2FC5 | 1610 | WO2016054554A1 SEQ ID NO: 19 |
| VP2 | | |
| AAVrh8VP2FC44 | 1611 | WO2016054554A1 SEQ ID NO: 20 |
| VP2 | | |
| AAVrh8VP2ApoB100 | 1612 | WO2016054554A1 SEQ ID NO: 21 |
| VP2 | | |
| AAVrh8VP2RVG | 1613 | WO2016054554A1 SEQ ID NO: 22 |
| VP2 | | |
| AAVrh8VP2Angiopep- | 1614 | WO2016054554A1 SEQ ID NO: 23 |
| 2 VP2 | | |
| AAV9.47VP2ICAMg3 | 1615 | WO2016054554A1 SEQ ID NO: 24 |
| VP2 | | |
| AAV9.47VP2RVG | 1616 | WO2016054554A1 SEQ ID NO: 25 |
| VP2 | | |
| AAV9.47VP2Angiopep- | 1617 | WO2016054554A1 SEQ ID NO: 26 |
| 2 VP2 | | |
| AAV9.47VP2A- | 1618 | WO2016054554A1 SEQ ID NO: 27 |
| string VP2 | | |
| rAAV-B1 | 1619 | WO2016054557A1 SEQ ID NO: 1 |
| rAAV-B2 | 1620 | WO2016054557A1 SEQ ID NO: 2 |
| rAAV-B3 | 1621 | WO2016054557A1 SEQ ID NO: 3 |
| rAAV-B4 | 1622 | WO2016054557A1 SEQ ID NO: 4 |
| rAAV-B1 | 1623 | WO2016054557A1 SEQ ID NO: 5 |
| rAAV-B2 | 1624 | WO2016054557A1 SEQ ID NO: 6 |
| rAAV-B3 | 1625 | WO2016054557A1 SEQ ID NO: 7 |
| rAAV-B4 | 1626 | WO2016054557A1 SEQ ID NO: 8 |
| rAAV-L1 | 1627 | WO2016054557A1 SEQ ID NO: 9 |
| rAAV-L2 | 1628 | WO2016054557A1 SEQ ID NO: 10 |
| rAAV-L3 | 1629 | WO2016054557A1 SEQ ID NO: 11 |
| rAAV-L4 | 1630 | WO2016054557A1 SEQ ID NO: 12 |
| rAAV-L1 | 1631 | WO2016054557A1 SEQ ID NO: 13 |
| rAAV-L2 | 1632 | WO2016054557A1 SEQ ID NO: 14 |
| rAAV-L3 | 1633 | WO2016054557A1 SEQ ID NO: 15 |
| rAAV-L4 | 1634 | WO2016054557A1 SEQ ID NO: 16 |
| AAV9 | 1635 | WO2016073739A1 SEQ ID NO: 3 |
| rAAV | 1636 | WO2016081811A1 SEQ ID NO: 1 |
| rAAV | 1637 | WO2016081811A1 SEQ ID NO: 2 |
| rAAV | 1638 | WO2016081811A1 SEQ ID NO: 3 |
| rAAV | 1639 | WO2016081811A1 SEQ ID NO: 4 |
| rAAV | 1640 | WO2016081811A1 SEQ ID NO: 5 |
| rAAV | 1641 | WO2016081811A1 SEQ ID NO: 6 |
| rAAV | 1642 | WO2016081811A1 SEQ ID NO: 7 |
| rAAV | 1643 | WO2016081811A1 SEQ ID NO: 8 |
| rAAV | 1644 | WO2016081811A1 SEQ ID NO: 9 |
| rAAV | 1645 | WO2016081811A1 SEQ ID NO: 10 |
| rAAV | 1646 | WO2016081811A1 SEQ ID NO: 11 |
| rAAV | 1647 | WO2016081811A1 SEQ ID NO: 12 |
| rAAV | 1648 | WO2016081811A1 SEQ ID NO: 13 |
| rAAV | 1649 | WO2016081811A1 SEQ ID NO: 14 |
| rAAV | 1650 | WO2016081811A1 SEQ ID NO: 15 |
| rAAV | 1651 | WO2016081811A1 SEQ ID NO: 16 |
| rAAV | 1652 | WO2016081811A1 SEQ ID NO: 17 |
| rAAV | 1653 | WO2016081811A1 SEQ ID NO: 18 |
| rAAV | 1654 | WO2016081811A1 SEQ ID NO: 19 |
| rAAV | 1655 | WO2016081811A1 SEQ ID NO: 20 |
| rAAV | 1656 | WO2016081811A1 SEQ ID NO: 21 |
| rAAV | 1657 | WO2016081811A1 SEQ ID NO: 22 |
| rAAV | 1658 | WO2016081811A1 SEQ ID NO: 23 |
| rAAV | 1659 | WO2016081811A1 SEQ ID NO: 24 |
| rAAV | 1660 | WO2016081811A1 SEQ ID NO: 25 |
| rAAV | 1661 | WO2016081811A1 SEQ ID NO: 26 |
| rAAV | 1662 | WO2016081811A1 SEQ ID NO: 27 |
| rAAV | 1663 | WO2016081811A1 SEQ ID NO: 28 |
| rAAV | 1664 | WO2016081811A1 SEQ ID NO: 29 |
| rAAV | 1665 | WO2016081811A1 SEQ ID NO: 30 |
| rAAV | 1666 | WO2016081811A1 SEQ ID NO: 31 |
| rAAV | 1667 | WO2016081811A1 SEQ ID NO: 32 |
| rAAV | 1668 | WO2016081811A1 SEQ ID NO: 33 |
| rAAV | 1669 | WO2016081811A1 SEQ ID NO: 34 |
| rAAV | 1670 | WO2016081811A1 SEQ ID NO: 35 |
| rAAV | 1671 | WO2016081811A1 SEQ ID NO: 36 |
| rAAV | 1672 | WO2016081811A1 SEQ ID NO: 37 |
| rAAV | 1673 | WO2016081811A1 SEQ ID NO: 38 |
| rAAV | 1674 | WO2016081811A1 SEQ ID NO: 39 |
| rAAV | 1675 | WO2016081811A1 SEQ ID NO: 40 |
| rAAV | 1676 | WO2016081811A1 SEQ ID NO: 41 |
| rAAV | 1677 | WO2016081811A1 SEQ ID NO: 42 |
| rAAV | 1678 | WO2016081811A1 SEQ ID NO: 43 |
| rAAV | 1679 | WO2016081811A1 SEQ ID NO: 44 |
| rAAV | 1680 | WO2016081811A1 SEQ ID NO: 45 |
| rAAV | 1681 | WO2016081811A1 SEQ ID NO: 46 |
| rAAV | 1682 | WO2016081811A1 SEQ ID NO: 47 |
| rAAV | 1683 | WO2016081811A1 SEQ ID NO: 48 |
| rAAV | 1684 | WO2016081811A1 SEQ ID NO: 49 |
| rAAV | 1685 | WO2016081811A1 SEQ ID NO: 50 |
| rAAV | 1686 | WO2016081811A1 SEQ ID NO: 51 |
| rAAV | 1687 | WO2016081811A1 SEQ ID NO: 52 |
| rAAV | 1688 | WO2016081811A1 SEQ ID NO: 53 |
| rAAV | 1689 | WO2016081811A1 SEQ ID NO: 54 |
| rAAV | 1690 | WO2016081811A1 SEQ ID NO: 55 |
| rAAV | 1691 | WO2016081811A1 SEQ ID NO: 56 |
| rAAV | 1692 | WO2016081811A1 SEQ ID NO: 57 |
| rAAV | 1693 | WO2016081811A1 SEQ ID NO: 58 |
| rAAV | 1694 | WO2016081811A1 SEQ ID NO: 59 |
| rAAV | 1695 | WO2016081811A1 SEQ ID NO: 60 |
| rAAV | 1696 | WO2016081811A1 SEQ ID NO: 61 |
| rAAV | 1697 | WO2016081811A1 SEQ ID NO: 62 |
| rAAV | 1698 | WO2016081811A1 SEQ ID NO: 63 |
| rAAV | 1699 | WO2016081811A1 SEQ ID NO: 64 |
| rAAV | 1700 | WO2016081811A1 SEQ ID NO: 65 |
| rAAV | 1701 | WO2016081811A1 SEQ ID NO: 66 |
| rAAV | 1702 | WO2016081811A1 SEQ ID NO: 67 |
| rAAV | 1703 | WO2016081811A1 SEQ ID NO: 68 |
| rAAV | 1704 | WO2016081811A1 SEQ ID NO: 69 |
| rAAV | 1705 | WO2016081811A1 SEQ ID NO: 70 |
| rAAV | 1706 | WO2016081811A1 SEQ ID NO: 71 |
| rAAV | 1707 | WO2016081811A1 SEQ ID NO: 72 |
| rAAV | 1708 | WO2016081811A1 SEQ ID NO: 73 |
| rAAV | 1709 | WO2016081811A1 SEQ ID NO: 74 |
| rAAV | 1710 | WO2016081811A1 SEQ ID NO: 75 |
| rAAV | 1711 | WO2016081811A1 SEQ ID NO: 76 |
| rAAV | 1712 | WO2016081811A1 SEQ ID NO: 77 |
| rAAV | 1713 | WO2016081811A1 SEQ ID NO: 78 |
| rAAV | 1714 | WO2016081811A1 SEQ ID NO: 79 |
| rAAV | 1715 | WO2016081811A1 SEQ ID NO: 80 |
| rAAV | 1716 | WO2016081811A1 SEQ ID NO: 81 |
| rAAV | 1717 | WO2016081811A1 SEQ ID NO: 82 |
| rAAV | 1718 | WO2016081811A1 SEQ ID NO: 83 |
| rAAV | 1719 | WO2016081811A1 SEQ ID NO: 84 |
| rAAV | 1720 | WO2016081811A1 SEQ ID NO: 85 |
| rAAV | 1721 | WO2016081811A1 SEQ ID NO: 86 |
| rAAV | 1722 | WO2016081811A1 SEQ ID NO: 87 |
| rAAV | 1723 | WO2016081811A1 SEQ ID NO: 88 |
| rAAV | 1724 | WO2016081811A1 SEQ ID NO: 89 |
| rAAV | 1725 | WO2016081811A1 SEQ ID NO: 90 |
| rAAV | 1726 | WO2016081811A1 SEQ ID NO: 91 |
| rAAV | 1727 | WO2016081811A1 SEQ ID NO: 92 |
| rAAV | 1728 | WO2016081811A1 SEQ ID NO: 93 |
| rAAV | 1729 | WO2016081811A1 SEQ ID NO: 94 |
| rAAV | 1730 | WO2016081811A1 SEQ ID NO: 95 |
| rAAV | 1731 | WO2016081811A1 SEQ ID NO: 96 |
| rAAV | 1732 | WO2016081811A1 SEQ ID NO: 97 |
| rAAV | 1733 | WO2016081811A1 SEQ ID NO: 98 |
| rAAV | 1734 | WO2016081811A1 SEQ ID NO: 99 |
| rAAV | 1735 | WO2016081811A1 SEQ ID NO: 100 |
| rAAV | 1736 | WO2016081811A1 SEQ ID NO: 101 |
| rAAV | 1737 | WO2016081811A1 SEQ ID NO: 102 |
| rAAV | 1738 | WO2016081811A1 SEQ ID NO: 103 |
| rAAV | 1739 | WO2016081811A1 SEQ ID NO: 104 |
| rAAV | 1740 | WO2016081811A1 SEQ ID NO: 105 |
| rAAV | 1741 | WO2016081811A1 SEQ ID NO: 106 |
| rAAV | 1742 | WO2016081811A1 SEQ ID NO: 107 |
| rAAV | 1743 | WO2016081811A1 SEQ ID NO: 108 |
| rAAV | 1744 | WO2016081811A1 SEQ ID NO: 109 |
| rAAV | 1745 | WO2016081811A1 SEQ ID NO: 110 |
| rAAV | 1746 | WO2016081811A1 SEQ ID NO: 111 |
| rAAV | 1747 | WO2016081811A1 SEQ ID NO: 112 |
| rAAV | 1748 | WO2016081811A1 SEQ ID NO: 113 |
| rAAV | 1749 | WO2016081811A1 SEQ ID NO: 114 |
| rAAV | 1750 | WO2016081811A1 SEQ ID NO: 115 |
| rAAV | 1751 | WO2016081811A1 SEQ ID NO: 116 |
| rAAV | 1752 | WO2016081811A1 SEQ ID NO: 117 |
| rAAV | 1753 | WO2016081811A1 SEQ ID NO: 118 |
| rAAV | 1754 | WO2016081811A1 SEQ ID NO: 119 |
| rAAV | 1755 | WO2016081811A1 SEQ ID NO: 120 |
| rAAV | 1756 | WO2016081811A1 SEQ ID NO: 121 |
| rAAV | 1757 | WO2016081811A1 SEQ ID NO: 122 |
| rAAV | 1758 | WO2016081811A1 SEQ ID NO: 123 |
| rAAV | 1759 | WO2016081811A1 SEQ ID NO: 124 |
| rAAV | 1760 | WO2016081811A1 SEQ ID NO: 125 |
| rAAV | 1761 | WO2016081811A1 SEQ ID NO: 126 |
| rAAV | 1762 | WO2016081811A1 SEQ ID NO: 127 |
| rAAV | 1763 | WO2016081811A1 SEQ ID NO: 128 |
| AAV8 | 1764 | WO2016081811A1 SEQ ID NO: 133 |
| E532K | | |
| AAV8 | 1765 | WO2016081811A1 SEQ ID NO: 134 |
| E532K | | |
| rAAV4 | 1766 | WO2016115382A1 SEQ ID NO: 2 |
| rAAV4 | 1767 | WO2016115382A1 SEQ ID NO: 3 |
| rAAV4 | 1768 | WO2016115382A1 SEQ ID NO: 4 |
| rAAV4 | 1769 | WO2016115382A1 SEQ ID NO: 5 |
| rAAV4 | 1770 | WO2016115382A1 SEQ ID NO: 6 |
| rAAV4 | 1771 | WO2016115382A1 SEQ ID NO: 7 |
| rAAV4 | 1772 | WO2016115382A1 SEQ ID NO: 8 |
| rAAV4 | 1773 | WO2016115382A1 SEQ ID NO: 9 |
| rAAV4 | 1774 | WO2016115382A1 SEQ ID NO: 10 |
| rAAV4 | 1775 | WO2016115382A1 SEQ ID NO: 11 |
| rAAV4 | 1776 | WO2016115382A1 SEQ ID NO: 12 |
| rAAV4 | 1777 | WO2016115382A1 SEQ ID NO: 13 |
| rAAV4 | 1778 | WO2016115382A1 SEQ ID NO: 14 |
| rAAV4 | 1779 | WO2016115382A1 SEQ ID NO: 15 |
| rAAV4 | 1780 | WO2016115382A1 SEQ ID NO: 16 |
| rAAV4 | 1781 | WO2016115382A1 SEQ ID NO: 17 |
| rAAV4 | 1782 | WO2016115382A1 SEQ ID NO: 18 |
| rAAV4 | 1783 | WO2016115382A1 SEQ ID NO: 19 |
| rAAV4 | 1784 | WO2016115382A1 SEQ ID NO: 20 |
| rAAV4 | 1785 | WO2016115382A1 SEQ ID NO: 21 |
| AAV11 | 1786 | WO2016115382A1 SEQ ID NO: 22 |
| AAV12 | 1787 | WO2016115382A1 SEQ ID NO: 23 |
| rh32 | 1788 | WO2016115382A1 SEQ ID NO: 25 |
| rh33 | 1789 | WO2016115382A1 SEQ ID NO: 26 |
| rh34 | 1790 | WO2016115382A1 SEQ ID NO: 27 |
| rAAV4 | 1791 | WO2016115382A1 SEQ ID NO: 28 |
| rAAV4 | 1792 | WO2016115382A1 SEQ ID NO: 29 |
| rAAV4 | 1793 | WO2016115382A1 SEQ ID NO: 30 |
| rAAV4 | 1794 | WO2016115382A1 SEQ ID NO: 31 |
| rAAV4 | 1795 | WO2016115382A1 SEQ ID NO: 32 |
| rAAV4 | 1796 | WO2016115382A1 SEQ ID NO: 33 |
| AAV2/8 | 1797 | WO2016115382A1 SEQ ID NO: 47 |
| AAV2/8 | 1798 | WO2016115382A1 SEQ ID NO: 48 |
| ancestral | 1799 | WO2016154344A1 SEQ ID NO: 7 |
| AAV | | |
| ancestral | 1800 | WO2016154344A1 SEQ ID NO: 13 |
| AAV variant | | |
| C4 | | |
| ancestral | 1801 | WO2016154344A1 SEQ ID NO: 14 |
| AAV variant | | |
| C7 | | |
| ancestral | 1802 | WO2016154344A1 SEQ ID NO: 15 |
| AAV variant | | |
| G4 | | |
| consensus | 1803 | WO2016154344A1 SEQ ID NO: 16 |
| amino acid | | |
| sequence of | | |
| ancestral | | |
| AAV | | |
| variants, C4, | | |
| C7 and G4 | | |
| consensus | 1804 | WO2016154344A1 SEQ ID NO: 17 |
| amino acid | | |
| sequence of | | |
| ancestral | | |
| AAV | | |
| variants, C4 | | |
| and C7 | | |
| AAV8 (with | 1805 | WO2016150403A1 SEQ ID NO: 13 |
| a AAV2 | | |
| phospholipase | | |
| domain) | | |
| AAV VR-942n | 1806 | US20160289275A1 SEQ ID NO: 10 |
| AAV5-A | 1807 | US20160289275A1 SEQ ID NO: 13 |
| (M569V) | | |
| AAV5-A | 1808 | US20160289275A1 SEQ ID NO: 14 |
| (M569V) | | |
| AAV5-A | 1809 | US20160289275A1 SEQ ID NO: 16 |
| (Y585V) | | |
| AAV5-A | 1810 | US20160289275A1 SEQ ID NO: 17 |
| (Y585V) | | |
| AAV5-A | 1811 | US20160289275A1 SEQ ID NO: 19 |
| (L587T) | | |
| AAV5-A | 1812 | US20160289275A1 SEQ ID NO: 20 |
| (L587T) | | |
| AAV5-A | 1813 | US20160289275A1 SEQ ID NO: 22 |
| (Y585V/ | | |
| L587T) | | |
| AAV5-A | 1814 | US20160289275A1 SEQ ID NO: 23 |
| (Y585V/ | | |
| L587T) | | |
| AAV5-B | 1815 | US20160289275A1 SEQ ID NO: 25 |
| (D652A) | | |
| AAV5-B | 1816 | US20160289275A1 SEQ ID NO: 26 |
| (D652A) | | |
| AAV5-B | 1817 | US20160289275A1 SEQ ID NO: 28 |
| (T362M) | | |
| AAV5-B | 1818 | US20160289275A1 SEQ ID NO: 29 |
| (T362M) | | |
| AAV5-B | 1819 | US20160289275A1 SEQ ID NO: 31 |
| (Q359D) | | |
| AAV5-B | 1820 | US20160289275A1 SEQ ID NO: 32 |
| (Q359D) | | |
| AAV5-B | 1821 | US20160289275A1 SEQ ID NO: 34 |
| (E350Q) | | |
| AAV5-B | 1822 | US20160289275A1 SEQ ID NO: 35 |
| (E350Q) | | |
| AAV5-B | 1823 | US20160289275A1 SEQ ID NO: 37 |
| (P533S) | | |
| AAV5-B | 1824 | US20160289275A1 SEQ ID NO: 38 |
| (P533S) | | |
| AAV5-B | 1825 | US20160289275A1 SEQ ID NO: 40 |
| (P533G) | | |
| AAV5B | 1826 | US20160289275A1 SEQ ID NO: 41 |
| (P533G) | | |
| AAV5- | 1827 | US20160289275A1 SEQ ID NO: 43 |
| mutation in | | |
| loop VII | | |
| AAV5- | 1828 | US20160289275A1 SEQ ID NO: 44 |
| mutation in | | |
| loop VII | | |
| AAV8 | 1829 | US20160289275A1 SEQ ID NO: 47 |
| Mut A | 1830 | WO2016181123A1 SEQ ID NO: 1 |
| (LK03/AAV8) | | |
| Mut B | 1831 | WO2016181123A1 SEQ ID NO: 2 |
| (LK03/AAV5) | | |
| Mut C | 1832 | WO2016181123A1 SEQ ID NO: 3 |
| (AAV8/AAV3B) | | |
| Mut D | 1833 | WO2016181123A1 SEQ ID NO: 4 |
| (AAV5/AAV3B) | | |
| Mut E | 1834 | WO2016181123A1 SEQ ID NO: 5 |
| (AAV8/AAV3B) | | |
| Mut F | 1835 | WO2016181123A1 SEQ ID NO: 6 |
| (AAV3B/AAV8) | | |
| AAV44.9 | 1836 | WO2016183297A1 SEQ ID NO: 4 |
| AAV44.9 | 1837 | WO2016183297A1 SEQ ID NO: 5 |
| AAVrh8 | 1838 | WO2016183297A1 SEQ ID NO: 6 |
| AAV44.9 | 1839 | WO2016183297A1 SEQ ID NO: 9 |
| (S470N) | | |
| rh74 VP1 | 1840 | US20160375110A1 SEQ ID NO: 1 |
| AAV-LK03 | 1841 | WO2017015102A1 SEQ ID NO: 5 |
| (L125I) | | |
| AAV3B | 1842 | WO2017015102A1 SEQ ID NO: 6 |
| (S663V + | | |
| T492V) | | |
| Anc80 | 1843 | WO2017019994A2 SEQ ID NO: 1 |
| Anc80 | 1844 | WO2017019994A2 SEQ ID NO: 2 |
| Anc81 | 1845 | WO2017019994A2 SEQ ID NO: 3 |
| Anc81 | 1846 | WO2017019994A2 SEQ ID NO: 4 |
| Anc82 | 1847 | WO2017019994A2 SEQ ID NO: 5 |
| Anc82 | 1848 | WO2017019994A2 SEQ ID NO: 6 |
| Anc83 | 1849 | WO2017019994A2 SEQ ID NO: 7 |
| Anc83 | 1850 | WO2017019994A2 SEQ ID NO: 8 |
| Anc84 | 1851 | WO2017019994A2 SEQ ID NO: 9 |
| Anc84 | 1852 | WO2017019994A2 SEQ ID NO: 10 |
| Anc94 | 1853 | WO2017019994A2 SEQ ID NO: 11 |
| Anc94 | 1854 | WO2017019994A2 SEQ ID NO: 12 |
| Anc113 | 1855 | WO2017019994A2 SEQ ID NO: 13 |
| Anc113 | 1856 | WO2017019994A2 SEQ ID NO: 14 |
| Anc126 | 1857 | WO2017019994A2 SEQ ID NO: 15 |
| Anc126 | 1858 | WO2017019994A2 SEQ ID NO: 16 |
| Anc127 | 1859 | WO2017019994A2 SEQ ID NO: 17 |
| Anc127 | 1860 | WO2017019994A2 SEQ ID NO: 18 |
| Anc80L27 | 1861 | WO2017019994A2 SEQ ID NO: 19 |
| Anc80L59 | 1862 | WO2017019994A2 SEQ ID NO: 20 |
| Anc80L60 | 1863 | WO2017019994A2 SEQ ID NO: 21 |
| Anc80L62 | 1864 | WO2017019994A2 SEQ ID NO: 22 |
| Anc80L65 | 1865 | WO2017019994A2 SEQ ID NO: 23 |
| Anc80L33 | 1866 | WO2017019994A2 SEQ ID NO: 24 |
| Anc80L36 | 1867 | WO2017019994A2 SEQ ID NO: 25 |
| Am80L44 | 1868 | WO2017019994A2 SEQ ID NO: 26 |
| Anc80L1 | 1869 | WO2017019994A2 SEQ ID NO: 35 |
| Anc80L1 | 1870 | WO2017019994A2 SEQ ID NO: 36 |
| AAVrh10 | 1871 | WO2017019994A2 SEQ ID NO: 41 |
| Anc110 | 1872 | WO2017019994A2 SEQ ID NO: 42 |
| Anc110 | 1873 | WO2017019994A2 SEQ ID NO: 43 |
| AAVrh32.33 | 1874 | WO2017019994A2 SEQ ID NO: 45 |
| AAVrh74 | 1875 | WO2017049031A1 SEQ ID NO: 1 |
| AAV2 | 1876 | WO2017053629A2 SEQ ID NO: 49 |
| AAV2 | 1877 | WO2017053629A2 SEQ ID NO: 50 |
| AAV2 | 1878 | WO2017053629A2 SEQ ID NO: 82 |
| Parvo-like | 1879 | WO2017070476A2 SEQ ID NO: 1 |
| virus | | |
| Parvo-like | 1880 | WO2017070476A2 SEQ ID NO: 2 |
| virus | | |
| Parvo-like | 1881 | WO2017070476A2 SEQ ID NO: 3 |
| virus | | |
| Parvo-like | 1882 | WO2017070476A2 SEQ ID NO: 4 |
| virus | | |
| Parvo-like | 1883 | WO2017070476A2 SEQ ID NO: 5 |
| virus | | |
| Parvo-like | 1884 | WO2017070476A2 SEQ ID NO: 6 |
| virus | | |
| AAVrh.10 | 1885 | WO2017070516A1 SEQ ID NO: 7 |
| AAVrh.10 | 1886 | WO2017070516A1 SEQ ID NO: 14 |
| AAV2tYF | 1887 | WO2017070491A1 SEQ ID NO: 1 |
| AAV-SPK | 1888 | WO2017075619A1 SEQ ID NO: 28 |
| AAV2.5 | 1889 | US20170128528A1 SEQ ID NO: 13 |
| AAV1.1 | 1890 | US20170128528A1 SEQ ID NO: 15 |
| AAV6.1 | 1891 | US20170128528A1 SEQ ID NO: 17 |
| AAV6.3.1 | 1892 | US20170128528A1 SEQ ID NO: 18 |
| AAV2i8 | 1893 | US20170128528A1 SEQ ID NO: 28 |
| AAV2i8 | 1894 | US20170128528A1 SEQ ID NO: 29 |
| ttAAV | 1895 | US20170128528A1 SEQ ID NO: 30 |
| ttAAV- | 1896 | U820170128528A1 SEQ ID NO: 32 |
| S312N | | |
| ttAAV- | 1897 | US20170128528A1 SEQ ID NO: 33 |
| S312N | | |
| AAV6 | 1898 | WO2016134337A1 SEQ ID NO: 24 |
| (Y705, Y731, | | |
| and T492) | | |
| AAV2 | 1899 | WO2016134375A1 SEQ ID NO: 9 |
| AAV2 | 1900 | WO2016134375A1 SEQ ID NO: 10 |

Each of the patents, applications and/or publications listed in Table 1 arc hereby incorporated by reference in their entirety.

in some embodiments, the AAV serotype may be, or may have a sequence as described in International Patent Publication WO2015038958, the contents of which are herein incorporated by reference in their entirety, such as, but not limited to, AAV9 (SEQ ID NO: 2 and 11 of WO2015038958 or SEQ ID NO: 127 and 126 respectively herein), PHP.B (SEQ ID NO: 8 and 9 of WO2015038958, herein SEQ ID NO: 868 and 869), G2B-13 (SEQ ID NO: 12 of WO2015038958, herein SEQ m NO: 870), G2B-26 (SEQ m NO: 13 of WO2015038958, herein SEQ ID NO: 868 and 869), TH1.1-32 (SEQ ID NO: 14 of WO2015038958, herein SEQ ID NO: 871), TH1.1-35 (SEQ ID NO: 15 of WO2015038958, herein SEQ ID NO: 872) or variants thereof. Further, any of the targeting peptides or amino acid inserts described in WO2015038958, may be inserted into any parent AAV serotype, such as, but not limited to, AAV9 (SEQ ID NO: 126 for the DNA sequence and SEQ ID NO: 127 for the amino acid sequence). In some embodiments, the amino acid insert is inserted between amino acids 586-592 of the parent AAV (e.g., AAV9). In another embodiment, the amino acid insert is inserted between amino acids 588-589 of the parent AAV sequence. The amino acid insert may be, but is not limited to, any of the following amino acid sequences, TLAVPFK (SEQ ID NO: 1 of WO2015038958; herein SEQ ID NO: 873), KFPVALT (SEQ ID NO: 3 of WO2015038958; herein SEQ m NO: 874), LAVPFK (SEQ ID NO: 31 of WO2015038958; herein SEQ m NO: 875), AVPFK (SEQ ID NO: 32 of WO2015038958; herein SEQ ID NO: 876), VPFK (SEQ ID NO: 33 of WO2015038958, herein SEQ ID NO: 877), TLAVPF (SEQ ID NO: 34 of WO2015038958; herein SEQ ID NO: 878), TLAVP (SEQ ID NO: 35 of WO2015038958; herein SEQ ID NO: 879), TLAV (SEQ ID NO: 36 of WO2015038958; herein SEQ ID NO: 880), SVSKPFL (SEQ ID NO: 28 of WO2015038958; herein SEQ ID NO: 881), FTLTTPK (SEQ ID NO: 29 of WO2015038958; herein SEQ ID NO: 882), MNATKNV (SEQ ID NO: 30 of WO2015038958; herein SEQ ID NO: 883), QSSQTPR (SEQ ID NO: 54 of WO2015038958; herein SEQ ID NO: 884), ILGTGTS (SEQ ID NO: 55 of WO2015038958; herein SEQ ID NO: 885), TRTNPEA (SEQ ID NO: 56 of WO2015038958; herein SEQ ID NO: 886), NGGTSSS (SEQ ID NO: 58 of WO2015038958; herein SEQ ID NO: 887), or YTLSQGW (SEQ NO: 60 of WO2015038958; herein SEQ ID NO: 888), Non-limiting examples of nucleotide sequences that may encode the amino acid inserts include the following, AAGTTTCCTGTGGCGTTGACT (for SEQ ID NO: 3 of WO2015038958; herein SEQ ID NO: 889), ACTTTGGCGGTGCCTTTTAAG (SEQ ID NO: 24 and 49 of WO2015038958, herein SEQ NO: 890), AGTGTGAGTAAGCCTTTTTTG (SEQ ID NO: 25 of WO2015038958; herein SEQ ID NO: 891), TTTACUITGACGACGCCTAAG (SEQ ID NO: 26 of WO2015038958; herein SEQ ID NO: 892), ATGAATGCTACGAAGAATGTG (SEQ ID NO: 27 of WO2015038958; herein SEQ ID NO: 893), CAGTCGTCGCAGACGCCTAGG (SEQ ID NO: 48 of WO2015038958; herein SEQ ID NO: 894), ATTCTGGGGACTGGTACTTCG (SEQ ID NO: 50 and 52 of WO2015038958, herein SEQ ID NO: 895), ACGCGGACTAATCCTGAGGCT (SEQ ID NO: 51 of WO2015038958; herein SEQ ID NO: 896), AATGGGGGGACTAGTAGTTCT (SEQ ID NO: 53 of WO2015038958; herein SEQ ID NO: 897), or TATACTTTGTCGCAGGGTTGG (SEQ ID NO: 59 of WO2015038958; herein SEQ ID NO: 898),

In some embodiments, the AAV serotype may be engineered to comprise at least one AAV capsid CD8+ T-cell epitope for AAV2 such as, but not limited to, SADNNNSEY (SEQ ID NO: 899), LIDQYLYYL (SEQ ID NO: 900), VPQYGYLTL (SEQ ID NO: 901), TTSTRTWAL (SEQ ID NO: 902), YHLNGRDSL (SEQ ID NO: 903), SQAVGRSSF (SEQ ID NO: 904), VPANPSTTF (SEQ ID NO: 905), FPQSGVLIF (SEQ ID NO: 906), YFDFNRFHCFSPRD (SEQ ID NO: 907), VGNSSGNWHCDSTWM (SEQ ID NO: 908). QFSQAGASDIRDQSR (SEQ ID NO: 909), GASDIRQSRNWLP (SEQ ID NO: 910) and GNRQAATADVNTQGV (SEQ ID NO: 911).

In some embodiments, the AAV serotype may be engineered to comprise at least one AAV capsid CD8+ T-cell epitope for AAV1. such as, but not limited to. LDRLIVINPLI (SEQ ID NO: 912), TTSTRTWAL (SEQ ID NO: 902), and QPAKKRLNF (SEQ ID NO: 913)).

In some embodiments, peptides for inclusion in an AAV serotype may be identified using the methods described by Hui et al. (Molecular Therapy—Methods & Clinical Development (2015) 2, 15029 doi:10.1038/mtm.2015.29: the contents of which are herein incorporated by reference in its entirety). As a non-limiting example, the procedure includes isolating human splenocytes, re-stimulating the splenocytes in vitro using individual peptides spanning the amino acid sequence of the AAV capsid protein, IFN-gamma ELISpot with the individual peptides used for the in vitro re-stimulation, bioinformatics analysis to determine the HLA restriction of 15-mers identified by IFN-gamma ELISpot, identification of candidate reactive 9-mer epitopes for a given HLA allele, synthesis candidate 9-mers, second WN-gamma ELISpot screening of splenocytes from subjects carrying the HLA alleles to which identified AAV epitopes are predicted to bind, determine the AAV capsid-reactive CD8+ T-cell epitopes and determine the frequency of subjects reacting to a given AAV epitope.

In some embodiments, the AAV may be a serotype generated by Cre-recombination-based AAV targeted evolution (CREATE) as described by Deverman et al., (Nature Biotechnology 34(2):204-209 (2016)), the contents of which are herein incorporated by reference in their entirety. In some embodiments, AAV serotypes generated in this manner have improved CNS transduction and/or neuronal and astrocytic tropism, as compared to other AAV serotypes. As non-limiting examples, the AAV serotype may be PHP.B, PHP.B2, PHP.B3, PHP.A, G2A12, G2A15. In some embodiments, these AAV serotypes may be AAV9 (SEQ ID NO: 126 and 127) derivatives with a 7-amino acid insert between amino acids 588-589. Non-limiting examples of these 7-amino acid inserts include TLAVPFK (SEQ ID NO: 873), SVSKPFL (SEQ ID NO: 881), FTLTTPK (SEQ ID NO: 882), YTLSQGW (SEQ ID NO: 888), QAVRTSL (SEQ NO: 1176) and/or LAKERLS (SEQ ID NO: 1177).

In some embodiments, the AAV serotype may be, or may have a sequence as described in International Patent Publication WO2017100671, the contents of which are herein incorporated by reference in their entirety, such as, but not limited to, AAV9 (SEQ ID NO: 45 of WO2017100671, herein SEQ ID NO: 1441), PHP.N (SEQ ID NO: 46 of WO2017100671, herein SEQ ID NO: 1439), PHP.S (SEQ ID NO: 47 of WO201.7100671, herein SEQ ID NO: 1440), or variants thereof. Further, any of the targeting peptides or amino acid inserts described in WO2017100671 may be inserted into any parent AAV serotype, such as, but not limited to, AAV9 (SEQ ID NO: 122 SEQ ID NO: 1441). In some embodiments, the amino acid insert is inserted between amino acids 586-592 of the parent AAV (e.g., AAV9). In another embodiment, the amino acid insert is inserted between amino acids 588-589 of the parent AAV sequence. The amino acid insert may be, but is not limited to, any of the following amino acid sequences, AQTLATPFKAQ (SEQ ID NO: 1 of WO2017100671; herein SEQ ID NO: 1442), AQSVSKPFLAQ (SEQ ID NO: 2 of WO2017100671; herein SEQ ID NO: 1443). AQFTLTTPKAQ (SEQ ID NO: 3 in the sequence listing of WO2017100671; herein SEQ ID NO: 1444), DGTLAVPFKAQ (SEQ ID NO: 4 in the sequence listing of WO2017100671; herein SEQ ID NO: 1445), ESTLAVPFKAQ (SEQ ID NO: 5 of WO2017100671; herein SEQ ID NO: 1446), GGTLAVPFKAQ (SEQ ID NO: 6 of WO2017100671; herein SEQ ID NO: 1447), AQTLATPFKAQ (SEQ ID NO: 7 and 33 of WO2017100671; herein SEQ ID NO: 1448), ATTLATPFKAQ (SEQ ID NO: 8 of WO2017100671; herein SEQ ID NO: 1449), DGTIATPFKAQ (SEQ ID NO: 9 of WO2017100671; herein SEQ ID NO: 1450), GGTLATPFKAQ (SEQ ID NO: 10 of WO2017100671; herein SEQ ID NO: 1451), SGSLAVPFKAQ (SEQ ID NO: 11 of WO2017100671; herein SEQ ID NO: 1452), AQTLAQPFKAQ (SEQ ID NO: 12 of WO2017100671; herein SEQ ID NO: 1453), AQTLQQPFKAQ (SEQ ID NO: 13 of WO201.7100671.; herein SEQ ID NO: 1454), AQTLSNPFKAQ (SEQ ID NO: 14 of WO2017100671; herein SEQ ID NO: 1455), AQTLAVPFSNP (SEQ ID NO: 15 of WO2017100671; herein SEQ ID NO: 1456), QUITAVPFKAQ (SEQ ID NO: 16 of WO2017100671; herein SEQ NO: 1457), NQTLAVPFKAQ (SEQ ID NO: 17 of WO2017100671; herein SEQ ID NO: 1458), EGSLAVPFKAQ (SEQ ID NO: 18 of WO2017100671; herein SEQ ID NO: 1459), SGNLAVPFKAQ (SEQ ID NO: 19 of WO2017100671; herein SEQ ID NO: 1460), EGTLAVPFKAQ (SEQ ID NO: 20 of WO2017100671; herein SEQ ID NO: 1461), DSTLAVPFKAQ (SEQ ID NO: 21 in Table 1 of WO2017100671; herein SEQ ID NO: 1462), AQTLATPFKAQ (SEQ ID NO: 22 of WO2017100671; herein SEQ ID NO: 1463), AQTLSTPFKAQ (SEQ ID NO: 23 of WO201.7100671.; herein SEQ ID NO: 1464), AQTLPQPFKAQ (SEQ ID NO: 24 and 32 of WO2017100671; herein SEQ ID NO: 1465), AQTLSQPFKAQ (SEQ ID NO: 25 of WO2017100671; herein SEQ ID NO: 1466), AQTLQLPFKAQ (SEQ ID NO: 26 of WO2017100671; herein SEQ ID NO: 1467), AQTLTMPFKAQ (SEQ ID NO: 27, and 34 of WO2017100671 and SEQ ID NO: 35 in the sequence listing of WO2017100671; herein SEQ ID NO: 1468), AQTLTTPFKAQ (SEQ ID NO: 28 of WO2017100671; herein SEQ ID NO: 1469), AQYTLSQGWAQ (SEQ ID NO: 29 of WO2017100671; herein SEQ ID NO: 1470), AQMNATKNVAQ (SEQ ID NO: 30 of WO2017100671; herein SEQ ID NO: 1471), AQVSGGHHSAQ (SEQ ID NO: 31 of WO2017100671; herein SEQ ID NO: 1472), AQTLTAPFKAQ (SEQ ID NO: 35 in Table 1 of WO2017100671; herein SEQ ID NO: 1473), AQTLSKPFKAQ (SEQ ID NO: 36 of WO2017100671; herein SEQ ID NO: 1474), QAVRTSL (SEQ ID NO: 37 of WO2017100671; herein SEQ ID NO: 1475), YTLSQGW (SEQ ID NO: 38 of WO2017100671; herein SEQ ID NO: 888), LAKERLS (SEQ ID NO: 39 of WO2017100671; herein SEQ ID NO: 1476), TLAVPFK (SEQ ID NO: 40 in the sequence listing of WO2017100671; herein SEQ ID NO: 873), SVSKPFL (SEQ ID NO: 41 of WO2017100671; herein SEQ ID NO: 881), FTLTTPK (SEQ ID NO: 42 of WO2017100671; herein SEQ ID NO: 882). MNSTKNV (SEQ ID NO: 43 of WO2017100671; herein SEQ ID NO:1477), VSGGHHS (SEQ ID NO: 44 of WO2017100671; herein SEQ ID NO: 1478), SAQTLAVPFKAQAQ (SEQ ID NO: 48 of WO2017100671; herein SEQ ID NO: 1479), SXXXLAVPFKAQAQ (SEQ ID NO: 49 of WO2017100671 wherein X may be any amino acid; herein SEQ NO: 1480). SAQXXXVPFKAQAQ (SEQ ID NO: 50 of WO2017100671 wherein X may be any amino acid; herein SEQ ID NO: 1481). SAQTLXXXFKAQAQ (SEQ ID NO: 51 of WO2017100671 wherein X may be any amino acid; herein SEQ ID NO: 1482), SAQTLAVXXXAQAQ (SEQ ID NO: 52 of WO2017100671 wherein X may be any amino acid; herein SEQ ID NO: 1483). SAQTLAVPFXXXAQ (SEQ NO: 53 of WO2017100671. wherein X may be any amino acid; herein SEQ ID NO: 1484), TNHQSAQ (SEQ ID NO: 65 of WO2017100671; herein SEQ ID NO: 1485), AQAQTGW (SEQ NO: 66 of WO2017100671; herein SEQ ID NO: 1486), DGTLATPFK (SEQ ID NO: 67 of WO2017100671; herein SEQ ID NO: 1487), DGTLATPFKXX (SEQ ID NO: 68 of WO2017100671 wherein X may be any amino acid; herein SEQ ID NO: 1488), LAVPFKAQ (SEQ ID NO: 80 of WO2017100671; herein SEQ ID NO: 1489), VPFKAQ (SEQ ID NO: 81 of WO2017100671; herein SEQ ID NO: 1490), FKAQ (SEQ ID NO: 82 of WO2017100671; herein SEQ ID NO: 1491), AQTLAV (SEQ ID NO: 83 of WO2017100671; herein SEQ ID NO: 1492). AQTLAVPF (SEQ ID NO: 84 of WO2017100671; herein SEQ ID NO: 1493), QAVR (SEQ ID NO: 85 of WO2017100671; herein SEQ ID NO: 1494), AVRT (SEQ ID NO: 86 of WO2017100671; herein SEQ ID NO: 1495), VRTS (SEQ ID NO: 87 of WO201.7100671.; herein SEQ ID NO: 1496), RTSL (SEQ ID NO: 88 of WO2017100671; herein SEQ ID NO: 1497), QAVRT (SEQ ID NO: 89 of WO2017100671; herein SEQ ID NO: 1498), AVRTS (SEQ NO: 90 of WO2017100671; herein SEQ ID NO: 1499), VRTSL (SEQ ID NO: 91 of WO2017100671; herein SEQ ID NO: 1500), QAVRTS (SEQ ID NO: 92 of WO2017100671; herein SEQ ID NO: 1501), or AVRTSL (SEQ ID NO: 93 of WO2017100671; herein SEQ ID NO: 1502).

Non-limiting examples of nucleotide sequences that may encode the amino acid inserts include the following, GATGGGACTTTGGCGGTGCCTTTTAAGGCACAG (SEQ ID NO: 54 of WO2017100671; herein SEQ ID NO: 1503); GATGGGACGTTGGCGGTGCCTTTTAAGGCACAG (SEQ ID NO: 55 of WO2017100671; herein SEQ ID NO: 1504), CAGGCGGTTAGGACGTCTTTG (SEQ ID NO: 56 of WO2017100671; herein SEQ ID NO: 1505), CAGGTCTTCACGGACTCAGACTATCAG (SEQ ID NO: 57 and 78 of WO2017100671; herein SEQ ID NO: 1506), CAAGTAAAACCTCTACAAATGTGGTAAAATCG (SEQ ID NO: 58 of WO2017100671; herein SEQ ID NO: 1507), ACTCATCGACCAATACTTGTACTATCTCTCTAGAAC (SEQ ID NO: 59 of WO2017100671; herein SEQ ID NO: 1508), GGAAGTATTCCTTGGTTTTGAACCCA (SEQ ID NO: 60 of WO2017100671; herein SEQ ID NO: 1509), GGTCGCGGTTCTTGTTTGTGGAT (SEQ ID NO: 61 of WO2017100671; herein SEQ ID NO: 1510), CGACCTTGAAGCGCATGAACTCCT (SEQ ID NO: 62 of WO2017100671; herein SEQ ID NO: 1511), GTATTCCTTGGTTTTGAACCCAACCGGTCTGCGCCTGTGCMNNMNNMNNMNNMNN MNNMNNTTGGGCACTCTGGTGGTTTGTC (SEQ ID NO: 63 of WO20171.00671 wherein N may be A, C, T, or G; herein SEQ ID NO: 1512), GTATTCCTTGGTTTTGAACCCAACCGGTCTGCGCMNNMNNMNNAAAAGGCACCGCC AAAGTTTG (SEQ ID NO: 69 of WO2017100671 wherein N may be A, C, T, or G; herein SEQ ID NO: 1513), GTATTCCTTGGTTTTGAACCCAACCGGTCTGCGCCTGTGCMNNMNNMNNCACCGCC AAAGTTTGGGCACT (SEQ ID NO: 70 of WO2017100671 wherein N may be A, C, T, or G; herein SEQ ID NO: 1514), GTATTCCTTGGTTTTGAACCCAACCGGTCTGCGCCTGTGCCTTAAAMNNMNNMNNC AAAGTTTGGGCACTCTGGTGG (SEQ ID NO: 71 of WO2017100671 wherein N may be A, C, T, or G; herein SEQ ID NO: 1515), GTATTCCTTGGTTTTGAACCCAACCGGTCTGCGCCTGTGCCTTAAAAGGCACMNNM NNMNNTTGGGCACTCTGGTGGTTTGTG (SEQ ID NO: 72 of WO2017100671 wherein N may be A, C, T, or G; herein SEQ ID NO: 1516), ACTTTGGCGGTGCCTTTTAAG (SEQ ID NO: 74 of WO2017100671; herein SEQ ID NO: 890), AGTGTGAGTAAGCCTTTTTTG (SEQ ID NO: 75 of WO2017100671; herein SEQ ID NO: 891), TTTACGTTGACGACGCCTAAG (SEQ ID NO: 76 of WO2017100671; herein SEQ ID NO: 892), TATACTTTGTCGCAGGGTTGG (SEQ ID NO: 77 of WO2017100671; herein SEQ ID NO: 898), or CTTGCGAAGGAGCGGCTTTCG (SEQ ID NO: 79 of WO2017100671; herein SEQ ID NO: 1517).

In some embodiments, the AAV serotype may be, or may have a sequence as described in U.S. Pat. No. 9,624,274, the contents of which are herein incorporated by reference in their entirety, such as, but not limited to, AAV1 (SEQ ID NO: 181 of U.S. Pat. No. 9,624,274), AAV6 (SEQ ID NO: 182 of U.S. Pat. No. 9,624,274), AAV2 (SEQ ID NO: 183 of U.S. Pat. No. 9,624,274), AAV3b (SEQ ID NO: 184 of U.S. Pat. No. 9,624,274), AAV7 (SEQ ID NO: 185 of U.S. Pat. No. 9,624,274), AAV8 (SEQ ID NO: 186 of U.S. Pat. No. 9,624,274), AAV10 (SEQ ID NO: 187 of U.S. Pat. No. 9,624,274), AAV4 (SEQ m NO: 188 of U.S. Pat. No. 9,624,274), AAV11 (SEQ ID NO: 189 of U.S. Pat. No. 9,624,274), bAAV (SEQ ID NO: 190 of U.S. Pat. No. 9,624,274), AAV5 (SEQ ID NO: 191 of U.S. Pat. No. 9,624,274), GPV (SEQ ID NO: 192 of U.S. Pat. No. 9,624,274; herein SEQ ID NO: 1518), B19 (SEQ ID NO: 193 of U.S. Pat. No. 9,624,274; herein SEQ ID NO: 1519), MVM (SEQ ID NO: 194 of U.S. Pat. No. 9,624,274; herein SEQ ID NO: 1520), FPV (SEQ ID NO: 195 of U.S. Pat. No. 9,624,274; herein SEQ ID NO: 1521), CPV (SEQ ID NO: 196 of U.S. Pat. No. 9,624,274, herein SEQ ID NO: 1522) or variants thereof. Further, any of the structural protein inserts described in U.S. Pat. No. 9,624,274, may be inserted into, but not limited to, 1-453 and 1-587 of any parent AAV serotype, such as, but not limited to, AAV2 (SEQ ID NO: 183 of U.S. Pat. No. 9,624,274). The amino acid insert may be, but is not limited to, any of the following amino acid sequences, VNLTWSRASG (SEQ ID NO: 50 of U.S. Pat. No. 9,624,274; herein SEQ ID NO: 1901), EFCINHRGYWVCGD (SEQ ID NO:55 of U.S. Pat. No. 9,624,274; herein SEQ ID NO: 1902), EDGQVMDVDLS (SEQ ID NO: 85 of U.S. Pat. No. 9,624,274; herein SEQ ID NO: 1903), EKQRNGTLT (SEQ ID NO: 86 of U.S. Pat. No. 9,624,274; herein SEQ ID NO: 1904), TYQCRVMPHLPRALMR (SEQ ID NO: 87 of U.S. Pat. No. 9,624,274; herein SEQ ID NO: 1905), RHSTTQPRKTKGSG (SEQ ID NO: 88 of U.S. Pat. No. 9,624,274, herein SEQ ID NO: 1906), DSNPRGVSAYLSR (SEQ ID NO: 89 of U.S. Pat. No. 9,624,274; herein SEQ ID NO: 1907), TITCLWDLAPSK (SEQ ID NO: 90 of U.S. Pat. No. 9,624,274; herein SEQ ID NO: 1908), KTKGSGFFVF (SEQ ID NO: 91 of U.S. Pat. No. 9,624,274, herein SEQ ID NO: 1909), THPHLPRALMRS (SEQ ID NO: 92 of U.S. Pat. No. 9,624,274; herein SEQ ID NO: 1910), GETYQCRVTHPHLPRALMRSTTK (SEQ ID NO: 93 of U.S. Pat. No. 9,624,274, herein SEQ ID NO: 1911), LPRALMRS (SEQ ID NO: 94 of U.S. Pat. No. 9,624,274; herein SEQ ID NO: 1912), INHRGYWV (SEQ ID NO: 95 of U.S. Pat. No. 9,624,274; herein SEQ ID NO: 1913), CDAGSVRTNAPD (SEQ ID NO: 60 of U.S. Pat. No. 9,624,274; herein SEQ ID NO: 1914), AKAVSNLTESRSESLQS (SEQ ID NO: 96 of U.S. Pat. No. 9,624,274; herein SEQ ID NO: 1915), SLTGDEFKKVLET (SEQ ID NO: 97 of U.S. Pat. No. 9,624,274; herein SEQ ID NO: 1916), REAVAYRFEED (SEQ ID NO: 98 of U.S. Pat. No. 9,624,274; herein SEQ ID NO: 1917), INPEIITLDG (SEQ ID NO: 99 of U.S. Pat. No. 9,624,274; herein SEQ m NO: 1918), DISVTGAPVITATYL (SEQ ID NO: 100 of U.S. Pat. No. 9,624,274; herein SEQ ID NO: 1919), DISVTGAPVITA (SEQ ID NO: 101 of U.S. Pat. No. 9,624,274; herein SEQ ID NO: 1920), PKTVSNLTESSSESVQS (SEQ ID NO: 102 of U.S. Pat. No. 9,624,274; herein SEQ ID NO: 1921), SLMGDEFKAVLET (SEQ ID NO: 103 of U.S. Pat. No. 9,624,274; herein SEQ ID NO: 1922), QHSVAYTFEED (SEQ ID NO: 104 of U.S. Pat. No. 9,624,274; herein SEQ ID NO: 1923), INPEIITRDG (SEQ ID NO: 105 of U.S. Pat. No. 9,624,274, herein SEQ ID NO: 1924), DISLTGDPVITASYL (SEQ ID NO: 106 of U.S. Pat. No. 9,624,274; herein SEQ ID NO: 1925), DISLTGDPVITA (SEQ ID NO: 107 of U.S. Pat. No. 9,624,274; herein SEQ ID NO: 1926), DQSIDFEIDSA (SEQ ID NO: 108 of U.S. Pat. No. 9,624,274; herein SEQ ID NO: 1927), KNVSEDLPLPTFSPTLLGDS (SEQ ID NO: 109 of U.S. Pat. No. 9,624,274; herein SEQ ID NO: 1928), KNVSEDLPLPT (SEQ ID NO: 110 of U.S. Pat. No. 9,624,274; herein SEQ ID NO: 1929), CDSGRVRTDAPD (SEQ ID NO: 111 of U.S. Pat. No. 9,624,274; herein SEQ ID NO: 1930), FPEHLLVDFLQSLS (SEQ ID NO: 112 of U.S. Pat. No. 9,624,274; herein SEQ NO: 1931), DAEFRHDSG (SEQ ID NO: 65 of U.S. Pat. No. 9,624,274; herein SEQ ID NO: 1932), HYAAAQWDFGNTMCQL (SEQ m NO: 113 of 1359,624,274; herein SEQ ID NO: 1933), YAAQWDFGNTMCQ (SEQ ID NO: 114 of U.S. Pat. No. 9,624,274; herein SEQ ID NO: 1934), RSQKEGLHYT (SEQ ID NO: 115 of U.S. Pat. No. 9,624,274; herein SEQ ID NO: 1935), SSRTPSDKPVAHWANPQAE (SEQ ID NO: 116 of U.S. Pat. No. 9,624,274; herein SEQ ID NO: 1936), SRTPSDKPVAHWANP (SEQ ID NO: 117 of 1359,624,274; herein SEQ ID NO: 1937), SSRTPSDKP (SEQ ID NO: 118 of U.S. Pat. No. 9,624,274; herein SEQ ID NO: 1938), NADGNVDYHMNSVP (SEQ ID NO: 119 of U.S. Pat. No. 9,624,274; herein SEQ ID NO: 1939), DGNVDYHMNSV (SEQ ID NO: 120 of U.S. Pat. No. 9,624,274; herein SEQ ID NO: 1940), RSFKEFLQSSLRALRQ (SEQ ID NO: 121 of U.S. Pat. No. 9,624,274; herein SEQ ID NO: 1941); FKEFLQSSLRA (SEQ ID NO: 122 of U.S. Pat. No. 9,624,274; herein SEQ ID NO: 1942), or QMWAPQWGPD (SEQ ID NO: 123 of U.S. Pat. No. 9,624,274; herein SEQ ID NO: 1943).

In some embodiments, the AAV serotype may be, or may have a sequence as described in U.S. Pat. No. 9,475,845, the contents of which are herein incorporated by reference in their entirety, such as, but not limited to, AAV capsid proteins comprising modification of one or more amino acids at amino acid positions 585 to 590 of the native AAV2 capsid protein. Further the modification may result in, but not limited to, the amino acid sequence RGNRQA (SEQ ID NO: 3 of U.S. Pat. No. 9,475,845; herein SEQ ID NO: 1944), SSSTDP (SEQ ID NO: 4 of U.S. Pat. No. 9,475,845; herein SEQ ID NO: 1945), SSNTAP (SEQ ID NO: 5 of U.S. Pat. No. 9,475,845; herein SEQ ID NO: 1946), SNSNLP (SEQ ID NO: 6 of U.S. Pat. No. 9,475,845; herein SEQ ID NO: 1947), SSTTAP (SEQ ID NO: 7 of U.S. Pat. No. 9,475,845; herein SEQ ID NO: 1948), AANTAA (SEQ ID NO: 8 of U.S. Pat. No. 9,475,845; herein SEQ ID NO: 1949), QQNTAP (SEQ ID NO: 9 of U.S. Pat. No. 9,475,845, herein SEQ ID NO: 1950), SAQAQA (SEQ ID NO: 10 of U.S. Pat. No. 9,475,845; herein SEQ ID NO: 1951), QANTGP (SEQ ID NO: 11 of U.S. Pat. No. 9,475,845; herein SEQ ID NO: 1952), NATTAP (SEQ ID NO: 12 of U.S. Pat. No. 9,475,845; herein SEQ ID NO: 1953), SSTAGP (SEQ ID NO: 13 and 20 of U.S. Pat. No. 9,475,845; herein SEQ ID NO: 1954), QQNTAA (SEQ ID NO: 14 of U.S. Pat. No. 9,475,845; herein SEQ ID NO: 1955), PSTAGP (SEQ ID NO: 15 of U.S. Pat. No. 9,475,845; herein SEQ ID NO: 1956). NQNTAP (SEQ ID NO: 16 of U.S. Pat. No. 9,475,845; herein SEQ ID NO: 1957), QAANAP (SEQ ID NO: 17 of U.S. Pat. No. 9,475,845; herein SEQ ID NO: 1958), SIVGLP (SEQ ID NO: 18 of U.S. Pat. No. 9,475,845; herein SEQ ID NO: 1959). AASTAA (SEQ ID NO: 19, and 27 of U.S. Pat. No. 9,475,845; herein SEQ ID NO: 1960), SQNTTA (SEQ ID NO: 21 of U.S. Pat. No. 9,475,845; herein SEQ NO: 1961), QQDTAP (SEQ ID NO: 22 of U.S. Pat. No. 9,475,845; herein SEQ ID NO: 1962), QTNTGP (SEQ ID NO: 23 of U.S. Pat. No. 9,475,845; herein SEQ ID NO: 1963), QTNGAP (SEQ ID NO: 24 of U.S. Pat. No. 9,475,845; herein SEQ ID NO: 1964), QQNAAP (SEQ ID NO: 25 of U.S. Pat. No. 9,475,845; herein SEQ ID NO: 1965), or AANTQA (SEQ ID NO: 26 of U.S. Pat. No. 9,475,845; herein SEQ NO: 1966). In some embodiments, the amino acid modification is a substitution at amino acid positions 262 through 265 in the native AAV2 capsid protein or the corresponding position in the capsid protein of another AAV with a targeting sequence. The targeting sequence may be, but is not limited to, any of the amino acid sequences, NGRAHA (SEQ ID NO: 38 of U.S. Pat. No. 9,475,845; herein SEQ ID NO: 1967). QPEHSST (SEQ ID NO: 39 and 50 of U.S. Pat. No. 9,475,845; herein SEQ ID NO: 1968), VNTANST (SEQ ID NO: 40 of U.S. Pat. No. 9,475,845; herein SEQ ID NO: 1969), HGPMQKS (SEQ ID NO: 41 of U.S. Pat. No. 9,475,845; herein SEQ ID NO: 1970), PHKPPILA (SEQ ID NO: 42 of U.S. Pat. No. 9,475,845; herein SEQ ID NO: 1971), IKNNEMW (SEQ ID NO: 43 of U.S. Pat. No. 9,475,845; herein SEQ ID NO: 1972), RNLDTPM (SEQ ID NO: 44 of U.S. Pat. No. 9,475,845; herein SEQ ID NO: 1973). VDSHRQS (SEQ ID NO: 45 of U.S. Pat. No. 9,475,845; herein SEQ ID NO: 1974), YDSKTKT (SEQ ID NO: 46 of U.S. Pat. No. 9,475,845; herein SEQ ID NO: 1975), SQLPHQK (SEQ ID NO: 47 of U.S. Pat. No. 9,475,845; herein SEQ NO: 1976). STMQQN17 (SEQ ID NO: 48 of U.S. Pat. No. 9,475,845; herein SEQ ID NO: 1977), TERYMTQ (SEQ ID NO: 49 of U.S. Pat. No. 9,475,845; herein SEQ ID NO: 1978), DASLSTS (SEQ ID NO: 51 of U.S. Pat. No. 9,475,845; herein SEQ ID NO: 1979), DLPNKKT (SEQ ID NO: 52 of U.S. Pat. No. 9,475,845; herein SEQ ID NO: 1980), DLTAARL (SEQ ID NO: 53 of U.S. Pat. No. 9,475,845; herein SEQ ID NO: 1981), EPHQFNY (SEQ ID NO: 54 of U.S. Pat. No. 9,475,845; herein SEQ ID NO: 1982). EPQSNHT (SEQ ID NO: 55 of U.S. Pat. No. 9,475,845; herein SEQ ID NO: 1983), MSSWPSQ (SEQ ID NO: 56 of U.S. Pat. No. 9,475,845; herein SEQ ID NO: 1984), NPKHNAT (SEQ ID NO: 57 of U.S. Pat. No. 9,475,845; herein SEQ ID NO: 1985). PDGMRTT (SEQ ID NO: 58 of U.S. Pat. No. 9,475,845; herein SEQ ID NO: 1986). PNNNKTT (SEQ ID NO: 59 of U.S. Pat. No. 9,475,845; herein SEQ ID NO: 1987), QSTTHDS (SEQ ID NO: 60 of U.S. Pat. No. 9,475,845; herein SEQ ID NO: 1988), TGSKQKQ (SEQ ID NO: 61 of U.S. Pat. No. 9,475,845; herein SEQ ID NO: 1989), SLKHQAL (SEQ ID NO: 62 of U.S. Pat. No. 9,475,845; herein SEQ ID NO: 1990), SPIDGEQ (SEQ NO: 63 of U.S. Pat. No. 9,475,845; herein SEQ ID NO: 1991), WIFPWIQL (SEQ ID NO: 64 and 112 of U.S. Pat. No. 9,475,845; herein SEQ ID NO: 1992), CDCRGDCFC (SEQ ID NO: 65 of U.S. Pat. No. 9,475,845; herein SEQ ID NO: 1993), CNGRC (SEQ ID NO: 66 of U.S. Pat. No. 9,475,845; herein SEQ ID NO: 1994), CPRECES (SEQ ID NO: 67 of U.S. Pat. No. 9,475,845; herein SEQ ID NO: 1995), CTTHWGFTLC (SEQ ID NO: 68 and 123 of U.S. Pat. No. 9,475,845; herein SEQ ID NO: 1996), CGRRAGGSC (SEQ ID NO: 69 of U.S. Pat. No. 9,475,845; herein SEQ m NO: 1997), CKGGRAKDC (SEQ ID NO: 70 of U.S. Pat. No. 9,475,845; herein SEQ ID NO: 1998), CVPELGHEC (SEQ ID NO: 71 and 115 of U.S. Pat. No. 9,475,845; herein SEQ ID NO: 1999), CRRETAWAK (SEQ ID NO: 72 of U.S. Pat. No. 9,475,845; herein SEQ ID NO: 2000), VSWFSHRYSPFAVS (SEQ ID NO: 73 of U.S. Pat. No. 9,475,845; herein SEQ ID NO: 2001), GYRDGYAGPILYN (SEQ ID NO: 74 of U.S. Pat. No. 9,475,845; herein SEQ ID NO: 2002), XXXYXXX (SEQ ID NO: 75 of U.S. Pat. No. 9,475,845, herein SEQ ID NO: 2003), YXNW (SEQ ID NO: 76 of U.S. Pat. No. 9,475,845; herein SEQ ID NO: 2004), RPLPPLP (SEQ ID NO: 77 of U.S. Pat. No. 9,475,845; herein SEQ ID NO: 2005), APPLPPR (SEQ ID NO: 78 of U.S. Pat. No. 9,475,845; herein SEQ ID NO: 2006), DVFYPYPYASGS (SEQ ID NO: 79 of U.S. Pat. No. 9,475,845; herein SEQ ID NO: 2007). MYWYPY (SEQ ID NO: 80 of U.S. Pat. No. 9,475,845; herein SEQ ID NO: 2008), DITWDQLWDLMK (SEQ ID NO: 81 of U.S. Pat. No. 9,475,845; herein SEQ ID NO: 2009), CWDDXWLC (SEQ ID NO: 82 of U.S. Pat. No. 9,475,845; herein SEQ ID NO: 2010), EWCEYLGGYLRCYA (SEQ ID NO: 83 of U.S. Pat. No. 9,475,845; herein SEQ ID NO: 2011), YXCXXGPXTWXCXP (SEQ ID NO: 84 of U.S. Pat. No. 9,475,845; herein SEQ ID NO: 2012), IEGVITRQWLAARA (SEQ ID NO: 85 of U.S. Pat. No. 9,475,845; herein SEQ ID NO: 2013), LWXXX (SEQ ID NO: 86 of U.S. Pat. No. 9,475,845; herein SEQ ID NO: 2014), XFXXYLW (SEQ ID NO: 87 of U.S. Pat. No. 9,475,845; herein SEQ ID NO: 2015). SSIISHFRWGLCD (SEQ ID NO: 88 of U.S. Pat. No. 9,475,845; herein SEQ ID NO: 2016), MSRPACPPNDKYE (SEQ ID NO: 89 of U.S. Pat. No. 9,475,845; herein SEQ ID NO: 2017), CLRSGRGC (SEQ ID NO: 90 of U.S. Pat. No. 9,475,845; herein SEQ ID NO: 2018), CHWMFSPWC (SEQ m NO: 91 of U.S. Pat. No. 9,475,845; herein SEQ ID NO: 2019), WXXF (SEQ ID NO: 92 of U.S. Pat. No. 9,475,845, herein SEQ ID NO: 2020). CSSRLDAC (SEQ ID NO: 93 of U.S. Pat. No. 9,475,845; herein SEQ ID NO: 2021), CLPVASC (SEQ ID NO: 94 of U.S. Pat. No. 9,475,845; herein SEQ ID NO: 2022), CGFECVRQCPERC (SEQ ID NO: 95 of U.S. Pat. No. 9,475,845; herein SEQ ID NO: 2023), CVALCREACGEGC (SEQ ID NO: 96 of U.S. Pat. No. 9,475,845; herein SEQ ID NO: 2024), SWCEPGWCR (SEQ ID NO: 97 of U.S. Pat. No. 9,475,845; herein SEQ ID NO: 2025), YSGKWGW (SEQ ID NO: 98 of U.S. Pat. No. 9,475,845; herein SEQ ID NO: 2026). GLSGGRS (SEQ ID NO: 99 of U.S. Pat. No. 9,475,845; herein SEQ ID NO: 2027), LMLPRAD (SEQ ID NO: 100 of U.S. Pat. No. 9,475,845; herein SEQ ID NO: 2028), CSCFRDVCC (SEQ ID NO: 101 of U.S. Pat. No. 9,475,845; herein SEQ ID NO: 2029), CRDVVSVIC (SEQ ID NO: 102 of U.S. Pat. No. 9,475,845; herein SEQ ID NO: 2030), MARSGL (SEQ ID NO: 103 of U.S. Pat. No. 9,475,845; herein SEQ ID NO: 2031), MARAKE (SEQ ID NO: 104 of U.S. Pat. No. 9,475,845; herein SEQ ID NO: 2032), MSRTMS (SEQ ID NO: 105 of U.S. Pat. No. 9,475,845, herein SEQ ID NO: 033), KCCYSL (SEQ ID NO: 106 of U.S. Pat. No. 9,475,845; herein SEQ ID NO: 2034), MYWGDSHWLQYWYE (SEQ ID NO: 107 of U.S. Pat. No. 9,475,845; herein SEQ ID NO: 2035), MQLPLAT (SEQ ID NO: 108 of U.S. Pat. No. 9,475,845; herein SEQ ID NO: 2036), EWLS (SEQ ID NO: 109 of U.S. Pat. No. 9,475,845; herein SEQ ID NO: 2037), SNEW (SEQ ID NO: 110 of U.S. Pat. No. 9,475,845, herein SEQ ID NO: 2038), TNYL (SEQ ID NO: 111 of U.S. Pat. No. 9,475,845; herein SEQ ID NO: 2039), WDLAWMFRLPVG (SEQ ID NO: 113 of U.S. Pat. No. 9,475,845; herein SEQ ID NO: 2040), CTVALPGGYVRVC (SEQ ID NO: 114 of U.S. Pat. No. 9,475,845, herein SEQ ID NO: 2041), CVAYCIEHHCWTC (SEQ ID NO: 116 of U.S. Pat. No. 9,475,845; herein SEQ m NO: 2042), CVFAHNYDYLVC (SEQ ID NO: 117 of U.S. Pat. No. 9,475,845; herein SEQ ID NO: 2043), CVFTSNYAFC (SEQ ID NO: 118 of U.S. Pat. No. 9,475,845; herein SEQ ID NO: 2044), VHSPNKK (SEQ ID NO: 119 of U.S. Pat. No. 9,475,845; herein SEQ ID NO: 2045), CRGDGWC (SEQ ID NO: 120 of U.S. Pat. No. 9,475,845; herein SEQ ID NO: 2046), XRGCDX (SEQ ID NO: 121 of U.S. Pat. No. 9,475,845; herein SEQ ID NO: 2047), PXXX (SEQ ID NO: 122 of U.S. Pat. No. 9,475,845; herein SEQ ID NO: 2048), SGKGPRQITAL (SEQ ID NO: 124 of U.S. Pat. No. 9,475,845; herein SEQ ID NO: 2049), AAAAAAAAAXXXXX (SEQ NO: 125 of U.S. Pat. No. 9,475,845; herein SEQ ID NO: 2050), VYMSPF (SEQ ID NO: 126 of U.S. Pat. No. 9,475,845, herein SEQ ID NO: 2051), ATWLPPR (SEQ ID NO: 127 of U.S. Pat. No. 9,475,845; herein SEQ ID NO: 2052), HTMYYHHYQHHL (SEQ ID NO: 128 of U.S. Pat. No. 9,475,845; herein SEQ ID NO: 2053), SEVGCRAGPLQWLCEKYFG (SEQ ID NO: 129 of U.S. Pat. No. 9,475,845; herein SEQ ID NO: 2054), CGLLPVGRPDRNVWRWLC (SEQ ID NO: 130 of U.S. Pat. No. 9,475,845; herein SEQ ID NO: 2055), CKGQCDRFKGLPWEC (SEQ ID NO: 131 of U.S. Pat. No. 9,475,845; herein SEQ ID NO: 2056), SGRSA (SEQ ID NO: 132 of U.S. Pat. No. 9,475,845; herein SEQ ID NO: 2057), WGFP (SEQ NO: 133 of U.S. Pat. No. 9,475,845; herein SEQ ID NO: 2058), AEPMPHSLNFSQYLWYT (SEQ ID NO: 134 of U.S. Pat. No. 9,475,845; herein SEQ ID NO: 2059), WAYXSP (SEQ ID NO: 135 of U.S. Pat. No. 9,475,845; herein SEQ ID NO: 2060), IELLQAR (SEQ ID NO: 136 of U.S. Pat. No. 9,475,845; herein SEQ ID NO: 2061), AYTKCSRQWRTCMTTH (SEQ ID NO: 137 of U.S. Pat. No. 9,475,845; herein SEQ m NO: 2062), PQNSKIPGPTFLDPH (SEQ ID NO: 138 of U.S. Pat. No. 9,475,845; herein SEQ ID NO: 2063), SMEPALPDWWWKMFK (SEQ ID NO: 139 of U.S. Pat. No. 9,475,845; herein SEQ ID NO: 2064), ANTPCGPYTHDCPVKR (SEQ ID NO: 140 of U.S. Pat. No. 9,475,845, herein SEQ ID NO: 2065), TACHQHVRMVRP (SEQ ID NO: 141 of U.S. Pat. No. 9,475,845, herein SEQ m NO: 2066), VPWMEPAYQRFL (SEQ ID NO: 142 of U.S. Pat. No. 9,475,845; herein SEQ ID NO: 2067), DPRATPGS (SEQ ID NO: 143 of U.S. Pat. No. 9,475,845; herein SEQ ID NO: 2068), FRPNRAQDYNTN (SEQ ID NO: 144 of U.S. Pat. No. 9,475,845, herein SEQ ID NO: 2069), CTKNSYLMC (SEQ ID NO: 145 of U.S. Pat. No. 9,475,845; herein SEQ ID NO: 2070), CXXTXXXGXGC (SEQ ID NO: 146 of U.S. Pat. No. 9,475,845; herein SEQ ID NO: 2071), CPIEDRPMC (SEQ ID NO: 147 of U.S. Pat. No. 9,475,845; herein SEQ ID NO: 2072). HEWSYLAPYPWF (SEQ ID NO: 148 of U.S. Pat. No. 9,475,845; herein SEQ ID NO: 2073), MCPKHPLGC (SEQ ID NO: 149 of U.S. Pat. No. 9,475,845; herein SEQ ID NO: 2074), RMWPSSTVNLSAGRR (SEQ ID NO: 150 of U.S. Pat. No. 9,475,845; herein SEQ ID NO: 2075), SAKTAVSQRVWLPSHRGGEP (SEQ ID NO: 151 of U.S. Pat. No. 9,475,845; herein SEQ ID NO: 2076), KSREHVNNSACPSKRITAAL (SEQ ID NO: 152 of U.S. Pat. No. 9,475,845; herein SEQ ID NO: 2077), EGFR (SEQ ID NO: 153 of U.S. Pat. No. 9,475,845; herein SEQ ID NO: 2078), AGLGVR (SEQ ID NO: 154 of U.S. Pat. No. 9,475,845, herein SEQ ID NO: 2079), GTRQGHTMRLGVSDG (SEQ m NO: 155 of U.S. Pat. No. 9,475,845; herein SEQ ID NO: 2080), IAGLATPGWSHWLAL (SEQ ID NO: 156 of U.S. Pat. No. 9,475,845; herein SEQ ID NO: 2081), SMSIARL (SEQ ID NO: 157 of U.S. Pat. No. 9,475,845, herein SEQ ID NO: 2082), HTFEPGV (SEQ ID NO: 158 of U.S. Pat. No. 9,475,845; herein SEQ ID NO: 2083), NTSLKRISNKRIRRK (SEQ ID NO: 159 of U.S. Pat. No. 9,475,845; herein SEQ ID NO: 2084), LRIKRKRRKRKKTRK (SEQ ID NO: 160 of U.S. Pat. No. 9,475,845; herein SEQ ID NO: 2085), GGG, GES, LWS, EGG, LLV, LSP, LBS, AGG, GRR, GGH and GTV.

In some embodiments, the AAV serotype may be, or may have a sequence as described in United States Publication No. US 20160369298, the contents of which are herein incorporated by reference in their entirety, such as, but not limited to, site-specific mutated capsid protein of AAV2 (SEQ ID NO: 97 of US 20160369298; herein SEQ ID NO: 2086) or variants thereof, wherein the specific site is at least one site selected from sites R447, G453, S578, N587, N587+1, 5662 of VP1 or fragment thereof.

Further, any of the mutated sequences described in US 20160369298, may be or may have, but not limited to, any of the following sequences SDSGASN (SEQ ID NO: 1 and SEQ NO: 231 of US20160369298; herein SEQ ID NO: 2087), SPSGASN (SEQ ID NO: 2 of US20160369298; herein SEQ ID NO: 2088), SHSGASN (SEQ ID NO: 3 of US20160369298; herein SEQ NO: 2089), SRSGASN (SEQ ID NO: 4 of US20160369298; herein SEQ ID NO: 2090), SKSGASN (SEQ ID NO: 5 of US20160369298; herein SEQ ID NO: 2091), SNSGASN (SEQ ID NO: 6 of US20160369298; herein SEQ ID NO: 2092), SGSGASN (SEQ ID NO: 7 of US20160369298; herein SEQ ID NO: 2093), SASGASN (SEQ ID NO: 8, 175, and 221 of US20160369298; herein SEQ ID NO: 2094), SESGTSN (SEQ ID NO: 9 of US20160369298; herein SEQ m NO: 2095), STTGGSN (SEQ ID NO: 10 of US20160369298; herein SEQ NO: 2096), SSAGSTN (SEQ ID NO: 11 of US20160369298; herein SEQ ID NO: 2097), NNDSQA (SEQ ID NO: 12 of US20160369298; herein SEQ ID NO: 2098), NNRNQA (SEQ ID NO: 13 of US20160369298; herein SEQ ID NO: 2099), NNNKQA (SEQ ID NO: 14 of US20160369298; herein SEQ ID NO: 2100), NAKRQA (SEQ ID NO: 15 of US20160369298; herein SEQ ID NO: 2101), NDEHQA (SEQ ID NO: 16 of US20160369298; herein SEQ ID NO: 2102), NTSQKA (SEQ ID NO: 17 of US20160369298; herein SEQ ID NO: 2103), YYLSRTNTPSGTDTQSRLVFSQAGA (SEQ ID NO: 18 of US20160369298; herein SEQ ID NO: 2104), YYLSRTNTDSGTEFQSGLDFSQAGA (SEQ ID NO: 19 of US20160369298; herein SEQ ID NO: 2105), YYLSRTNTESGTPTQSALEFSQAGA (SEQ ID NO: 20 of US20160369298; herein SEQ ID NO: 2106), YYLSRTNTHSGTHTQSPLHFSQAGA (SEQ ID NO: 21 of US20160369298; herein SEQ ID NO: 2107), YYLSRTNTSSGTITISHLIFSQAGA (SEQ ID NO: 22 of US20160369298; herein SEQ ID NO: 2108), YYLSRTNTRSGIMTKSSLMFSQAGA (SEQ ID NO: 23 of US20160369298; herein SEQ ID NO: 2109), YYLSRTNTKSGRKTLSNLSFSQAGA (SEQ ID NO: 24 of US20160369298; herein SEQ ID NO: 2110), YYLSRTNDGSGPVTPSKLRFSQRGA (SEQ ID NO: 25 of US20160369298; herein SEQ ID NO: 2111), YYLSRTNAASGHATHSDLKFSQPGA (SEQ ID NO: 26 of US20160369298; herein SEQ ID NO: 2112), YYLSRINGQAGSLTMSELGFSQVGA (SEQ ID NO: 27 of US20160369298; herein SEQ ID NO: 2113), YYLSRINSTGGNQTTSQLLFSQLSA (SEQ ID NO: 28 of US20160369298; herein SEQ ID NO: 2114), YFLSRTNNNTGLNTNSTLNFSQGRA (SEQ ID NO: 29 of US20160369298; herein SEQ ID NO: 2115), SKTGADNNNSEYSWTG (SEQ ID NO: 30 of US20160369298; herein SEQ ID NO: 2116), SKTDADNNNSEYSWTG (SEQ ID NO: 31 of US20160369298; herein SEQ ID NO: 2117), SKTEADNNNSEYSWTG (SEQ ID NO: 32 of US20160369298; herein SEQ ID NO: 2118). SKTPADNNNSEYSWTG (SEQ ID NO: 33 of US20160369298; herein SEQ ID NO: 2119), SKTHADNNNSEYSWTG (SEQ ID NO: 34 of US20160369298; herein SEQ ID NO: 2120), SKTQADNNNSEYSWTG (SEQ ID NO: 35 of US20160369298; herein SEQ ID NO: 2121), SKTIADNNNSEYSWTG (SEQ ID NO: 36 of US20160369298; herein SEQ ID NO: 2122), SKTMADNNNSEYSWTG (SEQ ID NO: 37 of US20160369298; herein SEQ ID NO: 2123), SKTRADNNNSEYSWTG (SEQ ID NO: 38 of US20160369298; herein SEQ ID NO: 2124), SKTNADNNNSEYSWTG (SEQ ID NO: 39 of US20160369298; herein SEQ ID NO: 2125), SKTVGRNNNSEYSWTG (SEQ ID NO: 40 of US20160369298; herein SEQ ID NO: 2126), SKTADRNNNSEYSWTG (SEQ ID NO: 41 of US20160369298; herein SEQ ID NO: 2127), SKKLSQNNNSKYSWQG (SEQ ID NO: 42 of US20160369298; herein SEQ ID NO: 2128), SKPTTGNNNSDYSWPG (SEQ ID NO: 43 of US20160369298; herein SEQ ID NO: 2129), STQKNENNNSNYSWPG (SEQ ID NO: 44 of US20160369298; herein SEQ ID NO: 2130), HKDDEGKF (SEQ ID NO: 45 of US20160369298; herein SEQ ID NO: 2131), HKDDNRKF (SEQ NO: 46 of US20160369298; herein SEQ ID NO: 2132), HKDDTNKF (SEQ ID NO: 47 of US20160369298; herein SEQ ID NO: 2133), HEDSDKNF (SEQ ID NO: 48 of US20160369298; herein SEQ ID NO: 2134), HRDGADSF (SEQ ID NO: 49 of US20160369298; herein SEQ ID NO: 2135), HGDNKSRF (SEQ ID NO: 50 of US20160369298; herein SEQ ID NO: 2136). KQGSEKTNVDFEEV (SEQ ID NO: 51 of US20160369298; herein SEQ ID NO: 2137), KQGSEKTNVDSEEV (SEQ ID NO: 52 of US20160369298; herein SEQ ID NO: 2138). KQGSEKTNVDVEEV (SEQ ID NO: 53 of US20160369298; herein SEQ ID NO: 2139), KQGSDKTNVDDAGV (SEQ ID NO: 54 of US20160369298; herein SEQ ID NO: 2140), KQGSSKTNVDPREV (SEQ ID NO: 55 of US20160369298; herein SEQ ID NO: 2141), KQGSRKTNVDHKQV (SEQ ID NO: 56 of US20160369298; herein SEQ ID NO: 2142), KQGSKGGNVDTNRV (SEQ ID NO: 57 of US20160369298; herein SEQ ID NO: 2143), KQGSGEANVDNGDV (SEQ ID NO: 58 of US20160369298; herein SEQ ID NO: 2144), KQDAAADNIDYDHV (SEQ ID NO: 59 of US20160369298; herein SEQ ID NO: 2145). KQSGTRSNAAASSV (SEQ ID NO: 60 of US20160369298; herein SEQ ID NO: 2146), KENTNTNDTELTNV (SEQ ID NO: 61 of US20160369298; herein SEQ ID NO: 2147), QRGNNVAATADVNT (SEQ NO: 62 of US20160369298; herein SEQ ID NO: 2148), QRGNNEAATADVNT (SEQ ID NO: 63 of US20160369298; herein SEQ ID NO: 2149), QRGNNPAATADVNT (SEQ ID NO: 64 of US20160369298; herein SEQ ID NO: 2150), QRGNNHAATADVNT (SEQ ID NO: 65 of US20160369298; herein SEQ ID NO: 2151), QEENNIAATPGVNT (SEQ ID NO: 66 of US20160369298; herein SEQ ID NO: 2152). QPPNNMAATHEVNT (SEQ ID NO: 67 of US20160369298; herein SEQ ID NO: 2153), QHHNNSAATTIVNT (SEQ ID NO: 68 of US20160369298; herein SEQ ID NO: 2154), QTTNNRAAFNMVET (SEQ ID NO: 69 of US20160369298; herein SEQ ID NO: 2155), QKKNNNAASKKVAT (SEQ ID NO: 70 of US20160369298; herein SEQ ID NO: 2156), QGGNNKAADDAVKT (SEQ ID NO: 71 of US20160369298; herein SEQ ID NO: 2157), QAAKGGAADDANKT (SEQ ID NO: 72 of US20160369298; herein SEQ ID NO: 2158), QDDRAAAANESVDT (SEQ m NO: 73 of US20160369298; herein SEQ ID NO: 2159). QQQHDDAAYQRVHT (SEQ ID NO: 74 of US20160369298; herein SEQ ID NO: 2160), QSSSSLAAVSTVQT (SEQ ID NO: 75 of US20160369298; herein SEQ ID NO: 2161), QNNQTTAAIRNVTT (SEQ ID NO: 76 of US20160369298; herein SEQ ID NO: 2162), NYNKKSDNVDFT (SEQ ID NO: 77 of US20160369298; herein SEQ ID NO: 2163), NYNKKSENVDFT (SEQ ID NO: 78 of US20160369298; herein SEQ ID NO: 2164), NYNKKSLNVDFT (SEQ ID NO: 79 of US20160369298; herein SEQ ID NO: 2165), NYNKKSPNVDFT (SEQ ID NO: 80 of US20160369298; herein SEQ ID NO: 2166), NYSKKSHCVDFT (SEQ ID NO: 81 of US20160369298; herein SEQ ID NO: 2167), NYRKTIYVDFT (SEQ ID NO: 82 of US20160369298; herein SEQ ID NO: 2168), NYKEKKDVHFT (SEQ ID NO: 83 of US20160369298; herein SEQ ID NO: 2169), NYGHRAIVQFT (SEQ ID NO: 84 of US20160369298; herein SEQ ID NO: 2170), NYANHQFVVCT (SEQ ID NO: 85 of US20160369298; herein SEQ ID NO: 2171), NYDDDPTGVLLT (SEQ ID NO: 86 of US20160369298; herein SEQ ID NO: 2172), NYDDPTGAILLT (SEQ ID NO: 87 of US20160369298; herein SEQ ID NO: 2173), NFEQQNSVEWT (SEQ ID NO: 88 of US20160369298; herein SEQ ID NO: 2174), SQSGASN (SEQ ID NO: 89 and SEQ ID NO: 241 of US20160369298; herein SEQ ID NO: 2175), NNGSQA (SEQ ID NO: 90 of US20160369298; herein SEQ ID NO: 2176), YYLSRTNTPSGTTTWSRLQFSQAGA (SEQ ID NO: 91 of US20160369298; herein SEQ ID NO: 2177), SKTSADNNNSEYSWTG (SEQ ID NO: 92 of US20160369298; herein SEQ ID NO: 2178), HKDDEEKF (SEQ ID NO: 93, 209. 214, 219, 224, 234, 239, and 244 of US20160369298; herein SEQ ID NO: 2179), KQGSEKTNVDIEEV (SEQ ID NO: 94 of US20160369298; herein SEQ ID NO: 2180), QRGNNQAATADVNT (SEQ ID NO: 95 of US20160369298; herein SEQ ID NO: 2181), NYNKKSVNVDFT (SEQ ID NO: 96 of US20160369298; herein SEQ ID NO: 2182), SQSGASNYNTPSGTTTQSRLQFSTSADNNNSEYSSWTGATKYH (SEQ ID NO: 106 of US20160369298; herein SEQ ID NO: 2183), SASGASNFNSEGGSLTQSSLGFSTDGENNNSDFSWTGATKYH (SEQ ID NO: 107 of US20160369298; herein SEQ ID NO: 2184), SQSGASNYNTPSGTTTQSRLQFSTDGENNNSDFSWTGATKYH (SEQ ID NO: 108 of US20160369298; herein SEQ ID NO: 2185), SASGASNYNTPSGTTTQSRLQFSTSADNNNSEFSWPGATTYH (SEQ ID NO: 109 of US20160369298; herein SEQ ID NO: 2186), SQSGASNFNSEGGSLTQSSLGFSTDGENNNSDFSWTGATKYH (SEQ ID NO: 110 of US20160369298; herein SEQ ID NO: 2187), SASGASNYNTPSGSLTQSSLGFSTDGENNNSDFSWTGATKYH (SEQ ID NO: 111 of US 20160369298; herein SEQ ID NO: 2188), SQSGASNYNTPSGTTTQSRLQFSTSADNNNSDFSWTGATKYH (SEQ ID NO: 112 of US20160369298; herein SEQ ID NO: 2189), SGAGASNFNSEGGSLTQSSLGFSTDGENNNSDFSWTGATKYH (SEQ ID NO: 113 of US20160369298; herein SEQ ID NO: 2190), SGAGASN (SEQ ID NO: 176 of US20160369298; herein SEQ NO: 2191), NSEGGSLTQSSLGFS (SEQ ID NO: 177, 185. 193 and 202 of US20160369298; herein SEQ ID NO: 2192), TDGENNNSDFS (SEQ ID NO: 178 of US20160369298; herein SEQ ID NO: 2193), SEFSWPGATT (SEQ ID NO: 179 of US20160369298; herein SEQ ID NO: 2194), TSADNNNSDFSWT (SEQ ID NO: 180 of US20160369298; herein SEQ ID NO: 2195), SQSGASNY (SEQ ID NO: 181, 187, and 198 of US20160369298; herein SEQ ID NO: 2196), NTPSGTTTQSRLQFS (SEQ ID NO: 182, 188, 191, and 199 of US20160369298; herein SEQ ID NO: 2197), TSADNNNSEYSWTGATKYH (SEQ ID NO: 183 of US20160369298; herein SEQ ID NO: 2198), SASGASNF (SEQ ID NO: 184 of US20160369298; herein SEQ ID NO: 2199), TDGENNNSDFSWTGATKYH (SEQ NO: 186, 189, 194, 197, and 203 of US20160369298; herein SEQ ID NO: 2200), SASGASNY (SEQ ID NO: 190 and SEQ ID NO: 195 of US20160369298; herein SEQ ID NO: 2201). TSADNNNSEFSWPGATTYH (SEQ ID NO: 192 of US20160369298; herein SEQ ID NO: 2202), NTPSGSLTQSSLGFS (SEQ ID NO: 196 of US20160369298; herein SEQ ID NO: 2203), TSADNNNSDFSWTGATKYH (SEQ ID NO: 200 of US20160369298; herein SEQ ID NO: 2204), SGAGASNF (SEQ ID NO: 201 of US20160369298; herein SEQ ID NO: 2205), CTCCAGVVSVVSMRSRVCVNSGCAGCTDHCVVSRNSGTCVMSACACAA (SEQ ID NO: 204 of US20160369298; herein SEQ ID NO: 2206), CTCCAGAGAGGCAACAGACAAGCAGCTACCGCAGATGTCAACACACAA (SEQ ID NO: 205 of US20160369298; herein SEQ ID NO: 2207), SAAGASN (SEQ ID NO: 206 of US20160369298; herein SEQ ID NO: 2208), YFLSRTNTESGSTTQSTLRFSQAG (SEQ ID NO: 207 of US20160369298; herein SEQ ID NO: 2209), SKTSADNNNSDFS (SEQ ID NO: 208, 228, and 253 of US20160369298; herein SEQ ID NO: 2210), KQGSEKTDVDIDKV (SEQ ID NO: 210 of US20160369298; herein SEQ ID NO: 2211), STAGASN (SEQ ID NO: 211 of US20160369298; herein SEQ ID NO: 2212), YFLSRTNTTSGIETQSTLRFSQAG (SEQ ID NO: 212 and SEQ ID NO: 247 of 0520160369298; herein SEQ ID NO: 2213), SKTDGENNNSDFS (SEQ ID NO: 213 and SEQ ID NO: 248 of US20160369298; herein SEQ ID NO: 2214), KQGAAADDVEIDGV (SEQ ID NO: 215 and SEQ ID NO: 250 of US20160369298; herein SEQ ID NO: 2215), SEAGASN (SEQ ID NO: 216 of US20160369298; herein SEQ ID NO: 2216), YYLSRTNTPSGTTTQSRLQFSQAG (SEQ ID NO: 217, 232 and 242 of US20160369298; herein SEQ ID NO: 2217), SKTSADNNNSEYS (SEQ ID NO: 218, 233, 238, and 243 of US20160369298; herein SEQ ID NO: 2218). KQGSEKTNVDIEKV (SEQ ID NO: 220, 225 and 245 of US20160369298; herein SEQ ID NO: 2219), YFLSRTNDASGSDTKSTLLFSQAG (SEQ ID NO: 222 of US20160369298; herein SEQ ID NO: 2220), STTPSENNNSEYS (SEQ ID NO: 223 of US20160369298; herein SEQ ID NO: 2221), SAAGATN (SEQ ID NO: 226 and SEQ ID NO: 251 of US20160369298; herein SEQ ID NO: 2222), YFLSRTNGEAGSATLSELRFSQAG (SEQ ID NO: 227 of US20160369:298; herein SEQ ID NO: 2223), HGDDADRF (SEQ ID NO: 229 and SEQ ID NO: 254 of US20160369298; herein SEQ ID NO: 2224), KQGAEKSDVEVDRV (SEQ ID NO: 230 and SEQ ID NO: 255 of US20160369298; herein SEQ ID NO: 2225), KQDSGGDNIDIDQV (SEQ ID NO: 235 of US20160369298; herein SEQ ID NO: 2226), SDAGASN (SEQ ID NO: 236 of US20160369298; herein SEQ ID NO: 2227), YFLSRTNTEGGHDTQSTLRFSQAG (SEQ ID NO: 237 of US20160369298; herein SEQ ID NO: 2228), KEDGGGSDVAIDEV (SEQ ID NO: 240 of US20160369298; herein SEQ ID NO: 2229), SNAGASN (SEQ ID NO: 246 of US20160369298; herein SEQ ID NO: 2230), and YFLSRTNGEAGSATLSELRFSQPG (SEQ ID NO: 252 of US20160369298; herein SEQ ID NO: 2231). Non-limiting examples of nucleotide sequences that may encode the amino acid mutated sites include the following, AGCVVMDCAGGARSCASCAAC (SEQ ID NO: 97 of US20160369298; herein SEQ ID NO: 2232), AACRACRRSMRSMAGGCA (SEQ ID NO: 98 of US20160369298; herein SEQ NO: 2233), CACRRGGACRRCRMSRRSARSTTT (SEQ ID NO: 99 of US20160369298; herein SEQ ID NO: 2234), TATTTCTTGAGCAGAACAAACRVCVVSRSCGGAMNCVHSACGMHSTCAVVSCTTVDS TTTTCTCAGSBCRGSGCG (SEQ ID NO: 100 of US20160369298, herein SEQ ID NO: 2235), TCAAMAMMAVNSRVCSRSAACAACAACAGTRASTTCTCGTGGMMAGGA (SEQ ID NO: 101 of US20160369298; herein SEQ ID NO: 2236), AAGSAARRCRSCRVSRVARVCRATRYCGMSNHCRVMVRSGTC (SEQ ID NO: 102 of US20160369298; herein SEQ ID NO: 2237), CAGVVSVVSMRSRVCVNSGCAGCTDHCVVSRNSGTCVMSACA (SEQ ID NO: 103 of US20160369298; herein SEQ ID NO: 2238), AACTWCRVSVASMVSVHSDDTGTGSWSTKSACT (SEQ ID NO: 104 of US20160369298; herein SEQ ID NO: 2239), TTGTTGAACATCACCACGTGACGCACGTTC (SEQ ID NO: 256 of US20160369298; herein SEQ ID NO: 2240). TCCCCGTGGTTCTACTACATAATGTGGCCG (SEQ ID NO: 257 of US20160369298; herein SEQ ID NO: 2241), TTCCACACTCCGTTTGGATAATGTTGAAC (SEQ ID NO: 258 of US20160369298; herein SEQ ID NO: 2242), AGGGACATCCCCAGCTCCATGCTGTGGTCG (SEQ ID NO: 259 of US20160369298; herein SEQ ID NO: 2243), AGGGACAACCCCTCCGACTCGCCCTAATCC (SEQ ID NO: 260 of US20160369298; herein SEQ ID NO: 2244), TCCTAGTAGAAGACACCCTCTCACTGCCCG (SEQ ID NO: 261 of US20160369298; herein SEQ ID NO: 2245), AGTACCATGTACACCCACTCTCCCAGTGCC (SEQ ID NO: 262 of US20160369298; herein SEQ ID NO: 2246), ATATGGACGTTCATGCTGATCACCATACCG (SEQ ID NO: 263 of US20160369298; herein SEQ ID NO: 2247), AGCAGGAGCTCCTTGGCCTCAGCGTGCGAG (SEQ ID NO: 264 of US20160369298; herein SEQ ID NO: 2248), ACAAGCAGCTTCACTATGACAACCACTGAC (SEQ ID NO: 265 of US20160369298; herein SEQ ID NO: 2249), CAGCCTAGGAACTGGCTTCCTGGACCCTGTTACCGCCAGCAGAGAGTCTCAAMAMM AVNSRVCSRSAACAACAACAGTRASTTCTCCTGGMMAGGAGCTACCAAGTACCACC TCAATGGCAGAGACTCTCTGGTGAATCCCGGACCAGCTATGGCAAGCCACRRGGAC RRCRMSRRSARSTTTTTTCCTCAGAGCGGGGTTCTCATCTTTGGGAAGSAARRCRSCR VSRVARVCRATRYCGMSNHCRVMVRSGTCATGATTACAGACGAAGAGGAGATCTGG AC (SEQ ID NO: 266 of US20160369298; herein SEQ ID NO: 2250), TGGGACAATGGCGGTCGTCTCTCAGAGTTKTKKT (SEQ ID NO: 267 of US20160369298; herein SEQ ID NO: 2251), AGAGGACCKKTCCTCGATGGTTCATGGTGGAGTTA (SEQ ID NO: 268 of US20160369298; herein SEQ ID NO: 2252), CCACTTAGGGCCTGGTCGATACCGTTCGGTG (SEQ ID NO: 269 of US20160369298; herein SEQ ID NO: 2253), and TCTCGCCCCAAGAGTAGAAACCCTTCSTTYYG (SEQ ID NO: 270 of US20160369298; herein SEQ ID NO: 2254).

In some embodiments, the AAV serotype may comprise an ocular cell targeting peptide as described in International Patent Publication WO2016134375, the contents of which are herein incorporated by reference in their entirety, such as, but not limited to SEQ ID NO: 9, and SEQ ID NO:10 of WO2016134375. Further, any of the ocular cell targeting peptides or amino acids described in WO2016134375, may be inserted into any parent AAV serotype, such as, but not limited to, AAV2 (SEQ ID NO:8 of WO2016134375; herein SEQ ID NO: 2255), or AAV9 (SEQ ID NO: 11 of WO2016134375; herein SEQ ID NO: 2256). In some embodiments, modifications, such as insertions are made in AAV2 proteins at P34-A35, T138-A139, A139-P140, G453-T454, N587-R588, and/or R588-Q589. In certain embodiments, insertions are made at D384, G385, 1560, T561, N562, E563, E564, E565, N704, and/or Y705 of AAV9. The ocular cell targeting peptide may be, but is not limited to, any of the following amino acid sequences, GSTPPPM (SEQ ID NO: 1 of 402016134375; herein SEQ ID NO: 2257), or GETRAPL (SEQ ID NO: 4 of WO2016134375; herein SEQ ID NO: 2258).

In some embodiments, the AAV serotype may be modified as described in the United States Publication US 20170145405 the contents of which are herein incorporated by reference in their entirety. AAV serotypes may include, modified AAV2(e.g., modifications at Y444F, Y500F, Y730F and/or S662V), modified AAV3 (e.g., modifications at Y705F, Y731F and/or T492V), and modified AAV6 (e.g., modifications at S663V and/or T492V),

In some embodiments, the AAV serotype may be modified as described in the International Publication WO2017083722 the contents of which are herein incorporated by reference in their entirety. AAV serotypes may include, AAV1 (Y705+731F+T492V), AAV2 (Y444+500+730F+T491V), AAV3 (Y705+731F), AAV5, AAV 5(Y436+693+719F), AAV6 (VP3 variant Y705F/Y731F/T492V), AAV8 (Y733F), AAV9, AAV9 (VP3 variant Y731 F), and AAV10 (Y733F).

In some embodiments, the AAV serotype may comprise, as described in International Patent Publication WO2017015102, the contents of which are herein incorporated by reference in their entirety, an engineered epitope comprising the amino acids SPAKFA (SEQ ID NO: 24 of WO2017015102; herein SEQ ID NO: 2259) or NKDKLN (SEQ NO:2 of WO2017015102; herein SEQ ID NO: 2260). The epitope may be inserted in the region of amino acids 665 to 670 based on the numbering of the VPI capsid of AAV8 (SEQ ID NO:3 of WO2017015102) and/or residues 664 to 668 of AAV3B (SEQ ID NO:3).

In some embodiments, the AAV serotype may be, or may have a sequence as described in International Patent Publication WO2017058892, the contents of which are herein incorporated by reference in their entirety, such as, but not limited to, AAV variants with capsid proteins that may comprise a substitution at one or more (e.g., 2, 3, 4, 5, 6, or 7) of amino acid residues 262-268, 370-379, 451-459, 472-473, 493-500, 528-534, 547-552, 588-597, 709-710, 716-722 of AAV1, in any combination, or the equivalent amino acid residues in AAV2, AAV3, AAV4, AAVS, AAV6, AAV7, AAV8, AAV9, AAV10, AAV 11, AAV12, AAVrh8, AAVrh10, AAVrh32.33, bovine AAV or avian AAV. The amino acid substitution may be, but is not limited to, any of the amino acid sequences described in WO2017058892. In some embodiments, the AAV may comprise an amino acid substitution at residues 256L, 258K, 259Q, 261S, 263A, 264S, 265T, 266G, 272H, 385S, 386Q, S472R, V473D, N500E 547S, 709A, 710N, 716D, 717N, 718N, 720L, A456T, Q457T, N458Q, K459S, T492S, K493A, S586R, S587G, S588N, T589R, and/or 722T of AAV1 (SEQ ID NO: 1 of WO2017058892) in any combination, 244N, 246Q, 248R, 249E, 2501, 251K, 252S, 253G, 254S, 255V, 256D, 263Y, 377E, 378N, 453L, 456R, 532Q, 533P, 535N, 536P, 537G, 538T, 539T, 540A, 541T, 542Y, 543L, 546N, 653V, 654P, 656S, 697Q, 698F, 704D, 705S, 706T, 707G, 708E, 709Y and/or 710R of AAV5 (SEQ ID NO:5 of WO2017058892) in any combination, 248R, 316V, 317Q, 318D, 319S, 443N, 530N, 531S, 532Q 533P, 534A, 535N, 540A, 541 T, 542Y, 543L, 545G, 546N, 697Q, 704D, 706T, 708E, 709Y and/or 710R of AAV5 (SEQ ID NO: 5 of WO2017058892) in any combination, 264S, 266G, 269N, 272H, 457Q, 588S and/or 589I of AAV6 (SEQ ID NO:6 WO2017058892) in any combination, 457T, 459N, 496G, 499N, 500N, 589Q, 590N and/or 592A of AAV8 (SEQ ID NO: 8 WO2017058892) in any combination,451I, 452N, 453G, 454S, 455G, 456Q, 457N and/or 458Q of AAV9 (SEQ ID NO: 9 WO2017058892) in any combination.

In some embodiments, the AAV may include a sequence of amino acids at positions 155, 156 and 157 of VP1 or at positions 17, 18, 19 and 20 of VP2, as described in International Publication No. WO 2017066764 the contents of which are herein incorporated by reference in their entirety. The sequences of amino acid may be, but not limited to, N-S-S, S-X-S, S-S-Y, N-X-S, N-S-Y, S-X-Y and N-X-Y, where N, X and Y are, but not limited to, independently non-serine, or non-threonine amino acids, wherein the AAV may be, but not limited to AAV1, AAV2, AAV3, AAV4, AAV5, AAV6, AAV7, AAV8, AAV9, AAV10, AAV11 and AAV12. In some embodiments, the AAV may include a deletion of at least one amino acid at positions 156, 157 or 158 of VP1 or at positions 19, 20 or 21 of VP2, wherein the AAV may be, but not limited to AAV1, AAV2, AAV3, AAV4, AAV5, AAV6, AAV7, AAV8, AAV9, AAV10, AAV11 and AAV12.

In some embodiments, the AAV serotype may be as described in Jackson et al (Frontiers in Molecular Neuroscience 9:154 (2016)), the contents of which are herein incorporated by reference in their entirety. In some embodiments, the AAV serotype is PHP.B or AAV9. In some embodiments, the AAV serotype is paired with a synapsin promoter to enhance neuronal transduction, as compared to when more ubiquitous promoters are used (i.e., CBA or CMV).

In some embodiments, peptides for inclusion in an AAV serotype may be identified by isolating human splenocytes, re-stimulating the splenocytes in vitro using individual peptides spanning the amino acid sequence of the AAV capsid protein, IFN-gamma ELISpot with the individual peptides used for the in vitro re-stimulation, bioinformatics analysis to determine the given allele restriction of 15-mers identified by IFN-gamma ELISpot, identification of candidate reactive 9-mer epitopes for a given allele, synthesis candidate 9-mers, second IFN-gamma ELISpot screening of splenocytes from subjects carrying the specific alleles to which identified AAV epitopes are predicted to bind, determine the AAV capsid-reactive CD8+ T-cell epitopes and determine the frequency of subjects reacting to a given AAV epitope,

AAV particles comprising a modulatory polynucleotide encoding the siRNA molecules may be prepared or derived from various serotypes of AAVs, including, but not limited to, AAV1, AAV2, AAV3, AAV4, AAVS, AAV6, AAV7, AAV8, AAV9, AAV9.47, AAV9(hu14), AAV10, AAV11, AAV12, AAVrh8, AAVrh10, AAV-DJ8 and AAV-DJ. In some cases, different serotypes of AAVs may be mixed together or with other types of viruses to produce chimeric AAV particles. As a non-limiting example, the AAV particle is derived from the AAV9 serotype.

### Viral Genome

In some embodiments, as shown in an AAV particle comprises a viral genome with a payload region.

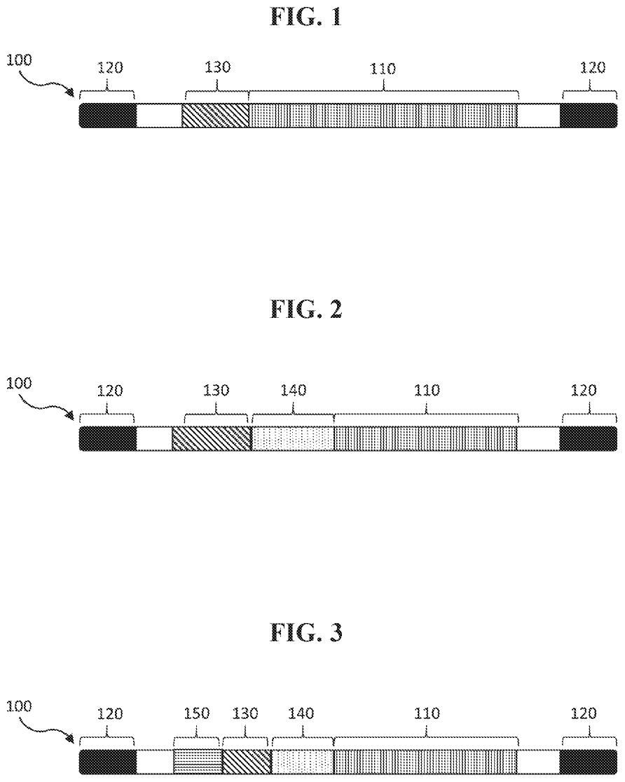

In some embodiments, the viral genome may comprise the components as shown in FIG. 1. The payload region 110 is located within the viral genome 100. At the 5′ and/or the 3′ end of the viral genome 100 there may be at least one inverted terminal repeat (ITR) 120. Between the 5′ ITR 120 and the payload region 110, there may be a promoter region 130. In some embodiments, the payload region may comprise at least one modulatory polynucleotide.

In some embodiments, the viral genome 100 may comprise the components as shown in FIG. 2. The payload region 110 is located within the viral genome 100. At the 5′ and/or the 3′ end of the viral genome 100 there may be at least one inverted terminal repeat (ITR) 120. Between the 5′ ITR 120 and the payload region 110, there may be a promoter region 130. Between the promoter region 130 and the payload region 110, there may be an intron region 140. In some embodiments, the payload region may comprise at least one modulatory polynucleotide.

In some embodiments, the viral genome 100 may comprise the components as shown in FIG. 3. At the 5′ and/or the 3′ end of the viral genome 100 there may be at least one inverted terminal repeat (ITR) 120. Within the viral genome 100, there may be an enhancer region 150, a promoter region 130, an intron region 140, and a payload region 110. In some embodiments, the payload region may comprise at least one modulatory polynucleotide.

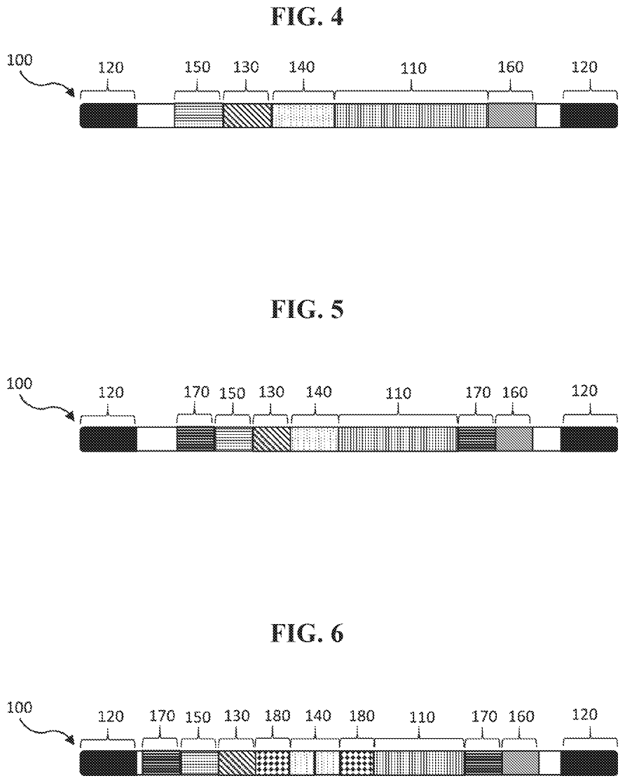

In some embodiments, the viral genome 100 may comprise the components as shown in FIG. 4. At the 5′ and/or the 3′ end of the viral genome 100 there may be at least one inverted terminal repeat (ITR) 120. Within the viral genome 100, there may be an enhancer region 150, a promoter region 130, an intron region 140, a payload region 110, and a polyadenylation signal sequence region 160. In some embodiments, the payload region may comprise at least one modulatory polynucleotide.

In some embodiments, the viral genome 100 may comprise the components as shown in FIG. 5. At the 5′ and/or the 3′ end of the viral genome 100 there may be at least one inverted terminal repeat (ITR) 120. Within the viral genome 100, there may be at least one MCS region 170, an enhancer region 150, a promoter region 130, an intron region 140, a payload region 110, and a polyadenylation signal sequence region 160. In some embodiments, the payload region may comprise at least one modulatory polynucleotide.

In some embodiments, the viral genome 100 may comprise the components as shown in FIG. 6. At the 5′ and/or the 3′ end of the viral genome 100 there may be at least one inverted terminal repeat (ITR) 120. Within the viral genome 100, there may be at least one MCS region 170, an enhancer region 150, a promoter region 130, at least one exon region 180, at least one intron region 140, a payload region 110, and a polyadenylation signal sequence region 160. In some embodiments, the payload region may comprise at least one modulatory polynucleotide.

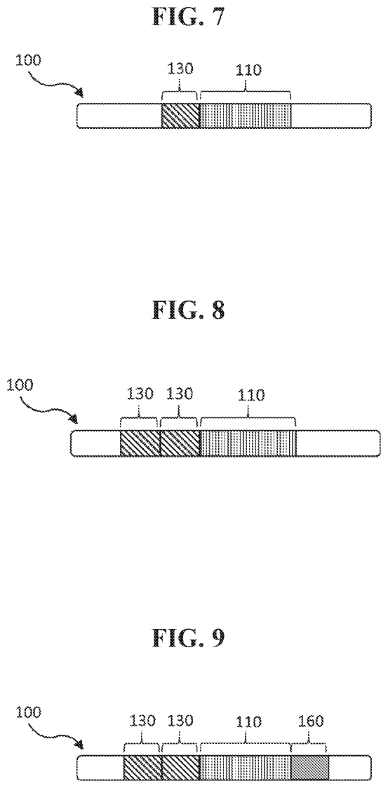

In some embodiments, the viral genome 100 may comprise the components as shown in FIGS. 7 and 8. Within the viral genome 100, there may be at least one promoter region 130, and a payload region 110. In some embodiments, the payload region may comprise at least one modulatory polynucleotide.

In some embodiments, the viral genome 100 may comprise the components as shown in FIG. 9. Within the viral genome 100, there may be at least one promoter region 130, a payload region 110, and a polyadenylation signal sequence region 160. In some embodiments, the payload region may comprise at least one modulatory polynucleotide.

### Viral Genome Size

In some embodiments, the viral genome which comprises a payload described herein, may be single stranded or double stranded viral genome. The size of the viral genome may be small, medium, large or the maximum size. Additionally, the viral genome may comprise a promoter and a polyA tail.

In some embodiments, the viral genome which comprises a payload described herein, may be a small single stranded viral genome. A small single stranded viral genome may be 2.7 to 3.5 kb in size such as about 2.7, 2.8, 2.9, 3.0, 3.1, 3.2, 3.3, 3.4, and 3.5 kb in size. As a non-limiting example, the small single stranded viral genome may be 3.2 kb in size. Additionally, the viral genome may comprise a promoter and a polyA tail.

In some embodiments, the viral genome which comprises a payload described herein, may be a small double stranded viral genome. A small double stranded viral genome may be 1.3 to 1.7 kb in size such as about 1.3, 1.4, 1.5, 1.6, and 1.7 kb in size. As a non-limiting example, the small double stranded viral genome may be 1.6 kb in size. Additionally, the viral genome may comprise a promoter and a polyA tail.

In some embodiments, the viral genome which comprises a payload described herein, may a medium single stranded viral genome. A medium single stranded viral genome may be 3.6 to 4.3 kb in size such as about 3.6, 3.7, 3.8, 3.9, 4.0, 4.1, 4.2 and 4.3 kb in size. As a non-limiting example, the medium single stranded viral genome may be 4.0 kb in size. Additionally, the viral genome may comprise a promoter and a polyA tail.

In some embodiments, the viral genome which comprises a payload described herein, may be a medium double stranded viral genome. A medium double stranded viral genome may be 1.8 to 2.1 kb in size such as about 1.8, 1.9, 2.0, and 2.1 kb in size. As a non-limiting example, the medium double stranded viral genome may be 2.0 kb in size. Additionally, the viral genome may comprise a promoter and a polyA tail.

In some embodiments, the viral genome which comprises a payload described herein, may be a large single stranded viral genome. A large single stranded viral genome may be 4.4 to 6.0 kb in size such as about 4.4, 4.5, 4.6, 4.7, 4.8, 4.9, 5.0, 5.1, 5.2, 5.3, 5.4, 5.5, 5.6, 5.7, 5.8, 5.9 and 6.0 kb in size. As a non-limiting example, the large single stranded viral genome may be 4.7 kb in size. As another non-limiting example, the large single stranded viral genome may be 4.8 kb in size. As yet another non-limiting example, the large single stranded viral genome may be 6.0 kb in size. Additionally, the viral genome may comprise a promoter and a polyA tail.

In some embodiments, the viral genome which comprises a payload described herein, may be a large double stranded viral genome. A large double stranded viral genome may be 2.2 to 3.0 kb in size such as about 2.2, 2.3, 2.4, 2.5, 2.6, 2.7, 2.8, 2.9 and 3.0 kb in size. As a non-limiting example, the large double stranded viral genome may be 2.4 kb in size. Additionally, the viral genome may comprise a promoter and a polyA tail.

### Viral Genome Component: Inverted Terminal Repeats (ITRs)

101701 The AAV particles of the present disclosure comprise a viral genome with at least one ITR region and a payload region. In some embodiments the viral genome has two ITRs. These two ITRs flank the payload region at the 5′ and 3′ ends. The ITRs function as origins of replication comprising recognition sites for replication. ITRs comprise sequence regions which can be complementary and symmetrically arranged. ITRs incorporated into viral genomes of the disclosure may be comprised of naturally occurring polynucleotide sequences or recombinantly derived polynucleotide sequences.

The ITRs may be derived from the same serotype as the capsid, selected from any of the serotypes listed in Table 1, or a derivative thereof. The ITR may be of a different serotype from the capsid. In some embodiments the AAV particle has more than one ITR. In a non-limiting example, the AAV particle has a viral genome comprising two ITRs. In some embodiments the ITRs are of the same serotype as one another. In another embodiment the ITRs are of different serotypes. Non-limiting examples include zero, one or both of the ITRs having the same serotype as the capsid. In some embodiments both ITRs of the viral genome of the AAV particle are AAV2 ITRs.

Independently, each ITR may be about 100 to about 150 nucleotides in length. An ITR may be about 100-105 nucleotides in length, 106-110 nucleotides in length, 111-115 nucleotides in length, 116-120 nucleotides in length, 121-125 nucleotides in length, 126-130 nucleotides in length, 131-135 nucleotides in length, 136-140 nucleotides in length, 141-145 nucleotides in length or 146-150 nucleotides in length. In some embodiments the ITRs are 140-142 nucleotides in length. Non limiting examples of ITR length are 102, 140, 141, 142, 145 nucleotides in length, and those having at least 95% identity thereto.

In some embodiments, the AAV particle comprises a nucleic acid sequence encoding an siRNA molecule which may be located near the 5′ end of the flip ITR in an expression vector. In another embodiment, the AAV particle comprises a nucleic acid sequence encoding an siRNA molecule may be located near the 3′ end of the flip ITR in an expression vector. In yet another embodiment, the AAV particle comprises a nucleic acid sequence encoding an siRNA molecule may be located near the 5′ end of the flop ITR in an expression vector. In yet another embodiment, the AAV particle comprises a nucleic acid sequence encoding an siRNA molecule may be located near the 3′ end of the flop ITR in an expression vector. In some embodiments, the AAV particle comprises a nucleic acid sequence encoding an siRNA molecule may be located between the 5′ end of the flip ITR and the 3′ end of the flop ITR in an expression vector. In some embodiments. the AAV particle comprises a nucleic acid sequence encoding an siRNA molecule may be located between (e.g., half-way between the 5′ end of the flip ITR and 3′ end of the flop ITR or the 3′ end of the flop ITR and the 5′ end of the flip ITR), the 3′ end of the flip ITR and the 5′ end of the flip ITR in an expression vector. As a non-limiting example, the AAV particle comprises a nucleic acid sequence encoding an siRNA molecule may be located within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more than 30 nucleotides downstream from the 5′ or 3′ end of an ITR (e.g., Flip or Flop ITR) in an expression vector. As a non-limiting example. the AAV particle comprises a nucleic acid sequence encoding an siRNA molecule may be located within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more than 30 nucleotides upstream from the 5′ or 3′ end of an ITR (e.g., Flip or Flop ITR) in an expression vector. As another non-limiting example, the AAV particle comprises a nucleic acid sequence encoding an siRNA molecule may be located within 1-5, 1-10, 1-15, 1-20, 1-25, 1-30, 5-10, 5-15, 5-20, 5-25, 5-30, 10-15, 10-20, 10-25, 10-30, 15-20, 15-25, 15-30, 20-25, 20-30 or 25-30 nucleotides downstream from the 5′ or 3′ end of an ITR (e.g., Flip or Flop ITR) in an expression vector. As another non-limiting example, the AAV particle comprises a nucleic acid sequence encoding an siRNA molecule may be located within 1-5, 1-10. 1-15, 1-20, 1-25, 1-30, 5-10, 5-15, 5-20, 5-25. 5-30, 10-15, 10-20, 10-25, 10-30, 15-20, 15-25, 15-30, 20-25, 20-30 or 25-30 upstream from the 5′ or 3′ end of an ITR (e.g., Flip or Flop ITR) in an expression vector. As a non-limiting example, the AAV particle comprises a nucleic acid sequence encoding an siRNA molecule may be located within the first 1%, 2%, 3%, 4%, 5%, 6%, 7%, 8%, 9%, 10%, 15%, 20%, 25% or more than 25% of the nucleotides upstream from the 5′ or 3′ end of an FIR (e.g., Flip or Flop ITR) in an expression vector. As another non-limiting example, the AAV particle comprises a nucleic acid sequence encoding an siRNA molecule may be located with the first 1-5%, 1-10%, 1-15%, 1-20%, 1-25%, 5-10%, 5-15%, 5-20%, 5-25%, 10-15%, 10-20%, 10-25%, 15-20%, 15-25%, or 20-25% downstream from the 5′ or 3′ end of an ITR (e.g., Flip or Flop ITR)) in an expression vector.

### Viral Genome Component: Promoters

In some embodiments, the payload region of the viral genome comprises at least one element to enhance the transgene target specificity and expression (See e.g., Powell et al. Viral Expression Cassette Elements to Enhance Transgene Target Specificity and Expression in Gene Therapy, 2015; the contents of which are herein incorporated by reference in its entirety). Non-limiting examples of elements to enhance the transgene target specificity and expression include promoters, endogenous miRNAs, post-transcriptional regulatory elements (PREs), polyadenylation (PolyA) signal sequences and upstream enhancers (USEs), CMV enhancers and introns.

A person skilled in the a may recognize that expression of the polypeptides of the disclosure in a target cell may require a specific promoter, including but not limited to, a promoter that is species specific, inducible, tissue-specific, or cell cycle-specific (Parr et al., Nat Med.3:1145-9 (1997); the contents of which are herein incorporated by reference in their entirety).

In some embodiments, the promoter is deemed to be efficient when it drives expression of the polypeptide(s) encoded in the payload region of the viral genome of the AAV particle.

In some embodiments, the promoter is a promoter deemed to be efficient to drive the expression of the modulatory polynucleotide.

In some embodiments, the promoter is a promoter deemed to be efficient when it drives expression in the cell being targeted.

In some embodiments, the promoter drives expression of the payload for a period of time in targeted tissues. Expression driven by a promoter may be for a period of 1 hour, 2, hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 1 week, 8 days, 9 days, 10 days, 11 days, 12 days, 13 days, 2 weeks, 15 days, 16 days, 17 days, 18 days, 19 days, 20 days, 3 weeks, 22 days, 23 days, 24 days, 25 days, 26 days, 27 days, 28 days, 29 days, 30 days, 31 days, 1 month, 2 months, 3 months, 4 months, 5 months, 6 months, 7 months, 8 months, 9 months, 10 months, 11 months, 1 year, 13 months, 14 months, 15 months, 16 months, 17 months, 18 months, 19 months, 20 months, 21 months, 22 months, 23 months, 2 years, 3 years, 4 years, 5 years, 6 years, 7 years, 8 years, 9 years, 10 years or more than 10 years. Expression may be for 1-5 hours, 1-12 hours, 1-2 days, 1-5 days, 1-2 weeks, 1-3 weeks, 1-4 weeks, 1-2 months, 1-4 months, 1-6 months, 2-6 months. 3-6 months, 3-9 months, 4-8 months, 6-12 months, 1-2 years, 1-5 years, 2-5 years, 3-6 years, 3-8 years, 4-8 years or 5-10 years.

In some embodiments, the promoter drives expression of the payload for at least 1 month, 2 months, 3 months, 4 months, 5 months, 6 months, 7 months, 8 months, 9 months, 10 months, 11 months, 1 year, 2 years, 3 years 4 years, 5 years, 6 years, 7 years, 8 years, 9 years, 10 years, 11 years, 12 years, 13 years, 14 years, 15 years, 16 years, 17 years, 18 years, 19 years, 20 years, 21 years, 22 years, 23 years, 24 years, 25 years, 26 years, 27 years, 28 years, 29 years, 30 years, 31 years, 32 years, 33 years, 34 years, 35 years, 36 years, 37 years, 38 years, 39 years, 40 years, 41 years, 42 years, 43 years, 44 years, 45 years, 46 years. 47 years, 48 ears, 49 years, 50 years, 55 years, 60 years, 65 years, or more than 65 years.

Promoters may be naturally occurring or non-naturally occurring. examples of promoters include viral promoters, plant promoters and mammalian promoters. In some embodiments, the promoters may be human promoters. In some embodiments, the promoter may be truncated.

Promoters which drive or promote expression in most tissues include, but are not limited to, human elongation factor 1α-subunit (EF1α) cytomegalovirus (CMV) immediate-early enhancer and/or promoter, chicken β-actin (CBA) and its derivative CAG, βglucuronidase (GUSB), or ubiquitin C (UBC). Tissue-specific expression elements can be used to restrict expression to certain cell types such as, but not limited to, muscle specific promoters, B cell promoters, monocyte promoters, leukocyte promoters, macrophage promoters, pancreatic acinar cell promoters, endothelial cell promoters, lung tissue promoters, astrocyte promoters, or nervous system promoters which can be used to restrict expression to neurons, astrocytes, or oligodendrocytes.

Non-limiting examples of muscle-specific promoters include mammalian muscle creatine kinase (NICK) promoter, mammalian desmin (DES) promoter, mammalian troponin I (TNNI2) promoter, and mammalian skeletal alpha-actin (ASK, promoter (see, e.g. U.S. Patent Publication US 20110212529, the contents of which are herein incorporated by reference in their entirety).

Non-limiting examples of tissue-specific expression elements for neurons include neuron-specific enolase (NSE), platelet-derived growth factor (PDGF), platelet-derived growth factor B-chain (PDGF-β), synapsin (Syn), methyl-CpG binding protein 2 (MeCP2), Ca²⁺/calmodulin-dependent protein kinase II (CaMKII), metabotropic glutamate receptor 2 neurofilament light (NFL) or heavy (NFH), β-globin minigene nβ2, preproenkephalin (PPE), enkephalin (Enk) and excitatory amino acid transporter 2 (EAAT2) promoters. Non-limiting examples of tissue-specific expression elements for astrocytes include glial fibrillary acidic protein (GFAP) and EAAT2 promoters. A non-limiting example of a tissue-specific expression element for oligodendrocytes includes the myelin basic protein (MBP) promoter.

In some embodiments, the promoter may be less than 1 kb. The promoter may have a length of 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800 or more than 800 nucleotides. The promoter may have a length between 200-300, 200-400, 200-500, 200-600, 200-700, 200-800, 300-400, 300-500, 300-600, 300-700, 300-800, 400-500, 400-600, 400-700, 400-800, 500-600, 500-700, 500-800, 600-700, 600-800 or 700-800.

In some embodiments, the promoter may be a combination of two or more components of the same or different starting or parental promoters such as, but not limited to, CMV and CBA. Each component may have a length of 200, 210, 220, 230, 240, 250, 260, 270, 280, 290, 300. 310, 320, 330, 340, 350, 360, 370. 380, 381, 382, 383, 384, 385, 386, 387, 388, 389, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800 or more than 800. Each component may have a length between 200-300, 200-400, 200-500, 200-600, 200-700, 200-800, 300-400, 300-500, 300-600, 300-700, 300-800, 400-500, 400-600, 400-700, 400-800, 500-600, 500-700, 500-800, 600-700, 600-800 or 700-800. In some embodiments, the promoter is a combination of a 382 nucleotide CMV-enhancer sequence and a 260 nucleotide CBA-promoter sequence.

In some embodiments, the viral genome comprises a ubiquitous promoter. Non-limiting examples of ubiquitous promoters include CMV, CBA (including derivatives CAG, CBh, etc.), EF-1α, PGK, UBC, GUSB (hGBp), and UCOE (promoter of HNRPA2B1-CBX3).

Yu et al, (Molecular Pain 2011, 7:63; the contents of which are herein incorporated by reference in their entirety) evaluated the expression of eGFP under the CAG, EFIα, PGK and UBC promoters in rat DRG cells and primary DRG cells using lentiviral vectors and found that UBC showed weaker expression than the other 3 promoters and only 10-12% glial expression was seen for all promoters. Soderblom et al. (E. Neuro 2015; the contents of which are herein incorporated by reference in its entirety) evaluated the expression of eGFP in AAV8 with CMV and UBC promoters and AAV2 with the CMV promoter after injection in the motor cortex. Intranasal administration of a plasmid containing a UBC or EF1α promoter showed a sustained airway expression greater than the expression with the CMV promoter (See e.g., Gill et al., Gene Therapy 2001, Vol. 8, 1539-1546; the contents of which are herein incorporated by reference in their entirety). Husain et al. (Gene Therapy 2009; the contents of which are herein incorporated by reference in its entirety) evaluated an HβH construct with a hGUSB promoter, a HSV-1LAT promoter and an NSE promoter and found that the HβH construct showed weaker expression than NSE in mouse brain. Passini and Wolfe (J. Virol. 2001, 12382-12392, the contents of which are herein incorporated by reference in its entirety) evaluated the long-term effects of the HβH vector following an intraventricular injection in neonatal mice and found that there was sustained expression for at least 1 year, Low expression in all brain regions was found by Xu et al. (Gene Therapy 2001, 8, 1323-1332; the contents of which are herein incorporated by reference in their entirety) when NFL and NFH promoters were used as compared to the CMV-lacZ, CMV-luc, EF, GFAP, hENK, nAChR, PPE, PPE+wpre, NSE (0.3 kb), NSE (1.8 kb) and NSE (1.8 kb+wpre). Xu et al. found that the promoter activity in descending order was NSE (1.8 kb), EF, NSE (0.3 kb), GFAP, CMV, hENK, PPE, NFL and NFH. NFL is a 650-nucleotide promoter and NFH is a 920-nucleotide promoter which are both absent in the liver but NFH is abundant in the sensory proprioceptive neurons, brain and spinal cord and NFH is present in the heart. Scn8a is a 470 nucleotide promoter which expresses throughout the DRG, spinal cord and brain with particularly high expression seen in the hippocampal neurons and cerebellar Purkinje cells, cortex, thalamus and hypothalamus (See e.g., Drews et al. Identification of conserved, functional noncoding elements in the promoter region of the sodium channel gene SCN8A, Mamm Genome (2007) 18:723-731; and Raymond et al. Expression of Alternatively Spliced Sodium Channel a-subunit genes, Journal of Biological Chemistry (2004) 279(44) 46234-46241; the contents of each of which are herein incorporated by reference in their entireties).

Any of promoters taught by the aforementioned Yu, Soderblom, Gill, Husain, Passini, Xu, Drews or Raymond may be used in the present AAV particles described herein.

In some embodiments, the promoter is not cell specific.

In some embodiments, the promoter is a ubiquitin c (UBC) promoter. The UBC promoter may have a size of 300-350 nucleotides. As a non-limiting example, the UBC promoter is 332 nucleotides.

In some embodiments, the promoter is a P-glucuronidase (GUSB) promoter. The GUSB promoter may have a size of 350-400 nucleotides. As a non-limiting example, the GUSB promoter is 378 nucleotides.

In some embodiments, the promoter is a neurofilament light (NFL) promoter. The NFL promoter may have a size of 600-700 nucleotides. As a non-limiting example, the NFL promoter is 650 nucleotides. As a non-limiting example, the construct may be AAV-promoter-CMV/globin intron-modulatory polynucleotide-RBG, where the AAV may be self-complementary and the AAV may be the DJ serotype,

In some embodiments, the promoter is a neurofilament heavy (NFH) promoter. The NFH promoter may have a size of 900-950 nucleotides. As a non-limiting example, the NFH promoter is 920 nucleotides. As a non-limiting example, the construct may be AAV-promoter-CMV/globin intron-modulatory polynucleotide-RBG, where the AAV may be self-complementary and the AAV may be the DJ serotype.

In some embodiments, the promoter is a scn8a promoter. The scn8a promoter may have a size of 450-500 nucleotides. As a non-limiting example, the sen8a promoter is 470 nucleotides. As a non-limiting example, the construct may be AAV-promoter-CMV/globin intron-modulatory polynucleotide-RBG, where the AAV may be self-complementary and the AAV may be the DJ serotype.

In some embodiments, the viral genome comprises a Pol. III promoter.

In some embodiments, the viral genome comprises a P1 promoter,

In some embodiments, the viral genome comprises a FXN promoter.

In some embodiments, the promoter is a phosphoglycerate kinase 1 (PGK) promoter.

In some embodiments, the promoter is a chicken β-actin (CBA) promoter.

In some embodiments, the promoter is a CAG promoter which is a construct comprising the cytomegalovirus (CMV) enhancer fused to the chicken beta-actin (CBA) promoter.

In some embodiments, the promoter is a cytomegalovirus (CMV) promoter.

In some embodiments, the viral genome comprises a H1 promoter.

In some embodiments, the viral genome comprises a U6 promoter.

In some embodiments, the promoter is a liver or a skeletal muscle promoter. Non-limiting examples of liver promoters include human α-1-antitrypsin (hAAT) and thyroxine binding globulin (TBG). Non-limiting examples of skeletal muscle promoters include Desmin, MCK or synthetic C5-12.

In some embodiments, the promoter is an RNA pot III promoter, As a non-limiting example, the RNA pol III promoter is U6. As a non-limiting example, the RNA poi III promoter is H1.

In some embodiments, the viral genome comprises two promoters. As a non-limiting example, the promoters are an EF la promoter and a CMV promoter.

In some embodiments, the viral genome comprises an enhancer element, a promoter and/or a 5′UTR intron. The enhancer element, also referred to herein as an “enhancer,” may be, but is not limited to, a CMV enhancer, the promoter may be, but is not limited to, a CMV, CBA, UBC, GUSB, NSF., Synapsin, MeCP2, and GET promoter and the 5′UTR/intron may be, but is not limited to, SV40, and CBA-MVM. As a non-limiting example, the enhancer, promoter and/or intron used in combination may be: (1) CMV enhancer, CMV promoter, SV40 5′UTR intron; (2) CMV enhancer, CBA promoter, SV 40 5′UTR intron; (3) CMV enhancer, CBA promoter, CBA-MVM 5′UTR intron; (4) UBC promoter; (5) GUSB promoter; (6) NSE promoter; (7) Synapsin promoter; (8) MeCP2 promoter, (9) GFAP promoter, (10) H1 promoter; and (11) U6 promoter.

In some embodiments, the viral genome comprises an engineered promoter.

In another embodiment the viral genome comprises a promoter from a naturally expressed protein.

### Viral Genome Component: Untranslated Regions (UTRs)

By definition, wild type untranslated regions (DTRs) of a gene are transcribed but not translated. Generally, the 5′ UTR starts at the transcription start site and ends at the start codon and the 3′ UTR starts immediately following the stop codon and continues until the termination signal for transcription.

Features typically found in abundantly expressed genes of specific target organs may be engineered into UTRs to enhance the stability and protein production. As a non-limiting example, a 5′ UTR from mRNA normally expressed in the liver (e.g., albumin, serum amyloid A, Apolipoprotein A/B/E, transferrin, alpha fetoprotein, erythropoietin, or Factor VIII) may be used in the viral genomes of the AAV particles of the disclosure to enhance expression in hepatic cell lines or liver.

While not wishing to be bound by theory, wild-type 5′ untranslated regions (UTRs) include features which play roles in translation initiation. Kozak sequences, which are commonly known to be involved in the process by which the ribosome initiates translation of many genes, are usually included in 5′ UTRs. Kozak sequences have the consensus CCR(A/G)CCAUGG, where R is a purine (adenine or guanine) three bases upstream of the start codon (ATG), which is followed by another ‘G’.

In some embodiments, the 5′UTR in the viral genome includes a Kozak sequence.

In some embodiments, the 5′UTR in the viral genome does not include a Kozak sequence.

While not wishing to be bound by theory, wild-type 3′ UTRs are known to have stretches of Adenosines and Uridines embedded therein. These AU rich signatures are particularly prevalent in genes with high rates of turnover. Based on their sequence features and functional properties, the AU rich elements (AREs) can be separated into three classes (Chen et al, 1995, the contents of which are herein incorporated by reference in its entirety): Class I AREs, such as, but not limited to, c-Myc and MyoD, contain several dispersed copies of an AUUUA motif within U-rich regions. Class II AREs, such as, but not limited to, GM-CSF and TNF-a, possess two or more overlapping UUAUUUA(U/A)(U/A) nonamers. Class III ARES, such as, but not limited to, c-Jun and Myogenin, are less well defined. These U rich regions do not contain an AUUUA motif. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

Introduction, removal or modification of 3′ UTR AU rich elements (AREs) can be used to modulate the stability of polynucleotides. When engineering specific polynucleotides, e.g., payload regions of viral genomes, one or more copies of an ARE can be introduced to make polynucleotides less stable and thereby curtail translation and decrease production of the resultant protein. Likewise, AREs can be identified and removed or mutated to increase the intracellular stability and thus increase translation and production of the resultant protein.

In some embodiments, the 3′ UTR of the viral genome may include an oligo(dT) sequence for templated addition of a poly-A tail.

In some embodiments, the viral genome may include at least one miRNA seed, binding site or full sequence. microRNAs (or miRNA or miR) are 19-25 nucleotide noncoding RNAs that bind to the sites of nucleic acid targets and down-regulate gene expression either by reducing nucleic acid molecule stability or by inhibiting translation. A microRNA sequence comprises a “seed” region, i.e., a sequence in the region of positions 2-8 of the mature micro:RNA, which sequence has perfect Watson-Crick complementarity to the miRNA target sequence of the nucleic acid.

in some embodiments, the viral genome may be engineered to include, alter or remove at least one miRNA binding site, sequence or seed region.

Any UTR from any gene known in the art may be incorporated into the viral genome of the AAV particle. These UTRs, or portions thereof, may be placed in the same orientation as in the gene from which they were selected, or they may be altered in orientation or location. In some embodiments, the VTR. used in the viral genome of the AAV particle may be inverted, shortened, lengthened, made with one or more other 5′ UTRs or 3′ UTRs known in the art. As used herein, the term “altered” as it relates to a UTR, means that the UTR has been changed in some way in relation to a reference sequence. For example, a 3′ or 5′ UTR may be altered relative to a wild type or native UTR by the change in orientation or location as taught above or may be altered by the inclusion of additional nucleotides, deletion of nucleotides, swapping or transposition of nucleotides,

In some embodiments, the viral genome of the AAV particle comprises at least one artificial UTRs which is not a variant of a wild type UTR.

In some embodiments, the viral genome of the AAV particle comprises UTRs which have been selected from a family of transcripts whose proteins share a common function, structure, feature or property.

### Viral Genome Component: Polyadenylation Sequence

In some embodiments, the viral genome of the AAV particles of the present disclosure comprise at least one polyadenylation sequence. The viral genome of the AAV particle may comprise a polyadenylation sequence between the 3′ end of the payload coding sequence and the 5′ end of the MR.

In some embodiments, the polyadenylation sequence or “polyA sequence” may range from absent to about 500 nucleotides in length. The polyadenylation sequence may be, but is not limited to, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353. 354, 355, 356, 357, 358, 359, 360. 361, 362, 363, 364, 365, 366, 367, 368, 369, 370, 371, 372, 373, 374, 375, 376, 377, 378, 379. 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, and 500 nucleotides in length,

In some embodiments, the polyadenylation sequence is 50-100 nucleotides in length.

In some embodiments, the polyadenylation sequence is 50-150 nucleotides in length.

In some embodiments, the polyadenylation sequence is 50-160 nucleotides in length.

In some embodiments, the polyadenylation sequence is 50-200 nucleotides in length.

In some embodiments, the polyadenylation sequence is 60-100 nucleotides in length.

in some embodiments, the polyadenylation sequence is 60-150 nucleotides in length.

In some embodiments, the polyadenylation sequence is 60-160 nucleotides in length.

In some embodiments, the polyadenylation sequence is 60-200 nucleotides in length.

In some embodiments, the polyadenylation sequence is 70-100 nucleotides in length.

In some embodiments, the polyadenylation sequence is 70-150 nucleotides in length.

In some embodiments, the polyadenylation sequence is 70-160 nucleotides in length.

In some embodiments, the polyadenylation sequence is 70-200 nucleotides in length.

In some embodiments, the polyadenylation sequence is 80-100 nucleotides in length.

In some embodiments, the polyadenylation sequence is 80-150 nucleotides in length.

In some embodiments, the polyadenylation sequence is 80-160 nucleotides in length.

In some embodiments, the polyadenylation sequence is 80-200 nucleotides in length.

In some embodiments, the polyadenylation sequence is 90-100 nucleotides in length.

In some embodiments, the polyadenylation sequence is 90-150 nucleotides in length.

In some embodiments, the polyadenylation sequence is 90-160 nucleotides in length.

In some embodiments, the polyadenylation sequence is 90-200 nucleotides in length.

In some embodiments, the AAV particle comprises a nucleic acid sequence encoding an siRNA molecule may be located upstream of the polyadenylation sequence in an expression vector. Further, the AAV particle comprises a nucleic acid sequence encoding an siRNA molecule may be located downstream of a promoter such as, but not limited to, CMV, U6, CAG, CBA or a CBA promoter with a SV40 intron or a human beta-globin intron in an expression vector. As a non-limiting example, the AAV particle comprises a nucleic acid sequence encoding an siRNA molecule may be located within 1, 2, 3. 4, 5, 6, 7, 8, 9, 10, 11, 12, 13, 14, 15, 16, 17, 18, 19, 20, 21, 22, 23, 24, 25, 26, 27, 28, 29, 30 or more than 30 nucleotides downstream from the promoter and/or upstream of the polyadenylation sequence in an expression vector. As another non-limiting example, the AAV particle comprises a nucleic acid sequence encoding an siRNA molecule may be located within 1-5, 1-10, 1-15, 1-20, 1-25, 1-30, 5-10, 5-15, 5-20, 5-25, 5-30, 10-15, 10-20, 10-25, 10-30, 15-20, 15-25, 15-30, 20-25, 20-30 or 25-30 nucleotides downstream from the promoter and/or upstream of the polyadenylation sequence in an expression vector. As a non-limiting example, the AAV particle comprises a nucleic acid sequence encoding an siRNA molecule may be located within the first 1%, 2%, 3%, 4%, 5%, 6%, 7%, 8%, 9%, 10%, 15%, 20%, 25% or more than 25% of the nucleotides downstream from the promoter and/or upstream of the polyadenylation sequence in an expression vector, As another non-limiting example, the AAV particle comprises a nucleic acid sequence encoding an siRNA molecule may be located with the first 1-5%, 1-10%, 1-15%, 1-20%, 1-25%, 5-10%, 5-15%, 5-20%, 5-25%, 10-15%, 10-20%, 10-25%, 15-20%, 15-25%, or 20-25% downstream from the promoter and/or upstream of the polyadenylation sequence in an expression vector.

In some embodiments, the AAV particle comprises a rabbit globin polyadenylation (polyA) signal sequence.

In some embodiments, the AAV particle comprises a human growth hormone polyadenylation (polyA) signal sequence.

### Viral Genome Component: Introns

In some embodiments, the payload region comprises at least one element to enhance the expression such as one or more introns or portions thereof. Non-limiting examples of introns include, MVM (67-97 bps), F.IX truncated intron 1 (300 bps), β-globin SD/immunoglobulin heavy chain splice acceptor (250 bps), adenovirus splice donor/immunoglobin splice acceptor (500 bps), SV40 late splice donor/splice acceptor (19S/16S) (180 bps) and hybrid adenovirus splice donor/IgG splice acceptor (230 bps).

In some embodiments, the intron or intron portion may be 100-500 nucleotides in length. The intron may have a length of 80. 90, 100, 110, 120, 130, 140, 150, 160, 170, 171, 172, 173, 174, 175, 176, 177, 178, 179, 180, 190, 200, 210, 220, 230, 240, 250, 260, 270, 280, 290, 300, 310, 320. 330, 340, 350, 360, 370, 380, 390. 400, 410, 420, 430, 440, 450, 460. 470, 480, 490 or 500. The intron may have a length between 80-100, 80-120, 80-140, 80-160, 80-180, 80-200, 80-250, 80-300, 80-350, 80-400, 80-450, 80-500, 200-300, 200-400, 200-500, 300-400, 300-500, or 400-500.

In some embodiments, the AAV viral genome may comprise a promoter such as, but not limited to, CMV or U6. As a non-limiting example, the promoter for the AAV comprising the nucleic acid sequence for the siRNA molecules of the present disclosure is a CMV promoter. As another non-limiting example, the promoter for the AAV comprising the nucleic acid sequence for the siRNA molecules of the disclosure is a U6 promoter.

In some embodiments, the AAV viral genome may comprise a CMV promoter.

In some embodiments, the AAV viral genome may comprise a U6 promoter.

In some embodiments, the AAV viral genome may comprise a CMV and a U6 promoter.

In some embodiments, the AAV viral genome may comprise a H1 promoter.

In some embodiments, the AAV viral genome may comprise a CBA promoter.

In some embodiments, the encoded siRNA molecule may be located downstream of a promoter in an expression vector such as, but not limited to, CMV, U6. H1, CBA, CAG, or a CBA promoter with an intron such as SV40 or others known in the art. Further, the encoded siRNA molecule may also be located upstream of the polyadenylation sequence in an expression vector. As a non-limiting example, the encoded siRNA molecule may be located within 1, 2, 3, 4, 5, 6, 7. 8, 9, 10, 11, 12, 13, 14, 15, 16, 17, 18, 19, 20, 21, 22, 23, 24, 25, 26, 27, 28, 29, 30 or more than 30 nucleotides downstream from the promoter and/or upstream of the polyadenylation sequence in an expression vector. As another non-limiting example, the encoded siRNA molecule may be located within 1-5, 1-10. 1-15, 1-20, 1-25, 1-30, 5-10, 5-15, 5-20, 5-25, 5-30, 10-15, 10-20, 10-25, 10-30, 15-20, 15-25, 15-30.20-25, 20-30 or 25-30 nucleotides downstream from the promoter and/or upstream of the polyadenylation sequence in an expression vector. As a non-limiting example, the encoded siRNA molecule may be located within the first 1%, 2%, 3%, 1%, 5%, 6%, 7%, 8%, 9%, 10%, 15%, 20%, 25% or more than 25% of the nucleotides downstream from the promoter and/or upstream of the polyadenylation sequence in an expression vector. As another non-limiting example, the encoded siRNA molecule may be located with the first 1-5%, 1-10%, 1-15%, 1-20%, 1-25%, 5-10%, 5-15%, 5-20%, 5-25%), 10-15%, 10-20%, 10-25%, 15-20%, 15-25%, or 20-25€N, downstream from the promoter and/or upstream of the polyadenylation sequence in an expression vector.

Viral Genome Component: Filler Sequence

In some embodiments, the viral genome comprises one or more filler sequences.

In some embodiments, the viral genome comprises one or more filler sequences in order to have the length of the viral genome be the optimal size for packaging. As a non-limiting example, the viral genome comprises at least one filler sequence in order to have the length of the viral genome be about 2.3 kb. As a non-limiting example, the viral genome comprises at least one filler sequence in order to have the length of the viral genome be about 4.6 kb.

In some embodiments, the viral genome comprises one or more filler sequences in order to reduce the likelihood that a hairpin structure of the vector genome (e.g., a modulatory polynucleotide described herein) may be read as an inverted terminal repeat (ITR) during expression and/or packaging. As a non-limiting example, the viral genome comprises at least one filler sequence in order to have the length of the viral genome be about 2.3 kb. As a non-limiting example, the viral genome comprises at least one filler sequence in order to have the length of the viral genome be about 4.6 kb.

In some embodiments, the viral genome is a single stranded (ss) viral genome and comprises one or more filler sequences which have a length about between 0.1 kb, 3.8 kb, such as, but not limited to, 0.1 kb, 0.2 kb, 0.3 kb, 0.4 kb, 0.5 kb, 0.6 kb, 0.7 kb, 0.8 kb, 0.9 kb, 1 kb, 1.1 kb, 1.2 kb, 1.3 kb, 1.4 kb, 1.5 kb, 1.6 kb, 1.7 kb, 1.8 kb, 1.9 kb, 2 kb, 2.1 kb, 2.2 kb, 2.3 kb, 2.4 kb, 2.5 kb, 2.6 kb, 2.7 kb, 2.8 kb, 2.9 kb, 3 kb, 3.1 kb, 3.2 kb, 3.3 kb, 3.4 kb, 3.5 kb, 3.6 kb, 3.7 kb, or 3.8 kb, As a non-limiting example, the total length filler sequence in the vector genome is 3.1 kb. As a non-limiting example, the total length filler sequence in the vector genome is 2.7 kb. As a non-limiting example, the total length filler sequence in the vector genome is 0.8 kb. As a non-limiting example, the total length filler sequence in the vector genome is 0.4 kb. As a non-limiting example, the length of each filler sequence in the vector genome is 0.8 kb, As a non-limiting example, the length of each filler sequence in the vector genome is 0.4 kb.

In some embodiments, the viral genome is a self-complementary (sc) viral genome and comprises one or more filler sequences which have a length about between 0.1 kb, 1.5 kb, such as, but not limited to, 0.1 kb, 0.2 kb, 0.3 kb, 0.4 kb, 0.5 kb, 0.6 kb, 0.7 kb, 0.8 kb, 0.9 kb, I kb, 1.1 kb, 1.2 kb, 1.3 kb, 1.4 kb, or 1.5 kb. As a non-limiting example, the total length filler sequence in the vector genome is 0.8 kb. As a non-limiting example, the total length filler sequence in the vector genome is 0.4 kb. As a non-limiting example, the length of each filler sequence in the vector genome is 0.8 kb. As a non-limiting example, the length of each filler sequence in the vector genome is 0.4 kb.

In some embodiments, the viral genome comprises any portion of a filler sequence. The viral genome may comprise 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 99% of a filler sequence.

In some embodiments, the viral genome is a single stranded (ss) viral genome and comprises one or more filler sequences in order to have the length of the viral genome be about 4.6 kb. As a non-limiting example, the viral genome comprises at least one filler sequence and the filler sequence is located 3′ to the 5′ ITR sequence. As a non-limiting example, the viral genome comprises at least one filler sequence and the filler sequence is located 5′ to a promoter sequence. As a non-limiting example, the viral genome comprises at least one filler sequence and the filler sequence is located 3′ to the polyadenylation signal sequence. As a non-limiting example, the viral genome comprises at least one filler sequence and the filler sequence is located 5′ to the 3′ ITR sequence. As a non-limiting example, the viral genome comprises at least one filler sequence, and the filler sequence is located between two intron sequences. As a non-limiting example, the viral genome comprises at least one filler sequence, and the filler sequence is located within an intron sequence. As a non-limiting example, the viral genome comprises two filler sequences, and the first filler sequence is located 3′ to the 5′ ITR sequence and the second filler sequence is located 3′ to the polyadenylation signal sequence. As a non-limiting example, the viral genome comprises two filler sequences, and the first filler sequence is located 5′ to a promoter sequence and the second filler sequence is located 3′ to the polyadenylation signal sequence. As a non-limiting example, the viral genome comprises two filler sequences, and the first filler sequence is located 3′ to the 5′ ITR sequence and the second filler sequence is located 5′ to the 5′ ITR sequence.

In some embodiments, the viral genome is a self-complementary (sc) viral genome and comprises one or more filler sequences in order to have the length of the viral genome be about 2.3 kb. As a non-limiting example, the viral genome comprises at least one filler sequence and the filler sequence is located 3′ to the 5′ ITR sequence. As a non-limiting example, the viral genome comprises at least one filler sequence and the filler sequence is located 5′ to a promoter sequence. As a non-limiting example, the viral genome comprises at least one filler sequence and the filler sequence is located 3′ to the polyadenylation signal sequence. As a non-limiting example, the viral genome comprises at least one filler sequence and the filler sequence is located 5′ to the 3′ ITR sequence. As a non-limiting example, the viral genome comprises at least one filler sequence, and the filler sequence is located between two intron sequences. As a non-limiting example, the viral genome comprises at least one filler sequence, and the filler sequence is located within an intron sequence. As a non-limiting example, the viral genome comprises two filler sequences, and the first filler sequence is located 3′ to the 5′ ITR sequence and the second filler sequence is located 3′ to the polyadenylation signal sequence. As a non-limiting example, the viral genome comprises two filler sequences, and the first filler sequence is located 5′ to a promoter sequence and the second filler sequence is located 3′ to the polyadenylation signal sequence. As a non-limiting example, the viral genome comprises two filler sequences, and the first filler sequence is located 3′ to the 5′ ITR sequence and the second filler sequence is located 5′ to the 5′ ITR sequence.

In some embodiments, the viral genome may comprise one or more filler sequences between one of more regions of the viral genome. In some embodiments, the filler region may be located before a region such as, but not limited to, a payload region, an inverted terminal repeat (ITR), a promoter region, an intron region, an enhancer region, a polyadenylation signal sequence region, a multiple cloning site (MCS) region, and/or an exon region. In some embodiments, the filler region may be located after a region such as, but not limited to, a payload region, an inverted terminal repeat (ITR), a promoter region, an intron region, an enhancer region, a polyadenylation signal sequence region, a multiple cloning site (MCS) region, and/or an exon region. In some embodiments, the filler region may be located before and after a region such as, but not limited to, a payload region, an inverted terminal repeat (ITR), a promoter region, an intron region, an enhancer region, a polyadenylation signal sequence region, a multiple cloning site (MCS) region, and/or an exon region.

In some embodiments, the viral genome may comprise one or more filler sequences which bifurcates at least one region of the viral genome. The bifurcated region of the viral genome may comprise 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 99% of the of the region to the 5′ of the filler sequence region. As a non-limiting example, the filler sequence may bifurcate at least one region so that 10% of the region is located 5′ to the filler sequence and 90% of the region is located 3′ to the filler sequence. As a non-limiting example, the filler sequence may bifurcate at least one region so that 20% of the region is located 5′ to the filler sequence and 80% of the region is located 3′ to the filler sequence. As a non-limiting example, the filler sequence may bifurcate at least one region so that 30% of the region is located 5′ to the filler sequence and 70% of the region is located 3′ to the filler sequence. As a non-limiting example, the filler sequence may bifurcate at least one region so that 40% of the region is located 5′ to the filler sequence and 60% of the region is located 3′ to the filler sequence. As a non-limiting example, the filler sequence may bifurcate at least one region so that 50% of the region is located 5′ to the filler sequence and 50% of the region is located 3′ to the filler sequence. As a non-limiting example, the filler sequence may bifurcate at least one region so that 60% of the region is located 5′ to the filler sequence and 40% of the region is located 3′ to the filler sequence. As a non-limiting example, the filler sequence may bifurcate at least one region so that 70% of the region is located 5′ to the filler sequence and 30% of the region is located 3′ to the filler sequence. As a non-limiting example, the filler sequence may bifurcate at least one region so that 80% of the region is located 5′ to the filler sequence and 20% of the region is located 3′ to the filler sequence. As a non-limiting example, the filler sequence may bifurcate at least one region so that 90% of the region is located 5′ to the filler sequence and 10% of the region is located 3′ to the filler sequence.

In some embodiments, the viral genome comprises a filler sequence after the 5′ ITR.

In some embodiments, the viral genome comprises a filler sequence after the promoter region. In some embodiments, the viral genome comprises a filler sequence after the payload region. In some embodiments, the viral genome comprises a filler sequence after the intron region. In some embodiments, the viral genome comprises a filler sequence after the enhancer region. In some embodiments, the viral genome comprises a filler sequence after the polyadenylation signal sequence region. In some embodiments, the viral genome comprises a filler sequence after the MCS region. In some embodiments, the viral genome comprises a filler sequence after the exon region.

In some embodiments, the viral genome comprises a filler sequence before the promoter region. In some embodiments, the viral genome comprises a filler sequence before the payload region. In some embodiments, the viral genome comprises a filler sequence before the intron region. In some embodiments, the viral genome comprises a filler sequence before the enhancer region. in some embodiments, the viral genome comprises a filler sequence before the polyadenylation signal sequence region. In some embodiments, the viral genome comprises a filler sequence before the MCS region. In some embodiments, the viral genome comprises a filler sequence before the exon region.

In some embodiments, the viral genome comprises a filler sequence before the 3′ ITR.

In some embodiments, a filler sequence may be located between two regions, such as, but not limited to, the 5′ ITR and the promoter region. In some embodiments, a filler sequence may be located between two regions, such as, but not limited to, the 5′ ITR and the payload region. In some embodiments, a filler sequence may be located between two regions, such as, but not limited to, the 5′ FM and the intron region. In some embodiments, a filler sequence may be located between two regions, such as, but not limited to, the 5′ ITR and the enhancer region. In some embodiments, a filler sequence may be located between two regions, such as, but not limited to, the 5′ ITR and the polyadenylation signal sequence region. In some embodiments, a filler sequence may be located between two regions, such as, but not limited to, the 5′ ITR and the MCS region.

In some embodiments, a filler sequence may be located between two regions, such as, but not limited to, the 5′ ITR and the exon region.

In some embodiments, a filler sequence may be located between two regions, such as, but not limited to, the promoter region and the payload region. In some embodiments, a filler sequence may be located between two regions, such as, but not limited to, the promoter region and the intron region. in some embodiments, a filler sequence may be located between two regions, such as, but not limited to, the promoter region and the enhancer region. In some embodiments, a filler sequence may be located between two regions, such as, but not limited to, the promoter region and the polyadenylation signal sequence region. In some embodiments, a filler sequence may be located between two regions, such as, but not limited to, the promoter region and the MCS region. In some embodiments, a filler sequence may be located between two regions, such as, but not limited to, the promoter region and the exon region. In some embodiments, a filler sequence may be located between two regions, such as, but not limited to, the promoter region and the 3′ ITR.

In some embodiments, a filler sequence may be located between two regions, such as, but not limited to, the payload region and the intron region. in some embodiments, a filler sequence may be located between two regions, such as, but not limited to, the payload region and the enhancer region. In some embodiments, a filler sequence may be located between two regions, such as, but not limited to, the payload region and the polyadenylation signal sequence region. in some embodiments, a filler sequence may be located between two regions, such as, but not limited to, the payload region and the MCS region. In some embodiments, a filler sequence may be located between two regions, such as, but not limited to, the payload region and the exon region.

In some embodiments, a filler sequence may be located between two regions, such as, but not limited to, the payload region and the 3′ ITR.

In some embodiments, a filler sequence may be located between two regions, such as, but not limited to, the intron region and the enhancer region. In some embodiments, a filler sequence may be located between two regions, such as, but not limited to, the intron region and the polyadenylation signal sequence region. In some embodiments, a filler sequence may he located between two regions, such as, but not limited to, the intron region and the MCS region. In some embodiments, a filler sequence may be located between two regions, such as, but not limited to, the intron region and the exon region. In some embodiments, a filler sequence may be located between two regions, such as, but not limited to, the intron region and the 3′ ITR. In some embodiments, a filler sequence may be located between two regions, such as, but not limited to, the enhancer region and the polyadenylation signal sequence region. In some embodiments, a filler sequence may be located between two regions, such as, but not limited to, the enhancer region and the MCS region. In some embodiments, a filler sequence may be located between two regions, such as, but not limited to, the enhancer region and the exon region. In some embodiments, a filler sequence may be located between two regions, such as, but not limited to, the enhancer region and the 3′ ITR.

In some embodiments, a filler sequence may be located between two regions, such as, but not limited to, the polyadenylation signal sequence region and the MCS region. In some embodiments, a filler sequence may be located between two regions, such as, but not limited to, the polyadenylation signal sequence region and the exon region. In some embodiments, a filler sequence may be located between two regions, such as, but not limited to, the polyadenylation signal sequence region and the 3′ ITR.

In some embodiments, a filler sequence may be located between two regions, such as, but not limited to, the MCS region and the exon region. In some embodiments, a filler sequence may be located between two regions, such as, but not limited to, the MCS region and the 3′ ITR,

In some embodiments, a filler sequence may be located between two regions, such as, but not limited to, the exon region and the 3′ ITR.

In some embodiments, a viral genome may comprise two filler sequences, the first filler sequence may be located between the 5′ ITR and promoter region, and the second filler sequence may be located between the promoter region and payload region. In some embodiments, a viral genome may comprise two filler sequences, the first filler sequence may be located between the 5′ ITR and promoter region, and the second filler sequence may be located between the promoter region and intron region. In some embodiments, a viral genome may comprise two filler sequences, the first filler sequence may be located between the 5′ ITR and promoter region, and the second filler sequence may be located between the promoter region and enhancer region. In some embodiments, a viral genome may comprise two filler sequences, the first filler sequence may be located between the 5′ ITR and promoter region, and the second filler sequence may be located between the promoter region and polyadenylation signal sequence region. In some embodiments, a viral genome may comprise two filler sequences, the first filler sequence may be located between the 5′ ITR and promoter region, and the second filler sequence may be located between the promoter region and MCS region, In some embodiments, a viral genome may comprise two filler sequences, the first filler sequence may be located between the 5′ ITR and promoter region, and the second filler sequence may be located between the promoter region and exon region. In some embodiments, a viral genome may comprise two filler sequences, the first filler sequence may be located between the 5′ ITR and promoter region, and the second filler sequence may be located between the promoter region and 3′ ITR. In some embodiments, a viral genome may comprise two filler sequences, the first filler sequence may be located between the 5′ ITR and promoter region, and the second filler sequence may be located between the payload region and intron region. In some embodiments, a viral genome may comprise two filler sequences, the first filler sequence may be located between the 5′ ITR and promoter region, and the second filler sequence may be located between the payload region and enhancer region. In some embodiments, a viral genome may comprise two filler sequences, the first filler sequence may be located between the 5′ ITR and promoter region, and the second filler sequence may be located between the payload region and polyadenylation signal sequence region. In some embodiments, a viral genome may comprise two filler sequences, the first filler sequence may be located between the 5′ ITR and promoter region, and the second filler sequence may be located between the payload region and MCS region. In some embodiments, a viral genome may comprise two filler sequences, the first filler sequence may be located between the 5′ ITR and promoter region, and the second filler sequence may be located between the payload. region and exon region. In some embodiments, a viral genome may comprise two filler sequences, the first filler sequence may be located between the 5′ ITR and promoter region, and the second filler sequence may be located between the payload region and 3′ ITR. In some embodiments, a viral genome may comprise two filler sequences, the first filler sequence may be located between the 5′ ITR and promoter region, and the second filler sequence may be located between the intron region and enhancer region. In some embodiments, a viral genome may comprise two filler sequences, the first filler sequence may be located between the 5′ ITR and promoter region, and the second filler sequence may be located between the intron region and polyadenylation signal sequence region. In some embodiments, a viral genome may comprise two filler sequences, the first filler sequence may be located between the 5′ ITR and promoter region, and the second filler sequence may be located between the intron region and MCS region. In some embodiments, a viral genome may comprise two filler sequences, the first filler sequence may be located between the 5′ ITR and promoter region, and the second filler sequence may be located between the intron region and exon region. In some embodiments, a viral genome may comprise two filler sequences, the first filler sequence may be located between the 5′ ITR and promoter region, and the second filler sequence may be located between the intron region and 3′ ITR, In some embodiments, a viral genome may comprise two filler sequences, the first filler sequence may be located between the 5′ ITR and promoter region, and the second filler sequence may be located between the enhancer region and polyadenylation signal sequence region. In some embodiments, a viral genome may comprise two filler sequences, the first filler sequence may be located between the 5′ ITR. and promoter region, and the second filler sequence may be located between the enhancer region and MCS region. In some embodiments, a viral genome may comprise two filler sequences, the first filler sequence may be located between the 5′ ITR and promoter region, and the second filler sequence may be located between the enhancer region and exon region. In some embodiments, a viral genome may comprise two filler sequences, the first filler sequence may be located between the 5′ ITR and promoter region, and the second filler sequence may be located between the enhancer region and 3′ ITR. In some embodiments, a viral genome may comprise two filler sequences, the first filler sequence may be located between the 5′ ITR and promoter region, and the second filler sequence may be located between the polyadenylation signal sequence region and MCS region. In some embodiments, a viral genome may comprise two filler sequences, the first filler sequence may be located between the 5′ ITR and promoter region, and the second filler sequence may be located between the polyadenylation signal sequence region and exon region. In some embodiments, a viral genome may comprise two filler sequences, the first filler sequence may be located between the 5′ ITR and promoter region, and the second filler sequence may be located between the polyadenylation signal sequence region and 3′ ITR. In some embodiments, a viral genome may comprise two filler sequences, the first filler sequence may be located between the 5′ ITR and promoter region, and the second filler sequence may be located between the MCS region and exon region. In some embodiments, a viral genome may comprise two filler sequences, the first filler sequence may be located between the 5′ ITR and promoter region, and the second filler sequence may be located between the MCS region and 3′ ITR. In some embodiments, a viral genome may comprise two filler sequences, the first filler sequence may be located between the 5′ ITR and promoter region, and the second filler sequence may be located between the exon region and 3′ ITR.

In some embodiments, a viral genome may comprise two filler sequences, the first filler sequence may be located between the 5′ ITR and payload region, and the second filler sequence may be located between the promoter region and payload region. In some embodiments, a viral genome may comprise two filler sequences, the first filler sequence may be located between the 5′ ITR and payload region, and the second filler sequence may be located between the promoter region and intron region. In some embodiments, a viral genome may comprise two filler sequences, the first filler sequence may be located between the 5′ ITR and payload region, and the second filler sequence may be located between the promoter region and enhancer region. In some embodiments, a viral genome may comprise two filler sequences, the first filler sequence may be located between the 5′ ITR and payload region, and the second filler sequence may be located between the promoter region and polyadenylation signal sequence region. In some embodiments, a viral genome may comprise two filler sequences, the first filler sequence may be located between the 5′ ITR and payload region, and the second filler sequence may be located between the promoter region and MCS region, In some embodiments, a viral genome may comprise two filler sequences, the first filler sequence may be located between the 5′ ITR and payload region, and the second filler sequence may be located between the promoter region and exon region. In some embodiments, a viral genome may comprise two filler sequences, the first filler sequence may be located between the 5′ ITR and payload region, and the second filler sequence may be located between the promoter region and 3′ ITR. In some embodiments, a viral genome may comprise two filler sequences, the first filler sequence may be located between the 5′ ITR and payload region, and the second filler sequence may be located between the payload region and intron region. In some embodiments, a viral genome may comprise two filler sequences, the first filler sequence may be located between the 5′ ITR and payload region, and the second filler sequence may be located between the payload region and enhancer region. In some embodiments, a viral genome may comprise two filler sequences, the first filler sequence may be located between the 5′ ITR and payload region, and the second filler sequence may be located between the payload region and polyadenylation signal sequence region. In some embodiments, a viral genome may comprise two filler sequences, the first filler sequence may be located between the 5′ ITR and payload region, and the second filler sequence may be located between the payload region and MCS region. In some embodiments, a viral genome may comprise two filler sequences, the first filler sequence may be located between the 5′ ITR and payload region, and the second filler sequence may be located between the payload region and exon region. In some embodiments, a viral genome may comprise two filler sequences, the first filler sequence may be located between the 5′ ITR and payload region, and the second filler sequence may be located between the payload region and 3′ ITR. In some embodiments, a viral genome may comprise two filler sequences, the first filler sequence may be located between the 5′ ITR and payload region, and the second filler sequence may be located between the intron region and enhancer region. In some embodiments, a viral genome may comprise two filler sequences, the first filler sequence may be located between the 5′ ITR and payload region, and the second filler sequence may be located between the intron region and polyadenylation signal sequence region. In some embodiments, a viral genome may comprise two filler sequences, the first filler sequence may be located between the 5′ ITR and payload region, and the second filler sequence may be located between the intron region and MCS region, In some embodiments, a viral genome may comprise two filler sequences, the first filler sequence may be located between the 5′ ITR and payload region, and the second filler sequence may be located between the intron region and exon region. In some embodiments, a viral genome may comprise two filler sequences, the first filler sequence may be located between the 5′ ITR and payload region, and the second filler sequence may be located between the intron region and 3′ ITR. In some embodiments, a viral genome may comprise two filler sequences, the first filler sequence may be located between the 5′ ITR and payload region, and the second filler sequence may be located between the enhancer region and polyadenylation signal sequence region. In some embodiments, a viral genome may comprise two filler sequences, the first filler sequence may be located between the 5′ ITR and payload region, and the second filler sequence may be located between the enhancer region and MCS region. In some embodiments, a viral genome may comprise two filler sequences, the first filler sequence may be located between the 5′ ITR and payload region, and the second filler sequence may be located between the enhancer region and exon region. In some embodiments, a viral genome may comprise two filler sequences, the first filler sequence may be located between the 5′ ITR and payload region, and the second filler sequence may be located between the enhancer region and 3′ ITR. In some embodiments, a viral genome may comprise two filler sequences, the first filler sequence may be located between the 5′ ITR and payload region, and the second filler sequence may be located between the polyadenylation signal sequence region and MCS region. In some embodiments, a viral genome may comprise two filler sequences, the first filler sequence may be located between the 5′ ITR and payload region, and the second filler sequence may be located between the polyadenylation signal sequence region and exon region. In some embodiments, a viral genome may comprise two filler sequences, the first filler sequence may be located between the 5′ ITR and payload region, and the second tiller sequence may be located between the polyadenylation signal sequence region and 3′ ITR. In some embodiments, a viral genome may comprise two filler sequences, the first filler sequence may be located between the 5′ ITR and payload region, and the second filler sequence may be located between the MCS region and exon region. In some embodiments, a viral genome may comprise two filler sequences, the first filler sequence may be located between the 5′ ITR and payload region, and the second filler sequence may he located between the MCS region and 3′ ITR. In some embodiments, a viral genome may comprise two filler sequences, the first filler sequence may be located between the 5′ ITR and payload region, and the second filler sequence may be located between the exon region and 3′ ITR.

In some embodiments, a viral genome may comprise two filler sequences, the first filler sequence may be located between the 5′ ITR and intron region, and the second filler sequence may be located between the promoter region and payload region. In some embodiments, a viral genome may comprise two filler sequences, the first filler sequence may be located between the 5′ ITR and intron region, and the second filler sequence may be located between the promoter region and intron region. In some embodiments, a viral genome may comprise two filler sequences, the first filler sequence may be located between the 5′ ITR and intron region, and the second filler sequence may be located between the promoter region and enhancer region, In some embodiments, a viral genome may comprise two filler sequences, the first filler sequence may be located between the 5′ ITR and intron region, and the second filler sequence may be located between the promoter region and polyadenylation signal sequence region. In some embodiments, a viral genome may comprise two filler sequences, the first filler sequence may be located between the 5′ ITR and intron region, and the second filler sequence may be located between the promoter region and MCS region. In some embodiments, a viral genome may comprise two filler sequences, the first filler sequence may be located between the 5′ ITR and intron region, and the second filler sequence may be located between the promoter region and exon region. In some embodiments, a viral genome may comprise two filler sequences, the first filler sequence may be located between the 5′ ITR and intron region, and the second filler sequence may be located between the promoter region and 3′ ITR. In some embodiments, a viral genome may comprise two filler sequences, the first filler sequence may be located between the 5′ ITR and intron region, and the second filler sequence may be located between the payload region and intron region. In some embodiments, a viral genome may comprise two filler sequences, the first filler sequence may be located between the 5′ ITR and intron region, and the second filler sequence may be located between the payload region and enhancer region. In some embodiments, a viral genome may comprise two filler sequences, the first filler sequence may be located between the 5′ ITR and intron region, and the second filler sequence may be located between the payload region and polyadenylation signal sequence region. In some embodiments, a viral genome may comprise two filler sequences, the first filler sequence may be located between the 5′ ITR and intron region, and the second filler sequence may be located between the payload region and MCS region. In some embodiments, a viral genome may comprise two filler sequences, the first filler sequence may be located between the 5′ ITR and intron region, and the second filler sequence may be located between the payload region and exon region. In some embodiments, a viral genome may comprise two filler sequences, the first filler sequence may be located between the 5′ ITR and intron region, and the second filler sequence may be located between the payload region and 3′ ITR. In some embodiments, a viral genome may comprise two filler sequences, the first filler sequence may be located between the 5′ ITR and intron region, and the second filler sequence may be located between the intron region and enhancer region. In some embodiments, a viral genome may comprise two filler sequences, the first filler sequence may be located between the 5′ ITR and intron region, and the second filler sequence may be located between the intron region and polyadenylation signal sequence region. In some embodiments, a viral genome may comprise two filler sequences, the first filler sequence may be located between the 5′ ITR and intron region, and the second filler sequence may be located between the intron region and MCS region. In some embodiments, a viral genome may comprise two filler sequences, the first filler sequence may be located between the 5′ ITR and intron region, and the second filler sequence may be located between the intron region and exon region. In some embodiments, a viral genome may comprise two filler sequences, the first filler sequence may be located between the 5′ ITR and intron region, and the second filler sequence may be located between the intron region and 3′ ITR. In some embodiments, a viral genome may comprise two filler sequences, the first filler sequence may be located between the 5′ ITR and intron region, and the second filler sequence may be located between the enhancer region and polyadenylation signal sequence region. In some embodiments, a viral genome may comprise two filler sequences, the first filler sequence may be located between the 5′ ITR and intron region, and the second filler sequence may be located between the enhancer region and MCS region. In some embodiments, a viral genome may comprise two filler sequences, the first filler sequence may be located between the 5′ ITR and intron region, and the second filler sequence may be located between the enhancer region and exon region. In some embodiments, a viral genome may comprise two filler sequences, the first filler sequence may be located between the 5′ ITR and intron region, and the second filler sequence may be located between the enhancer region and 3′ ITR. In some embodiments, a viral genome may comprise two filler sequences, the first filler sequence may he located between the 5′ ITR and intron region, and the second filler sequence may be located between the polyadenylation signal sequence region and MCS region. In some embodiments, a viral genome may comprise two filler sequences, the first filler sequence may be located between the 5′ ITR and intron region, and the second filler sequence may be located between the polyadenylation signal sequence region and exon region. In some embodiments, a viral genome may comprise two filler sequences, the first filler sequence may be located between the 5′ ITR and intron region, and the second filler sequence may be located between the polyadenylation signal sequence region and 3′ ITR. In some embodiments, a viral genome may comprise two filler sequences, the first filler sequence may be located between the 5′ ITR and intron region, and the second filler sequence may be located between the MCS region and exon region. In some embodiments, a viral genome may comprise two filler sequences, the first filler sequence may be located between the 5′ ITR and intron region, and the second filler sequence may be located between the MCS region and 3′ ITR. In some embodiments, a viral genome may comprise two filler sequences, the first filler sequence may be located between the 5′ ITR and intron region, and the second filler sequence may be located between the exon region and 3′ ITR.

In some embodiments, a viral genome may comprise two filler sequences, the first filler sequence may be located between the 5′ ITR and enhancer region, and the second filler sequence may be located between the promoter region and payload region. In some embodiments, a viral genome may comprise two filler sequences, the first filler sequence may be located between the 5′ ITR and enhancer region, and the second filler sequence may be located between the promoter region and intron region. In some embodiments, a viral genome may comprise two filler sequences, the first filler sequence may be located between the 5′ ITR and enhancer region, and the second filler sequence may be located between the promoter region and enhancer region. In some embodiments, a viral genome may comprise two filler sequences, the first filler sequence may be located between the 5′ ITR and enhancer region, and the second filler sequence may be located between the promoter region and polyadenylation signal sequence region. In some embodiments, a viral genome may comprise two filler sequences, the first filler sequence may be located between the 5′ ITR and enhancer region, and the second filler sequence may be located between the promoter region and MCS region. In some embodiments, a viral genome may comprise two filler sequences, the first filler sequence may be located between the 5′ ITR and enhancer region, and the second filler sequence may be located between the promoter region and exon region. In some embodiments, a viral genome may comprise two filler sequences, the first filler sequence may be located between the 5′ ITR and enhancer region, and the second filler sequence may be located between the promoter region and 3′ ITR. In some embodiments, a viral genome may comprise two filler sequences, the first filler sequence may be located between the 5′ ITR and enhancer region, and the second filler sequence may be located. between the payload region and intron region. In some embodiments, a viral genome may comprise two filler sequences, the first filler sequence may be located between the 5′ ITR and enhancer region, and the second filler sequence may be located between the payload region and enhancer region. In some embodiments, a viral genome may comprise two filler sequences, the first filler sequence may be located between the 5′ ITR and enhancer region, and the second filler sequence may be located between the payload region and polyadenylation signal sequence region, In some embodiments, a viral genome may comprise two filler sequences, the first filler sequence may be located between the 5′ ITR, and enhancer region, and the second filler sequence may be located between the payload region and MCS region. In some embodiments, a viral genome may comprise two filler sequences, the first filler sequence may be located between the 5′ ITR and enhancer region, and the second filler sequence may be located between the payload region and exon region. In some embodiments, a viral genome may comprise two filler sequences, the first filler sequence may be located between the 5′ ITR and enhancer region, and the second filler sequence may be located between the payload region and 3′ ITR. In some embodiments, a viral genome may comprise two filler sequences, the first filler sequence may be located between the 5′ ITR and enhancer region, and the second filler sequence may be located between the intron region and enhancer region. In some embodiments, a viral genome may comprise two filler sequences, the first filler sequence may be located between the 5′ ITR and enhancer region, and the second filler sequence may be located between the intron region and polyadenylation signal sequence region. In some embodiments, a viral genome may comprise two filler sequences, the first filler sequence may be located between the 5′ ITR and enhancer region, and the second filler sequence may be located between the intron region and MCS region. In some embodiments, a viral genome may comprise two filler sequences, the first filler sequence may be located between the 5′ ITR and enhancer region, and the second filler sequence may be located between the intron region and exon region. In some embodiments, a viral genome may comprise two filler sequences, the first filler sequence may be located between the 5′ ITR and enhancer region, and the second filler sequence may be located between the intron region and 3′ ITR. In some embodiments, a viral genome may comprise two filler sequences, the first filler sequence may be located between the 5′ ITR and enhancer region, and the second filler sequence may be located between the enhancer region and polyadenylation signal sequence region. In some embodiments, a viral genome may comprise two filler sequences, the first filler sequence may be located between the 5′ ITR and enhancer region, and the second filler sequence may be located between the enhancer region and MCS region. In some embodiments, a viral genome may comprise two filler sequences, the first filler sequence may be located between the 5′ ITR and enhancer region, and the second filler sequence may be located between the enhancer region and exon region. In some embodiments, a viral genome may comprise two filler sequences, the first filler sequence may be located between the 5′ ITR and enhancer region, and the second filler sequence may be located between the enhancer region and 3′ ITR. In some embodiments, a viral genome may comprise two filler sequences, the first filler sequence may be located between the 5′ ITR and enhancer region, and the second filler sequence may be located between the polyadenylation signal sequence region and MCS region. In some embodiments, a viral genome may comprise two filler sequences, the first filler sequence may be located between the 5′ ITR and enhancer region, and the second filler sequence may be located between the polyadenylation signal sequence region and exon region. In some embodiments, a viral genome may comprise two filler sequences, the first filler sequence may be located between the 5′ ITR and enhancer region, and the second filler sequence may be located between the polyadenylation signal sequence region and 3′ ITR. In some embodiments, a viral genome may comprise two filler sequences, the first filler sequence may be located between the 5′ ITR and enhancer region, and the second filler sequence may be located between the MCS region and exon region. In some embodiments, a viral genome may comprise two filler sequences, the first filler sequence may be located between the 5′ ITR and enhancer region, and the second filler sequence may be located between the MCS region and 3′ ITR. In some embodiments, a viral genome may comprise two filler sequences, the first filler sequence may be located between the 5′ ITR and enhancer region, and the second filler sequence may be located between the exon region and 3′ ITR.

In some embodiments, a viral genome may comprise two filler sequences, the first filler sequence may be located between the 5′ ITR and polyadenylation signal sequence region, and the second filler sequence may be located between the promoter region and payload region. In some embodiments, a viral genome may comprise two filler sequences, the first filler sequence may be located between the 5′ ITR and polyadenylation signal sequence region, and the second filler sequence may be located between the promoter region and intron region. In some embodiments, a viral genome may comprise two filler sequences, the first filler sequence may be located between the 5′ ITR and polyadenylation signal sequence region, and the second filler sequence may be located between the promoter region and enhancer region. In some embodiments, a viral genome may comprise two filler sequences, the first filler sequence may be located between the 5′ ITR and polyadenylation signal sequence region, and the second filler sequence may be located between the promoter region and polyadenylation signal sequence region. In some embodiments, a viral genome may comprise two filler sequences, the first filler sequence may be located between the 5′ ITR and polyadenylation signal sequence region, and the second filler sequence may be located between the promoter region and MCS region. In some embodiments, a viral genome may comprise two filler sequences, the first filler sequence may be located between the 5′ ITR and polyadenylation signal sequence region, and the second filler sequence may be located between the promoter region and exon region. In some embodiments, a viral genome may comprise two filler sequences, the first filler sequence may be located between the 5′ ITR and polyadenylation signal sequence region, and the second filler sequence may be located between the promoter region and 3′ ITR. In some embodiments, a viral genome may comprise two filler sequences, the first filler sequence may be located between the 5′ ITR and polyadenylation signal sequence region, and the second filler sequence may be located between the payload region and intron region. In some embodiments, a viral genome may comprise two filler sequences, the first filler sequence may be located between the 5′ ITR and polyadenylation signal sequence region, and the second filler sequence may be located between the payload region and enhancer region. In some embodiments, a viral genome may comprise two filler sequences, the first filler sequence may be located between the 5′ ITR and polyadenylation signal sequence region, and the second filler sequence may be located between the payload region and polyadenylation signal sequence region. In some embodiments, a viral genome may comprise two filler sequences, the first filler sequence may be located between the 5′ ITR and polyadenylation signal sequence region, and the second filler sequence may be located between the payload region and MCS region. In some embodiments, a viral genome may comprise two filler sequences, the first filler sequence may be located between the 5′ ITR and polyadenylation signal sequence region, and the second filler sequence may be located between the payload region and exon region. In some embodiments, a viral genome may comprise two filler sequences, the first filler sequence may be located between the 5′ ITR and polyadenylation signal sequence region, and the second filler sequence may be located between the payload region and 3′ ITR, In some embodiments, a viral genome may comprise two filler sequences, the first filler sequence may be located between the 5′ ITR and polyadenylation signal sequence region, and the second filler sequence may be located between the intron region and enhancer region. In some embodiments, a viral genome may comprise two filler sequences, the first filler sequence may be located between the 5′ ITR and polyadenylation signal sequence region, and the second filler sequence may be located between the intron region and polyadenylation signal sequence region. In some embodiments, a viral genome may comprise two filler sequences, the first filler sequence may be located between the 5′ ITR and polyadenylation signal sequence region, and the second filler sequence may be located between the intron region and MCS region. In some embodiments, a viral genome may comprise two filler sequences, the first filler sequence may be located between the 5′ ITR and polyadenylation signal sequence region, and the second filler sequence may be located between the intron region and exon region. In some embodiments, a viral genome may comprise two filler sequences, the first filler sequence may be located between the 5′ ITR and polyadenylation signal sequence region, and the second filler sequence may be located between the intron region and 3′ ITR. In some embodiments, a viral genome may comprise two filler sequences, the first filler sequence may be located between the 5′ ITR and polyadenylation signal sequence region, and the second filler sequence may be located between the enhancer region and polyadenylation signal sequence region. In some embodiments, a viral genome may comprise two filler sequences, the first filler sequence may be located between the 5′ ITR and polyadenylation signal sequence region, and the second filler sequence may be located between the enhancer region and MCS region. In some embodiments, a viral genome may comprise two filler sequences, the first filler sequence may be located between the 5′ ITR and polyadenylation signal sequence region, and the second filler sequence may be located between the enhancer region and exon region. In some embodiments, a viral genome may comprise two filler sequences, the first filler sequence may be located between the 5′ ITR and polyadenylation signal sequence region, and the second filler sequence may be located between the enhancer region and 3′ ITR. In some embodiments, a viral genome may comprise two filler sequences, the first filler sequence may be located between the 5′ ITR and polyadenylation signal sequence region, and the second filler sequence may be located between the polyadenylation signal sequence region and MCS region. In some embodiments, a viral genome may comprise two filler sequences, the first filler sequence may be located between the 5′ ITR and polyadenylation signal sequence region, and the second filler sequence may be located between the polyadenylation signal sequence region and exon region. In some embodiments, a viral genome may comprise two filler sequences, the first filler sequence may be located between the 5′ ITR and polyadenylation signal sequence region, and the second filler sequence may be located between the polyadenylation signal sequence region and 3′ ITR. In some embodiments, a viral genome may comprise two filler sequences, the first filler sequence may be located between the 5′ ITR and polyadenylation signal sequence region, and the second filler sequence may be located between the MCS region and exon region. In some embodiments, a viral genome may comprise two filler sequences, the first filler sequence may be located between the 5′ ITR and polyadenylation signal sequence region, and the second filler sequence may be located between the MCS region and 3′ ITR. In some embodiments, a viral genome may comprise two filler sequences, the first filler sequence may be located between the 5′ ITR and polyadenylation signal sequence region, and the second filler sequence may be located between the exon region and 3′ ITR.

In some embodiments, a viral genome may comprise two filler sequences, the first filler sequence may be located between the 5′ ITR and MCS region, and the second filler sequence may be located between the promoter region and payload region. In some embodiments, a viral genome may comprise two filler sequences, the first filler sequence may be located between the 5′ ITR and MCS region, and the second filler sequence may be located between the promoter region and intron region. In some embodiments, a viral genome may comprise two filler sequences, the first filler sequence may be located between the 5′ ITR and MCS region, and the second filler sequence may be located between the promoter region and enhancer region. In some embodiments, a viral genome may comprise two filler sequences, the first filler sequence may be located between the 5′ ITR and MCS region, and the second filler sequence may be located between the promoter region and polyadenylation signal sequence region. In some embodiments, a viral genome may comprise two filler sequences, the first filler sequence may be located between the 5′ ITR, and MCS region, and the second filler sequence may be located between the promoter region and MCS region. In some embodiments, a viral genome may comprise two filler sequences, the first filler sequence may be located between the 5′ ITR and MCS region, and the second filler sequence may be located between the promoter region and exon region. In some embodiments, a viral genome may comprise two filler sequences, the first filler sequence may be located between the 5′ ITR and MCS region, and the second filler sequence may be located between the promoter region and 3′ ITR, In some embodiments, a viral genome may comprise two filler sequences, the first filler sequence may be located between the 5′ ITR and MCS region, and the second filler sequence may be located between the payload region and intron region. In some embodiments, a viral genome may comprise two filler sequences, the first filler sequence may be located between the 5′ ITR and MCS region, and the second filler sequence may be located between the payload region and enhancer region. In some embodiments, a viral genome may comprise two filler sequences, the first filler sequence may be located between the 5′ ITR and MCS region, and the second filler sequence may be located between the payload region and polyadenylation signal sequence region. In some embodiments, a viral genome may comprise two filler sequences, the first filler sequence may be located between the 5′ ITR and MCS region, and the second filler sequence may be located between the payload region and MCS region. In some embodiments, a viral genome may comprise two filler sequences, the first filler sequence may be located between the 5′ ITR and MCS region, and the second filler sequence may be located between the payload region and exon region. In some embodiments, a viral genome may comprise two filler sequences, the first filler sequence may be located between the 5′ ITR. and MCS region, and the second filler sequence may be located between the payload region and 3′ ITR. In some embodiments, a viral genome may comprise two filler sequences, the first filler sequence may be located between the 5′ ITR and MCS region, and the second filler sequence may be located between the intron region and enhancer region. In some embodiments, a viral genome may comprise two filler sequences, the first filler sequence may be located between the 5′ ITR and MCS region, and the second filler sequence may be located between the intron region and polyadenylation signal sequence region. In some embodiments, a viral genome may comprise two filler sequences, the first filler sequence may be located between the 5′ ITR and MCS region, and the second filler sequence may be located between the intron region and MCS region. In some embodiments, a viral genome may comprise two filler sequences, the first filler sequence may be located between the 5′ ITR and MCS region, and the second filler sequence may be located between the intron region and exon region. In some embodiments, a viral genome may comprise two filler sequences, the first filler sequence may be located between the 5′ ITR and MCS region, and the second filler sequence may be located between the intron region and 3′ ITR. In some embodiments, a viral genome may comprise two filler sequences, the first filler sequence may be located between the 5′ ITR and MCS region, and the second filler sequence may be located between the enhancer region and polyadenylation signal sequence region. In some embodiments, a viral genome may comprise two filler sequences, the first filler sequence may be located between the 5′ ITR and MCS region, and the second filler sequence may be located between the enhancer region and MCS region. In some embodiments, a viral genome may comprise two filler sequences, the first filler sequence may be located between the 5′ ITR and MCS region, and the second filler sequence may be located between the enhancer region and exon region. In some embodiments, a viral genome may comprise two filler sequences, the first filler sequence may be located between the 5′ ITR and MCS region, and the second filler sequence may be located between the enhancer region and 3′ ITR. In some embodiments, a viral genome may comprise two filler sequences, the first filler sequence may be located between the 5′ ITR and MCS region, and the second filler sequence may be located between the polyadenylation signal sequence region and MCS region. In some embodiments, a viral genome may comprise two filler sequences, the first filler sequence may be located between the 5′ ITR and MCS region, and the second filler sequence may be located between the polyadenylation signal sequence region and exon region. In some embodiments, a viral genome may comprise two filler sequences, the first filler sequence may be located between the 5′ ITR. and MCS region, and the second filler sequence may be located between the polyadenylation signal sequence region and 3′ ITR. In some embodiments, a viral genome may comprise two filler sequences, the first filler sequence may be located between the 5′ ITR and MCS region, and the second filler sequence may be located between the MCS region and exon region. In some embodiments, a viral genome may comprise two filler sequences, the first filler sequence may be located between the 5′ ITR and MCS region, and the second filler sequence may be located between the MCS region and 3′ ITR. In some embodiments, a viral genome may comprise two filler sequences, the first filler sequence may be located between the 5′ ITR and MCS region, and the second filler sequence may be located between the exon region and 3′ ITR.

In some embodiments, a viral genome may comprise two filler sequences, the first filler sequence may be located between the 5′ ITR and exon region, and the second filler sequence may be located between the promoter region and payload region. In some embodiments, a viral genome may comprise two filler sequences, the first filler sequence may be located between the 5′ ITR and exon region, and the second filler sequence may be located between the promoter region and intron region. In some embodiments, a viral genome may comprise two filler sequences, the first filler sequence may be located between the 5′ ITR and exon region, and the second filler sequence may be located between the promoter region and enhancer region. In some embodiments, a viral genome may comprise two filler sequences, the first filler sequence may be located between the 5′ ITR and exon region, and the second filler sequence may be located between the promoter region and polyadenylation signal sequence region. In some embodiments, a viral genome may comprise two filler sequences, the first filler sequence may be located between the 5′ ITR and exon region, and the second filler sequence may be located between the promoter region and MCS region. In some embodiments, a viral genome may comprise two filler sequences, the first filler sequence may be located between the 5′ ITR and exon region, and the second filler sequence may be located between the promoter region and exon region. In some embodiments, a viral genome may comprise two filler sequences, the first filler sequence may be located between the 5′ ITR and exon region, and the second filler sequence may be located between the promoter region and 3′ ITR. In some embodiments, a viral genome may comprise two filler sequences, the first filler sequence may be located between the 5′ ITR and exon region, and the second filler sequence may be located between the payload region and intron region. In some embodiments, a viral genome may comprise two filler sequences, the first filler sequence may be located between the 5′ ITR and exon region, and the second filler sequence may be located between the payload region and enhancer region. In some embodiments, a viral genome may comprise two filler sequences, the first filler sequence may be located between the 5′ ITR and exon region, and the second filler sequence may be located between the payload region and polyadenylation signal sequence region. In some embodiments, a viral genome may comprise two filler sequences, the first filler sequence may be located between the 5′ ITR and exon region, and the second filler sequence may be located between the payload region and MCS region. In some embodiments, a viral genome may comprise two filler sequences, the first filler sequence may be located between the 5′ ITR and exon region, and the second filler sequence may be located between the payload region and exon region. In some embodiments, a viral genome may comprise two filler sequences, the first filler sequence may be located between the 5′ ITR and exon region, and the second filler sequence may be located between the payload region and 3′ ITR. In some embodiments, a viral genome may comprise two filler sequences, the first filler sequence may be located between the 5′ ITR and exon region, and the second filler sequence may be located between the intron region and enhancer region. In some embodiments, a viral genome may comprise two filler sequences, the first filler sequence may be located between the 5′ ITR and exon region, and the second filler sequence may be located between the intron region and polyadenylation signal sequence region. In some embodiments, a viral genome may comprise two filler sequences, the first filler sequence may be located between the 5′ ITR and exon region, and the second filler sequence may be located between the intron region and MCS region, In some embodiments, a viral genome may comprise two filler sequences, the first filler sequence may be located between the 5′ ITR and exon region, and the second filler sequence may be located between the intron region and exon region. In some embodiments, a viral genome may comprise two filler sequences, the first filler sequence may be located between the 5′ ITR and exon region, and the second filler sequence may be located between the intron region and 3′ ITR. In some embodiments, a viral genome may comprise two filler sequences, the first filler sequence may be located between the 5′ ITR and exon region, and the second filler sequence may be located between the enhancer region and polyadenylation signal sequence region. In some embodiments, a viral genome may comprise two filler sequences, the first filler sequence may be located between the 5′ ITR and exon region, and the second filler sequence may be located between the enhancer region and MCS region. in some embodiments, a viral genome may comprise two filler sequences, the first filler sequence may be located between the 5′ ITR and exon region, and the second filler sequence may be located between the enhancer region and exon region. In some embodiments, a viral genome may comprise two filler sequences, the first filler sequence may be located between the 5′ ITR and exon region, and the second filler sequence may be located between the enhancer region and 3′ ITR. In some embodiments, a viral genome may comprise two filler sequences, the first filler sequence may be located between the 5′ ITR and exon region, and the second filler sequence may be located between the polyadenylation signal sequence region and MCS region. In some embodiments, a viral genome may comprise two filler sequences, the first filler sequence may be located between the 5′ ITR and exon region, and the second filler sequence may be located between the polyadenylation signal sequence region and exon region. In some embodiments, a viral genome may comprise two filler sequences, the first filler sequence may be located between the 5′ ITR and exon region, and the second filler sequence may be located between the polyadenylation signal sequence region and 3′ ITR. In some embodiments, a viral genome may comprise two filler sequences, the first filler sequence may be located between the 5′ ITR and exon region, and the second filler sequence may be located between the MCS region and exon region. In some embodiments, a viral genome may comprise two filler sequences, the first filler sequence may be located between the 5′ ITR and exon region, and the second filler sequence may be located between the MCS region and 3′ ITR. In some embodiments, a viral genome may comprise two filler sequences, the first filler sequence may be located between the 5′ ITR and exon region, and the second filler sequence may be located between the exon region and 3′ ITR.

In some embodiments, a viral genome may comprise two filler sequences, the first filler sequence may be located between the promoter region and payload region, and the second filler sequence may be located between the payload region and intron region. In some embodiments, a viral genome may comprise two filler sequences, the first filler sequence may be located between the promoter region and payload region, and the second filler sequence may be located between the payload region and enhancer region. In some embodiments, a viral genome may comprise two filler sequences, the first filler sequence may be located between the promoter region and payload region, and the second filler sequence may be located between the payload region and polyadenylation signal sequence region. In some embodiments, a viral genome may comprise two filler sequences, the first filler sequence may be located between the promoter region and payload region, and the second filler sequence may be located between the payload region and MCS region. In some embodiments, a viral genome may comprise two filler sequences, the first filler sequence may be located between the promoter region and payload region, and the second filler sequence may be located between the payload region and exon region. In some embodiments, a viral genome may comprise two filler sequences, the first filler sequence may be located between the promoter region and payload region, and the second filler sequence may be located between the payload region and 3′ ITR. In some embodiments, a viral genome may comprise two filler sequences, the first filler sequence may be located between the promoter region and payload region, and the second filler sequence may be located between the intron region and enhancer region. In some embodiments, a viral genome may comprise two filler sequences, the first filler sequence may be located between the promoter region and payload region, and the second filler sequence may be located between the intron region and polyadenylation signal sequence region. In some embodiments, a viral genome may comprise two filler sequences, the first filler sequence may be located between the promoter region and payload region, and the second filler sequence may be located between the intron region and MCS region. In some embodiments, a viral genome may comprise two filler sequences, the first filler sequence may be located between the promoter region and payload region, and the second filler sequence may be located between the intron region and exon region. In some embodiments, a viral genome may comprise two filler sequences, the first filler sequence may be located between the promoter region and payload region, and the second filler sequence may be located between the intron region and 3′ ITR. In some embodiments, a viral genome may comprise two filler sequences, the first filler sequence may be located between the promoter region and payload region, and the second filler sequence may be located between the enhancer region and polyadenylation signal sequence region. In some embodiments, a viral genome may comprise two filler sequences, the first filler sequence may be located between the promoter region and payload region, and the second filler sequence may be located between the enhancer region and MCS region. In some embodiments, a viral genome may comprise two filler sequences, the first filler sequence may be located between the promoter region and payload region, and the second filler sequence may be located between the enhancer region and exon region. In some embodiments, a viral genome may comprise two filler sequences, the first filler sequence may be located between the promoter region and payload region, and the second filler sequence may be located between the enhancer region and 3′ ITR. In some embodiments, a viral genome may comprise two filler sequences, the first filler sequence may be located between the promoter region and payload region, and the second filler sequence may be located between the polyadenylation signal sequence region and MCS region. In some embodiments, a viral genome may comprise two filler sequences, the first filler sequence may be located between the promoter region and payload region, and the second filler sequence may be located between the polyadenylation signal sequence region and exon region. In some embodiments, a viral genome may comprise two filler sequences, the first filler sequence may be located between the promoter region and payload region, and the second filler sequence may be located between the polyadenylation signal sequence region and 3′ ITR. In some embodiments, a viral genome may comprise two filler sequences, the first filler sequence may be located between the promoter region and payload region, and the second filler sequence may be located between the MCS region and exon region. In some embodiments, a viral genome may comprise two filler sequences, the first filler sequence may be located between the promoter region and payload region, and the second filler sequence may be located between the MCS region and 3′ ITR. In some embodiments, a viral genome may comprise two filler sequences, the first filler sequence may be located between the promoter region and payload region, and the second filler sequence may be located between the exon region and 3′ ITR.

In some embodiments, a viral genome may comprise two filler sequences, the first filler sequence may be located between the promoter region and intron region, and the second filler sequence may be located between the payload region and intron region. In some embodiments, a viral genome may comprise two filler sequences, the first filler sequence may be located between the promoter region and intron region, and the second filler sequence may be located between the payload region and enhancer region. In some embodiments, a viral genome may comprise two filler sequences, the first filler sequence may be located between the promoter region and intron region, and the second filler sequence may be located between the payload region and polyadenylation signal sequence region. In some embodiments, a viral genome may comprise two filler sequences, the first filler sequence may be located between the promoter region and intron region, and the second filler sequence may be located between the payload region and MCS region. In some embodiments, a viral genome may comprise two filler sequences, the first filler sequence may be located between the promoter region and intron region, and the second filler sequence may be located between the payload region and exon region. In some embodiments, a viral genome may comprise two filler sequences, the first filler sequence may be located between the promoter region and intron region, and the second filler sequence may be located between the payload region and 3′ ITR. In some embodiments, a viral genome may comprise two filler sequences, the first filler sequence may be located between the promoter region and intron region, and the second filler sequence may be located between the intron region and enhancer region. In some embodiments, a viral genome may comprise two filler sequences, the first filler sequence may be located between the promoter region and intron region, and the second filler sequence may be located between the intron region and polyadenylation signal sequence region. in some embodiments, a viral genome may comprise two filler sequences, the first filler sequence may be located between the promoter region and intron region, and the second filler sequence may be located between the intron region and MCS region. In some embodiments, a viral genome may comprise two filler sequences, the first filler sequence may be located between the promoter region and intron region, and the second filler sequence may be located between the intron region and exon region. In some embodiments, a viral genome may comprise two filler sequences, the first filler sequence may be located between the promoter region and intron region, and the second filler sequence may be located between the intron region and 3′ ITR. In some embodiments, a viral genome may comprise two filler sequences, the first filler sequence may be located between the promoter region and intron region, and the second filler sequence may be located between the enhancer region and polyadenylation signal sequence region. In some embodiments, a viral genome may comprise two filler sequences, the first filler sequence may be located between the promoter region and intron region, and the second filler sequence may be located between the enhancer region and MCS region. In some embodiments, a viral genome may comprise two filler sequences, the first filler sequence may be located between the promoter region and intron region, and the second filler sequence may be located between the enhancer region and exon region. In some embodiments, a viral genome may comprise two filler sequences, the first filler sequence may be located between the promoter region and intron region, and the second filler sequence may be located between the enhancer region and 3′ ITR. In some embodiments, a viral genome may comprise two filler sequences, the first filler sequence may be located between the promoter region and intron region, and the second filler sequence may be located between the polyadenylation signal sequence region and MCS region. In some embodiments, a viral genome may comprise two filler sequences, the first filler sequence may be located between the promoter region and intron region, and the second filler sequence may be located between the polyadenylation signal sequence region and exon region. In some embodiments, a viral genome may comprise two filler sequences, the first filler sequence may be located between the promoter region and intron region, and the second filler sequence may be located between the polyadenylation signal sequence region and 3′ ITR. In some embodiments, a viral genome may comprise two filler sequences, the first filler sequence may be located between the promoter region and intron region, and the second filler sequence may be located between the MCS region and exon region. In some embodiments, a viral genome may comprise two filler sequences, the first filler sequence may be located between the promoter region and intron region, and the second filler sequence may be located between the MCS region and 3′ ITR. In some embodiments, a viral genome may comprise two filler sequences, the first filler sequence may be located between the promoter region and intron region, and the second filler sequence may be located between the exon region and 3′ ITR.

In some embodiments, a viral genome may comprise two filler sequences, the first filler sequence may be located between the promoter region and enhancer region, and the second filler sequence may be located between the payload region and intron region. In some embodiments, a viral genome may comprise two filler sequences, the first filler sequence may be located between the promoter region and enhancer region, and the second filler sequence may be located between the payload region and enhancer region. In some embodiments, a viral genome may comprise two filler sequences, the first filler sequence may be located between the promoter region and enhancer region, and the second filler sequence may be located between the payload region and polyadenylation signal sequence region. In some embodiments, a viral genome may comprise two filler sequences, the first filler sequence may be located between the promoter region and enhancer region, and the second filler sequence may be located between the payload region and MCS region. In some embodiments, a viral genome may comprise two filler sequences, the first filler sequence may be located between the promoter region and enhancer region, and the second filler sequence may be located between the payload region and exon region. In some embodiments, a viral genome may comprise two filler sequences, the first filler sequence may be located between the promoter region and enhancer region, and the second filler sequence may be located between the payload region and 3′ ITR. In some embodiments, a viral genome may comprise two filler sequences, the first filler sequence may be located between the promoter region and enhancer region, and the second filler sequence may be located between the intron region and enhancer region. In some embodiments, a viral genome may comprise two filler sequences, the first filler sequence may be located between the promoter region and enhancer region, and the second filler sequence may be located between the intron region and polyadenylation signal sequence region. In some embodiments, a viral genome may comprise two filler sequences, the first filler sequence may be located between the promoter region and enhancer region, and the second filler sequence may be located between the intron region and MCS region. In some embodiments, a viral genome may comprise two filler sequences, the first filler sequence may be located between the promoter region and enhancer region, and the second filler sequence may be located between the intron region and exon region. In some embodiments, a viral genome may comprise two filler sequences, the first filler sequence may be located between the promoter region and enhancer region, and the second filler sequence may be located between the intron region and 3′ ITR. In some embodiments, a viral genome may comprise two filler sequences, the first filler sequence may be located between the promoter region and enhancer region, and the second filler sequence may be located between the enhancer region and polyadenylation signal sequence region. In some embodiments, a viral genome may comprise two filler sequences, the first filler sequence may be located between the promoter region and enhancer region, and the second filler sequence may be located between the enhancer region and MCS region. In some embodiments, a viral genome may comprise two filler sequences, the first filler sequence may be located between the promoter region and enhancer region, and the second filler sequence may be located between the enhancer region and exon region. In some embodiments, a viral genome may comprise two filler sequences, the first filler sequence may be located between the promoter region and enhancer region, and the second filler sequence may be located between the enhancer region and 3′ ITR. In some embodiments, a viral genome may comprise two filler sequences, the first filler sequence may be located between the promoter region and enhancer region, and the second filler sequence may be located between the polyadenylation signal sequence region and MCS region. In some embodiments, a viral genome may comprise two filler sequences, the first filler sequence may be located between the promoter region and enhancer region, and the second filler sequence may be located between the polyadenylation signal sequence region and exon region. In some embodiments, a viral genome may comprise two filler sequences, the first filler sequence may be located between the promoter region and enhancer region, and the second filler sequence may be located between the polyadenylation signal sequence region and 3′ ITR. In some embodiments, a viral genome may comprise two filler sequences, the first filler sequence may be located between the promoter region and enhancer region, and the second filler sequence may be located between the MCS region and exon region. In some embodiments, a viral genome may comprise two filler sequences, the first filler sequence may be located between the promoter region and enhancer region, and the second filler sequence may be located between the MCS region and 3′ ITR. In some embodiments, a viral genome may comprise two filler sequences, the first filler sequence may be located between the promoter region and enhancer region, and the second filler sequence may be located between the exon region and 3′ ITR.

In some embodiments, a viral genome may comprise two filler sequences, the first filler sequence may be located between the promoter region and polyadenylation signal sequence region, and the second filler sequence may be located between the payload region and intron region. In some embodiments, a viral genome may comprise two filler sequences, the first filler sequence may be located between the promoter region and polyadenylation signal sequence region, and the second filler sequence may be located between the payload region and enhancer region. In some embodiments, a viral genome may comprise two filler sequences, the first filler sequence may be located between the promoter region and polyadenylation signal sequence region, and the second filler sequence may be located between the payload region and polyadenylation signal sequence region. In some embodiments, a viral genome may comprise two filler sequences, the first filler sequence may be located between the promoter region and polyadenylation signal sequence region, and the second filler sequence may be located between the payload region and MCS region. In some embodiments, a viral genome may comprise two filler sequences, the first filler sequence may be located between the promoter region and polyadenylation signal sequence region, and the second filler sequence may be located between the payload region and exon region. In some embodiments, a viral genome may comprise two filler sequences, the first filler sequence may be located between the promoter region and polyadenylation signal sequence region, and the second filler sequence may be located between the payload region and 3′ ITR. In some embodiments, a viral genome may comprise two filler sequences, the first filler sequence may be located between the promoter region and polyadenylation signal sequence region, and the second filler sequence may be located between the intron region and enhancer region. In some embodiments, a viral genome may comprise two filler sequences, the first filler sequence may be located between the promoter region and polyadenylation signal sequence region, and the second filler sequence may be located between the intron region and polyadenylation signal sequence region. In some embodiments, a viral genome may comprise two filler sequences, the first filler sequence may be located between the promoter region and polyadenylation signal sequence region, and the second filler sequence may be located between the intron region and MCS region. In some embodiments, a viral genome may comprise two filler sequences, the first filler sequence may be located between the promoter region and polyadenylation signal sequence region, and the second filler sequence may he located between the intron region and exon region. In some embodiments, a viral genome may comprise two filler sequences, the first filler sequence may be located between the promoter region and polyadenylation signal sequence region, and the second filler sequence may be located between the intron region and 3′ ITR. In some embodiments, a viral genome may comprise two filler sequences, the first filler sequence may be located between the promoter region and polyadenylation signal sequence region, and the second filler sequence may be located between the enhancer region and polyadenylation signal sequence region. In some embodiments, a viral genome may comprise two filler sequences, the first filler sequence may be located between the promoter region and polyadenylation signal sequence region, and the second filler sequence may be located between the enhancer region and MCS region, in some embodiments, a viral genome may comprise two filler sequences, the first filler sequence may be located between the promoter region and polyadenylation signal sequence region, and the second filler sequence may be located between the enhancer region and exon region. In some embodiments, a viral genome may comprise two filler sequences, the first filler sequence may be located between the promoter region and polyadenylation signal sequence region, and the second filler sequence may be located between the enhancer region and 3′ ITR. In some embodiments, a viral genome may comprise two filler sequences, the first filler sequence may be located between the promoter region and polyadenylation signal sequence region, and the second filler sequence may be located between the polyadenylation signal sequence region and MCS region. In some embodiments, a viral genome may comprise two filler sequences, the first filler sequence may be located between the promoter region and polyadenylation signal sequence region, and the second filler sequence may be located between the polyadenylation signal sequence region and exon region. In some embodiments, a viral genome may comprise two filler sequences, the first filler sequence may be located between the promoter region and polyadenylation signal sequence region, and the second filler sequence may be located between the polyadenylation signal sequence region and 3′ ITR, in some embodiments, a viral genome may comprise two filler sequences, the first filler sequence may be located between the promoter region and polyadenylation signal sequence region, and the second filler sequence may be located between the MCS region and exon region. In some embodiments, a viral genome may comprise two filler sequences, the first filler sequence may be located between the promoter region and polyadenylation signal sequence region, and the second filler sequence may be located between the MCS region and 3′ ITR. In some embodiments, a viral genome may comprise two filler sequences, the first filler sequence may be located between the promoter region and polyadenylation signal sequence region, and the second filler sequence may he located between the exon region and 3′ ITR.

In some embodiments, a viral genome may comprise two filler sequences, the first filler sequence may be located between the promoter region and exon region, and the second filler sequence may be located between the payload region and intron region. In some embodiments, a viral genome may comprise two filler sequences, the first filler sequence may be located between the promoter region and exon region, and the second filler sequence may be located between the payload region and enhancer region. In some embodiments, a viral genome may comprise two filler sequences, the first filler sequence may be located between the promoter region and exon region, and the second filler sequence may be located between the payload region and polyadenylation signal sequence region. In some embodiments, a viral genome may comprise two filler sequences, the first filler sequence may be located between the promoter region and exon region, and the second filler sequence may be located between the payload region and MCS region. In some embodiments, a viral genome may comprise two filler sequences, the first filler sequence may be located between the promoter region and exon region, and the second filler sequence may be located between the payload region and exon region. In some embodiments, a viral genome may comprise two filler sequences, the first filler sequence may be located between the promoter region and exon region, and the second filler sequence may be located between the payload region and 3′ ITR. In some embodiments, a viral genome may comprise two filler sequences, the first filler sequence may be located between the promoter region and exon region, and the second filler sequence may be located between the intron region and enhancer region. In some embodiments, a viral genome may comprise two filler sequences, the first filler sequence may be located between the promoter region and exon region, and the second filler sequence may be located between the intron region and polyadenylation signal sequence region. In some embodiments, a viral genome may comprise two filler sequences, the first filler sequence may be located between the promoter region and exon region, and the second filler sequence may be located between the intron region and MCS region. In some embodiments, a viral genome may comprise two filler sequences, the first filler sequence may be located between the promoter region and exon region, and the second filler sequence may be located between the intron region and exon region. In some embodiments, a viral genome may comprise two filler sequences, the first filler sequence may be located between the promoter region and exon region, and the second filler sequence may be located between the intron region and 3′ ITR. In some embodiments, a viral genome may comprise two filler sequences, the first filler sequence may be located between the promoter region and exon region, and the second filler sequence may be located between the enhancer region and polyadenylation signal sequence region. In some embodiments, a viral genome may comprise two filler sequences, the first filler sequence may be located between the promoter region and exon region, and the second filler sequence may be located between the enhancer region and MCS region. In some embodiments, a viral genome may comprise two filler sequences, the first filler sequence may be located between the promoter region and exon region, and the second filler sequence may be located between the enhancer region and exon region. In some embodiments, a viral genome may comprise two filler sequences, the first filler sequence may be located between the promoter region and exon region, and the second filler sequence may be located between the enhancer region and 3′ ITR. In some embodiments, a viral genome may comprise two filler sequences, the first filler sequence may be located between the promoter region and exon region, and the second filler sequence may be located between the polyadenylation signal sequence region and MCS region. In some embodiments, a viral genome may comprise two filler sequences, the first filler sequence may be located between the promoter region and exon region, and the second filler sequence may be located between the polyadenylation signal sequence region and exon region. In some embodiments, a viral genome may comprise two filler sequences, the first fill sequence may be located between the promoter region and exon region, and the second filler sequence may be located between the polyadenylation signal sequence region and 3′ ITR. In some embodiments, a viral genome may comprise two filler sequences, the first filler sequence may be located between the promoter region and exon region, and the second filler sequence may be located between the MCS region and exon region. In some embodiments, a viral genome may comprise two filler sequences, the first filler sequence may be located between the promoter region and exon region, and the second filler sequence may be located between the MCS region and 3′ ITR, In some embodiments, a viral genome may comprise two filler sequences, the first filler sequence may be located between the promoter region and exon region, and the second filler sequence may be located between the exon region and 3′ ITR.

In some embodiments, a viral genome may comprise two filler sequences, the first filler sequence may be located between the promoter region and MCS region, and the second filler sequence may be located between the payload region and intron region. In some embodiments, a viral genome may comprise two filler sequences, the first filler sequence may be located between the promoter region and MCS region, and the second filler sequence may be located between the payload region and enhancer region. In some embodiments, a viral genome may comprise two filler sequences, the first filler sequence may be located between the promoter region and MCS region, and the second filler sequence may be located between the payload region and polyadenylation signal sequence region. In some embodiments, a viral genome may comprise two filler sequences, the first filler sequence may be located between the promoter region and MCS region, and the second filler sequence may be located between the payload region and MCS region. In some embodiments, a viral genome may comprise two filler sequences, the first filler sequence may be located between the promoter region and MCS region, and the second filler sequence may be located between the payload region and exon region. In some embodiments, a viral genome may comprise two filler sequences, the first filler sequence may be located between the promoter region and MCS region, and the second filler sequence may be located between the payload region and 3′ ITR. In some embodiments, a viral genome may comprise two filler sequences, the first filler sequence may be located between the promoter region and MCS region, and the second filler sequence may be located between the intron region and enhancer region. in some embodiments, a viral genome may comprise two filler sequences, the first filler sequence may be located between the promoter region and MCS region, and the second filler sequence may be located between the intron region and polyadenylation signal sequence region. In some embodiments, a viral genome may comprise two filler sequences, the first filler sequence may be located between the promoter region and MCS region, and the second filler sequence may be located between the intron region and MCS region. In some embodiments, a viral genome may comprise two filler sequences, the first filler sequence may be located between the promoter region and MCS region, and the second filler sequence may be located between the intron region and exon region. In some embodiments, a viral genome may comprise two filler sequences, the first filler sequence may be located between the promoter region and MCS region, and the second filler sequence may be located between the intron region and 3′ ITR. In some embodiments, a viral genome may comprise two filler sequences, the first filler sequence may be located between the promoter region and MCS region, and the second filler sequence may be located between the enhancer region and polyadenylation signal sequence region. In some embodiments, a viral genome may comprise two filler sequences, the first filler sequence may be located between the promoter region and MCS region, and the second filler sequence may be located between the enhancer region and MCS region. In some embodiments, a viral genome may comprise two filler sequences, the first filler sequence may be located between the promoter region and MCS region, and the second filler sequence may be located between the enhancer region and exon region. In some embodiments, a viral genome may comprise two filler sequences, the first filler sequence may be located between the promoter region and MCS region, and the second filler sequence may be located between the enhancer region and 3′ ITR, In some embodiments, a viral genome may comprise two filler sequences, the first filler sequence may be located between the promoter region and MCS region, and the second filler sequence may be located between the polyadenylation signal sequence region and MCS region. In some embodiments, a viral genome may comprise two filler sequences, the first filler sequence may be located between the promoter region and MCS region, and the second filler sequence may be located between the polyadenylation signal sequence region and exon region. In some embodiments, a viral genome may comprise two filler sequences, the first filler sequence may be located between the promoter region and MCS region, and the second filler sequence may be located between the polyadenylation signal sequence region and 3′ ITR, In some embodiments, a viral genome may comprise two filler sequences, the first filler sequence may be located between the promoter region and MCS region, and the second filler sequence may be located between the MCS region and exon region. In some embodiments, a viral genome may comprise two filler sequences, the first filler sequence may be located between the promoter region and MCS region, and the second filler sequence may be located between the MCS region and 3′ ITR. In some embodiments, a viral genome may comprise two filler sequences, the first filler sequence may be located between the promoter region and MCS region, and the second filler sequence may be located between the exon region and 3′ ITR.

In some embodiments, a viral genome may comprise two filler sequences, the first filler sequence may be located between the promoter region and 3′ ITR, and the second filler sequence may be located between the payload region and intron region. In some embodiments, a viral genome may comprise two filler sequences, the first filler sequence may be located between the promoter region and 3′ ITR, and the second filler sequence may be located between the payload region and enhancer region. In some embodiments, a viral genome may comprise two filler sequences, the first filler sequence may be located between the promoter region and 3′ ITR, and the second filler sequence may be located between the payload region and polyadenylation signal sequence region. In some embodiments, a viral genome may comprise two filler sequences, the first filler sequence may be located between the promoter region and 3′ ITR, and the second filler sequence may be located between the payload region and MCS region. In some embodiments, a viral genome may comprise two filler sequences, the first filler sequence may he located between the promoter region and 3′ ITR, and the second filler sequence may be located between the payload region and exon region. In some embodiments, a viral genome may comprise two filler sequences, the first filler sequence may be located between the promoter region and 3′ ITR, and the second filler sequence may be located between the payload region and 3′ ITR. In some embodiments, a viral genome may comprise two filler sequences, the first filler sequence may be located between the promoter region and 3′ ITR, and the second filler sequence may be located between the intron region and enhancer region. In some embodiments, a viral genome may comprise two filler sequences, the first filler sequence may be located between the promoter region and 3′ ITR, and the second filler sequence may be located between the intron region and polyadenylation signal sequence region. In some embodiments, a viral genome may comprise two filler sequences, the first filler sequence may be located between the promoter region and 3′ ITR, and the second filler sequence may be located between the intron region and MCS region. In some embodiments, a viral genome may comprise two filler sequences, the first filler sequence may be located between the promoter region and 3′ ITR, and the second filler sequence may be located between the intron region and exon region. In some embodiments, a viral genome may comprise two filler sequences, the first filler sequence may be located between the promoter region and 3′ ITR, and the second filler sequence may be located between the intron region and 3′ ITR In some embodiments, a viral genome may comprise two filler sequences, the first filler sequence may be located between the promoter region and 3′ ITR, and the second filler sequence may be located between the enhancer region and polyadenylation signal sequence region. In some embodiments, a viral genome may comprise two filler sequences, the first filler sequence may be located between the promoter region and 3′ ITR, and the second filler sequence may be located between the enhancer region and MCS region. In some embodiments, a viral genome may comprise two filler sequences, the first filler sequence may be located between the promoter region and 3′ ITR, and the second filler sequence may be located between the enhancer region and exon region. In some embodiments, a viral genome may comprise two filler sequences, the first filler sequence may be located between the promoter region and 3′ ITR, and the second filler sequence may be located between the enhancer region and 3′ ITR. In some embodiments, a viral genome may comprise two filler sequences, the first filler sequence may be located between the promoter region and 3′ ITR, and the second filler sequence may be located between the polyadenylation signal sequence region and MCS region. In some embodiments, a viral genome may comprise two filler sequences, the first filler sequence may be located between the promoter region and 3′ ITR, and the second filler sequence may be located between the polyadenylation signal sequence region and exon region. In some embodiments, a viral genome may comprise two filler sequences, the first filler sequence may be located between the promoter region and 3′ ITR, and the second filler sequence may be located between the polyadenylation signal sequence region and 3′ ITR. In some embodiments, a viral genome may comprise two filler sequences, the first filler sequence may be located between the promoter region and 3′ ITR, and the second filler sequence may be located between the MCS region and exon region. In some embodiments, a viral genome may comprise two filler sequences, the first filler sequence may be located between the promoter region and 3′ ITR, and the second filler sequence may be located between the MCS region and 3′ ITR. In some embodiments, a viral genome may comprise two filler sequences, the first filler sequence may be located between the promoter region and 3′ ITR, and the second filler sequence may be located between the exon region and 3′ ITR.

In some embodiments, a viral genome may comprise two filler sequences, the first filler sequence may be located between the payload region and intron region, and the second filler sequence may be located between the intron region and enhancer region. In some embodiments, a viral genome may comprise two filler sequences, the first filler sequence may be located between the payload region and intron region, and the second filler sequence may be located between the intron region and polyadenylation signal sequence region. In some embodiments, a viral genome may comprise two filler sequences, the first filler sequence may be located between the payload region and intron region, and the second filler sequence may be located between the intron region and MCS region. In some embodiments, a viral genome may comprise two filler sequences, the first filler sequence may be located between the payload region and intron region, and the second filler sequence may be located between the intron region and exon region. In some embodiments, a viral genome may comprise two filler sequences, the first filler sequence may be located between the payload region and intron region, and the second filler sequence may be located between the intron region and 3′ ITR. In some embodiments, a viral genome may comprise two filler sequences, the first filler sequence may he located between the payload region and intron region, and the second filler sequence may be located between the enhancer region and polyadenylation signal sequence region. In some embodiments, a viral genome may comprise two filler sequences, the first filler sequence may be located between the payload region and intron region, and the second filler sequence may be located between the enhancer region and MCS region. In some embodiments, a viral genome may comprise two filler sequences, the first filler sequence may be located between the payload region and intron region, and the second filler sequence may be located between the enhancer region and exon region. In some embodiments, a viral genome may comprise two filler sequences, the first filler sequence may be located between the payload region and intron region, and the second filler sequence may be located between the enhancer region and 3′ ITR. In some embodiments, a viral genome may comprise two filler sequences, the first filler sequence may be located between the payload region and intron region, and the second filler sequence may be located between the polyadenylation signal sequence region and MCS region. In some embodiments, a viral genome may comprise two filler sequences, the first filler sequence may be located between the payload region and intron region, and the second filler sequence may be located between the polyadenylation signal sequence region and exon region. In some embodiments, a viral genome may comprise two filler sequences, the first filler sequence may be located between the payload region and intron region, and the second filler sequence may be located between the polyadenylation signal sequence region and 3′ ITR. In some embodiments, a viral genome may comprise two filler sequences, the first filler sequence may be located between the payload region and intron region, and the second filler sequence may be located between the MCS region and exon region. In some embodiments, a viral genome may comprise two filler sequences, the first filler sequence may be located between the payload region and intron region, and the second filler sequence may be located between the MCS region and 3′ ITR. In some embodiments, a viral genome may comprise two filler sequences, the first filler sequence may be located between the payload region and intron region, and the second filler sequence may be located between the exon region and 3′ ITR.

In some embodiments, a viral genome may comprise two filler sequences, the first filler sequence may be located between the payload region and enhancer region, and the second filler sequence may be located between the intron region and enhancer region. In some embodiments, a viral genome may comprise two filler sequences, the first filler sequence may be located between the payload region and enhancer region, and the second filler sequence may be located between the intron region and polyadenylation signal sequence region. In some embodiments, a viral genome may comprise two filler sequences, the first filler sequence may he located between the payload region and enhancer region, and the second filler sequence may be located between the intron region and MCS region. In some embodiments, a viral genome may comprise two filler sequences, the first filler sequence may be located between the payload region and enhancer region, and the second filler sequence may be located between the intron region and exon region. In some embodiments, a viral genome may comprise two filler sequences, the first filler sequence may be located between the payload region and enhancer region, and the second filler sequence may be located between the intron region and 3′ ITR. In some embodiments, a viral genome may comprise two filler sequences, the first filler sequence may be located between the payload region and enhancer region, and the second filler sequence may be located between the enhancer region and polyadenylation signal sequence region. In some embodiments, a viral genome may comprise two filler sequences, the first filler sequence may be located between the payload region and enhancer region, and the second filler sequence may be located between the enhancer region and MCS region. In some embodiments, a viral genome may comprise two filler sequences, the first filler sequence may be located between the payload region and enhancer region, and the second filler sequence may be located between the enhancer region and exon region. In some embodiments, a viral genome may comprise two filler sequences, the first filler sequence may be located between the payload region and enhancer region, and the second filler sequence may be located between the enhancer region and 3′ ITR. In some embodiments, a viral genome may comprise two filler sequences, the first filler sequence may be located between the payload region and enhancer region, and the second filler sequence may be located between the polyadenylation signal sequence region and MCS region. In some embodiments, a viral genome may comprise two filler sequences, the first filler sequence may be located between the payload region and enhancer region, and the second filler sequence may be located between the polyadenylation signal sequence region and exon region. In some embodiments, a viral genome may comprise two filler sequences, the first filler sequence may be located between the payload region and enhancer region, and the second filler sequence may be located between the polyadenylation signal sequence region and 3′ ITR, In some embodiments, a viral genome may comprise two filler sequences, the first filler sequence may be located between the payload region and enhancer region, and the second filler sequence may be located between the MCS region and exon region. In some embodiments, a viral genome may comprise two filler sequences, the first filler sequence may be located between the payload region and enhancer region, and the second filler sequence may be located between the MCS region and 3′ ITR. In some embodiments, a viral genome may comprise two filler sequences, the first filler sequence may be located between the payload region and enhancer region, and the second filler sequence may be located between the exon region and 3′ ITR.

In some embodiments, a viral genome may comprise two filler sequences, the first filler sequence may be located between the payload region and polyadenylation signal sequence region, and the second filler sequence may be located between the intron region and enhancer region. In some embodiments, a viral genome may comprise two filler sequences, the first filler sequence may be located between the payload region and polyadenylation signal sequence region, and the second filler sequence may be located between the intron region and polyadenylation signal sequence region. In some embodiments, a viral genome may comprise two filler sequences, the first filler sequence may be located between the payload region and polyadenylation signal sequence region, and the second filler sequence may be located between the intron region and MCS region. In some embodiments, a viral genome may comprise two filler sequences, the first filler sequence may be located between the payload region and polyadenylation signal sequence region, and the second filler sequence may be located between the intron region and exon region. In some embodiments, a viral genome may comprise two filler sequences, the first filler sequence may be located between the payload region and polyadenylation signal sequence region, and the second filler sequence may be located between the intron region and 3′ ITR, In some embodiments, a viral genome may comprise two filler sequences, the first filler sequence may be located between the payload region and polyadenylation signal sequence region, and the second filler sequence may be located between the enhancer region and polyadenylation signal sequence region. In some embodiments, a viral genome may comprise two filler sequences, the first filler sequence may be located between the payload region and polyadenylation signal sequence region, and the second filler sequence may be located between the enhancer region and MCS region. In some embodiments, a viral genome may comprise two filler sequences, the first filler sequence may be located between the payload region and polyadenylation signal sequence region, and the second filler sequence may be located between the enhancer region and exon region. In some embodiments, a viral genome may comprise two filler sequences, the first filler sequence may be located between the payload region and polyadenylation signal sequence region, and the second filler sequence may be located between the enhancer region and 3′ ITR. In some embodiments, a viral genome may comprise two filler sequences, the first filler sequence may be located between the payload region and polyadenylation signal sequence region, and the second filler sequence may be located between the polyadenylation signal sequence region and MCS region. In some embodiments, a viral genome may comprise two filler sequences, the first filler sequence may be located between the payload region and polyadenylation signal sequence region, and the second filler sequence may be located between the polyadenylation signal sequence region and exon region. In some embodiments, a viral genome may comprise two filler sequences, the first filler sequence may be located between the payload region and polyadenylation signal sequence region, and the second filler sequence may be located between the polyadenylation signal sequence region and 3′ ITR. In some embodiments, a viral genome may comprise two filler sequences, the first filler sequence may be located between the payload region and polyadenylation signal sequence region, and the second filler sequence may be located between the MCS region and exon region. In some embodiments, a viral genome may comprise two filler sequences, the first filler sequence may be located between the payload region and polyadenylation signal sequence region, and the second filler sequence may be located between the MCS region and 3′ ITR. In some embodiments, a viral genome may comprise two filler sequences, the first filler sequence may be located between the payload region and polyadenylation signal sequence region, and the second filler sequence may be located between the exon region and 3′ ITR.

In some embodiments, a viral genome may comprise two filler sequences, the first filler sequence may be located between the payload region and MCS region, and the second filler sequence may be located between the intron region and enhancer region, In some embodiments, a viral genome may comprise two filler sequences, the first filler sequence may be located between the payload region and MCS region, and the second filler sequence may be located between the intron region and polyadenylation signal sequence region. In some embodiments, a viral genome may comprise two filler sequences, the first filler sequence may be located between the payload region and MCS region, and the second filler sequence may be located between the intron region and MCS region. In some embodiments, a viral genome may comprise two filler sequences, the first filler sequence may be located between the payload region and MCS region, and the second filler sequence may be located between the intron region and exon region. In some embodiments, a viral genome may comprise two filler sequences, the first filler sequence may be located between the payload region and MCS region, and the second filler sequence may be located between the intron region and 3′ ITR. In some embodiments, a viral genome may comprise two filler sequences, the first filler sequence may be located between the payload region and MCS region, and the second filler sequence may be located between the enhancer region and polyadenylation signal sequence region. In some embodiments, a viral genome may comprise two filler sequences, the first filler sequence may be located between the payload region and MCS region, and the second filler sequence may be located between the enhancer region and MCS region. In some embodiments, a viral genome may comprise two filler sequences, the first filler sequence may be located between the payload region and MCS region, and the second filler sequence may be located between the enhancer region and exon region. In some embodiments, a viral genome may comprise two filler sequences, the first filler sequence may be located between the payload region and MCS region, and the second filler sequence may be located between the enhancer region and 3′ ITR. In some embodiments, a viral genome may comprise two filler sequences, the first filler sequence may be located between the payload region and MCS region, and the second filler sequence may be located between the polyadenylation signal sequence region and MCS region. In some embodiments, a viral genome may comprise two filler sequences, the first filler sequence may be located between the payload region and MCS region, and the second filler sequence may be located between the polyadenylation signal sequence region and exon region. In some embodiments, a viral genome may comprise two filler sequences, the first filler sequence may be located between the payload region and MCS region, and the second filler sequence may be located between the polyadenylation signal sequence region and 3′ ITR. In some embodiments, a viral genome may comprise two filler sequences, the first filler sequence may be located between the payload region and MCS region, and the second filler sequence may be located between the MCS region and exon region. In some embodiments, a viral genome may comprise two filler sequences, the first filler sequence may be located between the payload region and MCS region, and the second filler sequence may be located between the MCS region and 3′ ITR. In some embodiments, a viral genome may comprise two filler sequences, the first filler sequence may be located between the payload region and MCS region, and the second filler sequence may be located between the exon region and 3′ ITR.

In some embodiments, a viral genome may comprise two filler sequences, the first filler sequence may be located between the payload region and exon region, and the second filler sequence may be located between the intron region and enhancer region. In some embodiments, a viral genome may comprise two filler sequences, the first filler sequence may be located between the payload region and exon region, and the second filler sequence may be located between the intron region and polyadenylation signal sequence region. In some embodiments, a viral genome may comprise two filler sequences, the first filler sequence may be located between the payload region and exon region, and the second filler sequence may be located between the intron region and MCS region. In some embodiments, a viral genome may comprise two filler sequences, the first filler sequence may be located between the payload region and exon region, and the second filler sequence may be located between the intron region and exon region, in some embodiments, a viral genome may comprise two filler sequences, the first filler sequence may be located between the payload region and exon region, and the second filler sequence may be located between the intron region and 3′ ITR. In some embodiments, a viral genome may comprise two filler sequences, the first filler sequence may be located between the payload region and exon region, and the second filler sequence may be located between the enhancer region and polyadenylation signal sequence region. In some embodiments, a viral genome may comprise two filler sequences, the first filler sequence may be located between the payload region and exon region, and the second filler sequence may be located between the enhancer region and MCS region. In some embodiments, a viral genome may comprise two filler sequences, the first filler sequence may be located between the payload region and exon region, and the second filler sequence may be located between the enhancer region and exon region. In some embodiments, a viral genome may comprise two filler sequences, the first filler sequence may be located between the payload region and exon region, and the second filler sequence may be located between the enhancer region and 3′ ITR. In some embodiments, a viral genome may comprise two filler sequences, the first filler sequence may be located between the payload region and exon region, and the second filler sequence may be located between the polyadenylation signal sequence region and MCS region. In some embodiments, a viral genome may comprise two filler sequences, the first filler sequence may be located between the payload region and exon region, and the second filler sequence may be located between the polyadenylation signal sequence region and exon region. In some embodiments, a viral genome may comprise two filler sequences, the first filler sequence may be located between the payload region and exon region, and the second filler sequence may be located between the polyadenylation signal sequence region and 3′ ITR. In some embodiments, a viral genome may comprise two filler sequences, the first filler sequence may be located between the payload region and exon region, and the second filler sequence may be located between the MCS region and exon region. in some embodiments, a viral genome may comprise two filler sequences, the first filler sequence may be located between the payload region and exon region, and the second filler sequence may be located between the MCS region and 3′ ITR. In some embodiments, a viral genome may comprise two filler sequences, the first filler sequence may be located between the payload region and exon region, and the second filler sequence may be located between the exon region and 3′ ITR,

In some embodiments, a viral genome may comprise two filler sequences, the first filler sequence may be located between the payload region and 3′ ITR region, and the second filler sequence may be located between the intron region and enhancer region. In some embodiments, a viral genome may comprise two filler sequences, the first filler sequence may he located between the payload region and 3′ ITR region, and the second filler sequence may be located between the intron region and polyadenylation signal sequence region. In some embodiments, a viral genome may comprise two filler sequences, the first filler sequence may be located between the payload region and 3′ ITR region, and the second filler sequence may be located between the intron region and MCS region. In some embodiments, a viral genome may comprise two filler sequences, the first filler sequence may be located between the payload region and 3′ ITR region, and the second filler sequence may be located between the intron region and exon region. In some embodiments, a viral genome may comprise two filler sequences, the first filler sequence may be located between the payload region and 3′ ITR region, and the second filler sequence may be located between the intron region and 3′ ITR. In some embodiments, a viral genome may comprise two filler sequences, the first filler sequence may be located between the payload region and 3′ ITR region, and the second filler sequence may be located between the enhancer region and polyadenylation signal sequence region. In some embodiments, a viral genome may comprise two filler sequences, the first filler sequence may be located between the payload region and 3′ ITR region, and the second filler sequence may be located between the enhancer region and MCS region. In some embodiments, a viral genome may comprise two filler sequences, the first filler sequence may be located between the payload region and 3′ ITR region, and the second filler sequence may be located between the enhancer region and exon region. In some embodiments, a viral genome may comprise two filler sequences, the first filler sequence may be located between the payload region and 3′ ITR region, and the second filler sequence may be located between the enhancer region and 3′ ITR. In some embodiments, a viral genome may comprise two filler sequences, the first filler sequence may be located between the payload region and 3′ ITR region, and the second filler sequence may be located between the polyadenylation signal sequence region and MCS region. In some embodiments, a viral genome may comprise two filler sequences, the first filler sequence may be located between the payload region and 3′ ITR region, and the second filler sequence may be located between the polyadenylation signal sequence region and exon region. In some embodiments, a viral genome may comprise two filler sequences, the first filler sequence may be located between the payload region and 3′ ITR region, and the second filler sequence may be located between the polyadenylation siiznal sequence region and 3′ ITR. In some embodiments, a viral genome may comprise two filler sequences, the first filler sequence may be located between the payload region and 3′ ITR region, and the second filler sequence may be located between the MCS region and exon region. In some embodiments, a viral genome may comprise two filler sequences, the first filler sequence may be located between the payload region and 3′ ITR region, and the second filler sequence may be located between the MCS region and 3′ ITR. In some embodiments, a viral genome may comprise two filler sequences, the first filler sequence may be located between the payload region and 3′ ITR region, and the second filler sequence may be located between the exon region and 3′ ITR.

In some embodiments, a viral genome may comprise two filler sequences, the first filler sequence may be located between the intron region and enhancer region, and the second filler sequence may be located between the enhancer region and polyadenylation signal sequence region. In some embodiments, a viral genome may comprise two filler sequences, the first filler sequence may be located between the intron region and enhancer region, and the second filler sequence may be located between the enhancer region and MCS region. In some embodiments, a viral genome may comprise two filler sequences, the first filler sequence may be located between the intron region and enhancer region, and the second filler sequence may be located between the enhancer region and exon region. In some embodiments, a viral genome may comprise two filler sequences, the first filler sequence may be located between the intron region and enhancer region, and the second filler sequence may be located between the enhancer region and 3′ ITR. In some embodiments, a viral genome may comprise two filler sequences, the first filler sequence may be located between the intron region and enhancer region, and the second filler sequence may be located between the polyadenylation signal sequence region and MCS region. In some embodiments, a viral genome may comprise two filler sequences, the first filler sequence may be located between the intron region and enhancer region, and the second filler sequence may be located between the polyadenylation signal sequence region and exon region. In some embodiments, a viral genome may comprise two filler sequences, the first filler sequence may be located between the intron region and enhancer region, and the second filler sequence may be located between the polyadenylation signal sequence region and 3′ ITR. In some embodiments, a viral genome may comprise two filler sequences, the first filler sequence may be located between the intron region and enhancer region, and the second filler sequence may be located between the MCS region and exon region. In some embodiments, a viral genome may comprise two filler sequences, the first filler sequence may be located between the intron region and enhancer region, and the second filler sequence may be located between the MCS region and 3′ ITR. In some embodiments, a viral genome may comprise two filler sequences, the first filler sequence may be located between the intron region and enhancer region, and the second filler sequence may be located between the exon region and 3′ ITR.

In some embodiments, a viral genome may comprise two filler sequences, the first filler sequence may be located between the intron region and polyadenylation signal sequence region, and the second filler sequence may be located between the enhancer region and polyadenylation signal sequence region. In some embodiments, a viral genome may comprise two filler sequences, the first filler sequence may be located between the intron region and polyadenylation signal sequence region, and the second filler sequence may be located between the enhancer region and MCS region. In some embodiments, a viral genome may comprise two filler sequences, the first filler sequence may be located between the intron region and polyadenylation signal sequence region, and the second filler sequence may be located between the enhancer region and exon region. In some embodiments, a viral genome may comprise two filler sequences, the first filler sequence may be located between the intron region and polyadenylation signal sequence region, and the second filler sequence may be located between the enhancer region and 3′ ITR, In some embodiments, a viral genome may comprise two filler sequences, the first filler sequence may be located between the intron region and polyadenylation signal sequence region, and the second filler sequence may be located between the polyadenylation signal sequence region and MCS region. In some embodiments, a viral genome may comprise two filler sequences, the first filler sequence may be located between the intron region and polyadenylation signal sequence region, and the second filler sequence may be located between the polyadenylation signal sequence region and exon region. In some embodiments, a viral genome may comprise two filler sequences, the first filler sequence may be located between the intron region and polyadenylation signal sequence region, and the second filler sequence may be located between the polyadenylation signal sequence region and 3′ ITR. In some embodiments, a viral genome may comprise two filler sequences, the first filler sequence may be located between the intron region and polyadenylation signal sequence region, and the second filler sequence may be located between the MCS region and exon region. In some embodiments, a viral genome may comprise two filler sequences, the first filler sequence may be located between the intron region and polyadenylation signal sequence region, and the second filler sequence may be located between the MCS region and 3′ ITR. In some embodiments, a viral genome may comprise two filler sequences, the first filler sequence may he located between the intron region and polyadenylation signal sequence region, and the second filler sequence may be located between the exon region and 3′ ITR.

In some embodiments, a viral genome may comprise two filler sequences, the first filler sequence may be located between the intron region and MCS region, and the second filler sequence may be located between the enhancer region and polyadenylation signal sequence region. In some embodiments, a viral genome may comprise two filler sequences, the first filler sequence may be located between the intron region and MCS region, and the second filler sequence may be located between the enhancer region and MCS region. In some embodiments, a viral genome may comprise two filler sequences, the first filler sequence may be located between the intron region and MCS region, and the second filler sequence may be located between the enhancer region and exon region. In some embodiments, a viral genome may comprise two filler sequences, the first filler sequence may be located between the intron region and MCS region, and the second filler sequence may be located between the enhancer region and 3′ ITR. In some embodiments, a viral genome may comprise two filler sequences, the first filler sequence may be located between the intron region and MCS region, and the second filler sequence may be located between the polyadenylation signal sequence region and MCS region. In some embodiments, a viral genome may comprise two filler sequences, the first filler sequence may be located between the intron region and MCS region, and the second filler sequence may be located between the polyadenylation signal sequence region and exon region. In some embodiments, a viral genome may comprise two filler sequences, the first filler sequence may be located between the intron region and MCS region, and the second filler sequence may be located between the polyadenylation signal sequence region and 3′ ITR, In some embodiments, a viral genome may comprise two filler sequences, the first filler sequence may be located between the intron region and MCS region, and the second filler sequence may he located between the MCS region and exon region. In some embodiments, a viral genome may comprise two filler sequences, the first filler sequence may be located between the intron region and MCS region, and the second filler sequence may be located between the MCS region and 3′ ITR, In some embodiments, a viral genome may comprise two filler sequences, the first filler sequence may be located between the intron region and MCS region, and the second filler sequence may be located between the exon region and 3′ ITR.

In some embodiments, a viral genome may comprise two filler sequences, the first filler sequence may be located between the intron region and exon region, and the second filler sequence may be located between the enhancer region and polyadenylation signal sequence region. In some embodiments, a viral genome may comprise two filler sequences, the first filler sequence may be located between the intron region and exon region, and the second filler sequence may be located between the enhancer region and MCS region. In some embodiments, a viral genome may comprise two filler sequences, the first filler sequence may be located between the intron region and exon region, and the second filler sequence may be located between the enhancer region and exon region. In some embodiments, a viral genome may comprise two filler sequences, the first filler sequence may be located between the intron region and exon region, and the second filler sequence may be located between the enhancer region and 3′ ITR. In some embodiments, a viral genome may comprise two filler sequences, the first filler sequence may be located between the intron region and exon region, and the second filler sequence may be located between the polyadenylation signal sequence region and MCS region. In some embodiments, a viral genome may comprise two filler sequences, the first filler sequence may be located between the intron region and exon region, and the second filler sequence may be located between the polyadenylation signal sequence region and exon region. In some embodiments, a viral genome may comprise two filler sequences, the first filler sequence may be located between the intron region and exon region, and the second filler sequence may be located between the polyadenylation signal sequence region and 3′ ITR. In some embodiments, a viral genome may comprise two filler sequences, the first filler sequence may be located between the intron region and exon region, and the second filler sequence may be located between the MCS region and exon region. In some embodiments, a viral genome may comprise two filler sequences, the first filler sequence may be located between the intron region and exon region, and the second filler sequence may be located between the MCS region and 3′ ITR, In some embodiments, a viral genome may comprise two filler sequences, the first filler sequence may be located between the intron region and exon region, and the second filler sequence may be located between the exon region and 3′ ITR.

In some embodiments, a viral genome may comprise two filler sequences, the first filler sequence may be located between the intron region and 3′ ITR, and the second filler sequence may be located between the enhancer region and polyadenylation signal sequence region. In some embodiments, a viral genome may comprise two filler sequences, the first filler sequence may be located between the intron region and 3′ ITR, and the second filler sequence may be located between the enhancer region and MCS region. In some embodiments, a viral genome may comprise two filler sequences, the first filler sequence may be located between the intron region and 3′ IFR, and the second filler sequence may be located between the enhancer region and exon region. In some embodiments, a viral genome may comprise two filler sequences, the first filler sequence may be located between the intron region and 3′ ITR, and the second filler sequence may be located between the enhancer region and 3′ ITR. In some embodiments, a viral genome may comprise two filler sequences, the first filler sequence may be located between the intron region and 3′ ITR, and the second filler sequence may be located between the polyadenylation signal sequence region and MCS region, In some embodiments, a viral genome may comprise two filler sequences, the first filler sequence may be located between the intron region and 3′ ITR, and the second filler sequence may be located between the polyadenylation signal sequence region and exon region. In some embodiments, a viral genome may comprise two filler sequences, the first filler sequence may be located between the intron region and 3′ ITR, and the second filler sequence may be located between the polyadenylation signal sequence region and 3′ ITR. In some embodiments, a viral genome may comprise two filler sequences, the first filler sequence may be located between the intron region and 3′ ITR, and the second filler sequence may be located between the MCS region and exon region. In some embodiments, a viral genome may comprise two filler sequences, the first filler sequence may be located between the intron region and 3′ ITR, and the second filler sequence may he located between the MCS region and 3′ ITR. In some embodiments, a viral genome may comprise two filler sequences, the first filler sequence may be located between the intron region and 3′ ITR, and the second filler sequence may be located between the exon region and 3′ ITR.

In some embodiments, a viral genome may comprise two filler sequences, the first filler sequence may be located between the enhancer region and polyadenylation signal sequence region, and the second filler sequence may be located between the polyadenylation signal sequence region and MCS region. In some embodiments, a viral genome may comprise two filler sequences, the first filler sequence may be located between the enhancer region and polyadenylation signal sequence region, and the second filler sequence may be located between the polyadenylation signal sequence region and exon region. In some embodiments, a viral genome may comprise two filler sequences, the first filler sequence may be located between the enhancer region and polyadenylation signal sequence region, and the second filler sequence may be located between the polyadenylation signal sequence region and 3′ ITR. In some embodiments, a viral genome may comprise two filler sequences, the first filler sequence may be located between the enhancer region and polyadenylation signal sequence region, and the second filler sequence may be located between the MCS region and exon region. In some embodiments, a viral genome may comprise two filler sequences, the first filler sequence may be located between the enhancer region and polyadenylation signal sequence region, and the second filler sequence may be located between the MCS region and 3′ ITR. In some embodiments, a viral genome may comprise two filler sequences, the first filler sequence may be located between the enhancer region and polyadenylation signal sequence region, and the second filler sequence may be located between the exon region and 3′ ITR.

In some embodiments, a viral genome may comprise two filler sequences, the first filler sequence may be located between the enhancer region and MCS region, and the second filler sequence may be located between the polyadenylation signal sequence region and MCS region. In some embodiments, a viral genome may comprise two filler sequences, the first filler sequence may be located between the enhancer region and MCS region, and the second filler sequence may be located between the polyadenylation signal sequence region and exon region. In some embodiments, a viral genome may comprise two filler sequences, the first filler sequence may be located between the enhancer region and MCS region, and the second filler sequence may be located between the polyadenylation signal sequence region and 3′ ITR. In some embodiments, a viral genome may comprise two filler sequences, the first filler sequence may be located between the enhancer region and MCS region, and the second filler sequence may be located between the MCS region and exon region. In some embodiments, a viral genome may comprise two filler sequences, the first filler sequence may be located between the enhancer region and MCS region, and the second filler sequence may be located between the MCS region and 3′ ITR, in some embodiments, a viral genome may comprise two filler sequences, the first filler sequence may be located between the enhancer region and MCS region, and the second filler sequence may be located between the exon region and 3′ ITR.

In some embodiments, a viral genome may comprise two filler sequences, the first filler sequence may be located between the enhancer region and exon region, and the second filler sequence may be located between the polyadenylation signal sequence region and MCS region. In some embodiments, a viral genome may comprise two filler sequences, the first filler sequence may be located between the enhancer region and exon region, and the second filler sequence may be located between the polyadenylation signal sequence region and exon region. In some embodiments, a viral genome may comprise two filler sequences, the first filler sequence may be located between the enhancer region and exon region, and the second filler sequence may be located between the polyadenylation signal sequence region and 3′ ITR, In some embodiments, a viral genome may comprise two filler sequences, the first filler sequence may be located between the enhancer region and exon region, and the second filler sequence may be located between the MCS region and exon region. In some embodiments, a viral genome may comprise two filler sequences, the first filler sequence may be located between the enhancer region and exon region, and the second filler sequence may be located between the MCS region and 3′ ITR. In some embodiments, a viral genome may comprise two filler sequences, the first filler sequence may be located between the enhancer region and exon region, and the second filler sequence may be located between the exon region and 3′ ITR.

In some embodiments, a viral genome may comprise two filler sequences, the first filler sequence may be located between the enhancer region and 3′ ITR, and the second filler sequence may be located between the polyadenylation signal sequence region and MCS region. In some embodiments, a viral genome may comprise two filler sequences, the first filler sequence may be located between the enhancer region and 3′ ITR, and the second filler sequence may be located between the polyadenylation signal sequence region and exon region. In some embodiments, a viral genome may comprise two filler sequences, the first filler sequence may be located between the enhancer region and 3′ ITR, and the second filler sequence may be located between the polyadenylation signal sequence region and 3′ ITR, In some embodiments, a viral genome may comprise two filler sequences, the first filler sequence may be located between the enhancer region and 3′ ITR, and the second filler sequence may be located between the MCS region and exon region. In some embodiments, a viral genome may comprise two filler sequences, the first filler sequence may be located between the enhancer region and 3′ ITR, and the second filler sequence may be located between the MCS region and 3′ ITR. In some embodiments, a viral genome may comprise two filler sequences, the first filler sequence may be located between the enhancer region and 3′ ITR, and the second filler sequence may be located between the exon region and 3′ ITR.

In some embodiments, a viral genome may comprise two filler sequences, the first filler sequence may be located between the polyadenylation signal sequence region and MCS region, and the second filler sequence may be located between the MCS region and exon region. In some embodiments, a viral genome may comprise two filler sequences, the first filler sequence may be located between the polyadenylation signal sequence region and MCS region, and the second filler sequence may be located between the MCS region and 3′ ITR. In some embodiments, a viral genome may comprise two filler sequences, the first filler sequence may be located between the polyadenylation signal sequence region and MCS region, and the second filler sequence may be located between the exon region and 3′ ITR.

In some embodiments, a viral genome may comprise two filler sequences, the first filler sequence may be located between the polyadenylation signal sequence region and exon region, and the second filler sequence may be located between the MCS region and exon region. In some embodiments, a viral genome may comprise two filler sequences, the first filler sequence may be located between the polyadenylation signal sequence region and exon region, and the second filler sequence may be located between the MCS region and 3′ ITR. In some embodiments, a viral genome may comprise two filler sequences, the first filler sequence may be located between the polyadenylation signal sequence region and exon region, and the second filler sequence may be located between the exon region and 3′ ITR.

In some embodiments, a viral genome may comprise two filler sequences, the first filler sequence may be located between the polyadenylation signal sequence region and 3′ ITR, and the second filler sequence may be located between the MCS region and exon region. In some embodiments, a viral genome may comprise two filler sequences, the first filler sequence may be located between the polyadenylation signal sequence region and and the second filler sequence may be located between the MCS region and 3′ ITR. In some embodiments, a viral genome may comprise two filler sequences, the first filler sequence may be located between the polyadenylation signal sequence region and 3′ ITR, and the second filler sequence may be located between the exon region and 3′ ITR.

In some embodiments, a viral genome may comprise two filler sequences, the first filler sequence may be located between the MCS region and exon region, and the second filler sequence may be located between the exon region and 3′ ITR.

### Payloads of the Disclosure

The AAV particles of the present disclosure comprise at least one payload region. As used herein, “payload” or “payload region” refers to one or more polynucleotides or polynucleotide regions encoded by or within a viral genome or an expression product of such polynucleotide or polynucleotide region, e.g., a transgene, a polynucleotide encoding a polypeptide or multi-polypeptide or a modulatory nucleic acid or regulatory nucleic acid. Payloads of the present disclosure typically encode modulatory polynucleotides or fragments or variants thereof.

The payload region may be constructed in such a way as to reflect a region similar to or mirroring the natural organization of an mRNA.

The payload region may comprise a combination of coding and non-coding nucleic acid sequences.

In some embodiments, the AAV payload region may encode a coding or non-coding RNA.

In some embodiments, the AAV particle comprises a viral genome with a payload region comprising nucleic acid sequences encoding a siRNA, miRNA or other RNAi agent. In such an embodiment, a viral genome encoding more than one polypeptide may be replicated and packaged into a viral particle. A target cell transduced with a viral particle may express the encoded siRNA, miRNA or other RNAi agent inside a single cell.

### Modulatory Polynucleotides

In some embodiments, modulatory polynucleotides, e.g., RNA or DNA molecules, may be used to treat neurodegenerative disease, in particular, Huntington's Disease (HD). As used herein, a “modulatory polynucleotide” is any nucleic acid sequence(s) which functions to modulate (either increase or decrease) the level or amount of a target gene, e.g., mRNA or protein levels.

In some embodiments, the modulatory polynucleotides may comprise at least one nucleic acid sequence encoding at least one siRNA molecule. The nucleic acids may, independently if there is more than one, encode 1, 2, 3, 4, 5, 6, 7, 8, 9, or more than 9 siRNA molecules.

In some embodiments, the molecular scaffold may be located downstream of a CMV promoter, fragment or variant thereof.

In some embodiments, the molecular scaffold may be located downstream of a CBA promoter, fragment or variant thereof.

In some embodiments, the molecular scaffold may be a natural pri-miRNA scaffold located downstream of a CMV promoter. As a non-limiting example, the natural pri-miRNA scaffold is derived from the human miR155 scaffold.

In some embodiments, the molecular scaffold may be a natural pri-miRNA scaffold located downstream of a CBA promoter.

In some embodiments, the selection of a molecular scaffold and modulatory polynucleotide is determined by a method of comparing modulatory polynucleotides in pri-miRNA (see e.g., the method described by Miniarikova et al. Design, Characterization, and Lead Selection of Therapeutic miRNAs Targeting Huntingtin for Development of Gene Therapy for Huntington's Disease. Molecular Therapy-Nucleic Acids (2016) 5, e297 and International Publication No. WO2016102664; the contents of each of which are herein incorporated by reference in their entireties). The modulatory polynucleotide may, but it not limited to, targeting exon 1, CAG repeats, SNP rs362331 in exon 50 and/or SNP rs362307 in exon 67. To evaluate the activities of the modulatory polynucleotides, the molecular scaffold used which may be used is a human pri-miRNA scaffold (e.g., miR155 scaffold) and the promoter may be CMV. The activity may be determined in vitro using HEK293T cells and a reporter (e.g., Luciferase). For exon 1 targeting, the modulatory polynucleotide is determined to be efficient at HTT knockdown if the knockdown is 80% or greater. For CAG targeting, the modulatory polynucleotide is determined to be efficient at HTT knockdown if the knockdown is at least 60%, For SNP targeting, the modulatory polynucleotide is determined to be efficient at HTT knockdown if the knockdown is at least 60%. For allele selectivity for CAG repeats or SNP targeting the modulatory polynucleotides may comprise at least 1 substitution in order to improve allele selectivity. As a non-limiting example, substitution may be a G or C replaced with a T or corresponding U and A or T/U replaced by a C.

In order to evaluate the optimal molecular scaffold for the modulatory polynucleotide, the modulatory polynucleotide is used in pri-miRNA scaffolds with a CAG promoter. The constructs are co-transfected with a reporter (e.g., luciferase reporter) at 50 ng. Constructs with greater than 80% knockdown at 50 ng co-transfection are considered efficient. In one aspect, the constructs with strong guide-strand activity are preferred. The molecular scaffolds can be processed in HEK293T cells by NGS to determine guide-passenger ratios, and processing variability.

To evaluate the molecular scaffolds and modulatory polynucleotides in vivo the molecular scaffolds comprising the modulatory polynucleotides are packaged in AAV (e.g., the serotype may be AAV5 (see e.g., the method and constructs described in WO2015060722, the contents of which are herein incorporated by reference in their entirety)) and administered to an in vivo model (e.g., Hu128/21 HD mouse) and the guide-passenger ratios, 5′ and 3′ end processing, reversal of guide and passenger strands, and knockdown can be determined in different areas of the model.

In some embodiments, the selection of a molecular scaffold and modulatory polynucleotide is determined by a method of comparing modulatory polynucleotides in natural pri-miRNA and synthetic pri-miRNA. The modulatory polynucleotide may, but it not limited to, targeting an exon other than exon 1. To evaluate the activities of the modulatory polynucleotides, the molecular scaffold is used with a CBA promoter. In one aspect, the activity may be determined in vitro using HEK293T cells, HeLa cell and a reporter (e.g., Luciferase) and knockdown efficient modulatory polynucleotides showed HTT knockdown of at least 80% in the cell tested. Additionally, the modulatory polynucleotides which are considered most efficient showed low to no significant passenger strand (p-strand) activity. In another aspect, the endogenous HTT knockdown efficacy is evaluated by transfection in vitro using, HEK293T cells, HeLa cell and a reporter. Efficient modulatory polynucleotides show greater than 50% endogenous HTT knockdown. In yet another aspect, the endogenous HTT knockdown efficacy is evaluated in different cell types (e.g., HEK293, HeLa, primary astrocytes, U251 astrocytes, SH-SY5Y neuron cells, FRhK-4 rhesus macaque (Macaca mulatta) kidney cells, and fibroblasts from HD patients) by infection (e.g., AAV2). Efficient modulatory polynucleotides show greater than 60% endogenous HTT knockdown.

To evaluate the molecular scaffolds and modulatory polynucleotides in vivo the molecular scaffolds comprising the modulatory polynucleotides are packaged in AAV and administered to an in vivo model (e.g., YAC128 HD mouse) and the guide-passenger ratios, 5′ and 3′ end processing, ratio of guide to passenger strands, and knockdown can be determined in different areas of the model (e.g., tissue regions). The molecular scaffolds can be processed from in vivo samples by NGS to determine guide-passenger ratios, and processing variability.

In some embodiments, the modulatory polynucleotide is designed using at least one of the following properties: loop variant, seed mismatch/bulge/wobble variant, stem mismatch, loop variant and vassal stem mismatch variant, seed mismatch and basal stem mismatch variant, stein mismatch and basal stem mismatch variant, seed wobble and basal stem wobble variant, or a stem sequence variant.

siRNA Molecules

The present disclosure relates to RNA interference (RNAi.) induced inhibition of gene expression for treating neurodegenerative disorders. Provided herein are siRNA duplexes or encoded dsRNA that target the HTT gene (referred to herein collectively as “siRNA molecules”). Such siRNA duplexes or encoded dsRNA can reduce or silence HTT gene expression in cells, for example, medium spiny neurons, cortical neurons and/or astrocytes, thereby, ameliorating symptoms of Huntington's Disease (HD).

RNAi (also known as post-transcriptional gene silencing (PTGS), quelling, or co-suppression) is a post-transcriptional gene silencing process in which RNA molecules, in a sequence specific manner, inhibit gene expression, typically by causing the destruction of specific mRNA molecules. The active components of RNAi are short/small double stranded RNAs (dsRNAs), called small interfering RNAs (siRNAs), that typically contain 15-30 nucleotides (e.g., 19 to 25, 19 to 24 or 19-21 nucleotides) and 2 nucleotide 3′ overhangs and that match the nucleic acid sequence of the target gene. These short RNA species may be naturally produced in vivo by Dicer-mediated cleavage of larger dsRNAs and they are functional in mammalian cells.

Naturally expressed small RNA molecules, known as microRNAs (miRNAs), elicit gene silencing by regulating the expression of mRNAs. The miRNAs containing RNA Induced Silencing Complex (RISC) targets mRNAs presenting a perfect sequence complementarity with nucleotides 2-7 in the 5′ region of the miRNA which is called the seed region, and other base pairs with its 3′ region. miRNA mediated down regulation of gene expression may be caused by cleavage of the target mRNAs, translational inhibition of the target mRNAs, or mRNA decay. miRNA targeting sequences are usually located in the 3′ ITR of the target mRNAs. A single miRNA may target more than 100 transcripts from various genes, and one mRNA may be targeted by different miRNAs.

siRNA duplexes or dsRNA targeting a specific mRNA may be designed and synthesized in vitro and introduced into cells for activating RNAi processes. Elbashir et al. demonstrated that 21-nucleotide siRNA duplexes (termed small interfering RNAs) were capable of effecting potent and specific gene knockdown without inducing immune response in mammalian cells (Elbashir SM et al., Nature, 2001, 411, 494-498). Since this initial report, post-transcriptional gene silencing by siRNAs quickly emerged as a powerful tool for genetic analysis in mammalian cells and has the potential to produce novel therapeutics.

RNAi molecules which were designed to target against a nucleic acid sequence that encodes poly-glutamine repeat proteins which cause poly-glutamine expansion diseases such as Huntington's Disease, are described in U.S. Pat. Nos. 9,169,483 and 9,181,544 and International Patent Publication No. WO2015179525, the content of each of which is herein incorporated by reference in their entirety. U.S. Pat. Nos. 9,169,483 and 9,181,544 and International Patent Publication No. WO2015179525 each provide isolated RNA duplexes comprising a first strand of RNA (e.g., 15 contiguous nucleotides) and second strand of RNA (e.g., complementary to at least 12 contiguous nucleotides of the first strand) where the RNA duplex is about 15 to 30 base pairs in length. The first strand of RNA and second strand of RNA may be operably linked by an RNA loop (˜4 to 50 nucleotides) to form a hairpin structure which may be inserted into an expression cassette. Non-limiting examples of loop portions include SEQ ID NO: 9-14 of U.S. Pat. No. 9,169,483, the content of which is herein incorporated by reference in its entirety. Non-limiting examples of strands of RNA which may be used, either full sequence or part of the sequence, to form RNA duplexes include SEQ ID NO: 1-8 of U.S. Pat. No. 9,169,483 and SEQ ID NO: 1-11, 33-59, 208-210, 213-215 and 218-221 of U.S. Pat. No. 9,181,544, the contents of each of which is herein incorporated by reference in its entirety. Non-limiting examples of RNAi molecules include SEQ ID NOs: 1-8 of U.S. Pat. No. 9,169,483, SEQ ID NOs: 1-11, 33-59, 208-210, 213-215 and 218-221 of U.S. Pat. No. 9,181,544 and SEQ ID NOs: 1, 6, 7, and 35-38 of international Patent Publication No. WO2015179525, the contents of each of which is herein incorporated by reference in their entirety.

In vitro synthetized siRNA molecules may be introduced into cells in order to activate RNAi. An exogenous siRNA duplex, when it is introduced into cells, similar to the endogenous dsRNAs, can be assembled to form the RNA Induced Silencing Complex (RISC), a multiunit complex that interacts with RNA sequences that are complementary to one of the two strands of the siRNA duplex (i.e., the antisense strand). During the process, the sense strand (or passenger strand) of the siRNA is lost from the complex, while the antisense strand (or guide strand) of the siRNA is matched with its complementary RNA. In particular, the targets of siRNA containing RISC complexes are mRNAs presenting a perfect sequence complementarity. Then, siRNA mediated gene silencing occurs by cleaving, releasing and degrading the target.

The siRNA duplex comprised of a sense strand homologous to the target mRNA and an antisense strand that is complementary to the target mRNA offers much more advantage in terms of efficiency for target RNA destruction compared to the use of the single strand (ss)-siRNAs (e.g. antisense strand RNA or antisense oligonucleotides). In many cases, it requires higher concentration of the ss-siRNA to achieve the effective gene silencing potency of the corresponding duplex.

Any of the foregoing molecules may be encoded by a viral genome.

Design and Sequences of siRNA Duplexes Targeting HTT Gene

The present disclosure provides small interfering RNA (siRNA) duplexes (and modulatory polynucleotides encoding them) that target HTT mRNA to interfere with HTT gene expression and/or HTT protein production.

The encoded siRNA duplex of the present disclosure contains an antisense strand and a sense strand hybridized together forming a duplex structure, wherein the antisense strand is complementary to the nucleic acid sequence of the targeted HTT gene, and wherein the sense strand is homologous to the nucleic acid sequence of the targeted HTT gene. In some aspects, the 5′ end of the antisense strand has a 5′ phosphate group and the 3′ end of the sense strand contains a 3′hydroxyl group. In. other aspects, there are none, one or 2 nucleotide overhangs at the 3′end of each strand.

Some guidelines for designing siRNAs have been proposed in the art. These guidelines generally recommend generating a 19-nucleotide duplexed region, symmetric 2-3 nucleotide 3′overhangs, 5′-phosphate and 3′-hydroxyl groups targeting a region in the gene to be silenced. Other rules that may govern siRNA sequence preference include, but are not limited to, A/U at the 5′ end of the antisense strand; (ii) G/C at the 5′ end of the sense strand; (iii) at least five A/U residues in the 5′ terminal one-third of the antisense strand; and (iv) the absence of any GC stretch of more than 9 nucleotides in length. In accordance with such consideration, together with the specific sequence of a target gene, highly effective siRNA molecules essential for suppressing mammalian target gene expression may be readily designed.

According to the present disclosure, siRNA molecules (e.g., siRNA duplexes or encoded dsRNA) that target the HTT gene are designed. Such siRNA molecules can specifically, suppress HTT gene expression and protein production. In some aspects, the siRNA molecules are designed and used to selectively “knock out” HTT gene variants in cells, i.e., mutated HTT transcripts that are identified in patients with HD disease. In some aspects, the siRNA molecules are designed and used to selectively “knock down” HTT gene variants in cells. In other aspects, the siRNA molecules are able to inhibit or suppress both the wild type and mutated HTT gene.

In some embodiments, an siRNA molecule of the present disclosure comprises a sense strand and a complementary antisense strand in which both strands are hybridized together to form a duplex structure. The antisense strand has sufficient complementarity to the HTT mRNA sequence to direct target-specific RNAi, i.e., the siRNA molecule has a sequence sufficient to trigger the destruction of the target mRNA by the RNAi machinery or process.

In some embodiments, an siRNA molecule of the present disclosure comprises a sense strand and a complementary antisense strand in which both strands are hybridized together to form a duplex structure and where the start site of the hybridization to the HTT mRNA is between nucleotide 100 and 7000 on the HTT mRNA sequence. As a non-limiting example, the start site may be between nucleotide 100-150, 150-200, 200-250, 250-300, 300-350, 350-400, 400-450, 450-500, 500-550, 550-600, 600-650, 650-700, 700-70, 750-800, 800-850, 850-900, 900-950, 950-1000, 1000-1050, 1050-1100, 1100-1150, 1150-1200, 1200-1250, 1250-1300, 1300-1350, 1350-1400, 1400-1450, 1450-1500, 1500-1550, 1550-1600, 1600-1650, 1650-1700, 1700-1750, 1750-1800, 1800-1850, 1850-1900, 1900-1950, 1950-2000, 2000-2050, 2050-2100, 2100-2150, 2150-2200, 2200-2250, 2250-2300, 2300-2350, 2350-2400, 2400-2450, 2450-2500, 2500-2550, 2550-2600, 2600-2650, 2650-2700, 2700-2750, 2750-2800, 2800-2850, 2850-2900, 2900-2950, 2950-3000, 3000-3050, 3050-3100, 3100-3150, 3150-3200, 3200-3250, 3250-3300, 3300-3350, 3350-3400, 3400-3450, 3450-3500, 3500-3550, 3550-3600, 3600-3650, 3650-3700, 3700-3750, 3750-3800, 3800-3850, 3850-3900, 3900-3950, 3950-4000, 4000-4050, 4050-4100, 4100-4150, 4150-4200, 4200-4250, 4250-4300, 4300-4350, 4350-4400, 4400-4450, 4450-4500, 4500-4550, 4550-4600, 4600-4650, 4650-4700, 4700-4750, 4750-4800, 4800-4850, 4850-4900, 4900-4950, 4950-5000, 5000-5050, 5050-5100, 5100-5150, 5150-5200, 5200-5250, 5250-5300, 5300-5350, 5350-5400, 5400-5450, 5450-5500, 5500-5550, 5550-5600, 5600-5650, 5650-5700, 5700-5750, 5750-5800, 5800-5850, 5850-5900, 5900-5950, 5950-6000, 6000-6050, 6050-6100, 6100-6150, 6150-6200, 6200-6250, 6250-6300, 6300-6350, 6350-6400, 6400-6450, 6450-6500, 6500-6550, 6550-6600, 6600-6650, 6650-6700, 6700-6750, 6750-6800, 6800-6850, 6850-6900, 6900-6950, 6950-7000, 7000-7050, 7050-7100, 7100-7150, 7150-7200, 7200-7250, 7250-7300, 7300-7350, 7350-7400, 7400-7450, 7450-7500, 7500-7550, 7550-7600, 7600-7650, 7650-7700, 7700-7750, 7750-7800, 7800-7850, 7850-7900, 7900-7950, 7950-8000, 8000-8050, 8050-8100, 8100-8150, 8150-8200, 8200-8250, 8250-8300, 8300-8350, 8350-8400, 8400-8450, 8450-8500, 8500-8550, 8550-8600, 8600-8650, 8650-8700, 8700-8750, 8750-8800, 8800-8850, 8850-8900, 8900-8950, 8950-9000, 9000-9050, 9050-9100, 9100-9150, 9150-9200, 9200-9250, 9250-9300, 9300-9350, 9350-9400, 9400-9450, 9450-9500, 9500-9550, 9550-9600, 9600-9650, 9650-9700, 9700-9750, 9750-9800, 9800-9850, 9850-9900, 9900-9950, 9950-10000, 10000-10050, 10050-10100, 10100-10150, 10150-10200, 10200-10250, 10250-10300, 10300-10350, 10350-10400, 10400-10450, 10450-10500, 10500-10550, 10550-10600, 10600-10650, 10650-10700, 10700-10750, 10750-10800, 10800-10850, 10850-10900, 10900-10950, 10950-11000, 11050-11100, 11100-11150, 11150-11200, 11200-11250, 11250-11300, 11300-11350, 11350-11400, 11400-11450, 11450-11500, 11500-11550, 11550-11600, 11600-11650, 11650-11700, 11700-11750, 11750-11800, 11800-11850, 11850-11900, 11900-11950, 11950-12000, 12000-12050, 12050-12100, 12100-12150, 12150-12200, 12200-12250, 12250-12300, 12300-12350, 12350-12400, 12400-12450, 12450-12500, 12500-12550, 12550-12600, 12600-12650, 12650-12700, 12700-12750, 12750-12800, 12800-12850, 12850-12900, 12900-12950, 12950-13000, 13050-13100, 13100-13150, 13150-13200, 13200-13250, 13250-13300, 13300-13350, 13350-13400, 13400-13450, and 13450-13500 on the HTT mRNA sequence. As yet another non-limiting example, the start site may be nucleotide 315, 316, 317, 318, 319, 320, 321, 322, 323, 324, 325, 326, 327, 328, 329, 330, 331, 332. 333, 334, 335, 336, 337, 338, 339. 340, 341, 342, 343, 344, 345, 346, 347, 348, 349, 350, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 715, 716, 717, 718, 719, 720, 721, 722, 723, 724, 725, 875, 876, 877, 878, 879, 880, 881, 882, 883, 884, 885, 886, 887, 888, 889. 890, 891, 892, 893, 894, 895, 896, 897, 898. 899, 900, 1375, 1376, 1377, 1378, 1379, 1380, 1381, 1382, 1383, 1384, 1385, 1386, 1387, 1388, 1389, 1390, 1391, 1392, 1393, 1394, 1395, 1396, 1397, 1398, 1399. 1400, 1401, 1402, 1403, 1404, 1405, 1406, 1407, 1408, 1409, 1410, 1411, 1412, 1413, 1414, 1415, 1416, 1417, 1418, 1419, 1420, 1421, 1422, 1423, 1424, 1425, 1426, 1427, 1428, 1429, 1430, 1431, 1432, 1433, 1434, 1435, 1436, 1437, 1438, 1439, 1440, 1441, 1442, 1443, 1444, 1445, 1446, 1447, 1448, 1449, 1450, 1660, 1661, 1662, 1663, 1664, 1665, 1666, 1667, 1668, 1669, 1670, 1671, 1672, 1673, 1674, 1675, 2050, 2051, 2052, 2053, 2054, 2055, 2056, 2057, 2058, 2059, 2060, 2061, 2062, 2063, 2064, 2065, 2066, 2067, 2068, 2069, 2070, 2071, 2072, 2073, 2074, 2075, 2076, 2077, 2078, 2079, 2080, 2081, 2082, 2083, 2084, 2085, 2086, 2087, 2088, 2089, 2090, 2091, 2092, 2093, 2094, 2095, 2096, 2097, 2098, 2099, 2100, 2580, 2581, 2582, 2583, 2584, 2585, 2586, 2587, 2588. 2589, 2590, 2591, 2592, 2593, 2594, 2595, 2596, 2597, 2598, 2599, 2600, 2601, 2602, 2603, 2604, 2605, 4525, 4526, 4527, 4528, 4529, 4530, 4531, 4532, 4533, 4534, 4535, 4536, 4537, 4538, 4539, 4540, 4541, 4542, 4543, 4544, 4545, 4546, 4547, 4548, 4549, 4550, 4575, 4576, 4577, 4578, 4579, 4580, 4581, 4582, 4583, 4584, 4585, 4586, 4587, 4588, 4589, 4590, 4591, 4592, 4593, 4594, 4595, 4596, 4597, 4598, 4599, 4600, 4850, 4851, 4852, 4853, 4854, 4855, 4856, 4857, 4858, 4859, 4860, 4861, 4862, 4863, 4864, 4865, 4866, 4867, 4868, 4869, 4870, 4871, 4872, 4873, 4874, 4875, 4876, 4877, 4878, 4879, 4880, 4881, 4882, 4883, 4884, 4885, 4886, 4887, 4888, 4889, 4890, 4891, 4892, 4893, 4894, 4895, 4896, 4897, 4898, 4899, 4900, 5460, 5461, 5462, 5463, 5464, 5465, 5466, 5467, 5468, 5469, 5470, 5471, 5472, 5473, 5474, 5475, 5476, 5477, 5478, 5479, 5480, 6175, 6176, 6177, 6178, 6179, 6180, 6181, 6182, 6183, 6184, 6185, 6186, 6187, 6188, 6189, 6190, 6191, 6192, 6193, 6194, 6195, 6196, 6197, 6198, 6199, 6200, 6315, 6316, 6317, 6318, 6319, 6320, 6321, 6322, 6323, 6324, 6325, 6326, 6327, 6328, 6329, 6330, 6331, 6332, 6333, 6334, 6335, 6336, 6337, 6338, 6339, 6340, 6341, 6342, 6343, 6344, 6345, 6600, 6601, 6602, 6603, 6604, 6605, 6606, 6607, 6608, 6609, 6610, 6611, 6612, 6613, 6614, 6615, 6725, 6726, 6727, 6728, 6729, 6730, 6731, 6732, 6733, 6734, 6735, 6736, 6737, 6738, 6739, 6740, 6741, 6742, 6743, 6744, 6745, 6746, 6747, 6748, 6749, 6750, 6751, 6752, 6753, 6754, 6755, 6756, 6757, 6758, 6759, 6760, 6761, 6762, 6763, 6764, 6765, 6766, 6767, 6768, 6769, 6770, 6771, 6772, 6773, 6774, 6775, 7655, 7656, 7657, 7658, 7659, 7660, 7661, 7662, 7663, 7664, 7665, 7666, 7667, 7668, 7669, 7670, 7671, 7672, 8510, 8511, 8512, 8513, 8514, 8515, 8516, 8715, 8716, 8717, 8718, 8719, 8720, 8721, 8722, 8723, 8724, 8725, 8726, 8727, 8728, 8729, 8730, 8731, 8732, 8733, 8734, 8735, 8736, 8737, 8738, 8739, 8740, 8741, 8742, 8743, 8744, 8745, 9250, 9251, 9252, 9253, 9254, 9255, 9256, 9257, 9258, 9259, 9260, 9261, 9262, 9263, 9264, 9265, 9266, 9267, 9268, 9269, 9270, 9480, 9481, 9482, 9483, 9484, 9485, 9486, 9487, 9488, 9489, 9490, 9491, 9492, 9493, 9494, 9495, 9496, 9497, 9498, 9499, 9500, 9575, 9576, 9577, 9578, 9579, 9580, 9581, 9582, 9583, 9584, 9585, 9586, 9587, 9588, 9589, 9590, 10525, 10526, 10527, 10528, 10529, 10530, 10531, 10532, 10533, 10534, 10535, 10536, 10537, 10538, 10539, 10540, 11545, 11546, 11547, 11548, 11549, 11550, 11551, 11552, 11553, 11554, 11555, 11556, 11557, 11558, 11559, 11560, 11875, 11876, 11877, 11878, 11879, 11880, 11881, 11882, 11883, 11884, 11885, 11886, 11887, 11888, 11889, 11890, 11891, 11892, 11893, 11894, 11895, 11896, 11897, 11898, 11899, 11900, 11915, 11916, 11917, 11918, 11919, 11920, 11921, 11922, 11923, 11924, 11925, 11926, 11927, 11928, 11929, 11930, 11931, 11932, 11933, 11934, 11935, 11936, 11937, 11938, 11939, 11940, 13375, 13376, 13377, 13378, 13379, 13380, 13381, 13382, 13383, 13384, 13385, 13386, 13387, 13388, 13389 and 13390 on the HTT mRNA sequence.

In some embodiments, the antisense strand and target mRNA sequences have 100% complementarity. The antisense strand may be complementary to any part of the target mRNA sequence.

In other embodiments, the antisense strand and target mRNA sequences comprise at least one mismatch. As a non-limiting example, the antisense strand and the target mRNA sequence have at least 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, 98%, or 99% or at least 20-30%, 20-40%, 20-50%, 20-60%, 20-70%, 20-80%, 20-90%, 20-95%, 20-99%, 30-40%, 30-50%, 30-60%, 30-70%, 30-80%, 30-90%, 30-95%, 30-99%, 40-50%, 40-60%, 40-70%, 40-80%, 40-90%, 40-95%, 40-99%, 50-60%, 50-70%, 50-80%, 50-90%, 50-95%, 50-99%, 60-70%, 60-80%, 60-90%, 60- 95%, 60-99%, 70-80%, 70-90%, 70-95%, 70-99%, 80-90%, 80-95%, 80-99%, 90-95%, 90-99% or 95-99% complementarity.

In some embodiments, an siRNA or dsRNA includes at least two sequences that are complementary to each other.

According to the present disclosure, the encoded siRNA molecule has a length from about 10-50 or more nucleotides, i.e., each strand comprising 10-50 nucleotides (or nucleotide analogs). Preferably, the siRNA molecule has a length from about 15-30, e.g., 15, 16, 17, 18, 19, 20, 21, 22, 23, 24, 25, 26, 27, 28, 29, or 30 nucleotides in each strand, wherein one of the strands is sufficiently complementarily to a target region. In some embodiments, each strand of the siRNA molecule has a length from about 19 to 25, 19 to 24 or 19 to 21 nucleotides. In some embodiments, at least one strand of the siRNA molecule is 19 nucleotides in length. In some embodiments, at least one strand of the siRNA molecule is 20 nucleotides in length. In some embodiments, at least one strand of the siRNA molecule is 21 nucleotides in length. In some embodiments, at least one strand of the siRNA molecule is 22 nucleotides in length. In some embodiments, at least one strand of the siRNA molecule is 23 nucleotides in length. In some embodiments, at least one strand of the siRNA molecule is 24 nucleotides in length. In some embodiments, at least one strand of the siRNA molecule is 25 nucleotides in length.

In some embodiments, the encoded siRNA molecules of the present disclosure can be synthetic RNA duplexes comprising about 19 nucleotides to about 25 nucleotides, and two overhanging nucleotides at the 3′-end. In some aspects, the siRNA molecules may be unmodified RNA molecules. In other aspects, the siRNA molecules may contain at least one modified nucleotide, such as base, sugar or backbone modifications.

In some embodiments, the encoded siRNA molecules of the present disclosure may comprise a nucleotide sequence such as, but not limited to, the antisense (guide) sequences in Table 2 or a fragment or variant thereof. As a non-limiting example, the antisense sequence used in the siRNA molecule of the present disclosure is at least 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, 98% or 99% or at least 20-30%, 20-40%, 20-50%, 20-60%, 20-70%, 20-80%, 20-90%, 20-95%, 20-99%, 30-40%, 30-50%, 30-60%, 30-70%, 30-80%, 30-90%, 30-95%, 30-99%, 40-50%, 40-60%, 40-70%, 40-80%, 40-90%, 40-95%, 40-99%, 50-60%, 50-70%, 50-80%, 50-90%, 50-95%, 50- 99%, 60-70%, 60-80%, 60-90%, 60-95%, 60-99%, 70-80%, 70-90%, 70-95%, 70-99%, 80-90%, 80-95%, 80-99%, 90-95%, 90-99% or 95-99% of a nucleotide sequence in Table 2, As another non-limiting example, the antisense sequence used in the siRNA molecule of the present disclosure comprises at least 3, 4, 5, 6, 7, 8, 9, 10, 11, 12, 13, 14, 15, 16, 17, 18, 19, 20, 21 or more than 21 consecutive nucleotides of a nucleotide sequence in Table 2. As yet another non-limiting example, the antisense sequence used in the siRNA molecule of the present disclosure comprises nucleotides 1 to 22, 1 to 21, 1 to 20, 1 to 19, 1 to 18, 1 to 17, 1 to 16, 1 to 15, 1to 14, 1 to 13, 1 to 12, 1 to 11, 1 to 10, 1 to 9, 1 to 8, 2 to 22, 2 to 21, 2 to 20, 2 to 19, 2 to 18, 2 to 17, 2 to 16, 2 to 15, 2 to 14, 2 to 13, 2 to 12, 2 to 11, 2 to 10, 2 to 9, 2 to 8, 3 to 22, 3 to 21, 3 to 20, 3 to 19, 3 to 18, 3 to 17, 3 to 16, 3 to 15, 3 to 14, 3 to 13, 3 to 12, 3 to 11, 3 to 10, 3 to 9, 3 to 8, 4 to 22, 4 to 21, 4 to 20, 4 to 19, 4 to 18, 4 to 17, 4 to 16, 4 to 15, 4 to 14, 4 to 13, 4 to 12, 4 to 11, 4 to 10, 4 to 9, 4 to 8, 5 to 22, 5 to 21, 5 to 20, 5 to 19, 5 to 18, 5 to 17, 5 to 16, 5 to 15, 5 to 14, 5 to 13, 5 to 12, 5 to 11, 5 to 10, 5 to 9, 5 to 8, 6 to 22, 6 to 21, 6 to 20, 6 to 19, 6 to 18, 6 to 17, 6 to 16, 6 to 15, 6 to 14, 6 to 13, 6 to 12, 6 to 11, 6 to 10, 7 to 22, 7 to 21, 7 to 20, 7 to 19, 7 to 18, 7 to 17, 7 to 16, 7 to 15, 7 to 14, 7 to 13, 7 to 12, 8 to 22, 8 to 21, 8 to 20, 8 to 19, 8 to 18, 8 to 17, 8 to 16, 8 to 15, 8 to 14, 8 to 13, 8 to 12, 9 to 22, 9 to 21, 9 to 20, 9 to 19, 9 to 18, 9 to 17, to 16, 9 to 15, 9 to 14, 10 to 22, 10 to 21, 10 to 20, 10 to 19, 10 to 18, 10 to 17, 10 to 16, 10 to 15, 10 to 14, 11 to 22, 11 to 21, 11 to 20, 11 to 19, 11 to 18, 11 to 17, 11 to 16, 11 to 15, 11 to 14, 12 to 22, 12 to 21, 12 to 20, 12 to 19, 12 to 18, 12 to 17, 12 to 16, 13 to 22, 13 to 21, 13 to 20, 13 to 19, 13 to 18, 13 to 17, 13 to 16, 14 to 22, 14 to 21, 14 to 20, 14 to 19, 14 to 18, 14 to 17, 15 to 22, 15 to 21, 15 to 20, 15 to 19, 15 to 18, 16 to 22, 16 to 21, 16 to 20, 17 to 22, 17 to 21, or 18 to 22 of the sequences in Table 2.

TABLE 2
Antisense Sequences

| Antisense | | SEQ |
|---|---|---|
| ID | Sequence | ID NO |
| A-2000 | UUAACGUCAGUUCAUAAACUU | 914 |
| A-2000dt | UUAACGUCAGUUCAUAAACdTdT | 915 |
| A-2001 | UGUCGGUACCGUCUAACACUU | 916 |
| A-2001dt | UGUCGGUACCGUCUAACACdTdT | 917 |
| A-2002 | UAAGCAUGGAGCUAGCAGGUU | 918 |
| A-2002dt | UAAGCAUGGAGCUAGCAGGdTdT | 919 |
| A-2003 | UACAACGAGACUGAAUUGCUU | 920 |
| A-2003dt | UACAACGAGACUGAAUUGCdTdT | 921 |
| A-2004 | UUCAGUUCAUAAACCUGGAUU | 922 |
| A-2004dt | UUCAGUUCAUAAACCUGGAdTdT | 923 |
| A-2005 | UAACGUCAGUUCAUAAACCUU | 924 |
| A-2005dt | UAACGUCAGUUCAUAAACCdTdT | 925 |
| A-2006 | UCCGGUCACAACAUUGUGGUU | 926 |
| A-2006dt | UCCGGUCACAACAUUGAGGdTdT | 927 |
| A-2007 | UUGCACGGUUCUUUGUGACUU | 928 |
| A-2007dt | UUGCACGGUUCUUUGUCAGdTdT | 929 |
| A-2008 | UUUUAUAACAAGAGGUUCAUU | 930 |
| A-2008dt | UUUUAUAACAAGAGGUUCAdTdT | 931 |
| A-2009 | UCCAAAUACUGGUUGUCGGUU | 932 |
| A-2009dt | UCCAAAUACUGGUUGUCGGdTdT | 933 |
| A-2010 | UAUUUUAGGAAUUCCAAUGUU | 934 |
| A-2010dt | UAUUUUAGGAAUUCCAAUGdTdT | 935 |
| A-2011 | UUUAGGAAUUCCAAUGAUCUU | 936 |
| A-2011dt | UUUAGGAAUUCCAAUGAUCdTdT | 937 |
| A-2012dt | UUAAUCUCUUUACUGAUAUdTdT | 938 |
| A-2013dt | GAUUUUAGGAAUUCCAAUGdTdT | 939 |
| A-2014 | UAAGCAUGGAGCUAGCAGGCUU | 940 |
| A-2015 | UAAGCAUGGAGCUAGCAGGGU | 941 |
| A-2016 | AAGGACUUGAGGGACUCGAAGU | 942 |
| A-2017 | AAGGACUUGAGGGACUCGAAG | 943 |
| A-2018 | AAGGACUUGAGGGACUCGA | 944 |
| A-2019 | AGGACUUGAGGGACUCGAAGU | 945 |
| A-2020 | GAGGACUUGAGGGACUCGAAGU | 946 |
| A-2021 | AAGGACUUGAGGGACUCGAAGU | 947 |
| A-2022 | AAGGACUUGAGGGACUCGAAGUU | 948 |
| A-2023 | AAGGACUUGAGGGACUCGAAG | 949 |
| A-2024 | AAGGACUUGAGGGACUCGA | 950 |
| A-2025 | AAGGACUUGAGGGACUCGAAGG | 951 |
| A-2026 | AAGGACUUGAGGGACUCGAAU | 952 |
| A-2027 | AAGGACUUGAGGGACUCGAAGA | 953 |
| A-2028 | AAGGACUUGAGGGACUCGAAGG | 954 |
| A-2029 | AAGGACUUGAGGGACUCGAAGGU | 955 |
| A-2030 | AAGGACUUGAGGGACUCGAAGGA | 956 |
| A-2031 | AAGGACUUGAGGGACUCGAAG | 957 |
| A-2032 | AAGGACUUGAGGGACUCGAAGU | 958 |
| A-2033 | AAGGACUUGAGGGACUCGA | 959 |
| A-2034 | AAGGACUUGAGGGACUCGAAGGA | 960 |
| A-2035 | AAGGACUUGAGGGACUCGAAGG | 961 |
| A-2036 | AAGGACUUGAGGGACUCGAAGGAU | 962 |
| A-2037 | AAGGACUUGAGGGACUCGAAGGAUU | 963 |
| A-2038 | AAGGACUUGAGGGACUCGAAG | 964 |
| A-2039 | AAGGACUUGAGGGACUCGAAGGAA | 965 |
| A-2040 | GAUGAAGUGCACACAUUGGAUGA | 966 |
| A-2041 | GAUGAACUGCACACAUUGGAUG | 967 |
| A-2042 | GAUGAAUUGCACACAGUAGAUGA | 968 |
| A-2043 | AAGGACUUGAGGGACUCGAAGGUU | 969 |
| A-2044 | AAGGACUUGAGGGACUCGAAGGUUU | 970 |
| A-2045 | AAGGACUUGAGGGACUCGAAGGU | 971 |
| A-2046 | AAGGACUUGAGGGACUCGAAGGUUUU | 972 |
| A-2047 | AAGGACUUGAGGGACUCGAAGGUUUUU | 973 |
| A-2048 | AAGGACUUGAGGGACUCGAAGG | 974 |
| A-2049 | UAAGGACUUGAGGGACUCGAAG | 975 |
| A-2050 | AAGGACUUGAGGGACUCGAAG | 976 |
| A-2051 | AAGGACUUGAGGGACUCGAAGU | 977 |
| A-2052 | AAGGACUUGAGGGACUCGAAGACGAGUCCC | 978 |
| A-2053 | AAGGACUUGAGGGACUCGAAGACGAGUCCCA | 979 |
| A-2054 | AAGGACUUGAGGGACUCGAAGACGAGUCCCU | 980 |
| A-2055 | GAUGAAGUGCACACAUUGGAUAC | 981 |
| A-2056 | GAUGAAGUGCACACAUUGGAUACA | 982 |
| A-2057 | GAUGAAGUGCACACAUUGGAUACAAUGUGU | 983 |
| A-2058 | GAUGAAGUGCACACAUUGGAU | 984 |
| A-2059 | GAUGAAGUGCACACAUUGGAUA | 985 |
| A-2060 | GAUGAAUUGCACACAGUAGAUAU | 986 |
| A-2061 | GAUGAAUUGCACACAGUAGAUAUAC | 987 |
| A-2062 | GAUGAAUUGCACACAGUAGAUAUACUGUGU | 988 |
| A-2063 | GAUGAAUUGCACACAGUAGAUAUA | 989 |
| A-2064 | AUGAAUUGCACACAGUAGAUAUAC | 990 |
| A-2065 | GAUGAAUUGCACACAGUAGAUA | 991 |
| A-2066 | GAUGAAUUGCACACAGUAGAUAUACUGUGU | 992 |
| A-2067 | UACAACGAGACUGAAUUGCU | 993 |
| A-2068 | ACAACGAGACUGAAUUGCUU | 994 |
| A-2069 | UCCGGUCACAACAUUGUGGUUC | 995 |
| A-2070 | UCCGGUCACAACAUUGUGGU | 996 |
| A-2071 | UCCGGUCACAACAUUGUG | 997 |
| A-2072 | CCGGUCACAACAUUGUGGUU | 998 |
| A-2073 | UUUUAUAACAAGAGGUUCAU | 999 |
| A-2074 | UUUAUAACAAGAGGUUCAUU | 1000 |
| A-2075 | UAAGCAUGGAGCUAGCAGGU | 1001 |
| A-2076 | AAGCAUGGAGCUAGCAGGUU | 1002 |
| A-2077 | CCAAAUACUGGUUGUCGGUU | 1003 |
| A-2078 | UACAACGAGACUGAAUUGCUUU | 1004 |
| A-2079 | UAACGUCAGUUCAUAAACCUUU | 1005 |
| A-2080 | GUCCGGUCACAACAUUGUGGUU | 1006 |
| A-2081 | UCCGGUCACAACAUUGUGGUUUG | 1007 |
| A-2082 | UCCGGUCACAACAUUGUGGUUU | 1008 |
| A-2083 | UCCGGUCACAACAUUGUGG | 1009 |
| A-2084 | UAAGCAUGGAGCUAGCAGGUUU | 1010 |
| A-2085 | AAGCAUGGAGCUAGCAGGUUU | 1011 |
| A-2086 | UCCAAAUACUGGUUGUCGGUUU | 1012 |
| A-2087 | CCAAAUACUGGUUGUCGGUUU | 1013 |

In some embodiments, the encoded siRNA molecules of the present disclosure may comprise a nucleotide sequence such as, but not limited to, the sense (passenger) sequences in Table 3 or a fragment or variant thereof. As a non-limiting example, the sense sequence used in the siRNA molecule of the present disclosure is at least 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, 98% or 99% or at least 20-30%, 20-40%, 20-50%, 20-60%, 20-70%, 20-80%, 20-90%, 20-95%, 20-99%, 30-40%, 30-50%, 30-60%, 30-70%, 30-80%, 30-90%, 30-95%, 30-99%, 40-50%, 40-60%, 40-70%, 40-80%, 40-90%, 40-95%, 40-99%, 50-60%, 50-70%, 50-80%, 50-90%, 50-95%, 50-99%, 60-70%, 60-80%, 60-90%, 60-95%, 60-99%, 70-80%, 70-90%, 70-95%, 70-99%, 80-90%, 80-95%, 80-99%, 90-95%, 90-99% or 95-99% of a nucleotide sequence in Table 3. As another non-limiting example, the sense sequence used in the siRNA molecule of the present disclosure comprises at least 3, 4, 5, 6, 7, 8, 9, 10, 11, 12, 13, 14, 15, 16, 17, 18, 19, 20, 21 or more than 21 consecutive nucleotides of a nucleotide sequence in Table 3. As yet another non-limiting example, the sense sequence used in the siRNA molecule of the present disclosure comprises nucleotides 1 to 22, 1 to 21, 1 to 20, 1 to 19, 1 to 18, 1to 17, 1to 16, 1 to 15, 1 to 14, 1 to 13, 1 to 12, 1 to 11, 1 to 10, 1 to 9, 1 to 8, 2 to 22, 2 to 21, 2 to 20, 2 to 19, 2 to 18, 2 to 17, 2 to 16, 2 to 15, 2 to 14, 2 to 13, 2 to 12, 2 to 11, 2 to 10, 2 to 9, 2 to 8, 3 to 22, 3 to 21, 3 to 20, 3 to 19, 3 to 18, 3 to 17, 3 to 16, 3 to 15, 3 to 14, 3 to 13, 3 to 12, 3 to 11, 3 to 10, 3 to 9, 3 to 8, 4 to 22, 4 to 21, 4 to 20, 4 to 19, 4 to 18, 4 to 17, 4 to 16, 4 to 15, 4 to 14, 4 to 13, 4 to 12, 4 to 11, 4 to 10, 4 to 9, 4 to 8, 5 to 22, 5 to 21, 5 to 20, 5 to 19, 5 to 18, 5 to 17, 5 to 16, 5 to 15, 5 to 14, 5 to 13, 5 to 12, 5 to 11, 5 to 10, 5 to 9, 5 to 8, 6 to 22, 6 to 21, 6 to 20, 6 to 19, 6 to 18, 6 to 17, 6 to 16, 6 to 15, 6 to 14, 6 to 13, 6 to 12, 6 to 11, 6 to 10, 7 to 22, 7 to 21, 7 to 20, 7 to 19, 7 to 18, 7 to 17, 7 to 16, 7 to 15, 7 to 14, 7 to 13, 7 to 12, 8 to 22, 8 to 21, 8 to 20, 8 to 19, 8 to 18, 8 to 17, 8 to 16, 8 to 15, 8 to 14, 8 to 13, 8 to 12, 9 to 22, 9 to 21, 9 to 20, 9 to 19, 9 to 18, 9 to 17, 9 to 16, 9 to 15, 9 to 14, 10 to 22, 10 to 21, 10 to 20, 10 to 19, 10 to 18, 10 to 17, 10 to 16, 10 to 15, 10 to 14, 11 to 22, 11 to 21, 11 to 20, 11 to 19, 11 to 18, 11 to 17, 11 to 16, 11 to 15, 11 to 14, 12 to 22, 12 to 21, 12 to 20, 12 to 19, 12 to 18, 12 to 17, 12 to 16, 13 to 22, 13 to 21, 13 to 20, 13 to 19, 13 to 18, 13 to 17, 13 to 16, 14 to 22, 14 to 21, 14 to 20, 14 to 19, 14 to 18, 14 to 17, 15 to 22, 15 to 21, 15 to 20, 15 to 19, 15 to 18, 16 to 22, 16 to 21, 16 to 20, 17 to 22, 17 to 21, or 18 to 22 of the sequences in Table 3.

TABLE 3
Sense Sequences

| | | SEQ |
|---|---|---|
| Sense ID | Sequence | ID NO |
| S-1000 | GUUUAUGAACUGAUCUUACCC | 1014 |
| S-1001 | GUGUUAGACGGUACUGAUCCC | 1015 |
| S-1002 | CCUGCUAGCUCCAUGCUUCCC | 1016 |
| S-1003 | GUUUAUGAACUGAUCUUAGCC | 1017 |
| S-1004 | GUGUUAGACGGUACUGAUGCC | 1018 |
| S-1005 | CCUGCUAGCUCCAUGCUUGCC | 1019 |
| S-1006 | GUUUAUGAAGUGAUCUUAACC | 1020 |
| S-1007 | GUGUUAGACCGUACUGAUACC | 1021 |
| S-1008 | CCUGCUAGCACCAUGCUUACC | 1022 |
| S-1009 | GUUUAUGAACUGAUCUUAACC | 1023 |
| S-1010 | GUGUUAGACGGUACUGAUACC | 1024 |
| S-1011 | CCUGCUAGCUCCAUGCUUACC | 1025 |
| S-1011dt | CCUGCUAGCUCCAUGCUUAdTdT | 1026 |
| S-1012 | GUUUAUGAACUGAUCUUGCCC | 1027 |
| S-1013 | GUUUAUGAACUGAUCUUGGCC | 1028 |
| S-1014 | GUUUAUGAACUGAUCUUGACC | 1029 |
| S-1015 | GCAAUUCAGUCUCGUUGUCCC | 1030 |
| S-1016 | UCCAGGUUUAUGAACUGACCC | 1031 |
| S-1017 | GGUUUAUGAACUGACGUUCCC | 1032 |
| S-1018 | CCACAAUGUUGUGACUGGCCC | 1033 |
| S-1019 | GUCACAAAGAACCGUGUACCC | 1034 |
| S-1020 | UGAACCUCUUGUUAUAAACCC | 1035 |
| S-1021 | CCGACAACCAGUAUUUGGCCC | 1036 |
| S-1022 | GCAAUUCAGUCUCGUUGUGCC | 1037 |
| S-1023 | UCCAGGUUUAUGAACUGAGCC | 1038 |
| S-1024 | GGUUUAUGAACUGACGUUGCC | 1039 |
| S-1025 | CCACAAUGUUGUGACUGGGCC | 1040 |
| S-1026 | GUCACAAAGAACCGUGUAGCC | 1041 |
| S-1027 | UGAACCUCUUGUUAUAAAGCC | 1042 |
| S-1028 | CCGACAACCAGUAUUUGGGCC | 1043 |
| S-1029 | GCAAUUCAGUCUCGUUGUACC | 1044 |
| S-1029dt | GCAAUUCAGUCUCGUUGUAdTdT | 1045 |
| S-1030 | UCCAGGUUUAUGAACUGAACC | 1046 |
| S-1030dt | UCCAGGUUUAUGAACUGAAdTdT | 1047 |
| S-1031 | GGUUUAUGAACUGACGUUACC | 1048 |
| S-1032 | CCACAAUGUUGUGACUGGACC | 1049 |
| S-1033 | GUCACAAAGAACCGUGUAACC | 1050 |
| S-1034 | UGAACCUCUUGUUAUAAAACC | 1051 |
| S-1034dt | UGAACCUCUUGUUAUAAAAdTdT | 1052 |
| S-1035 | CCGACAACCAGUAUUUGGACC | 1053 |
| S-1035dt | CCGACAACCAGUAUUUGGAdTdT | 1054 |
| S-1036 | GCAAUUCAGACUCGUUGUACC | 1055 |
| S-1037 | UCCAGGUUUUUGAACUGAACC | 1056 |
| S-1038 | GGUUUAUGAUCUGACGUUACC | 1057 |
| S-1039 | CCACAAUGUAGUGACUGGACC | 1058 |
| S-1040 | GUCACAAAGUACCGUGUAACC | 1059 |
| S-1041 | UGAACCUCUAGUUAUAAAACC | 1060 |
| S-1042 | CCGACAACCUGUAUUUGGACC | 1061 |
| S-1043 | CAUUGGAAUUCCUAAAAUUCC | 1062 |
| S-1044 | GAUCAUUGGAAUUCCUAAUCC | 1063 |
| S-1045 | CAUUGGAAUUCCUAAAAUGCC | 1064 |
| S-1046 | GAUCAUUGGAAUUCCUAAGCC | 1065 |
| S-1047 | CAUUGGAAUUCCUAAAAUACC | 1066 |
| S-1047dt | CAUUGGAAUUCCUAAAAUAdTdT | 1067 |
| S-1048 | GAUCAUUGGAAUUCCUAAACC | 1068 |
| S-1048dt | GAUCAUUGGAAUUCCUAAAdTdT | 1069 |
| S-1049 | CAUUGGAAUACCUAAAAUACC | 1070 |
| S-1050 | GAUCAUUGGUAUUCCUAAACC | 1071 |
| S-1051dt | GUUUAUGAACUGACGUUAAdTdT | 1072 |
| S-1052dt | GUGUUAGACGGUACCGACAdTdT | 1073 |
| S-1053dt | AUAUCAGUAAAGAGAUUAAdTdT | 1074 |
| S-1054dt | GGUUUAUGAACUGACGUUAdTdT | 1075 |
| S-1055dt | CCACAAUGUUGUGACCGGAdTdT | 1076 |
| S-1056dt | GACACAAAGAACCGUGCAAdTdT | 1077 |
| S-1057dt | CAUUGGAAUUCCUAAAAUCdTdT | 1078 |
| S-1058 | CCUGCUAGCUCCAUGCUUGCU | 1079 |
| S-1059 | CCUGCUAGCUCCAUGCUUGAU | 1080 |
| S-1060 | CCUGCUAGCUCCAUGCUUAUU | 1081 |
| S-1061 | CCUGCUAGCUCCAUGCUUGUU | 1082 |
| S-1062 | UUCGAGUCCCUCAAGUAGCU | 1083 |
| S-1063 | UUCGAGUCCCUCAAGUAGCUUU | 1084 |
| S-1064 | UCGAGUCCCUCAAGUCCAUUCU | 1085 |
| S-1065 | UUCCAGUCCAUCAAGUCAAUU | 1086 |
| S-1066 | UUCCGAGUCUAAAAGUCCUUGG | 1087 |
| S-1067 | UUCCGAGUCUAAAAGUCCUUGGC | 1088 |
| S-1068 | CUUCCGAGUCUAAAAGUCCUUGG | 1089 |
| S-1069 | UUCCGAGUCUAAAAGUCCUUGGU | 1090 |
| S-1070 | UUCCGAGUCUAAAAGUCCUUGGCU | 1091 |
| S-1071 | UCCAAUGUGAAACUUCAUCGGCU | 1092 |
| S-1072 | UCCAAUGUGAAACUUCAUCGGC | 1093 |
| S-1073 | AUCCAAUGUGAAACUUCAUCGU | 1094 |
| S-1074 | AUCCAAUGUGAAACUUCAUCGGU | 1095 |
| S-1075 | UCCAAUGUGAAACUUCAUCGGU | 1096 |
| S-1076 | UCCAUGUGAAACUUCAUCGGCUU | 1097 |
| S-1077 | AUCUACUGUGAAAAUUCAUCGG | 1098 |
| S-1078 | UCUACUGUGAAAAUUCAUCGG | 1099 |
| S-1079 | UCUACUGUGAAAAUUCAUCGGC | 1100 |
| S-1080 | AUCUACUGUGAAAAUUCAUCGGU | 1101 |
| S-1081 | UCUACUGUGAAAAUUCAUCGGU | 1102 |
| S-1082 | UCUACUGUGAAAAUUCAUCGGCU | 1103 |
| S-1083 | CCUUCGGUCCUCAAGUCCUUCA | 1104 |
| S-1084 | UUCGAGUCCAUCAAAUCCUAUAGU | 1105 |
| S-1085 | UACAAUGUGUGCACUUCAUAU | 1106 |
| S-1086 | UAUACUGUGUGCAAUUCAUUUCU | 1107 |
| S-1087 | GCAAUUCAGUCUCGUUGUCC | 1108 |
| S-1088 | GCAAUUCAGUCUCGUUGUC | 1109 |
| S-1089 | CAAUUCAGUCUCGUUGUCCC | 1110 |
| S-1090 | CAAUUCAGUCUCGUUGUCC | 1111 |
| S-1091 | GCAAUUCAGUCUCGUUGUGC | 1112 |
| S-1092 | CAAUUCAGUCUCGUUGUGCC | 1113 |
| S-1093 | CCACAAUGUUGUGACUGGGCCU | 1114 |
| S-1094 | CCACAAUGUUGUGACUGGGC | 1115 |
| S-1095 | CACAAUGUUGUGACUGGGCC | 1116 |
| S-1096 | UGAACCUCUUGUUAUAAAGCCU | 1117 |
| S-1097 | UGAACCUCUUGUUAUAAAGC | 1118 |
| S-1098 | GAACCUCUUGUUAUAAAGCC | 1119 |
| S-1099 | CCUGCUAGCUCCAUGCUUGCCU | 1120 |
| S-1100 | CCUGCUAGCUCCAUGCUUGC | 1121 |
| S-1101 | CCUGCUAGCUCCAUGCUUG | 1122 |
| S-1102 | CUGCUAGCUCCAUGCUUGCC | 1123 |
| S-1103 | CCGACAACCAGUAUUUGGGCCU | 1124 |
| S-1104 | CCGACAACCAGUAUUUGGGC | 1125 |
| S-1105 | CCGACAACCAGUAUUUGGG | 1126 |
| S-1106 | CGACAACCAGUAUUUGGGCC | 1127 |
| S-1107 | CGACAACCAGUAUUUGGGC | 1128 |
| S-1108 | GCAAUUCAGUCUCGUUGUACCU | 1129 |
| S-1109 | GCAAUUCAGUCUCGUUGUAC | 1130 |
| S-1110 | GCAAUUCAGUCUCGUUGUA | 1131 |
| S-1111 | CAAUUCAGUCUCGUUGUACC | 1132 |
| S-1112 | GCAAUUCAGACUCGUUGUACCU | 1133 |
| S-1113 | GCAAUUCAGACUCGUUGUAC | 1134 |
| S-1114 | GCAAUUCAGACUCGUUGUA | 1135 |
| S-1115 | CAAUUCAGACUCGUUGUACC | 1136 |
| S-1116 | AGCAAUUCAGUCUCGUUGUACC | 1137 |
| S-1117 | AGCAAUUCAGUCUCGUUGUAC | 1138 |
| S-1118 | AGGUUUAUGAACUGACGUUAC | 1139 |
| S-1119 | AGGUUUAUGAACUGACGUUACC | 1140 |
| S-1120 | ACCACAAUGUUGUGACUGGAC | 1141 |
| S-1121 | ACCACAAUGUUGUGACUGGACC | 1142 |
| S-1122 | CCACAAUGUUGUGACUGGACCGU | 1143 |
| S-1123 | CCACAAUGUUGUGACUGGACCG | 1144 |
| S-1124 | CCACAAUGUUGUGACUGGAC | 1145 |
| S-1125 | CACAAUGUUGUGACUGGACC | 1146 |
| S-1126 | ACCUGCUAGCUCCAUGCUUCCC | 1147 |
| S-1127 | ACCUGCUAGCUCCAUGCUUCC | 1148 |
| S-1128 | ACCUGCUAGCUCCAUGCUUC | 1149 |
| S-1129 | CCUGCUAGCUCCAUGCUUCC | 1150 |
| S-1130 | CCUGCUAGCUCCAUGCUUC | 1151 |
| S-1131 | CUGCUAGCUCCAUGCUUCCC | 1152 |
| S-1132 | CUGCUAGCUCCAUGCUUCC | 1153 |
| S-1133 | ACCGACAACCAGUAUUUGGACC | 1154 |
| S-1134 | ACCGACAACCAGUAUUUGGAC | 1155 |
| S-1135 | CCGACAACCAGUAUUUGGACCGU | 1156 |
| S-1136 | CCGACAACCAGUAUUUGGACCGU | 1157 |
| S-1137 | CCGACAACCAGUAUUUGGAC | 1158 |
| S-1138 | CGACAACCAGUAUUUGGACC | 1159 |
| S-1139 | CCUGCUAGCACCGUGCUUACC | 1160 |

In some embodiments, the siRNA molecules of the present disclosure may comprise an antisense sequence from Table 2 and a sense sequence from Table 3, or a fragment or variant thereof. As a non-limiting example, the antisense sequence and the sense sequence have at least 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, 98% or 99% or at least 20-30%, 20-40%, 20-50%, 20-60%, 20-70%, 20-80%, 20-90%, 20-95%, 20-99%, 30-40%, 30-50%, 30-60%, 30-70%, 30-80%, 30-90%, 30-95%, 30-99%, 40-50%, 40-60%, 40-70%, 40-80%, 40-90%, 40-95%, 40-99%, 50-60%, 50- 70%, 50-80%, 50-90%, 50-95%, 50-99%, 60-70%, 60-80%, 60-90%, 60-95%, 60-99%, 70-80%, 70-90%, 70-95%, 70-99%, 80-90%, 80-95%, 80-99%, 90-95%, 90-99% or 95-99% complementarity.

In some embodiments, the siRNA molecules of the present disclosure may comprise the sense and antisense siRNA duplex as described in Tables 4-6. As a non-limiting example, these siRNA duplexes may be tested for in vitro inhibitory activity on endogenous HTT gene expression. The start site for the sense and antisense sequence is compared to HTT gene sequence known as NM_002111.7 (SEQ ID NO: 1425) from NCBI.

TABLE 4
Sense and antisense strand sequences of HTT dsRNA

| | | | Sense | | | | Antisense | |
|---|---|---|---|---|---|---|---|---|
| siRNA | | | Strand | SS | | | Strand | AS |
| Duplex | | Start | Sequence | SEQ | | Start | Sequence | SEQ |
| ID | SS ID | SS | (5′-3′) | ID | AS ID | AS | (5′-3′) | ID |
| D-3566 | S-1058 | 6751 | CCUGCUAGCUC | 1079 | A-2002 | 6751 | UAAGCAUGGAG | 918 |
| | | | CAUGCUUGCU | | | | CUAGCAGGUU | |
| D-3567 | S-1058 | 6751 | CCUGCUAGCUC | 1079 | A-2014 | 6748 | UAAGCAUGGAG | 940 |
| | | | CAUGCUUGCU | | | | CUAGCAGGCUU | |
| D-3568 | S-1059 | 6751 | CCUGCUAGCUC | 1080 | A-2002 | 6751 | UAAGCAUGGAG | 918 |
| | | | CAUGCUUGAU | | | | CUAGCAGGUU | |
| D-3569 | S-1060 | 6751 | CCUGCUAGCUC | 1081 | A-2015 | 6751 | UAAGCAUGGAG | 941 |
| | | | CAUGCUUAUU | | | | CUAGCAGGGU | |
| D-3570 | S-1061 | 6751 | CCUGCUAGCUC | 1082 | A-2002 | 6751 | UAAGCAUGGAG | 918 |
| | | | CAUGCUUGUU | | | | CUAGCAGGUU | |
| D-3500 | S-1016 | 1386 | UCCAGGUUUAU | 1031 | A-2004 | 1386 | UUCAGUUCAUA | 922 |
| | | | GAACUGACCC | | | | AACCUGGAUU | |
| D-3501 | S-1023 | 1386 | UCCAGGUUUAU | 1038 | A-2004 | 1386 | UUCAGUUCAUA | 922 |
| | | | GAACUGAGCC | | | | AACCUGGAUU | |
| D-3502 | S-1030 | 1386 | UCCAGGUUUAU | 1046 | A-2004 | 1386 | UUCAGUUCAUA | 922 |
| | | | GAACUGAACC | | | | AACCUGGAUU | |
| D-3503 | S-1037 | 1386 | UCCAGGUUUUU | 1056 | A-2004 | 1386 | UUCAGUUCAUA | 922 |
| | | | GAACUGAACC | | | | AACCUGGAUU | |
| D-3504 | S-1030 | 1386 | UCCAGGUUUAU | 1046 | A-2001 | 2066 | UGUCGGUACCG | 916 |
| | | | GAACUGAACC | | | | UCUAACACUU | |
| D-3505 | S-1017 | 1390 | GGUUUAUGAAC | 1032 | A-2005 | 1389 | UAACGUCAGUU | 924 |
| | | | UGACGUUCCC | | | | CAUAAACCUU | |
| D-3506 | S-1024 | 1390 | GGUUUAUGAAC | 1039 | A-2005 | 1389 | UAACGUCAGUU | 924 |
| | | | UGACGUUGCC | | | | CAUAAACCUU | |
| D-3507 | S-1031 | 1390 | GGUUUAUGAAC | 1048 | A-2005 | 1389 | UAACGUCAGUU | 924 |
| | | | UGACGUUACC | | | | CAUAAACCUU | |
| D-3508 | S-1038 | 1390 | GGUUUAUGAUC | 1057 | A-2005 | 1389 | UAACGUCAGUU | 924 |
| | | | UGACGUUACC | | | | CAUAAACCUU | |
| D-3509 | S-1000 | 1391 | GUUUAUGAACU | 1014 | A-2000 | 1391 | UUAACGUCAGU | 914 |
| | | | GAUCUUACCC | | | | UCAUAAACUU | |
| D-3510 | S-1003 | 1391 | GUUUAUGAACU | 1017 | A-2000 | 1391 | UUAACGUCAGU | 914 |
| | | | GAUCUUAGCC | | | | UCAUAAACUU | |
| D-3511 | S-1006 | 1391 | GUUUAUGAAGU | 1020 | A-2000 | 1391 | UUAACGUCAGU | 914 |
| | | | GAUCUUAACC | | | | UCAUAAACUU | |
| D-3512 | S-1009 | 1391 | GUUUAUGAACU | 1023 | A-2000 | 1391 | UUAACGUCAGU | 914 |
| | | | GAUCUUAACC | | | | UCAUAAACUU | |
| D-3513 | S-1012 | 1391 | GUUUAUGAACU | 1027 | A-2000 | 1391 | UUAACGUCAGU | 914 |
| | | | GAUCUUGCCC | | | | UCAUAAACUU | |
| D-3514 | S-1013 | 1391 | GUUUAUGAACU | 1028 | A-2000 | 1391 | UUAACGUCAGU | 914 |
| | | | GAUCUUGGCC | | | | UCAUAAACUU | |
| D-3515 | S-1014 | 1391 | GUUUAUGAACU | 1029 | A-2000 | 1391 | UUAACGUCAGU | 914 |
| | | | GAUCUUGACC | | | | UCAUAAACUU | |
| D-3516 | S-1018 | 1429 | CCACAAUGUUG | 1033 | A-2006 | 1428 | UCCGGUCACAA | 926 |
| | | | UGACUGGCCC | | | | CAUUGUGGUU | |
| D-3517 | S-1025 | 1429 | CCACAAUGUUG | 1040 | A-2006 | 1428 | UCCGGUCACAA | 926 |
| | | | UGACUGGGCC | | | | CAUUGUGGUU | |
| D-3518 | S-1032 | 1429 | CCACAAUGUUG | 1049 | A-2006 | 1428 | UCCGGUCACAA | 926 |
| | | | UGACUGGACC | | | | CAUUGUGGUU | |
| D-3519 | S-1039 | 1429 | CCACAAUGUAG | 1058 | A-2006 | 1428 | UCCGGUCACAA | 926 |
| | | | UGACUGGACC | | | | CAUUGUGGUU | |
| D-3520 | S-1001 | 2066 | GUGUUAGACGG | 1015 | A-2001 | 2066 | UGUCGGUACCG | 916 |
| | | | UACUGAUCCC | | | | UCUAACACUU | |
| D-3521 | S-1004 | 2066 | GUGUUAGACGG | 1018 | A-2001 | 2066 | UGUCGGUACCG | 916 |
| | | | UACUGAUGCC | | | | UCUAACACUU | |
| D-3522 | S-1007 | 2066 | GUGUUAGACCG | 1021 | A-2001 | 2066 | UGUCGGUACCG | 916 |
| | | | UACUGAUACC | | | | UCUAACACUU | |
| D-3523 | S-1010 | 2066 | GUGUUAGACGG | 1024 | A-2001 | 2066 | UGUCGGUACCG | 916 |
| | | | UACUGAUACC | | | | UCUAACACUU | |
| D-3524 | S-1021 | 2079 | CCGACAACCAG | 1036 | A-2009 | 2078 | UCCAAAUACUG | 932 |
| | | | UAUUUGGCCC | | | | GUUGUCGGUU | |
| D-3525 | S-1028 | 2079 | CCGACAACCAG | 1043 | A-2009 | 2078 | UCCAAAUACUG | 932 |
| | | | UAUUUGGGCC | | | | GUUGUCGGUU | |
| D-3526 | S-1035 | 2079 | CCGACAACCAG | 1053 | A-2009 | 2078 | UCCAAAUACUG | 932 |
| | | | UAUUUGGACC | | | | GUUGUCGGUU | |
| D-3527 | S-1042 | 2079 | CCGACAACCUG | 1061 | A-2009 | 2078 | UCCAAAUACUG | 932 |
| | | | UAUUUGGACC | | | | GUUGUCGGUU | |
| D-3528 | S-1019 | 4544 | GUCACAAAGAA | 1034 | A-2007 | 4544 | UUGCACGGUUC | 928 |
| | | | CCGUGUACCC | | | | UUUGUGACUU | |
| D-3529 | S-1026 | 4544 | GUCACAAAGAA | 1041 | A-2007 | 4544 | UUGCACGGUUC | 928 |
| | | | CCGUGUAGCC | | | | UUUGUGACUU | |
| D-3530 | S-1033 | 4544 | GUCACAAAGAA | 1050 | A-2007 | 4544 | UUGCACGGUUC | 928 |
| | | | CCGUGUAACC | | | | UUUGUGACUU | |
| D-3531 | S-1040 | 4544 | GUCACAAAGUA | 1059 | A-2007 | 4544 | UUGCACGGUUC | 928 |
| | | | CCGUGUAACC | | | | UUUGUGACUU | |
| D-3532 | S-1020 | 4597 | UGAACCUCUUG | 1035 | A-2008 | 4597 | UUUUAUAACAA | 930 |
| | | | UUAUAAACCC | | | | GAGGUUCAUU | |
| D-3533 | S-1027 | 4597 | UGAACCUCUUG | 1042 | A-2008 | 4597 | UUUUAUAACAA | 930 |
| | | | UUAUAAAGCC | | | | GAGGUUCAUU | |
| D-3534 | S-1034 | 4597 | UGAACCUCUUG | 1051 | A-2008 | 4597 | UUUUAUAACAA | 930 |
| | | | UUAUAAAACC | | | | GAGGUUCAUU | |
| D-3535 | S-1041 | 4597 | UGAACCUCUAG | 1060 | A-2008 | 4597 | UUUUAUAACAA | 930 |
| | | | UUAUAAAACC | | | | GAGGUUCAUU | |
| D-3536 | S-1044 | 4861 | GAUCAUUGGAA | 1063 | A-2011 | 4860 | UUUAGGAAUUC | 936 |
| | | | UUCCUAAUCC | | | | CAAUGAUCUU | |
| D-3537 | S-1046 | 4861 | GAUCAUUGGAA | 1065 | A-2011 | 4860 | UUUAGGAAUUC | 936 |
| | | | UUCCUAAGCC | | | | CAAUGAUCUU | |
| D-3538 | S-1048 | 4861 | GAUCAUUGGAA | 1068 | A-2011 | 4860 | UUUAGGAAUUC | 936 |
| | | | UUCCUAAACC | | | | CAAUGAUCUU | |
| D-3539 | S-1050 | 4861 | GAUCAUUGGUA | 1071 | A-2011 | 4860 | UUUAGGAAUUC | 936 |
| | | | UUCCUAAACC | | | | CAAUGAUCUU | |
| D-3540 | S-1043 | 4864 | CAUUGGAAUUC | 1062 | A-2010 | 4864 | UAUUUUAGGAA | 934 |
| | | | CUAAAAUUCC | | | | UUCCAAUGUU | |
| D-3541 | S-1045 | 4864 | CAUUGGAAUUC | 1064 | A-2010 | 4864 | UAUUUUAGGAA | 934 |
| | | | CUAAAAUGCC | | | | UUCCAAUGUU | |
| D-3542 | S-1047 | 4864 | CAUUGGAAUUC | 1066 | A-2010 | 4864 | UAUUUUAGGAA | 934 |
| | | | CUAAAAUACC | | | | UUCCAAUGUU | |
| D-3543 | S-1049 | 4864 | CAUUGGAAUAC | 1070 | A-2010 | 4864 | UAUUUUAGGAA | 934 |
| | | | CUAAAAUACC | | | | UUCCAAUGUU | |
| D-3544 | S-1015 | 6188 | GCAAUUCAGUC | 1030 | A-2003 | 6188 | UACAACGAGAC | 920 |
| | | | UCGUUGUCCC | | | | UGAAUUGCUU | |
| D-3545 | S-1022 | 6188 | GCAAUUCAGUC | 1037 | A-2003 | 6188 | UACAACGAGAC | 920 |
| | | | UCGUUGUGCC | | | | UGAAUUGCUU | |
| D-3546 | S-1029 | 6188 | GCAAUUCAGUC | 1044 | A-2003 | 6188 | UACAACGAGAC | 920 |
| | | | UCGUUGUACC | | | | UGAAUUGCUU | |
| D-3547 | S-1036 | 6188 | GCAAUUCAGAC | 1055 | A-2003 | 6188 | UACAACGAGAC | 920 |
| | | | UCGUUGUACC | | | | UGAAUUGCUU | |
| D-3548 | S-1002 | 6751 | CCUGCUAGCUC | 1016 | A-2002 | 6751 | UAAGCAUGGAG | 918 |
| | | | CAUGCUUCCC | | | | CUAGCAGGUU | |
| D-3549 | S-1005 | 6751 | CCUGCUAGCUC | 1019 | A-2002 | 6751 | UAAGCAUGGAG | 918 |
| | | | CAUGCUUGCC | | | | CUAGCAGGUU | |
| D-3550 | S-1008 | 6751 | CCUGCUAGCAC | 1022 | A-2002 | 6751 | UAAGCAUGGAG | 918 |
| | | | CAUGCUUACC | | | | CUAGCAGGUU | |
| D-3551 | S-1011 | 6751 | CCUGCUAGCUC | 1025 | A-2002 | 6751 | UAAGCAUGGAG | 918 |
| | | | CAUGCUUACC | | | | CUAGCAGGUU | |

TABLE 5
Sense and antisense strand sequences of HTT dsRNA

| | | | Sense | | | | Antisense | |
|---|---|---|---|---|---|---|---|---|
| siRNA | | | Strand | SS | | | Strand | AS |
| Duplex | | Start | Sequence | SEQ | | Start | Sequence | SEQ |
| ID | SS ID | SS | (5′-3′) | ID | AS ID | AS | (5′-3′) | ID |
| D-3552 | S-1051dt | 1391 | GUUUAUGAACUG | 1072 | A- | 1391 | UUAACGUCAGU | 915 |
| | | | ACGUUAAdTdT | | 2000dt | | UCAUAAACdTdT | |
| D-3553 | S-1052dt | 2066 | GUGUUAGACGGU | 1073 | A- | 2066 | UGUCGGUACCG | 917 |
| | | | ACCGACAdTdT | | 2001dt | | UCUAACACdTdT | |
| D-3554 | S-1011dt | 6751 | CCUGCUAGCUCC | 1026 | A- | 6751 | UAAGCAUGGAG | 919 |
| | | | AUGCUUAdTdT | | 2002dt | | CUAGCAGGdTdT | |
| D-3555 | S-1053dt | 10322 | AUAUCAGUAAAG | 1074 | A- | 10322 | UUAAUCUCUUU | 938 |
| | | | AGAUUAAdTdT | | 2012dt | | ACUGAUAUdTdT | |
| D-3556 | S-1030dt | 1386 | UCCAGGUUUAUG | 1047 | A- | 1386 | UUCAGUUCAUA | 923 |
| | | | AACUGAAdTdT | | 2004dt | | AACCUGGAdTdT | |
| D-3557 | S-1054dt | 1390 | GGUUUAUGAACU | 1075 | A- | 1390 | UAACGUCAGUU | 925 |
| | | | GACGUUAdTdT | | 2005dt | | CAUAAACCdTdT | |
| D-3558 | S-1055dt | 1429 | CCACAAUGUUGU | 1076 | A- | 1429 | UCCGGUCACAA | 927 |
| | | | GACCGGAdTdT | | 2006dt | | CAUUGUGGdTdT | |
| D-3559 | S-1035dt | 2079 | CCGACAACCAGU | 1054 | A- | 2079 | UCCAAAUACUG | 933 |
| | | | AUUUGGAdTdT | | 2009dt | | GUUGUCGGdTdT | |
| D-3560 | S-1056dt | 4544 | GUCACAAAGAAC | 1077 | A- | 4544 | UUGCACGGUUC | 929 |
| | | | CGUGCAAdTdT | | 2007dt | | UUUGUGACdTdT | |
| D-3561 | S-1034dt | 4597 | UGAACCUCUUGU | 1052 | A- | 4597 | UUUUAUAACAA | 931 |
| | | | UAUAAAAdTdT | | 2008dt | | GAGGUUCAdTdT | |
| D-3562 | S-1029dt | 6188 | GCAAUUCAGUCU | 1045 | A- | 6188 | UACAACGAGAC | 921 |
| | | | CGUUGUAdTdT | | 2003dt | | UGAAUUGCdTdT | |
| D-3563 | S-1047dt | 4864 | CAUUGGAAUUCC | 1067 | A- | 4864 | UAUUUUAGGAA | 935 |
| | | | UAAAAUAdTdT | | 2010dt | | UUCCAAUGdTdT | |
| D-3564 | S-1048dt | 4861 | GAUCAUUGGAAU | 1069 | A- | 4861 | UUUAGGAAUUC | 937 |
| | | | UCCUAAAdTdT | | 2011dt | | CAAUGAUCdTdT | |
| D-3565 | S-1057dt | 4864 | CAUUGGAAUUCC | 1078 | A- | 4864 | GAUUUUAGGAA | 939 |
| | | | UAAAAUCdTdT | | 2013dt | | UUCCAAUGdTdT | |

TABLE 6
Antisense and Sense strand sequences of HTT dsRNA

| | | | Antisense | | | | Sense | |
|---|---|---|---|---|---|---|---|---|
| siRNA | | | Strand | AS | | | Strand | SS |
| Duplex | | Start | Sequence | SEQ | | Start | Sequence | SEQ |
| ID | AS ID | AS | (5′-3′) | ID | SS ID | SS | (5′-3′) | ID |
| D-3569 | S-1060 | 6751 | CCUGCUAGCUC | 1081 | A-2015 | 6751 | UAAGCAUGGAG | 941 |
| | | | CAUGCUUAUU | | | | CUAGCAGGGU | |
| D-3570 | S-1061 | 6751 | CCUGCUAGCUC | 1082 | A-2002 | 6751 | UAAGCAUGGAG | 918 |
| | | | CAUGCUUGUU | | | | CUAGCAGGUU | |

In other embodiments, the siRNA molecules of the present disclosure can be encoded in plasmid vectors, AAV particles, viral genome or other nucleic acid expression vectors for delivery to a cell.

DNA expression plasmids can be used to stably express the siRNA duplexes or dsRNA of the present disclosure in cells and achieve long-term inhibition of the target gene expression. in one aspect, the sense and antisense strands of a siRNA duplex are typically linked by a short spacer sequence leading to the expression of a stem-loop structure termed short hairpin RNA (shRNA). The hairpin is recognized and cleaved by Dicer, thus generating mature siRNA molecules.

According to the present disclosure, AAV particles comprising the nucleic acids encoding the siRNA molecules targeting HTT mRNA are produced, the AAV serotypes may be any of the serotypes listed in Table 1. Non-limiting examples of the AAV serotypes include, AAV1, AAV2, AAV3, AAV4, AAV5, AAV6, AAV7, AAV8, AAV9, AAV9.47, AAV9(hu14), AAV10, AAV11, AAV12, AAVrh8, AAVrh10, AAV-DJ8, AAV-DJ, AAV-PHP.A, and/or AAV-PHP.B, and variants thereof.

In some embodiments, the siRNA duplexes or encoded dsRNA of the present disclosure suppress (or degrade) target mRNA (e.g., HTT), Accordingly, the siRNA duplexes or encoded dsRNA can be used to substantially inhibit HTT gene expression in a cell, for example a neuron. In some aspects, the inhibition of HTT gene expression refers to an inhibition by at least about 20%, preferably by at least about 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95(N), 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%. Accordingly, the protein product of the targeted gene may be inhibited by at least about 20%, preferably by at least about 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%.

According to the present disclosure, the siRNA molecules are designed and tested for their ability in reducing HTT mRNA levels in cultured cells. Non-limiting examples of cultured cells include HEK293, HeLa, primary astrocytes, U251 astrocytes, SH-SY5Y neuron cells, FRhK-4 rhesus macaque (Macaca mulatta) kidney cells, and fibroblasts from HD patients. Such siRNA molecules may form a duplex such as, but not limited to, include those listed in Table 4, Table 5 or Table 6. As a non-limiting example, the siRNA duplexes may be siRNA duplex IDs: D-3500 to D-3570.

In some embodiments, the siRNA molecules comprise a miRNA seed match for the target (e.g.,) located in the guide strand. In another embodiment, the siRNA molecules comprise a miRNA seed match for the target (e.g., HTT) located in the passenger strand. In yet another embodiment, the siRNA duplexes or encoded &RNA targeting HTT gene do not comprise a seed match for the target e.g., HTT) located in the guide or passenger strand.

In some embodiments, the siRNA duplexes or encoded dsRNA targeting HTT gene may have almost no significant full-length off target effects for the guide strand. In another embodiment, the siRNA. duplexes or encoded dsRNA targeting HTT gene may have almost no significant full-length off target effects for the passenger strand. The siRNA duplexes or encoded dsRNA targeting HTT gene may have less than 1%, 2%, 3%, 4 0, 5%, 6%, 7%, 8%, 9%, 10%, 11%, 12%, 13%, 14%, 15%, 20%, 25%, 30%, 35%, 40%, 45%, 50%, 1-5%, 2-6%, 3-7%, 4-8%, 5-9%, 5-10%, 6-10%, 5-15%, 5-20%, 5-25%, 5-30%, 10-20%, 10-30%, 10-40%, 10-50%, 15-30%, 15-40%, 15-45%, 20-40%, 20-50%, 25-50%, 30-40%, 30-50%, 35-50%, 40-50%, 45-50% full- length off target effects for the passenger strand. In yet another embodiment, the siRNA duplexes or encoded dsRNA targeting HTT gene may have almost no significant full-length off target effects for the guide strand or the passenger strand. The siRNA duplexes or encoded dsRNA targeting HTT gene may have less than 1%, 2%, 3%, 4%, 5%, 6%, 7%, 8%, 9%, 10%, 11%, 12%, 13%, 14%, 15%, 20%, 25%, 30%, 35%, 40%, 45%, 50%, 1-5%, 2-6%, 3-7%, 4-8%, 5-9%, 5-10%, 6-10%, 5-15%, 5-20%, 5-25%, 5-30%, 10-20%, 10-30%, 10-40%, 10-50%, 15-30%, 15-40%, 15-45%, 20-40%, 20-50%, 25-50%, 30-40%, 30-50%, 35-50%, 40-50%, 45-50% full-length off target effects for the guide or passenger strand.

In some embodiments, the siRNA duplexes or encoded dsRNA targeting HTT gene may have high activity in vitro. In another embodiment, the siRNA molecules may have low activity in vitro. In yet another embodiment, the siRNA duplexes or dsRNA targeting the HTT gene may have high guide strand activity and low passenger strand activity in vitro.

In some embodiments, the siRNA molecules have a high guide strand activity and low passenger strand activity in vitro. The target knock-down (KD) by the guide strand may be at least 30%, 40%, 50%, 60%, 65%, 70%, 75%, 80%, 85%, 90%, 95%, 99%, 99.5% or 100%. The target knock-down by the guide strand may be 30-40%, 35-40%, 40-50%, 45-50%, 50-55%, 50-60%, 60-65%, 60-70%, 60-75%, 60-80%, 60-85%, 60-90%, 60-95%, 60-99%, 60-99.5%, 60-100%, 65-70%, 65-75%, 65-80%, 65-85%, 65-90%, 65-95%, 65-99%, 65-99.5%, 65-100%, 70-75%, 70-80%, 70-85%, 70-90%, 70-95%, 70-99%, 70-99.5%, 70-100%, 75-80%, 75-85%, 75-90%, 75-95%, 75-99%, 75-99.5 0 0, 75-100%, 80-85%, 80-90%, 80-95%, 80-99%, 80-99.5%, 80-100%, 85-90%, 85-95%, 85-99%, 85-99.5%, 85-100%, 90-95%, 90-99%, 90-99.5%, 90-100%, 95-99%, 95-99.5%, 95-100%, 99-99.5 99-100% or 99.5-100%. As a non-limiting example, the target knock-down (KD) by the guide strand is greater than 70%. As a non-limiting example, the target knock-down (KD) by the guide strand is greater than 60%.

In some embodiments, the siRNA duplex is designed so there is no miRNA seed match for the sense or antisense sequence to non-Htt sequence.

In some embodiments, the IC₅₀ of the guide strand for the nearest off target is greater than 100 multiplied by the IC₅₀ of the guide strand for the on-target gene, Htt. As a non-limiting example, if the IC₅₀ of the guide strand for the nearest off target is greater than 100 multiplied by the IC₅₀ of the guide strand for the target then the siRNA molecule is said to have high guide strand selectivity for inhibiting Htt in vitro.

In some embodiments, the 5′ processing of the guide strand has a correct start (n) at the 5′ end at least 75%, 80%, 85%, 90%, 95%, 99% or 100% of the time in vitro or in vivo. As a non-limiting example, the 5′ processing of the guide strand is precise and has a correct start (n) at the 5′ end at least 99% of the time in vitro. As a non-limiting example, the 5′ processing of the guide strand is precise and has a correct start (n) at the 5′ end at least 99° of the time in vivo. As a non-limiting example, the 5′ processing of the guide strand is precise and has a correct start (n) at the 5′ end at least 90% of the time in vitro. As a non-limiting example, the 5′ processing of the guide strand is precise and has a correct start (n) at the 5′ end at least 90% of the time in vivo. As a non-limiting example, the 5′ processing of the guide strand is precise and has a correct start (n) at the 5′ end at least 85% of the time in vitro. As a non-limiting example, the 5′ processing of the guide strand is precise and has a correct start (n) at the 5′ end at least 85% of the time in vivo.

In some embodiments, the guide to passenger (G:P) (also referred to as the antisense to sense) strand ratio expressed is 1:10, 1:9, 1:8, 1:7, 1:6, 1:5, 1:4, 1:3, 1:2, 1;1, 2:10, 2:9, 2:8, 2:7, 2:6, 2:5, 2:4, 2:3, 2:2, 2:1, 3:10, 3:9, 3:8, 3:7, 3:6, 3:5, 3:4, 3:3, 3:2, 3:1, 4:10, 4:9, 4:8, 4:7, 4:6, 4:5, 4:4, 4:3, 4:2, 4:1, 5:10, 5:9, 5:8, 5:7, 5:6, 5:5, 5:4, 5:3, 5:2, 5:1, 6:10, 6:9, 6:8, 6:7, 6:6, 6:5, 6:4, 6:3, 6:2, 6:1, 7:10, 7:9, 7:8, 7:7, 7:6, 7:5, 7:4, 7:3, 7:2, 7:1, 8:10, 8:9, 8:8, 8:7, 8:6, 8:5, 8:4, 8:3, 8:2, 8:1, 9:10, 9:9, 9:8, 9:7, 9:6, 9:5, 9:4, 9:3, 9:2, 9:1, 10:10, 10:9, 10:8, 10:7, 10:6, 10:5, 10:4, 10:3, 10:2, 10:1, 1:99, 5:95, 10:90, 15:85, 20:80, 25:75, 30:70, 35:65, 40:60, 45:55, 50:50, 55:45, 60:40, 65:35, 70:30, 75:25, 80:20, 85:15, 90:10, 95:5, or 99:1 in vitro or in vivo. The guide to passenger ratio refers to the ratio of the guide strands to the passenger strands after intracellular processing of the pri-microRNA. For example, a 80:20 guide-to-passenger ratio would have 8 guide strands to every 2 passenger strands processed from the precursor. As a non-limiting example, the guide-to-passenger strand ratio is 8:2 in vitro. As a non-limiting example, the guide-to-passenger strand ratio is 8:2 in vivo. As a non-limiting example, the guide-to-passenger strand ratio is 9:1 in vitro. As a non-limiting example, the guide-to-passenger strand ratio is 9:1 in vivo.

In some embodiments, the guide to passenger (G:P) (also referred to as the antisense to sense) strand ratio expressed is greater than 1.

In some embodiments, the guide to passenger (G:P) (also referred to as the antisense to sense) strand ratio expressed is greater than 2.

In some embodiments, the guide to passenger (G:P) (also referred to as the antisense to sense) strand ratio expressed is greater than 5.

In some embodiments, the guide to passenger (G:P) (also referred to as the antisense to sense) strand ratio expressed is greater than 10.

In some embodiments, the guide to passenger (G:P) (also referred to as the antisense to sense) strand ratio expressed is greater than 20.

In some embodiments, the guide to passenger (G:P) (also referred to as the antisense to sense) strand ratio expressed is greater than 50.

In some embodiments, the guide to passenger (G:P) (also referred to as the antisense to sense) strand ratio expressed is at least 3:1.

In some embodiments, the guide to passenger (G:P) (also referred to as the antisense to sense) strand ratio expressed is at least 5:1.

In some embodiments, the guide to passenger (G:P) (also referred to as the antisense to sense) strand ratio expressed is at least 10:1.

in some embodiments, the guide to passenger (G:P) (also referred to as the antisense to sense) strand ratio expressed is at least 20:1.

In some embodiments, the guide to passenger (G:P) (also referred to as the antisense to sense) strand ratio expressed is at least 50:1.

In some embodiments, the passenger to guide (P:G) (also referred to as the sense to antisense) strand ratio expressed is 1:10, 1:9, 1:8, 1:7, 1:6, 1:5, 1:4, 1:3, 1:2, 1;1, 2:10, 2:9, 2:8, 2:7, 2:6, 2:5, 2:4, 2:3, 2:2, 2:1, 3:10, 3:9, 3:8, 3:7, 3:6, 3:5, 3:4, 3:3, 3:2, 3:1, 4:10, 4:9, 4:8, 4:7, 4:6, 4:5, 4:4, 4:3, 4:2, 4:1, 5:10, 5:9, 5:8, 5:7, 5:6, 5:5, 5:4, 5:3, 5:2, 5:1, 6:10, 6:9, 6:8, 6:7, 6:6, 6:5, 6:4, 6:3, 6:2, 6:1, 7:10, 7:9, 7:8, 7:7, 7:6, 7:5, 7:4, 7:3, 7:2, 7:1, 8:10, 8:9, 8:8, 8:7, 8:6, 8:5, 8:4, 8:3, 8:2, 8:1, 9:10, 9:9, 9:8, 9:7, 9:6, 9:5, 9:4, 9:3, 9:2, 9:1, 10:10, 10:9, 10:8, 10:7, 10:6, 10:5, 10:4, 10:3, 10:2, 10:1, 1:99, 5:95, 10:90, 15:85, 20:80, 25:75, 30:70, 35:65, 40:60, 45:55, 50:50, 55:45, 60:40, 65:35, 70:30, 75:25, 80:20, 85:15, 90:10, 95:5, or 99:1 in vitro or in vivo. The passenger to guide ratio refers to the ratio of the passenger strands to the guide strands after the intracellular processing of the pri-microRNA. For example, a 80:20 of passenger-to-guide ratio would have 8 passenger strands to every 2 guide strands processed from the precursor. As a non-limiting example, the passenger-to-guide strand ratio is 80:20 in vitro. As a non-limiting example, the passenger-to-guide strand ratio is 80:20 in vivo. As a non-limiting example, the passenger-to-guide strand ratio is 8:2 in vitro. As a non-limiting example, the passenger-to-guide strand ratio is 8:2 in vivo. As a non-limiting example, the passenger-to-guide strand ratio is 9:1 in vitro. As a non-limiting example, the passenger-to-guide strand ratio is 9:1 in vivo.

In some embodiments, the passenger to guide (P:G) (also referred to as the sense to antisense) strand ratio expressed is greater than 1.

In some embodiments, the passenger to guide (P:G) (also referred to as the sense to antisense) strand ratio expressed is greater than 2.

In some embodiments, the passenger to guide (P:G) (also referred to as the sense to antisense) strand ratio expressed is greater than 5.

In some embodiments, the passenger to guide (P:G) (also referred to as the sense to antisense) strand ratio expressed is greater than 10.

In some embodiments, the passenger to guide (P:G) (also referred to as the sense to antisense) strand ratio expressed is greater than 20.

In some embodiments, the passenger to guide (P:G) (also referred to as the sense to antisense) strand ratio expressed is greater than 50.

In some embodiments, the passenger to guide (P:G) (also referred to as the sense to antisense) strand ratio expressed is at least 3:1.

In some embodiments, the passenger to guide (P:G) (also referred to as the sense to antisense) strand ratio expressed is at least 5:1,

In some embodiments, the passenger to guide (P:G) (also referred to as the sense to antisense) strand ratio expressed is at least 10:1.

In some embodiments, the passenger to guide (P:G) (also referred to as the sense to antisense) strand ratio expressed is at least 20:1.

In some embodiments, the passenger to guide (P:G) (also referred to as the sense to antisense) strand ratio expressed is at least 50:1.

In some embodiments, a passenger-guide strand duplex is considered effective when the pri- or pre-microRNAs demonstrate, but methods known in the art and described herein, greater than 2-fold guide to passenger strand ratio when processing is measured. As a non-limiting examples, the pri- or pre-microRNAs demonstrate great than 2-fold, 3-fold, 4-fold, 5-fold, 6-fold, 7-fold, 8-fold, 9-fold, 10-fold, 11-fold, 12-fold, 13-fold, 14-fold, 15-fold, or 2 to 5-fold, 2 to 10-fold, 2 to 15-fold, 3 to 5-fold, 3 to 10-fold, 3 to 15-fold, 4 to 5-fold, 4 to 10-fold, 4 to 15-fold, 5 to 10-fold, 5 to 15-fold, 6 to 10-fold, 6 to 15-fold, 7 to 10-fold, 7 to 15-fold, 8 to 10-fold, 8 to 15-fold, 9 to 10-fold, 9 to 15-fold, 10 to 15-fold, 11 to 15-fold, 12 to 15-fold, 13 to 15-fold, or 14 to 15-fold guide to passenger strand ratio when processing is measured.

In some embodiments, the vector genome encoding the dsRNA comprises a sequence which is at least 60%, 65%, 70%, 75%, 80%, 85%, 90%, 95%, 99% or more than 99% of the full length of the construct. As a non-limiting example, the vector genome comprises a sequence which is at least 80% of the full-length sequence of the construct.

In some embodiments, the siRNA molecules may be used to silence wild type and/or mutant HTT by targeting at least one exon on the htt sequence. The exon may be exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, exon 19, exon 20, exon 21, exon 22, exon 23, exon 24, exon 25, exon 26, exon 27, exon 28, exon 29, exon 30, exon 31, exon 32, exon 33, exon 34, exon 35, exon 36, exon 37, exon 38, exon 39, exon 40, exon 41, exon 42, exon 43, exon 44, exon 45, exon 46, exon 47, exon 48, exon 49, exon 50, exon 51, exon 52, exon 53, exon 54, exon 55, exon 56, exon 57, exon 58, exon 59, exon 60, exon 61, exon 62, exon 63, exon 64, exon 65, exon 66, and/or exon 67. As a non-limiting example, the siRNA molecules may be used to silence wild type and/or mutant HTT by targeting exon 1. As another non-limiting example, the siRNA molecules may be used to silence wild type and/or mutant HTT by targeting an exon other than exon 1. As another non-limiting example, the siRNA molecules may be used to silence wild type and/or mutant WIT by targeting exon 50. As another non-limiting example, the siRNA molecules may be used to silence wild type and/or mutant HTT by targeting exon 67.

### Molecular Scaffold

In some embodiments, the siRNA molecules may be encoded in a modulatory polynucleotide which also comprises a molecular scaffold. As used herein a “molecular scaffold” is a framework or starting molecule that forms the sequence or structural basis against which to design or make a subsequent molecule.

In some embodiments, the molecular scaffold comprises at least one 5′ flanking region. As a non-limiting example, the 5′ flanking region may comprise a 5′ flanking sequence which may be of any length and may be derived in whole or in part from wild type microRNA sequence or be a completely artificial sequence.

In some embodiments, the molecular scaffold comprises at least one 3′ flanking region. As a non-limiting example, the 3′ flanking region may comprise a 3′ flanking sequence which may be of any length and may be derived in whole or in part from wild type microRNA sequence or be a completely artificial sequence.

In some embodiments, one or both of the and 3′ flanking sequences are absent.

In some embodiments the 5′ and 3′ flanking sequences are the same length.

In some embodiments the 5′ flanking sequence is from 1-10 nucleotides in length, from 5-15 nucleotides in length, from 10-30 nucleotides in length, from 20-50 nucleotides in length, greater than 40 nucleotides in length, greater than 50 nucleotides in length, greater than 100 nucleotides in length or greater than 200 nucleotides in length.

In some embodiments, the 5′ flanking sequence may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 nucleotides in length.

In some embodiments the 3′ flanking sequence is from 1-10 nucleotides in length, from 5-15 nucleotides in length, from 10-30 nucleotides in length, from 20-50 nucleotides in length, greater than 40 nucleotides in length, greater than 50 nucleotides in length, greater than 100 nucleotides in length or greater than 200 nucleotides in length.

In some embodiments, the 3′ flanking sequence may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 nucleotides in length.

In some embodiments, the molecular scaffold comprises at least one loop motif region. As a non-limiting example, the loop motif region may comprise a sequence which may be of any length.

In some embodiments, the molecular scaffold comprises a 5′ flanking region, a loop motif region and/or a 3′ flanking region.

In some embodiments, at least one siRNA, miRNA or other RNAi agent described herein, may be encoded by a modulatory polynucleotide which may also comprise at least one molecular scaffold. The molecular scaffold may comprise a 5′ flanking sequence which may be of any length and may be derived in whole or in part from wild type microRNA sequence or be completely artificial. The 3′ flanking sequence may mirror the 5′ flanking sequence and/or a 3′ flanking sequence in size and origin. Either flanking sequence may be absent. The 3′ flanking sequence may optionally contain one or more CNNC motifs, where “N” represents any nucleotide.

Forming the stem of a stem loop structure is a minimum of the modulatory polynucleotide encoding at least one siRNA, miRNA or other RNAi agent described herein. In some embodiments, the siRNA, miRNA or other RNAi agent described herein comprises at least one nucleic acid sequence which is in part complementary or will hybridize to a target sequence. In some embodiments the payload is an siRNA molecule or fragment of an siRNA molecule,

In some embodiments, the 5′ arm of the stem loop structure of the modulatory polynucleotide comprises a nucleic acid sequence encoding a sense sequence. Non-limiting examples of sense sequences, or fragments or variants thereof, which may be encoded by the modulatory polynucleotide are described in Table 3.

In some embodiments, the 3′ arm of the stem loop of the modulatory polynucleotide comprises a nucleic acid sequence encoding an antisense sequence. The antisense sequence, in some instances, comprises a “G” nucleotide at the 5′ most end. Non-limiting examples of antisense sequences, or fragments or variants thereof, which may be encoded by the modulatory polynucleotide are described in Table 2.

In other embodiments, the sense sequence may reside on the 3′ arm while the antisense sequence resides on the 5′ arm of the stem of the stem loop structure of the modulatory polynucleotide. Non-limiting examples of sense and antisense sequences which may be encoded by the modulatory polynucleotide are described in Tables 2 and 3.

In some embodiments, the sense and antisense sequences may be completely complementary across a substantial portion of their length. In other embodiments the sense sequence and antisense sequence may be at least 70, 80, 90, 95 or 99% complementarity across independently at least 50, 60, 70, 80, 85, 90, 95, or 99% of the length of the strands.

Neither the identity of the sense sequence nor the homology of the antisense sequence need to be 100% complementarity to the target sequence.

In some embodiments, separating the sense and antisense sequence of the stem loop structure of the modulatory polynucleotide is a loop sequence (also known as a loop motif, linker or linker motif). The loop sequence may be of any length, between 4-30 nucleotides, between 4-20 nucleotides, between 4-15 nucleotides, between 5-15 nucleotides, between 6-12 nucleotides, 6 nucleotides, 7 nucleotides, 8 nucleotides, 9 nucleotides, 10 nucleotides, 11 nucleotides, 12 nucleotides. 13 nucleotides, 14 nucleotides, and/or 15 nucleotides.

In some embodiments, the loop sequence comprises a nucleic acid sequence encoding at least one UGUG motif. In some embodiments, the nucleic acid sequence encoding the UGUG motif is located at the 5′ terminus of the loop sequence.

In some embodiments, spacer regions may be present in the modulatory polynucleotide to separate one or more modules (e.g., 5′ flanking region, loop motif region, 3′ flanking region, sense sequence, antisense sequence) from one another. There may be one or more such spacer regions present.

In some embodiments, a spacer region of between 8-20, i.e., 8, 9, 10, 11, 12, 13, 14, 15, 16, 17, 18, 19, or 20 nucleotides may be present between the sense sequence and a flanking region sequence.

In some embodiments, the length of the spacer region is 13 nucleotides and is located between the 5′ terminus of the sense sequence and the 3′ terminus of the flanking sequence. In some embodiments, a spacer is of sufficient length to form approximately one helical turn of the sequence.

In some embodiments, a spacer region of between 8-20, i.e., 8, 9, 10, 11, 12, 13, 14, 15, 16, 17, 18, 19, or 20 nucleotides may be present between the antisense sequence and a flanking sequence.

In some embodiments, the spacer sequence is between 10-13, i.e., 10, 11, 12 or 13 nucleotides and is located between the 3′ terminus of the antisense sequence and the 5′ terminus of a flanking sequence. In some embodiments, a spacer is of sufficient length to form approximately one helical turn of the sequence.

In some embodiments, the molecular scaffold of the modulatory polynucleotide comprises in the 5′ to 3′ direction, a 5′ flanking sequence, a 5′ arm, a loop motif, a 3′ arm and a 3′ flanking sequence, As a non-limiting example, the 5′ arm may comprise a nucleic acid sequence encoding a sense sequence and the 3′ arm comprises a nucleic acid sequence encoding the antisense sequence. In another non-limiting example, the 5′ arm comprises a nucleic acid sequence encoding the antisense sequence and the 3′ arm comprises a nucleic acid sequence encoding the sense sequence.

In some embodiments, the 5′ arm, sense and/or antisense sequence, loop motif and/or 3′ arm sequence may be altered (e.g., substituting 1 or more nucleotides, adding nucleotides and/or deleting nucleotides). The alteration may cause a beneficial change in the function of the construct (e.g., increase knock-down of the target sequence, reduce degradation of the construct, reduce off target effect, increase efficiency of the payload, and reduce degradation of the payload).

In some embodiments, the molecular scaffold of the modulatory polynucleotides is aligned in order to have the rate of excision of the guide strand (also referred to herein as the antisense strand) be greater than the rate of excision of the passenger strand (also referred to herein as the sense strand). The rate of excision of the guide or passenger strand may he, independently, 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 99% or more than 99%. As a non-limiting example, the rate of excision of the guide strand is at least 80%. As another non-limiting example, the rate of excision of the guide strand is at least 90%.

In some embodiments, the rate of excision of the guide strand is greater than the rate of excision of the passenger strand. In one aspect, the rate of excision of the guide strand may be at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 99% or more than 99% greater than the passenger strand.

In some embodiments, the efficiency of excision of the guide strand is at least 60%, 65%, 70%, 75%, 80%, 85%, 90%, 95%, 99% or more than 99%. As a non-limiting example, the efficiency of the excision of the guide strand is greater than 80%.

In some embodiments, the efficiency of the excision of the guide strand is greater than the excision of the passenger strand from the molecular scaffold. The excision of the guide strand may be 2, 3, 4, 5, 6, 7, 8, 9, 10 or more than 10 times more efficient than the excision of the passenger strand from the molecular scaffold.

In some embodiments, the molecular scaffold comprises a dual function targeting modulatory polynucleotide. As used herein, a “dual-function targeting” modulatory polynucleotide is a polynucleotide where both the guide and passenger strands knock down the same target or the guide and passenger strands knock down different targets.

In some embodiments, the molecular scaffold of the modulatory polynucleotides described herein may comprise a 5′ flanking region, a loop motif region and a 3′ flanking region. Non-limiting examples of the sequences for the 5′ flanking region, loop motif region (may also be referred to as a linker region) and the 3′ flanking region which may be used, or fragments thereof used, in the modulatory polynucleotides described herein are shown in Tables 7-9.

TABLE 7
5′ Flanking Regions for Molecular Scaffold

| 5′ | | 5′ |
|---|---|---|
| Flanking | | Flanking |
| Region | 5′ Flanking | Region |
| Name | Region Sequence | SEQ ID |
| 5F3 | GTGCTGGGCGGGGGGCGGCGGGCCCTCCCGC | 1163 |
| | AGAACACCATGCGCTCCACGGAA | |
| 5F1 | GTGCTGGGCGGGGGGCGGCGGGCCCTCCCGC | 1161 |
| | AGAACACCATGCGCTCTTCGGAA | |
| 5F2 | GAAGCAAAGAAGGGGCAGAGGGAGCCCGTGA | 1162 |
| | GCTGAGTGGGCCAGGGACTGGGAGAAGGAGT | |
| | GAGGAGGCAGGGCCGGCATGCCTCTGCTGCT | |
| | GGCCAGA | |
| 5F4 | GGGCCCTCCCGCAGAACACCATGCGCTCCAC | 1164 |
| | GGAA | |
| 5F5 | CTCCCGCAGAACACCATGCGCTCCACGGAA | 1165 |
| 5F6 | GTGCTGGGCGGGGGGCGGCGGGCCCTCCCGC | 1166 |
| | AGAACACCATGCGCTCCACGGAAG | |
| 5F7 | GTGCTGGGCGGGGGGCGGCGGGCCCTCCCGC | 1167 |
| | AGAACACCATGCGCTCCTCGGAA | |

TABLE 8
Loop Motif Regions for Molecular Scaffold

| Loop | | Loop |
|---|---|---|
| Motif | | Motif |
| Region | Loop Motif | Region |
| Name | Region Sequence | SEQ ID |
| L5 | GTGGCCACTGAGAAG | 1172 |
| L1 | TGTGACCTGG | 1168 |
| L2 | TGTGATTTGG | 1169 |
| L3 | GTCTGCACCTGTCACTAG | 1170 |
| L4 | GTGACCCAAG | 1171 |
| L6 | GTGACCCAAT | 1173 |
| L7 | GTGACCCAAC | 1174 |
| L8 | GTGGCCACTGAGAAA | 1175 |

TABLE 9
3′ Flanking Regions for Molecular Scaffold

| 3′ | | 3′ |
|---|---|---|
| Flanking | | Flanking |
| Region | | Region |
| Name | 3′ Flanking Region Sequence | SEQ ID |
| 3F1 | CTGAGGAGCGCCTTGACAGCAGCCATG | 1178 |
| | GGAGGGCCGCCCCCTACCTCAGTGA | |
| 3F2 | CTGTGGAGCGCCTTGACAGCAGCCATG | 1179 |
| | GGAGGGCCGCCCCCTACCTCAGTGA | |
| 3F3 | TGGCCGTGTAGTGCTACCCAGCGCTGG | 1180 |
| | CTGCCTCCTCAGCATTGCAATTCCTCT | |
| | TCCCATCTGGGCACCAGTCAGCTACCC | |
| | TGGTGGGAATCTGGGTAGCC | |
| 3F4 | CTGAGGAGCGCCTTGACAGCAGCCATG | 1181 |
| | GGAGGGCC | |
| 3F5 | CTGCGGAGCGCCTTGACAGCAGCCATG | 1182 |
| | GGAGGGCCGCCCCCTACCTCAGTGA | |

In some embodiments, the molecular scaffold may comprise at least one 5′ flanking region, fragment or variant thereof listed in Table 7, As a non-limiting example, the 5′ flanking region may be 5F1, 5F2, 5F3, 5F4, 5F5, 5F6, or 5F7.

In some embodiments, the molecular scaffold may comprise at least one 5F1 flanking region.

In some embodiments, the molecular scaffold may comprise at least one 5F2 flanking region.

In some embodiments, the molecular scaffold may comprise at least one 5F3 flanking region.

In some embodiments, the molecular scaffold may comprise at least one 5F4 flanking region.

In some embodiments, the molecular scaffold may comprise at least one 5F5 flanking region.

In some embodiments, the molecular scaffold may comprise at least one 5F6 flanking region.

In some embodiments, the molecular scaffold may comprise at least one 5F7 flanking region,

In some embodiments, the molecular scaffold may comprise at least one loop motif region, fragment or variant thereof listed in Table 8. As a non-limiting example, the loop motif region may be L1, L2, L3, L4, L5, L6, L7, or L8.

In some embodiments, the molecular scaffold may comprise at least one L1 loop motif region.

In some embodiments, the molecular scaffold may comprise at least one L2 loop motif region.

In some embodiments, the molecular scaffold may comprise at least one L3 loop motif region.

In some embodiments, the molecular scaffold may comprise at least one L4 loop motif region.

In some embodiments, the molecular scaffold may comprise at least one L5 loop motif region.

In some embodiments, the molecular scaffold may comprise at least one L6 loop motif region.

In some embodiments, the molecular scaffold may comprise at least one L7 loop motif region.

In some embodiments, the molecular scaffold may comprise at least one L8 loop motif region.

In some embodiments, the molecular scaffold may comprise at least one 3′ flanking region, fragment or variant thereof listed in Table 9. As a non-limiting example, the 3′ flanking region may be 3F1, 3F2, 3F3, 3F4, or 3F5.

In some embodiments, the molecular scaffold may comprise at least one 3F1 flanking region.

In some embodiments, the molecular scaffold may comprise at least one 3F2 flanking region.

In some embodiments, the molecular scaffold may comprise at least one 3F3 flanking region.

in some embodiments, the molecular scaffold may comprise at least one 3F4 flanking region.

In some embodiments, the molecular scaffold may comprise at least one 3F5 flanking region.

In some embodiments, the molecular scaffold may comprise at least one 5′ flanking region, fragment or variant thereof, and at least one loop motif region, fragment or variant thereof, as described in Tables 7 and 8. As a non-limiting example, the 5′ flanking region and the loop motif region may be 5F1 and L1, 5F1 and L2, 5F1 and L3, 5F1 and L4, 5F1 and L5, 5F1 and L6, 5F1 and L7, 5F1 and L8, 5F2 and L1, 5F2 and L2, 5F2 and L3, 5F2 and L4, 5F2 and L5, 5F2 and L6, 5F2 and L7, 5F2 and L8, 5F3 and L1, 5F3 and L2, 5F3 and L3, 5F3 and L4, 5F3 and L5, 5F3 and L6, 5F3 and L7, 5F3 and L8, 5F4 and L1, 5F4 and L2, 5F4 and L3, 5F4 and L4, 5F4 and L5, 5F4 and L6, 5F4 and L7, 5F4 and L8, 5F5 and L1, 5F5 and L2, 5F5 and L3, 5F5 and L4, 5F5 and L5, 5F5 and L6, 5F5 and L7, 5F5 and L8, 5F6 and L1, 5F6 and L2, 5F6 and L3, 5F6 and L4, 5F6 and L5, 5F6 and L6, 5F6 and L7, 5F6 and L8, 5F7 and L1, 5F7 and L2, 5F7 and L3, 5F7 and L4, 5F7 and L5, 5F7 and L6, 5F7 and L7, and 5F7 and L8.

In some embodiments, the molecular scaffold may comprise at least one 5F2 flanking region and at least one L1 loop motif region.

In some embodiments, the molecular scaffold may comprise at least one 5F1 flanking region and at least one L4 loop motif region.

In some embodiments, the molecular scaffold may comprise at least one 5F7 flanking region and at least one L8 loop motif region,

In some embodiments, the molecular scaffold may comprise at least one 5F3 flanking region and at least one L4 loop motif region.

In some embodiments, the molecular scaffold may comprise at least one 5F3 flanking region and at least one L5 loop motif region.

In some embodiments, the molecular scaffold may comprise at least one 5F4 flanking region and at least one L4 loop motif region.

In some embodiments, the molecular scaffold may comprise at least one 5F3 flanking region and at least one L7 loop motif region,

In some embodiments, the molecular scaffold may comprise at least one 5F5 flanking region and at least one L4 loop motif region.

In some embodiments, the molecular scaffold may comprise at least one 5F6 flanking region and at least one L4 loop motif region.

In some embodiments, the molecular scaffold may comprise at least one 5F3 flanking region and at least one L6 loop motif region.

In some embodiments, the molecular scaffold may comprise at least one 5F7 flanking region and at least one L4 loop motif region.

In some embodiments, the molecular scaffold may comprise at least one 5F2 flanking region and at least one L2 loop motif region.

In some embodiments, the molecular scaffold may comprise at least one 5F1 flanking region and at least one L1 loop motif region.

In some embodiments, the molecular scaffold may comprise at least one 5F1 flanking region and at least one L2 loop motif region,

In some embodiments, the molecular scaffold may comprise at least one 3′ flanking region, fragment or variant thereof, and at least one motif region, fragment or variant thereof, as described in Tables 8 and 9. As a non-limiting example, the 3′ flanking region and the loop motif region may be 3F1 and L1, 3F1 and L2, 3F1 and L3, 3F1 and L4, 3F1 and L5, 3F1 and L6, 3F1 and L7, 3F1 and L8, 3F2 and L1, 3F2 and L2, 3F2 and L3, 3F2 and L4, 3F2 and L5, 3F2 and L6, 3F2 and L7, 3F2 and L8, 3F3 and L1, 3F3 and L2, 3F3 and L3, 3F3 and L4, 3F3 and L5, 3F3 and L6, 3F3 and L7, 3F3 and L8, 3F4 and L1, 3F4 and L2, 3F4 and L3, 3F4 and L4, 3F4 and L5, 3F4 and L6, 3F4 and L7, 3F4 and L8, 3F5 and L1, 3F5 and L2, 3F5 and L3, 3F5 and L4, 3F5 and L5, 3F5 and L6, 3F5 and L7, and 3F5 and L8.

In some embodiments, the molecular scaffold may comprise at least one L1 loop motif region and at least one 3F2 flanking region.

In some embodiments, the molecular scaffold may comprise at least one L4 loop motif region and at least one 3F1 flanking region.

In some embodiments, the molecular scaffold may comprise at least one L8 loop motif region and at least one 3F5 flanking region.

In some embodiments, the molecular scaffold may comprise at least one L5 loop motif region and at least 3F1 flanking region.

In some embodiments, the molecular scaffold may comprise at east one L4 loop motif region and at least one 3F4 flanking, region.

In some embodiments, the molecular scaffold may comprise at least one L7 loop motif region and at least one 3F1 flanking region.

In some embodiments, the molecular scaffold may comprise at least one L6 loop motif region and at least one 3F1 flanking region.

In some embodiments, the molecular scaffold may comprise at least one L4 loop motif region and at least one 3E5 flanking region.

In some embodiments, the molecular scaffold may comprise at least one L2 loop motif region and at least one 3F2 flanking region.

In some embodiments, the molecular scaffold may comprise at least one L1 loop motif region and at least one 3F3 flanking region.

In some embodiments, the molecular scaffold may comprise at least one L5 loop motif region and at least one 3F4 flanking region.

In some embodiments, the molecular scaffold may comprise at least one L1 loop motif region and at least one 3F1 flanking region.

In some embodiments, the molecular scaffold may comprise at least one L2 loop motif region and at least one 3F1 flanking region.

In some embodiments, the molecular scaffold may comprise at least one 5′ flanking region, fragment or variant thereof, and at least one 3′ flanking region, fragment or variant thereof, as described in Tables 7 and 9. As a non-limiting example, the flanking regions may he 5F1 and 3F1, 5F1 and 3F2, 5F1 and 3F3, 5F1 and 3F4, 5F1 and 5F5, 5F2 and 3F1, 5F2 and 3F2, 5F2 and 3F3, 5F2 and 3F4, 5F2 and 3F5, 5F3 and 3F1, 5F3 and 3F2, 5F3 and 3F3, 5F3 and 3F4, 5F3 and 3F5, 5F4 and 3F1, 5F4 and 3F2, 5F4 and 3F3, 5F4 and 3F4, 5F4 and 3F5, 5F5 and 3F1, 5F5 and 3F2, 5F5 and 3F3, 5F5 and 3F4, 5F5 and 3F5, 5F6 and 3F1, 5F6 and 3F2, 5F6 and 3F3, 5F6 and 3F4, 5F6 and 3F5, 5F7 and 3F1, 5F7 and 3F2, 5F7 and 3F3, 5F7 and 3F4, and 5F7 and 3F5.

In some embodiments, the molecular scaffold may comprise at least one 5F2 5′ flanking region and at least one 3F2 3′ flanking region.

In some embodiments, the molecular scaffold may comprise at least one 5F1 5′ flanking region and at least one 3F1 3′ flanking region.

In some embodiments, the molecular scaffold may comprise at least one 5F7 5′ flanking region and at least one 3F5 3′ flanking region.

In some embodiments, the molecular scaffold may comprise at east one 5F3 5′ flanking region and at least one 3F1 3′ flanking region.

In some embodiments, the molecular scaffold may comprise at least one 5F4 5′ flanking region and at least one 3F4 3′ flanking region.

In some embodiments, the molecular scaffold may comprise at least one 5F5 5′ flanking region and at least one 3F4 3′ flanking region,

In some embodiments, the molecular scaffold may comprise at least one 5F6 5′ flanking region and at least one 3F1 3′ flanking region.

In some embodiments, the molecular scaffold may comprise at least one 5F2 5′ flanking region and at least one 3F3 3′ flanking region.

In some embodiments, the molecular scaffold may comprise at least one 5F3 5′ flanking region and at least one 3F4 3′ flanking region.

In some embodiments, the molecular scaffold may comprise at least one 5F1 5′ flanking region and at least one 3F2 3′ flanking region.

In some embodiments, the molecular scaffold may comprise at least one 5′ flanking region, fragment or variant thereof, at least one loop motif region, fragment or variant thereof, and at least one 3′ flanking region as described in Tables 7-9. As a non-limiting example, the flanking and loop motif regions may be 5F1, L1 and 3F1; 5F1, L1 and 3F2; 5F1, L1 and 3F3; 5F1, L1 and 3F4; 5F1, L1 and 3F5; 5F2, L1 and 3F1; 5F2, L1 and 3F2; 5F2, L1 and 3F3; 5F2, L1 and 3F4; 5F2, L1 and 3F5; 5F3, L1 and 3F3; 5F3, L1 and 3F2; 5F3, L1 and 3F3; 5F3, L1 and 3F4; 5F3, L1 and 3F5; 5F4, L1 and 3F4; 5F4, L1 and 3F2; 5F4, L1 and 3F3; 5F4, L1 and 3F4; 5F4, L1 and 3F5; 5F5, L1 and 3F1; 5F5, L1 and 3F2; 5F5, L1 and 3F3; 5F5, L1 and 3F4; 5F5, L1 and 3F5; 5F6, L1 and 3F1; 5F6, L1 and 3F2; 5F6, L1 and 5F5; 5F6, L1 and 3F4; 5F6, L1 and 3F5; 5F7, L1 and 3F1; 5F7, L1 and 3F2; 5F7, L1 and 3F3; 5F7, L1 and 3F4; 5F7, L1 and 3F5; 5F1, L2 and 3F1; 5F1, L2 and 3F2; 5F1, L2 and 3F3; 5F1, L2 and 3F4; 5F1, L2 and 3F5; 5F2, L2 and 3F1; 5F2, L2 and 3F2; 5F2, L2 and 3F3; 5F2, L2 and 3F4; 5F2, L2 and 3F5; 5F3, L2 and 3F1; 5F3, L2 and 3F2; 5F3, L2 and 3F3; 5F3, L2 and 3F4; 5F3, L2 and 3F5; 5F4, L2 and 3F1; 5F4, L2 and 3F2; 5F4, L2 and 3F3; 5F4, L2 and 3F4; 5F4, L2 and 3F5; 5F5, L2 and 3F1; 5F5, L2 and 3F2; 5F5, L2 and 3F3; 5F5, L2 and 3F4; 5F5, L2 and 5F5; 5F6, L2 and 3F1; 5F6, L2 and 3F2; 5F6, L2 and 3F3; 5F6, L2 and 3F4; 5F6, L2 and 3F5; 5F7, L2 and 3F1; 5F7, L2 and 3F2; 5F7, L2 and 3F3; 5F7, L2 and 3F4; 5F7, L2 and 3F5; 5F1, L3 and 3F1; 5F1, L3 and 3F2; 5F1, L3 and 3F3; 5F1, L3 and 3F4; 5F1, L3 and 3F5; 5F2, L3 and 3F1; 5F2, L3 and 3F2; 5F2, L3 and 3F3; 5F2, L3 and 3F4; 5F2, L5 and 3F5; 5F3, L5 and 3F1; 5F3, L3 and 3F2; 5F3, L3 and 3F3; 5F3, L3 and 3F4; 5F3, L3 and 3F5; 5F4, L3 and 3F1; 5F4, L3 and 3F2; 5F4, L3 and 3F3; 5F4, L3 and 3F4; 5F4, L3 and 3F5; 5F5, L3 and 3F1; 5F5, L3 and 3F2; 5F5, L3 and 3F3; 5F5, L3 and 3F4; 5F5, L3 and 3F5; 5F6, L3 and 3F1; 5F6, L3 and 3F2; 5F6, L3 and 3F3; 5F6, L3 and 3F4; 5F6, L3 and 3F5; 5F7, L3 and 3F1; 5F7, L3 and 3F2; 5F7, L3 and 3F3; 5F7, L3 and 3F4; 5F7, L3 and 3F5; 5F1, L4 and 3F1; 5F1, L4 and 3F2; 5F1, L4 and 3F3; 5F1, L4 and 3F4; 5F1, L4 and 3F5; 5F2, L4 and 3F1; 5F2, L4 and 3F2; 5F2, L4 and 3F3; 5F2, L4 and 3F4; 5F2, L4 and 3F5; 5F3, L4 and 3F1; 5F, L4 and 3F2; 5F3, L4 and 3F3; 5F3, L4 and 3F4; 5F3, L4 and 3F5; 5F4, L4 and 3F1; 5F4, L4 and 3F2; 5F4, L4 and 3F3; 5F4, L4 and 3F4; 5F4, L4 and 3F5; 5F5, L4 and 3F1; 5F5, L4 and 3F2; 5F5, L4 and 3F3; 5F5, L4 and 3F4; 5F5, L4 and 3F5; 5F6, L4 and 3F1; 5F6, L4 and 3F2; 5F6, L4 and 3F3; 5F6, L4 and 3F4; 5F6, L4 and 3F5; 5F7, L4 and 3F1; 5F7, L4 and 3F2; 5F7, L4 and 3F3; 5F7, L4 and 3F4; 5F7, L4 and 3F5; 5F1, L5 and 5F1; 5F1, L5 and 3F2; 5F1, L5 and 3F3; 5F1, L5 and 3F4; 5F1, L5 and 3F5; 5F2, L5 and 3F1; 5F2, L5 and 3F2; 5F2, L5 and 3F3; 5F2, L5 and 3F4; 5F2, L5 and 3F5; 5F3, L5 and 3F1; 5F3, L5 and 3F2; 5F3, L5 and 3F3; 5F3, L5 and 3F4; 5F3, L5 and 3F5; 5F4, L5 and 3F1; 5F4, L5 and 3F2; 5F4, L5 and 3F3; 5F4, L5 and 3F4; 5F4, L5 and 3F5; 5F5, L5 and 3F1; 5F5, L5 and 3F2; 5F5, L5 and 3F3; 5F5, L5 and 3F4; 5F5, L5 and 3F5; 5F6, L5 and 3F1; 5F6, L5 and 3F2; 5F6, L5 and 3F3; 5F6, L5 and 3F4; 5F6, L5 and 3F5; 5F7, L5 and 3F1; 5F7, L5 and 3F2; 5F7, L5 and 3F3; 5F7, L5 and 3F4; 5F7, L5 and 3F5; 5F1, L6 and 5F1; 5F1, L6 and 3F2; 5F1, L6 and 3F3; 5F1, L6 and 3F4; 5F1, L6 and 3F5; 5F2, L6 and 3F1; 5F2, L6 and 3F2; 5F2, L6 and 3F3; 5F2, L6 and 3F4; 5F2, L6 and 3F5; 5F3, L6 and 3F1; 5F3, L6 and 3F2; 5F3, L6 and 3F3; 5F3, L6 and 3F4; 5F3, L6 and 3F5; 5F4, L6 and 3F1; 5F4, L6 and 3F2; 5F4, L6 and 3F3; 5F4, L6 and 3F4; 5F4, L6 and 3F5; 5F5, L6 and 3F1; 5F5, L6 and 3F2; 5F5, L6 and 3F3; 5F5, L6 and 3F4; 5F5, L6 and 3F5; 5F6, L6 and 3F1; 5F6, L6 and 3F2; 5F6, L6 and 3F3; 5F6, L6 and 3F4; 5F6, L6 and 3F5; 5F7, L6 and 3F1; 5F7, L6 and 3F2; 5F7, L6 and 3F3; 5F7, L6 and 3F4; 5F7, L6 and 3F5; 5F1, L7 and 3F1; 5F1, L7 and 3F2; 5F1, L7 and 3F3; 5F1, L7 and 3F4; 5F1, L7 and 3F5; 5F2, L7 and 3F1; 5F2, L7 and 3F2; 5F2, L7 and 3F3; 5F2, L7 and 3F4; 5F2, L7 and 3F5; 5F3, L7 and 3F1; 5F3, L7 and 3F2; 5F3, L7 and 3F3; 5F, L7 and 3F4; 5F3, L7 and 3F5; 5F4, L7 and 3F1; 5F4, L7 and 3F2; 5F4, L7 and 3F3; 5F4, L7 and 3F4; 5F4, L7 and 3F5; 5F5, L7 and 3F1; 5175, L7 and 3F2; 5F5, L7 and 3F3; 5F5, L7 and 3F4; 5F5, L7 and 3F5; 5F6, L7 and 3F1; 5F6, L7 and 3F2; 5F6, L7 and 3F3; 5F6, L7 and 3F4; 5F6, L7 and 3F5; 5F7, L7 and 3F1; 5F7, L7 and 3F2; 5F7, L7 and 3F3: 5F7, L7 and 3F4; 5F7, L7 and 3F5; 5F I, L8 and 3F1; 5F1, L8 and 3F2; 5F1, L8 and 3F3; 5F1, L8 and 3F4; 5F1, L8 and 3F5; 5F2, L8 and 3F1; 5F2, L8 and 3F2; 5F2, L8 and 3F3; 5F2, L8 and 3F4; 5F2, L8 and 3F5; 5F3, L8 and 3F1; 5F3, L8 and 3F2; 5F3, L8 and 3F3; 5F3, L8 and 3F4; 5F, L8 and 3F5; 5F4, L8 and 3F1; 5F4, L8 and 3F2; 5F4, L8 and 3F3; 5F4, L8 and 3F4; 5F4, L8 and 3F5; 5F5, L8 and 3F1; 5F5, L8 and 3F2; 5F5, L8 and 3F3; 5F5, L8 and 3F4; 5F5, L8 and 3F5; 5F6, L8 and 3F1; 5F6, L8 and 3F2; 5F6, L8 and 3F3; 5F6, L8 and 3F4; 5F6, L8 and 3F5; 5F7, L8 and 3F1; 5F7, L8 and 3F2; 5F7, L8 and 3F3; 5F7, L8 and 3F4; and 5F7, L8 and 3F5.

In some embodiments, the molecular scaffold may comprise at least one 5F2 5′ flanking region, at least one L1 loop motif region, and at least one 3F2 3′ flanking region.

In some embodiments, the molecular scaffold may comprise at least one 5F1 5′ flanking region, at least one L4 loop motif region, and at least one 3F1 3′ flanking region,

In some embodiments, the molecular scaffold may comprise at least one 5F7 5′ flanking region, at least one L8 loop motif region, and at least one 3F5 3′ flanking region.

In some embodiments, the molecular scaffold may comprise at least one 5F3 5′ flanking region, at least one L4 loop motif region, and at least one 3F1 3′ flanking region.

In some embodiments, the molecular scaffold may comprise at least one 5F3 5′ flanking region, at least one L5 loop motif region, and at least one 3F1 3′ flanking region.

In some embodiments, the molecular scaffold may comprise at least one 5F4 5′ flanking region, at least one L4 loop motif region, and at least one 3F4 3′ flanking region.

In some embodiments, the molecular scaffold may comprise at least one 5F3 5′ flanking region, at least one L7 loop motif region, and at least one 3F1 3′ flanking region.

In some embodiments, the molecular scaffold may comprise at least one 5F5 5′ flanking region, at least one L4 loop motif region, and at least one 3F4 3′ flanking region.

In some embodiments, the molecular scaffold may comprise at least one 5F6 5′ flanking region, at least one L4 loop motif region, and at least one 3F1 3′ flanking region.

in some embodiments, the molecular scaffold may comprise at least one 5F3 5′ flanking region, at least one L6 loop motif region, and at least one 3F1 3′ flanking region.

In some embodiments, the molecular scaffold may comprise at least one 5F7 5′ flanking region, at least one L4 loop motif region, and at least one 3F5 3′ flanking region.

In some embodiments, the molecular scaffold may comprise at least one 5F2 5′ flanking region, at least one L2 loop motif region, and at least one 3F2 3′ flanking region.

In some embodiments, the molecular scaffold may comprise at least one 5F2 5′ flanking region, at least one L1 loop motif region, and at least one 3F3 3′ flanking region.

In some embodiments, the molecular scaffold may comprise at least one 5F3 5′ flanking region, at least one L5 loop motif region, and at least one 3F4 3′ flanking region.

In some embodiments, the molecular scaffold may comprise at least one 5F1 5′ flanking region, at least one L1 loop motif region, and at least one 3F1 3′ flanking region,

In some embodiments, the molecular scaffold may comprise at least one 5F1 5′ flanking region, at least one L2 loop motif region, and at least one 3F1 3′ flanking region.

in some embodiments, the molecular scaffold may comprise at least one 5F1 5′ flanking region, at least one L1 loop motif region, and at least one 3F2 3′ flanking region.

In some embodiments, the molecular scaffold may comprise at least one 5F2 5′ flanking region, at least one L3 loop motif region, and at least one 3F3 3′ flanking region.

In some embodiments, the molecular scaffold may be a natural pri-miRNA scaffold. As a non-limiting example, the molecular scaffold may be a scaffold derived from the human miR155 scaffold.

in some embodiments, the molecular scaffold may comprise one or more linkers known in the art. The linkers may separate regions or one molecular scaffold from another. As a non-limiting example, the molecular scaffold may be polycistronic.

Modulatory Polynucleotide Comprising Molecular Scaffold and siRNA Molecule

In some embodiments, the modulatory polynucleotide may comprise 5′ and 3′ flanking regions, loop motif region, and nucleic acid sequences encoding sense sequence and antisense sequence as described in Tables 10 and 11. In Tables 10 and 11, the DNA sequence identifier for the passenger and guide strands are described as well as the 5′ and 3′ Flanking Regions and the Loop region (also referred to as the linker region). In Tables 10 and 11, the “miR” component of the name of the sequence does not necessarily correspond to the sequence numbering of miRNA genes (e.g., VOYHTmiR-102 is the name of the sequence and does not necessarily mean that miR-102 is part of the sequence).

TABLE 10
Modulatory Polynucleotide Sequence Region (5′ to 3′)

| Modulatory | 5′ Flanking to | | | | | |
|---|---|---|---|---|---|---|
| Polynucleotide | 3′ Flanking | 5′ Flanking | Passenger | Loop | Guide | 3′ Flanking |
| Construct Name | SEQ ID NO | SEQ ID NO | SEQ ID NO | SEQ ID NO | SEQ ID NO | SEQ ID NO |
| VOYHTmiR-102.214 | 1183 | 1161 | 1296 | 1168 | 1337 | 1178 |
| VOYHTmiR-104.214 | 1184 | 1161 | 1303 | 1168 | 1337 | 1178 |
| VOYHTmiR-109.214 | 1185 | 1161 | 1310 | 1169 | 1337 | 1178 |
| VOYHTmiR-114.214 | 1186 | 1161 | 1317 | 1168 | 1337 | 1179 |
| VOYHTmiR-116.214 | 1187 | 1161 | 1310 | 1168 | 1337 | 1179 |
| VOYHTmiR-127.214 | 1188 | 1162 | 1310 | 1170 | 1334 | 1180 |
| VOYHTmiR-102.218 | 1189 | 1161 | 1297 | 1168 | 1338 | 1178 |
| VOYHTmiR-104.218 | 1190 | 1161 | 1304 | 1168 | 1338 | 1178 |
| VOYHTmiR-109.218 | 1191 | 1161 | 1311 | 1169 | 1338 | 1178 |
| VOYHTmiR-114.218 | 1192 | 1161 | 1318 | 1168 | 1338 | 1179 |
| VOYHTmiR-116.218 | 1193 | 1161 | 1311 | 1168 | 1338 | 1179 |
| VOYHTmiR-127.218 | 1194 | 1162 | 1311 | 1170 | 1338 | 1180 |
| VOYHTmiR-102.219.o | 1195 | 1161 | 1280 | 1168 | 1333 | 1178 |
| VOYHTmiR-104.219.o | 1196 | 1161 | 1283 | 1168 | 1333 | 1178 |
| VOYHTmiR-109.219.o | 1197 | 1161 | 1280 | 1169 | 1333 | 1178 |
| VOYHTmiR-114.219 | 1198 | 1161 | 1286 | 1168 | 1333 | 1179 |
| VOYHTmiR-116.219.o | 1199 | 1161 | 1289 | 1168 | 1333 | 1179 |
| VOYHTmiR-127.219.o | 1200 | 1162 | 1280 | 1170 | 1333 | 1180 |
| VOYHTmiR-102.219.n | 1201 | 1161 | 1292 | 1168 | 1333 | 1178 |
| VOYHTmiR-104.219.n | 1202 | 1161 | 1293 | 1168 | 1333 | 1178 |
| VOYHTmiR-109.219.n | 1203 | 1161 | 1292 | 1169 | 1333 | 1178 |
| VOYHTmiR-116.219.n | 1204 | 1161 | 1294 | 1168 | 1333 | 1179 |
| VOYHTmiR-127.219.n | 1205 | 1162 | 1292 | 1170 | 1333 | 1180 |
| VOYHTmiR-102.257 | 1206 | 1161 | 1298 | 1168 | 1339 | 1178 |
| VOYHTmiR-104.257 | 1207 | 1161 | 1305 | 1168 | 1339 | 1178 |
| VOYHTmiR-109.257 | 1208 | 1161 | 1312 | 1169 | 1339 | 1178 |
| VOYHTmiR-114.257 | 1209 | 1161 | 1319 | 1168 | 1339 | 1179 |
| VOYHTmiR-116.257 | 1210 | 1161 | 1312 | 1168 | 1339 | 1179 |
| VOYHTmiR-127.257 | 1211 | 1162 | 1312 | 1170 | 1339 | 1180 |
| VOYHTmiR-102.894 | 1212 | 1161 | 1281 | 1168 | 1334 | 1178 |
| VOYHTmiR-104.894 | 1213 | 1161 | 1284 | 1168 | 1334 | 1178 |
| VOYHTmiR-109.894 | 1214 | 1161 | 1281 | 1169 | 1334 | 1178 |
| VOYHTmiR-114.894 | 1215 | 1161 | 1287 | 1168 | 1334 | 1179 |
| VOYHTmiR-116.894 | 1216 | 1161 | 1290 | 1168 | 1334 | 1179 |
| VOYHTmiR-127.894 | 1217 | 1162 | 1281 | 1170 | 1334 | 1180 |
| VOYHTmiR-102.907 | 1218 | 1161 | 1301 | 1168 | 1342 | 1178 |
| VOYHTmiR-104.907 | 1219 | 1161 | 1308 | 1168 | 1342 | 1178 |
| VOYHTmiR-109.907 | 1220 | 1161 | 1315 | 1169 | 1342 | 1178 |
| VOYHTmiR-114.907 | 1221 | 1161 | 1322 | 1168 | 1342 | 1179 |
| VOYHTmiR-116.907 | 1222 | 1161 | 1315 | 1168 | 1342 | 1179 |
| VOYHTmiR-127.907 | 1223 | 1162 | 1315 | 1170 | 1342 | 1180 |
| VOYHTmiR-102.372 | 1224 | 1161 | 1299 | 1168 | 1340 | 1178 |
| VOYHTmiR-104.372 | 1225 | 1161 | 1306 | 1168 | 1340 | 1178 |
| VOYHTmiR-109.372 | 1226 | 1161 | 1313 | 1169 | 1340 | 1178 |
| VOYHTmiR-114.372 | 1227 | 1161 | 1320 | 1168 | 1340 | 1179 |
| VOYHTmiR-116.372 | 1228 | 1161 | 1313 | 1168 | 1340 | 1179 |
| VOYHTmiR-127.372 | 1229 | 1162 | 1313 | 1170 | 1340 | 1180 |
| VOYHTmiR-102.425 | 1230 | 1161 | 1300 | 1168 | 1341 | 1178 |
| VOYHTmiR-104.425 | 1231 | 1161 | 1307 | 1168 | 1341 | 1178 |
| VOYHTmiR-109.425 | 1232 | 1161 | 1314 | 1169 | 1341 | 1178 |
| VOYHTmiR-114.425 | 1233 | 1161 | 1321 | 1168 | 1341 | 1179 |
| VOYHTmiR-116.425 | 1234 | 1161 | 1314 | 1168 | 1341 | 1179 |
| VOYHTmiR-127.425 | 1235 | 1162 | 1314 | 1170 | 1341 | 1180 |
| VOYHTmiR-102.032 | 1236 | 1161 | 1324 | 1168 | 1344 | 1178 |
| VOYHTmiR-104.032 | 1237 | 1161 | 1326 | 1168 | 1344 | 1178 |
| VOYHTmiR-109.032 | 1238 | 1161 | 1328 | 1169 | 1344 | 1178 |
| VOYHTmiR-114.032 | 1239 | 1161 | 1330 | 1168 | 1344 | 1179 |
| VOYHTmiR-116.032 | 1240 | 1161 | 1328 | 1168 | 1344 | 1179 |
| VOYHTmiR-127.032 | 1241 | 1162 | 1328 | 1170 | 1344 | 1180 |
| VOYHTmiR-102.020 | 1242 | 1161 | 1323 | 1168 | 1343 | 1178 |
| VOYHTmiR-104.020 | 1243 | 1161 | 1325 | 1168 | 1343 | 1178 |
| VOYHTmiR-109.020 | 1244 | 1161 | 1327 | 1169 | 1343 | 1178 |
| VOYHTmiR-114.020 | 1245 | 1161 | 1329 | 1168 | 1343 | 1179 |
| VOYHTmiR-116.020 | 1246 | 1161 | 1327 | 1168 | 1343 | 1179 |
| VOYHTmiR-127.020 | 1247 | 1162 | 1327 | 1170 | 1343 | 1180 |
| VOYHTmiR-102.016 | 1248 | 1161 | 1295 | 1168 | 1336 | 1178 |
| VOYHTmiR-104.016 | 1249 | 1161 | 1302 | 1168 | 1336 | 1178 |
| VOYHTmiR-109.016 | 1250 | 1161 | 1309 | 1169 | 1336 | 1178 |
| VOYHTmiR-114.016 | 1251 | 1161 | 1316 | 1168 | 1336 | 1179 |
| VOYHTmiR-116.016 | 1252 | 1161 | 1309 | 1168 | 1336 | 1179 |
| VOYHTmiR-127.016 | 1253 | 1162 | 1309 | 1170 | 1336 | 1180 |
| VOYHTmiR-102.579 | 1254 | 1161 | 1282 | 1168 | 1335 | 1178 |
| VOYHTmiR-104.579 | 1255 | 1161 | 1285 | 1168 | 1335 | 1178 |
| VOYHTmiR-109.579 | 1256 | 1161 | 1282 | 1169 | 1335 | 1178 |
| VOYHTmiR-114.579 | 1257 | 1161 | 1288 | 1168 | 1335 | 1179 |
| VOYHTmiR-116.579 | 1258 | 1161 | 1291 | 1168 | 1335 | 1179 |
| VOYHTmiR-127.579 | 1259 | 1162 | 1282 | 1170 | 1335 | 1180 |
| VOYHTmiR-104.579.1 | 1260 | 1161 | 1331 | 1171 | 1335 | 1178 |
| VOYHTmiR-104.579.2 | 1261 | 1163 | 1331 | 1171 | 1335 | 1178 |
| VOYHTmiR-104.579.3 | 1262 | 1163 | 1331 | 1172 | 1335 | 1178 |
| VOYHTmiR-104.579.4 | 1263 | 1164 | 1331 | 1171 | 1335 | 1181 |
| VOYHTmiR-104.579.6 | 1264 | 1165 | 1331 | 1171 | 1335 | 1181 |
| VOYHTmiR-104.579.7 | 1265 | 1166 | 1331 | 1171 | 1345 | 1178 |
| VOYHTmiR-104.579.8 | 1266 | 1163 | 1332 | 1173 | 1335 | 1178 |
| VOYHTmiR-104.579.9 | 1267 | 1167 | 1331 | 1171 | 1335 | 1182 |
| VOYHTmiR-102.020 | 1268 | 1161 | 1323 | 1168 | 1343 | 1178 |
| VOYHTmiR-102.032 | 1269 | 1161 | 1324 | 1168 | 1344 | 1178 |
| VOYHTmiR-104.020 | 1270 | 1161 | 1325 | 1168 | 1343 | 1178 |
| VOYHTmiR-104.032 | 1271 | 1161 | 1326 | 1168 | 1344 | 1178 |
| VOYHTmiR-109.020 | 1272 | 1161 | 1327 | 1169 | 1343 | 1178 |
| VOYHTmiR-109.032 | 1273 | 1161 | 1328 | 1169 | 1344 | 1178 |
| VOYHTmiR-114.020 | 1274 | 1161 | 1329 | 1168 | 1343 | 1179 |
| VOYHTmiR-114.032 | 1275 | 1161 | 1330 | 1168 | 1344 | 1179 |
| VOYHTmiR-116.020 | 1276 | 1161 | 1327 | 1168 | 1343 | 1179 |
| VOYHTmiR-116.032 | 1277 | 1161 | 1328 | 1168 | 1344 | 1179 |
| VOYHTmiR-127.020 | 1278 | 1162 | 1327 | 1170 | 1343 | 1180 |
| VOYHTmiR-127.032 | 1279 | 1162 | 1328 | 1170 | 1344 | 1180 |

TABLE 11
Modulatory Polynucleotide Sequence Region (5′ to 3′)

| | 5′ Flanking to | | | | | |
|---|---|---|---|---|---|---|
| | 3′ Flanking | 5′ Flanking | Guide | Loop | Passenger | 3′ Flanking |
| Name | SEQ ID NO | SEQ ID NO | SEQ ID NO | SEQ ID NO | SEQ ID NO | SEQ ID NO |
| VOYHTmiR-104.579.5 | 1346 | 1163 | 1350 | 1174 | 1348 | 1178 |
| VOYHTmiR-104.579.10 | 1347 | 1167 | 1351 | 1175 | 1349 | 1182 |

### AAV Particles Comprising Modulator Polynucleotides

In some embodiments, the AAV particle comprises a viral genome with a payload region comprising a modulatory polynucleotide sequence. In such an embodiment, a viral genome encoding more than one polypeptide may be replicated and packaged into a viral particle. A target cell transduced with a viral particle comprising a modulatory polynucleotide may express the encoded sense and/or antisense sequences in a single cell.

In some embodiments, the AAV particles are useful in the field of medicine for the treatment, prophylaxis, palliation or amelioration of neurological diseases and/or disorders.

Non-limiting examples of ITR to ITR sequences of AAV particles comprising a viral genome with a payload region comprising a modulatory polynucleotide sequence are described in Table 12.

TABLE 12
ITR to ITR Sequences of AAV Particles
comprising Modulatory Polynucleotides

| ITR to ITR | ITR to ITR | Modulatory Polynucleotide |
|---|---|---|
| Construct Name | SEQ ID NO | SEQ ID NO |
| VOYHT1 | 1352 | 1262 |
| VOYHT2 | 1353 | 1262 |
| VOYHT3 | 1354 | 1250 |
| VOYHT4 | 1355 | 1347 |
| VOYHT5 | 1356 | 1262 |
| VOYHT6 | 1357 | 1262 |
| VOYHT7 | 1358 | 1250 |
| VOYHT8 | 1359 | 1347 |
| VOYHT9 | 1360 | 1262 |
| VOYHT10 | 1361 | 1262 |
| VOYHT11 | 1362 | 1250 |
| VOYHT12 | 1363 | 1347 |
| VOYHT13 | 1364 | 1262 |
| VOYHT14 | 1365 | 1249 |
| VOYHT15 | 1366 | 1259 |
| VOYHT16 | 1367 | 1249 |
| VOYHT17 | 1368 | 1262 |
| VOYHT18 | 1369 | 1262 |
| VOYHT19 | 1370 | 1262 |
| VOYHT20 | 1371 | 1262 |
| VOYHT21 | 1372 | 1262 |
| VOYHT22 | 1373 | 1262 |
| VOYHT23 | 1374 | 1262 |
| VOYHT24 | 1375 | 1262 |
| VOYHT25 | 1376 | 1249 |
| VOYHT26 | 1377 | 1262 |
| VOYHT27 | 1378 | 1259 |
| VOYHT28 | 1379 | 1249 |
| VOYHT35 | 1388 | 1255 |
| VOYHT36 | 1426 | 1248 |
| VOYHT37 | 1427 | 1231 |
| VOYHT38 | 1428 | 1219 |
| VOYHT39 | 1429 | 1207 |
| VOYHT40 | 1430 | 1250 |
| VOYHT41 | 1431 | 1251 |
| VOYHT42 | 1432 | 1252 |
| VOYHT43 | 1433 | 1253 |
| VOYHT44 | 1434 | 1194 |
| VOYHT45 | 1435 | 1223 |
| VOYHT46 | 1436 | 1211 |
| VOYHT47 | 1437 | 1262 |
| VOYHT48 | 1438 | 1347 |

In some embodiments, the AAV particle comprises a viral genome which comprises a sequence which has a percent identity to any of SEQ ID NOs: 1352-1379, 1388, and 1426-1438. The viral genome may have 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99% or 100% identity to any of SEQ NOs: 1352-1379, 1388, and 1426-1438. The viral genome may have 1-10%, 10-20%, 30-40%, 50-60%, 50-70%, 50-80%, 50-90%, 50-99%, 50-100%, 60-70%, 60-80%, 60-90%, 60-99%, 60-100%, 70-80%, 70-90%, 70-99%, 70-100%, 80-85%, 80-90%, 80-95%, 80-99%, 80-100%, 90-95%, 90-99%, or 90-100% to any of SEQ ID NOs: 1352-1379, 1388, and 1426-1438. As a non-limiting example, the viral genome comprises a sequence which as 80% identity to any of SEQ ID NO: 1352-1379, 1388, and 1426-1438. As another non-limiting example, the viral genome comprises a sequence which as 85% identity to any of SEQ ID NO: 1352-1379, 1388, and 1426-1438. As another non-limiting example, the viral genome comprises a sequence which as 90% identity to any of SEQ ID NO: 1352-1379, 1388, and 1426-1438. As another non-limiting example, the viral genome comprises a sequence which as 95% identity to any of SEQ ID NO: 1352-1379, 1388, and 1426-1438. As another non-limiting example, the viral genome comprises a sequence which as 99% identity to any of SEQ ID NO: 1352-1379, 1388, and 1426-1438.

In some embodiments, the AAV particle comprises a viral genome which comprises a sequence corresponding to SEQ ID NOs: 1352, or variants having at least 95% identity thereof. The AAV particle may comprise an AAV1 serotype.

In some embodiments, the AAV particles comprising modulatory polynucleotide sequence which comprises a nucleic acid sequence encoding at least one siRNA molecule may he introduced into mammalian cells.

Where the AAV particle payload region comprises a modulatory polynucleotide, the modulatory polynucleotide may comprise sense and/or antisense sequences to knock down a target gene. The AAV viral genomes encoding modulatory polynucleotides described herein may be useful in the fields of human disease, viruses, infections veterinary applications and a variety of in vivo and in vitro settings.

In some embodiments, the AAV particle genome may comprise at least one inverted terminal repeat (ITR) region, The ITR region(s) may, independently, have a length such as, but not limited to, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, and 175 nucleotides. The length of the ITR region for the viral genome may be 75-80, 75-85, 75-100, 80-85, 80-90, 80-105, 85-90, 85-95, 85-110, 90-95, 90-100, 90-115, 95-100, 95-105, 95-120, 100-105, 100-110, 100-125, 105-110, 105-115, 105-130, 110-115, 110-120, 110-135, 115-120, 115-125, 115-140, 120-125, 120-130, 120-145, 125-130, 125-135, 125-150, 130-135, 130-140, 130-155, 135-140, 135-145, 135-160, 140-145, 140-150, 140-165, 145-150, 145-155, 145-170, 150-155, 150-160, 150-175, 155-160, 155-165, 160-165, 160-170, 165-170, 165-175, and 170-175 nucleotides. As a non-limiting example, the viral genome comprises an ITR that is about 105 nucleotides in length. As a non-limiting example, the viral genome comprises an ITR that is about 141 nucleotides in length. As a non-limiting example, the viral genome comprises an ITR that is about 130 nucleotides in length.

In some embodiments, the AAV particle viral genome may comprise two inverted terminal repeat (ITR) regions. Each of the ITR regions may independently have a length such as, but not limited to, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, and 175 nucleotides. The length of the ITR regions for the viral genome may be 75-80, 75-85, 75-100, 80-85, 80-90, 80-105, 85-90, 85-95, 85-110, 90-95, 90-100, 90-115, 95-100, 95-105, 95-120, 100-105, 100-110, 100-125, 105-110, 105-115, 105-130, 110-115, 110-120, 110-135, 115-120, 115-125, 115-140, 120-125, 120-130, 120-145, 125-130, 125-135, 125-150, 130-135, 130-140, 130-155, 135-140, 135-145, 135-160, 140-145, 140-150, 140-165, 145-150, 145-155, 145-170, 150-155, 150-160, 150-175, 155-160, 155-165, 160-165, 160-170, 165-170, 165-175, and 170-175 nucleotides. As a non-limiting example, the viral genome comprises an ITR that is about 105 nucleotides in length and 141 nucleotides in length. As a non-limiting example, the viral genome comprises an ITR that is about 105 nucleotides in length and 130 nucleotides in length. As a non-limiting example, the viral genome comprises an IIR that is about 130 nucleotides in length and 141 nucleotides in length.

In some embodiments, the AAV particle viral genome may comprise at least one sequence region as described in Tables 13-20. The regions may be located before or after any of the other sequence regions described herein.

In some embodiments, the AAV particle viral genome comprises at least one inverted terminal repeat (ITR) sequence region. Non-limiting examples of ITR sequence regions are described in Table 13.

TABLE 13
Inverted Terminal Repeat (ITR) Sequence Regions

| Sequence Region Name | SEQ ID NO |
|---|---|
| ITR1 | 1380 |
| ITR2 | 1381 |
| ITR3 | 1382 |
| ITR4 | 1383 |

In some embodiments, the AAV particle viral genome comprises two ITR sequence regions. In some embodiments, the ITR sequence regions are the ITRI sequence region and the ITR3 sequence region. In some embodiments, the ITR sequence regions are the ITRI sequence region and the ITR4 sequence region. In some embodiments, the ITR sequence regions are the ITR2 sequence region and the ITR3 sequence region. In some embodiments, the ITR sequence regions are the ITR2 sequence region and the ITR4 sequence region.

In some embodiments, the AAV particle viral genome may comprise at least one multiple cloning site (MCS) sequence region. The MCS region(s) may, independently, have a length such as, but not limited to, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, and 150 nucleotides. The length of the MCS region for the viral genome may be 2-10, 5-10, 5-15, 10-20, 10-30, 10-40, 15-20, 15-25, 20-30, 20-40, 20-50, 25-30, 25-35, 30-40, 30-50, 30-60, 35-40, 35-45, 40-50, 40-60, 40-70, 45-50, 45-55, 50-60, 50-70, 50-80, 55-60, 55-65, 60-70, 60-80, 60-90, 65-70, 65-75, 70-80, 70-90, 70-100, 75-80, 75-85, 80-90, 80-100, 80-110, 85-90, 85-95, 90-100, 90-110, 90-120, 95-100, 95-105, 100-110, 100-120, 100-130, 105-110, 105-115, 110-120, 110-130, 110-140, 115-120, 115-125, 120-130, 120-140, 120-150, 125-130, 125-135, 130-140, 130-150, 135-140, 135-145, 140-150, and 145-150 nucleotides. As a non-limiting example, the viral genome comprises a MCS region that is about 5 nucleotides in length. As a non-limiting example, the viral genome comprises a MCS region that is about 10 nucleotides in length. As a non-limiting example, the viral genome comprises a MCS region that is about 14 nucleotides in length. As a non-limiting example, the viral genome comprises a MCS region that is about 18 nucleotides in length. As a non-limiting example, the viral genome comprises a MCS region that is about 73 nucleotides in length. As a non-limiting example, the viral genome comprises a MCS region that is about 121 nucleotides in length.

In some embodiments, the AAV particle viral genome comprises at least one multiple cloning site (MCS) sequence regions. Non-limiting examples of MCS sequence regions are described in Table 14.

TABLE 14
Multiple Cloning Site (MCS) Sequence Regions

| | SEQ ID NO or |
|---|---|
| Sequence Region Name | Sequence |
| MCS1 | 1384 |
| MCS2 | 1385 |
| MCS3 | 1386 |
| MCS4 | 1387 |
| MCS5 | TCGAG |
| MCS6 | 1389 |

In some embodiments, the AAV particle viral genome comprises one MCS sequence region. In some embodiments, the MCS sequence region is the MCS1 sequence region. In some embodiments, the MCS sequence region is the MCS2 sequence region. In some embodiments, the MCS sequence region is the MCS3 sequence region. In some embodiments, the MCS sequence region is the MCS4 sequence region. In some embodiments, the MCS sequence region is the MCS5 sequence region. In some embodiments, the MCS sequence region is the MCS6 sequence region.

In some embodiments, the AAV particle viral genome comprises two MCS sequence regions. In some embodiments, the two MCS sequence regions are the MCS1 sequence region and the MCS2 sequence region. In some embodiments, the two MCS sequence regions are the MCSI sequence region and the MCS3 sequence region. In some embodiments, the two MCS sequence regions are the MCS1 sequence region and the MCS4 sequence region. In some embodiments, the two MCS sequence regions are the MCS1 sequence region and the MCS5 sequence region. In some embodiments, the two MCS sequence regions are the MCS1 sequence region and the MCS6 sequence region. In some embodiments, the two MCS sequence regions are the MCS2 sequence region and the MCS3 sequence region. In some embodiments, the two MCS sequence regions are the MCS2 sequence region and the MCS4 sequence region. In some embodiments, the two MCS sequence regions are the MCS2 sequence region and the MCS5 sequence region. In some embodiments, the two MCS sequence regions are the MCS2 sequence region and the MCS6 sequence region. In some embodiments, the two MCS sequence regions are the MCS3 sequence region and the MCS4 sequence region. In some embodiments, the two MCS sequence regions are the MCS3 sequence region and the MCS5 sequence region. In some embodiments, the two MCS sequence regions are the MCS3 sequence region and the MCS6 sequence region. In some embodiments, the two MCS sequence regions are the MCS4 sequence region and the MCS5 sequence region. In some embodiments, the two MCS sequence regions are the MCS4 sequence region and the MCS6 sequence region. In some embodiments, the two MCS sequence regions are the MCS5 sequence region and the MCS6 sequence region.

In some embodiments, the AAV particle viral genome comprises two or more MCS sequence regions.

In some embodiments, the AAV particle viral genome comprises three MCS sequence regions. In some embodiments, the three MCS sequence regions are the MCS1 sequence region, the MCS2 sequence region, and the MCS3 sequence region. In some embodiments, the three MCS sequence regions are the MCS1 sequence region, the MCS2 sequence region, and the MCS4 sequence region. In some embodiments, the three MCS sequence regions are the MCS1 sequence region, the MCS2 sequence region, and the MCS5 sequence region. In some embodiments, the three MCS sequence regions are the MCSI sequence region, the MCS2 sequence region, and the MCS6 sequence region. In some embodiments, the three MCS sequence regions are the MCS 1 sequence region, the MCS3 sequence region, and the MCS4 sequence region. In some embodiments, the three MCS sequence regions are the MCS1 sequence region, the MCS3 sequence region, and the MCS5 sequence region. In some embodiments, the three MCS sequence regions are the MCS1 sequence region, the MCS3 sequence region, and the MCS6 sequence region. In some embodiments, the three MCS sequence regions are the MCS1 sequence region, the MCS4 sequence region, and the MCS5 sequence region. In some embodiments, the three MCS sequence regions are the MCS sequence region, the MCS4 sequence region, and the MCS6 sequence region. In some embodiments, the three MCS sequence regions are the MCS1 sequence region, the MCS5 sequence region, and the MCS6 sequence region. In some embodiments, the three MCS sequence regions are the MCS2 sequence region, the MCS3 sequence region, and the MCS4 sequence region. In some embodiments, the three MCS sequence regions are the MCS2 sequence region, the MCS3 sequence region, and the MCS5 sequence region. In some embodiments, the three MCS sequence regions are the MCS2 sequence region, the MCS3 sequence region, and the MCS6 sequence region. in some embodiments, the three MCS sequence regions are the MCS2 sequence region, the MCS4 sequence region, and the MCS5 sequence region. In some embodiments, the three MCS sequence regions are the MCS2 sequence region, the MCS4 sequence region, and the MCS6 sequence region. In some embodiments, the three MCS sequence regions are the MCS2 sequence region, the MCSS sequence region, and the MCS6 sequence region. In some embodiments, the three MCS sequence regions are the MCS3 sequence region, the MCS4 sequence region, and the MCS5 sequence region. In some embodiments, the three MCS sequence regions are the MCS3 sequence region, the MCS4 sequence region, and the MCS6 sequence region. In some embodiments, the three MCS sequence regions are the MCS3 sequence region, the MCS5 sequence region, and the MCS6 sequence region. In some embodiments, the three MCS sequence regions are the MCS4 sequence region, the MCSS sequence region, and the MCS6 sequence region.

In some embodiments, the AAV particle viral genome may comprise at least one filler sequence region. The filler region(s) may, independently, have a length such as, but not limited to, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, 1009 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1058, 1059, 1060, 1061, 1062, 1063, 1064, 1065, 1066, 1067, 1068, 1069, 1070, 1071, 1072, 1073, 1074, 1075, 1076, 1077, 1078, 1079, 1080, 1081, 1082, 1083, 1084, 1085, 1086, 1087, 1088, 1089, 1090, 1091, 1092, 1093, 1094, 1095, 1096, 1097, 1098, 1099, 1100, 1101, 1102, 1103, 1104, 1105, 1106, 1107, 1108, 1109, 1110, 1111, 1112, 1113, 1114, 1115, 1116, 1117, 1118, 1119, 1120, 1121, 1122, 1123, 1124, 1125, 1126, 1127, 1128, 1129, 1130, 1131, 1132, 1133, 1134, 1135, 1136, 1137, 1138, 1139, 1140, 1141, 1142, 1143, 1144, 1145, 1146, 1147, 1148, 1149, 1150, 1151, 1152, 1153, 1154, 1155, 1156, 1157, 1158, 1159, 1160, 1161, 1162, 1163, 1164, 1165, 1166, 1167, 1168, 1169, 1170, 1171, 1172, 1173, 1174, 1175, 1176, 1177, 1178, 1179, 1180, 1181, 1182, 1183, 1184, 1185, 1186, 1187, 1188, 1189, 1190, 1191, 1192, 1193, 1194, 1195, 1196, 1197, 1198, 1199, 1200, 1201, 1202, 1203, 1204, 1205, 1206, 1207, 1208, 1209, 1210, 1211, 1212, 1213, 1214, 1215, 1216, 1217, 1218, 1219, 1220, 1221, 1222, 1223, 1224, 1225, 1226, 1227, 1228, 1229, 1230, 1231, 1232, 1233, 1234, 1235, 1236, 1237, 1238, 1239, 1240, 1241, 1242, 1243, 1244, 1245, 1246, 1247, 1248, 1249, 1250, 1251, 1252, 1253, 1254, 1255, 1256, 1257, 1258, 1259, 1260, 1261, 1262, 1263, 1264, 1265, 1266, 1267, 1268, 1269, 1270, 1271, 1272, 1273, 1274, 1275, 1276, 1277, 1278, 1279, 1280, 1281, 1282, 1283, 1284, 1285, 1286, 1287, 1288, 1289, 1290, 1291, 1292, 1293, 1294, 1295, 1296, 1297, 1298, 1299, 1300, 1301, 1302, 1303, 1304, 1305, 1306, 1307, 1308, 1309, 1310, 1311, 1312, 1313, 1314, 1315, 1316, 1317, 1318, 1319, 1320, 1321, 1322, 1323, 1324, 1325, 1326, 1327, 1328, 1329, 1330, 1331, 1332, 1333, 1334, 1335, 1336, 1337, 1338, 1339, 1340, 1341, 1342, 1343, 1344, 1345, 1346, 1347, 1348, 1349, 1350, 1351, 1352, 1353, 1354, 1355, 1356, 1357, 1358, 1359, 1360, 1361, 1362, 1363, 1364, 1365, 1366, 1367, 1368, 1369, 1370, 1371, 1372, 1373, 1374, 1375, 1376, 1377, 1378, 1379, 1380, 1381, 1382, 1383, 1384, 1385, 1386, 1387, 1388, 1389, 1390, 1391, 1392, 1393, 1394, 1395, 1396, 1397, 1398, 1399, 1400, 1401, 1402, 1403, 1404, 1405, 1406, 1407, 1408, 1409, 1410, 1411, 1412, 1413, 1414, 1415, 1416, 1417, 1418, 1419, 1420, 1421, 1422, 1423, 1424, 1425, 1426, 1427, 1428, 1429, 1430, 1431, 1432, 1433, 1434, 1435, 1436, 1437, 1438, 1439, 1440, 1441, 1442, 1443, 1444, 1445, 1446, 1447, 1448, 1449, 1450, 1451, 1452, 1453, 1454, 1455, 1456, 1457, 1458, 1459, 1460, 1461, 1462, 1463, 1464, 1465, 1466, 1467, 1468, 1469, 1470, 1471, 1472, 1473, 1474, 1475, 1476, 1477, 1478, 1479, 1480, 1481, 1482, 1483, 1484, 1485, 1486, 1487, 1488, 1489, 1490, 1491, 1492, 1493, 1494, 1495, 1496, 1497, 1498, 1499, 1500, 1501, 1502, 1503, 1504, 1505, 1506, 1507, 1508, 1509, 1510, 1511, 1512, 1513, 1514, 1515, 1516, 1517, 1518, 1519, 1520, 1521, 1522, 1523, 1524, 1525, 1526, 1527, 1528, 1529, 1530, 1531, 1532, 1533, 1534, 1535, 1536, 1537, 1538, 1539, 1540, 1541, 1542, 1543, 1544, 1545, 1546, 1547, 1548, 1549, 1550, 1551, 1552, 1553, 1554, 1555, 1556, 1557, 1558, 1559, 1560, 1561, 1562, 1563, 1564, 1565, 1566, 1567, 1568, 1569, 1570, 1571, 1572, 1573, 1574, 1575, 1576, 1577, 1578, 1579, 1580, 1581, 1582, 1583, 1584, 1585, 1586, 1587, 1588, 1589, 1590, 1591, 1592, 1593, 1594, 1595, 1596, 1597, 1598, 1599, 1600, 1601, 1602, 1603, 1604, 1605, 1606, 1607, 1608, 1609, 1610, 1611, 1612, 1613, 1614, 1615, 1616, 1617, 1618, 1619, 1620, 1621, 1622, 1623, 1624, 1625, 1626, 1627, 1628, 1629, 1630, 1631, 1632, 1633, 1634, 1635, 1636, 1637, 1638, 1639, 1640, 1641, 1642, 1643, 1644, 1645, 1646, 1647, 1648, 1649, 1650, 1651, 1652, 1653, 1654, 1655, 1656, 1657, 1658, 1659, 1660, 1661, 1662, 1663, 1664, 1665, 1666, 1667, 1668, 1669, 1670, 1671, 1672, 1673, 1674, 1675, 1676, 1677, 1678, 1679, 1680, 1681, 1682, 1683, 1684, 1685, 1686, 1687, 1688, 1689, 1690, 1691, 1692, 1693, 1694, 1695, 1696, 1697, 1698, 1699, 1700, 1701, 1702, 1703, 1704, 1705, 1706, 1707, 1708, 1709, 1710, 1711, 1712, 1713, 1714, 1715, 1716, 1717, 1718, 1719, 1720, 1721, 1722, 1723, 1724, 1725, 1726, 1727, 1728, 1729, 1730, 1731, 1732, 1733, 1734, 1735, 1736, 1737, 1738, 1739, 1740, 1741, 1742, 1743, 1744, 1745, 1746, 1747, 1748, 1749, 1750, 1751, 1752, 1753, 1754, 1755, 1756, 1757, 1758, 1759, 1760, 1761, 1762, 1763, 1764, 1765, 1766, 1767, 1768, 1769, 1770, 1771, 1772, 1773, 1774, 1775, 1776, 1777, 1778, 1779, 1780, 1781, 1782, 1783, 1784, 1785, 1786, 1787, 1788, 1789, 1790, 1791, 1792, 1793, 1794, 1795, 1796, 1797, 1798, 1799, 1800, 1801, 1802, 1803, 1804, 1805, 1806, 1807, 1808, 1809, 1810, 1811, 1812, 1813, 1814, 1815, 1816, 1817, 1818, 1819, 1820, 1821, 1822, 1823, 1824, 1825, 1826, 1827, 1828, 1829, 1830, 1831, 1832, 1833, 1834, 1835, 1836, 1837, 1838, 1839, 1840, 1841, 1842, 1843, 1844, 1845, 1846, 1847, 1848, 1849, 1850, 1851, 1852, 1853, 1854, 1855, 1856, 1857, 1858, 1859, 1860, 1861, 1862, 1863, 1864, 1865, 1866, 1867, 1868, 1869, 1870, 1871, 1872, 1873, 1874, 1875, 1876, 1877, 1878, 1879, 1880, 1881, 1882, 1883, 1884, 1885, 1886, 1887, 1888, 1889, 1890, 1891, 1892, 1893, 1894, 1895, 1896, 1897, 1898, 1899, 1900, 1901, 1902, 1903, 1904, 1905, 1906, 1907, 1908, 1909, 1910, 1911, 1912, 1913, 1914, 1915, 1916, 1917, 1918, 1919, 1920, 1921, 1922, 1923, 1924, 1925, 1926, 1927, 1928, 1929, 1930, 1931, 1932, 1933, 1934, 1935, 1936, 1937, 1938, 1939, 1940, 1941, 1942, 1943, 1944, 1945, 1946, 1947, 1948, 1949, 1950, 1951, 1952, 1953, 1954, 1955, 1956, 1957, 1958, 1959, 1960, 1961, 1962, 1963, 1964, 1965, 1966, 1967, 1968, 1969, 1970, 1971, 1972, 1973, 1974, 1975, 1976, 1977, 1978, 1979, 1980, 1981, 1982, 1983, 1984, 1985, 1986, 1987, 1988, 1989, 1990, 1991, 1992, 1993, 1994, 1995, 1996, 1997, 1998, 1999, 2000, 2001, 2002, 2003, 2004, 2005, 2006, 2007, 2008, 2009, 2010, 2011, 2012, 2013, 2014, 2015, 2016, 2017, 2018, 2019, 2020, 2021, 2022, 2023, 2024, 2025, 2026, 2027, 2028, 2029, 2030, 2031, 2032, 2033, 2034, 2035, 2036, 2037, 2038, 2039, 2040, 2041, 2042, 2043, 2044, 2045, 2046, 2047, 2048, 2049, 2050, 2051, 2052, 2053, 2054, 2055, 2056, 2057, 2058, 2059, 2060, 2061, 2062, 2063, 2064, 2065, 2066, 2067, 2068, 2069, 2070, 2071, 2072, 2073, 2074, 2075, 2076, 2077, 2078, 2079, 2080, 2081, 2082, 2083, 2084, 2085, 2086, 2087, 2088, 2089, 2090, 2091, 2092, 2093, 2094, 2095, 2096, 2097, 2098, 2099, 2100, 2101, 2102, 2103, 2104, 2105, 2106, 2107, 2108, 2109, 2110, 2111, 2112, 2113, 2114, 2115, 2116, 2117, 2118, 2119, 2120, 2121, 2122, 2123, 2124, 2125, 2126, 2127, 2128, 2129, 2130, 2131, 2132, 2133, 2134, 2135, 2136, 2137, 2138, 2139, 2140, 2141, 2142, 2143, 2144, 2145, 2146, 2147, 2148, 2149, 2150, 2151, 2152, 2153, 2154, 2155, 2156, 2157, 2158, 2159, 2160, 2161, 2162, 2163, 2164, 2165, 2166, 2167, 2168, 2169, 2170, 2171, 2172, 2173, 2174, 2175, 2176, 2177, 2178, 2179, 2180, 2181, 2182, 2183, 2184, 2185, 2186, 2187, 2188, 2189, 2190, 2191, 2192, 2193, 2194, 2195, 2196, 2197, 2198, 2199, 2200, 2201, 2202, 2203, 2204, 2205, 2206, 2207, 2208, 2209, 2210, 2211, 2212, 2213, 2214, 2215, 2216, 2217, 2218, 2219, 2220, 2221, 2222, 2223, 2224, 2225, 2226, 2227, 2228, 2229, 2230, 2231, 2232, 2233, 2234, 2235, 2236, 2237, 2238, 2239, 2240, 2241, 2242, 2243, 2244, 2245, 2246, 2247, 2248, 2249, 2250, 2251, 2252, 2253, 2254, 2255, 2256, 2257, 2258, 2259, 2260, 2261, 2262, 2263, 2264, 2265, 2266, 2267, 2268, 2269, 2270, 2271, 2272, 2273, 2274, 2275, 2276, 2277, 2278, 2279, 2280, 2281, 2282, 2283, 2284, 2285, 2286, 2287, 2288, 2289, 2290, 2291, 2292, 2293, 2294, 2295, 2296, 2297, 2298, 2299, 2300, 2301, 2302, 2303, 2304, 2305, 2306, 2307, 2308, 2309, 2310, 2311, 2312, 2313, 2314, 2315, 2316, 2317, 2318, 2319, 2320, 2321, 2322, 2323, 2324, 2325, 2326, 2327, 2328, 2329, 2330, 2331, 2332, 2333, 2334, 2335, 2336, 2337, 2338, 2339, 2340, 2341, 2342, 2343, 2344, 2345, 2346, 2347, 2348, 2349, 2350, 2351, 2352, 2353, 2354, 2355, 2356, 2357, 2358, 2359, 2360, 2361, 2362, 2363, 2364, 2365, 2366, 2367, 2368, 2369, 2370, 2371, 2372, 2373, 2374, 2375, 2376, 2377, 2378, 2379, 2380, 2381, 2382, 2383, 2384, 2385, 2386, 2387, 2388, 2389, 2390, 2391, 2392, 2393, 2394, 2395, 2396, 2397, 2398, 2399, 2400, 2401, 2402, 2403, 2404, 2405, 2406, 2407, 2408, 2409, 2410, 2411, 2412, 2413, 2414, 2415, 2416, 2417, 2418, 2419, 2420, 2421, 2422, 2423, 2424, 2425, 2426, 2427, 2428, 2429, 2430, 2431, 2432, 2433, 2434, 2435, 2436, 2437, 2438, 2439, 2440, 2441, 2442, 2443, 2444, 2445, 2446, 2447, 2448, 2449, 2450, 2451, 2452, 2453, 2454, 2455, 2456, 2457, 2458, 2459, 2460, 2461, 2462, 2463, 2464, 2465, 2466, 2467, 2468, 2469, 2470, 2471, 2472, 2473, 2474, 2475, 2476, 2477, 2478, 2479, 2480, 2481, 2482, 2483, 2484, 2485, 2486, 2487, 2488, 2489, 2490, 2491, 2492, 2493, 2494, 2495, 2496, 2497, 2498, 2499, 2500, 2501, 2502, 2503, 2504, 2505, 2506, 2507, 2508, 2509, 2510, 2511, 2512, 2513, 2514, 2515, 2516, 2517, 2518, 2519, 2520, 2521, 2522, 2523, 2524, 2525, 2526, 2527, 2528, 2529, 2530, 2531, 2532, 2533, 2534, 2535, 2536, 2537, 2538, 2539, 2540, 2541, 2542, 2543, 2544, 2545, 2546, 2547, 2548, 2549, 2550, 2551, 2552, 2553, 2554, 2555, 2556, 2557, 2558, 2559, 2560, 2561, 2562, 2563, 2564, 2565, 2566, 2567, 2568, 2569, 2570, 2571, 2572, 2573, 2574, 2575, 2576, 2577, 2578, 2579, 2580, 2581, 2582, 2583, 2584, 2585, 2586, 2587, 2588, 2589, 2590, 2591, 2592, 2593, 2594, 2595, 2596, 2597, 2598, 2599, 2600, 2601, 2602, 2603, 2604, 2605, 2606, 2607, 2608, 2609, 2610, 2611, 2612, 2613, 2614, 2615, 2616, 2617, 2618, 2619, 2620, 2621, 2622, 2623, 2624, 2625, 2626, 2627, 2628, 2629, 2630, 2631, 2632, 2633, 2634, 2635, 2636, 2637, 2638, 2639, 2640, 2641, 2642, 2643, 2644, 2645, 2646, 2647, 2648, 2649, 2650, 2651, 2652, 2653, 2654, 2655, 2656, 2657, 2658, 2659, 2660, 2661, 2662, 2663, 2664, 2665, 2666, 2667, 2668, 2669, 2670, 2671, 2672, 2673, 2674, 2675, 2676, 2677, 2678, 2679, 2680, 2681, 2682, 2683, 2684, 2685, 2686, 2687, 2688, 2689, 2690, 2691, 2692, 2693, 2694, 2695, 2696, 2697, 2698, 2699, 2700, 2701, 2702, 2703, 2704, 2705, 2706, 2707, 2708, 2709, 2710, 2711, 2712, 2713, 2714, 2715, 2716, 2717, 2718, 2719, 2720, 2721, 2722, 2723, 2724, 2725, 2726, 2727, 2728, 2729, 2730, 2731, 2732, 2733, 2734, 2735, 2736, 2737, 2738, 2739, 2740, 2741, 2742, 2743, 2744, 2745, 2746, 2747, 2748, 2749, 2750, 2751, 2752, 2753, 2754, 2755, 2756, 2757, 2758, 2759, 2760, 2761, 2762, 2763, 2764, 2765, 2766, 2767, 2768, 2769, 2770, 2771, 2772, 2773, 2774, 2775, 2776, 2777, 2778, 2779, 2780, 2781, 2782, 2783, 2784, 2785, 2786, 2787, 2788, 2789, 2790, 2791, 2792, 2793, 2794, 2795, 2796, 2797, 2798, 2799, 2800, 2801, 2802, 2803, 2804, 2805, 2806, 2807, 2808, 2809, 2810, 2811, 2812, 2813, 2814, 2815, 2816, 2817, 2818, 2819, 2820, 2821, 2822, 2823, 2824, 2825, 2826, 2827, 2828, 2829, 2830, 2831, 2832, 2833, 2834, 2835, 2836, 2837, 2838, 2839, 2840, 2841, 2842, 2843, 2844, 2845, 2846, 2847, 2848, 2849, 2850, 2851, 2852, 2853, 2854, 2855, 2856, 2857, 2858, 2859, 2860, 2861, 2862, 2863, 2864, 2865, 2866, 2867, 2868, 2869, 2870, 2871, 2872, 2873, 2874, 2875, 2876, 2877, 2878, 2879, 2880, 2881, 2882, 2883, 2884, 2885, 2886, 2887, 2888, 2889, 2890, 2891, 2892, 2893, 2894, 2895, 2896, 2897, 2898, 2899, 2900, 2901, 2902, 2903, 2904, 2905, 2906, 2907, 2908, 2909, 2910, 2911, 2912, 2913, 2914, 2915, 2916, 2917, 2918, 2919, 2920, 2921, 2922, 2923, 2924, 2925, 2926, 2927, 2928, 2929, 2930, 2931, 2932, 2933, 2934, 2935, 2936, 2937, 2938, 2939, 2940, 2941, 2942, 2943, 2944, 2945, 2946, 2947, 2948, 2949, 2950, 2951, 2952, 2953, 2954, 2955, 2956, 2957, 2958, 2959, 2960, 2961, 2962, 2963, 2964, 2965, 2966, 2967, 2968, 2969, 2970, 2971, 2972, 2973, 2974, 2975, 2976, 2977, 2978, 2979, 2980, 2981, 2982, 2983, 2984, 2985, 2986, 2987, 2988, 2989, 2990, 2991, 2992, 2993, 2994, 2995, 2996, 2997, 2998, 2999, 3000, 3001, 3002, 3003, 3004, 3005, 3006, 3007, 3008, 3009, 3010, 3011, 3012, 3013, 3014, 3015, 3016, 3017, 3018, 3019, 3020, 3021, 3022, 3023, 3024, 3025, 3026, 3027, 3028, 3029, 3030, 3031, 3032, 3033, 3034, 3035, 3036, 3037, 3038, 3039, 3040, 3041, 3042, 3043, 3044, 3045, 3046, 3047, 3048, 3049, 3050, 3051, 3052, 3053, 3054, 3055, 3056, 3057, 3058, 3059, 3060, 3061, 3062, 3063, 3064, 3065, 3066, 3067, 3068, 3069, 3070, 3071, 3072, 3073, 3074, 3075, 3076, 3077, 3078, 3079, 3080, 3081, 3082, 3083, 3084, 3085, 3086, 3087, 3088, 3089, 3090, 3091, 3092, 3093, 3094, 3095, 3096, 3097, 3098, 3099, 3100, 3101, 3102, 3103, 3104, 3105, 3106, 3107, 3108, 3109, 3110, 3111, 3112, 3113, 3114, 3115, 3116, 3117, 3118, 3119, 3120, 3121, 3122, 3123, 3124, 3125, 3126, 3127, 3128, 3129, 3130, 3131, 3132, 3133, 3134, 3135, 3136, 3137, 3138, 3139, 3140, 3141, 3142, 3143, 3144, 3145, 3146, 3147, 3148, 3149, 3150, 3151, 3152, 3153, 3154, 3155, 3156, 3157, 3158, 3159, 3160, 3161, 3162, 3163, 3164, 3165, 3166, 3167, 3168, 3169, 3170, 3171, 3172, 3173, 3174, 3175, 3176, 3177, 3178, 3179, 3180, 3181, 3182, 3183, 3184, 3185, 3186, 3187, 3188, 3189, 3190, 3191, 3192, 3193, 3194, 3195, 3196, 3197, 3198, 3199, 3200, 3201, 3202, 3203, 3204, 3205, 3206, 3207, 3208, 3209, 3210, 3211, 3212, 3213, 3214, 3215, 3216, 3217, 3218, 3219, 3220, 3221, 3222, 3223, 3224, 3225, 3226, 3227, 3228, 3229, 3230, 3231, 3232, 3233, 3234, 3235, 3236, 3237, 3238, 3239, 3240, 3241, 3242, 3243, 3244, 3245, 3246, 3247, 3248, 3249, and 3250 nucleotides. The length of any filler region for the viral genome may be 50-100, 100-150, 150-200, 200-250, 250-300, 300-350, 350-400, 400-450, 450-500, 500-550, 550-600, 600-650, 650-700, 700-750, 750-800, 800-850, 850-900, 900-950, 950-1000, 1000-1050, 1050-1100, 1100-1150, 1150-1200, 1200-1250, 1250-1300, 1300-1350, 1350-1400, 1400-1450, 1450-1500, 1500-1550, 1550-1600, 1600-1650, 1650-1700, 1700-1750, 1750-1800, 1800-1850, 1850-1900, 1900-1950, 1950-2000, 2000-2050, 2050-2100, 2100-2150, 2150-2200, 2200-2250, 2250-2300, 2300-2350, 2350-2400, 2400-2450, 2450-2500, 2500-2550, 2550-2600, 2600-2650, 2650-2700, 2700-2750, 2750-2800, 2800-2850, 2850-2900, 2900-2950, 2950-3000, 3000-3050, 3050-3100, 3100-3150, 3150-3200, and 3200-3250 nucleotides, As a non-limiting example, the viral genome comprises a filler region that is about 55 nucleotides in length. As a non-limiting example, the viral genome comprises a filler region that is about 56 nucleotides in length. As a non-limiting example, the viral genome comprises a filler region that is about 97 nucleotides in length. As a non-limiting example, the viral genome comprises a filler region that is about 103 nucleotides in length. As a non-limiting example, the viral genome comprises a filler region that is about 105 nucleotides in length. As a non-limiting example, the viral genome comprises a filler region that is about 357 nucleotides in length. As a non-limiting example, the viral genome comprises a filler region that is about 363 nucleotides in length. As a non-limiting example, the viral genome comprises a filler region that is about 712 nucleotides in length. As a non-limiting example, the viral genome comprises a filler region that is about 714 nucleotides in length. As a non-limiting, example, the viral genome comprises a filler region that is about 1203 nucleotides in length. As a non-limiting example, the viral genome comprises a filler region that is about 1209 nucleotides in length. As a non-limiting example, the viral genome comprises a filler region that is about 1512 nucleotides in length. As a non-limiting example, the viral genome comprises a filler region that is about 1519 nucleotides in length. As a non-limiting example, the viral genome comprises a filler region that is about 2395 nucleotides in length. As a non-limiting example, the viral genome comprises a filler region that is about 2403 nucleotides in length. As a non-limiting example, the viral genome comprises a filler region that is about 2405 nucleotides in length. As a non-limiting example, the viral genome comprises a filler region that is about 3013 nucleotides in length. As a non-limiting example, the viral genome comprises a filler region that is about 3021 nucleotides in length.

In some embodiments, the AAV particle viral genome may comprise at least one enhancer sequence region. The enhancer sequence region(s) may, independently, have a length such as, but not limited to, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, and 400 nucleotides. The length of the enhancer region for the viral genome may be 300-310, 300-325, 305-315, 310-320, 315-325, 320-330, 325-335, 325-350, 330-340, 335-345, 340-350, 345-355, 350-360, 350-375, 355-365, 360-370, 365-375, 370-380, 375-385, 375-400, 380-390, 385-395, and 390-400 nucleotides. As a non-limiting example, the viral genome comprises an enhancer region that is about 303 nucleotides in length. As a non-limiting example, the viral genome comprises an enhancer region that is about 382 nucleotides in length.

In some embodiments, the AAV particle viral genome comprises at least one enhancer sequence region. Non-limiting examples of enhancer sequence regions are described in Table 15.

TABLE 15
Enhancer Sequence Regions

| Sequence Region Name | SEQ ID NO |
|---|---|
| Enhancer1 | 1408 |
| Enhancer2 | 1409 |

In some embodiments, the AAV particle viral genome comprises one enhancer sequence region. In some embodiments, the enhancer sequence region is the Enhancer1 sequence region. In some embodiments, the enhancer sequence region is the Enhancer1 sequence region.

In some embodiments, the AAV particle viral genome comprises two enhancer sequence regions. In some embodiments, the enhancer sequence regions are the Enhancer1 sequence region and the Enhancer 2 sequence region.

In some embodiments, the AAV particle viral genome may comprise at least one promoter sequence region. The promoter sequence region(s) may, independently, have a length such as, but not limited to, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, and 600 nucleotides. The length of the promoter region for the viral genome may be 4-10, 10-20, 10-50, 20-30, 30-40, 40-50, 50-60, 50-100, 60-70, 70-80, 80-90, 90-100, 100-110, 100-150, 110-120, 120-130, 130-140, 140-150, 150-160, 150-200, 160-170, 170-180, 180-190, 190-200, 200-210, 200-250, 210-220, 220-230, 230-240, 240-250, 250-260, 250-300, 260-270, 270-280, 280-290, 290-300, 300-310, 300-350, 310-320, 320-330, 330-340, 340-350, 350-360, 350-400, 360-370, 370-380, 380-390, 390-400, 400-410, 400-450, 410-420, 420-430, 430-440, 440-450, 450-460, 450-500, 460-470, 470-480, 480-490, 490-500, 500-510, 500-550, 510-520, 520-530, 530-540, 540-550, 550-560, 550-600, 560-570, 570-580, 580-590, and 590-600 nucleotides. As a non-limiting example, the viral genome comprises a promoter region that is about 4 nucleotides in length. As a non-limiting example, the viral genome comprises a promoter region that is about 17 nucleotides in length. As a non-limiting example, the viral genome comprises a promoter region that is about 204 nucleotides in length. As a non-limiting example, the viral genome comprises a promoter region that is about 219 nucleotides in length. As a non-limiting example, the viral genome comprises a promoter region that is about 260 nucleotides in length. As a non-limiting example, the viral genome comprises a promoter region that is about 303 nucleotides in length. As a non-limiting example, the viral genome comprises a promoter region that is about 382 nucleotides in length. As a non-limiting example, the viral genome comprises a promoter region that is about 588 nucleotides in length.

In some embodiments, the AAV particle viral genome comprises at least one promoter sequence region. Non-limiting examples of promoter sequence regions are described in Table 16.

TABLE 16
Promoter Sequence Regions

| | SEQ ID NO or |
|---|---|
| Sequence Region Name | Sequence |
| Promoter1 | 1410 |
| Promoter2 | 1411 |
| Promoter3 | GTTG |
| Promoter4 | 1412 |
| Promoter5 | 1413 |
| Promoter6 | 1414 |

In some embodiments, the AAV particle viral genome comprises one promoter sequence region. In some embodiments, the promoter sequence region is Promoted. In some embodiments, the promoter sequence region is Promoter2. in some embodiments, the promoter sequence region is Promoter3. In some embodiments, the promoter sequence region is Promoter4. In some embodiments, the promoter sequence region is Promoter5. In some embodiments, the promoter sequence region is Promoter6.

In some embodiments, the AAV particle viral genome comprises two promoter sequence regions. In some embodiments, the promoter sequence region is Promoter1 sequence region, and the Promoter2 sequence region. In some embodiments, the promoter sequence region is Promoter1 sequence region, and the Promoter3 sequence region. In some embodiments, the promoter sequence region is Promoter1 sequence region, and the Promoter4 sequence region. In some embodiments, the promoter sequence region is Promoter1 sequence region, and the Promoter5 sequence region. In some embodiments, the promoter sequence region is Promoter1 sequence region, and the Promoter6 sequence region. In some embodiments, the promoter sequence region is Promoter2 sequence region, and the Promoter3 sequence region. In some embodiments, the promoter sequence region is Promoter2 sequence region, and the Promoter4 sequence region. In some embodiments, the promoter sequence region is Promoter2 sequence region, and the Promoter5 sequence region. In some embodiments, the promoter sequence region is Promoter2 sequence region, and the Promoter6 sequence region. In some embodiments, the promoter sequence region is Promoter3 sequence region, and the Promoter4 sequence region. In some embodiments, the promoter sequence region is Promoter3 sequence region, and the Promoter5 sequence region. In some embodiments, the promoter sequence region is Promoter3 sequence region, and the Promoter6 sequence region. In some embodiments, the promoter sequence region is Promoter4 sequence region, and the Promoter5 sequence region. In some embodiments, the promoter sequence region is Promoter4 sequence region, and the Promoter6 sequence region. In some embodiments, the promoter sequence region is Promoter5 sequence region, and the Promoter6 sequence region.

In some embodiments, the AAV particle viral genome may comprise at least one exon sequence region. The exon region(s) may, independently, have a length such as, but not limited to, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, and 150 nucleotides. The length of the exon region for the viral genome may be 2-10, 5-10, 5-15, 10-20, 10-30, 10-40, 15-20, 15-25, 20-30, 20-40, 20-50, 25-30, 25-35, 30-40, 30-50, 30-60, 35-40, 35-45, 40-50, 40-60, 40-70, 45-50, 45-55, 50-60, 50-70, 50-80, 55-60, 55-65, 60-70, 60-80, 60-90, 65-70, 65-75, 70-80, 70-90, 70-100, 75-80, 75-85, 80-90, 80-100, 80-110, 85-90, 85-95, 90-100, 90-110, 90-120, 95-100, 95-105, 100-110, 100-120, 100-130, 105-110, 105-115, 110-120, 110-130, 110-140, 11--120, 115-125, 120-130, 120-140, 120-150, 125-130, 125-135, 130-140, 130-150, 135-140, 135-145, 140-150, and 145-150 nucleotides. As a non-limiting example, the viral genome comprises an exon region that is about 53 nucleotides in length. As a non-limiting example, the viral genome comprises an exon region that is about 134 nucleotides in length.

In some embodiments, the AAV particle viral genome comprises at least one Exon sequence region. Non-limiting examples of Exon sequence regions are described in Table 17.

TABLE 17
Exon Sequence Regions

| Sequence Region Name | SEQ ID NO |
|---|---|
| Exon1 | 1415 |
| Exon2 | 1416 |

In some embodiments, the AAV particle viral genome comprises one Exon sequence region. In some embodiments, the Exon sequence regions is the Exon1 sequence region. In some embodiments, the Exon sequence regions is the Exon2 sequence region,

In some embodiments, the AAV particle viral genome comprises two Exon sequence regions. In some embodiments, the Exon sequence regions are the Exon1 sequence region and the Exon 2 sequence region.

In some embodiments, the AAV particle viral genome may comprise at least one intron sequence region. The intron region(s) may, independently, have a length such as, but not limited to, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, and 350 nucleotides. The length of the intron region for the viral genome may be 25-35, 25-50, 35-45, 45-55, 50-75, 55-65, 65-75, 75-85, 75-100, 85-95, 95-105, 100-125, 105-115, 115-125, 125-135, 125-150, 135-145, 145-155, 150-175, 155-165, 165-175, 175-185, 175-200, 185-195, 195-205, 200-225, 205-215, 215-225, 225-235, 225-250, 235-245, 245-255, 250-275, 255-265, 265-275, 275-285, 275-300, 285-295, 295-305, 300-325, 305-315, 315-325, 325-335, 325-350, and 335-345 nucleotides. As a non-limiting example, the viral genome comprises an intron region that is about 32 nucleotides in length. As a non-limiting example, the viral genome comprises an intron region that is about 172 nucleotides in length. As a non-limiting example, the viral genome comprises an intron region that is about 201 nucleotides in length. As a non-limiting example, the viral genome comprises an intron region that is about 347 nucleotides in length.

In some embodiments, the AAV particle viral genome comprises at least one intron sequence region. Non-limiting examples of intron sequence regions are described in Table 18.

TABLE 18
Intron Sequence Regions

| Sequence Region Name | SEQ ID NO |
|---|---|
| Intron1 | 1417 |
| Intron2 | 1418 |
| Intron3 | 1419 |

In some embodiments, the AAV particle viral genome comprises one intron sequence region. In some embodiments, the intron sequence region is the Intron1 sequence region. In some embodiments, the intron sequence region is the Intron2 sequence region. In some embodiments, the intron sequence region is the Intron3 sequence region.

In some embodiments, the AAV particle viral genome comprises two intron sequence regions. In some embodiments, the intron sequence regions are the Intron1 sequence region and the Intron2 sequence region. In some embodiments, the intron sequence regions are the Intron2 sequence region and the Intron3 sequence region. In some embodiments, the intron sequence regions are the Intron1 sequence region and the Intron3 sequence region.

In some embodiments, the AAV particle viral genome comprises three intron sequence regions. In some embodiments, the intron sequence regions are the Intron1 sequence region, the Intron2 sequence region, and the Intron3 sequence region.

In some embodiments, the AAV particle viral genome may comprise at least one polyadenylation signal sequence region. The polyadenylation signal region sequence region(s) may, independently, have a length such as, but not limited to, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 323, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, and 600 nucleotides. The length of the polyadenylation signal sequence region for the viral genome may be 4-10, 10-20, 10-50, 20-30, 30-40, 40-50, 50-60, 50-100, 60-70, 70-80, 80-90, 90-100, 100-110, 100-150, 110-120, 120-130, 130-140, 140-150, 150-160, 150-200, 160-170, 170-180, 180-190, 190-200, 200-210, 200-250, 210-220, 220-230, 230-240, 240-250, 250-260, 250-300, 260-270, 270-280, 280-290, 290-300, 300-310, 300-350, 310-320, 320-330, 330-340, 340-350, 350-360, 350-400, 360-370, 370-380, 380-390, 390-400, 400-410, 400-450, 410-420, 420-430, 430-440, 440-450, 450-460, 450-500, 460-470, 470-480, 480-490, 490-500, 500-510, 500-550, 510-520, 520-530, 530-540, 540-550, 550-560, 550-600, 560-570, 570-580, 580-590, and 590-600 nucleotides. As a non-limiting example, the viral genome comprises a polyadenylation signal sequence region that is about 127 nucleotides in length. As a non-limiting, example, the viral genome comprises a polyadenylation signal sequence region that is about 225 nucleotides in length. As a non-limiting example, the viral genome comprises a polyadenylation signal sequence region that is about 476 nucleotides in length. As a non-limiting example, the viral genome comprises a polyadenylation signal sequence region that is about 477 nucleotides in length.

In some embodiments, the AAV particle viral genome comprises at least one polyadenylation (polyA) signal sequence region. Non-limiting examples of polyA signal sequence regions are described in Table 19.

TABLE 19
PolyA Signal Sequence Regions

| Sequence Region Name | SEQ ID NO |
|---|---|
| PolyA1 | 1420 |
| PolyA2 | 1421 |
| PolyA3 | 1422 |
| PolyA4 | 1423 |

In some embodiments, the AAV particle viral genome comprises one polyA signal sequence region. In some embodiments, the polyA signal sequence regions is the PolyA.1 sequence region. In some embodiments, the polyA signal sequence regions is the PolyA2 sequence region. In some embodiments, the polyA signal sequence regions is the PolyA3 sequence region. In some embodiments, the polyA signal sequence regions is the PolyA4 sequence region.

In some embodiments, the AAV particle viral genome comprises more than one polyA signal sequence region.

In some embodiments, the AAV particle viral genome comprises at least one inverted terminal repeat (ITR) sequence region, at least one multiple cloning site (MCS) sequence region, at least one enhancer sequence region, at least one promoter sequence region, at least one exon sequence region, at least one intron sequence region, at least one modulatory polynucleotide region, at least one polyadenylation signal sequence region, and at least one filler sequence region,

In some embodiments, the AAV particle viral genome comprises a 5′ inverted terminal repeat (ITR) sequence region and a 3′ ITR sequence region, two multiple cloning site (MCS) sequence regions, an enhancer sequence region, a promoter sequence region, an intron sequence region, a modulatory polynucleotide region, a polyadenylation signal sequence region, and a filler sequence region.

In some embodiments, the AAV particle viral genome comprises a 5′ inverted terminal repeat (ITR) sequence region and a 3′ ITR sequence region, two multiple cloning site (MCS) sequence regions, a CMV enhancer sequence region, a CBA promoter sequence region, an SV40 intron sequence region, a modulatory polynucleotide region, a rabbit globin polyadenylation signal sequence region, and a filler sequence region. Non-limiting examples of ITR to ITR sequences for use in the AAV particles of the present disclosure having all of the sequence modules above are described in Table 20. In Table 20, the sequence identifier or sequence of the sequence region (Region SEQ ID NO) and the length of the sequence region (Region length) are described as well as the name and sequence identifier of the ITR to ITR sequence (e.g., VOYHT1 (SEQ ID NO: 1352)).

TABLE 20
Sequence Regions in ITR to ITR Sequences

| | VOYHT1 (SEQ | | VOYHT2 (SEQ | | VOYHT3 (SEQ | | VOYHT4 (SEQ | |
|---|---|---|---|---|---|---|---|---|
| | ID NO: 1352) | | ID NO: 1353) | | ID NO: 1354) | | ID NO: 1355) | |
| Sequence | Region SEQ | Region | Region SEQ | Region | Region SEQ | Region | Region SEQ | Region |
| Regions | ID NO | length | ID NO | length | ID NO | length | ID NO | length |
| 5′ ITR | 1380 | 141 | 1380 | 141 | 1380 | 141 | 1380 | 141 |
| MCS | 1384 | 10 | 1384 | 10 | 1384 | 10 | 1384 | 10 |
| CMV enhancer | 1408 | 382 | 1408 | 382 | 1408 | 382 | 1408 | 382 |
| CBA Promoter | 1410 | 260 | 1410 | 260 | 1410 | 260 | 1410 | 260 |
| SV40 Intron | 1417 | 172 | 1417 | 172 | 1417 | 172 | 1417 | 172 |
| Modulatory | 1262 | 163 | 1262 | 163 | 1250 | 158 | 1347 | 163 |
| Polynucleotide | | | | | | | | |
| MCS | TCGAG | 5 | TCGAG | 5 | TCGAG | 5 | TCGAG | 5 |
| Rabbit globin | 1420 | 127 | 1420 | 127 | 1420 | 127 | 1420 | 127 |
| PolyA Signal | | | | | | | | |
| 3′ ITR | 1382 | 141 | 1382 | 141 | 1382 | 141 | 1382 | 141 |

In some embodiments, the AAV particle viral genome comprises SEQ ID NO: 1352 (VOYHT1) which comprises a 5′ inverted terminal repeat (ITR) sequence region and a 3′ ITR sequence region, two multiple cloning site (MCS) sequence regions, a CMV enhancer sequence region, a CBA promoter sequence region, an SV40 intron sequence region, a modulatory polynucleotide region, a rabbit globin polyadenylation signal sequence region, and a filler sequence region.

In some embodiments, VOYHT1 is referred to as VY-HTT01. In some embodiments, VY-HTT01 has a CAS (Chemical Abstracts Service) Registry Number of 2288462-09-2.

In some embodiments, the AAV particle viral genome comprises SEQ ID NO: 1353 (VOYHT2) which comprises a 5′ inverted terminal repeat (ITR) sequence region and a 3′ ITR sequence region, two multiple cloning site (MCS) sequence regions, a CMV enhancer sequence region, a CBA promoter sequence region, an SV40 intron sequence region, a modulatory polynucleotide region, a rabbit globin polyadenylation signal sequence region, and a filler sequence region.

In some embodiments, the AAV particle viral genome comprises SEQ ID NO: 1354 (VOYHT3) which comprises a 5′ inverted terminal repeat (ITR) sequence region and a 3′ ITR sequence region, two multiple cloning site (MCS) sequence regions, a CMV enhancer sequence region, a CBA promoter sequence region, an SV40 intron sequence region, a modulatory polynucleotide region, a rabbit globin polyadenylation signal sequence region, and a filler sequence region.

In some embodiments, the AAV particle viral genome comprises SEQ ID NO: 1355 (VOYHT4) which comprises a 5′ inverted terminal repeat (ITR) sequence region and a 3′ ITR sequence region, two multiple cloning site (MCS) sequence regions, a CMV enhancer sequence region, a CBA promoter sequence region, an SV40 intron sequence region, a modulatory polynucleotide region, a rabbit globin polyadenylation signal sequence region, and a filler sequence region.

In some embodiments, the AAV particle viral genome comprises a 5′ inverted terminal repeat (ITR) sequence region and a 3′ ITR sequence region, two multiple cloning site (MCS) sequence regions, a CMV enhancer sequence region, a CBA promoter sequence region, an SV40 intron sequence region, a modulatory polynucleotide region, a rabbit globin polyadenylation signal sequence region, and a filler sequence region. Non-limiting examples of ITR to ITR sequences for use in the AAV particles of the present disclosure having all of the sequence modules above are described in Table 21. In Table 21, the sequence identifier or sequence of the sequence region (Region SEQ m NO) and the length of the sequence region (Region length) are described as well as the name and sequence identifier of the ITR to ITR sequence (e.g., VOYHT5 (SEQ ID NO: 1356)).

TABLE 21
Sequence Regions in ITR to ITR Sequences

| | VOYHT5 (SEQ | | VOYHT6 (SEQ | | VOYHT7 (SEQ | | VOYHT8 (SEQ | |
|---|---|---|---|---|---|---|---|---|
| | ID NO: 1356) | | ID NO: 1357) | | ID NO: 1358) | | ID NO: 1359) | |
| Sequence | Region SEQ | Region | Region SEQ | Region | Region SEQ | Region | Region SEQ | Region |
| Regions | ID NO | length | ID NO | length | ID NO | length | ID NO | length |
| 5′ ITR | 1380 | 141 | 1380 | 141 | 1380 | 141 | 1380 | 141 |
| MCS | 1384 | 10 | 1384 | 10 | 1384 | 10 | 1384 | 10 |
| CMV enhancer | 1408 | 382 | 1408 | 382 | 1408 | 382 | 1408 | 382 |
| CBA Promoter | 1410 | 260 | 1410 | 260 | 1410 | 260 | 1410 | 260 |
| SV40 Intron | 1417 | 172 | 1417 | 172 | 1417 | 172 | 1417 | 172 |
| Modulatory | 1262 | 163 | 1262 | 163 | 1250 | 158 | 1347 | 163 |
| Polynucleotide | | | | | | | | |
| MCS | TCGAG | 5 | TCGAG | 5 | TCGAG | 5 | TCGAG | 5 |
| Rabbit globin | 1420 | 127 | 1420 | 127 | 1420 | 127 | 1420 | 127 |
| PolyA Signal | | | | | | | | |
| 3′ ITR | 1382 | 141 | 1382 | 141 | 1382 | 141 | 1382 | 141 |

In some embodiments, the AAV particle viral genome comprises SEQ m NO: 1356 (VOYHT5) which comprises a 5′ inverted terminal repeat (ITR) sequence region and a 3′ ITR sequence region, two multiple cloning site (MCS) sequence regions, a CMV enhancer sequence region, a CBA promoter sequence region, an SV40 intron sequence region, a modulatory polynucleotide region, a rabbit globin polyadenylation signal sequence region, and a filler sequence region.

In some embodiments, the AAV particle viral genome comprises SEQ ID NO: 1357 (VOYHT6) which comprises a 5′ inverted terminal repeat (ITR) sequence region and a 3′ ITR sequence region, two multiple cloning site (MCS) sequence regions, a CMV enhancer sequence region, a CBA promoter sequence region, an SV40 intron sequence region, a modulatory polynucleotide region, a rabbit globin polyadenylation signal sequence region, and a filler sequence region.

In some embodiments, the AAV particle viral genome comprises SEQ ID NO: 1358 (VOYHT7) which comprises a 5′ inverted terminal repeat (ITR) sequence region and a 3′ ITR sequence region, two multiple cloning site (MCS) sequence regions, a CMV enhancer sequence region, a CBA promoter sequence region, an SV40 intron sequence region, a modulatory polynucleotide region, a rabbit globin polyadenylation signal sequence region, and a filler sequence region.

In some embodiments, the AAV particle viral genome comprises SEQ ID NO: 1359 (VOYHT8) which comprises a 5′ inverted terminal repeat (ITR) sequence region and a 3′ ITR sequence region, two multiple cloning site (MCS) sequence regions, a CMV enhancer sequence region, a CBA promoter sequence region, an SV40 intron sequence region, a modulatory polynucleotide region, a rabbit globin polyadenylation signal sequence region, and a filler sequence region.

In some embodiments, the AAV particle viral genome comprises a 5′ inverted terminal repeat (ITR) sequence region and a 3′ ITR sequence region, two multiple cloning site (MCS) sequence regions, an enhancer sequence region, a promoter sequence region, an intron sequence region, a modulatory polynucleotide region, a polyadenylation signal sequence region, and two filler sequence regions.

In some embodiments, the AAV particle viral genome comprises a 5′ inverted terminal repeat (ITR) sequence region and a 3′ ITR sequence region, two multiple cloning site (MCS) sequence regions, a CMV enhancer sequence region, a CBA promoter sequence region, an SV40 intron sequence region, a modulatory polynucleotide region, a rabbit globin polyadenylation signal sequence region, and two filler sequence regions. Non-limiting examples of ITR to ITR sequences for use in the AAV particles of the present disclosure having all of the sequence modules above are described in Table 22. In Table 22, the sequence identifier or sequence of the sequence region (Region SEQ ID NO) and the length of the sequence region (Region length) are described as well as the name and sequence identifier of the ITR to ITR sequence (e.g., VOYHT9 (SEQ ID NO: 1360)).

TABLE 22
Sequence Regions in ITR to ITR Sequences

| | VOYHT9 (SEQ | | VOYHT10 (SEQ | | VOYHT11 (SEQ | | VOYHT12 (SEQ | |
|---|---|---|---|---|---|---|---|---|
| | ID NO: 1360) | | ID NO: 1361) | | ID NO: 1362) | | ID NO: 1363) | |
| Sequence | Region SEQ | Region | Region SEQ | Region | Region SEQ | Region | Region SEQ | Region |
| Regions | ID NO | length | ID NO | length | ID NO | length | ID NO | length |
| 5′ ITR | 1380 | 141 | 1380 | 141 | 1380 | 141 | 1380 | 141 |
| MCS | 1384 | 10 | 1384 | 10 | 1384 | 10 | 1384 | 10 |
| CMV enhancer | 1408 | 382 | 1408 | 382 | 1408 | 382 | 1408 | 382 |
| CBA Promoter | 1410 | 260 | 1410 | 260 | 1410 | 260 | 1410 | 260 |
| SV40 Intron | 1417 | 172 | 1417 | 172 | 1417 | 172 | 1417 | 172 |
| Modulatory | 1262 | 163 | 1262 | 163 | 1250 | 158 | 1347 | 163 |
| Polynucleotide | | | | | | | | |
| MCS | TCGAG | 5 | TCGAG | 5 | TCGAG | 5 | TCGAG | 5 |
| Rabbit globin | 1420 | 127 | 1420 | 127 | 1420 | 127 | 1420 | 127 |
| PolyA Signal | | | | | | | | |
| 3′ ITR | 1382 | 141 | 1382 | 141 | 1382 | 141 | 1382 | 141 |

In some embodiments, the AAV particle viral genome comprises SEQ ID NO: 1360 (VOYHT9) which comprises a 5′ inverted terminal repeat (ITR) sequence region and a 3′ ITR sequence region, two multiple cloning site (MCS) sequence regions, a CMV enhancer sequence region, a CBA promoter sequence region, an SV40 intron sequence region, a modulatory polynucleotide region, a rabbit globin polyadenylation signal sequence region, and two filler sequence regions.

In some embodiments, the AAV particle viral genome comprises SEQ ID NO: 1361 (VOYHT10) which comprises a 5′ inverted terminal repeat (ITR) sequence region and a 3′ ITR sequence region, two multiple cloning site (MCS) sequence regions, a CMV enhancer sequence region, a CBA promoter sequence region, an SV40 intron sequence region, a modulatory polynucleotide region, a rabbit globin polyadenylation signal sequence region, and two filler sequence regions.

In some embodiments, the AAV particle viral genome comprises SEQ ID NO: 1362 (VOYHT11 ) which comprises a 5′ inverted terminal repeat (ITR) sequence region and a 3′ ITR sequence region, two multiple cloning site (MCS) sequence regions, a CMV enhancer sequence region, a CBA promoter sequence region, an SV40 intron sequence region, a modulatory polynucleotide region, a rabbit globin polyadenylation signal sequence region, and two filler sequence regions.

In some embodiments, the AAV particle viral genome, comprises SEQ ID NO: 1363 (VOYHT12) which comprises a 5′ inverted terminal repeat (ITR) sequence region and a 3′ ITR sequence region, two multiple cloning site (MCS) sequence regions, a CMV enhancer sequence region, a CBA promoter sequence region, an SV40 intron sequence region, a modulatory polynucleotide region, a rabbit globin polyadenylation signal sequence region, and two filler sequence regions.

In some embodiments, the AAV particle viral genome comprises a 5′ inverted terminal repeat (ITR) sequence region and a 3′ ITR sequence region, two multiple cloning site (MCS) sequence regions, an enhancer sequence region, a promoter sequence region, an intron sequence region, a modulatory polynucleotide region, and a polyadenylation signal sequence region.

In some embodiments, the AAV particle viral genome comprises a 5′ inverted terminal repeat (ITR) sequence region and a 3′ ITR sequence region, two multiple cloning site (MCS) sequence regions, a CMV enhancer sequence region, a CBA promoter sequence region, an SV40 intron sequence region, a modulatory polynucleotide region, and a rabbit globin polyadenylation signal sequence region. Non-limiting examples of ITR to ITR sequences for use in the AAV particles of the present disclosure having all of the sequence modules above are described in Table 23. In Table 23, the sequence identifier or sequence of the sequence region (Region SEQ ID NO) and the length of the sequence region (Region length) are described as well as the name and sequence identifier of the ITR to ITR sequence (e.g., VOYHT13 (SEQ NO: 1364)).

TABLE 23
Sequence Regions in ITR to ITR Sequences

| | VOYHT13 (SEQ | | VOYHT14 (SEQ | |
|---|---|---|---|---|
| | ID NO: 1364) | | ID NO: 1365) | |
| Sequence | Region SEQ | Region | Region SEQ | Region |
| Regions | ID NO | length | ID NO | length |
| 5′ ITR | 1380 | 141 | 1380 | 141 |
| MCS | 1384 | 10 | 1384 | 10 |
| CMV | 1408 | 382 | 1408 | 382 |
| enhancer | | | | |
| CBA Promoter | 1410 | 260 | 1410 | 260 |
| SV40 Intron | 1417 | 172 | 1417 | 172 |
| Modulatory | 1262 | 163 | 1249 | 158 |
| Polynucleotide | | | | |
| MCS | TCGAG | 5 | TCGAG | 5 |
| Rabbit globin | 1420 | 127 | 1420 | 127 |
| PolyA Signal | | | | |
| 3′ ITR | 1382 | 141 | 1382 | 141 |

In some embodiments, the AAV particle viral genome comprises SEQ ID NO: 1364 (VOYHT13) which comprises a 5′ inverted terminal repeat (ITR)) sequence region and a 3′ ITR sequence region, two multiple cloning site (MCS) sequence regions, a CMV enhancer sequence region, a CBA promoter sequence region, an SV40 intron sequence region, a modulatory polynucleotide region, and a rabbit globin polyadenylation signal sequence region.

In some embodiments, the AAV particle viral genome comprises SEQ ID NO: 1365 (VOYHT14) which comprises a 5′ inverted terminal repeat (ITR) sequence region and a 3′ ITR sequence region, two multiple cloning site (MCS) sequence regions, a CMV enhancer sequence region, a CBA promoter sequence region, an SV40 intron sequence region, a modulatory polynucleotide region, and a rabbit globin polyadenylation signal sequence region.

In some embodiments, the AAV particle viral genome comprises a 5′ inverted terminal repeat (ITR) sequence region and a 3′ ITR sequence region, an enhancer sequence region, a promoter sequence region, an intron sequence region, a modulatory polynucleotide region, and a polyadenylation signal sequence region.

In some embodiments, the AAV particle viral genome comprises a 5′ inverted terminal repeat (ITR) sequence region and a 3′ ITR sequence region, a CMV enhancer sequence region, a CBA promoter sequence region, an SV40 intron sequence region, a modulatory polynucleotide region, and a rabbit globin polyadenylation signal sequence region. Non-limiting examples of ITR to ITR sequences for use in the AAV particles of the present disclosure having all of the sequence modules above are described in Tables 24-28. In Tables 24-28, the sequence identifier or sequence of the sequence region (Region SEQ ID NO) and the length of the sequence region (Region length) are described as well as the name and sequence identifier of the ITR to ITR sequence e.g., VOYHT15 (SEQ ID NO: 1366)).

TABLE 24
Sequence Regions in ITR to ITR Sequences

| | VOYHT15 (SEQ | | VOYHT16 (SEQ | |
|---|---|---|---|---|
| | ID NO: 1366) | | ID NO: 1367) | |
| Sequence | Region SEQ | Region | Region SEQ | Region |
| Regions | ID NO | length | ID NO | length |
| 5′ ITR | 1381 | 105 | 1381 | 105 |
| CMV enhancer | 1408 | 382 | 1408 | 382 |
| CBA Promoter | 1410 | 260 | 1410 | 260 |
| SV40 intron | 1417 | 172 | 1417 | 172 |
| Modulatory | 1259 | 260 | 1249 | 158 |
| Polynucleotide | | | | |
| Rabbit globin | 1420 | 127 | 1420 | 127 |
| PolyA Signal | | | | |
| 3′ ITR | 1383 | 130 | 1383 | 130 |

TABLE 25
Sequence Regions in ITR to ITR Sequences

| | VOYHT35 (SEQ | | VOYHT36 (SEQ | | VOYHT37 (SEQ | | VOYHT38 (SEQ | |
|---|---|---|---|---|---|---|---|---|
| | ID NO: 1388) | | ID NO. 1426) | | ID NO: 1427) | | ID NO: 1428) | |
| Sequence | RegionSEQ | Region | Region SEQ | Region | Region SEQ | Region | Region SEQ | Region |
| Regions | ID NO | length | ID NO | length | ID NO | length | ID NO | length |
| 5′ ITR | 1381 | 105 | 1381 | 105 | 1381 | 105 | 1381 | 105 |
| CMV enhancer | 1408 | 382 | 1408 | 382 | 1408 | 382 | 1408 | 382 |
| CBA Promoter | 1410 | 260 | 1410 | 260 | 1410 | 260 | 1410 | 260 |
| SV40 Intron | 1417 | 172 | 1417 | 172 | 1417 | 172 | 1417 | 172 |
| Modulatory | 1255 | 158 | 1248 | 158 | 1231 | 158 | 1219 | 158 |
| Polynucleotide | | | | | | | | |
| Rabbit globin | 1420 | 127 | 1420 | 127 | 1420 | 127 | 1420 | 127 |
| PolyA Signal | | | | | | | | |
| 3′ ITR | 1383 | 130 | 1383 | 130 | 1383 | 130 | 1383 | 130 |

TABLE 26
Sequence Regions in ITR to ITR Sequences

| | VOYHT39 (SEQ | | VOYHT40 (SEQ | | VOYHT41 (SEQ | | VOYHT42 (SEQ | |
|---|---|---|---|---|---|---|---|---|
| | ID NO: 1429) | | ID NO: 1430) | | ID NO: 1431) | | ID NO: 1432) | |
| Sequence | Region SEQ | Region | Region SEQ | Region | Region SEQ | Region | Region SEQ | Region |
| Regions | ID NO | length | ID NO | length | ID NO | length | ID NO | length |
| 5′ ITR | 1381 | 105 | 1381 | 105 | 1381 | 105 | 1381 | 105 |
| CMV enhancer | 1408 | 382 | 1408 | 382 | 1408 | 382 | 1408 | 382 |
| CBA Promoter | 1410 | 260 | 1410 | 260 | 1410 | 260 | 1410 | 260 |
| SV40 Intron | 1417 | 172 | 1417 | 172 | 1417 | 172 | 1417 | 172 |
| Modulatory | 1207 | 158 | 1250 | 158 | 1251 | 158 | 1252 | 158 |
| Polynucleotide | | | | | | | | |
| Rabbit globin | 1420 | 127 | 1420 | 127 | 1420 | 127 | 1420 | 127 |
| PolyA Signal | | | | | | | | |
| 3′ ITR | 1383 | 130 | 1383 | 130 | 1383 | 130 | 1383 | 130 |

TABLE 27
Sequence Regions in ITR to ITR Sequences

| | VOYHT43 (SEQ | | VOYHT44 (SEQ | | VOYHT45 (SEQ | | VOYHT46 (SEQ | |
|---|---|---|---|---|---|---|---|---|
| | ID NO: 1433) | | ID NO: 1434) | | ID NO: 1435) | | ID NO: 1436) | |
| Sequence | Region SEQ | Region | Region SEQ | Region | Region SEQ | Region | Region SEQ | Region |
| Regions | ID NO | length | ID NO | length | ID NO | length | ID NO | length |
| 5′ ITR | 1381 | 105 | 1381 | 105 | 1381 | 105 | 1381 | 105 |
| CMV enhancer | 1408 | 382 | 1408 | 382 | 1408 | 382 | 1408 | 382 |
| CBA Promoter | 1410 | 260 | 1410 | 260 | 1410 | 260 | 1410 | 260 |
| SV40 Intron | 1417 | 172 | 1417 | 172 | 1417 | 172 | 1417 | 172 |
| Modulatory | 1253 | 260 | 1194 | 260 | 1223 | 260 | 1211 | 260 |
| Polynucleotide | | | | | | | | |
| Rabbit globin | 1420 | 127 | 1420 | 127 | 1420 | 127 | 1420 | 127 |
| PolyA Signal | | | | | | | | |
| 3′ ITR | 1383 | 130 | 1383 | 130 | 1383 | 130 | 1383 | 130 |

TABLE 28
Sequence Regions in ITR to ITR Sequences

| | VOYHT47 (SEQ | | VOYHT48 (SEQ | |
|---|---|---|---|---|
| | ID NO: 1437) | | ID NO: 1438) | |
| Sequence | Region SEQ | Region | Region SEQ | Region |
| Regions | ID NO | length | ID NO | length |
| 5′ ITR | 1381 | 105 | 1381 | 105 |
| CMV enhancer | 1408 | 382 | 1408 | 382 |
| CBA Promoter | 1410 | 260 | 1410 | 260 |
| SV40 Intron | 1417 | 172 | 1417 | 172 |
| Modulatory | 1262 | 163 | 1347 | 163 |
| Polynucleotide | | | | |
| Rabbit globin | 1420 | 127 | 1420 | 127 |
| PolyA Signal | | | | |
| 3′ ITR | 1383 | 130 | 1383 | 130 |

In some embodiments, the AAV particle viral genome comprises SEQ ID NO: 1366 (VOYHT15) which comprises a 5′ inverted terminal repeat (ITR) sequence region and a 3′ ITR sequence region, a CMV enhancer sequence region, a CBA promoter sequence region, an SV40 intron sequence region, a modulatory polynucleotide region, and a rabbit globin polyadenylation signal sequence region.

In some embodiments, the AAV particle viral genome comprises SEQ ID NO: 1367 (VOYHT16) which comprises a 5′ inverted terminal repeat (ITR) sequence region and a 3′ ITR sequence region, a CMV enhancer sequence region, a CBA promoter sequence region, an 5V40 intron sequence region, a modulatory polynucleotide region, and a rabbit globin polyadenylation signal sequence region.

In some embodiments, the AAV particle viral genome comprises SEQ ID NO: 1388 (VOYHT35) which comprises a 5′ inverted terminal repeat (ITR) sequence region and a 3′ ITR sequence region, a CMV enhancer sequence region, a CBA promoter sequence region, an SV40 intron sequence region, a modulatory polynucleotide region, and a rabbit globin polyadenylation signal sequence region.

in some embodiments, the AAV particle viral genome comprises SEQ ID NO: 1426 (VOYHT36) which comprises a 5′ inverted terminal repeat (ITR)) sequence region and a 3′ ITR sequence region, a CMV enhancer sequence region, a CBA promoter sequence region, an SV40 intron sequence region, a modulatory polynucleotide region, and a rabbit globin polyadenylation signal sequence region.

In some embodiments, the AAV particle viral genome comprises SEQ ID NO: 1427 (VOYHT37) which comprises a 5′ inverted terminal repeat (ITR) sequence region and a 3′ ITR sequence region, a CMV enhancer sequence region, a CBA promoter sequence region, an SV40 intron sequence region, a modulatory polynucleotide, region, and a rabbit globin polyadenylation signal sequence region.

In some embodiments, the AAV particle viral genome comprises SEQ ID NO: 1428 (VOYHT38) which comprises a 5′ inverted terminal repeat (ITR) sequence region and a 3′ ITR sequence region, a CMV enhancer sequence region, a CBA promoter sequence region, an SV40 intron sequence region, a modulatory polynucleotide region, and a rabbit globin polyadenylation signal sequence region.

In some embodiments, the AAV particle viral genome comprises SEQ ID NO: 1429 (VOYHT39) which comprises a 5′ inverted terminal repeat (ITR) sequence region and a 3′ ITR sequence region, a CMV enhancer sequence region, a CBA promoter sequence region, an SV40 intron sequence region, a modulatory polynucleotide region, and a rabbit globin polyadenylation signal sequence region.

In some embodiments, the AAV particle viral genome comprises SEQ m NO: 1430 (VOYHT40) which comprises a 5′ inverted terminal repeat (ITR) sequence region and a 3′ ITR sequence region, a CMV enhancer sequence region, a CBA promoter sequence region, an SV40 intron sequence region, a modulatory polynucleotide, region, and a rabbit globin polyadenylation signal sequence region.

In some embodiments, the AAV particle viral genome comprises SEQ ID NO: 1431 (VOYHT41) which comprises a 5′ inverted terminal repeat (ITR) sequence region and a 3′ ITR sequence region, a CMV enhancer sequence region, a CBA promoter sequence region, an SV40 intron sequence region, a modulatory polynucleotide region, and a rabbit globin polyadenylation signal sequence region.

In some embodiments, the AAV particle viral genome comprises SEQ ID NO: 1432 (VOYHT42) which comprises a 5′ inverted terminal repeat (ITR) sequence region and a 3′ ITR sequence region, a CMV enhancer sequence region, a CBA promoter sequence region, an SV40 intron sequence region, a modulatory polynucleotide region, and a rabbit globin polyadenylation signal sequence region.

In some embodiments, the AAV particle viral genome comprises SEQ ID NO: 1433 (VOYHT43) which comprises a 5′ inverted terminal repeat (ITR) sequence region and a 3′ ITR sequence region, a CMV enhancer sequence region, a CBA promoter sequence region, an SV40 intron sequence region, a modulatory polynucleotide region, and a rabbit globin polyadenylation signal sequence region.

In some embodiments, the AAV particle viral genome comprises SEQ ID NO: 1434 (VOYHT44) which comprises a 5′ inverted terminal repeat (ITR) sequence region and a 3′ ITR sequence region, a CMV enhancer sequence region, a CBA promoter sequence region, an SV40 intron sequence region, a modulatory polynucleotide region, and a rabbit globin polyadenylation signal sequence region.

In some embodiments, the AAV particle viral genome comprises SEQ ID NO: 1435 (VOYHT45) which comprises a 5′ inverted terminal repeat (ITR) sequence region and a 3′ ITR sequence region, a CMV enhancer sequence region, a CBA promoter sequence region, an SV40 intron sequence region, a modulatory polynucleotide region, and a rabbit globin polyadenylation signal sequence region.

In some embodiments, the AAV particle viral genome comprises SEQ ID NO: 1436 (VOYHT46) which comprises a 5′ inverted terminal repeat (ITR) sequence region and a 3′ ITR sequence region, a CMV enhancer sequence region, a CBA promoter sequence region, an SV40 intron sequence region, a modulatory polynucleotide region, and a rabbit globin polyadenylation signal sequence region.

In some embodiments, the AAV particle viral genome comprises SEQ ID NO: 1437 (VOYHT47) which comprises a 5′ inverted terminal repeat (ITR) sequence region and a 3′ ITR sequence region, a CMV enhancer sequence region, a CBA promoter sequence region, an SV40 intron sequence region, a modulatory polynucleotide region, and a rabbit globin polyadenylation signal sequence region.

In some embodiments, the AAV particle viral genome comprises SEQ ID NO: 1438 (VOYHT48) which comprises a 5′ inverted terminal repeat (ITR) sequence region and a 3′ ITR sequence region, a CMV enhancer sequence region, a CBA promoter sequence region, an SV40 intron sequence region, a modulatory polynucleotide region, and a rabbit globin polyadenylation signal sequence region.

In some embodiments, the AAV particle viral genome comprises a 5′ inverted terminal repeat (ITR) sequence region and a 3′ ITR sequence region, a MCS sequence region, an enhancer sequence region, a promoter sequence region, two exon sequence regions, two intron sequence regions, a modulatory polynucleotide region, a polyadenylation signal sequence region, and a filler sequence region.

In some embodiments, the AAV particle viral genome comprises a 5′ inverted terminal repeat (ITR) sequence region and a 3′ ITR sequence region, a MCS sequence region, a CMV enhancer sequence region, a CMV promoter sequence region, two exon sequence regions (ie1 exon 1 and human beta globin (hbglobin) exon 3 or fragments thereof), intron sequence regions (ie1 intron 1 and hbglobin intron 2 or fragments thereof), a modulatory polynucleotide region, a human growth hormone (hGH) polyadenylation signal sequence region, and a filler sequence region. A non-limiting example of an ITR to ITR sequence for use in the AAV particles of the present disclosure having all of the sequence modules above are described in Table 29. In Table 29, the sequence identifier or sequence of the sequence region (Region SEQ ID NO) and the length of the sequence region (Region length) arc described as well as the name and sequence identifier of the ITR to ITR sequence g., VOYHT17 (SEQ ID NO: 1368)).

TABLE 29
Sequence Regions in ITR to ITR Sequences

VOYHT17 (SEQ
ID NO: 1368)

| Sequence | Region SEQ | Region |
|---|---|---|
| Regions | ID NO | length |
| 5′ ITR | 1380 | 141 |
| MCS | 1387 | 18 |
| CMV enhancer | 1409 | 303 |
| CMV Promoter | 1412 | 204 |
| Ie1 exon1 | 1415 | 134 |
| Ie1 intron1 | 1418 | 32 |
| hbglobin intron2 | 1419 | 347 |
| hbglobin exon3 | 1416 | 53 |
| Modulatory | 1262 | 163 |
| Polynucleotide | | |
| hGH PolyA Signal | 1422 | 477 |
| 3′ ITR | 1382 | 141 |

In some embodiments, the AAV particle viral genome comprises SEQ ID NO: 1368 (VOYHT17) which comprises a 5′ inverted terminal repeat (ITR) sequence region and a 3′ ITR sequence region, a MCS sequence region, a CMV enhancer sequence region, a CMV promoter sequence region, two exon sequence regions (ie1 exon 1 and human beta globin (hbglobin) exon 3 or fragments thereof), two intron sequence regions (ie1 intron 1 and hbglobin intron 2 or fragments thereof), a modulatory polynucleotide region, a human growth hormone (hGH) polyadenylation signal sequence region, and a filler sequence region.

In some embodiments, the AAV particle viral genome comprises a 5′ inverted terminal repeat (ITR) sequence region and a 3′ ITR sequence region, a MCS sequence region, a CMV enhancer sequence region, a CMV promoter sequence region, two exon sequence regions (ie1 exon 1 and human beta globin (hbglobin) exon 3 or fragments thereof), two intron sequence regions (ie1 intron 1 and hbglobin intron 2 or fragments thereof), a modulatory polynucleotide region, a human growth hormone (hGH) polyadenylation signal sequence region, and a filler sequence region. A non-limiting example of an ITR to ITR sequence for use in the AAV particles of the present disclosure having all of the sequence modules above are described in Table 30. In Table 30, the sequence identifier or sequence of the sequence region (Region SEQ ID NO) and the length of the sequence region (Region length) arc described as well as the name and sequence identifier of the ITR to ITR sequence (e.g., VOYHT19 (SEQ ID NO: 1370)).

TABLE 30
Sequence Regions in ITR to ITR Sequences

VOYHT19 (SEQ
ID NO: 1370)

| Sequence | Region SEQ | Region |
|---|---|---|
| Regions | ID NO | length |
| 5′ ITR | 1380 | 141 |
| CMV enhancer | 1409 | 303 |
| CMV Promoter | 1412 | 204 |
| Ie1 exon1 | 1415 | 134 |
| Ie1 intron1 | 1418 | 32 |
| hbglobin intron2 | 1419 | 347 |
| hbglobin exon3 | 1416 | 53 |
| Modulatory | 1262 | 163 |
| Polynucleotide | | |
| hGH PolyA Signal | 1389 | 14 |
| MCS | 1422 | 477 |
| 3′ ITR | 1382 | 141 |

In some embodiments, the AAV particle viral genome comprises SEQ ID NO: 1370 (VOYHT19) which comprises a 5′ inverted terminal repeat (ITR) sequence region and a 3′ ITR sequence region, a MCS sequence region, a CMV enhancer sequence region, a CMV promoter sequence region, two exon sequence regions (ie1 exon 1 and human beta globin (hbglobin) exon 3 or fragments thereof), two intron sequence regions (ie1 intron 1 and hbglobin exon 3 or fragments thereof), a modulatory polynucleotide region, a human growth hormone (hGH) polyadenylation signal sequence region, and a filler sequence region.

In some embodiments, the AAV particle viral genome comprises a 5′ inverted terminal repeat (ITR) sequence region and a 3′ ITR sequence region, two MCS sequence regions, an enhancer sequence region, a promoter sequence region, two exon sequence regions, two intron sequence regions, a modulatory polynucleotide region, a polyadenylation signal sequence region, and a filler sequence region.

In some embodiments, the AAV particle viral genome comprises a 5′ inverted terminal repeat (ITR) sequence region and a 3′ ITR sequence region, two MCS sequence regions, a CMV enhancer sequence region, a CMV promoter sequence region, two exon sequence regions (ie1 exon 1 and human beta globin (hbglobin) exon 3 or fragments thereof), two intron sequence regions (ie1 intron 1 and hbglobin exon 3 or fragments thereof), a modulatory polynucleotide region, a human growth hormone (hGH) polyadenylation signal sequence region, and a filler sequence region. A non-limiting example of an ITR to ITR sequence for use in the AAV particles of the present disclosure having all of the sequence modules above are described in Table 31. In Table 31, the sequence identifier or sequence of the sequence region (Region SEQ ID NO) and the length of the sequence region (Region length) are described as well as the name and sequence identifier of the ITR to ITR sequence (e.g., VOYHT18 (SEQ ID NO: 1369)).

TABLE 31
Sequence Regions in ITR to ITR Sequences

VOYHT18 (SEQ
ID NO: 1369)

| Sequence | Region SEQ | Region |
|---|---|---|
| Regions | ID NO | length |
| 5′ ITR | 1380 | 141 |
| MCS | 1387 | 18 |
| CMV enhancer | 1409 | 303 |
| CMV Promoter | 1412 | 204 |
| Ie1 exon1 | 1415 | 134 |
| Ie1 intron1 | 1418 | 32 |
| hbglobin intron2 | 1419 | 347 |
| hbglobin exon3 | 1416 | 53 |
| Modulatory | 1262 | 163 |
| Polynucleotide | | |
| MCS | 1389 | 14 |
| hGH PolyA Signal | 1422 | 477 |
| 3′ ITR | 1382 | 141 |

In some embodiments, the AAV particle viral genome comprises SEQ ID NO: 1369 (VOYHT18) which comprises a 5′ inverted terminal repeat (ITR) sequence region and a 3′ ITR sequence region, two MCS sequence regions, a CMV enhancer sequence region, a CMV promoter sequence region, two exon sequence regions (ie1 exon 1 and human beta globin (hbglobin) exon 3 or fragments thereof), two intron sequence regions (ie1 intron 1 and hbglobin exon 3 or fragments thereof), a modulatory polynucleotide region, a human growth hormone (hGH) polyadenylation signal sequence region, and a filler sequence region.

In some embodiments, the AAV particle viral genome comprises a 5′ inverted terminal repeat (ITR) sequence region and a 3′ ITR sequence region, two MCS sequence regions, a CMV enhancer sequence region, a CMV promoter sequence region, two exon sequence regions (ie1 exon 1 and human beta globin (hbglobin) exon 3 or fragments thereof), two intron sequence regions (ie1 intron 1 and hbglobin exon 3 or fragments thereof), a modulatory polynucleotide region, a human growth hormone (hGH) polyadenylation signal sequence region, and a filler sequence region. A non-limiting example of an ITR to ITR sequence for use in the AAV particles of the present disclosure having all of the sequence modules above are described in Table 32. In Table 32, the sequence identifier or sequence of the sequence region (Region SEQ ID NO) and the length of the sequence region (Region length) are described as well as the name and sequence identifier of the ITR to ITR sequence (e.g., VOYHT20 (SEQ ID NO: 1371)).

TABLE 32
Sequence Regions in ITR to ITR Sequences

VOYHT20 (SEQ
ID NO: 1371)

| Sequence | Region SEQ | Region |
|---|---|---|
| Regions | ID NO | length |
| 5′ ITR | 1380 | 141 |
| MCS | 1385 | 121 |
| CMV enhancer | 1409 | 303 |
| CMV Promoter | 1412 | 204 |
| Ie1 exon1 | 1415 | 134 |
| Ie1 intron1 | 1418 | 32 |
| hbglobin intron2 | 1419 | 347 |
| hbglobin exon3 | 1416 | 53 |
| Modulatory | 1262 | 163 |
| Polynucleotide | | |
| MCS | 1389 | 14 |
| hGH PolyA Signal | 1422 | 477 |
| 3′ ITR | 1382 | 141 |

In some embodiments, the AAV particle viral genome comprises SEQ ID NO: 1371 (VOYHT20) which comprises a 5′ inverted terminal repeat (ITR) sequence region and a 3′ ITR sequence region, two MCS sequence regions, a CMV enhancer sequence region, a CMV promoter sequence region, two exon sequence regions (ie1 exon 1 and human beta globin (hbglobin) exon 3 or fragments thereof), two intron sequence regions (ie1 intron 1 and hbglobin exon 3 or fragments thereof), a modulatory polynucleotide region, a human growth hormone (hGH) polyadenylation signal sequence region, and a filler sequence region.

In some embodiments, the AAV particle viral genome comprises a 5′ inverted terminal repeat (ITR) sequence region and a 3′ ITR sequence region, two MCS sequence regions, a CMV enhancer sequence region, a CMV promoter sequence region, two exon sequence regions (ie1 exon 1 and human beta globin (hbglobin) exon 3 or fragments thereof), two intron sequence regions (ie1 intron 1 and hbglobin exon 3 or fragments thereof), a modulatory polynucleotide region, a human growth hormone (hGH) polyadenylation signal sequence region, and a filler sequence region. Non-limiting examples of ITR to ITR sequences for use in the AAV particles of the present disclosure having all of the sequence modules above are described in Table 33. In Table 33, the sequence identifier or sequence of the sequence region (Region SEQ ID NO) and the length of the sequence region (Region length) are described as well as the name and sequence identifier of the ITR to ITR sequence (e.g., VOYHT21 (SEQ ID NO: 1372)).

TABLE 33
Sequence Regions in ITR to ITR Sequences

| | VOYHT21 (SEQ | | VOYHT22 (SEQ | |
|---|---|---|---|---|
| | ID NO: 1372) | | ID NO: 1373) | |
| Sequence | Region SEQ | Region | Region SEQ | Region |
| Regions | ID NO | length | ID NO | length |
| 5′ ITR | 1380 | 141 | 1380 | 141 |
| MCS | 1387 | 18 | 1387 | 18 |
| CMV enhancer | 1409 | 303 | 1409 | 303 |
| CMV Promoter | 1412 | 204 | 1412 | 204 |
| Ie1 exon1 | 1415 | 134 | 1415 | 134 |
| Ie1 intron1 | 1418 | 32 | 1418 | 32 |
| hbglobin intron2 | 1419 | 347 | 1419 | 347 |
| hbglobin exon3 | 1416 | 53 | 1416 | 53 |
| Modulatory | 1262 | 163 | 1262 | 163 |
| Polynucleotide | | | | |
| MCS | 1389 | 14 | 1389 | 14 |
| hGH PolyA Signal | 1422 | 477 | 1422 | 477 |
| 3′ ITR | 1382 | 141 | 1382 | 141 |

In some embodiments, the AAV particle viral genome comprises SEQ ID NO: 1372 (VOYHT21 ) which comprises a 5′ inverted terminal repeat (ITR) sequence region and a 3′ ITR sequence region, two MCS sequence regions, a CMV enhancer sequence region, a CMV promoter sequence region, two exon sequence regions (ie1 exon 1 and human beta globin (hbglobin) exon 3 or fragments thereof), two intron sequence regions (ie1 intron 1 and hbglobin exon 3 or fragments thereof), a modulatory polynucleotide region, a human growth hormone (hGH) polyadenylation signal sequence region, and a filler sequence region.

In some embodiments, the AAV particle viral genome comprises SEQ ID NO: 1373 (VOYHT22) which comprises a 5′ inverted terminal repeat (ITR) sequence region and a 3′ ITR sequence region, two MCS sequence regions, a CMV enhancer sequence region, a CMV promoter sequence region, two exon sequence regions (ie1 exon 1 and human beta globin (hbglobin) exon 3 or fragments thereof), two intron sequence regions (ie1 intron 1 and hbglobin exon 3 or fragments thereof), a modulatory polynucleotide region, a human growth hormone (hap polyadenylation signal sequence region, and a filler sequence region.

In some embodiments, the AAV particle viral genome comprises a 5′ inverted terminal repeat (ITR) sequence region and a 3′ ITR sequence region, a MCS sequence region, an enhancer sequence region, a promoter sequence region, two exon sequence regions, two intron sequence regions, a modulatory polynucleotide region, a polyadenylation signal sequence region, and two filler sequence regions.

In some embodiments, the AAV particle viral genome comprises a 5′ inverted terminal repeat (ITR) sequence region and a 3′ ITR sequence region, a MCS sequence region, a CMV enhancer sequence region, a CMV promoter sequence region, two exon sequence regions (ie1 exon 1 and human beta globin (hbglobin) exon 3 or fragments thereof), two intron sequence regions (ie1 intron 1 and hbglobin exon 3 or fragments thereof), a modulatory polynucleotide region, a human growth hormone (hGH) polyadenylation signal sequence region, and two filler sequence regions. A non-limiting example of an ITR to ITR sequence for use in the AAV particles of the present disclosure having all of the sequence modules above are described in Table 34. In Table 34, the sequence identifier or sequence of the sequence region (Region SEQ ID NO) and the length of the sequence region (Region length) are described as well as the name and sequence identifier of the ITR to ITR, sequence (e.g., VOYHT23 (SEQ ID NO: 1374)).

TABLE 34
Sequence Regions in ITR to ITR Sequences

VOYHT23 (SEQ
ID NO: 1374)

| Sequence | Region SEQ | Region |
|---|---|---|
| Regions | ID NO | length |
| 5′ ITR | 1380 | 141 |
| CMV enhancer | 1409 | 303 |
| CMV Promoter | 1412 | 204 |
| Ie1 exon1 | 1415 | 134 |
| Ie1 intron1 | 1418 | 32 |
| hbglobin intron2 | 1419 | 347 |
| hbglobin exon3 | 1416 | 53 |
| Modulatory | 1262 | 163 |
| Polynucleotide | | |
| MCS | 1389 | 14 |
| hGH PolyA Signal | 1422 | 477 |
| 3′ ITR | 1382 | 141 |

In some embodiments, the AAV particle viral genome comprises SEQ ID NO: 1374 (VOYHT23) which comprises a 5′ inverted terminal repeat (ITR) sequence region and a 3′ ITR sequence region, a MCS sequence region, a CMV enhancer sequence region, a CMV promoter sequence region, two exon sequence regions (ie1 exon 1 and human beta globin (hbglobin) exon 3 or fragments thereof), two intron sequence regions (ie1 intron 1 and hbglobin exon 3 or fragments thereof), a modulatory polynucleotide region, a human growth hormone (hGH) polyadenylation signal sequence region, and two filler sequence regions.

In some embodiments, the AAV particle viral genome comprises a 5′ inverted terminal repeat (ITR) sequence region and a 3′ ITR sequence region, two MCS sequence regions, an enhancer sequence region, a promoter sequence region, two exon sequence regions, two intron sequence regions, a modulatory polynucleotide region, a polyadenylation signal sequence region, and two filler sequence regions.

In some embodiments, the AAV particle viral genome comprises a 5′ inverted terminal repeat (ITR) sequence region and a 3′ ITR sequence region, two MCS sequence regions, a CMV enhancer sequence region, a CMV promoter sequence region, two exon sequence regions (ie1 exon 1 and human beta globin (hbglobin) exon 3 or fragments thereof), two intron sequence regions (ie1 intron 1 and hbglobin exon 3 or fragments thereof), a modulatory polynucleotide region, a human growth hormone (hGH) polyadenylation signal sequence region, and two filler sequence regions. A non-limiting example of an ITR to ITR sequence for use in the AAV particles of the present disclosure having all of the sequence modules above are described in Table 35. In Table 35, the sequence identifier or sequence of the sequence region (Region SEQ ID NO) and the length of the sequence region (Region length) are described as well as the name and sequence identifier of the ITR to ITR sequence (e.g., VOYHT24 (SEQ ID NO: 1375)).

TABLE 35
Sequence Regions in ITR to ITR Sequences

VOYHT24 (SEQ
ID NO: 1375)

| Sequence | Region SEQ | Region |
|---|---|---|
| Regions | ID NO | length |
| 5′ ITR | 1380 | 141 |
| MCS | 1386 | 73 |
| CMV enhancer | 1409 | 303 |
| CMV Promoter | 1412 | 204 |
| Ie1 exon1 | 1415 | 134 |
| Ie1 intron1 | 1418 | 32 |
| hbglobin intron2 | 1419 | 347 |
| hbglobin exon3 | 1416 | 53 |
| Modulatory | 1262 | 163 |
| Polynucleotide | | |
| MCS | 1389 | 14 |
| hGH PolyA Signal | 1423 | 476 |
| 3′ ITR | 1382 | 141 |

In some embodiments, the AAV particle viral genome comprises SEQ ID NO: 1375 (VOYHT24) which comprises a 5′ inverted terminal repeat (ITR) sequence region and a 3′ ITR sequence region, two MCS sequence regions, a CMV enhancer sequence region, a CMV promoter sequence region, two exon sequence regions (ie1 exon 1 and human beta globin (hbglobin) exon 3 or fragments thereof), two intron sequence regions (ie1 intron 1 and hbglobin exon 3 or fragments thereof), a modulatory polynucleotide region, a human growth hormone (hGH) polyadenylation signal sequence region, and two filler sequence regions.

In some embodiments, the AAV particle viral genome comprises a 5′ inverted terminal repeat (ITR) sequence region and a 3′ ITR sequence region, two MCS sequence regions, an enhancer sequence region, a promoter sequence region, two exon sequence regions, two intron sequence regions, a modulatory polynucleotide region, and a polyadenylation signal sequence region.

In some embodiments, the AAV particle viral genome comprises a 5′ inverted terminal repeat (ITR) sequence region and a 3′ ITR sequence region, two MCS sequence regions, a CMV enhancer sequence region, a CMV promoter sequence region, two exon sequence regions (ie1 exon 1 and human beta globin (hbglobin) exon 3 or fragments thereof), two intron sequence regions (ie1 intron 1 and hbglobin exon 3 or fragments thereof), a modulatory polynucleotide region, and a human growth hormone (hGH) polyadenylation signal sequence region. Non-limiting examples of an ITR to ITR sequences for use in the AAV particles of the present disclosure having all of the sequence modules above are described in Table 36. In Table 36, the sequence identifier or sequence of the sequence region (Region SEQ ID NO) and the length of the sequence region (Region length) are described as well as the name and sequence identifier of the ITR to ITR sequence (e.g., VOYHT25 (SEQ ID NO: 1376)).

TABLE 36
Sequence Regions in ITR to ITR Sequences

| | VOYHT25 (SEQ | | VOYHT26 (SEQ | |
|---|---|---|---|---|
| | ID NO: 1376) | | ID NO: 1377) | |
| Sequence | Region SEQ | Region | Region SEQ | Region |
| Regions | ID NO | length | ID NO | length |
| 5′ ITR | 1380 | 141 | 1380 | 141 |
| MCS | 1387 | 18 | 1387 | 18 |
| CMV enhancer | 1409 | 303 | 1409 | 303 |
| CMV Promoter | 1412 | 204 | 1412 | 204 |
| Ie1 exon1 | 1415 | 134 | 1415 | 134 |
| Ie1 intron1 | 1418 | 32 | 1418 | 32 |
| hbglobin intron2 | 1419 | 347 | 1419 | 347 |
| hbglobin exon3 | 1416 | 53 | 1416 | 53 |
| Modulatory | 1249 | 158 | 1262 | 163 |
| Polynucleotide | | | | |
| MCS | 1389 | 14 | 1389 | 14 |
| hGH PolyA Signal | 1422 | 477 | 1422 | 477 |
| 3′ ITR | 1382 | 141 | 1382 | 141 |

In some embodiments, the AAV particle viral genome comprises SEQ ID NO: 1376 (VOYHT25) which comprises a 5′ inverted terminal repeat (ITR) sequence region and a 3′ ITR sequence region, two MCS sequence regions, a CMV enhancer sequence region, a CMV promoter sequence region, two exon sequence regions (ie1 exon 1 and human beta globin (hbglobin) exon 3 or fragments thereof), two intron sequence regions (ie1 intron 1 and hbglobin exon 3 or fragments thereof), a modulatory polynucleotide region, and a human growth hormone (hGH) polyadenylation signal sequence region.

In some embodiments, the AAV particle viral genome comprises SEQ ID NO: 1377 (VOYHT26) which comprises a 5′ inverted terminal repeat (ITR) sequence region and a 3′ ITR sequence region, two MCS sequence regions, a CMV enhancer sequence region, a CMV promoter sequence region, two exon sequence regions (ie1 exon 1 and human beta globin (hbglobin) exon 3 or fragments thereof), two intron sequence regions (ie1 intron 1 and hbglobin exon 3 or fragments thereof), a modulatory polynucleotide region, and a human growth hormone (hGH) polyadenylation signal sequence region.

In some embodiments, the AAV particle viral genome comprises a 5′ inverted terminal repeat (ITR) sequence region and a 3′ ITR sequence region, a promoter sequence region, and a modulatory polynucleotide region.

In some embodiments, the AAV particle viral genome comprises a 5′ inverted terminal repeat (ITR) sequence region and a 3′ ITR sequence region, an HI promoter sequence region, and a modulatory polynucleotide region. Non-limiting examples of an ITR to ITR sequences for use in the AAV particles of the present disclosure having all of the sequence modules above are described in Table 37. In Table 37, the sequence identifier or sequence of the sequence region (Region SEQ ID NO) and the length of the sequence region (Region length) are described as well as the name and sequence identifier of the ITR to ITR sequence (e.g., VOYHT27 (SEQ ID NO: 1378)).

TABLE 37
Sequence Regions in ITR to ITR Sequences

| | VOYHT27 (SEQ | | VOYHT28 (SEQ | |
|---|---|---|---|---|
| | ID NO: 1378) | | ID NO: 1379) | |
| Sequence | Region SEQ | Region | Region SEQ | Region |
| Regions | ID NO | length | ID NO | length |
| 5′ ITR | 1381 | 105 | 1381 | 105 |
| H1 Promoter | 1413 | 219 | 1413 | 219 |
| Modulatory | 1259 | 260 | 1249 | 158 |
| Polynucleotide | | | | |
| 3′ ITR | 1383 | 130 | 1383 | 130 |

In some embodiments, the AAV particle viral genome comprises SEQ ID NO: 1378 (VOYHT27) which comprises a 5′ inverted terminal repeat (ITR) sequence region and a 3′ ITR sequence region, an H1 promoter sequence region, and a modulatory polynucleotide region.

In some embodiments, the AAV particle viral genome comprises SEQ ID NO: 1379 (VOYHT28) which comprises a 5′ inverted terminal repeat (ITR) sequence region and a 3′ ITR sequence region, an HI promoter sequence region, and a modulatory polynucleotide region.

In some embodiments, the AAV particle viral genome comprises one or two promoter sequence regions, a modulatory polynucleotide sequence region, and a bovine growth hormone (bGH) polyadenylation signal sequence region.

In some embodiments, the AAV particle viral genome comprises a two promoter sequence regions, a modulatory polynucleotide region, and a polyadenylation signal sequence region.

In some embodiments, the AAV particle viral genome comprises a CMV and T7 promoter sequence region, a modulatory polynucleotide region, and a bGH polyadenylation signal sequence region. Non-limiting examples of sequences for use in the AAV particles of the present disclosure having all of the sequence modules above are described in Table 38. In Table 38, the sequence identifier or sequence of the sequence region (Region SEQ ID NO) and the length of the sequence region (Region length) are described as well as the name of the sequence (e.g., VOYHT29).

TABLE 38
Sequence Regions

| | VOYHT29 | | VOYHT30 | | VOYHT31 | | VOYHT32 | |
|---|---|---|---|---|---|---|---|---|
| Sequence | Region SEQ | Region | Region SEQ | Region | Region SEQ | Region | Region SEQ | Region |
| Regions | ID NO | length | ID NO | length | ID NO | length | ID NO | length |
| CMV Promoter | 1411 | 588 | 1411 | 588 | 1411 | 588 | 1411 | 588 |
| T7 promoter | 1414 | 17 | 1414 | 17 | 1414 | 17 | 1414 | 17 |
| Modulatory | 1249 | 158 | 1259 | 260 | 1255 | 158 | 1253 | 260 |
| Polynucleotide | | | | | | | | |
| bGH PolyA Signal | 1421 | 225 | 1421 | 225 | 1421 | 225 | 1421 | 225 |

In some embodiments, the AAV particle viral genome comprises a CMV promoter sequence region (SEQ ID NO: 1411), a T7 promoter sequence region (SEQ ID NO: 1414), a modulatory polynucleotide sequence region (SEQ ID NO: 1249), and a bGH polyadenylation signal sequence region (SEQ ID NO: 1421).

In some embodiments, the AAV particle viral genome comprises a CMV promoter sequence region (SEQ ID NO: 1411), a T7 promoter sequence region (SEQ ID NO: 1414), a modulatory polynucleotide sequence region (SEQ ID NO: 1259), and a bGH polyadenylation signal sequence region (SEQ ID NO: 1421).

In some embodiments, the AAV particle viral genome comprises a CMV promoter sequence region (SEQ ID NO: 1411), a T7 promoter sequence region (SEQ ID NO: 1414), a modulatory polynucleotide sequence region (SEQ ID NO: 1255), and a bGH polyadenylation signal sequence region (SEQ ID NO: 1421).

In some embodiments, the AAV particle viral genome comprises a CMV promoter sequence region (SEQ ID NO: 1411), a 17 promoter sequence region (SEQ ID NO: 1414), a modulatory polynucleotide sequence region (SEQ ID NO: 1253), and a bGH polyadenylation signal sequence region (SEQ ID NO: 1421).

In some embodiments, the AAV particle viral genome comprises one or two promoter sequence regions, and a modulatory polynucleotide sequence region.

In some embodiments, the AAV particle viral genome comprises a CMV and T7 promoter sequence region, and a modulatory polynucleotide region. Non-limiting examples of sequences for use in the AAV particles of the present disclosure having all of the sequence modules above are described in Table 39. In Table 39, the sequence identifier or sequence of the sequence region (Region SEQ ID NO) and the length of the sequence region (Region length) are described as well as the name of the sequence (e.g., VOYHT33).

TABLE 39
Sequence Regions

| | VOYHT33 | | VOYHT34 | |
|---|---|---|---|---|
| Sequence | Region SEQ | Region | Region SEQ | Region |
| Regions | ID NO | length | ID NO | length |
| CMV Promoter | GTTG | 4 | — | — |
| H1 Promoter | 1413 | 219 | 1413 | 219 |
| Modulatory | 1249 | 158 | 1259 | 260 |
| Polynucleotide | | | | |

In some embodiments, the AAV particle viral genome comprises a CMV promoter sequence region (Sequence: GTTG), promoter sequence region (SEQ ID NO: 1413), and a modulatory polynucleotide sequence region (SEQ ID NO: 1249).

In some embodiments, the AAV particle viral genome comprises a H1 promoter sequence region (SEQ ID NO: 1413), and a modulatory polynucleotide sequence region (SEQ ID NO: 1259).

AAV particles may be modified to enhance the efficiency of delivery. Such modified AAV particles comprising the nucleic acid sequence encoding the siRNA molecules of the present disclosure can be packaged efficiently and can be used to successfully infect the target cells at high frequency and with minimal toxicity.

In some embodiments, the AAV particle comprising a nucleic acid sequence encoding the siRNA molecules of the present disclosure may be a human serotype AAV particle. Such human AAV particle may be derived from any known serotype, e.g., from any one of serotypes AAV1-AAV 11. As non-limiting examples, AAV particles may be vectors comprising an AAV1-derived genome in an AAV1-derived capsid; vectors comprising an AAV2-derived genome in an AAV2-derived capsid; vectors comprising an AAV4-derived genome in an AAV4 derived capsid; vectors comprising an AAV6-derived genome in an AAV6 derived capsid or vectors comprising an AAV9-derived genome in an AAV9 derived capsid.

In other embodiments, the AAV particle comprising a nucleic acid sequence for encoding siRNA molecules of the present disclosure may be a pseudotyped hybrid or chimeric AAV particle which contains sequences and/or components originating from at least two different AAV serotypes. Pseudotyped AAV particles may be vectors comprising an AAV genome derived from one AAV serotype and a capsid protein derived at least in part from a different AAV serotype. As non-limiting examples, such pseudotyped AAV particles may be vectors comprising an AAV2-derived genome in an AAV1-derived capsid; or vectors comprising, an AAV2-derived genome in an AAV6-derived capsid; or vectors comprising an AAV2-derived genome in an AAV4-derived capsid; or an AAV2-derived genome in an AAV9-derived capsid. In like fashion, the present disclosure contemplates any hybrid or chimeric AAV particle.

In other embodiments, AAV particles comprising a nucleic acid sequence encoding the siRNA molecules of the present disclosure may be used to deliver siRNA molecules to the central nervous system (e.g., U.S. Pat. No. 6,180,613; the contents of which is herein incorporated by reference in its entirety).

In some aspects, the AAV particles comprising a nucleic acid sequence encoding the siRNA molecules of the present disclosure may further comprise a modified capsid including peptides from non-viral origin. In other aspects, the AAV particle may contain a CNS specific chimeric capsid to facilitate the delivery of encoded siRNA. duplexes into the brain and the spinal cord. For example, an alignment of cap nucleotide sequences from AAV variants exhibiting CNS tropism may be constructed to identify variable region (VR) sequence and structure.

In any of the DNA and RNA sequences referenced and/or described herein, the single letter symbol has the following description: A for adenine; C for cytosine; G for guanine; T for thymine; U for Uracil; W for weak bases such as adenine or thymine; S for strong nucleotides such as cytosine and guanine; M for amino nucleotides such as adenine and cytosine; K for keto nucleotides such as guanine and thymine; R for purines adenine and guanine; Y for pyrimidine cytosine and thymine; B for any base that is not A (e.g., cytosine, guanine, and thymine); D for any base that is not C (e.g., adenine, guanine, and thymine); H for any base that is not G (e.g., adenine, cytosine, and thymine); V for any base that is not T (e.g., adenine, cytosine, and guanine); N for any nucleotide (which is not a gap); and Z is for zero. In any of the amino acid sequences referenced and/or described herein, the single letter symbol has the following description: G (Gly) for Glycine; A (Ala) for Alanine; L (Leu) for Leucine; M (Met) for Methionine; F (Phe) for Phenylalanine; W (Trp) for Tryptophan; K (Lys) for Lysine; Q (Gln) for Glutamine; E (Glu) for Glutamic Acid; S (Ser) for Serine; P (Pro) for Prolific; V (Val) for Valine; I (Ile) for Isoleucine; C (Cys) for Cysteine; Y (Tyr) for Tyrosine; H (His) for Histidine; R (Arg) for Arginine; N (Asn) for Asparagine; D (Asp) for Aspartic Acid; T (Thr) for Threonine; B (Asx) for Aspartic acid or Asparagine; J (Xle) for Leucine or Isoleucine; O (Pyl) for Pyrrolysine; U (Sec) for Selenocysteine; X (Xaa) for any amino acid; and Z (Glx) for Glutamine or Glutamic acid.

### Viral Production

The present disclosure provides a method for the generation of parvoviral particles, e.g. AAV particles, by viral genome replication in a viral replication cell comprising contacting the viral replication cell with an AAV polynucleotide or AAV genome.

The present disclosure provides a method for producing an AAV particle having enhanced (increased, improved) transduction efficiency comprising the steps of: 1) co-transfecting competent bacterial cells with a bacmid vector and either a viral construct vector and/or AAV payload construct vector, 2) isolating the resultant viral construct expression vector and AAV payload construct expression vector and separately transfecting viral replication cells, 3) isolating and purifying resultant payload and viral construct particles comprising viral construct expression vector or AAV payload construct expression vector, 4) co-infecting a viral replication cell with both the AAV payload and viral construct particles comprising viral construct expression vector or AAV payload construct expression vector, and 5) harvesting and purifying the viral particle comprising a parvoviral genome.

In some embodiments, the present disclosure provides a method for producing an AAV particle comprising the steps of 1) simultaneously co-transfecting mammalian cells, such as, but not limited to HEK.293 cells, with a payload region, a construct expressing rep and cap genes and a helper construct, 2) harvesting and purifying the AAV particle comprising a viral genome.

### Cells

106401 The present disclosure provides a cell comprising an AAV polynucleotide and/or AAV genome.

Viral production disclosed herein describes processes and methods for producing AAV particles that contact a target cell to deliver a payload construct, e.g. a recombinant viral construct, which comprises a polynucleotide sequence encoding a payload molecule.

In some embodiments, the AAV particles may be produced in a viral replication cell that comprises an insect cell.

Growing conditions for insect cells in culture, and production of heterologous products in insect cells in culture are well-known in the art, see U.S. Pat. No. 6,204,059, the contents of which are herein incorporated by reference in their entirety.

Any insect cell which allows for replication of parvovirus and which can be maintained in culture can be used in accordance with the present disclosure. Cell lines may be used from Spodoptera frugiperda, including, but not limited to the Sf9 or Sf21 cell lines, Drosophila cell lines, or mosquito cell lines, such as Aedes albopictus derived cell lines. Use of insect cells for expression of heterologous proteins is well documented, as are methods of introducing nucleic acids, such as vectors, e.g., insect-cell compatible vectors, into such cells and methods of maintaining such cells in culture. See, for example, Methods in Molecular Biology, ed. Richard, Humana Press, NJ (1995); O'Reilly et al., Baculovirus Expression Vectors, A Laboratory Manual, Oxford Univ. Press (1994); Samulski et al., J. Vir.63:3822-8 (1989); Kajigaya et al., Proc. Nat'l. Acad. Set. USA 88: 4646-50 (1991); Ruffing et al., J. Vir. 66:6922-30 (1992); Kimbauer et al., Vir.219:37-44 (1996); Zhao et al., Vir.272:382-93 (2000); and Samulski et al., U.S. Pat. No. 6,204,059, the contents of each of which is herein incorporated by reference in its entirety.

The viral replication cell may be selected from any biological organism, including prokaryotic (e.g., bacterial) cells, and eukaryotic cells, including, insect cells, yeast cells and mammalian cells. Viral replication cells may comprise mammalian cells such as A549, WEH1, 3T3, 10T1/2, BHK, MDCK, COS 1, COS 7, BSC 1. BSC 40, BMT 10, VERO. W138, HeLa. HEK293, Saos, C2C12, L cells, HT1080, HepG2 and primary fibroblast, hepatocyte and myoblast cells derived from mammals. Viral replication cells comprise cells derived from mammalian species including, but not limited to, human, monkey, mouse, rat, rabbit, and hamster or cell type, including but not limited to fibroblast, hepatocyte, tumor cell, cell line transformed cell, etc.

### Small Scale Production of AAV Particles

Viral production disclosed herein describes processes and methods for producing AAV particles that contact a target cell to deliver a payload, e.g. a recombinant viral construct, which comprises a polynucleotide sequence encoding a payload.

In some embodiments, the AAV particles may be produced in a viral replication cell that comprises a mammalian cell.

Viral replication cells commonly used for production of recombinant AAV particles include, but are not limited to 293 cells, COS cells, HeLa cells, KB cells, and other mammalian cell lines as described in U.S. Pat. Nos. 6,156,303, 5,387,484, 5,741,683, 5,691,176, and 5,688,676; U.S. patent application 2002/0081721, and International Patent Applications WO 00/47757, WO 00/24916, and WO 96/17947, the contents of each of which are herein incorporated by reference in their entireties.

In some embodiments, AAV particles are produced in mammalian-cells wherein all three VP proteins are expressed at a stoichiometry approaching 1:1:10 (VP1:VP2:VP3). The regulatory mechanisms that allow this controlled level of expression include the production of two mRNAs, one for VP1, and the other for VP2 and VP3, produced by differential splicing.

In another embodiment. AAV particles are produced in mammalian cells using a triple transfection method wherein a payload construct, parvoviral Rep and parvoviral Cap and a helper construct are comprised within three different constructs. The triple transfection method of the three components of AAV particle production may be utilized to produce small lots of virus for assays including transduction efficiency, target tissue (tropism) evaluation, and stability.

AAV particles may be produced by triple transfection or baculovirus mediated virus production, or any other method known in the art. Any suitable permissive or packaging cell known in the art may be employed to produce the vectors. Mammalian cells are often preferred. Also preferred are trans-complementing packaging cell lines that provide functions deleted from a replication-defective helper virus, e.g., 293 cells or other E1a trans-complementing cells.

The gene cassette may contain some or all of the parvovinis (e.g., AAV) cap and rep genes. Preferably, however, some or all of the cap and rep functions are provided in trans by introducing a packaging vector(s) encoding the capsid and/or Rep proteins into the cell. Most preferably, the gene cassette does not encode the capsid or Rep proteins. Alternatively, a packaging cell line is used that is stably transformed to express the cap and/or rep genes.

Recombinant AAV virus particles are, in some cases, produced and purified from culture supernatants according to the procedure as described in US20160032254, the contents of which are incorporated by reference. Production may also involve methods known in the art including those using 293T cells, sf9 insect cells, triple transfection or any suitable production method.

In some cases, 293T cells (adhesion/suspension) are transfected with polyethyleneimine (PEI) with plasmids required for production of AAV, i.e., AAV2 rep, an adenoviral helper construct and a ITR flanked transgene cassette. The AAV2 rep plasmid also contains the cap sequence of the particular virus being studied. Twenty-four hours after transfection (no medium changes for suspension), which occurs in DMEM/F17 with/without serum, the medium is replaced with fresh medium with or without serum. Three (3) days after transfection, a sample is taken from the culture medium of the 293 adherent cells. Subsequently cells are scraped, or suspension cells are pelleted, and transferred into a receptacle. For adhesion cells, after centrifugation to remove cellular pellet, a second sample is taken from the supernatant after scraping. Next, cell lysis is achieved by three consecutive freeze-thaw cycles (˜80C to 37C) or adding detergent triton. Cellular debris is removed by centrifugation or depth filtration and sample 3 is taken from the medium. The samples are quantified for AAV particles by RNase resistant genome titration by DNA qPCR, The total production yield from such a transfection is equal to the particle concentration from sample 3.

AAV particle titers are measured according to genome copy number (genome particles per milliliter). Genome particle concentrations are based on DNA qPCR of the vector DNA as previously reported (Clark et al. (1999) Hum. Gene Ther., 10:1031-1039; Veldwijk et al, (2002) Mol. Ther., 6:272-278).

### Baculovirus

Particle production disclosed herein describes processes and methods for producing AAV particles that contact a target cell to deliver a payload construct which comprises a polynucleotide sequence encoding a payload.

Briefly, the viral construct vector and the AAV payload construct vector are each incorporated by a transposondonor/acceptor system into a bacmid, also known as a baculovirus plasmid, by standard molecular biology techniques known and performed by a person skilled in the art. Transfection of separate viral replication cell populations produces two baculoviruses, one that comprises the viral construct expression vector, and another that comprises the AAV payload construct expression vector, The two baculoviruses may be used to infect a single viral replication cell population for production of AAV particles,

Baculovirus expression vectors for producing viral particles in insect cells, including but not limited to Spodoptera frugiperda (Sf9) cells, provide high titers of viral particle product, Recombinant baculovirus encoding the viral construct expression vector and AAV payload construct expression vector initiates a productive infection of viral replicating cells. Infectious baculovirus particles released from the primary infection secondarily infect additional cells in the culture, exponentially infecting the entire cell culture population in a number of infection cycles that is a function of the initial multiplicity of infection, see Urabe, M. et al,. J Virol, 2006 February; 80 (4):1874-85, the contents of which are herein incorporated by reference in their entirety.

Production of AAV particles with baculovirus in an insect cell system may address known baculovirus genetic and physical instability. In some embodiments, the production system addresses baculovirus instability over multiple passages by utilizing a titerless infected-cells preservation and scale-up system. Small scale seed cultures of viral producing cells are transfected with viral expression constructs encoding the structural, non-structural, components of the viral particle. Baculovirus-infected viral producing cells are harvested into aliquots that may be cryopreserved in liquid nitrogen; the aliquots retain viability and infectivity for infection of large scale viral producing cell culture Wasilko D J et al., Protein Expr Purif. 2009 June; 65(2):122-32, the contents of which are herein incorporated by reference in their entirety.

A genetically stable baculovirus may be used to produce source of the one or more of the components for producing AAV particles in invertebrate cells. In some embodiments, defective baculovirus expression vectors may be maintained episomally in insect cells. In such an embodiment the bacmid vector is engineered with replication control elements, including but not limited to promoters, enhancers, and/or cell-cycle regulated replication elements.

In some embodiments, baculoviruses may be engineered with a (non-) selectable marker for recombination into the chitinase/cathepsin locus. The chia/v-cath locus is non-essential for propagating baculovirus in tissue culture, and the V-cath (EC 3.4.22.50) is a cysteine endoprotease that is most active on Arg-Arg dipeptide containing substrates. The Arg-Arg dipeptide is present in densovirus and parvovirus capsid structural proteins but infrequently occurs in dependovirus VP1.

In some embodiments, stable viral replication cells permissive for baculovirus infection are engineered with at least one stable integrated copy of any of the elements necessary for AAV replication and viral particle production including, but not limited to, the entire AAV genome, Rep and Cap genes, Rep genes, Cap genes, each Rep protein as a separate transcription cassette, each VP protein as a separate transcription cassette. the AAP (assembly activation protein), or at least one of the baculovirus helper genes with native or non-native promoters. Large-Scale Production

In some embodiments, AAV particle production may be modified to increase the scale of production. Large scale viral production methods according to the present disclosure may include any of those taught in U.S. Pat. Nos. 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508 or International Publication Nos. WO1996039530, WO1998010088, WO1999014354, WO1999015685, WO1999047691, WO2000055342, WO2000075353 and WO2001023597, the contents of each of which are herein incorporated by reference in their entirety. Methods of increasing viral particle production scale typically comprise increasing the number of viral replication cells. In some embodiments, viral replication cells comprise adherent cells. To increase the scale of viral particle production by adherent viral replication cells, larger cell culture surfaces are required. In some cases, large-scale production methods comprise the use of roller bottles to increase cell culture surfaces. Other cell culture substrates with increased surface areas are known in the art. Examples of additional adherent cell culture products with increased surface areas include, but are not limited to CELLSTACK®, CELLCUBE® (Corning Corp., Corning, N.Y.) and NUNC™ CELL FACTORY™ (Thermo Scientific. Waltham, Mass.) In some cases, large-scale adherent cell surfaces may comprise from about 1,000 cm² to about 100,000 cm². In some cases, large-scale adherent cell cultures may comprise from about 10⁷ to about 10⁹ cells, from about 10⁸ to about 10¹⁰ cells, from about 10⁹ to about 10¹² cells or at least 10¹² cells. In some cases, large-scale adherent cultures may produce from about 10⁹ to about 10¹², from about 10¹⁰ to about 10¹³, from about 10¹¹ to about 10¹⁴, from about 10¹² to about 10¹⁵ or at least 10¹⁵ viral particles.

In some embodiments, large-scale viral production methods of the present disclosure may comprise the use of suspension cell cultures. Suspension cell culture allows for significantly increased numbers of cells. Typically, the number of adherent cells that can be grown on about 10-50 cm² of surface area can be grown in about 1 cm³ volume in suspension.

Transfection of replication cells in large-scale culture formats may be carried out according to any methods known in the art. For large-scale adherent cell cultures, transfection methods may include, but are not limited to the use of inorganic compounds (e.g. calcium phosphate), organic compounds [e.g. polyethyleneimine (PEI)] or the use of non-chemical methods (e.g. electroporation.) With cells grown in suspension, transfection methods may include, but are not limited to the use of calcium phosphate and the use of PEI. In some cases, transfection of large-scale suspension cultures may be carried out according to the section entitled “Transfection Procedure” described in Feng, L. et al., 2008, Biotechnol Appl. Biochem. 50:121-32, the contents of which are herein incorporated by reference in their entirety. According to such embodiments, PEI-DNA complexes may be formed for introduction of plasmids to be transfected. In some cases, cells being transfected with PEI-DNA complexes may be ‘shocked’ prior to transfection. This comprises lowering cell culture temperatures to 4° C. for a period of about 1 hour. In some cases, cell cultures may be shocked for a period of from about 10 minutes to about 5 hours. In some cases, cell cultures may be shocked at a temperature of from about 0° C. to about 20° C.

In some cases, transfections may include one or more vectors for expression of an RNA effector molecule to reduce expression of nucleic acids from one or more AAV payload construct. Such methods may enhance the production of viral particles by reducing cellular resources wasted on expressing payload constructs. In some cases, such methods may be carried according to those taught in US Publication No. US2014/0099666, the contents of which are herein incorporated by reference in their entirety.

### Bioreactors

In some embodiments, cell culture bioreactors may be used for large scale viral production. In some cases, bioreactors comprise stirred tank reactors. Such reactors generally comprise a vessel, typically cylindrical in shape, with a stirrer (e.g. impeller.) In some embodiments, such bioreactor vessels may be placed within a water jacket to control vessel temperature and/or to minimize effects from ambient temperature changes. Bioreactor vessel volume may range in size from about 500 ml to about 2 L, from about 1 L to about 5 L, from about 2.5 L to about 20 L, from about 10 L to about 50 L, from about 25 L to about 100 L, from about 75 L to about 500 L, from about 250 L to about 2,000 L, from about 1,000 L to about 10,000 L, from about 5,000 L to about 50,000 L or at least 50,000 L. Vessel bottoms may be rounded or flat. In some cases, animal cell cultures may be maintained in bioreactors with rounded vessel bottoms.

In some cases, bioreactor vessels may be warmed through the use of a thermocirculator. Thermocirculators pump heated water around water jackets. In some cases, heated water may be pumped through pipes (e.g coiled pipes) that are present within bioreactor vessels. in some cases, warm air may be circulated around bioreactors, including, but not limited to air space directly above culture medium. Additionally, pH and CO₂ levels may be maintained to optimize cell viability.

In some cases, bioreactors may comprise hollow-fiber reactors. Hollow-fiber bioreactors may support the culture of both anchorage dependent and anchorage independent cells. Further bioreactors may include, but are not limited to, packed-bed or fixed-bed bioreactors. Such bioreactors may comprise vessels with glass beads for adherent cell attachment. Further packed-bed reactors may comprise ceramic beads,

In some cases, viral particles are produced through the use of a disposable bioreactor. In some embodiments, such bioreactors may include WAVE™ disposable bioreactors.

In some embodiments, AAV particle production in animal cell bioreactor cultures may be carried out according to the methods taught in U.S. Pat. Nos. 5,064764, 6,194,191, 6,566,118, 8,137,948 or US Patent Application No. US2011/0229971, the contents of each of which are herein incorporated by reference in their entirety.

### Cell Lysis

Cells of the disclosure, including, but not limited to viral production cells, may be subjected to cell lysis according to any methods known. Cell lysis may be carried out to obtain one or more agents (e.g, viral particles) present within any cells of the disclosure. In some embodiments, cell lysis may be carried out according to any of the methods listed in U.S. Pat. Nos. 7,326,555, 7,579,181, 7,048,920, 6,410,300, 6,436,394, 7,732,129, 7,510,875, 7,445,930, 6,726,907, 6,194,191, 7,125,706, 6,995,006, 6,676,935, 7,968,333, 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508 or International Publication Nos. WO1996039530, WO1998010088, WO1999014354, WO1999015685, WO1999047691, WO7000055342, WO2000075353 and WO2001023597, the contents of each of which are herein incorporated by reference in their entirety. Cell lysis methods may be chemical or mechanical. Chemical cell lysis typically comprises contacting one or more cells with one or more lysis agent. Mechanical lysis typically comprises subjecting one or more cells to one or more lysis condition and/or one or more lysis force.

In some embodiments, chemical lysis may be used to lyse cells. As used herein, the term “lysis agent” refers to any agent that may aid in the disruption of a cell. In some cases, lysis agents are introduced in solutions, termed lysis solutions or lysis buffers. As used herein, the term “lysis solution” refers to a solution (typically aqueous) comprising one or more lysis agent. In addition to lysis agents, lysis solutions may include one or more buffering agents, solubilizing agents, surfactants, preservatives, cryoprotectants, enzymes, enzyme inhibitors and/or chelators. Lysis buffers are lysis solutions comprising one or more buffering agent. Additional components of lysis solutions may include one or more solubilizing agent. As used herein, the term “solubilizing agent” refers to a compound that enhances the solubility of one or more components of a solution and/or the solubility of one or more entities to which solutions are applied. In some cases, solubilizing agents enhance protein solubility. In some cases, solubilizing agents are selected based on their ability to enhance protein solubility while maintaining protein conformation and/or activity.

Exemplary lysis agents may include any of those described in U.S. Pat. Nos. 8,685,734, 7,901,921, 7,732,129, 7,223,585, 7,125,706, 8,236,495, 8,110,351, 7,419,956, 7,300,797, 6,699,706 and 6,143,567, the contents of each of which are herein incorporated by reference in their entirety. In some cases, lysis aunts may be selected from lysis salts, amphoteric agents, cationic agents, ionic detergents and non-ionic detergents. Lysis salts may include, but are not limited to, sodium chloride (NaCl) and potassium chloride (KCl) Further lysis salts may include any of those described in U.S. Pat. Nos. 8,614,101, 7,326,555, 7,579,181, 7,048,920, 6,410,300, 6,436,394, 7,732,129, 7,510,875, 7,445,930, 6,726,907, 6,194,191, 7,125,706, 6,995,006, 6,676,935 and 7,968,333, the contents of each of which are herein incorporated by reference in their entirety. Concentrations of salts may be increased or decreased to obtain an effective concentration for rupture of cell membranes. Amphoteric agents, as referred to herein, are compounds capable of reacting as an acid or a base. Amphoteric agents may include, but are not limited to lysophosphatidylcholine, 3((3-Cholamidopropyl) dimethylammonium)-1-propanesulfonate (CHAPS), ZWITTERGENT® and the like. Cationic agents may include, but are not limited to, cetyltrimethylammonium bromide (C (16) TAB) and Benzalkonium chloride. Lysis agents comprising detergents may include ionic detergents or non-ionic detergents. Detergents may function to break apart or dissolve cell structures including, but not limited to cell membranes, cell walls, lipids, carbohydrates, lipoproteins and glycoproteins. Exemplary ionic detergents include any of those taught in U.S. Pat. Nos. 7,625,570 and 6,593,123 or US Publication No. US:2014/0087361, the contents of each of which are herein incorporated by reference in their entirety. Some ionic detergents may include, but are not limited to, sodium dodecyl sulfate (SDS), cholate and deoxycholate. In some cases, ionic detergents may be included in lysis solutions as a solubilizing agent. Non-ionic detergents may include, but are not limited to octylglucoside, digitonin, lubrol, C12E8, TWEEN®-20, TWEEN®-80, Triton X-100 and Noniodet P-40. Non-ionic detergents are typically weaker lysis agents but may be included as solubilizing agents for solubilizing cellular and/or viral proteins. Further lysis agents may include enzymes and urea. In some cases, one or more lysis agents may he combined in a lysis solution in order to enhance one or more of cell lysis and protein solubility. In some cases, enzyme inhibitors may be included in lysis solutions in order to prevent proteolysis that may be triggered by cell membrane disruption.

In some embodiments, mechanical cell lysis is carried out. Mechanical cell lysis methods may include the use of one or more lysis condition and/or one or more lysis force. As used herein, the term “lysis condition” refers to a state or circumstance that promotes cellular disruption. Lysis conditions may comprise certain temperatures, pressures, osmotic purity, salinity and the like. In some cases, lysis conditions comprise increased or decreased temperatures. According to some embodiments, lysis conditions comprise changes in temperature to promote cellular disruption. Cell lysis carried out according to such embodiments may include freeze-thaw lysis. As used herein, the term “freeze-thaw lysis” refers to cellular lysis in which a cell solution is subjected to one or more freeze-thaw cycle. According to freeze-thaw lysis methods, cells in solution are frozen to induce a mechanical disruption of cellular membranes caused by the formation and expansion of ice crystals. Cell solutions used according freeze-thaw lysis methods, may further comprise one or more lysis agents, solubilizing agents, buffering agents, cryoprotectants, surfactants, preservatives, enzymes, enzyme inhibitors and/or dictators. Once cell solutions subjected to freezing are thawed, such components may enhance the recovery of desired cellular products. In some cases, one or more cryoprotectants are included in cell solutions undergoing freeze-thaw lysis. As used herein, the term “cryoprotectant” refers to an agent used to protect one or more substance from damage due to freezing. Cryoprotectants may include any of those taught in US Publication No. US2013/0323302 or U.S. Pat. Nos. 6,503,888, 6,180,613, 7,888,096, 7,091,030, the contents of each of which are herein incorporated by reference in their entirety. In some cases, cryoprotectants may include, but are not limited to dimethyl sulfoxide, 1,2-propanediol, 2,3-butanediol, formamide, glycerol, ethylene glyco, 1,3-propanediol and n-dimethyl formamide, polyvinylpyrrolidone, hydroxyethyl starch, agarose, dextrans, inositol, glucose, hydroxyethylstarch, lactose, sorbitol, methyl glucose, sucrose and urea. In some embodiments, freeze-thaw lysis may be carried out according to any of the methods described in U.S. Pat. No. 7,704,721, the contents of which are herein incorporated by reference in their entirety. As used herein, the term “lysis force” refers to a physical activity used to disrupt a cell. Lysis forces may include, but are not limited to mechanical forces, some forces, gravitational forces, optical forces, electrical forces and the like. Cell lysis carried out by mechanical force is referred to herein as “mechanical lysis.” Mechanical forces that may be used according to mechanical lysis may include high shear fluid forces. According to such methods of mechanical lysis, a microfluidizer may be used. Microfluidizers typically comprise an inlet reservoir where cell solutions may be applied. Cell solutions may then be pumped into an interaction chamber via a pump (e.g. high-pressure pump) at high speed and/or pressure to produce shear fluid forces. Resulting lysates may then be collected in one or more output reservoir. Pump speed and/or pressure may be adjusted to modulate cell lysis and enhance recovery of products (e.g. viral particles.) Other mechanical lysis methods may include physical disruption of cells by scraping.

Cell lysis methods may be selected based on the cell culture format of cells to be lysed. For example, with adherent cell cultures, some chemical and mechanical lysis methods may be used. Such mechanical lysis methods may include freeze-thaw lysis or scraping. In another example, chemical lysis of adherent cell cultures may be carried out through incubation with lysis solutions comprising surfactant, such as Triton-X-100. In some cases, cell lysates generated from adherent cell cultures may be treated with one more nuclease to lower the viscosity of the lysates caused by liberated DNA.

In some embodiments, a method for harvesting AAV particles without lysis may be used for efficient and scalable AAV particle production. In a non-limiting example, AAV particles may be produced by culturing an AAV particle lacking a heparin binding site, thereby allowing the AAV particle to pass into the supernatant, in a cell culture, collecting supernatant from the culture; and isolating the AAV particle from the supernatant, as described in US Patent Application 20090275107, the contents of which are incorporated herein by reference in their entirety.

### Clarification

Cell lysates comprising viral particles may be subjected to clarification. Clarification refers to initial steps taken in purification of viral particles from cell lysates. Clarification serves to prepare lysates for further purification by removing larger, insoluble debris. Clarification steps may include, but are not limited to, centrifugation and filtration. During clarification, centrifugation may be carried out at low speeds to remove larger debris only. Similarly, filtration may be carried out using filters with larger pore sizes so that only larger debris is removed. In some cases, tangential flow filtration may be used during clarification. Objectives of viral clarification include high throughput processing of cell lysates and to optimize ultimate viral recovery. Advantages of including a clarification step include scalability for processing of larger volumes of lysate. In some embodiments, clarification may be carried out according to any of the methods presented in U.S. Pat. Nos. 8,524,446, 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498, 7,491,508, US Publication Nos. US2013/0045186, US2011/0263027, US2011/0151434, US2003/0138772, and International Publication Nos. WO2002012455, WO1996039530, WO1998010088, WO1999014354, WO1999015685, WO1999047691, WO2000055342, WO2000075353 and WO2001023597, the contents of each of which are herein incorporated by reference in their entirety.

Methods of cell lysate clarification by filtration are well understood in the art and may he carried out according to a variety of available methods including, but not limited to passive filtration and flow filtration. Filters used may comprise a variety of materials and pore sizes. For example, cell lysate filters may comprise pore sizes of from about 104 to about 5 μM, from about 0.5 μM to about 2 μM, from about 0.1 μM to about 1 μM, from about 0.05 μM to about 0.05 μM and from about 0.001 μM to about 0.1 μM. Exemplary pore sizes for cell lysate filters may include, but are not limited to, 2.0, 1.9, 1.8, 1.7, 1.6, 1.5, 1.4, 1.3, 1.2, 1.1, 1, 0.9, 0.8, 0.7, 0.6, 0.5, 0.4, 0.3, 0.2, 0.1, 0.95, 0.9, 0.85, 0.8, 0.75, 0.7, 0.65, 0.6, 0.55, 0.5, 0.45, 0.4, 0.35, 0.3, 0.25, 0.2, 0.15, 0.1, 0.05, 0.22, 0.21, 0.20, 0.19, 0.18, 0.17, 0.16, 0.15, 0.14, 0.13, 0.12, 0.11, 0.1, 0.09, 0.08, 0.07, 0.06, 0.05, 0.04, 0.03, 0.02, 0.01, 0.02, 0.019, 0.018, 0.017, 0.016, 0.015, 0.014, 0.013, 0.012, 0.011, 0.01, 0.009, 0.008, 0.007, 0.006, 0.005, 0.004, 0.003, 0.002, 0.001 and 0.001 μM In some embodiments, clarification may comprise filtration through a filter with 2.0 μM pore size to remove large debris, followed by passage through a filter with 0.45 μM pore size to remove intact cells.

Filter materials may be composed of a variety of materials. Such materials may include, but are not limited to, polymeric materials and metal materials (e.g. sintered metal and pored aluminum.) Exemplary materials may include, but are not limited to nylon, cellulose materials (e.g. cellulose acetate), polyvinylidene fluoride (PVDF), polyethersulfone, polyamide, polysulfone, polypropylene, and polyethylene terephthalate. In some cases, filters useful for clarification of cell lysates may include, but are not limited to ULTIPLEAT PROFILE™ filters (Pall Corporation. Port Washington, N.Y.), SUPOR™ membrane filters (Pall Corporation, Port Washington, N.Y.)

In some cases, flow filtration may be carried out to increase filtration speed and/or effectiveness. In some cases, flow filtration may comprise vacuum filtration. According to such methods, a vacuum is created on the side of the filter opposite that of cell lysate to be filtered. In some cases, cell lysates may be passed through filters by centrifugal forces. In some cases, a pump is used to force cell lysate through clarification filters. Flow rate of cell lysate through one or more filters may be modulated by adjusting one of channel size and/or fluid pressure.

According to some embodiments, cell lysates may be clarified by centrifugation. Centrifugation may be used to pellet insoluble particles in the lysate. During clarification, centrifugation strength [expressed in terms of gravitational units (g), which represents multiples of standard gravitational force] may be lower than in subsequent purification steps. In some cases, centrifugation may be carried out on cell lysates at from about 200 g to about 800 g, from about 500 g to about 1500 g, from about 1000 g to about 5000 g, from about 1200 g to about 10000 g or from about 8000 g to about 15000 g. In some embodiments, cell lysate centrifugation is carried out at 8000 g for 15 minutes. In some cases, density gradient centrifugation may be carried out in order to partition particulates in the cell lysate by sedimentation rate. Gradients used according to methods of the present disclosure may include, but are not limited to, cesium chloride gradients and iodixanol step gradients.

### Purification: Chromatography

In some cases, AAV particles may be purified from clarified cell lysates by one or more methods of chromatography. Chromatography refers to any number of methods known in the art for separating out one or more elements from a mixture. Such methods may include, but are not limited to, ion exchange chromatography (e.g. cation exchange chromatography and anion exchange chromatography), immunoaffinity chromatography and size-exclusion chromatography. In some embodiments, methods of viral chromatography may include any of those taught in U.S. Pat. Nos. 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508 or International Publication Nos. WO1996039530, WO1998010088, WO1999014354, WO1999015685, WO1999047691, WO2000055342, WO2000075353 and WO2001023597, the contents of each of which are herein incorporated by reference in their entirety.

In some embodiments, ion exchange chromatography may be used to isolate viral particles. Ion exchange chromatography is used to bind viral particles based on charge-charge interactions between capsid proteins and charged sites present on a stationary phase, typically a column through which viral preparations (e.g. clarified lysates) are passed. After application of viral preparations, bound viral particles may then be eluted by applying an elution solution to disrupt the charge-charge interactions. Elution solutions may be optimized by adjusting salt concentration and/or pH to enhance recovery of hound viral particles. Depending on the charge of viral capsids being isolated, cation or anion exchange chromatography methods may be selected. Methods of ion exchange chromatography may include, but are not limited to, any of those taught in U.S. Pat. Nos. 7,419,817, 6,143,548, 7,094,604, 6,593,123, 7,015,026 and 8,137,948, the contents of each of which are herein incorporated by reference in their entirety. In some embodiments, immunoaffinity chromatography may be used. Immunoaffinity chromatography is a form of chromatography that utilizes one or more immune compounds (e.g. antibodies or antibody-related structures) to retain viral particles. Immune compounds may bind specifically to one or more structures on viral particle surfaces, including, but not limited to one or more viral coat protein. In some cases, immune compounds may be specific for a particular viral variant. In some cases, immune compounds may bind to multiple viral variants. In some embodiments, immune compounds may include recombinant single-chain antibodies. Such recombinant single chain antibodies may include those described in Smith, R. H. et al., 2009. Mol. Ther. 17(11):1888-96, the contents of which are herein incorporated by reference in their entirety. Such immune compounds are capable of binding to several AAV capsid variants, including, but not limited to AAV1, AAV2, AAV6 and AAV8.

In some embodiments, size-exclusion chromatography (SEC) may be used. SEC may comprise the use of a gel to separate particles according to size. In viral particle purification, SEC filtration is sometimes referred to as “polishing.” In some cases, SEC may be carried out to generate a final product that is near-homogenous. Such final products may in some cases be used in pre-clinical studies and/or clinical studies (Kotin, R. M. 2011. Human Molecular Genetics. 20(1):R2-R6, the contents of which are herein incorporated by reference in their entirety.) In some cases, SEC may be carried out according to any of the methods taught in U.S. Pat. Nos. 6,143,548, 7,015,026, 8,476,418, 6,410,300, 8,476,418, 7,419,817, 7,094,604, 6,593,123, and 8,137,948, the contents of each of which are herein incorporated by reference in their entirety.

In some embodiments, the compositions comprising at least one AAV particle may be isolated or purified using the methods described in U.S. Pat. No. 6,146,874, the contents of which are herein incorporated by reference in its entirety.

In some embodiments, the compositions comprising at least one AAV particle may be isolated or purified using the methods described in U.S. Pat. No. 6,660,514, the contents of which are herein incorporated by reference in its entirety.

In some embodiments, the compositions comprising at least one AAV particle may be isolated or purified using the methods described in U.S. Pat. No. 8,283,151, the contents of which are herein incorporated by reference in its entirety.

In some embodiments, the compositions comprising at least one AAV particle may be isolated or purified using the methods described in U.S. Pat. No. 8,524,446, the contents of which are herein incorporated by reference in its entirety.

### II. Formulations and Delivery

Pharmaceutical compositions and formulation

In addition to the pharmaceutical compositions (AAV particles comprising a modulatory polynucleotide sequence encoding the siRNA molecules), provided herein are pharmaceutical compositions which are suitable for administration to humans, it will be understood by the skilled artisan that such compositions are generally suitable for administration to any other animal, e.g, to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

In some embodiments, compositions are administered to subjects, humans, or human patients in need thereof. For the purposes of the present disclosure, the phrase “active ingredient” generally refers either to the synthetic siRNA duplexes, the modulatory polynucleotide encoding the siRNA duplex, or the AAV particle comprising a modulatory polynucleotide encoding the siRNA duplex described herein.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered.

The AAV particles comprising the modulatory polynucleotide sequence encoding the siRNA molecules of the present disclosure can be formulated using one or more excipients to: (1) increase stability; (2) increase cell transfection or transduction; (3) permit the sustained or delayed release; or (4) alter the biodistribution (e.g., target the AAV particle to specific tissues or cell types such as brain and neurons).

Formulations of the present disclosure can include, without limitation, saline, lipidoids, liposomes, lipid nanoparticles, polymers, lipoplexes, core-shell nanoparticles, peptides, proteins, cells transfected with AAV particles (e.g., for transplantation into a subject), nanoparticle mimics and combinations thereof. Further, the AAV particles of the present disclosure may be formulated using self-assembled nucleic acid nanoparticles.

Formulations of the pharmaceutical compositions described herein may be prepared by any method known or hereafter developed in the art of pharmacology. In general, such preparatory methods include the step of associating the active ingredient with an excipient and/or one or more other accessory ingredients.

A pharmaceutical composition in accordance with the present disclosure may be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may comprise between 0.1% and 99% w/w) of the active ingredient. By way of example, the composition may comprise between 0.1% and 100%, e.g., between and 50%, between 1-30%, between 5-80%, at least 80% (w/w) active ingredient.

In some embodiments, a pharmaceutically acceptable excipient may be at least 95%, at least 96%, at least 97% at least 98%, at least 99%, or 100% pure. In some embodiments, an excipient is approved for use for humans and for veterinary use. in some embodiments, an excipient may be approved by United States Food and Drug Administration. In some embodiments, an excipient may be of pharmaceutical grade. In some embodiments, an excipient may meet the standards of the United States Pharmacopoeia (USP). the European Pharmacopoeia (EP), the British Pharmacopoeia, and/or the International Pharmacopoeia.

Excipients, which, as used herein, includes, but is not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21^{st} Edition, A. R. Gennaro, Lippincott, Williams & Wilkins, Baltimore, Md., 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

in some embodiments, additional excipients that may be used in formulating the pharmaceutical composition may include MgCl₂, arginine, sorbitol, and/or trehalose.

In some embodiments, the formulations may comprise at least one inactive ingredient. As used herein, the term “inactive ingredient” refers to one or more inactive agents included in formulations. In some embodiments, all, none or some of the inactive ingredients which may be used in the formulations of the present disclosure may be approved by the US Food and Drug Administration (FDA).

Formulations of vectors comprising the nucleic acid sequence for the siRNA molecules of the present disclosure may include cations or anions. In some embodiments, the formulations include metal cations such as, but not limited to, Zn2+, Ca2+, Cu2+, Mg+ and combinations thereof.

As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418, Pharmaceutical Salts: Properties, Selection, and Use, P. H. Stahl and C. O. Wennuth (eds.), Wiley-VCH, 2008, and Berge et al., Journal of Pharmaceutical Science, 66, 1-19 (1977); the content of each of which is incorporated herein by reference in their entirety.

The term “pharmaceutically acceptable solvate,” as used herein, means a compound of the disclosure wherein molecules of a suitable solvent are incorporated in the crystal lattice. A suitable solvent is physiologically tolerable at the dosage administered. For example, solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformarnide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

According to the present disclosure, the AAV particle comprising the modulatory polynucleotide sequence encoding for the siRNA molecules may be formulated for CNS delivery. Agents that cross the brain blood barrier may be used. For example, some cell penetrating peptides that can target siRNA molecules to the brain blood barrier endothelium may be used to formulate the siRNA duplexes targeting the HTT gene.

### Sodium Phosphate

In some embodiments, at least one of the components in the formulation is sodium phosphate. The formulation may include monobasic, dibasic or a combination of both monobasic and dibasic sodium phosphate.

In some embodiments, the concentration of sodium phosphate in a formulation may be, but is not limited to, 0.1 mM, 0.2 mM, 0.3 mM, 0.4 mM, 0.5 mM, 0.6 mM, 0.7 mM, 0.8 mM, 0.9 mM, 1 mM, 1.1 mM, 1.2 mM, 1.3 mM, 1.4 mM, 1.5 mM, 1.6 mM, 1.7 mM, 1.8 mM, 1.9 mM, 2 mM, 2.1 mM, 2.2 mM, 2.3 mM, 2.4 mM, 2.5 mM, 2.6 mM, 2.7 mM, 2.8 mM, 2.9 mM, 3 mM, 3.1 mM, 3.2 mM, 3.3 mM, 3.4 mM, 3.5 mM, 3.6 mM, 3.7 mM, 3.8 3.9 mM, 4 mM, 4.1 mM, 4.2 mM, 4.3 mM, 4.4 mM, 4.5 mM, 4.6 mM, 4.7 mM, 4.8 mM, 4.9 mM, 5 M, 5.1 mM, 5.2 mM, 5.3 mM, 5.4 mM, 5.5 mM, 5.6 mM, 5.7 mM, 5.8 mM, 5.9 mM, 6 mM, 6.1 mM, 6.2 mM, 6.3 mM, 6.4 mM, 6.5 mM, 6.6 mM, 6.7 mM, 6.8 mM, 6.9 mM, 7 mM, 7.1 mM, 7.2 mM, 7.3 mM, 7.4 mM, 7.5 mM, 7.6 mM, 7.7 mM, 7.8 mM, 7.9 mM, 8 mM, 8.1 mM, 8.2 mM, 8.3 mM, 8.4 mM, 8.5 mM, 8.6 mM, 8.7 mM, 8.8 mM, 8.9 mM, 9 mM, 9.1 mM, 9.2 mM, 9.3 mM, 9.4 mM, 9.5 mM, 9.6 mM, 9.7 mM, 9.8 mM, 9.9 mM, 10 mM, 10.1 mM, 10.2 mM, 10.3 mM, 10.4 mM, 10.5 mM, 10.6 mM, 10.7 mM, 10.8 mM, 10.9 mM, 11 mM, 11.1 mM, 11.2 mM, 11.3 mM, 11.4 mM, 11.5 mM, 11.6 mM, 11.7 mM, 11.8 mM, 11.9 mM, 12 mM, 12.1 mM, 12.2 mM, 12.3 mM, 12.4 mM, 12.5 mM, 12.6 mM, 12.7 mM, 12.8 mM, 12.9 mM, 13 mM, 13.1 mM, 13.2 mM, 13.3 mM, 13.4 mM, 13.5 mM, 13.6 mM, 13.7 mM, 13.8 mM, 13.9 mM, 14 mM, 14.1 mM, 14.2 mM, 14.3 mM, 14.4 mM, 14.5 mM, 14.6 mM, 14.7 mM. 14.8 mM, 14.9 mM or 15 mM.

The formulation may include sodium phosphate in a range of 0-0.5 mM, 0.1-0.6 mM, 0.2-0.7 mM, 0.3-0.8 mM, 0.4-0.9 mM, 0.5-1 mM, 0.6-1.1 mM, 0.7-1.2 mM, 0.8-1.3 mM, 0.9-1.4 mM, 1-1.5 mM, 1.1-1.6 mM, 1.2-1.7 mM, 1.3-1.8 mM, 1.4-1.9 mM, 1.5-2 mM, 1.6-2.1 mM, 1.7-2.2 mM, 1.8-2.3 mM, 1.9-2.4 mM, 2-2.5 mM, 2.1-2.6 mM, 2.2-2.7 mM, 2.3-2.8 mM, 2.4-2.9 mM, 2.5-3 mM, 2.6-3.1 mM, 2.7-3.2 mM, 2.8-3.3 mM, 2.9-3.4 mM, 3-3.5 mM, 3.1-3.6 mM, 3.2-3.7 mM, 3.3-3.8 mM, 3.4-3.9 mM, 3.5-4 mM, 3.6-4.1 mM, 3.7-4.2 mM, 3.8-4.3 mM, 3.9-4.4 mM, 4-4.5 mM, 4.1-4.6 mM, 4.2-4.7 mM, 4.3-4.8 mM, 4.4-4.9 mM, 4.5-5 mM, 4.6-5.1 mM, 4.7-5.2 mM, 4.8-5.3 mM, 4.9-5.4 mM, 5-5.5 mM, 5.1-5.6 mM, 5.2-5.7 mM, 5.3-5.8 mM, 5.4-5.9 mM, 5.5-6 mM, 5.6-6.1 mM, 5.7-6.2 mM, 5.8-6.3 mM, 5.9-6.4 mM, 6-6.5 mM, 6.1-6.6 mM, 6.2-6.7 mM, 6.3-6.8 mM, 6.4-6.9 mM, 6.5-7 mM, 6.6-7.1 mM, 6.7-7.1 mM, 6.8-7.3 mM, 6.9-7.4 mM, 7-7.5 mM, 7.1-7.6 mM, 7.2-7.7 mM, 7.3-7.8 mM, 7.4-7.9 mM, 7.5-8 mM, 7.6-8.1 mM, 7.7-8.2 mM, 7.8-8.3 mM, 7.9-8.4 mM, 8-8.5 mM, 8.1-8.6 mM, 8.2-8.7 mM, 8.3-8.8 mM, 8.4-8.9 mM, 8.5-9 mM, 8.6-9.1 mM, 8.7-9.2 mM, 8.8-9.3 mM, 8.9-9.4 mM, 9-9.5 mM, 9.1-9.6 mM, 9.2-9.7 mM, 9.3-9.8 mM, 9.4-9.9 mM, 9.5-10 mM, 9.6-10.1 mM, 9.7-10.2 mM, 9.8-10.3 mM, 9.9-10.4 mM, 10-10.5 mM, 10.1-10.6 mM, 10.2-10.7 mM, 10.3-10.8 mM, 10.4-10.9 mM, 10.5-11 mM, 10.6-11.1 mM, 10.7-11.2 mM, 10.8-11.3 mM, 10.9-11.4 mM, 11-11.5 mM, 11.1-11.6 mM, 11.2-11.7 mM, 11.3-11.8 mM, 11.4-11.9 mM, 11.5-12 mM, 11.6-12.1 mM, 11.7-12.2 mM, 11.8-12.3 mM, 11.9-12.4 mM, 12-12.5 mM, 12.1-12.6 mM, 12.2-12.7 mM, 12.3-12.8 mM, 12.4-12.9 mM, 12.5-13 mM, 12.6-13.1 mM, 12.7-13.2 mM, 12.8-13.3 mM, 12.9-13.4 mM, 13-13.5 mM, 13.1-13.6 mM, 13.2-13.7 mM, 13.3-13.8 mM, 13.4-13.9 mM, 13.5-14 mM, 13.6-14.1 mM, 13.7-14.2 mM, 13.8-14.3 mM, 13.9-14.4 mM, 14-14.5 mM, 14.1-14.6 mM, 14.2-14.7 mM, 14.3-14.8 mM, 14.4-14.9 mM, 14.5-15 mM, 0-1 mM, 1-2 mM, 2-3 mM, 3-4 mM, 4-5 mM, 5-6 mM, 6-7 mM, 7-8 mM, 8-9 mM, 9-10 mM, 10-11 mM, 11-12 mM, 12-13 mM, 13-14 mM, 14-15 mM, 15-16 mM, 0-2 mM, 1-3 mM, 2-4 mM, 3-5 mM, 4-6 mM, 5-7 mM, 6-8 mM, 7-9 mM, 8-10 mM, 9-11 mM, 10-12 mM, 11-13 mM, 12-14 mM, 13-15 mM, 0-3 mM, 1-4 mM, 2-5 mM, 3-6 mM, 4-7 mM, 5-8 mM, 6-9 mM, 7-10 mM, 8-11 mM, 9-12 mM, 10-13 mM, 11-14 mM, 12-15 mM, 0-4 mM, 1-5 mM, 2-6 mM, 3-7 mM, 4-8 mM, 5-9 mM, 6-10 mM, 7-11 mM, 8-12 mM, 9-13 mM, 10-14 mM, 11-15 mM, 0-5 mM, 1-6 mM, 2-7 mM, 3-8 mM, 4-9 mM, 5-10 mM, 6-11 mM, 7-12 mM, 8-13 mM, 9-14 mM, 10-15 mM, 0-6 mM, 1-7 mM, 2-8 mM, 3-9 mM, 4-10 mM, 5-11 mM, 6-12 mM, 7-13 mM, 8-14 mM, 9-15 mM, 0-7 mM, 1-8 mM, 2-9 mM, 3-10 mM, 4-11 mM, 5-12 mM, 6-13 mM, 7-14 mM, 8-15 mM, 0-8 mM, 1-9 mM, 2-10 mM, 3-11 mM, 4-12 mM, 5-13 mM, 6-14 mM, 7-15 mM, 0-9 mM, 1-10 mM, 2-11 mM, 3-12 mM, 4-13 mM, 5-14 mM, 6-15 mM, 0-10 mM, 1-11 mM, 2-12 mM, 3-13 mM, 4-14 mM, 5-15 mM, 0-11 mM, 1-12 mM, 2-13 mM, 3-14 mM, 4-15 mM, 0-12 mM, 1-13 mM, 2-14 mM, 3-15 mM, 0-13 mM, 1-14 mM, 2-15 mM, 0-14 mM, 1-15 mM, or 0-15 mM.

In some embodiments, the formulation may include 0-10 mM of sodium phosphate.

In some embodiments, the formulation may include 2-3 mM of sodium phosphate.

In some embodiments, the formulation may include 2.7 mM of sodium phosphate.

In some embodiments, the formulation may include 9-10 mM of sodium phosphate.

In some embodiments, the formulation may include 10-11 mM of sodium phosphate.

In some embodiments, the formulation may include 10 mM of sodium phosphate.

### Potassium Phosphate

In some embodiments, at least one of the components in the formulation is potassium phosphate. The formulation may include monobasic, dibasic or a combination of both monobasic and dibasic potassium phosphate.

In some embodiments, the concentration of potassium phosphate in a formulation may be, but is not limited to, 0.1 mM, 0.2 mM, 0.3 mM, 0.4 mM, 0.5 mM, 0.6 mM, 0.7 mM, 0.8 mM, 0.9 mM, 1 mM, 1.1 mM, 1.2 mM, 1.3 mM, 1.4 mM, 1.5 mM, 1.6 mM, 1.7 mM, 1.8 mM, 1.9 mM, 2 mM, 2.1 mM, 2.2 mM, 2.3 mM, 2.4 mM, 2.5 mM, 2.6 mM, 2.7 mM, 2.8 mM, 2.9 mM, 3 mM, 3.1 mM, 3.2 mM, 3.3 mM, 3.4 mM, 3.5 mM, 3.6 mM, 3.7 mM, 3.8 mM, 3.9 mM, 4 mM, 4.1 mM, 4.2 mM, 4.3 mM, 4.4 mM, 4.5 mM, 4.6 mM, 4.7 mM, 4.8 mM, 4.9 mM, 5 mM, 5.1 mM, 5.2 mM, 5.3 mM, 5.4 mM, 5.5 mM, 5.6 mM, 5.7 mM, 5.8 mM, 5.9 mM, 6 mM, 6.1 mM, 6.2 mM, 6.3 mM, 6.4 mM, 6.5 mM, 6.6 mM, 6.7 mM, 6.8 mM, 6.9 mM, 7 mM, 7.1 mM, 7.2 mM, 7.3 mM, 7.4 mM, 7.5 mM, 7.6 mM, 7.7 mM, 7.8 mM, 7.9 mM, 8 mM, 8.1 mM, 8.2 mM, 8.3 mM, 8.4 mM, 8.5 mM, 8.6 mM, 8.7 mM, 8.8 mM, 8.9 mM, 9 mM, 9.1 mM, 9.2 mM, 9.3 mM, 9.4 mM, 9.5 mM, 9.6 mM, 9.7 M, 9.8 M, 9.9 M, 10 mM, 10.1 mM, 10.2 mM, 10.3 mM, 10.4 mM, 10.5 mM, 10.6 mM, 10.7 mM, 10.8 mM, 10.9 mM, 11 mM, 11.1 mM, 11.2 mM, 11.3 mM, 11.4 mM, 11.5 mM, 11.6 mM, 11.7 mM, 11.8 mM, 11.9 mM, 12 mM, 12.1 mM, 12.2 mM, 12.3 mM, 12.4 mM, 12.5 mM, 12.6 mM, 12.7 mM, 12.8 mM, 12.9 mM, 13 mM, 13.1 mM, 13.2 mM, 13.3 mM, 13.4 mM, 13.5 mM, 13.6 mM, 13.7 mM, 13.8 mM, 13.9 mM, 14 mM, 14.1 mM, 14.2 mM, 14.3 mM, 14.4 mM, 14.5 mM, 14.6 mM, 14.7 mM, 14.8 mM, 14.9 mM or 15 mM.

The formulation may include potassium phosphate in a range of 0-0.5 mM, 0.1-0.6 mM, 0.2-0.7 mM, 0.3-0.8 mM, 0.4-0.9 mM, 0.5-1 mM, 0.6-1.1 mM, 0.7-1.2 mM, 0.8-1.3 mM, 0.9-1.4 mM, 1-1.5 mM, 1.1-1.6 mM, 1.2-1.7 mM, 1.3-1.8 mM, 1.4-1.9 mM, 1.5-2 mM, 1.6-2.1 mM, 1.7-2.2 mM, 1.8-2.3 mM, 1.9-2.4 mM, 2-2.5 mM, 2.1-2.6 mM, 2.2-2.7 mM, 2.3-2.8 mM, 2.4-2.9 2.5-3 mM, 2.6-3.1 mM, 2.7-3.2 mM, 2.8-3.3 mM, 2.9-3.4 mM, 3-3.5 mM, 3.1-3.6 mM, 3.2-3.7 mM, 3.3-3.8 mM, 3.4-3.9 mM, 3.5-4 mM, 3.6-4.1 mM, 3.7-4.2 mM, 3.8-4.3 mM, 3.9-4.4 mM, 4-4.5 mM, 4.1-4.6 mM, 4.2-4.7 mM, 4.3-4.8 mM, 4.4-4.9 mM, 4.5-5 mM, 4.6-5.1 mM, 4.7-5.2 mM, 4.8-5.3 mM, 4.9-5.4 mM, 5-5.5 mM, 5.1-5.6 mM, 5.2-5.7 mM, 5.3-5.8 mM, 5.4-5.9 mM, 5.5-6 mM, 5.6-6.1 mM, 5.7-6.2 mM, 5.8-6.3 mM, 5.9-6.4 mM, 6-6.5 mM, 6.1-6.6 mM, 6.2-6.7 mM, 6.3-6.8 mM, 6.4-6.9 mM, 6.5-7 mM, 6.6-7.1 mM, 6.7-7.2 mM, 6.8-7.3 mM, 6.9-7.4 mM, 7-7.5 mM, 7.1-7.6 mM, 7.2-7.7 mM, 7.3-7.8 mM, 7.4-7.9 mM, 7.5-8 mM, 7.6-8.1 mM, 7.7-8.2 mM, 7.8-8.3 mM, 7.9-8.4 mM, 8-8.5 mM, 8.1-8.6 mM, 8.2-8.7 mM, 8.3-8.8 mM, 8.4-8.9 mM, 8.5-9 mM, 8.6-9.1 mM, 8.7-9.2 mM, 8.8-9.3 mM, 8.9-9.4 mM, 9-9.5 mM, 9.1-9.6 mM, 9.2-9.7 mM, 9.3-9.8 mM, 9.4-9.9 mM, 9.5-10 mM, 9.6-10.1 mM, 9.7-10.2 mM, 9.8-10.3 mM, 9.9-10.4 mM, 10-10.5 mM, 10.1-10.6 mM, 10.2-10.7 mM, 10.3-10.8 mM, 10.4-10.9 mM, 10.5-11 mM, 10.6-11.1 mM, 10.7-11.2 mM, 10.8-11.3 10.9-11.4 mM, 11-11.5 11.1-11.6 mM, 11.2-11.7 mM, 11.3-11.8 mM, 11.4-11.9 mM, 11.5-12 mM, 11.6-12.1 mM, 11.7-12.2 mM, 11.8-12.3 mM, 11.9-12.4 mM, 12-12.5 mM, 12.1-12.6 mM, 12.2-12.7 mM, 12.3-12.8 mM, 12.4-12.9 mM, 12.5-13 mM, 12.6-13.1 mM, 12.7-13.2 mM, 12.8-13.3 mM, 12.9-13.4 mM, 13-13.5 mM, 13.1-13.6 mM, 13.2-13.7 mM, 13.3-13.8 mM, 13.4-13.9 mM, 13.5-14 mM, 13.6-14.1 mM, 13.7-14.2 mM, 13.8-14.3 mM, 13.9-14.4 mM, 14-14.5 mM, 14.1-14.6 mM, 14.2-14.7 mM, 14.3-14.8 mM, 14.4-14.9 mM, 14.5-15 mM, 0-1 mM, 1-2 mM, 2-3 mM, 3-4 mM, 4-5 mM, 5-6 mM, 6-7 mM, 7-8 mM, 8-9 mM,9-10 mM, 10-11. mM, 11-12 mM, 12-13 mM, 13-14 mM,14-15 mM, 15-16 mM, 0-2 mM, 1-3 mM, 2-4 mM, 3-5 mM, 4-6 mM, 5-7 mM, 6-8 mM, 7-9 mM, 8-10 mM, 9-11 mM., 10-12 mM, 11-13 mM, 12-14 mM, 13-15 mM, 0-3 mM., 1-4 mM, 2-5 mM, 3-6 mM, 4-7 mM, 5-8 mM, 6-9 mM, 7-10 mM, 8-11 mM, 9-12 mM, 10-13 mM, 11-14 mM, 12-15 mM, 0-4 mM, 1-5 mM, 2-6 mM, 3-7 mM, 4-8 mM, 5-9 mM, 6-10 mM, 7-11 mM, 8-12 mM 9-13 mM, 10-14 mM, 11-15 mM, 0-5 mM, 1-6 mM, 2-7 mM, 3-8 mM, 4-9 mM, 5-10 mM, 6-11 mM, 7-12 mM, 8-13 mM, 9-14 mM, 10-15 mM, 0-6 mM, 1-7 mM, 2-8 mM, 3-9 mM, 4-10 mM, 5-11 mM, 6-12 mM, 7-13 mM, 8-14 mM, 9-15 mM, 0-7 mM, 1-8 mM, 2-9 mM, 3-10 mM, 4-11 mM, 5-12 mM, 6-13 mM, 7-14 mM, 8-15 mM, 0-8 mM, 1-9 mM, 2-10 mM, 3-11 mM, 4-12 mM, 5-13 mM, 6-14 mM, 7-15 mM, 0-9 mM, 1-10 mM, 2-11 mM, 3-12 mM, 4-13 mM, 5-14 mM, 6-15 mM, 0-10 mM, 1-11 mM, 2-12 mM, 3-13 mM, 4-14 mM, 5-15 mM, 0-11 mM, 1-12 mM, 2-13 mM, 3-14 mM, 4-15 mM, 0-12 mM, 1-13 mM, 2-14 mM, 3-15 mM, 0-13 mM, 1-14 mM, 2-15 mM, 0-14 mM, 1-15 mM, or 0-15 mM.

In some embodiments, the formulation may include 0-10 mM of potassium phosphate.

In some embodiments, the formulation may include 1-3 mM of potassium phosphate.

In some embodiments, the formulation may include 1-2 mM of potassium phosphate.

In some embodiments, the formulation may include 2-3 mM of potassium phosphate.

In some embodiments, the formulation may include 1.5 mM of potassium phosphate. In some embodiments, the formulation may include 1.54 mM of potassium phosphate.

In some embodiments, the formulation may include 2 mM of potassium phosphate.

### Sodium Chloride

In some embodiments, at least one of the components in the formulation is sodium chloride.

In some embodiments, the concentration of sodium chloride in a formulation may be, but is not limited to, 75 mM, 76 mM, 77 mM, 78 mM, 79 mM, 80 mM, 81 mM, 82 mM, 83 mM, 84 mM, 85 mM, 86 mM, 87 mM, 88 mM, 89 mM, 90 mM, 91 mM, 92 mM, 93 mM, 94 mM, 95 mM, 96 mM, 97 mM, 98 mM, 99 mM, 100 mM, 101 mM, 102 mM, 103 mM, 104 mM, 105 mM, 106 mM, 107 mM, 108 mM, 109 mM, 110 mM, 111 mM, 112 mM, 113 mM, 114 mM, 115 mM, 116 mM, 117 mM, 118 mM, 119 mM, 120 mM, 121 mM, 122 mM, 123 mM, 124 mM, 125 mM, 126 mM, 127 mM, 128 mM, 129 mM, 130 mM, 131 mM, 132 mM, 133 mM, 1.34 mM, 135 mM, 136 mM, 137 mM, 138 mM, 139 mM, 140 mM, 141 mM, 142 mM, 143 mM, 144 mM, 145 mM, 146 mM, 147 mM, 148 mM, 149 mM, 150 mM, 151 mM, 152 mM, 153 mM, 154 mM, 155 mM, 156 mM, 157 mM, 158 mM, 159 mM, 160 mM, 161 mM, 162 mM, 163 mM, 164 mM, 165 mM, 166 mM, 167 mM, 168 mM, 169 mM, 170 mM, 171 mM, 172 mM, 173 mM, 174 mM, 175 mM, 176 mM, 177 mM, 178 mM, 179 mM, 180 mM, 181 mM, 182 mM, 183 mM, 184 mM, 185 mM, 186 mM, 187 mM, 188 mM, 189 mM, 190 mM, 191 mM, 192 mM, 193 mM, 194 mM, 195 mM, 196 mM, 197 mM, 198 mM, 199 mM, 200 mM, 201 mM, 202 mM, 203 mM, 204 mM, 205 mM, 206 mM, 207 mM, 208 mM, 209 mM, 210 mM, 211 mM, 212 mM, 213 mM, 214 mM, 215 mM, 216 mM, 217 mM, 218 mM, 219 mM, or 220 mM.

The formulation may include sodium chloride in a range of 75-85 mM, 80-90 mM, 85-95 mM, 90-100 mM, 95-105 mM, 100-110 mM, 105-115 mM, 110-120 mM, 115-125 mM, 120-130 mM, 125-135 mM, 130-140 mM, 135-145 mM, 140-150 mM, 145-155 mM, 150-160 mM, 155-165 mM, 160-170 mM, 165-175 mM, 170-180 mM, 175-185 mM, 180-190 mM, 185-195 mM, 190-200 mM, 75-95 mM, 80-100 mM, 85-105 mM, 90-110 mM, 95-115 mM, 100-120 mM, 105-125 mM, 110-130 mM, 115-135 mM, 120-140 mM, 125-145 mM, 130-150 mM, 135-155 mM, 140-160 mM, 145-165 mM, 150-170 mM, 155-175 mM, 160-180 mM, 165-185 mM, 170-190 mM, 175-195 mM, 180-200 mM, 75-100 mM, 80-105 mM, 85-110 mM, 90-115 mM, 95-120 mM, 100-125 mM, 105-130 mM, 110-135 mM, 115-140 mM, 120-145 mM, 125-150 mM, 130-155 mM, 135-160 mM, 140-165 mM, 145-170 mM, 150-175 mM, 155-180 mM, 160-185 mM, 165-190 mM, 170-195 mM, 175-200 mM, 75405 mM, 80-110 mM, 85-115 mM, 90-120 mM, 95-125 mM, 100-130 mM, 105-135 mM, 110-140 mM, 115-145 mM, 120-150 mM, 125-155 mM, 130-160 mM, 135-165 mM, 140-170 mM, 145-175 mM, 150-180 mM, 155-185 mM, 160-190 mM, 165-195 mM, 170-200 mM, 75-115 mM, 80-120 mM, 85-125 mM, 90-130 mM, 95-135 mM, 100-140 mM, 105-145 mM, 110-150 mM, 115-155 mM, 120-160 mM, 125-165 mM, 130-170 mM, 135-175 mM, 140-180 mM, 145-185 mM, 150-190 mM, 155-195 mM, 160-200 mM, 75-120 mM, 80-125 mM, 85-130 mM, 90-135 mM, 95-140 mM, 100-145 mM, 105-150 mM, 110-155 mM, 115-160 mM, 120-165 mM, 125-170 mM, 130-175 mM, 135-180 mM, 140-185 mM, 145-190 mM, 150-195 mM, 155-200 mM, 75-125 mM, 80-130 mM, 85-135 mM, 90-140 mM, 95-145 mM, 100-150 mM, 105-155 mM, 110-160 mM, 115-165 mM, 120-170 mM, 125-175 mM, 130-180 mM, 135-185 mM, 140-190 mM, 145-195 mM, 150-200 mM, 75-125 mM, 80-130 mM, 85-135 mM, 90-140 mM, 95-145 mM, 100-150 mM, 105-155 mM, 110-160 mM, 115-165 mM, 120-170 mM, 125-175 mM, 130-180 mM, 135-185 mM, 140-190 mM, 145-195 mM, 150-200 mM, 75-135 mM, 80-140 mM, 85-145 mM, 90-150 mM, 95-155 mM, 100-160 mM, 105-165 mM, 110-170 mM, 115-175 mM, 120-180 mM, 125-185 mM, 130-190 mM, 135-195 mM, 140-200 mM, 75-145 mM, 80-150 mM, 85-155 mM, 90-160 mM, 95-165 mM, 100-170 mM, 105-175 mM, 110-180 mM, 115-185 mM, 120-190 mM, 125-195 mM, 130-200 mM, 75-155 mM, 80-160 mM, 85-165 mM, 90-170 mM, 95-175 mM, 100-180 mM, 105-185 mM, 110-190 mM, 115-195 mM, 120-200 mM, 75-165 mM, 80-170 mM, 85-175 mM, 90-180 mM, 95-185 mM, 100-190 mM, 105-195 mM, 110-200 mM, 75-175 mM, 80-180 mM, 85-185 mM, 90-190 mM, 95-195 mM, 100-200 mM, 80-220 mM, 90-220 mM, 100-220 mM, 110-220 mM, 120-220 mM, 130-220 mM, 140-220 mM, 150-220 mM, 160-220 mM, 170-220 mM, 180-220 mM, 190-220 mM, 200-220 mM, or 210-220 mM.

In some embodiments, the formulation may include 80-220 mM of sodium chloride.

In some embodiments, the formulation may include 80-150 mM of sodium chloride.

In some embodiments, the formulation may include 75 mM of sodium chloride.

in some embodiments, the formulation may include 83 mM of sodium chloride.

In some embodiments, the formulation may include 92 mM of sodium chloride.

In some embodiments, the formulation may include 95 mM of sodium chloride.

In some embodiments, the formulation may include 98 mM of sodium chloride

In some embodiments, the formulation may include 100 mM, of sodium chloride.

In some embodiments, the formulation may include 107 mM of sodium chloride.

In some embodiments, the formulation may include 109 mM of sodium chloride.

In some embodiments, the formulation may include 118 mM of sodium chloride.

In some embodiments, the formulation may include 125 mM of sodium chloride.

In some embodiments, the formulation may include 127 m41 of sodium chloride.

In some embodiments, the formulation may include 133 mM of sodium chloride.

In some embodiments, the formulation may include 142 in4i of sodium chloride.

In some embodiments, the formulation may include 150 moi of sodium chloride

In some embodiments, the formulation may include 155 mM of sodium chloride.

In some embodiments, the formulation may include 192 mM of sodium chloride.

In some embodiments, the formulation may include 210 mM of sodium chloride.

Potassium chloride

In some embodiments, at least one of the components in the formulation is potassium chloride.

In some embodiments, the concentration of potassium chloride in a formulation may be, but is not limited to, 0.1 mM, 0.2 mM, 0.3 mM, 0.4 mM, 0.5 mM, 0.6 mM, 0.7 mM, 0.8 mM, 0.9 mM, 1 mM, 1.1 mM, 1.2 mM, 1.3 mM, 1.4 mM, 1.5 mM, 1.6 mM, 1.7 mM, 1.8 mM, 1.9 mM, 2 mM, 2.1 mM, 2.2 mM, 2.3 mM, 2.4 mM, 2.5 mM, 2.6 mM, 2.7 mM, 2.8 mM, 2.9 mM, 3 mM, 3.1 mM, 3.2 mM, 3.3 mM, 3.4 mM, 3.5 mM, 3.6 mM, 3.7 mM, 3.8 mM, 3.9 mM, 4 mM, 4.1 mM, 4.2 mM, 4.3 mM, 4.4 mM, 4.5 mM, 4.6 mM, 4.7 mM, 4.8 mM, 4.9 mM, 5 mM, 5.1 mM, 5.2 mM, 5.3 mM, 5.4 mM, 5.5 mM, 5.6 M, 5.7 mM, 5.8 mM, 5.9 mM, 6 mM, 6.1 mM, 6.2 mM, 6.3 mM, 6.4 mM, 6.5 mM, 6.6 mM, 6.7 mM, 6.8 mM, 6.9 mM, 7 mM, 7.1 mM, 7.2 mM, 7.3 mM, 7.4 mM, 7.5 mM, 7.6 mM, 7.7 mM, 7.8 mM, 7.9 mM, 8 mM, 8.1 mM, 8.2 mM, 8.3 mM, 8.4 mM, 8.5 mM, 8.6 mM, 8.7 mM, 8.8 mM, 8.9 mM, 9 mM, 9.1 mM, 9.2 mM, 9.3 mM, 9.4 mM, 9.5 mM, 9.6 mM, 9.7 mM, 9.8 mM, 9.9 mM, 10 mM, 10.1 mM, 10.2 mM, 10.3 mM, 10.4 mM, 10.5 mM, 10.6 mM, 10.7 mM, 10.8 mM, 10.9 mM, 11 mM, 11.1 mM, 11.2 mM, 11.3 mM, 11.4 mM, 11.5 mM, 11.6 mM, 11.7 mM, 11.8 mM, 11.9 mM, 12 mM, 12.1 mM, 12.2 mM, 12.3 mM, 12.4 mM, 12.5 mM, 12.6 mM, 12.7 mM, 12.8 mM, 12.9 mM, 13 mM, 13.1 mM, 13.2 mM, 13.3 mM, 13.4 mM, 13.5 mM, 13.6 mM, 13.7 mM, 13.8 mM, 13.9 mM, 14 mM, 14.1 mM, 14.2 mM, 14.3 mM, 14.4 mM, 14.5 mM, 14.6 mM, 14.7 mM, 14.8 mM, 14.9 mM or 15 mM.

The formulation may include potassium chloride in a range of 0-0.5 mM, 0.1-0.6 mM, 0.2-0.7 mM, 0.3-0.8 mM, 0.4-0.9 mM, 0.5-1 mM, 0.6-1.1 mM, 0.7-1.2 mM, 0.8-1.3 mM, 0.9-1.4 mM, 1-1.5 mM, 1.1-1.6 mM, 1.2-1.7 mM, 1.3-1.8 mM, 1.4-1.9 mM, 1.5-2 mM, 1.6-2.1 mM, 1.7-2.2 mM, 1.8-2.3 mM, 1.9-2.4 mM, 2-2.5 mM, 2.1-2.6 mM, 2.2-2.7 mM, 2.3-2.8 mM, 2.4-2.9 mM, 2.5-3 mM, 2.6-3.1 mM, 2.7-3.2 mM, 2.8-3.3 mM, 2.9-3.4 mM, 3-3.5 mM, 3.1-3.6 mM, 3.2-3.7 mM, 3.3-3.8 mM, 3.4-3.9 mM, 3.5-4 mM, 3.6-4.1 mM, 3.7-4.2 mM, 3.8-4.3 mM, 3.9-4.4 mM, 4-4.5 mM, 4.1-4.6 mM, 4.2-4.7 mM, 4.3-4.8 mM, 4.4-4.9 mM, 4.5-5 mM, 4.6-5.1 mM, 4.7-5.2 mM, 4.8-5.3 mM, 4.9-5.4 mM, 5-5.5 mM, 5.1-5.6 mM, 5.2-5.7 mM, 5.3-5.8 mM, 5.4-5.9 mM, 5.5-6 mM, 5.6-6.1 mM, 5.7-6.2 mM, 5.8-6.3 mM, 5.9-6.4 mM, 6-6.5 mM, 6.1-6.6 mM, 6.2-6.7 mM, 6.3-6.8 mM, 6.4-6.9 mM, 6.5-7 mM, 6.6-7.1 mM, 6.7-7.2 mM, 6.8-7.3 mM, 6.9-7.4 mM, 7-7.5 mM, 7.1-7.6 mM, 7.2-7.7 mM, 7.3-7.8 mM, 7.4-7.9 mM, 7.5-8 mM, 7.6-8.1 mM, 7.7-8.2 mM, 7.8-8.3 mM, 7.9-8.4 mM, 8-8.5 mM, 8.1-8.6 mM, 8.2-8.7 mM, 8.3-8.8 mM, 8.4-8.9 mM, 8.5-9 mM, 8.6-9.1 mM, 8.7-9.2 mM, 8.8-9.3 mM, 8.9-9.4 mM, 9-9.5 mM, 9.1-9.6 mM, 9.2-9.7 mM, 9.3-9.8 mM, 9.4-9.9 mM, 9.5-10 mM, 9.6-10.1 mM, 9.7-10.2 mM, 9.8-10.3 mM, 9.9-10.4 mM, 10-10.5 mM, 10.1-10.6 mM, 10.2-10.7 mM, 10.3-10.8 mM, 10.4-10.9 mM, 10.5-11 mM, 10.6-11.1 mM, 10.7-11.2 mM, 10.8-11.3 mM, 10.9-11.4 mM, 11-11.5 mM, 11.1-11.6 mM, 11.2-11.7 mM, 11.3-11.8 mM, 11.4-11.9 mM, 11.5-12 mM, 11.6-12.1 mM, 11.7-12.2 mM, 11.8-12.3 mM, 11.9-12.4 mM, 12-12.5 mM, 12.1-12.6 mM, 12.2-12.7 mM, 12.3-12.8 mM, 12.4-12.9 mM, 12.5-13 mM, 12.6-13.1 mM, 12.7-13.2 mM, 12.8-13.3 mM, 12.9-13.4 mM, 13-13.5 mM, 13.1-13.6 mM, 13.2-13.7 mM, 13.3-13.8 mM, 13.4-13.9 mM, 13.5-14 mM, 13.6-14.1 mM, 13.7-14.2 mM, 13.8-14.3 mM, 13.9-14.4 mM, 14-14.5 mM, 14.1-14.6 mM, 14.2-14.7 mM, 14.3-14.8 mM, 14.4-14.9 mM, 14.5-15 mM, 0-1 mM, 1-2 mM, 2-3 mM, 3-4 mM, 4-5 mM, 5-6 mM, 6-7 mM, 7-8 mM, 8-9 mM, 9-10 mM, 10-11 mM, 11-12 mM, 12-13 mM, 13-14 mM, 14-15 mM, 15-16 mM, 0-2 mM, 1-3 mM, 2-4 mM, 3-5 mM, 4-6 mM, 5-7 mM, 6-8 mM, 7-9 mM, 8-10 mM, 9-11 mM, 10-12 mM, 11-13 mM, 12-14 mM, 13-15 mM, 0-3 mM, 1-4 mM, 2-5 mM, 3-6 mM, 4-7 mM, 5-8 mM, 6-9 mM, 7-10 mM, 8-11 mM, 9-12 mM, 10-13 mM, 11-14 mM, 12-15 mM, 0-4 mM, 1-5 mM, 2-6 mM, 3-7 mM, 4-8 mM, 5-9 mM, 6-10 mM, 7-11 mM, 8-12 mM, 9-13 mM, 10-14 mM, 11-15 mM, 0-5 mM, 1-6 mM, 2-7 mM, 3-8 mM, 4-9 mM, 5-10 mM, 6-11 mM, 7-12 mM, 8-13 mM, 9-14 mM, 10-15 mM, 0-6 mM, 1-7 mM, 2-8 mM, 3-9 mM, 4-10 mM, 5-11 mM, 6-12 mM, 7-13 mM, 8-14 mM, 9-15 mM, 0-7 mM, 1-8 mM, 2-9 mM, 3-10 mM, 4-11 mM, 5-12 mM, 6-13 mM, 7-14 mM, 8-15 mM, 0-8 mM, 1-9 mM, 2-10 mM, 3-11 mM, 4-12 mM, 5-13 mM, 6-14 mM, 7-15 mM, 0-9 mM, 1-10 mM, 2-11 mM, 3-12 mM, 4-13 mM, 5-14 mM, 6-15 mM, 0-10 mM, 1-11 mM, 2-12 mM, 3-13 mM, 4-14 mM, 5-15 mM, 0-11 mM, 1-12 mM, 2-13 mM, 3-14 mM, 4-15 mM, 0-12 mM, 1-13 mM, 2-14 mM, 3-15 mM, 0-13 mM, 1-14 mM, 2-15 mM, 0-14 mM, 1-15 mM, or 0-15 mM.

In some embodiments, the formulation may include 0-10 mM of potassium chloride.

In some embodiments, the formulation may include 1-3 ml-f of potassium chloride.

In some embodiments, the formulation may include 1-2 mM of potassium chloride.

In some embodiments, the formulation may include 2-3 mM of potassium chloride.

In some embodiments, the formulation may include 1.5 mM of potassium chloride.

In some embodiments, the formulation may include 2.7 moi of potassium chloride.

### Magnesium Chloride

In some embodiments, at least one of the components in the formulation is magnesium chloride.

In some embodiments, the concentration of magnesium chloride may be, but is not limited to, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 1.9 mM, 20 mM, 21 mM, 22 mM, 23 mM, 24 mM, 25 mM, 26 mM, 27 mM, 28 mM, 29 mM, 30 mM, 31 mM, 32 mM, 33 mM, 34 mM, 35 mM, 36 mM, 37 mM, 38 mM, 39 mM, 40 mM, 41 mM, 42 mM, 43 mM, 44 mM, 45 mM, 46 mM, 47 mM, 48 mM, 49 mM, 50 mM, 51 mM, 52 mM, 53 mM, 54 mM, 55 mM, 56 mM, 57 mM, 58 mM, 59 mM, 60 mM, 61 mM, 62 mM, 63 mM, 64 mM, 65 mM, 66 mM, 67 mM, 68 mM, 69 mM, 70 mM, 71 mM, 72 mM, 73 mM, 74 mM, 75 mM, 76 mM, 77 mM, 78 mM, 79 mM, 80 mM, 81 mM, 82 mM, 83 mM, 84 mM, 85 mM, 86 mM, 87 mM, 88 mM, 89 mM, 90 mM, 91 mM, 92 mM, 93 mM, 94 mM, 95 mM, 96 mM, 97 mM, 98 mM, 99 mM, or 100 mM.

The formulation may include magnesium chloride in a range of 0-5 mM, 1-5 mM, 2-5 mM, 3-5 mM, 4-5 mM, 0-10 mM, 1-10 mM, 2-10 mM, 3-10 mM, 4-10 mM, 5-10 mM, 6-10 mM, 7-10 mM, 8-10 mM, 9-10 mM, 0-25 mM, 1-25 mM, 2-25 mM, 3-25 mM, 4-25 mM, 5-25 mM, 6-25 mM,7-25 mM, 8-25 mM, 9-25 mM, 10-25 mM,11-25 mM, 12-25 mM, 13-25 mM, 14-25 mM, 15-25 mM, 16-25 mM, 17-25 mM, 18-25 mM, 19-25 mM, 20-25 mM, 21-25 mM, 22-25 mM, 23-25 mM, 24-25 mM, 0-50 mM, 1-50 mM, 2-50 mM, 3-50 mM, 4-50 mM, 5-50 mM, 6-50 mM, 7-50 mM, 8-50 mM, 9-50 mM, 10-50 mM, 11-50 mM, 12-50 mM, 13-50 mM, 14-50 mM,15-50 mM, 16-50 mM, 17-50 mM, 18-50 mM, 19-50 mM, 20-50 mM, 21-50 mM, 22-50 mM, 23-50 mM, 24-50 mM, 25-50 mM, 26-50 mM, 27-50 mM, 28-50 mM, 29-50 mM, 30-50 mM, 31-50 mM, 32-50 mM, 33-50 mM, 34-50 mM, 35-50 mM, 36-50 mM, 37-50 mM, 38-50 mM, 39-50 mM, 40-50 mM, 41-50 mM, 42-50 mM, 43-50 mM, 44-50 mM, 45-50 mM, 46-50 mM, 47-50 mM, 48-50 mM, 49-50 mM, 0-75 mM, 1-75 mM, 2-75 mM, 3-75 mM, 4-75 mM, 5-75 mM, 6-75 mM, 7-75 mM, 8-75 mM, 9-75 mM, 10-75 mM, 11-75 mM, 12-75 mM, 13-75 mM, 14-75 mM, 15-75 mM, 16-75 mM, 17-75 mM, 18-75 mM, 19-75 mM, 20-75 mM, 21-75 mM, 22-75 mM, 23-75 mM, 24-75 mM, 25-75 mM, 26-75 mM, 27-75 mM, 28-75 mM, 29-75 mM, 30-75 mM, 31-75 mM, 32-75 mM, 33-75 mM, 34-75 mM, 35-75 mM, 36-75 mM, 37-75 mM, 38-75 mM, 39-75 mM, 40-75 mM, 41-75 mM, 42-75 mM, 43-75 mM, 44-75 mM, 45-75 mM, 46-75 mM, 47-75 mM, 48-75 mM, 49-75 mM, 50-75 mM, 51-75 mM, 52-75 mM, 53-75 mM, 54-75 mM, 55-75 mM, 56-75 mM, 57-75 mM, 58-75 mM, 59-75 mM, 60-75 mM, 61-75 mM, 62-75 mM, 63-75 mM, 64-75 mM, 65-75 mM, 66-75 mM, 67-75 mM, 68-75 mM, 69-75 mM, 70-75 mM, 71-75 mM, 72-75 mM, 73-75 mM, 74-75 mM, 50-100 mM, 60-100 mM, 75-100 mM, 80-100 mM, or 90-100 mM.

### Sugar

In some embodiments, the formulation may include at least one sugar and/or sugar substitute.

In some embodiments, the formulation may include at least one sugar and/or sugar substitute to increase the stability of the formulation. This increase in stability may provide longer hold times for in-process pools, provide a longer “shelf-life”, increase the concentration of AAV particles in solution (e.g., the formulation is able to have higher concentrations of AAV particles without rAAV dropping out of the solution) and/or reduce the generation or formation of aggregation in the formulations.

In some embodiments, the inclusion of at least one sugar and/or sugar substitute in the formulation may increase the stability of the formulation by 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or more than 95%, 1-5%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20- 55%, 20-60%, 20-65%, 20-70%, 70-75%, 70-80%, 70-85%, 70-90%, 20-95%, 25-35%, 25-40%, 25-45%, 25-50%, 25-55%, 25-60%, 25-65%, 25-70%, 25-75%, 25-80%, 25-85%, 25-90%, 25- 95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95% as compared to the same formulation without the sugar and/or sugar substitute.

In some embodiments, the sugar and/or sugar substitute is used in combination with a phosphate buffer for increased stability. The combination of the sugar and/or sugar substitute with the phosphate butter may increase stability by 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or more than 95%, 1-5%, 5-15%, 5-20%, 5-25%, 5-30%, 5-35%, 5-40%, 5-45%, 5-50%, -55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95° 0.25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 15-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95% as compared to the same formulation without the sugar and/or sugar substitute. As a non-limiting example, the sugar is sucrose.

In some embodiments, formulations of pharmaceutical compositions described herein may comprise a disaccharide. Suitable disaccharides that may be used in the formulation described herein may include sucrose, lactulose, lactose, maltose, trehalose, cellobiose, chitobiose, kojibiose, nigerose, isomaltose. β,β-trehalose, α,β-trehalose, sophorose, laminaribiose, gentiobiose, turanose, maltulose, palatinose, gentiobiulose, mannobiose, melibiose, melibiulose, rutinose, rutinulose, and xylobiose. The concentration of disaccharide (w/v) used in the formulation may be between 1%-15%, for example, between 1%-5%, between 3%-6%, between 5%-8%, between 7%-10%, or between 10%-15%,

In some embodiments, the formulation may include at least one disaccharide which is sucrose.

In some embodiments, the formulation may include sucrose at 0.1%, 0.2%, 0.3%, 0.4%, 0.5%, 0.6%, 0.7%, 0.8%, 0.9%, 1%, 1.1%, 1.2%, 1.3%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5%, 5.1%, 5.2%, 5.3%, 5.4%, 5.5%, 5.6%, 5.7%, 5.8%, 5.9%, 6%, 6.1%, 6,2%, 6.3%, 6.4%, 6.6%, 6.7%, 6.8%, 6.9%, 7%, 7.1%, 7.2%, 7.3%, 7.4%, 7.5%, 7.6%, 7.7%, 7.8%, 7.9%, 8%, 8.1%, 8.2%, 8.3%, 8.4%, 8.5%, 8.6%, 8.7%, 8.8%, 8.9%, 9%, 9.1%, 9.2%, 9.3%, 9.4%, 9.5%, 9.6%, 9.7%, 9.8%, 9.9%, or 10% w/v.

In some embodiments, the formulation may include sucrose in a range of 0-1%, 0.1-1%, 0.2-1%, 0.3-1%, 0.4-1%, 0.5-1%, 0.6-1%, 0.7-1%, 0.8-1%, 0.9-1%, 0-1.5%, 0.1-1.5%, 0.2-1.5%, 0.3-1.5%, 0.4-1.5%, 0.5-1.5%, 0.6-1.5%, 0.7-1.5%, 0.8-1.5%, 0.9-1.5%, 1-1.5%, 1.1-1.5%, 1.2-1.5%, 1.3-1.5%, 1.4-1.5%, 0-2%, 0.1-2%, 0.2-2%, 0.3-2%, 0.4-2%, 0.5-2%, 0.6-2%, 0.7-2%, 0.8-2%, 0.9-2%, 1-2%, 1.1-2%, 1.2-2%, 1.3-2%, 1.4-2%, 1.5-2%, 1.6-2%, 1.7-2%, 1.8-2%, 1.9-2%, 0-2.5%, 0.1-2.5%, 0.2-2.5%, 0.3-2.5%, 0.4-2.5%, 0.5-2.5%, 0.6-2.5%, 0.7-2.5%, 0.8-2.5%, 0.9-2.5%, 1-2.5%, 1.1-2.5%, 1.2-2.5%, 1.3-2.5%, 1.4-2.5%, 1.5-2.5%, 1.6-2.5%, 1.7-2.5%, 1.8-2.5%, 1.9-2.5%, 2-2.5%, 2.1-2.5%, 2.2-2.5%, 2.3-2.5%, 2.4-2.5%, 0-3%, 0.1-3%, 0.2-3%, 0.3-3%, 0.4-3 %, 0.5-3%, 0.6-3 %, 0.7-3%, 0.8-3%, 0.9-3%, 1-3%, 1.1-3%, 1.2-3%, 1.3-3%, 1.4-3%, 1.5-3%, 1.6-3%, 1.7-3%, 1.8-3%, 1.9-3%, 2-3%, 2.1-3%, 2.2-3%, 2.3-3%, 2.4-3%, 2.5-3%, 2.6-3%, 2.7-3%, 2.8-3%, 2.9-3%, 0-3.5%, 0.1-3.5%, 0.2-3.5%, 0.3-3.5%, 0.4-3.5%, 0.5 -3.5 %, 0.6-3.5%, 0.7-3.5%, 0.8-3.5%, 0.9-3.5%, 1-3.5%, 1. 1-3.5%, 1.2-3.5%, 1.3-3.5%, 1.4-3.5 %, 1.6-3.5%, 1.7-3.5%, 1.8-3.5 %, 1.9-3.5%, 2-3.5%, 2.1-3.5%, 2.2-3.5%, 2.3 -3.5%, 2.4-3.5%, 2.5-3.5%, 2.6-3.5%, 2.7-3.5%, 2.8-3.5%, 2.9-3.5%, 3-3.5%, 3.1-3.5%, 3.2-3.5%, 3.3-3.5%, 3.4-3.5%, 0-4%, 0.1-4%, 0.2-4%, 0.3-4%, 0.4-4%, 0.5-4%, 0.6-4%, 0.7-4%, 0.8-4%, 0.9-4%, 1-4%, 1.1-4%, 1.2-4%, 1.3-4%, 1.4-4%, 1.5-4%, 1.6-4%, 1.7-4%, 1.8-4%, 1.9-4%, 2-4%, 2.1-4%, 2.2-4%, 2.3-4%, 2.4-4%, 2.5-4%, 2.6-4%, 2.7-4%, 2.8-4%, 2.9-4%, 3-4%, 3.1-4%, 3.2-4%, 3.34%, 3.4-4%, 3.5-4%, 3.64%, 3.7-4%, 3.84%, 3.9-4%, 0-4.5), 0.1-4.5%, 0.2-4.5%, 0.3-4.5%, 0.4-4.5%, 0.5-4.5%, 0.6-4.5%, 0.7-4.5%, 0.8-4.5%, 0.9-4.5%, 1-4.5%, 1.1-4.5%, 1.2-4.5%, 1.3-45%, 1.4-4.5%, 1.5-4.5%, 1.6-4.5%, 1.7-4.5%, 1.8-4.5%, 1.9-4.5%, 2-4.5%, 2.1-4.5%, 2.2-4.5%, 2.3-4.5%, 2.4-4.5%, 2.5-4.5%, 2.6-4.5%, 2.7-4.5%, 2.8-4.5%, 2.9-4.5%, 3-4.5%, 3.1-4.5%, 3.2-4.5%, 3.3-4.5%, 3.4-4.5%, 3.5-4.5%, 3.6-4.5%, 3.7-4.5%, 3.8-4.5%, 3.9-4.5%, 4-4.5%, 4.1-4.5%, 4.2-4.5%, 4.3-4.5%, 4.4-4.5%, 0-5%, 0.1-5%, 0.2-5%, 0.3-5 %, 0.4-5%, 0.5-5 %, 0.6-5%, 0.7-5 %, 0.8-5%, 0.9-5%, 1-5%, 1.1-5%, 1.2-5%, 1.3-5%, 1.4-5%, 1.5-5%, 1.6-5%, 1.7-5%, 1.8-5%, 1.9-5%, 2-5%, 2.1-5%, 2.2-5%, 2.3-5%, 2.4-5%, 2.5-5%, 2.6-5%, 2.7-5%, 2.8-5%, 2.9-5%, 3-5%, 3.1-5%, 3.2-5%, 3.3-5%, 3.4-5%, 3.5-5%, 3.6-5%, 3.7-5%, 3.8-5%, 3.9-5%, 4-5%, 4.1-5%, 4.2-5%, 4.3-5%, 4.4-5%, 4.5-5%, 4.6-5%, 4.7-5%, 4.8-5%, 4.9-5%, 0-5.5%, 0.1-5.5%, 0.2-5.5%, 0.3-5.5%, 0.4-5.5%, 0.5-5.5%, 0.6-5.5%, 0.7-5.5%, 0.8-5.5%, 0.9-5.5%, 1-5.5%, 1.1-5.5%, 1.2-5.5%, 1.3-5.5%, 1.4-5.5%, 1.5-5.5%, 1.6-5.5%, 1.7-5.5%, 1.8-5.5 %, 1.9-5.5%, 2-5.5%, 2.1-5.5%, 2.2-5.5%, 2.3-5.5%, 2.4-5.5%, 2.5-5.5%, 2.6-5.5%, 2.7-5.5%, 2.8-5.5%, 2.9-5.5%, 3-5.5%, 3.1-5.5%, 3.2-5.5%, 3.3-5.5%, 3.4-5.5%, 3.5-5.5%, 3.6-5.5%, 5.5%, 3.8-5.5%, 3.9-5.5%, 4-5.5%, 4.1-5.5%, 4.2-5.5%, 4.3-5.5%, 4.4-5.5%, 4.5-5.5%, 4.6-5.5%, 4.7-5.5%, 4.8-5.5%, 4.9-5.5%, 5-5.5%, 5.1-5.5%, 5.2-5.5%, 5.3-5.5%, 5.4-5.5%, 0-6%, 0.1-6%, 0.2-6%, 0.3-6%, 0.4-6%, 0.5-6%, 0.6-6%, 0.7-6%, 0,8-6%, 0.9-6%, 1-6%, 1.1-6%, 1.2-6%, 1.3-6%, 1.4-6%, 1.5-6%, 1.6-6%, 1.7-6%, 1.8-6%, 1.9-6%, 2-6%, 2.1-6%, 2.2-6%, 2.3-6%, 2.4-6%, 2.5-6%, 2.6-6%, 2.7-6%, 28-6%, 2.9-6%, 3-6%, 3.1-6%, 3.2-6%, 3.3-6%, 3.4-6%, 3.5-6%, 3.6-6%, 3.7-6%, 3.8-6%, 3.9-6%, 4-6%, 4.1-6%, 4.2-6%, 4.3-6%, 4.4-6%, 4.5- 6%, 4.6-6%, 4.7-6%), 4.8-6%, 4.9-6%, 5-6%, 5.1-6%, 5.2-6%, 5.3-6%, 5.4-6%, 5.5-6%, 5.6-6%, 5.7-6%, 5.8-6%, 5.9-6%, 0-6.5%, 0.1-6.5%, 0.2-6.5%, 0.3-6.5%, 0.4-6.5%, 0.5-6.5%, 0.6-6.5%, 0.7-6.5%, 0.8-6.5%, 0.9-6.5%, 1-6.5%, 1.1-6.5%, 1.2-6.5%, 1.3-6.5%, 1.4-6.5%, 1.5-6.5%, 1.6-6.5%, 1.7-6.5%, 1.8-6.5%, 1.9-6.5%, 7-6.5%, 2.1-6.5%, 2.2-6.5%, 2.3-6.5%, 7.4-6.5%, 2.5 -6.5%, 2.6-6.5%, 2.7-6.5%, 2.8-6.5%, 2.9-6.5° A, 3-6.5%, 3.1-6.5%, 3.2-6.5%, 3.3-6.5%, 3.4-6.5%, 3.5-6.5%, 3.6-6.5%, 3.7-6.5%, 3.8-6.5%, 3.9-6.5%, 4-6.5%, 4.1-6.5%, 4.2-6.5%, 4.3-6.5%, 4.4-6.5%, 4.5-6.5%, 4.6-6.5%, 4.7-6.5%, 4.8-6.5%, 4.9-6.5%, 5-6.5%, 5.1-6.5%, 5.2-6.5%, 5.3-6.5%, 5.4-6.5%, 5.5-6.5%, 5.6-6.5%, 5.7-6.5%, 5.8-6.5%, 5.9-6.5%, 6-6.5%, 6.1-6.5%, 6.2-6.5%, 6.3-6.5%, 6.4-6.5%, 0-7%, 0.1-7%, 0.2-7%, 0.3-7%, 0.4-7%, 0.5-7%, 0.6-7%, 0.7-7%, 0.8-7%, 0.9-7%, 1-7%, 1.1-7%, 1.2-7%, 1.3-7%, 1.4-7%, 1.5-7%, 1.6-7%, 1.7-7%, 1.8-7%, 1.9-7%, 2-7%, 2.1-7%, 2.2-7%, 2.3-7%, 2.4-7%, 2.5-7%, 2.6-7%, 2.7-7%, 2.8-7%, 2.9-7%, 3-7%, 3.1-7%, 3.2-7%, 3.3-7%, 3.4-7%, 3.5-7%, 3.6-7%, 3.7-7%, 3.8-7%, 3.9-7%, 4-7%, 4.1-7%, 4.2-7%, 4.3-7%, 4.4-7%, 4.5-7%, 4.6-7%, 4.7-7%, 4.8-7%, 4.9-7%, 5-7%, 5.1-7%, 5.2-7%, 5.3-7%, 5.4-7%, 5.5-7%, 5.6-7%, 5.7-7%, 5.8-7%, 5.9-7%, 6-7%, 6.1-7%, 6.2-7%, 6.3-7° %, 6.4-7%, 6.5-7%, 6.6-7%, 6.7-7%, 6.8-7%, 6.9-7%, 0-7.5%, 0.1-7.5%, 0.2-7.5%, 0.3-7.5%, 0.4-7.5%, 0.5-7.5%, 0.6-75%, 0.7-7.5%, 0.8-7.5%, 0.9-7.5%, 1-7.5%, 1.1-7.5%, 1.2-7.5%, 1.3-7.5%, 1.4-7.5%, 1.5-7.5%, 1.6-7.5%, 1.7-7.5%, 1.8-7.5%, 1.9-7.5%, 2-7.5%, 2.1-7.5%, 2.2-7.5%, 2.3-7.5%, 2.4-7.5%, 2.5-7.5%, 2.6-7.5%, 2.7-7.5%, 2.8-7.5%, 2.9-7.5%, 3-7.5%, 3.1-7.5%, 3.2-7.5%, 3.3-7.5%, 3.4-7.5%, 3.5-7.5%, 3.6-7.5%, 3.7-7.5%, 3.8-7.5%, 3.9-7.5%, 4-7.5%, 4.1-7.5%, 4.2-7.5%, 4.3-7.5%, 4.4-7.5%, 4.5-7.5%, 4.6-7.5%, 4.7-7.5%, 4.8-7.5%, 4.9-7.5%, 5-7.5%, 5.1-7.5%, 5.2-7.5%, 5.3-7.5%, 5.4-7.5%, 5.5-7.5%, 5.6-7.5%, 5.7-7.5%, 5.8-7.5%, 5.9-7.5%, 6-7.5%, 6.1-7.5%, 6.2-7.5%, 6.3-7.5%, 6.4-7.5%, 6.5-7.5%, 6.6-7.5%, 6.7-7.5%, 6.8-7.5%, 6.9-7.5%, 7-7.5%, 7.1-7.5%, 7.2-7.5%, 7.3-7.5%, 7.4-7.5%, 0-8%, 0.1-8%, 0.2-8%, 0.3-8%, 0.4-8%, 0.5-8%, 0.6-8%, 0.7-8%, 0.8-8%, 0.9-8%, 1-8%, 1.1-8%, 1.2-8%, 1.3-8%, 1.4-8%, 1.5-8%, 1.6-8%, 1.7-8%, 1.8-8%, 1.9-8%, 2-8%, 2.1-8%, 2.2-8%, 2.3-8%, 2.4-8%, 2.5-8%, 2.6-8%, 2.7-8%), 2.8-8%, 2.9-8%, 3-8%, 3.1-8%, 3.2-8%, 3.3-8%, 3.4-8%, 3.5-8%, 3.6-8%, 3.7-8%, 3.8-8%, 3.9-8%, 4-8%, 4.1-8%, 4.2-8%, 4.3-8%, 4.4-8%, 4.5-8%, 4.6-8%, 4.7-8%, 4.8-8%, 4.9-8%, 5-8%, 5.1-8%, 5.2-8%, 5.3-8%, 5.4-8%, 5.5-8%, 5.6-8%, 5.7-8%, 5.8-8%, 5.9-8%, 6-8%, 6.1-8%, 6.2-8%, 6.3-8%, 6.4-8%, 6.5-8%, 6.6-8%, 6.7-8%, 6.8-8%, 6.9-8%, 7-8%, 7.1-8%, 7.2-8%, 7.3-8%, 7.4-8%, 7.5-8%, 7.6-8%, 7.7-8%, 7.8-8%, 7.9-8%, 0-8.5%, 0.1-8.5%, 0.2-8.5%, 0.3-8.5%, 0.4-8.5%, 0.5-8.50%, 0.6-8.5%, 0.7-8.5%, 0.8-8.5%, 0.9-8.5%, 1-8.5%, 1.1-8.5%, 1.2-8.5%, 1.3-8.5%, 1.4-8.5%, 1.5-8.5%, 1.6-8.5%, 1.7-8.5%, 1.8-8.5%, 1.9-8.5%, 2-8.5%, 2.1-8.5%, 2.2-8.5%, 2.3-8.5%, 2.4-8.5%, 2.5-8.5%, 2.6-8.5%, 2.7-8.5%, 2.8-8.5%, 2.9-8.5%, 3-8.5%, 3.1-8.5%, 3.2-8.5%, 3.3-8.5%, 3.4-8.5%, 3.5-8.5%, 3.6-8.5%, 3.7-8.5%, 3.8-8.5%, 3.9-8.5%, 4-8.5%, 4.1-8.5%, 4.2-8.5%, 4.3-8.5%, 4.4-8.5%, 4.5-8.5%, 4.6-8.5%, 4.7-8.5%, 4.8-8.5%, 4.9-8.5%, 5-8.5%, 5.1-8.5%, 5.2-8.5%, 5.3-8.5%, 5.4-8.5%, 5.5-8.5%, 5.6-8.5%, 5.7-8.5%, 5.8-8.5%, 5.9-8.5%, 6-8.5%, 6.1-8.5%, 6.2-8.5%, 6.3-8.5%, 6.4-8.5%, 6.5-8.5%, 6.6-8.5%, 6.7-8.5%, 6.8-8.5%, 6.9-8.5%, 7-8.5%, 7.1-8.5%, 7.2-8.5%, 7.3-8.5%, 7.4-8.5%, 7.5-8.5%, 7.6-8.5%, 7.7-8.5%, 7.8-8.5%, 7.9-8.5%, 8-8.5%, 8.1-8.5%, 8.2-8.5%, 8.3-8.5%, 8.4-8.5%, 0-9%, 0.1-9%, 0.2-9%, 0.3-9%, 0.4-9%, 0.5-9%, 0.6-9%, 0.7-9%, 0.8-9%, 0.9-9%, -9%, 1.1-9%, 1.2-9%, 1.3-9%, 1.4-9%, 1.5-9%, 1.6-9%, 1.7-9%, 1.8-9%, 1.9-9%, 2-9%, 2.1-9%, 2.2-9%, 2.3-9%, 2.4-9%, 2.5-9%, 2.6-9%, 2.7-9%, 2.8-9%, 2.9-9%, 3-9%, 3.1-9%, 3.2-9%, 3.3-9%, 3.4-9%, 3.5-9%, 3.6-9%, 3.7-9%, 3.8-9%, 3.9-9%, 4-9%, 4.1-9%, 4.2-9%, 4.3-9%, 4.4-9%, 4.5- 9%, 4.6-9%, 4.7-9%, 4.8-9%, 4.9-9%, 5-9%, 5.1-9%, 5.2-9%, 5.3-9%, 5.4-9%, 5.5-9°%, 5.6-9%, 5.7-9%, 5.8-9%, 5.9-9%, 6-9%, 6.1-9%, 6.2-9%, 6.3-9%, 6.4-9%, 6.5-9%, 6.6-9%, 6.7-9%, 6.8-9%, 6.9-9%, 7-9%, 7.1-9%, 7.2-9%, 7.3-9%, 7.4-9%, 7.5-9%, 7.6-9%, 7.7-9%, 7.8-9%, 7.9-9%, 8-9%, 8.1-9%, 8.2-9%, 8.3-9%, 8.4-9%, 8.5-9%, 8.6-9%, 8.7-9%, 8.8-9%, 8.9-9%, 0-9.5%, 0.1-9.5%, 0.2-9.5%, 0.3-9.5%, 0.4-9.5%, 0.5-9.5%, 0.6-9.5%, 0.7-9.5%, 0.8-9.5%, 0.9-9.5%, 1-9.5%, 1.1-9.5%, 1.2-9.5%, 1.3-9.5%, 1.4-9.5%, 1.5-9.5%, 1.6-9.5%, 1.7-9.5%, 1.8-9.5%, 1.9-9.5%, 2-9.5%, 2.1-9.5%, 2.2-9.5%, 2.3-9.5%, 2.4-9.5%, 2.5-9.5%, 2.6-9.5%, 2.7-9.5%, 2.8-9.5%, 2,9-9.5%, 3-9.5%, 3.1-9.5%, 3.2-9.5%, 3.3-9.5%, 3.4-9.5%, 3.5-9.5%, 3.6-9.5%, 3.7-9.5%, 3.8-9.5%, 3.9-9.5%, 4-9.5%, 4.1-9.5%, 4.2-9.5%, 4.3-9.5%, 4.4-9.5%, 4.5-9.5%, 4.6-9.5%, 4.7-9.5%, 4.8-9.5%, 4.9-9.5%, 5-9.5%, 5.1-9.5%, 5.2-9.5%, 5.3-9.5%, 5.4-9.5%, 5.5-9.5%, 5.6-9.5%, 5.7-9.5%, 5.8-9.5%, 5.9-9.5%, 6-9.5%, 6.1-9.5%, 6.2-9.5%, 6.3-9.5%, 6.4 -9.5%, 6.5-9.5%, 6.6-9.5%, 6.7-9.5%, 6.8-9.5%, 6.9-9.5%, 7-9.5%, 7.1-9.5%, 7.2-9.5%, 7.3-9.5%, 7.4-9.5%, 7.5-9.5%, 7.6-9.5%, 7.7-9.5%, 7.8-9.5%, 7.9-9.5%, 8-9.5%, 8.1-9.5%, 8.2-9.5%, 8.3-9.5%, 8.4-9.5%, 8.5-9.5%, 8.6-9.5%, 8.7-9.5%, 8.8-9.5%, 8.9-9.5%, 9-9.5%, 9.1-9.5%, 9.2-9.5%, 9.3-9.5%, 9.4-9.5%, 0-10%, 0.1-10%, 0.2-10%, 0.3-10%, 0.4-10%, 0.5-10%, 0.6-10%, 0.7-10%, 0.8-10%, 0.9-10%, 1-10%, 1.1-10%, 1.2-10%, 1.3-10%, 1.4-10%, 1.5-10%, 1.6-10%, 1.7-10%, 1.8-10%, 1.9-10%, 2-10%, 2.1-10%, 2.2-10%, 2.3-10%, 2.4-10%, 2.5-10%, 2.6-10%, 2.7-10%, 2.8-10%, 2.9-10%, 3-10%, 3.1-10%, 3.2-10%, 3.3-10%, 3.4-10%, 3.5-10%, 3.6-10%, 3.7-10%, 3.8-10%, 3.9-10%, 4-10%, 4.1-10%, 4.2-10%, 4.3-10%, 4.4-10%, 4.5-10%, 4.6-10%, 4.7-10%, 4.8-10%, 4.9-10%, 5-10%, 5.1-10%, 5.2-10%, 5.3-10%, 5.4-10%, 5.5-10%, 5.6-10%, 5.7-10%, 5.8-10%, 5.9-10%, 6-10%, 6.1-10%, 6.2-10%, 6.3-10%, 6.4-10%, 6.5-10%, 6.6-10%, 6.7-10%, 6.8-10%, 6.9-10%, 7-10%, 7.1-10%, 7.2-10%, 7.3-10%, 7.4-10%, 7.5-10%, 7.6-10%, 7.7-10%, 7.8-10%, 7.9-10%, 8-10%, 8.1-10%, 8.2-10%, 8.3-10%, 8.4-10%, 8.5-10%, 8.6-10%, 8.7-10%, 8.8-10%, 8.9-10%, 9-10%, 9.1-10%, 9.2-10%, 9.3-10%, 9.4-10%, 9.5-10%, 9.6-10%, 9.7-10%, 9.8-10%, or 9.9-10% w/v.

In some embodiments, the formulation may include 0-10% w/v of sucrose.

In some embodiments, the formulation may include 1% w/v of sucrose.

In some embodiments, the formulation may include 2% w/v of sucrose.

In some embodiments, the thnnulation may include 3% w/v of sucrose.

In some embodiments, the formulation may include 4% w/v of sucrose.

In some embodiments, the formulation may include 5% w/v of sucrose,

In some embodiments, the formulation may include 6% w/v of sucrose.

In some embodiments, the formulation may include 7% w/v of sucrose.

In some embodiments, the formulation may include 8% w/v of sucrose.

In some embodiments, the formulation may include 9% w/v of sucrose.

In some embodiments, the thnnulation may include 10% w/v of sucrose.

### Buffering Agents

In some embodiments, formulations of pharmaceutical compositions described herein may comprise a buffering agent to maintain the acidity (pH) of the solution near a desired value. In some embodiments, formulations described herein have a pH within the range of 7.0 to 8.5. The formulations of pharmaceutical compositions described herein may have a pH of 7.0, 7.1 7.2, 7.3, 7.4, 7.5, 7.6, 7.7, 7.8, 7.9, 8, 8.1, or 8.2. In some embodiments, formulations of pharmaceutical compositions described herein may have a pH from 7.2-8.2, 7.2-7.6, 7.3-7.7, or 7.8-8.2. In some embodiments, the pH is determined when the formulation is at 5° C. In some embodiments, the pH is determined when the formulation is at 25° C., Suitable buffering agents may include, hut not limited to, Tris HCl, Tris base, sodium phosphate (monosodium phosphate and/or disodium phosphate), potassium phosphate (monopotassium phosphate and/or dipotassium phosphate), histidine, boric acid, citric acid, glycine, HEPES (4-(2-hydroxyethyl)-1-piperazineethanesulfonic acid), and MOPS (3-(N-morpholino)propanesulfonic acid).

Concentration of buffering agents in the formulation may be between 1-50 mM, between 1-25 mM, between 5-30 mM, between 5-20 mM, between 5-15 mM, between 10-40 mM, or between 15-30 mM. Concentration of buffering agents in the formulation may be about 1 mM, 5 mM, 7.5 mM, 10 mM, 12.5 mM, 15 mM, 20 mM, 25 mM, 30 mM, 35 mM, 40 mM, or 50 mM.

In some embodiments, the formulation may include, but is not limited to, phosphate-buffered saline (PBS). As a non-limiting example, the PBS may include sodium chloride, potassium chloride, disodium phosphate, monopotassium phosphate, and distilled water. In some instances, the PBS does not contain potassium or magnesium. In other instances, the PBS contains calcium and magnesium.

In some embodiments, buffering agents used in the formulations of pharmaceutical compositions described herein may comprise sodium phosphate (monosodium phosphate and/or disodium phosphate). As a non-limiting example, sodium phosphate may be adjusted to a pH (at 5° C.) within the range of 7.4±0.2. In some embodiments, buffering agents used in the formulations of pharmaceutical compositions described herein may comprise Tris base. Tris base may be adjusted with hydrochloric acid to any pH within the range of 7.1 and 9.1. As a non-limiting example, Tris base used in the formulations described herein may be adjusted to 8.0±0.2. As a non-limiting example, Tris base used in the formulations described herein may be adjusted to 7.5±0.2.

In some embodiments, buffering agents in the formulation may be hydrochloric acid. Hydrochloric acid may be used alone or with other buffering agents to adjust the pH of the formulation,

In some embodiments, the concentration of hydrochloric acid in a formulation may be, but is not limited to, 0.1 mM, 0.2 mM, 0.3 mM, 0.4 mM, 0.5 mM, 0.6 mM, 0.7 mM, 0.8 mM, 0.9 mM, 1 mM, 1.1 mM, 1.2 mM, 1.3 mM, 1.4 mM, 1.5 mM, 1.6 mM, 1.7 mM, 1.8 mM, 1.9 mM, 2 mM, 2.1 mM, 2.2 mM, 2.3 mM, 2.4 mM, 2.5 mM, 2.6 mM, 2.7 mM, 2.8 mM, 2.9 mM, 3 mM, 3.1 mM, 3.2 mM, 3.3 mM, 3.4 mM, 3.5 mM, 3.6 mM, 3.7 mM, 3.8 mM, 3.9 mM, 4 mM, 4.1 mM, 4.2 mM, 4.3 mM, 4.4 mM, 4.5 mM, 4.6 mM, 4.7 mM, 4.8 mM, 4.9 mM, 5 mM, 5.1 mM, 5.2 mM, 5.3 mM, 5.4 mM, 5.5 mM, 5.6 mM, 5.7 mM, 5.8 mM, 5.9 mM, 6 mM, 6.1 mM, 6.2 mM, 6.3 mM, 6.4 mM, 6.5 mM, 6.6 mM, 6.7 mM, 6.8 mM, 6.9 mM, 7 mM, 7.1 mM, 7.2 mM, 7.3 mM, 7.4 mM, 7.5 mM, 7.6 mM, 7.7 mM, 7.8 mM, 7.9 mM, 8 mM, 8.1 mM, 8.2 mM, 8.3 mM, 8.4 mM, 8.5 mM, 8.6 mM, 8.7 mM, 8.8 mM, 8.9 mM, 9 mM, 9.1 mM, 9.2 mM, 9.3 mM, 9.4 mM, 9.5 mM, 9.6 mM, 9.7 mM, 9.8 mM, 9.9 mM, 10 mM, 10.1 mM, 10.2 mM, 10.3 mM, 10.4 mM, 10.5 mM, 10.6 mM, 10.7 mM, 10.8 mM, 10.9 mM, 11 mM, 11.1 mM, 11.2 mM, 11.3 mM, 11.4 mM, 11.5 mM, 11.6 mM, 11.7 mM, 11.8 mM, 11.9 mM, 12 mM, 12.1 mM, 12.2 mM, 12.3 mM, 12.4 mM, 12.5 mM, 12.6 mM, 12.7 mM, 12.8 mM, 12.9 mM, 13 mM, 13.1 mM, 13.2 mM, 13.3 mM, 13.4 mM, 13.5 mM, 13.6 mM, 13.7 mM, 13.8 mM, 13.9 mM, 14 mM, 14.1 mM, 14.2 mM, 14.3 mM, 14.4 mM, 14.5 mM, 14.6 mM, 14.7 mM, 14.8 mM, 14.9 mM or 15 mM.

The formulation may include hydrochloric acid in a range of 0-0.5 mM, 0.1-0.6 mM, 0.2-0.7 mM, 0.3-0.8 mM, 0.4-0.9 mM, 0.5-1 mM, 0.6-1.1 mM, 0.7-1.2 mM, 0.8-1.3 mM, 0.9-1.4 mM, 1-1.5 mM, 1.1-1.6 mM, 1.2-1.7 mM, 1.3-1.8 mM, 1.4-1.9 mM, 1.5-2 mM, 1.6-2.1 mM, 1.7-2.2 mM, 1.8-2.3 mM, 1.9-2.4 mM, 2-2.5 mM, 2.1-2.6 mM, 2.2-2.7 mM, 2.3-2.8 mM, 2.4-2.9 mM, 2.5-3 mM, 2.6-3.1 mM, 2.7-3.2 mM, 2.8-3.3 mM, 2.9-3.4 mM, 3-3.5 mM, 3.1-3.6 mM, 3.2-3.7 mM, 3.3-3.8 mM, 3.4-3.9 mM, 3.5-4 mM, 3.6-4.1 mM, 3.7-4.2 mM, 3.8-4.3 mM, 3.9-4.4 mM, 4-4.5 mM, 4.1-4.6 mM, 4.2-4.7 mM, 4.3-4.8 mM, 4.4-4.9 mM, 4.5-5 mM, 4.6-5.1 mM, 4.7-5.2 mM, 4.8-5.3 mM, 4.9-5.4 mM, 5-5.5 mM, 5.1-5.6 mM, 5.2-5.7 mM, 5.3-5.8 mM, 5.4-5.9 mM, 5.5-6 mM, 5.6-6.1 mM, 5.7-6.2 mM, 5.8-6.3 mM, 5.9-6.4 mM, 6-6.5 mM, 6.1-6.6 mM, 6.2-6.7 mM, 6.3-6.8 mM, 6.4-6.9 mM, 6.5-7 mM, 6.6-7.1 mM, 6.7-7.2 mM, 6.8-7.3 mM, 6.9-7.4 mM, 7-7.5 mM, 7.1-7.6 mM, 7.2-7.7 mM, 7.3-7.8 mM, 7.4-7.9 mM, 7.5-8 mM, 7.6-8.1 mM, 7.7-8.2 mM, 7.8-8.3 mM, 7.9-8.4 mM, 8-8.5 mM, 8.1-8.6 mM, 8.2-8.7 mM, 8.3-8.8 mM, 8.4-8.9 mM, 8.5-9 mM, 8.6-9.1 mM, 8.7-9.2 mM, 8.8-9.3 mM, 8.9-9.4 mM, 9-9.5 mM, 9.1-9.6 mM, 9.2-9.7 mM, 9.3-9.8 mM, 9.4-9.9 mM, 9.5-10 mM, 9.6-10.1 mM, 9.7-10.2 mM, 9.8-10.3 mM, 9.9-10.4 mM, 10-10.5 mM, 10.1-10.6 mM, 10.2-10.7 mM, 10.3-10.8 mM, 10.4-10.9 mM,10.5-11 mM, 10.6-11.1 mM, 10.7-11.2 mM, 10.8-11.3 mM, 10.9-11.4 mM, 11-11.5 mM, 11.1-11.6 mM, 11.2-11.7 mM, 11.3-11.8 mM, 11.4-11.9 mM, 11.5-12 mM, 11.6-12.1 mM, 11.7-12.2 mM, 11.8-12.3 mM, 11.9-12.4 mM, 12-12.5 mM, 12.1-12.6 mM, 12.2-12.7 mM, 12.3-12.8 mM, 12.4-12.9 mM, 12.5-13 mM, 12.6-13.1 mM, 12.7-13.2 mM, 12.8-13.3 mM, 12.9-13.4 mM, 13-13.5 mM, 13.1-13.6 mM, 13.2-13.7 mM, 13.3-13.8 mM, 13.4-13.9 mM, 13.5-14 mM, 13.6-14.1 mM, 13.7-14.2 mM, 13.8-14.3 mM, 13.9-14.4 mM, 14-14.5 mM, 14.1-14.6 mM, 14.2-14.7 mM, 14.3-14.8 mM, 14.4-14.9 mM, 14.5-15 mM, 0-1 mM, 1-2 mM, 2-3 mM, 3-4 mM, 4-5 mM, 5-6 mM, 6-7 mM, 7-8 mM, 8-9 mM, 9-10 mM, 10-11 mM, 11-12 mM, 12-13 mM, 13-14 mM, 14-15 mM, 15-16 mM, 0-2 mM, 1-3 mM, 2-4 mM, 3-5 mM, 4-6 mM, 5-7 mM, 6-8 mM, 7-9 mM, 8-10 mM, 9-11 mM, 10-12 mM, 11-13 mM, 12-14 mM, 13-15 mM, 0-3 mM, 1-4 mM, 2-5 mM, 3-6 mM, 4-7 mM, 5-8 mM, 6-9 mM, 7-10 mM, 8-11 mM, 9-12 mM, 10-13 mM, 11-14 mM, 12-15 mM, 0-4 mM, 1-5 mM, 2-6 mM, 3-7 mM, 4-8 mM, 5-9 mM, 6-10 mM, 7-11 mM, 8-12 mM, 9-13 mM, 10-14 mM, 11-15 mM, 0-5 mM, 1-6 mM, 2-7 mM, 3-8 mM, 4-9 mM, 5-10 mM, 6-11 mM, 7-12 mM, 8-13 mM, 9-14 mM, 10-15 mM, 0-6 mM, 1-7 mM, 2-8 mM, 3-9 mM, 4-10 mM, 5-11 mM, 6-12 mM, 7-13 mM, 8-14 mM, 9-15 mM, 0-7 mM, 1-8 mM, 2-9 mM, 3-10 mM, 4-1.1 mM, 5-12 mM, 6-13 mM, 7-14 mM, 8-15 mM, 0-8 mM, 1-9 mM, 2-10 mM, 3-11 mM, 4-12 mM, 5-13 mM, 6-14 mM, 7-15 mM, 0-9 mM, 1-10 mM, 2-11 mM, 3-12 mM, 4-13 mM, 5-14 mM, 6-15 mM,0-10 mM, 1-11 mM, 2-12 mM, 3-13 mM, 4-14 mM, 5-15 mM, 0-11 mM, 1-12, mM, 2-13 mM, 3-14 mM, 4-15 mM, 0-12 mM, 1-13 mM, 2-14 mM, 3-15 mM,0-13 mM, 1-14 mM, 2-15 mM, 0-14 mM, 1-15 mM, or 0-15 mM.

In some embodiments, the formulation may include 0-10 mM of hydrochloric acid.

In some embodiments, the formulation may include 6.2-6.3 mM of hydrochloric acid.

in some embodiments, the formulation may include 8.9-9 mM of hydrochloric acid.

In some embodiments, the formulation may include 6.2 mM of hydrochloric acid.

In some embodiments, the formulation may include 6.3 mM of hydrochloric acid.

In some embodiments, the formulation may include 8.9 mM of hydrochloric acid.

In some embodiments, the formulation may include 9 mM of hydrochloric acid.

### Surfactants

In some embodiments, formulations of pharmaceutical compositions described herein may comprise a surfactant. Surfactants may help control shear forces in suspension cultures. Surfactants used herein may be anionic, zwitterionic, or non-ionic surfactants and may include those known in the art that are suitable for use in pharmaceutical formulations. Examples of anionic surfactants include, but are not limited to, sulfate, sulfonate, phosphate esters, and carboxylates. Examples of nonionic surfactants include, but are not limited to, ehoxylates, fatty alcohol ethoxylates, alkylphenol ethoxylates (e.g., nonoxynols, Triton X-100), fatty acid ethoxylates, ethoxylated amines and/or fatty acid amides (e.g., polyethoxylated tallow amine, cocamide monoethanolamine, cocamide diethanolamine), ethylene oxide/propylene oxide copolymer (e.g., Poloxamers such as Pluronic® F-68 or F-127), esters of fatty acids and polyhydric alcohols, fatty acid alkanolatnides, ethoxylated aliphatic acids, ethoxylated aliphatic alcohols, ethoxylated sorbitol fatty acid esters, ethoxylated glycerides, ethoxylated block copolymers with. EDTA (ethylene diaminetetraacetic acid), ethoxylated cyclic ether adducts, ethoxylated amide and imidazoline adducts, ethoxylated amine adducts, ethoxylated mercaptan adducts, ethoxylated condensates with alkyl phenols, ethoxylated nitrogen-based hydrophobes, ethoxylated polyoxypropylenes, polymeric silicones, fluorinated surfactants, and polymerizable surfactants. Examples of zwitterionic surfactants include, but are not limited to, alkylamido betaines and amine oxides thereof, alkyl betaines and amine oxides thereof, sulth betaines, hydroxy sulfo betaines, amphoglycinates, amphopropionates, balanced amphopolycarboxyglycinates, and alkyl polyaminoglycinates. Proteins have the ability of being charged or uncharged depending on the pH; thus, at the right pH, a protein, preferably with a pi of about 8 to 9, such as modified Bovine Serum Albumin or chymnotrypsinogen, could function as a zwitterionic surfactant. Various mixtures of surfactants can be used if desired.

In some embodiments, surfactants used in the formulations of pharmaceutical compositions described herein includes at least one ethylene oxide/propylene copolymer.

In some embodiments, the formulation may include Poloxamer. In some embodiments, the formulation may include Poloxamer in a range of 0.00001%-0.0001%, 0.00001%-0.001%, 0.00001%-0.01%, 0.00001%-0.1%, 0.00001%-1%, 0.0001%-0.001%, 0.0001%-0.01%, 0.0001%-0.1%, 0.0001%-1%, 0.001%-0.01%, 0.001%-0.1%, 0.001%-1%, 0.01%-0.1%, 0.01%-1%, or 0.1-1% w/v.

In some embodiments, the formulation may include 0.001% w/v oxamer.

In some embodiments, the formulation may include Poloxamer 188 (e.g., Pluronic® F-68). In some embodiments, the formulation may include Poloxamer 188 at a concentration of 0.00001%, 0.0001%, 0.001%, 0.01%), 0.1%, or 1% w/v.

In some embodiments, the formulation may include Poloxamer 188 in a range of 0.00001%-0.0001%, 0.00001%-0.001%, 0.00001%-0.01%, 0.00001%-0.1%, 0.00001%-1%, 0.0001%-0.001%, 0.0001%-0.01%, 0.0001%-0.1%, 0.0001%-1%, 0.001%-0.01%, 0.001%-0.1%, 0.001%-1%, 0.01%-0.1%, 0.01%-1%, or 0.1-1% w/v.

In some embodiments, the formulation may include 0.001% w/v Poloxamer 188.

In some embodiments, the formulation may include Pluronic rt F-68. In some embodiments, the formulation may include Pluronic® F-68 at a concentration of 0.00001%, 0.0001%, 0.001%, 0.01%, 0.1%, or 1% w/v.

In some embodiments, the formulation may include Pluronic ® F-68 in a range of 0.00001%-0.0001%, 0.00001%-0.001%, 0.00001%-0.01%, 0.00001%-0.1%, 0.00001%-1%, 0.0001%-0.001%, 0.0001%-0.01%, 0.0001%-0.1%, 0.0001%-1%, 0.001%-0.01%, 0.001%-0.1%, 0.001%-1%, 0.01%-0.1%, 0.01%-1%, or 0.1-1% w/v.

In some embodiments, the formulation may include 0.001% w/v Pluronic rt F-68.

### Osmolality

In some embodiments, the formulation may be optimized for a specific The osmolality of the formulation may be, but is not limited to, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456 457, 458, 459, 460, 461, 462, 463, 464, 465, 466, 467, 468, 469, 470, 471, 472, 473, 474, 475, 476, 477, 478, 479 480, 481, 482, 483, 484, 485, 187, 488, 489, 490, 491, 492, 493, 494, 495, 496, 497, 498, 499, or 500 mOsm/kg (milliosmoles/kg).

In some embodiments, the formulation may be optimized for a specific range of osmolality. The range may be, but is not limited to, 350-360, 360-370, 370-380, 380-390, 390-400, 400-410, 410-420, 420-430, 430-440, 440-450, 450-460, 460-470, 470-480, 480-490, 490-500, 350-370, 360-380, 370-390, 380-400, 390-410, 400-420, 410-430, 420-440, 430-450, 440-460, 450-470, 460-480, 470-490, 480-500, 350-375, 375-400, 400-425, 425-450, 450-475, 475-500, 350-380, 360-390, 370-400, 380-410, 390-420, 400-430, 410-440, 420-450, 430-460, 440-470, 450-480, 460-490, 470-500, 350-390, 360-400, 370-410, 380-420, 390-430, 400-440, 410-450, 420-460, 430-470, 440-480, 450-490, 460-500, 350-400, 360-410, 370-420, 380-430, 390-440, 400-450, 410-460, 420-470, 430-480, 440-490, 450-500, 350-410, 360-420, 370-430, 380-440, 390-450, 400-460, 410-470, 420-480, 430-490, 440-500, 350-420, 360-430, 370-440, 380-450, 390-460, 400-470, 410-480, 420-490, 430-500, 350-430, 360-440, 370-450, 380-460, 390-470, 400-480, 410-490, 420-500, 350-440, 360-450, 370-460, 380-470, 390-480, 400-490, 410-500, 350-450, 360-460, 370-470, 380-480, 390-490, 400-500, 350-460, 360-470, 370-480, 380-490, 390-500, 350-470, 360-480, 370-490, 380-500, 350-480, 360-490, 370-500, 350-490, 360-500, or 350-500 mOsm/kg.

In some embodiments, the osmolality of the formulation is between 350-500 mOsm/kg.

In some embodiments, the osmolality of the formulation is between400-500 mOsm/kg.

In some embodiments, the osmolality of the formulation is between 400-480 mOsm/kg.

In some embodiments, the maximum osmolality of the formulation is about 500 mOsm/kg.

In some embodiments, the osmolality for convection enhanced delivery (CED) infusion into the brain is about 400-480 mOsm/kg.

In some embodiments, the maximum osmolality for convection enhanced delivery (CED) infusion into the brain is about 400-480 mOsm/kg.

In some embodiments, the maximum osmolality for convection enhanced delivery (CED) infusion into the brain is about 500 mOsm/kg.

### Concentration of AAV Particle

In some embodiments, the concentration of AAV particle in the formulation may be between about 1×10⁶ VG/mL and about 1×10¹⁶ VG/mL. As used herein, “VG/mL,” represents vector genomes (VG) per milliliter (mL). VG/mi, also may describe genome copy per milliliter or DNase resistant particle per milliliter.

In some embodiments, the formulation may include an AAV particle concentration of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷ 8×10⁷, 9×10⁷, 1×10⁸, 2×10⁸, 3×10⁸, 4×10⁸, 5×10⁵, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 2.1×10¹¹, 2.2×10¹¹, 2.4×10¹¹, 2.5×10¹¹, 2.6×10¹¹, 2.7×10¹¹, 2.9×10¹¹, 3×10¹¹, 4×10¹¹, 5×10¹¹, 6×10¹¹, 7×10¹¹, 7.1×10¹¹, 7.2×10¹¹, 7.3×10¹¹, 7.4×10¹¹, 7.5×10¹¹, 7.6×10¹¹, 7.7×10¹¹, 7.8×10¹¹, 7.9×10¹¹, 8×10¹¹, 9×10¹¹, 1×10¹², 1.1×10¹², 1.2×10¹², 1.3×10¹², 1.4×10¹², 1.5×10¹², 1.6×10¹², 1.7×10¹², 1.8×10¹², 1.9×10¹², 2×10¹², 2.1×10¹², 2.2×10¹², 2.3×10¹², 2.4×10¹², 2.5×10¹², 2.6×10¹², 2.7×10¹², 2.8×10¹², 2.9×10¹², 3×10¹², 4×10¹², 4.1×10¹², 4.2×10¹², 4.3×10¹², 4.4×10¹², 4.5×10¹², 4.6×10¹², 4.7×10¹², 4.8×10¹², 4.9×10¹², 5×10 6×10¹², 7×10¹², 7.1×10¹², 7.2×10¹², 7.3×10¹², 7.4×10¹², 7.5×10¹², 7.6×10¹², 7.7×10¹², 7.8×10¹², 7.9×10¹², 8×10¹², 8.1×10¹², 8.2×10¹², 8.3×10¹², 8.4×10¹², 8.5×10¹², 8.6×10¹², 8.7×10¹², 8.8×10¹², 8.9×10¹², 9×10¹², 1×10¹³, 1.1×10¹³, 1.2×10¹³ 1.3×10¹³, 1.4×10¹³, 1.5×10¹³, 1.6×10¹³, 1.7×10¹³ 1.8×10¹³ 1.9×10¹³, 2×10¹³, 2.7×10¹³, 3×10¹³, 3.1×10¹³, 3.2×10¹³, 3.3×10¹³, 3.4×10¹³, 3.5×10¹³, 3.6×10¹³, 3.7×10¹³, 3.8×10¹³, 3.9×10¹³, 4×10¹³, 5×10¹³, 6×10¹³, 6.7×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG/mL.

In some embodiments, the concentration of AAV particles in the formulations may be between 1×10¹¹ and 5×10¹³, between 1×10¹² and 5×10¹², between 2×10¹² and 1×10¹³between 5×10¹² and 1×10¹³, between 1×10¹³ and 2×10¹³, between 2×10¹³ and 3×10¹³, between 2×10¹³ and 2.5×10¹³, between 2.5×10¹³ and 3×10¹³, or no more than 5×10¹³ VG/mL.

In some embodiments, the concentration of AAV particle in the formulation is 2.7×10¹¹ VG/mL,

In some embodiments, the concentration of AAV particle in the formulation is 9×10¹¹ VG/mL.

In some embodiments, the concentration of AAV particle in the formulation is 2.7×10¹²VG/mL.

In some embodiments, the concentration of AAV particle in the formulation is 4×10¹² VG/mL.

In some embodiments, the concentration of AAV particle in the formulation is 7.9×10¹² VG/mL.

In some embodiments, the concentration of AAV particle in the formulation is 1.0×10¹³ VG/mL.

In some embodiments, the concentration of AAV particle in the formulation is 2.2×10¹³ VG/mL.

In some embodiments, the concentration of AAV particle in the formulation is 2.7×10¹³ VG/mL,

In some embodiments, the concentration of AAV particle in the formulation is 3.5×10¹³ VG/ML.

In some embodiments, the concentration of AAV particle in the formulation may be between about 1×10⁶ total capsid/mL and about 1×10¹⁶ total capsid/mL. In some embodiments, delivery may comprise a composition concentration of about 1×10⁶, 2×10⁶, 3×10⁶, 4×10⁶, 5×10⁵, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10²², 1×10¹², 1.1×10¹², 1.2×10¹², 1.3×10¹², 1.4×10¹², 1.5×10¹², 1.6×10¹², 1.7×10¹², 1.8×10¹², 1.9×10¹², 2×10¹², 2.1×10¹², 2.2×10¹², 2.3×10¹², 2.4×10¹², 2.5×10¹², 2.6×10¹², 2.7×10¹², 2.8×10¹², 2.9×10¹², 3×10¹², 3.1×10¹², 3.2×10¹², 3.3×10¹², 3.4×10¹², 3.5×10¹², 3.6×10¹², 3.7×10¹², 3.8×10¹², 3.9×10¹², 4×10¹², 4.1×10¹², 4.2×10¹², 4.3×10¹², 4.4×10¹², 4.5×10¹², 4.6×10¹², 4.7×10¹², 4.8×10¹², 4.9×10¹², 5×10¹², 6×10¹², 7×10¹², 8×10¹², 9×10¹², 1×10¹³, 2×10¹³, 2.7×10¹³, 3×10¹³, 4×10¹³, 5×10¹³, 6×10¹³, 6.7×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ total capsid/mL.

### Total Dose of AAV Particle

In some embodiments, the total dose of the AAV particle in the form elation may be between about 1×10⁶ VG and about 1×10¹⁶ VG. In some embodiments, the formulation may include a total dose of AAV particle of about 1×10⁶, 2×10⁶, 3×10⁶, 4×10⁶, 5×10⁶, 6×10⁶, 7×10⁶, 8×10⁶, 9×10⁶, 1.×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 2×10¹¹, 2.2×10¹¹, 2.3×10¹¹, 2.4×10¹¹, 2.5×10¹¹, 2.6×10¹¹, 2.7×10¹¹, 2.8×10¹¹, 2.9×10¹¹, 3×10¹¹, 4×10¹¹, 5×10¹¹, 6×10¹¹, 7×10¹¹, 7.1×10¹¹, 7.2×10¹¹, 7.3×10¹¹, 7.4×10¹¹, 7.5×10¹¹, 7.6×10¹¹, 7.7×10¹¹, 7.8×10¹¹, 7.9×10¹¹, 8×10¹¹, 9×10¹¹, 1×10¹², 1.1×10¹², 1.2×10¹², 1.3×10¹², 1.4×10¹², 1.5×10¹², 1.6×10¹², 1.7×10¹², 1.8×10¹², 1.9×10¹², 2×10¹², 2.1×10¹², 2.2×10¹², 2.3×10¹², 2.4×10¹², 2.5×10¹², 2.6×10¹², 2.7×10¹², 2.8×10¹², 2.9×10¹², 3×10¹², 4×10¹², 4.1×10¹², 4.2×10¹², 4.3×10¹², 4.4×10¹², 4.5×10¹², 4.6×10¹², 4.7×10¹², 4.8×10¹², 4.9×10¹², 5×10¹², 6×10¹², 7×10¹², 7.1×10¹², 7.2×10¹², 7.3×10¹², 7.4×10¹², 7.5×10¹², 7.6×10¹², 7.7×10¹², 7.8×10¹², 7.9×10¹², 8×10¹², 8.1×10¹², 8.2×10¹², 8.3×10¹², 8.4×10¹², 8.5×10¹², 8.6×10¹², 8.7×10¹², 8.8×10¹², 8.9×10¹², 9×10¹², 1×10¹², 1.1×10¹³, 1.2×10¹², 1.3×10¹³, 1.4×10¹³, 1.5×10¹³, 1.6×10¹³, 1.7×10¹³, 1.8×10¹³, 1.9×10¹³, 2×10¹³, 2.7×10¹³, 3×10¹³, 3.1×10¹³, 3.2×10¹³, 3.3×10¹³, 3.4×10¹³, 3.5×10¹³, 3.6×10¹³, 3.7×10¹³, 3.8×10¹³, 3.9×10¹³, 4×10¹³, 5×10¹³, 6×10¹³, 6.7×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG.

In some embodiments, the total dose of AAV particle in the formulations between 1×10¹¹ and 2×10¹⁴ VG.

### Exemplary Formulations

In some embodiments, the formulations may include sodium phosphate, potassium phosphate, sodium chloride, potassium chloride, and optionally a surfactant such as Poloxamer 188 (e.g., Pluronic® F-68). As a non-limiting example, the formulation may include 10 mM sodium phosphate, 2 mM. potassium phosphate, 192 mM sodium chloride, 2.7 mM potassium chloride, and 0.001% (w/v) Poloxamer 188. The formulations may be used to formulate an AAV particle at a concentration of about 2.7×10¹² VG/mL.

In some embodiments, the formulations may include Phosphate Buffered Saline, sucrose and optionally a surfactant such as Poloxamer 188, As a non-limiting example, the formulation may include Phosphate Buffered Saline. 5% sucrose and 0.001% (w/v) Poloxamer 188. The formulations may be used to formulate an AAV particle at a concentration of about 2.2×10¹² VG/mL.

In some embodiments, the formulations may include sodium phosphate, potassium phosphate, sodium chloride, sucrose and optionally a surfactant such as Poloxamer 188. As a non-limiting example, the formulation may include 2.7 mM sodium phosphate, 1.54 mM potassium phosphate, 155 mM sodium chloride, and 5% (w/v) sucrose at pH 7.2 and with an osmolality of 450 mOsm/kg.

In some embodiments, the formulations may include sodium phosphate, potassium phosphate, sodium chloride, sucrose, and optionally a surfactant such as Poloxamer 188. As a non-limiting example, the formulation may include 10 mM sodium phosphate, 1.5 mM potassium phosphate, 95 mM sodium chloride, 7% (w/v) sucrose, and 0.001% (w/v) Poloxamer 188, pH 7.4±0.2 at 5° C. The formulations may be used to formulate an AAV particle at a concentration of about 2.7×10¹³ VG/mL.

In some embodiments, the formulation may include Tris Base, hydrochloric acid, potassium chloride, sodium chloride, sucrose, and optionally a surfactant such as Poloxamer 188. As a non-limiting example, the formulation may include 10 mM Tris Base, 6.3 mM HCl, 1.5 mM Potassium Chloride, 100 mM Sodium Chloride, 7% (w/v) Sucrose, and 0.001% (w/v) Poloxamer 188, pH 8.0±0.2 at 5° C. As another non-limiting example, the formulation may include 10 mM Tris Base, 9 mM HCl, 1.5 mM potassium chloride, 100 mM sodium chloride, 7% (w/v) sucrose, and 0.001% (w/v) Poloxamer 188, pH 7.5±0.2 at 5° C. The formulations may be used to formulate an AAV particle at a concentration of about 2.7×10¹³ VG/mL.

### Delivery

In some embodiments, the AAV particles described herein may be administered or delivered using the methods for the delivery of AAV virions described in European Patent Application No. EP1857552, the contents of which are herein incorporated by reference in its entirety.

In some embodiments, the AAV particles described herein may be administered or delivered using the methods for delivering proteins using AAV vectors described in European Patent Application No, EP2678433, the contents of which are herein incorporated by reference in its entirety.

In some embodiments, the AAV particle described herein may be administered or delivered using the methods for delivering DNA molecules using AAV vectors described in U.S. Pat. No. 5,858,351, the contents of which are herein incorporated by reference in its entirety.

In some embodiments, the AAV particle described herein may be administered or delivered using the methods for delivering DNA to the bloodstream described in U.S. Pat. No. 6,211,163, the contents of which are herein incorporated by reference in its entirety.

In some embodiments, the AAV particle described herein may be administered or delivered using the methods for delivering AAV virions described in U.S. Pat. No. 6,325,998, the contents of which are herein incorporated by reference in its entirety, In some embodiments, the AAV particle described herein may be administered or delivered using the methods for delivering a payload to the central nervous system described in U.S. Pat. No. 7,588,757, the contents of which are herein incorporated. by reference in its entirety.

In some embodiments, the AAV particle described herein may be administered or delivered using the methods for delivering a payload described in U.S. Pat. No. 8,283,151, the contents of which are herein incorporated by reference in its entirety.

In some embodiments, the AAV particle described herein may be administered or delivered using the methods for delivering a payload using a glutamic acid decarboxylase (GAD) delivery vector described in International Patent Publication No. WO2001089583, the contents of which are herein incorporated by reference in its entirety.

In some embodiments, the AAV particle described herein may be administered or delivered using the methods for delivering a payload to neural cells described in International Patent Publication No. WO2012057363, the contents of which are herein incorporated by reference in its entirety.

### Delivery to Cells

The present disclosure provides a method of delivering to a cell or tissue any of the above-described AAV polynucleotides or AAV genomes, comprising contacting the cell or tissue with said AAV polynucleotide or AAV genomes or contacting the cell or tissue with a particle comprising said AAV polynucleotide or AAV genome, or contacting the cell or tissue with any of the described compositions, including pharmaceutical compositions. The method of delivering the AAV polynucleotide or AAV genome to a cell or tissue can be accomplished in vitro, ex vivo, or in vivo.

Introduction into Cells—AAV Particles

The encoded siRNA molecules (e.g., siRNA duplexes) of the present disclosure may be introduced into cells by being encoded by the vector genome (VG) of an AAV particle. These AAV particles are engineered and optimized to facilitate the entry of siRNA molecule into cells that are not readily amendable to transfection. Also, some synthetic AAV particles possess an ability to integrate the shRNA into the cell genome, thereby leading to stable siRNA expression and long-term knockdown of a target gene. In this manner, AAV particles are engineered as vehicles for specific delivery while lacking the deleterious replication and/or integration features found in wild-type virus.

In some embodiments, the encoded siRNA molecules of the present disclosure are introduced into a cell by contacting the cell with an AAV particle comprising a modulatory polynucleotide sequence encoding a siRNA molecule, and a lipophilic carrier. In other embodiments, the siRNA molecule is introduced into a cell by transfecting or infecting the cell with an AAV particle comprising a nucleic acid sequence capable of producing the siRNA molecule when transcribed in the cell. In some embodiments, the siRNA molecule is introduced into a cell by injecting into the cell an AAV particle comprising a nucleic acid sequence capable of producing the siRNA molecule when transcribed in the cell.

In some embodiments, prior to transfection, an AAV particle comprising a nucleic acid sequence encoding the siRNA molecules of the present disclosure may be transfected into cells.

In other embodiments, the AAV particles comprising the nucleic acid sequence encoding the siRNA molecules of the present disclosure may be delivered into cells by electroporation (e.g. U.S. Patent Publication No. 20050014264; the content of which is herein incorporated by reference in its entirety).

Other methods for introducing AAV particles comprising the nucleic acid sequence encoding the siRNA molecules described herein may include photochemical internalization as described in U.S. Patent publication No. 20120264807; the content of which is herein incorporated by reference in its entirety.

In some embodiments, the formulations described herein may contain at least one AAV particle comprising the nucleic acid sequence encoding the siRNA molecules described herein. In some embodiments, the siRNA molecules may target the HTT gene at one target site. In another embodiment, the formulation comprises a plurality of AAV particles, each AAV particle comprising a nucleic acid sequence encoding a siRNA molecule targeting the HTT gene at a different target site. The HTT may be targeted at 2, 3, 4, 5 or more than 5 sites.

In some embodiments, the AAV particles from any relevant species, such as, but not limited to, human, pig, dog, mouse, rat or monkey may be introduced into cells.

In some embodiments, the AAV particles may be introduced into cells which are relevant to the disease to be treated. As a non-limiting example, the disease is HD and the target cells are neurons and astrocytes. As another non-limiting example, the disease is HD and the target cells are medium spiny neurons, cortical neurons and astrocytes.

In some embodiments, the AAV particles may be introduced into cells which have a high level of endogenous expression of the target sequence.

In another embodiment, the AAV particles may be introduced into cells which have a low level of endogenous expression of the target sequence.

In some embodiments, the cells may be those which have a high efficiency of AAV transduction.

### Delivery to Subjects

The present disclosure additionally provides a method of delivering to a subject, including a mammalian subject such as, but not limited to, a patient in need thereof, any of the above-described AAV polynucleotides or AAV genomes comprising administering to the subject said AAV polynucleotide or AAV genome, or administering to the subject a particle comprising said AAV polynucleotide or AAV genome, or administering to the subject any of the described compositions, including pharmaceutical compositions.

The pharmaceutical compositions of AAV particles described herein may be characterized by one or more of bioavailability, therapeutic window and/or volume of distribution.

### III. Administration and Dosing

### Administration

The AAV particles comprising a nucleic acid sequence encoding the siRNA molecules of the present disclosure may be administered by any route which results in a therapeutically effective outcome. These include, but are not limited to, within the parenchyma of an organ such as, but not limited to, a brain (e.g., intraparenchymal), corpus striatum (intrastriatal), enteral (into the intestine), gastroenteral, epidural, oral (by way of the mouth), transdermal, peridural, intracerebral (into the cerebrum), intracerebroventricular (into the cerebral ventricles), subpial (under the pia), epicutaneous (application onto the skin), intradermal, (into the skin itself), subcutaneous (under the skin), nasal administration (through the nose), intravenous (into a vein), intravenous bolus, intravenous drip, intraarterial (into an artery), intramuscular (into a muscle), intracardiac (into the heart), intraosseous infusion (into the bone marrow), intrathecal (into the spinal canal), intraganglionic (into the ganglion), intraperitoneal, (infusion or injection into the peritoneum), intravesical infusion, intravitreal, (through the eye), intracavemous injection (into a pathologic cavity) intracavitary (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), in ear drops, auricular (in or by way of the ear), buccal (directed toward the cheek), conjunctival, cutaneous, dental (to a tooth or teeth), electro-osmosis, endocervical, endosinusial, endotracheal, extracorporeal, hemodialysis, infiltration, interstitial, intra-abdominal, intra-amniotic, intra-articular, intrabiliary, intrabronchial, intrabursal, intracartilaginous (within a cartilage), intracaudal (within the cauda equine), intracisternal (within the cisterna magna cerebellomedularis intracomeal (within the cornea), dental intracornal, intracoronary (within the coronary arteries), intracorporus cavernosum (within the dilatable spaces of the corporus cavernosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the aunts media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique (topical route administration which is then covered by a dressing which occludes the area), ophthalmic (to the external eye), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), soft tissue, subarachnoid, subconjunctival, submucosal, topical, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis or spinal.

In specific embodiments, compositions of AAV particles comprising a nucleic acid sequence encoding the siRNA molecules of the present disclosure may be administered in a way which facilitates the vectors or siRNA molecule to enter the central nervous system and penetrate into medium spiny and/or cortical neurons and/or astrocytes.

In some embodiments, the AAV particles comprising a nucleic acid sequence encoding the siRNA molecules of the present disclosure may be administered by intramuscular injection.

In some embodiments, the AAV particles comprising a nucleic acid sequence encoding the siRNA molecules of the present disclosure may be administered via intraparenchymal injection.

In some embodiments, the AAV particles comprising a nucleic acid sequence encoding the siRNA molecules of the present disclosure may be administered via intraparenchymal injection and intrathecal injection.

In some embodiments, the AAV particles comprising a nucleic acid sequence encoding the siRNA molecules of the present disclosure may be administered via intrastriatal injection.

In some embodiments, the AAV particles comprising a nucleic acid sequence encoding the siRNA molecules of the present disclosure may be administered via intrastriatal injection and another route of administration described herein.

In some embodiments, AAV particles that express siRNA duplexes of the present disclosure may be administered to a subject by peripheral injections (e.g., intravenous) and/or intranasal delivery. It was disclosed in the art that the peripheral administration of AAV particles for siRNA duplexes can be transported to the central nervous system, for example, to the neurons (e.g., U. S. Patent Publication Nos. 20100240739; and 20100130594; the content of each of which is incorporated herein by reference in their entirety).

In other embodiments, compositions comprising at least one AAV particle comprising a nucleic acid sequence encoding the siRNA molecules of the present disclosure may be administered to a subject by intracranial delivery (See, e.g., U. S. Pat. No. 8,119,611; the content of which is incorporated herein by reference in its entirety),

The AAV particle comprising a nucleic acid sequence encoding the siRNA molecules of the present disclosure may be administered in any suitable form, either as a liquid solution or suspension, as a solid form suitable for liquid solution or suspension in a liquid solution. The siRNA duplexes may be formulated with any appropriate and pharmaceutically acceptable excipient.

The AAV particle comprising a nucleic acid sequence encoding the siRNA molecules of the present disclosure may be administered in a “therapeutically effective” amount, i.e., an amount that is sufficient to alleviate and/or prevent at least one symptom associated with the disease, or provide improvement in the condition of the subject.

In some embodiments, the AAV particle may be administered to the CNS in a therapeutically effective amount to improve function and/or survival for a subject with Huntington's Disease (HD). As a non-limiting example, the vector may be administered by direct infusion into the striatum.

In some embodiments, the AAV particle may be administered to a subject (e.g., to the CNS of a subject via intrathecal administration) in a therapeutically effective amount for the siRNA duplexes or dsRNA to target the medium spiny neurons, cortical neurons and/or astrocytes. As a non-limiting example, the siRNA duplexes or dsRNA may reduce the expression of HTT protein or mRNA. As another non-limiting example, the siRNA duplexes or dsRNA can suppress HTT and reduce HTT mediated toxicity. The reduction of HTT protein and/or mRNA as well as HTT mediated toxicity may be accomplished with almost no enhanced inflammation.

In some embodiments, the AAV particle may be administered to a subject (e.g., to the CNS of a subject) in a therapeutically effective amount to slow the functional decline of a subject (e.g., determined using a known evaluation method such as the unified Huntington's disease rating scale (UHDRS)). As a non-limiting example, the vector may be administered via intraparenchymal injection.

In some embodiments, the AAV particle may be administered to the cisterna magna in a therapeutically effective amount to transduce medium spiny neurons, cortical neurons and/or astrocytes. As a non-limiting example, the vector may be administered intrathecally.

In some embodiments, the AAV particle may be administered using intrathecal infusion in a therapeutically effective amount to transduce medium spiny neurons, cortical neurons and/or astrocytes. As a non-limiting example, the vector may be administered intrathecally.

In some embodiments, the AAV particle comprising a modulatory polynucleotide may be formulated. As a non-limiting example, the baricity and/or osmolality of the formulation may he optimized to ensure optimal drug distribution in the central nervous system or a region or component of the central nervous system.

In some embodiments, the AAV particle comprising a modulatory polynucleotide may be delivered to a subject via a single route of administration.

In some embodiments, the AAV particle comprising a modulatory polynucleotide may be delivered to a subject via a multi-site route of administration. A subject may be administered the AAV particle comprising a modulatory polynucleotide at 2, 3, 4, 5 or more than 5 sites.

In some embodiments, a subject may be administered the AAV particle comprising a modulatory polynucleotide described herein using a bolus injection.

In some embodiments, a subject may be administered the AAV particle comprising a modulatory polynucleotide described herein using sustained delivery over a period of minutes, hours or days. The infusion rate may be changed depending on the subject, distribution, formulation or another delivery parameter.

In some embodiments, the AV particle described herein is administered via putamen and caudate infusion. As a non-limiting example, the dual infusion provides a broad striatal distribution as well as a frontal and temporal cortical distribution.

In some embodiments, the AAV particle is AAV-DJ8 which is administered via unilateral putamen infusion. As a non-limiting, example, the distribution of the administered AAV-DJ8 is similar to the distribution of AAV1 delivered via unilateral putamen infusion.

In some embodiments, the AAV particle described herein is administered via intrathecal (IT) infusion at C1. The infusion may be for 1, 2, 3, 4, 6, 7, 8, 9, 10, 11, 12, 13, 14, 15 or more than 15 hours.

In some embodiments, the selection of subjects for administration of the AAV particle described herein and/or the effectiveness of the dose, route of administration and/or volume of administration may be evaluated using imaging of the perivascular spaces (PVS) which are also known as Virchow-Robin spaces. PVS surround the arterioles and venules as they perforate brain parenchyma and are filled with cerebrospinal fluid (CSF)/interstitial fluid. PVS are common in the midbrain, basal ganglia, and centrum semiovale. While not wishing to be bound by theory, PVS may play a role in the normal clearance of metabolites and have been associated with worse cognition and several disease states including Parkinson's disease. PVS are usually are normal in size but they can increase in size in a number of disease states. Potter et al. (Cerebrovasc Dis. 2015 January; 39(4): 224-231; the contents of which are herein incorporated by reference in its entirety) developed a grading method where they studied a full range of PVS and rated basal ganglia, centrum semiovale and midbrain PVS. They used the frequency and range of PVS used by Mac and Lullich et al. (J Neurol Neurosurg Psychiatry. 2004 November; 75(11):1519-23; the contents of which are herein incorporated by reference in its entirety) and Potter et al. gave 5 ratings to basal ganglia and centrum semiovale PVS: 0 (none), 1 (1-10), 2 (11-20), 3 (21-40) and 4 (>40) and 2 ratings to midbrain PVS: 0 (non-visible) or 1 (visible). The user guide for the rating system by Potter et al. can be found at: www.sbirc.ed.acuk/documentslepvs-rating-scale-user-guide.pdf.

In some embodiments, AAV particles described herein is administered via thalamus infusion. Infusion into the thalamus may be bilateral or unilateral.

In some embodiments, AAV particles described herein are administered via putamen infusion. Infusion into the thalamus may be bilateral or unilateral.

In some embodiments. AAV particles described herein are administered via putamen and thalamus infusion. Dual infusion into the putamen and thalamus may maximize brain distribution via axonal transport to cortical areas. Evers et at. observed positive transduction of neurons in the motor cortex and part of the parietal cortex after bilateral injections of AAV5-GFP into the putamen and thalamus of tgHD minipigs (Molecular Therapy (2018), doi: 10.101.6/j.ymthe.2018.06.021). Infusion into the putamen and thalamus may be independently bilateral or unilateral. As a non-limiting example, AAV particles may be infused into the putamen and thalamus from both sides of the brain. As another non-limiting example, AAV particles may be infused into the left putamen and left thalamus, or right putamen and right thalamus. As yet another non-limiting example, AAV particles may be infused into the left putamen and right thalamus, or right putamen and left thalamus. Dual infusion may occur consecutively or simultaneously.

In some embodiments, the AAV particle comprising a modulatory polynucleotide may be delivered to a subject in the absence of gene therapy-related changes in body weight.

In some embodiments, the AAV particle comprising a modulatory polynucleotide may he delivered to a subject in the absence of gene therapy-related clinical signs, including but not limited to incoordination, inappetence, decreased feeding, and overall weakness.

In some embodiments, the AAV particle comprising a modulatory polynucleotide may be delivered to a subject in the absence of gene therapy-related changes to blood of a subject. In certain embodiments, the changes in blood of a subject are serum chemistry, and coagulation parameters.

In some embodiments, the AAV particle comprising a modulatory polynucleotide may he delivered to a subject in the absence of pathological changes to a tissue of a subject (e.g., brain of the subject). In certain embodiments the pathological change is a gross pathological change, such as, but not limited to, atrophy. In certain embodiments, the pathological change is a histopathological change, including but not limited to, HTT inclusions.

### Dosing

The pharmaceutical compositions of the present disclosure may be administered to a subject using any amount effective for reducing, preventing and/or treating a HTT associated disorder (e.g., Huntington' Disease (HD)), The exact amount required will vary from subject to subject, depending on the species, age, and general condition of the subject, the severity of the disease, the particular composition, its mode of administration, its mode of activity, and the like.

The compositions of the present disclosure are typically formulated in unit dosage form for ease of administration and uniformity of dosage. It will be understood, however, that the total daily usage of the compositions of the present disclosure may be decided by the attending physician within the scope of sound medical judgment. The specific therapeutic effectiveness for any particular patient will depend upon a variety of factors including the disorder being treated and the severity of the disorder: the activity of the specific compound employed; the specific composition employed; the age, body weight, general health, sex and diet of the patient; the time of administration, route of administration, and rate of excretion of the siRNA duplexes employed, the duration of the treatment; drugs used in combination or coincidental with the specific compound employed; and like factors well known in the medical arts.

In some embodiments, the age and sex of a subject may be used to determine the dose of the compositions of the present disclosure. As a non-limiting example, a subject who is older may receive a larger dose (e.g., 5-10%, 10-20%, 15-30%, 20-50%, 25-50% or at least 1%, 2%, 3%, 4%, 5%, 10%, 20%, 30%, 40%, 50%, 60%, 70%, 80%, 90% or more than 90% more) of the composition as compared to a younger subject. As another non-limiting example, a subject who is younger may receive a larger dose (e.g., 5-10%, 10-20%, 15-30%, 20-50%, 25-50% or at least 1%, 2%, 3%, 4%, 5%, 10%, 20%, 30%, 40%, 50%, 60%, 70%, 80%, 90% or more than 90% more) of the composition as compared to an older subject. As yet another non-limiting example, a subject who is female may receive a larger dose (e.g., 5-10%, 10-20%, 15-30%, 20-50%, 25-50% or at least 1%, 2%, 3%, 4%, 5%, 10%, 20%, 30%, 40%, 50%, 60%, 70%, 80%, 90% or more than 90% more) of the composition as compared to a male subject. As yet another non-limiting example, a subject who is male may receive a larger dose (e.g., 5-10%, 10-20%, 15-30%, 20-50%, 25-50% or at least 1%, 2%, 3%, 4%, 5%, 10%, 20%, 30%, 40%, 50%, 60%, 70%, 80%, 90% or more than 90% more) of the composition as compared to a female subject

In some specific embodiments, the doses of AAV particles the delivering siRNA duplexes of the present disclosure may be adapted depending on the disease condition, the subject and the treatment strategy.

In some embodiments, delivery of the compositions in accordance with the present disclosure to cells comprises a rate of delivery defined by [VG/hour=mL/hour*VG/mL] wherein VG is viral genomes, VG/mL is composition concentration, and mL/hour is rate of prolonged delivery.

In some embodiments, delivery of compositions in accordance with the present disclosure to cells may comprise a total concentration per subject between about 1×10⁶ VG and about 1×10⁶ VG. In some embodiments, delivery may comprise a composition concentration of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 1.1×10¹¹, 1.2×10¹¹, 1.3×10¹¹, 1.4×10¹¹, 1.5×10¹¹, 1.6×10¹¹, 1.7×10¹¹, 1.8×10¹¹, 1.9×10¹¹, 2×10¹¹, 2.1×10¹¹, 2.2×10¹¹, 2.3×10¹¹, 2.4×10¹¹, 2.5×10¹¹, 2.6×10¹¹, 2.7×10¹¹, 2.8×10¹¹, 2.9×10¹¹, 3×10¹¹, 4×10¹¹, 5×10¹¹, 6×10¹¹, 7×10¹¹, 7.1×10¹¹, 7.2×10¹¹, 7.3×10¹¹, 7.4×10¹¹, 7.5×10¹¹, 7.6×10¹¹, 7.7×10¹¹, 7.8×10¹¹, 7.9×10¹¹, 8×10¹¹, 9×10¹¹, 1×10¹², 1.1×10¹², 1.2×10¹², 1.3×10¹², 1.4×10¹², 1.5×10¹², 1.6×10¹², 1.7×10¹², 1.8×10¹², 1.9×10¹², 2×10¹², 2.1×10¹², 2.2×10¹², 2.3×10¹², 2.4×10¹², 2.5×10¹², 2.6×10¹², 2.7×10¹², 2.8×10¹², 2.9×10¹², 3×10¹², 3.1×10¹², 3.2×10¹², 3.3×10¹², 3.4×10¹², 3.5×10¹², 3.6×10¹², 3.7×10¹², 3.8×10¹², 3.9×10¹², 4×10¹², 4.1×10¹², 4.2×10¹², 4.3×10¹², 4.4×10¹², 4.5×10¹², 4.6×10¹², 4.7×10¹², 4.8×10¹², 4.9×10¹², 5×10¹², 6×10¹², 6.1×10¹², 6.2×10¹², 6.3×10¹², 6.4×10¹², 6.5×10¹², 6.6×10¹², 6.7×10¹², 6.8×10¹², 6.9×10¹², 7×10¹², 8×10¹², 8.1×10¹², 8.2×10¹², 8.3×10¹², 8.4×10¹², 8.5×10¹², 8.6×10¹², 8.7×10¹², 8.8×10¹², 8.9×10¹², 9×10¹², 1×10¹³, 1.1×10¹³, 1.2×10¹³, 1.3×10¹³, 1.4×10¹³, 1.5×10¹³, 1.6×10¹³, 1.7×10¹³, 1.8×10¹³, 1.9×10¹³, 2×10¹³, 2.7×10¹³, 3×10¹³, 4×10¹³, 5×10¹³, 6×10¹³, 6.7×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵ 9×10¹⁵, or 1×10¹⁶ VG/subject or VG/dose.

In some embodiments, delivery of compositions in accordance with the present disclosure to cells may comprise a total concentration per subject between about 1×10⁶ VG/kg and about 1×10¹⁶ VG/kg. In some embodiments, delivery may comprise a composition concentration of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁴, 4×10¹⁰, 5×10¹⁰, 6×10¹⁰, 7×10¹⁰, 8×10¹⁰, 9×10¹⁰, 1×10¹¹, 1.1×10¹¹, 1.2×10¹¹, 1.3×10¹¹, 1.4×10¹¹, 1.5×10¹¹, 1.6×10¹¹, 1.7×10¹¹, 1.8×10¹¹, 1.9×10¹¹, 2×10¹¹, 2.1×10¹¹, 2.2×10¹¹, 2.3×10¹¹, 2.4×10¹¹, 2.5×10¹¹, 2.6×10¹¹, 2.7×10¹¹, 2.8×10¹¹, 2.9×10¹¹, 3×10¹¹, 4×10¹¹, 5×10¹¹, 6×10¹¹, 7×10¹¹, 7.1×10¹¹, 7.2×10¹¹, 7.3×10¹¹, 7.4×10¹¹, 7.5×10¹¹, 7.6×10¹¹, 7.7×10¹¹, 7.8×10¹¹, 7.9×10¹¹, 8×10¹¹, 9×10¹¹, 1×10¹², 1.1×10¹², 1.2×10¹², 1.3×10¹², 1.4×10¹², 1.5×10¹², 1.6×10¹², 1.7×10¹², 1.8×10⁷ 1.9×10¹², 2×10¹², 2.1×10^{.12} 2.2×10¹², 2.3×10¹², 2.4×10¹², 2.5×10¹², 2.6×10¹², 2.7×10¹², 2.8×10¹², 2.9×10¹², 3×10¹², 3.1×10¹² 3.2×10¹², 3.3×10¹² 3.4×10¹², 3.5×10¹², 3.6×10¹², 3.7×10¹², 3.8×10¹², 3.9×10¹², 4×10¹², 4.1×10¹², 4.2×10¹², 4.3×10¹², 4.4×10¹², 4.5×10¹², 4.6×10¹², 4.7×10¹², 4.8×10¹², 4.9×10¹², 5×10¹², 6×10¹², 6.1×10¹², 6.2×10¹², 6.3×10¹², 6.4×10¹², 6.5×10¹², 6.6×10¹², 6.7×10¹², 6.8×10¹², 6.9×10¹², 7×10¹², 8×10¹², 8.1×10¹², 8.2×10¹², 8.3×10¹², 8.4×10¹², 8.5×10¹², 8.6×10¹², 8.7×10¹², 8.8×10¹², 8.9×10¹², 9×10¹², 1×10¹³, 1.1×10¹³, 1.2×10¹³, 1.3×10¹³, 1.4×10¹³, 1.5×10¹³, 1.6×10¹³, 1.7×10¹³, 1.8×10¹³, 1.9×10¹³, 2×10¹³, 2.7×10¹³, 3×10¹³, 4×10¹³, 5×10¹³, 6×10¹³, 6.7×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 78×10¹⁵, 9×10¹⁵, or 1×10¹⁶ VG/kg.

In some embodiments, delivery of the compositions to accordance with be present disclosure to cells may comprise a total concentration between about 1×10⁶ VG/mL and about 1×10¹⁶ VG/mL. In some embodiments, delivery may comprise a composition concentration of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 1.1×10¹¹, 1.2×10¹¹, 1.3×10¹¹, 1.4×10¹¹, 1.5×10¹¹, 1.6×10¹¹, 1.7×10¹¹, 1.8×10¹¹, 1.9×10¹¹, 2×10¹¹, 3×10¹¹, 4×10¹¹, 5×10¹¹, 6×10¹¹, 7×10¹¹, 8×10¹¹, 9×10¹¹, 1×10¹², 1.1×10¹², 1.2×10¹², 1.3×10¹², 1.4×10¹², 1.5×10¹², 1.6×10¹², 1.7×10¹², 1.8×10¹², 1.9×10¹², 2×10¹², 2.1×10¹² 2.2×10¹², 3×10¹², 2.4×10¹², 2.5×10¹², 2.6×10¹², 2.7×10¹², 2.8×10¹², 2.9×10¹², 3×10¹², 3.1×10¹², 3.2×10¹², 3.3×10¹², 3.4×10¹², 3.5×10¹², 3.6×10¹², 3.7×10¹², 3.8×10¹², 3.9×10 4×10¹², 4.1×10¹², 4.2×10¹², 4.3×10¹², 4.4×10³², 4.5×10¹², 4.6×10¹², 4.7×10¹², 4.8×10¹², 49×10¹², 5×10¹², 6×10¹², 6.1×10¹², 6.2×10¹², 6.3×10¹², 6.4×10¹², 6.5×10¹², 6.6×10¹², 6.7×10¹², 6.8×10¹², 6.9×10¹², 7×10¹², 8×10¹², 9×10¹², 1×10¹³, 1.1×10¹³, 1.2×10¹³, 1.3×10¹³, 1.4×10¹³, 1.5×10¹³, 1.6×10¹³, 1.7×10¹³, 1.8×10¹³, 1.9×10¹³, 2×10¹³, 2.7×10¹³, 3×10¹³, 4×10¹³, 5×10¹³, 6×10¹³, 6.7×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴⁹, 8×10¹⁴, 9×10¹⁴, 1×10¹⁵, 2×10¹⁵, 3×10¹⁵, 4×10¹⁵, 5×10¹⁵, 6X10¹⁵, 7×10¹⁵, 8×10¹⁵, 9×10¹⁵, or 1×10¹⁶ VG/mL.

In some embodiments, the compositions in accordance with the present disclosure to be delivered may comprise a concentration between 9×10¹¹VG/mL-2.7×10¹³ VG/mL. In some embodiments, the compositions in accordance with the present disclosure to be delivered may comprise a concentration of 2.7×10¹³ VG/mL.

In some embodiments, delivery of the compositions in accordance with the present disclosure o cells may comprise a total concentration between about 1×10⁶ total capsid/mL and about 1×10¹⁶ total capsid/mL. In some embodiments, delivery may comprise a composition concentration of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 1.1×10¹², 1.2×10¹², 1.3×10¹², 4×10¹², 1.5×10¹², 1.6×10¹², 1.7×10¹², 1.8×10¹², 1.9×10¹², 2×10¹², 2.1×10¹², 2.2×10¹², 2.3×10¹², 2.4×10¹², 2.5×10¹², 2.6×10¹², 2.7×10¹², 2.8×10¹², 2.9×10¹²², 3×10¹², 3.1×10¹², 3.2×10¹² 3.3×10¹², 3.4×10¹², 3.5×10¹², 3.6×10¹², 3.7×10¹², 3.8×10¹², 3.9×10¹², 4×10¹², 4.1×10¹², 2×10¹², 4.3×10¹², 4.4×10¹², 4.5×10¹², 4.6×10¹², 4.7×10¹², 4.8×10¹², 4.9×10¹², 5×10¹², 6×10¹², 7×10¹², 8×10¹², 9×10¹², 1×10¹³, 2×10¹³, 2.7×10¹³, 3×10¹³, 4×10¹³, 5×10¹³, 6×10¹³, 6.7×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ total capsid/mL.

In certain embodiments, the desired siRNA duplex dosage may be delivered using multiple administrations (e.g., two, three, tour, five, six, seven, eight, nine, ten, eleven, twelve, thirteen, fourteen, or more administrations). When multiple administrations are employed, split dosing regimens such as those described herein may be used. As used herein, a “split dose” is the division of single unit dose or total daily dose into two or more doses, e.g., two or more administrations of the single unit dose. As used herein, a “single unit dose” is a dose of any modulatory polynucleotide therapeutic administered in one dose/at one time/single route/single point of contact, i.e., single administration event. As used herein, a “total daily dose” is an amount given or prescribed in a 24-hour period. It may be administered as a single unit dose. In some embodiments, the AAV particles comprising the modulatory polynucleotides of the present disclosure are administered to a subject in split doses. They may be formulated in buffer only or in a formulation described herein.

In some embodiments, the dose, concentration and/or volume of the composition described herein may be adjusted depending on the contribution of the caudate or putamen to cortical and subcortical distribution after administration. The administration may be intracerebroventricular, intrastriatal, intraputaminal, intrathalamic, intraparenchymal, subpial. and/or intrathecal administration.

In some embodiments, the dose, concentration and/or volume of the composition described herein may be adjusted depending on the cortical and neuraxial distribution following administration by intracerebroventricular, intrastriatal, intraputaminal, intrathalamic, intraparenchymal, subpial, and/or intrathecal delivery.

The volume of the pharmaceutical compositions to be administered may be determined based on the subject, the volume of the targeted structure, and/or the dose of the composition. In some embodiments, the subject is a rodent such as, but not limited to a mouse or a rat. The mouse or the rat may be a wild-type (WT) or a transgenic rat or mouse. As a non-limiting example, the transgenic mouse is the YAC128 mouse. As another non-limiting example, the transgenic mouse is a BACHD mouse. In some embodiments, the subject is a primate. In some embodiments, the subject is a non-human primate. In some embodiments, the subject is a human.

In some embodiments, the volume of the pharmaceutical composition to be infused to a putamen or thalamus in a subject may be between about 0.5-3000 μL per side. In some embodiments, the volume of the composition to be infused to a putamen or thalamus may be about 5 μl, 10 μl, 25 μl, 50 μl, 75 μl, 100 μl, 125 μl, 150 μl, 175 μl, 200 μl, 225 μl, 250 μl, 275 μl, 300 μl, 325 μl, 350 μl, 375 μl, 400 μl, 425 μl, 450 μl, 475 μl, 500 525 μl, 550 μl, 575 μl, 600 μl, 625 μl, 650 μl, 675 μl, 700 μl, 725 μl, 750 μl, 775 μl, 800 μl, 825 μl, 850 μl, 875 μl, 900 μl, 925 μl, 950 μl, 975 μl, 1000 μl, 1025 μl, 1050 μl, 1075 μl, 1100 μl, 1125 μl, 1150 μl, 1175 μl, 1200 μl, 1225 μl, 1250 μl, 1275 μl, 1300 μl, 1325 μl, 1350 μl, 1375 μl, 1400 μl, 1425 μl, 1450 μl, 1475 μl, 1500 μl, 1600 μl, 1700 μl, 1800 μl, 1900 μl, 2000 μl, 2250 μl, 2500 μl, 2750 μl, or 3000 μl per side.

In some embodiments, the volume of the pharmaceutical composition to be infused to a striatum in a subject may be between about 5-3000 μL per side. In some embodiments, the volume of the composition to be infused to a putamen or thalamus may be about 5 10 μl, 25 μl, 50 μl, 75 μl, 100 μl, 125 μl, 150 μl, 175 μl, 200 μl, 225 μl, 250 μl, 275 μl, 300 μl, 325 μl, 350 μl, 375 μl, 400 μl, 425 μl, 450 μl, 475 μl, 500 μl, 525 μl, 550 μl, 575 μl, 600 μl, 625 μl, 650 μl, 675 μl, 700 μl, 725 μl, 750 μl, 775 μl, 800 μl, 825 850 μl, 875 μl, 900 μl, 925 μl, 950 μl, 975 μl, 1000 μl, 1025 μl, 1050 μl, 1075 μl, 1100 μl, 1125 μl, 1150 μl, 1175 μl, 1200 μl, 1225 μl, 1250 μl, 1275 μl, 1300 μl, 1325 μl, 1350 μl, 1375 μl, 1400 μl, 1425 μl, 1450 μl, 1475 μl, 1500 μl, 1600 μl, 1700 μl, 1800 μl, 1900 μl, 2000 μl, 2250 μl, 2500 μl, 2750 μl, or 3000 μl, per side.

In some embodiments, the pharmaceutical composition described herein is administered to a subject which is mouse. In some embodiments, the volume of the composition to be infused to the striatum of the mouse is 1-10 μl, per side. In some embodiments, the volume of the composition to be infused to the striatum in a mouse may be about 1 μl, 2 μl, 3 μl, 4 μl, 5 μl, 6 μl, 7 μl, 8 μl, 9 μl, or 10 μl per side. In some embodiments, the volume of the composition to be infused to the striatum in a mouse is 5 μl per side.

In some embodiments, the pharmaceutical composition described herein is administered to a subject which is a non-human primate. In some embodiments, the volume of the composition to be infused to the putamen in a non-human primate is 50-150 μl per side. In some embodiments, the volume of the composition to be infused to the putamen in a non-human primate is 100-200 μl per side. in some embodiments, the volume of the composition to he infused to the putamen in a non-human primate is 175-525 μL per side.

In some embodiments, the volume of the composition to be infused to the thalamus in a non-human primate is 70-250 μL per side. In some embodiments, the volume of the composition to be infused to the thalamus in a non-human primate is 100-250 μl, per side. In some embodiments, the volume of the composition to be infused to the thalamus in a non-human is 200-300 μL per side. In some embodiments, the volume of the composition to be mused to the thalamus in a non-human primate is 450-1500 μL per side.

In some embodiments, the pharmaceutical composition described herein is administered to a subject Which is a human. In some embodiments, the volume of the pharmaceutical composition administered to the putamen in a human may be no more than 2000 μL/hemisphere. In some embodiments, the volume of the composition to be infused to the putamen in a human is no more than 1500 μL/hemisphere per side.

In some embodiments, the volume of the composition to be infused to the putamen in a human is 300-1500 μL per side. In some embodiments, the volume of the composition to be infused to the putamen in a human may be about 300 μl, 325 μl, 350 μl, 375 μl, 400 μl, 425 μl, 450 μl, 475 μl, 500 μl, 525 μl, 550 μl, 575 μl, 600 μl, 625 μl, 650 μl, 675 μl, 700 μl, 725 μl, 750 μl, 775 μl, 800 μl, 825 μl, 850 μl, 875 μl, 900 μl, 925 μl, 950 μl, 975 μl, 1000 μl, 1025 μl, 1050 μl, 1075 μl, 1100 μl, 1125 μl, 1150 μl, 1175 μl, 1200 μl, 1225 μl, 1250 μl, 1275 μl, 1300 μl, 1325 μl, 1350 μl, 1375 μl, 1400 μl, 1425 μl, 1450 μl, 1475 μl, or 1500 μl per side. In some embodiments, the volume of the composition to be infused to the putamen in a human is 900 μl per side.

In some embodiments, the volume of the pharmaceutical composition administered to the thalamus in a human may be no more than 3000 μL/hemisphere, In some embodiments, the volume of the composition to be infused to a thalamus in a human is no more than 2500 μl, per side.

In some embodiments, the volume of the composition to be infused to a thalamus in a human is 1300-2500 μl, per side. In some embodiments, the volume of the composition to be infused to a thalamus in a human may be 1300 μL, 1325 μL, 1350 μL, 1375 μL, 1400 μL, 1425 μL, 1450 μL, 1475 μL, 1500 μL, 1525 μL, 1550 μL, 1575 μL, 1600 μL, 1625 μL, 1650 μL, 1675 μL, 1700 μL, 1725 μL, 1750 μL, 1775 μL, 1800 μL, 1825 μL, 1850 μL, 1875 μL, 1900 μL, 1925 μL, 1950 μL, 1975 μL, 2000 μL, 2025 μL, 2050 μL, 2075 μL, 2100 μL, 2125 μL, 2150 μL, 2175 μL, 2200 μL, 2225 μL, 2250 μL, 2275 μL, 2300 μL, 2325 μL, 2350 μL, 2375 μL, 2400 μL, 2425 μL, 2450 μL, 2475 μL, or 2500 μL per side. In some embodiments, the volume of the composition to be infused to the thalamus in a human is 1700 μl per side.

In some embodiments, the dose administered to the striatum in a subject may be about 1×10⁹ to 1×10¹⁵ VG per side. In some embodiments, the dose administered to the striatum in a subject may be about 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰ 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1.5×10¹², 2×10¹², 2.5×10¹², 3×10¹², 3.5×10¹², 4×10¹², 4.5×10¹², 5×10¹², 5.5×10¹², 6×10¹², 6.5×10¹², 7×10¹², 7.5×10¹², 8×10¹², 8.5×10¹², 9×10¹², 9.5×10¹², 1×10¹³, 1.5×10¹³, 2×10¹³, 2.5×10¹³, 3×10¹³, 3.5×10¹³, 4×10¹³, 4.5×10¹³, 5×10¹³, 5.5×10¹³, 6×10¹³, 6.5×10¹³, 7×10¹³, 7.5×10¹³, 8×10¹³, 8.5×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, or 1×10¹⁵ VG per side.

In some embodiments, the dose administered to the putamen in a subject may be about 1×10¹⁰ to 1×10¹⁵ VG per side. In some embodiments, the dose administered to the putamen in a subject may be about 1×10¹⁰, 5×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 1.5×10¹², 2×10¹², 2.5×10¹², 3×10¹², 3.5×10¹², 4×10¹², 4.5×10¹², 5×10¹², 5.5×10¹², 6×10¹², 6.5×10¹², 7×10¹², 7.5×10¹², 8×10¹², 8.5×10¹², 9×10¹², 9.5×10¹², 1×10¹³, 1.5×10¹³, 2×10¹³, 2.5×10¹³, 3×10¹³, 3.5×10¹³, 4×10¹³, 4.5×10¹³, 5×10¹³, 5.5×10¹³, 6×10¹³, 6.5×10¹³, 7×10¹³, 7.5×10¹³, 8×10¹³, 8.5×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, or 1×10¹⁵ VG per side.

In some embodiments, the dose administered to the thalamus in a subject may be about 1×10¹⁰to 1×10¹⁵ VG per side. In some embodiments, the close administered to the thalamus in a subject may be about 1×10¹⁰, 5×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 1.5×10¹², 2×10¹², 2.5×10¹², 3×10¹², 3.5×10¹², 4×10¹², 4.5×10¹², 5×10¹², 5.5×10¹², 6×10¹², 6.5×10¹², 7×10¹², 7.5×10¹², 8×10¹², 8.5×10¹², 9×10¹², 9.5×10¹², 1×10¹³, 1.5×10¹³, 2×10¹³, 2.5×10¹³, 3×10¹³, 3.5×10¹³, 4×10¹³, 4.5×10¹³, 5×10¹³, 5.5×10¹³, 6×10¹³, 6.5×10¹³, 7×10¹³, 7.5×10¹³, 8×10¹³, 8.5×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, or 1×10¹⁵ VG per side.

In some embodiments, the total dose administered to the striatum in a subject may be about 1×10⁹ to 5×10¹⁵ VG. In some embodiments, the dose administered to the striatum in a subject may be about 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 1.5×10¹², 2×10¹², 2.5×10¹², 3×10¹², 3.5×10¹², 4×10¹², 4.5×10¹², 5×10¹², 5.5×10¹², 6×10¹², 6.5×10¹², 7×10¹², 7.5×10¹², 8×10¹², 8.5×10¹², 9×10¹². 9.5×10¹², 1×10¹³, 1.5×10¹³, 2×10¹³, 2.5×10¹³, 3×10¹³, 3.5×10¹³, 4×10¹³, 4.5×10¹³, 5×10¹³, 5.5×10¹³, 6×10¹³, 6.5×10¹³, 7×10¹³, 7.5×10¹³, 8×10¹³, 8.5×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, or 1×10¹⁵ 2×10¹⁵, 3×10¹⁵, 4×10¹⁵, or 5×10¹⁵ VG.

In some embodiments, the total dose administered to the subject via putamen and thalamus infusion is 1×10¹⁰to 5×10¹⁵ VG. In some embodiments, the total dose administered to the subject via putamen and thalamus infusion may be about 1×10¹⁰, 5×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 1.5×10¹², 2×10¹², 2.5×10¹², 3×10¹², 3.5×10¹², 4×10¹², 4.5×10¹², 5×10¹², 5.5×10¹², 6×10¹², 6.5×10¹², 7×10¹², 7.5×10¹², 8×10¹², 8.5×10¹², 9×10¹², 9.5×10¹², 1×10¹³, 1.5×10¹³, 2×10¹³, 2.5×10¹³, 3×10¹³, 3.5×10¹³, 4×10¹³, 4.5×10¹³, 5×10¹³, 5.5×10¹³, 6×10¹³, 6.5×10¹³, 7×10¹³, 7.5×10¹³, 8×10¹³, 8.5×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, or 5×10¹⁵ VG.

In some embodiments, the dose administered to the striatum in mouse may be about 4×10⁹ to 2×10¹¹ VG per side. In some embodiments, the dose administered to the striatum in a mouse may be about 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹ or 2×10¹¹ VG per side. In some embodiments, the dose administered to the striatum in a mouse may be 4.4×10⁹ VG per side. In some embodiments, the dose administered to the striatum in a mouse may be 1.4×10¹⁰ VG per side. In some embodiments, the dose administered to the striatum in a mouse may be 4.4×10¹⁰ VG per side. In some embodiments, the dose administered to the striatum in a mouse may be 1.4×10¹¹ VG per side.

In some embodiments, the dose administered to the putamen in a non-human primate may be about 9×10¹⁰ to 5.5×10¹² VG per side. In some embodiments, the dose administered to the putamen in a non-human primate may be about 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 1.5×10¹², 2×10¹², 2.5×10¹², 3×10¹², 3.5×10¹², 4×10¹², 4.5×10¹², 5×10¹², or 5.5×10¹² VG per side.

In some embodiments, the dose administered to the putamen in a non-human primate may be about 2×10¹⁰ to 5×10¹¹ VG per side. In some embodiments, the dose administered to the putamen in a non-human primate may be about 2.0×10¹⁰, 2.2×10¹⁰, 2.4×10¹⁰, 2.6×10¹⁰, 2.8×10¹⁰, 3.0×10¹⁰, 3.2×10¹⁰, 3.4×10¹⁰, 3.6×10¹⁰, 3.8×10¹⁰, 4.0×10¹⁰, 4.2×10¹⁰, 4.4×10¹⁰, 4.6×10¹⁰, 4.8×10¹⁰, 5.0×10¹⁰, 5.2×10¹⁰, 5.4×10¹⁰, 5.6×10¹⁰, 5.8×10¹⁰, 6.0×10¹⁰, 6.2×10¹⁰, 6.4×10¹⁰, 6.6×10¹⁰, 6.8×10¹⁰, 7.0×10¹⁰, 7.2×10¹⁰, 7.4×10¹⁰, 7.6×10¹⁰, 7.8×10¹⁰, 8.0×10¹⁰, 8.2×10¹⁰, 8.4×10¹⁰, 8.6×10¹⁰, 8.8×10¹⁰, 9.0×10¹⁰, 9.2×10¹⁰, 9.4×10¹⁰, 9.6×10¹⁰, 9.8×10¹⁰, 1.0×10¹¹, 1.2×10¹¹, 1.4×10¹¹, 1.6×10¹¹, 1.8×10¹¹, 2.0×10¹¹, 2.2×10¹¹, 2.4×10¹¹, 2.6×10¹¹, 2.8×10¹¹, 3.0×10¹¹, 3.2×10¹¹, 3.4×10¹¹, 3.6×10¹¹, 3.8×10¹¹, 4.0×10¹¹, 4.2×10¹¹, 4.4×10¹¹, 4.6×10¹¹, 4.8×10¹¹, 5.0×10¹¹VG per side.

In some embodiments, the dose administered to the thalamus in a non-human primate may be about 1.5×10¹¹ to 8.5×10¹² VG per side. in some embodiments, the dose administered to the thalamus in a non-human primate may be about 1.5×10¹¹, 1.8×10¹¹, 2×10¹¹, 3×10¹¹, 4×10¹¹, 5×10¹¹, 6×10¹¹, 7×10¹¹, 8×10¹¹, 9×10¹¹, 1×10¹², 1.5×10¹², 2×10¹², 2.5×10¹², 3×10¹², 3.5×10¹², 4×10¹², 4.5×10¹², 5×10¹², 5.5×10¹², 6×10¹², 6.5×10¹², 7×10¹², 7.5×10¹², 8×10¹², or 8.5×10¹² VG per side.

In some embodiments, the dose administered to the thalamus a non-human primate may be about 4.0×10¹⁰ to 7.0×10¹¹ VG per side. In some embodiments, the dose administered to the thalamus in a non-human primate may be about 4.0×10¹⁰, 4.2×10¹⁰, 4.4×10¹⁰, 4.6×10¹⁰, 4.8×10¹⁰, 5.0×10¹⁰, 5.2×10¹⁰, 5.4×10¹⁰, 5.6×10¹⁰, 5.8×10¹⁰, 6.0×10¹⁰, 6.2×10¹⁰, 6.4×10¹⁰, 6.6×10¹⁰, 6.8×10¹⁰, 7.0×10¹⁰, 7.2×10¹⁰, 7.4×10¹⁰, 7.6×10¹⁰, 7.8×10¹⁰, 8.0×10¹⁰, 8.2×10¹⁰, 8.4×10¹⁰, 8.6×10¹⁰, 8.8×10¹⁰, 9.0×10¹⁰, 9.2×10¹⁰, 9.4×10¹⁰, 9.6×10¹⁰, 9.8×10¹⁰, 1.0×10¹¹, 1.2×10¹¹, 1.4×10¹¹, 1.6×10¹¹, 1.8×10¹¹, 2.0×10¹¹, 2.2×10¹¹, 2.4×10¹¹, 2.6×10¹¹, 2.8×10¹¹, 3.0×10¹¹, 3.2×10¹¹, 3.4×10¹¹, 3.6×10¹¹, 3.8×10¹¹, 4.0×10¹¹, 4.2×10¹¹, 4.4×10¹¹, 4.6×10¹¹, 4.8×10¹¹, 5.0×10¹¹, 5.2×10¹¹, 5.4×10¹¹, 5.6×10¹¹, 5.8×10¹¹, 6.0×10¹¹, 6.2×10¹¹, 6.4×10¹¹, 6.6×10¹¹, 6.8×10¹¹, or 7.0×10¹¹VG per side.

In some embodiments, the total dose administered to the mouse via striatum infusion is 8×10⁹to 3×10¹¹ VG. In some embodiments, the total dose administered to the mouse via striatum infusion may be about 8.0×10¹⁰, 8.2×10¹⁰, 8.4×10¹⁰, 8.6×10¹⁰, 8.8×10¹⁰, 9.0×10¹⁰, 9.2×10¹⁰, 9.4×10¹⁰, 9.6×10¹⁰, 9.8×10¹⁰, 1.0×10¹¹, 1.2×10¹¹, 1.4×10¹¹, 1.6×10 ¹¹, 1.8×10¹¹, 2.0×10¹¹, 2.2×10¹¹, 2.4×10¹¹, 2.6×10¹¹, 2.8×10¹¹, 3.0×10¹¹ VG. In some embodiments, the total dose administered to the mouse via striatum infusion is 8.8×10⁹ VG. In some embodiments, the total dose administered to the mouse via striatum infusion is 2.8×10¹⁰ VG. In some embodiments, the total dose administered to the mouse via striatum infusion is 8.8×10¹⁰ VG. In some embodiments, the total dose administered to the mouse via striatum infusion is 2.8×10¹¹ VG

In some embodiments, the total dose administered to the non-human primate via putamen and thalamus infusion is 5×10¹¹ to 3×10¹³ VG. In some embodiments, the total dose administered to the non-human primate via putamen and thalamus infusion may be about 5×10¹¹, 6×10¹¹, 7×10¹¹, 8×10¹¹, 9×10¹¹, 1×10¹², 1.5×10¹², 2×10¹², 2.5×10¹², 3×10¹², 3.5×10¹², 4×10¹², 4.5×10¹², 5×10¹², 5.5×10¹², 6×10¹², 6.5×10¹², 7×10¹², 7.5×10¹², 8×10¹², 8.5×10¹², 9×10¹², 9.5×10¹², 1×10¹³, 1.5×10¹³, 2×10¹³, 2.5×10¹³, or 3×10¹³ VG.

In some embodiments, the total dose administered to the non-human primate via putamen and thalamus infusion is 1×10¹¹to 2×10¹³ VG, In some embodiments, the total dose administered to the non-human primate via putamen and thalamus infusion may be about 1×10¹¹, 1.5×10¹¹, 2×10¹¹, 2.5×10¹¹, 3×10¹¹, 3.5×10¹¹, 4×10¹¹, 4.5×10¹¹, 5×10¹¹, 5.5×10¹¹, 6×10¹¹, 6.5×10¹¹, 7×10¹¹, 7.5×10¹¹, 8×10¹¹, 8.5×10¹¹, 9×10¹¹, 9.5×10¹¹, 1×10¹², 1.5×10¹², 2×10¹², 2.5×10¹², 3×10¹², 3.5×10¹², 4×10¹², 4.5×10¹², 5×10¹², 5.5×10¹², 6×10¹², 6.5×10¹², 7×10¹², 7.5×10¹², 8×10¹², 8.5×10¹², 9×10¹², 9.5×10¹², 1×10¹³, 1.5×10¹³, 2×10¹³ VG. In certain embodiments, the total dose administered to the non-human primate via putamen and thalamus infusion is 1.5×10¹¹ VG. In certain embodiments, the total dose administered to the non-human primate via putamen and thalamus infusion is 1.4×10¹¹ VG. In certain embodiments, the total dose administered to the non-human primate via putamen and thalamus infusion is 4.5×10¹¹ VG. In certain embodiments, the total dose administered to the non-human primate via putamen and thalamus infusion is 1.4×10¹² VG. In certain embodiments, the total dose administered to the non-human primate via putamen and thalamus infusion is 2.2×10¹² VG. In certain embodiments, the total dose administered to the non-human primate via putamen and thalamus infusion is 1.8×10¹³ VG.

In some embodiments, the dose administered to the putamen in a human may be about 2.5×10¹¹ to 4.5×10¹³ VG per side. In some embodiments, the dose administered to the putamen in a human may be about 2.5×10¹¹, 3×10¹¹, 4×10¹¹, 5×10¹¹, 6×10¹¹, 7×10¹¹, 8×10¹¹, 9×10¹¹, 1×10¹², 1.5×10¹², 2×10¹², 2.5×10¹², 3×10¹², 3.5×10¹², 4×10¹², 4.5×10¹², 5×10¹², 5.5×10¹², 6×10¹², 6.5×10¹², 7×10¹², 7.5×10¹², 8×10¹², 8.5×10¹², 9×10¹², 9.5×10¹², 1×10¹³, 1.5×10¹³, 2×10¹³, 2.5×10¹³, 3×10¹³, 3.5×10¹³, 4×10¹³, or 4.5×10¹³ VG per side. In some embodiments, the dose administered to the putamen in a human may be between 8×10¹¹ to 4×10¹³ VG per side.

In some embodiments, the dose administered to the thalamus in a human may be about 1×10¹² to 7×10¹³ VG per side. In some embodiments, the dose administered to the thalamus in a human may be about 1×10¹², 1.5×10¹², 2×10¹², 2.5×10¹², 3×10¹², 3.5×10¹², 4×10¹², 4.5×10¹², 5×10¹², 5.5×10¹², 6×10¹², 6.5×10¹², 7×10¹², 7.5×10¹², 8×10¹², 8.5×10¹², 9×10¹², 9.5×10¹², 1×10¹³, 1.5×10¹³, 2×10¹³, 2.5×10¹³, 3×10¹³, 3.5×10¹³, 4×10¹³, 4.5×10¹³, 5×10¹³, 5.5×10¹³, 6×10¹³, 6.5×10¹³, 6.8×10¹³, 7×10¹³ VG per side. In some embodiments, the close administered to the thalamus is between 3.5×10¹² to 6.8×10¹³ VG per side.

In some embodiments, the total dose administered to the human via putamen and thalamus infusion is 2.5×10¹² to 2.5×10¹⁴ VG. In some embodiments, the total dose administered to the human via putamen and thalamus infusion may be about 2.5×10¹², 3×10¹², 3.5×10¹², 4×10¹², 4.5×10¹², 5×10¹², 5.5×10¹², 6×10¹², 6.5×10¹², 7×10¹², 7.5×10¹², 8×10¹², 8.5×10¹², 8.6×10¹², 9×10¹², 9.5×10¹², 1×10¹³, 1.5×10¹³, 2×10¹³, 2.5×10¹³, 3×10¹³, 3.5×10¹³, 4×10¹³, 4.5×10¹³, 5×10¹³, 5.5×10¹³, 6×10¹³, 6.5×10¹³, 7×10¹³, 7.5×10¹³, 8×10¹³, 8.5×10¹³, 9×10¹³. 1×10¹⁴, 2×10¹⁴, 2.1×10¹⁴, 2.2×10¹⁴, 23×10¹⁴, 2.4×10¹⁴, or 2.5×10¹⁴ VG. In some embodiments, the total dose administered to the subject is between 8.6×10¹² to 2×10¹⁴ VG.

In some embodiments, dose volumes may be deposited into infusion site using ascending infusion rates. As a non-limiting example, dose volumes may be deposited into Infusion site in 3 different stages (e.g., at dose rates of 1, 3, 5 μL/min) with appropriate durations to complete the total dose volume.

In some embodiments, dose volumes mar be deposited into infusion site using a range of infusion rates. As a non-limiting example, dose volumes may be deposited into infusion site at dose rates ranging from 1 μL/min to 5 μL/min, with appropriate durations to complete the total dose volume.

In some embodiments, dose volumes may be deposited into infusion site using an infusion rate of 0.5 μL/min, with an appropriate duration to complete the total dose volume.

### IV. Kits and Devices

### Kits

In some embodiments, the disclosure provides a variety of kits for conveniently and/or effectively carrying out methods of the present disclosure. Typically, kits will comprise sufficient amounts and/or numbers of components to allow a user to perform multiple treatments of a subject(s) and/or to perform multiple experiments.

Any of the AAV particles of the present disclosure may be comprised in a kit. In some embodiments, kits may further include reagents and/or instructions for creating and/or synthesizing compounds and/or compositions of the present disclosure. In some embodiments, kits may also include one or more buffers. In some embodiments, kits of the disclosure may include components for making protein or nucleic acid arrays or libraries and thus, may include, for example, solid supports.

In some embodiments, kit components may be packaged either in aqueous media or in lyophilized form. The container means of the kits will generally include at least one vial, test tube, flask, bottle, syringe or other container means, into which a component may be placed, and preferably, suitably aliquoted. Where there is more than one kit component, (labeling reagent and label may be packaged together), kits may also generally contain second, third or other additional containers into which additional components may be separately placed. In some embodiments, kits may also comprise second container means for containing sterile, pharmaceutically acceptable buffers and/or other diluents. In some embodiments, various combinations of components may be comprised in one or more vial. Kits of the present disclosure may also typically include means for containing compounds and/or compositions of the present disclosure, e.g., proteins, nucleic acids, and any other reagent containers in close confinement for commercial sale. Such containers may include injection or blow-molded plastic containers into which desired vials are retained.

In some embodiments, kit components are provided in one and/or more liquid solutions. In some embodiments, liquid solutions are aqueous solutions, with sterile aqueous solutions being particularly preferred. In some embodiments, kit components may be provided as dried powder(s). When reagents and/or components are provided as dry powders, such powders may be reconstituted by the addition of suitable volumes of solvent. In some embodiments, it is envisioned that solvents may also be provided in another container means. In some embodiments, labeling dyes are provided as dried powders. In some embodiments, it is contemplated that 10, 20, 30, 40, 50, 60, 70, 80, 90, 100, 120, 120, 130, 140, 150, 160, 170, 180, 190, 200, 300, 400, 500, 600, 700, 800, 900, 1000 micrograms or at least or at most those amounts of dried dye are provided in kits of the disclosure. In such embodiments, dye may then he resuspended in any suitable solvent, such as DMSO.

In some embodiments, kits may include instructions for employing kit components as well the use of any other reagent not included in the kit. Instructions may include variations that may be implemented.

### Devices

In some embodiments, the AAV particles may delivered to a subject using a device to deliver the AAV particles and a head fixation assembly. The head fixation assembly may be, but is not limited to, any of the head fixation assemblies sold by MRI interventions. As a non-limiting example, the head fixation assembly may be any of the assemblies described in U.S. Pat. Nos. 8,099,150, 8,548,569 and 9,031,636 and International Patent Publication Nos. WO201108495 and WO2014014585, the contents of each of which are incorporated by reference in their entireties. A head fixation assembly may be used in combination with an MRI compatible drill such as, but not limited to, the MRI compatible drills described in International Patent Publication No. WO2013181008 and US Patent Publication No. US20130325012, the contents of which are herein incorporated by reference in its entirety.

In some embodiments, the AAV particles may be delivered using a method, system and/or computer program for positioning apparatus to a target point on a subject to deliver the AAV particles. As a non-limiting example, the method, system and/or computer program may be the methods, systems and/or computer programs described in U.S. Pat. No. 8, 340,743, the contents of which are herein incorporated by reference in its entirety. The method may include: determining a target point in the body and a reference point, wherein the target point and the reference point define a planned trajectory line (PTL) extending through each; determining a visualization plane, wherein the PTL intersects the visualization plane at a sighting point; mounting the guide device relative to the body to move with respect to the PTL, wherein the guide device does not intersect the visualization plane; determining a point of intersection (GPP) between the guide axis and the visualization plane; and aligning the GPP with the sighting point in the visualization plane.

In some embodiments, the AAV particles may be delivered to a subject using a convention-enhanced delivery device. Non-limiting examples of targeted delivery of drugs using convection are described in US Patent Publication Nos, US20100217228, US20130035574 and US20130035660 and International Patent Publication No. WO2013019830 and WO2008144585, the contents of each of which are herein incorporated by reference in their entireties.

In some embodiments, a subject may be imaged prior to, during and/or after delivery of the AAV particles. The imaging method may be a method known in the art and/or described herein, such as but not limited to, magnetic resonance imaging (MRI)). As a non-limiting example, imaging may be used to assess therapeutic effect. As another non-limiting example, imaging may be used for assisted delivery of AAV particles.

In some embodiments, the AAV particles may be delivered using an MRI-guided device. Non-limiting examples of MRI-guided devices are described in U.S. Pat. Nos. 9,055,884, 9,042,958, 8,886,288, 8,768,433, 8,396,532, 8,369,930, 8,374,677 and 8,175,677 and US Patent Application No. US20140024927 the contents of each of which are herein incorporated by reference in their entireties. As a non-limiting example, the MRI-guided device may be able to provide data in real time such as those described in U.S. Pat. Nos. 8,886,288 and 8,768,433, the contents of each of which is herein incorporated by reference in its entirety. As another non-limiting example, the MRI-guided device or system may be used with a targeting cannula such as the systems described in U.S. Pat. Nos. 8,175,677 and 8,374,677, the contents of each of which are herein incorporated by reference in their entireties. As yet another non-limiting example, the MRI-guided device includes a trajectory guide frame for guiding an interventional device as described, for example, in U.S. Pat. No. 9,055,884 and US Patent Application No. US20140024927, the contents of each of which are herein incorporated by reference in their entireties.

In some embodiments, the AAV particles may be delivered using an MRI-compatible tip assembly. Non-limiting examples of MRI-compatible tip assemblies are described in US Patent Publication No. US20140275980, the contents of which is herein incorporated by reference in its entirety.

In some embodiments, the AAV particles may be delivered using a cannula which is MRI-compatible. Non-limiting examples of MRI-compatible cannulas include those taught in International Patent Publication No. WO2011130107, the contents of which are herein incorporated by reference in its entirety. In some embodiments, the cannula or a portion thereof or the tubing associated with the cannula is attached, mounted, glued, affixed or otherwise makes reversible contact with the tissue surrounding the surgical site/field. Such contact may be localized and/or stabilized in one position during all or a portion of the procedure.

In some embodiments, the AAV particles may be delivered using a catheter which is MRI-compatible. Non-limiting examples of MRI-compatible catheters include those taught in International Patent Publication No. WO2012116265, US Patent Publication No. 8,825,133 and US Patent Publication No. US20140024909, the contents of each of which are herein incorporated by reference in their entireties.

In some embodiments, the AAV particles may be delivered using a device with an elongated tubular body and a diaphragm as described in US Patent Publication Nos. US20140276582 and US20140276614, the contents of each of which are herein incorporated by reference in their entireties.

In some embodiments, the AAV particles may be delivered using an MRI compatible localization and/or guidance system such as, but not limited to, those described in US Patent Publication Nos, US20150223905 and US20150230871, the contents of each of which are herein incorporated by reference in their entireties. As a non-limiting example, the MRI compatible localization and/or guidance systems may comprise a mount adapted for fixation to a patient, a targeting cannula with a lumen configured to attach to the mount so as to be able to controllably translate in at least three dimensions, and an elongate probe configured to snugly advance via slide and retract in the targeting cannula lumen, the elongate probe comprising at least one of a stimulation or recording electrode.

in some embodiments, the AAV particles may be delivered to a subject using a trajectory frame as described in US Patent Publication Nos, US20150031982 and US20140066750 and International Patent Publication Nos. WO2015057807 and WO2014039481, the contents of each of which are herein incorporated by reference in their entireties.

In some embodiments, the AAV particles may be delivered to a subject using a gene gun.

### V. Methods and Uses of the Compositions of the Disclosure

### Huntington's Disease (HD)

Huntington's Disease (HD) is a monogenic fatal neurodegenerative disease characterized by progressive chorea, neuropsychiatric and cognitive dysfunction. Huntington's disease is known to be caused by an autosomal dominant triplet (CAG) repeat expansion in the huntingtin (RH) gene, which encodes poly-glutamine at the N-terminus of the HTT protein. This repeat expansion results in a toxic gain of function of HTT and ultimately leads to striatal neurodegeneration which progresses to widespread brain atrophy. Medium spiny neurons of the striatum appear to be especially vulnerable in HD with up to 95% loss, whereas interneurons are largely spared,

Huntington's Disease has a profound impact on quality of life. Symptoms typically appear between the ages of 35-44 and life expectancy subsequent to onset is 10-25 years. In a small percentage of the HD population (˜6%), disease onset occurs prior to the age of 21 with appearance of an akinetic-rigid syndrome. These cases tend to progress faster than those of the later onset variety and have been classified as juvenile or Westphal variant HD. It is estimated that approximately 35,000-70,000 patients are currently suffering from HD in the US and Europe. Currently, only symptomatic relief and supportive therapies are available for treatment of HD, with a cure yet to be identified. Ultimately, individuals with HD succumb to pneumonia, heart failure or other complications such as physical injury from falls.

While not wishing to be bound by theory, the function of the wild type HTT protein may serve as a scaffold to coordinate complexes of other proteins. HTT is a very large protein (67 exons, 3144 amino acids, ˜350kDa) that undergoes extensive post-translational modification and has numerous sites for interaction with other proteins, particularly at its N-terminus (coincidently the region that carries the repeats in HD). HTT localizes primarily to the cytoplasm but has been shown to shuttle into the nucleus where it may regulate gene transcription. It has also been suggested that HTT has a role in vesicular transport and regulating RNA trafficking.

As a non-limiting example, the HT1 protein sequence is SEQ ID NO: 1424 (NCBI NP_002102.4) and the HTT nucleic acid sequence is SEQ ID NO: 1425 (NCBI NM_002111.7).

The mechanisms by which CAG-expanded HTT disrupts normal HTT function and results in neurotoxicity were initially thought to be a disease of haploinsufficiency, this theory was disproven when terminal deletion of the HTT gene in human did not lead to development of HD, suggesting that fully expressed HTT protein is not critical to survival. However, conditional knockout of HTT in mouse led to neurodegeneration, indicating that some amount of HTT is necessary for cell survival. Huntingtin protein is expressed in all cells, though its concentration is highest in the brain where large aggregates of abnormal HTT are fbund in neuronal nuclei. In the brains of HD patients, HTT aggregates into abnormal nuclear inclusions. It is now believed that it is this process of misfolding and aggregating along with the associated protein intermediates (i.e. the soluble species and toxic N-terminal fragments) that result in neurotoxicity. In fact. HD belongs to a family of nine additional human genetic disorders all of which are characterized by CAG-expanded genes and resultant polyglutatnine (poly-Q) protein products with subsequent formation of intraneuronal aggregates. Interestingly, in all of these diseases the length of the expansion correlates with both age of onset and rate of disease progression, with longer expansions linked to greater severity of disease.

Hypotheses on the molecular mechanisms underlying the neurotoxicity of CAG-expanded HTT and its resultant aggregates have been wide ranging, but include, caspase activation, dysregulation of transcriptional pathways, increased production of reactive oxygen species, mitochondrial dysfunction, disrupted axonal transport and/or inhibition of protein degradation systems within the cell. CAG-expanded HTT may not only have a toxic gain of function, but also exert a dominant negative effect by interfering with the normal function of other cellular proteins and processes. HTT has also been implicated in non-cell autonomous neurotoxicity, whereby a cell hosting HTT spreads the HTT to other neurons nearby.

In some embodiments, a subject has fully penetrant HD where the HTT gene has 41 or more CAG repeats (e.g., 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90 or more than 90 CAG repeats)

In some embodiments, a subject has incomplete penetrance where the HTT gene has between 36 and 40 CAG repeats (e.g., 36, 37, 38. 39 and 40 CAG repeats).

Symptoms of HD may include features attributed to CNS degeneration such as, but are not limited to, chorea, dystonia, bradykinesia, incoordination, irritability and depression, problem solving difficulties, reduction in the ability of a person to function in their normal day to day life, diminished speech, and difficulty swallowing, as well as features not attributed to CNS degeneration such as, but not limited to, weight loss, muscle wasting, metabolic dysfunction and endocrine disturbances.

Model systems for studying Huntington's Disease which may be used with the modulatory polynucleotides and AAV particles described herein include, but are not limited to, cell models (e.g., primary neurons and induced pluripotent stem cells), invertebrate models (e.g., drosophila or caenorhabditis elegans), mouse models (e.g., YAC128 mouse model; R6/2 mouse model; BAC and knock-in mouse model), rat models (e.g., BAC) and large mammal models (e.g., mini-pigs, pigs, sheep or monkeys).

Studies in animal models of HD have suggested that phenotypic reversal is feasible, for example, subsequent to gene shut off in regulated-expression models. In a mouse model allowing shut off of expression of a 94-polyglutamine repeat HTT protein, not only was the clinical syndrome reversed but also the intracellular aggregates were resolved. Further, animal models in which silencing of HTT was tested, demonstrated promising results with the therapy being both well tolerated and showing potential therapeutic benefit.

Such siRNA mediated HTT expression inhibition may be used for treating HD. According to the present disclosure, methods for treating and/or ameliorating HD in a patient comprises administering to the patient an effective amount of AAV particles comprising a nucleic acid sequence encoding the siRNA molecules of the present disclosure into cells. The administration of the AAV particles comprising such a nucleic acid sequence will encode the siRNA molecules which cause the inhibition/silence of HTT gene expression.

In some embodiments, the AAV particles described herein may be used to reduce the amount of HTT in a subject or patient in need thereof and thus provides a therapeutic benefit as described herein.

In certain aspects, the symptoms of HD include behavioral difficulties and symptoms such as, but not limited to, apathy or lack of initiative, dysphoria, irritability, agitation or anxiety, poor self-care, poor judgment, inflexibility, disinhibition, depression, suicidal ideation euphoria, aggression, delusions, compulsions, hypersexuality, hallucinations, speech deterioration, slurred speech, difficulty swallowing, weight loss, cognitive dysfunction which impairs executive functions (e.g., organizing, planning, checking or adapting alternatives, and delays in the acquisition of new motor skills), unsteady gait and involuntary movements (chorea). In other aspects, the composition of the present disclosure is applied to one or both of the brain and the spinal cord. In some embodiments, the survival of the subject is prolonged by treating any of the symptoms of HD described herein.

Disclosed in the present disclosure are methods for treating Huntington's Disease (HD) associated with HTT protein in a subject or patient in need of treatment. The method optionally comprises administering to the subject a therapeutically effective amount of a composition comprising at least AAV particles comprising a nucleic acid sequence encoding the siRNA molecules of the present disclosure. As a non-limiting example, the siRNA molecules can silence HTT gene expression, inhibit HTT protein production, and reduce one or more symptoms of HD in the subject such that HD is therapeutically treated.

Methods of treatment of Huntington's Disease

The present disclosure provides AAV particles comprising modulatory polynucleotides encoding siRNA molecules targeting the HTT gene, and methods for their design and manufacture. While not wishing to be bound by a single theory of operability, the AAV particles described herein provide modulatory polynucleotides, including siRNAs, that interfere with HTT expression, including HTT mutant and/or wild-type HTT gene expression. Particularly, the present disclosure employs viral genomes such as adeno-associated viral (AAV) viral genomes comprising modulatory polynucleotide sequences encoding the siRNA molecules of the present disclosure. The AAV vectors comprising the modulatory polynucleotides encoding the siRNA molecules of the present disclosure may increase the delivery of active agents into neurons of interest such as medium spiny neurons of the striatum and cortical neurons. The siRNA duplexes or encoded dsRNA targeting the HTT gene may be able to inhibit HTT gene expression (e.g., mRNA level) significantly inside cells; therefore, reducing HTT expression-induced stress inside the cells such as aggregation of protein and formation of inclusions, increased free radicals, mitochondrial dysfunction and RNA metabolism.

Provided in the present disclosure are methods for introducing the AAV particles comprising a modulatory polynucleotide sequence encoding the siRNA molecules of the present disclosure into cells, the method comprising introducing into said cells any of the AAV particles in an amount sufficient for degradation of target HTT mRNA to occur, thereby activating target-specific RNAi. in the cells. In some aspects, the cells may be stem cells, neurons such as medium spiny or cortical neurons, muscle cells and glial cells such as astrocytes. In another aspect, the cells may be FRhK-4 rhesus macaque (Macaw mulatta) kidney cells, and fibroblasts from HD patients.

In some embodiments, the present disclosure provides methods for treating or ameliorating Huntington's Disease (HD) by administering to a subject or patient in need thereof a therapeutically effective amount of a plasmid or AAV vector described herein.

In some embodiments, the AAV particles comprising modulatory polynucleotides encoding the siRNA molecules of the present disclosure may be used to treat and/or ameliorate for HD.

In some embodiments, the AAV particles comprising modulatory polynucleotides encoding the siRNA molecules of the present disclosure may be used to reduce the cognitive and/or motor decline of a subject with HD, where the amount of decline is determined by a standard evaluation system such as, but not limited to, Unified Huntington's Disease Ratings Scale (UHDRS) and subscores, and cognitive testing.

In some embodiments, the AAV particles comprising modulatory polynucleotides encoding the siRNA molecules of the present disclosure may be used to reduce the motor decline of a subject with HD, where the amount of decline is determined by a standard behavioral test such as, but not limited to, a rotarod test, a balance beam test, and/or an open field test.

In some embodiments, the AAV particles comprising modulatory polynucleotides encoding the siRNA molecules of the present disclosure may be used to reduce the anxiety of a subject with HD, where the amount of anxiety is determined by a standard behavioral test such as, but not limited to an open field test and/or a light/dark box test.

In some embodiments, the AAV particles comprising modulatory polynucleotides encoding the siRNA molecules of the present disclosure may be used to reduce the decline of functional capacity and activities of daily living as measured by a standard evaluation system such as, but not limited to, the total functional capacity (TFC) scale.

In some embodiments, the present disclosure provides methods for treating, or ameliorating Huntington's Disease associated with HTT gene and/or HTT protein in a subject or patient in need of treatment, the method comprising administering to the subject a pharmaceutically effective amount of AAV particles comprising modulatory polynucleotides encoding at least one siRNA duplex targeting the HTT gene, inhibiting HTT gene expression and protein production, and ameliorating symptoms of HD in the subject.

In some embodiments, the AAV vectors of the present disclosure may be used as a method of treating Huntington's disease in a subject or patient in need of treatment, Any method known in the art for defining a subject or patient in need of treatment may be used to identify said subject(s). A subject may have a clinical diagnosis of Huntington's disease, or may be pre-symptomatic. Any known method for diagnosing HD may be utilized, including, but not limited to, cognitive assessments and/or neurological or neuropsychiatric examinations, motor tests, sensory tests, psychiatric evaluations, brain imaging, family history and/or genetic testing.

In some embodiments, HD subject selection is determined with the use of the Prognostic Index for Huntington's Disease, or a derivative thereof (Long ID et al., Movement Disorders, 2017, 32(2), 256-263, the contents of which are herein incorporated by reference in their entirety). This prognostic index uses four components to predict probability of motor diagnosis, (1) total motor score (TMS) from the Unified Huntington's Disease Rating Scale (UHDRS), (2) Symbol Digit Modality Test (SDMT), (3) base-line age, and (4) cytosine-adenine-guanine (CAG) expansion.

In some embodiments, the prognostic index for Huntington's Disease is calculated with the following formula: PI_{HD}=51×TMS+(−34)×SDMT+7×Age×(CAG-34), wherein larger values for PI_{HD} indicate greater risk of diagnosis or onset of symptoms.

In another embodiment, the prognostic index for Huntington's Disease is calculated with the following normalized formula that gives standard deviation (stdev or SD) units to be interpreted in the context of 50% 10-year survival: PIN_{HD}=(PI_{HD}−883)/1044, wherein PIN_{HD}<0 indicates greater than 50% 10-year survival, and PIN_{HD}>0 suggests less than 50% 10-year survival.

In some embodiments, the prognostic index may be used to identify subjects whom will develop symptoms of HD within several years, but that do not yet have clinically diagnosable symptoms. Further, these asymptomatic patients may be selected for and receive treatment using the AAV vectors and compositions of the present disclosure during the asymptomatic period.

In some embodiments, the AAV particles may be administered to a subject who has undergone biomarker assessment. Potential biomarkers in blood for premanifest and early progression of HD include, but are not limited to, 8-OhdG oxidative stress marker, metabolic markers (e.g., creatine kinase, branched-chain amino acids), cholesterol metabolites (e.g., 24-OH cholesterol), immune and inflammatory proteins clusterin, complement components, interleukins 6 and 8), gene expression changes (e.g., transcriptomic markers), endocrine markers (e.g., cortisol, ghrelin and leptin), BDNF, adenosine 2A receptors. Potential biomarkers for brain imaging for premanifest and early progression of HD include, but are not limited to, striatal volume, subcortical white-matter volume, cortical thickness, whole brain and ventricular volumes, functional imaging (e.g., functional MRI), PET (e.g, with fluorodeoxyglucose), and magnetic resonance spectroscopy (e.g., lactate). Apart from measurement of huntingtin, among other potential biomarkers is neurofilament light chain, which is a potential marker of neurodegeneration and may be assessed in biofluids such as cerebrospinal fluid or using neuroimaging approaches. Potential biomarkers for quantitative clinical tools for premanifest and early progression of HD include, but are not limited to, quantitative motor assessments, motor physiological assessments (e.g., transcranial magnetic stimulation), and quantitative eye movement measurements. Non-limiting examples of quantitative clinical biomarker assessments include tongue force variability, metronome-guided tapping, grip force, oculomotor assessments and cognitive tests. Non-limiting examples of multicenter observational studies include PREDICT-HD and TRACK-HD. A subject may have symptoms of HD, diagnosed with HD or may be asymptomatic for HD.

In some embodiments, the AAV particles may be administered to a subject who has undergone biomarker assessment using neuroimaging. A subject may have symptoms of HD, diagnosed with HD or may be asymptomatic for HD.

In some embodiments, the AAV particles may be administered to a subject who is asymptomatic for HD. A subject may be asymptomatic but may have undergone predictive genetic testing or biomarker assessment to determine if they are at risk for HD and/or a subject may have a family member (e.g., mother, father, brother, sister, aunt, uncle, grandparent) who has been diagnosed with HD.

In some embodiments, the AAV particles may be administered to a subject who is in the early stages of HD. In the early stage a subject has subtle changes in coordination, some involuntary movements (chorea), changes in mood such as irritability and depression, problem solving difficulties, reduction in the ability of a person to function in their normal day to day life.

In some embodiments, the AAV particles may be administered to a subject who is in the middle stages of HD. In the middle stage a subject has an increase in the movement disorder, diminished speech, difficulty swallowing, and ordinary activities will become harder to do. At this stage a subject may have occupational and physical therapists to help maintain control of voluntary movements and a subject may have a speech language pathologist.

In some embodiments, the AAV particles may be administered to a subject who is in the late stages of HD. In the late stage, a subject with HD is almost completely or completely dependent on others for care as the subject can no longer walk and is unable to speak. A subject can generally still comprehend language and is aware of family and friends, but choking is a major concern.

In some embodiments, the AAV particles may be used to treat a subject who has the juvenile form of HD which is the onset of HD before the age of 20 years and as early as 2 years.

In some embodiments, the AAV particles may be used to treat a subject with HD who has fully penetrant HD where the HTT gene has 41 or more GAG repeats (e.g., 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90 or more than 90 CAG repeats).

In some embodiments, the AAV particles may be used to treat a subject with HD who has incomplete penetrance where the HTT gene has between 36 and 40 CAG repeats (e.g., 36, 37, 38, 39 and 40 CAG repeats).

In some embodiments, the composition comprising the AAV particles comprising modulatory polynucleotides encoding the siRNA molecules of the present disclosure is administered to the central nervous system of the subject. In other embodiments, the composition comprising the AAV particles comprising modulatory polynucleotides encoding the siRNA molecules of the present disclosure is administered to a tissue of a subject (e.g., brain of the subject).

In some embodiments, the AAV particles comprising modulatory polynucleotides encoding the siRNA molecules of the present disclosure may be delivered into specific types of targeted cells, including, but not limited to, neurons including medium spiny or cortical neurons; glial cells including oligodendrocytes, astrocytes and microglia; and/or other cells surrounding neurons such as T cells.

In some embodiments, the AAV particles comprising modulatory polynucleotides encoding the siRNA molecules of the present disclosure may be delivered to neurons in the striatum and/or neurons of the cortex.

In some embodiments, the composition of the present disclosure for treating HD is administered to the subject or patient in need intravenously, intramuscularly, subcutaneously, intraperitoneally, intraparenchymally, subpially, intrathecally and/or intraventricularly, allowing the siRNA molecules or vectors comprising the siRNA molecules to pass through one or both the blood-brain barrier and the blood spinal cord barrier, or directly access the brain and/or spinal cord. In some aspects, the method includes administering (e.g., intraparenchymal administration, subpial administration, intraventricular administration and/or intrathecal administration) directly to the central nervous system (CNS) of a subject (using, e.g., an infusion pump and/or a delivery scaffold) a therapeutically effective amount of a composition comprising AAV particles encoding the nucleic acid sequence for the siRNA molecules of the present disclosure. The vectors may be used to silence or suppress HTT gene expression, and/or reducing one or more symptoms of HD in the subject such that HD is therapeutically treated.

In some embodiments, the siRNA molecules or the AAV vectors comprising such siRNA molecules may be introduced directly into the central nervous system of the subject, for example, by infusion into the white matter of a subject. While not wishing to be bound by theory, distribution via direct white matter infusion may be independent of axonal transport mechanisms which may be impaired in subjects with Huntington's Disease which means white matter infusion may allow for more transport of the AAV vectors.

In some embodiments, the composition comprising the AAV particles comprising modulatory polynucleotides encoding the siRNA molecules of the present disclosure is administered to the central nervous system of the subject via intraparenchymal injection,

In some embodiments, the AAV particle composition comprising modulatory polynucleotides encoding the siRNA molecules of the present disclosure is administered to the central nervous system of the subject via intraparenchymal injection and intrathecal injection,

In some embodiments, the AAV particle composition comprising modulatory polynucleotides encoding the siRNA molecules of the present disclosure is administered to the central nervous system. of the subject via intraparenchymal injection and intracerebroventricular injection.

In some embodiments, the composition of the present disclosure for treating HD is administered to the subject or patient in need by intraparenchymal administration.

In some embodiments, the AAV particle composition comprising modulatory polynucleotides encoding the siRNA molecules of the present disclosure may be introduced directly into the central nervous system of the subject, for example, by infusion into the putamen.

In some embodiments, the AAV particle composition comprising modulatory polynucleotides encoding the siRNA molecules of the present disclosure may be introduced directly into the central nervous system of the subject, for example, by infusion into the thalamus of a subject. While not wishing to be bound by theory, the thalamus is an area of the brain which is relatively spared in subjects with Huntington's Disease which means it may allow for more widespread cortical transduction via axonal transport of the AAV vectors.

In some embodiments, the AAV particle composition comprising modulatory polynucleotides encoding the siRNA molecules of the present disclosure may be introduced indirectly into the central nervous system of the subject, for example, by intravenous administration.

In some embodiments, AAV particles described herein are administered via putamen and thalamus infusion. Dual infusion into the putamen and thalamus may be independently bilateral or unilateral. As a non-limiting example, AAV particles may be infused into the putamen and thalamus from bath sides of the brain. As another non-limiting example, AAV particles may be infused into the left putamen and left thalamus, or right putamen and right thalamus, As yet another non-limiting example, AAV particles may be infused into the left putamen and right thalamus, or right putamen and left thalamus. Dual infusion may occur consecutively or simultaneously.

### Modulation of HTT Expression

In some embodiments, administration of the AAV particles to a subject will reduce the expression of HTT in a subject and the reduction of expression of the HTT will reduce the effects of HD in a subject.

In some embodiments, the encoded dsRNA once expressed and contacts a cell expressing HTT protein, inhibits the expression of HTT protein by at least 10%, at least 20%, at least 25%, at least 30%, at least 35% or at least 40% or more, such as when assayed by a method as described herein.

In some embodiments, administration of the AAV particles comprising a modulatory polynucleotide sequence encoding a siRNA of the disclosure, to a subject may lower HTT (e.g., mutant HTT, wild-type HTT and/or mutant and wild-type HTT) in a subject. In some embodiments, administration of the AAV particles to a subject may lower wild-type HTT in a subject. In yet another embodiment, administration of the AAV particles to a subject may lower both mutant HTT and wild-type HTT in a subject e mutant and/or wild-type HTT may he lowered by about 10%, 20%, 30%, 40%, 50%, 60%, 70%, 80%, 85%, 90%, 95% and 100%, or at least 10-20%, 10-30%, 10-40%, 10-50%, 10-60%, 10-70%, 10-80%, 10-90%, 10-95%, 10-100%, 20-30%, 20-40%, 20-50%, 20-60%, 20-70%, 20-80%, 20-90%, 20-95%, 20-100%, 40%, 30-50%, 30-60%, 30-70%, 30-80%, 30-90%, 30-95%, 30-100%, 40-504;, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100% in a subject such as, but not limited to, the CNS, a region of the CNS, or a specific cell of the CNS of a subject. The mutant HTT may be lowered by about 10%, 0.20%, 30%, 40%, 50%, 60%, 70%, 80%, 85%, 90%, 95% and 100%, or at least 10-20%, 10-30%, 10-40%, 10-50%, 10-60%, 10-70%, 10-80%, 10-90%, 10-95%, 10-100%, 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%), 60-100%, 70-80%, 70-90%, 70-95%, 70-100%, 80-90%, 80-95%, 80-100%, 90-95%, 90-100% or 95-100% in a subject such as, but not limited to, the CNS, a region of the CNS, or a specific cell of the CNS of a subject. The wild-type HTT may be lowered by about 10%, 20%, 30%, 40%, 50%, 60%, 70%, 80%, 85%, 90%, 95% and 100%, or at least 10-20%, 10-30%, 10-40%, 10-50%, 10-60%, 10-70%, 10-80%, 10-90%, 10-95%, 10-100%,20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%), 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100% in a subject such as, but not limited to, the CNS, a region of the CNS, or a specific cell of the CNS of a subject. The mutant and wild-type HTT may be lowered by about 10%, 20%, 30%, 40%, 50%, 60%, 70%, 80%, 85%, 90%, 95% and 100%, or at least 10-20%, 10-30%, 10-40%, 10-50%, 10-60%, 10-70%, 10-80%, 10-90%, 10-95%, 10-100%, 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100% in a subject such as, but not limited to, the CNS, a region of the CNS, or a specific cell of the CNS of a subject. As a non-limiting example, the AAV particles may lower the expression of HTT by at least 50% in the medium spiny neurons. As a non-limiting example, the vectors, AAV vectors may lower the expression of HTT by at least 40% in the medium spiny neurons. As a non-limiting example, the AAV particles may lower the expression of HTT by at least 40% in the medium spiny neurons of the putamen. As a non-limiting example, AAV particles may lower the expression of HTT by at least 30% in the medium spiny neurons of the putamen. As yet another non-limiting example, the AAV particles may lower the expression of HTT in the putamen and cortex by at least 40%. As yet another non-limiting example, the AAV particles may lower the expression of HTT in the putamen and cortex by at least 30%. As yet another non-limiting example, the AAV particles may lower the expression of HTT in the putamen by at least 30%. As yet another non-limiting example, the AAV particles may lower the expression of HTT in the putamen by at least 30% and cortex by at least 15%.

In some embodiments, the AAV particles may be used to reduce the expression of HTT protein by at least about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 85%, 90%, 95% and 100%, or at least 10-20%, 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 55-60%, 55-70%, 55-80%, 55-90%, 55-95%, 55-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%. As a non-limiting example, the expression of HTT protein expression may be reduced by 50-90%. As a non-limiting example, the expression of HTT protein expression may be reduced by 30-70%.

In some embodiments, the siRNA duplexes or encoded dsRNA may be used to reduce the expression of HTT mRNA by at least about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 85%, 90%, 95% and 100%, or at least 10-30%, 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-85%, 50-90%, 50-95%, 50-100%, 55-60%, 55-70%, 55-80%, 55-90%, 55-95° c, 55-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%. As a non-limiting example, the expression of HTT mRNA may be reduced by 50-90%. As a non-limiting example, the expression of HTT mRNA expression may be reduced by 30-70%. As a non-limiting example, the expression of HTT mRNA expression may be reduced by 40-70%. As a non-limiting example, the expression of HTT mRNA expression may be reduced by 50-80%. As a non-limiting example, the expression of HTT mRNA expression may be reduced by 50-85%. As a non-limiting example, the expression of HTT mRNA expression may be reduced by 60-90%.

In some embodiments, the AAV particles may be used to decrease HTT protein in a subject. The decrease may independently be 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%. 25-85%. 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50- 75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%), 60-95%), 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%. As a non-limiting example, a subject may have a 50% decrease of HTT protein. As a non-limiting example, a subject may have a decrease of 70% of HTT protein and a decrease of 10% of wild type HTT protein. As a non-limiting example, the decrease of HTT in the medium spiny neurons of the putamen may be about 40%. As a non-limiting example, the decrease of HTT in neurons of the caudate may be about 30%. As a non-limiting example, the decrease of HTT in neurons of the thalamus may be about 40%. As a non-limiting example, the decrease of HTT in the cortex may be about 20%. As a non-limiting example, the decrease of HTT in pyramidal neurons of the primary motor and somatosensory cortices may be about 30%. As a non-limiting example, the decrease of HTT in the putamen and cortex may be about 40%. As a non-limiting example, the decrease of HTT in the putamen, caudate and cortex may be about 40%. As a non-limiting example, the decrease of HTT in the putamen, caudate, cortex and thalamus may be about 40%. As a non-limiting example, the decrease of HTT in the medium spiny neurons of the putamen may be between 40%-70%. As a non-limiting example, the decrease of HTT in neurons of the caudate may be between 30%-70%. As a non-limiting example, the decrease of HTT in the putamen and cortex may be between 40%-70%. As a non-limiting example, the decrease of HTT in the putamen, caudate and cortex may be between 40%-70%. As a non-limiting example, the decrease of HTT in the putamen, caudate, cortex and thalamus may be between 40%-80%.

In some embodiments. the AAV particles may be used to decrease wild type HTT protein in a subject. The decrease may independently be 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%. As a non-limiting example, a subject may have a 50% decrease of wild type HTT protein. As a non-limiting example, the decrease of wild type HTT in the medium spiny neurons of the putamen may be about 40%. As a non-limiting example, the decrease of wild type HTT in neurons of the caudate may be about 30%. As a non-limiting example, the decrease of wild type HTT in neurons of the thalamus may be about 40%. As a non-limiting example, the decrease of wild type HTT in the cortex may be about 20%. As a non-limiting example, the decrease of wild type HTT in pyramidal neurons of the primary motor and somatosensory cortices may be about 30%. As a non-limiting example, the decrease of wild type HTT in the putamen and cortex may be about 40%. As a non-limiting example, the decrease of wild type HTT in the putamen, caudate and cortex may be about 40%. As a non-limiting example, the decrease of wild type HYT in the putamen, caudate, cortex and thalamus may be about 40%, As a non-limiting example, the decrease of wild type HTT in the medium spiny neurons of the putamen may be between 40%-70%. As a non-limiting example, the decrease of wild type HTT in neurons of the caudate may be between 30%-70%. As a non-limiting example, the decrease of wild type HTT in the putamen and cortex may be between 40%-70%. As a non-limiting example, the decrease of wild type HTT in the putamen, caudate and cortex may be between 40%-70%. As a non-limiting example, the decrease of wild type HTT in the putamen, caudate, cortex and thalamus may be between 40%-80%.

In some embodiments, the AAV particles may be used to decrease mutant HTT protein in a subject. The decrease may independently be 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 70-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35- 50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%. As a non-limiting example, a subject may have a 50% decrease of mutant HTT protein. As a non-limiting example, the decrease of mutant HTT in the medium spiny neurons of the putamen may be about 40%. As a non-limiting example, the decrease of mutant HTT in neurons of the caudate may be about 30%. As a non-limiting example, the decrease of mutant HTT in neurons of the thalamus may be about 40%. As a non-limiting example, the decrease of mutant HTT in the cortex may be about 20%. As a non-limiting example, the decrease of mutant HTT in pyramidal neurons of the primary motor and somatosensory cortices may be about 30%. As a non-limiting example, the decrease of mutant HTT in the putamen and cortex may be about 40%. As a non-limiting example, the decrease of mutant HTT in the putamen, caudate and cortex may be about 40%. As a non-limiting example, the decrease of mutant HTT in the putamen, caudate, cortex and thalamus may be about 40%. As a non-limiting example, the decrease of mutant HTT in the medium spiny neurons of the putamen may be between 40%-70%. As a non-limiting example, the decrease of mutant HTT in neurons of the caudate may be between 30%-70%. As a non-limiting example, the decrease of mutant HTT in the putamen and cortex may be between 40%-70%. As a non-limiting example, the decrease of mutant HTT in the putamen, caudate and cortex may be between 40%-70%. As a non-limiting example, the decrease of mutant HTT in the putamen, caudate, cortex and thalamus may be between 40%-80%.

In some embodiments, the present disclosure provides methods for inhibitinglsilencing HTT gene expression in a cell. Accordingly, the siRNA duplexes or encoded dsRNA can be used to substantially inhibit HTT gene expression in a cell, in particular in a neuron. In some aspects, the inhibition of HTT gene expression refers to an inhibition by at least about 20%, such as by at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75% 76%, 77%, 78%, 79%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 55-60%, 55-70%, 55-80%, 55-90%, 55-95%, 55-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%. Accordingly, the protein product of the targeted gene may be inhibited by at least about 20%, preferably by at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 85%, 90%. 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%.

In some embodiments, the present disclosure provides methods for inhibiting/silencing HTT gene expression in a cell, in particular in a medium spiny neuron. In some aspects, the inhibition of HTT gene expression refers to an inhibition by at least about 20%, such as by at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 55-60%, 55-70%, 55-80%, 55-90%, 55-95%, 55-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%. Accordingly, the protein product of the targeted gene may be inhibited by at least about 20%, preferably by at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%. 76%, 77%, 78%, 79%, 80%, 85%, 90%, 95% and 100, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 55-60%, 55-70%, 55-80%, 55-90%, 55-95%, 55-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%.

In some embodiments, the present disclosure provides methods for inhibiting/silencing HTT gene expression in a cell, in particular in a glial cell such as, but not limited to, an astrocyte, an oligodendrocyte, or a microglial cell. In some aspects, the inhibition of HTT gene expression refers to an inhibition by at least about 20%, such as by at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 55-60%, 55-70%, 55-80%, 55-90%, 55-95%, 55-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-10000. Accordingly, the protein product of the targeted gene may be inhibited by at least about 20% preferably by at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 55-60%, 55-70%, 55-80%, 55-90%, 55-95%, 55-100%, 60-70%, 60-80%, 60-90%, 60-95%, 60-100%, 70-80%, 70-90%, 70-95%, 70-100%. 80-90%, 80-95%, 80-100%, 90-95%, 90-100% or 95-100%,

In some embodiments, the present disclosure provides methods for inhibiting/silent HTT gene expression in a cell, in particular in an astrocyte. In some aspects, the inhibition of HTT gene expression refers to an inhibition by at least about 20%, such as by at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 55-60%, 55-70%, 55-80%, 55-90%, 55-95%, 55-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%. Accordingly, the protein product of the targeted gene may be inhibited by at least about 20%, preferably by at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 55-60%, 55-70%, 55-80%, 55-90%, 55-95%, 55-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%.

In some embodiments, the present disclosure provides methods for inhibiting/silencing HTT gene expression in a cell, in particular in an oligodendrocyte. In some aspects, the inhibition of HTT gene expression refers to an inhibition by at least about 20%, such as by at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 55-60%, 55-70%, 55-80%, 55-90%, 55-95%, 55-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%. Accordingly, the protein product of the targeted aerie may be inhibited by at least about 20%, preferably by at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 55-60%, 55-70%, 55-80%, 55-90%, 55-95%, 55%-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%.

In some embodiments, the present disclosure provides methods for inhibiting/silencing HTT gene expression in a cell, in particular in a microglial cell. In some aspects, the inhibition of HTT gene expression refers to an inhibition by at least about 20%, such as by at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 55-60%, 55-70%, 55-80%, 55-90%, 55-95%. 55-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%. Accordingly, the protein product of the targeted gene may be inhibited by at least about 20%, preferably by at least about 30%, 31%, 32%, 33%. 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 55-60%, 55-70%, 55-80%, 55-90%, 55-95%, 55-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%.

In some embodiments, the siRNA duplexes or encoded dsRNA may be used to reduce the expression of HTT protein and/or mRNA in at least one region of the CNS such as, but not limited to the midbrain. The expression of HTT protein and/or mRNA is reduced by at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 85%, 90%, 95% and 100%, or at least 20-30%, 20-40%, 20-50%, 20-60%, 20-70%, 20-80%, 20-90%, 20-95%, ?0-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 55-60%, 55-70%, 55-80%, 55-90%, 55-95%, 55-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100% in at least one region of the CNS. As a non-limiting example, the expression of HTT protein and mRNA in the striatum and/or cortex is reduced by 50-90%. As a non-limiting example, the expression of HTT protein and mRNA in the striatum is reduced by 40-50%. As a non-limiting example, the expression of HTT protein and mRNA in the cortex is reduced by 40-50%. As a non-limiting example, the expression of HTT protein and mRNA in the cortex is reduced by 30-70%. As a non-limiting example, the expression of HTT protein and mRNA in the cortex is reduced by at least 30%. As a non-limiting example, the expression of HTT protein and mRNA in the striatum and/or cortex is reduced by 40-70%. As a non-limiting example, the expression of HTT protein and mRNA in the striatum and/or cortex is reduced by 40-50%. As a non-limiting example, the expression of HTT protein and mRNA in the striatum and/or cortex is reduced by 50-70%. As a non-limiting example, the expression of HTT protein and mRNA in the striatum and/or cortex is reduced by 50-60%. As a non-limiting example, the expression of HTT protein and mRNA in the striatum and/or cortex is reduced by 50%. As a non-limiting example, the expression of HTT protein and mRNA in the striatum and/or cortex is reduced by 51%. As a non-limiting example, the expression of HTT protein and mRNA in the striatum and/or cortex is reduced by 52%. As a non-limiting example, the expression of HTT protein and mRNA in the striatum and/or cortex is reduced by 53%. As a non-limiting example, the expression of HTT protein and mRNA in the striatum and/or cortex is reduced by 54%, As a non-limiting example, the expression of HTT protein and mRNA in the striatum and/or cortex is reduced by 55%. As a non-limiting example, the expression of HTT protein and mRNA in the striatum and/or cortex is reduced by 56%. As a non-limiting example, the expression of HTT protein and mRNA in the striatum and/or cortex is reduced by 57%. As a non-limiting example, the expression of HTT protein and mRNA in the striatum and/or cortex is reduced by 58%. As a non-limiting example, the expression of HTT protein and mRNA in the striatum and/or cortex is reduced by 59%. As a non-limiting example, the expression of HTT protein and mRNA in the striatum and/or cortex is reduced by 60%. As a non-limiting example, the expression of HTT protein and mRNA in the striatum, thalamus, and/or cortex is reduced by at least 20%. As a non-limiting example, the expression of HTT protein and mRNA in the striatum, thalamus, and/or cortex is reduced by 30%. As a non-limiting example, the expression of HTT protein and mRNA in the striatum, thalamus, and/or cortex is reduced by 30-70%. As a non-limiting example, the expression of HTT protein and mRNA in the striatum, thalamus, and/or cortex is reduced by 40-80%. As a non-limiting example, the expression of HTT protein and mRNA in the striatum, thalamus, and/or cortex is reduced by 40-70%. As a non-limiting example, the expression of HTT protein and mRNA in the striatum, thalamus, and/or cortex is reduced by 40-60%. As a non-limiting example, the expression of HTT protein and mRNA in the striatum, thalamus, and/or cortex is reduced by 50-80%. As a non-limiting example, the expression of HTT protein and mRNA in the striatum, thalamus, and/or cortex is reduced by 50-70%.

In some embodiments, the present disclosure provides methods for inhibiting/silencing HTT gene expression in a cell, in particular in a pyramidal neuron of the primary motor cortex or primary somatosensory cortex or temporal cortex. in some aspects, the inhibition of HTT gene expression refers to an inhibition by at least about 20%, such as by at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 55-60%, 55-70%, 55-80%, 55-90%, 55-95%, 55-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%, Accordingly, the protein product of the targeted gene may be inhibited by at least about 20%, preferably by at least about 30%, 31%. 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30.-95%, 30-100%, 40-50%, 40-60%, 40-70%, 40-80%, 40-90%, 40-95%, 40-100%, 50-60%, 50-70%, 50-80%, 50-90%, 50-95%, 50-100%, 55-60%, 55-70%, 55-80%, 55-90%, 55-95%, 55-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%.

In some embodiments, the siRNA duplexes or encoded dsRNA may be used to reduce the expression of HTT protein and/or mRNA in at least one region of the CNS such as, but not limited to the forebrain. The expression of HTT protein and/or mRNA is reduced by at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 55-60%, 55-70%, 55-80%, 55-90%, 55-95%, 55-100%, 60-70%, 60-80%, 60-90%, 60-95%, 60-100%, 70-80%, 70-90%, 70-95%, 70-100%, 80-90%, 80-95%, 80-100%, 90-95%, 90-100%, or 95-100% in at least one region of the CNS. As a non-limiting example, the expression of HTT protein and/or mRNA in the putamen is reduced by 50-90%. As anon-limiting example, the expression of HTT protein and/or mRNA in the striatum is reduced by 40-50%. As a non-limiting example, the expression of HTT protein and/or mRNA in the cot/ex is reduced by 40-50%. As a non-limiting example, the expression of HTT protein and/or mRNA in the cortex is reduced by 30-70%.

As a non-limiting example, the expression of HTT protein and/or mRNA in the striatum and/or cortex is reduced by 40-70%. As a non-limiting example, the expression of HTT protein and/or mRNA in the striatum and/or cortex is reduced by 40-50%. As a non-limiting example, the expression of HTT protein and/or mRNA in the striatum and/or cortex is reduced by 50-70%. As a non-limiting example, the expression of HTT protein and/or mRNA in the striatum and/or cortex is reduced by 50-60%. As a non-limiting example, the expression of HTT protein and/or mRNA in the striatum and/or cortex is reduced by 50%. As a non-limiting example, the expression of HTT protein and/or mRNA in the striatum and/or cortex is reduced by 51%. As a non-limiting example, the expression of HTT protein and/or mRNA in the striatum and/or cortex is reduced by 52%. As a non-limiting example, the expression of HTT protein and/or mRNA in the striatum and/or cortex is reduced by 53%. As a non-limiting example, the expression of HTT protein and/or mRNA in the striatum and/or cortex is reduced by 54%. As a non-limiting example, the expression of HTT protein and/or mRNA in the striatum and/or cortex is reduced by 55%. As a non-limiting example, the expression of HTT protein and/or mRNA in the striatum and/or cortex is reduced by 56%. As a non-limiting example, the expression of HTT protein and/or mRNA in the striatum and/or cortex is reduced by 57%. As a non-limiting example, the expression of HTT protein and/or mRNA in the striatum and/or cortex is reduced by 58%. As a non-limiting example, the expression of HTT protein and/or mRNA in the striatum and/or cortex is reduced by 59%. As a non-limiting example, the expression of HTT protein and/or mRNA in the striatum and/or cortex is reduced by 60%. As a non-limiting example, the expression of HTT protein and/or mRNA in the striatum and/or cortex is reduced by 61%. As a non-limiting example, the expression of HTT protein and/or mRNA in the striatum and/or cortex is reduced by 62%. As a non-limiting example, the expression of HTT protein and/or mRNA in the striatum and/or cortex is reduced by 63%. As a non-limiting example, the expression of HTT protein and/or mRNA in the striatum and/or cortex is reduced by 64%. As a non-limiting example, the expression of HTT protein and/or mRNA in the striatum and/or cortex is reduced by 65%, As a non-limiting example, the expression of HTT protein and/or mRNA in the striatum and/or cortex is reduced by 66%. As a non-limiting example, the expression of HTT protein and/or mRNA in the striatum and/or cortex is reduced by 67%. As a non-limiting example, the expression of HTT protein and/or mRNA in the striatum and/or cortex is reduced by 68%. As a non-limiting example, the expression of HTT protein and/or mRNA in the striatum and/or cortex is reduced by 69%. As a non-limiting example, the expression of HTT protein and/or mRNA in the striatum and/or cortex is reduced by 70%.

In some embodiments, the siRNA duplexes or encoded dsRNA may be used to reduce the expression of HTT protein and/or mRNA in the striatum. The expression of HTT protein and/or mRNA is reduced by at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%. 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%. As a non-limiting example, the expression of HTT protein and/or mRNA in the striatum is reduced by 40-50%. As a non-limiting example, the expression of HTT protein and/or mRNA in the striatum is reduced by 30-70%. As a non-limiting example, the expression of HTT protein and/or mRNA in the striatum is reduced by at least 30%. As a non-limiting example, the expression of HTT protein and/or mRNA in the striatum is reduced by 40-70%, As a non-limiting example, the expression of HTT protein and/or mRNA in the striatum is reduced by 40-50%. As a non-limiting example, the expression of HTT protein and/or mRNA in the striatum is reduced by 50-70%. As a non-limiting example, the expression of HTT protein and/or mRNA in the striatum is reduced by 50-60%, As a non-limiting example, the expression of HTT protein and/or mRNA in the striatum is reduced by 50%. As a non-limiting example, the expression of HTT protein and/or mRNA in the striatum is reduced by 51%. As a non-limiting example, the expression of HTT protein and/or mRNA in the striatum is reduced by 52%. As a non-limiting example, the expression of HTT protein and/or mRNA in the striatum is reduced by 53%. As a non-limiting example, the expression of HTT protein and/or mRNA in the striatum is reduced by 54%. As a non-limiting example, the expression of HTT protein and/or mRNA in the striatum is reduced by 55%. As a non-limiting example, the expression of HTT protein and/or mRNA in the striatum is reduced by 56%. As a non-limiting example, the expression of HTT protein and/or mRNA in the striatum is reduced by 57%. As a non-limiting example, the expression of HTT protein and/or mRNA in the striatum is reduced by 58%. As a non-limiting example, the expression of HTT protein and/or mRNA in the striatum is reduced by 59%. As a non-limiting example, the expression of HTT protein and/or mRNA in the striatum is reduced by 60%. As a non-limiting example, the expression of HTT protein and/or mRNA in the striatum is reduced by 61%. As a non-limiting example, the expression of HTT protein and/or mRNA in the striatum is reduced by 62%. As a non-limiting example, the expression of HTT protein and/or mRNA in the striatum is reduced by 63%. As a non-limiting example, the expression of HTT protein and/or mRNA in the striatum is reduced by 64%. As a non-limiting example, the expression of HTT protein and/or mRNA in the striatum is reduced by 65%. As a non-limiting example, the expression of HTT protein and/or mRNA in the striatum is reduced by 66%. As a non-limiting example, the expression of HTT protein and/or mRNA in the striatum is reduced by 67%. As a non-limiting example, the expression of HTT protein and/or mRNA in the striatum is reduced by 68%. As a non-limiting example, the expression of HTT protein and/or mRNA in the striatum is reduced by 69%. As a non-limiting example, the expression of HTT protein and/or mRNA in the striatum is reduced by 70%.

In some embodiments, the AAV particles comprising modulatory polynucleotides encoding the siRNA molecules of the present disclosure may be used to suppress HTT protein in neurons and/or astrocytes of the striatum and/or the cortex. As a non-limiting example, the suppression of HTT protein is in medium spiny neurons of the striatum and/or neurons of the cortex. As a non-limiting example, the suppression of HTT protein is in medium spiny neurons of the striatum and/or pyramidal neurons of the primary motor cortex and primary somatosensory cortex.

In some embodiments, the AAV particles comprising modulatory polynucleotides encoding the siRNA molecules of the present disclosure may be used to suppress HTT protein in neurons and/or astrocytes of the striatum and/or the cortex and reduce associated neuronal toxicity. The suppression of HTT protein in the neurons and/or astrocytes of the striatum and/or the cortex may be, independently, suppressed by 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 70-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%. The reduction of associated neuronal toxicity may be 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80% 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%.

In some embodiments, the siRNA duplexes or encoded dsRNA may be used to reduce the expression of HTT protein and/or snRNA in the cortex. The expression of HTT protein and/or mRNA is reduced by at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%. As a non-limiting; example, the expression of HTT protein and/or mRNA in the cortex is reduced by 40-50%, As a non-limiting example, the expression of HTT protein and/or mRNA in the cortex is reduced by 30-70%. As a non-limiting example, the expression of HTT protein and/or mRNA in the cortex is reduced by at least 30%.

As a non-limiting example, the expression of HTT protein and/or mRNA in the cortex is reduced by 40-70%. As a non-limiting example, the expression of HTT protein and/or mRNA in the cortex is reduced by 40-50%. As a non-limiting example, the expression of HTT protein and/or mRNA in the cortex is reduced by 50-70%. As a non-limiting example, the expression of HTT protein and/or mRNA in the cortex is reduced by 50-60%. As a non-limiting example, the expression of HTT protein and/or mRNA in the cortex is reduced by 50%, As a non-limiting example, the expression of HTT protein and/or mRNA in the cortex is reduced by 51%. As a non-limiting example, the expression of HTT protein and/or mRNA in the cortex is reduced by 52%. As a non-limiting example, the expression of HTT protein and/or mRNA in the cortex is reduced by 53%. As a non-limiting example, the expression of HTT protein and/or mRNA in the cortex is reduced by 54%. As a non-limiting example, the expression of HTT protein and/or mRNA in the cortex is reduced by 55%. As a non-limiting example, the expression of HTT protein and/or mRNA in the cortex is reduced by 56%. As a non-limiting; example, the expression of HTT protein and/or mRNA in the cortex is reduced by 57%. As a non-limiting example, the expression of HTT protein and/or mRNA in the cortex is reduced by 58%. As a non-limiting example, the expression of HTT protein and/or mRNA in the cortex is reduced by 59%. As a non-limiting example, the expression of HTT protein and/or mRNA in the cortex is reduced by 60%.

In some embodiments, the siRNA duplexes or encoded dsRNA may be used to reduce the expression of HTT protein and/or mRNA in the motor cortex. The expression of HTT protein and/or mRNA is reduced by at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%. 71%, 72%, 73%, 74%, 75%, 76%, 77%, 78%, 79%, 80%, 85%, 90%, 95% and 100%, or at least 20-30%, 20-40%, 20-50%, 20-60%, 20-70%, 20-80%, 20-90%, 70-95%, 70-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%. As a non-limiting example, the expression of HTT protein and/or mRNA in the motor cortex is reduced by 20-30%. As a non-limiting example, the expression of HTT protein and/or mRNA in the motor cortex is reduced by 40-50%. As a non-limiting example, the expression of HTT protein and/or mRNA in the motor cortex is reduced by 30-70%. As a non-limiting example, the expression of HTT protein and/or mRNA in the motor cortex is reduced by at least 30%. As a non-limiting example, the expression of HTT protein and/or mRNA in the motor cortex is reduced by 40-70%. As a non-limiting example, the expression of HTT protein and/or mRNA in the motor cortex is reduced by 40-50%. As a non-limiting example, the expression of HTT protein and/or mRNA in the motor cortex is reduced by 50-70%. As a non-limiting example, the expression of HTT protein and/or mRNA in the motor cortex is reduced by 50-60%. As a non-limiting example, the expression of HTT protein and/or mRNA in the motor cortex is reduced by 50%, As a non-limiting example, the expression of HTT protein and/or mRNA in the motor cortex is reduced by 51%. As a non-limiting example, the expression of HTT protein and/or mRNA in the motor cortex is reduced by 52%. As a non-limiting example, the expression of HTT protein and/or mRNA in the motor cortex is reduced by 53%. As a non-limiting example, the expression of HTT protein and/or mRNA in the motor cortex is reduced by 54%. As a non-limiting example, the expression of HTT protein and/or mRNA in the motor cortex is reduced by 55%. As a non-limiting example, the expression of KU protein and/or mRNA in the motor cortex is reduced by 56%. As a non-limiting example, the expression of HTT protein and/or mRNA in the motor cortex is reduced by 57%. As a non-limiting example, the expression of HTT protein and/or mRNA in the motor cortex is reduced by 58%. As a non-limiting example, the expression of HTT protein and/or mRNA in the motor cortex is reduced by 59%. As a non-limiting example, the expression of HTT protein and/or mRNA in the motor cortex is reduced by 60%.

In some embodiments, the siRNA duplexes or encoded dsRNA may be used to reduce the expression of HTT protein and/or mRNA in the somatosensory cortex, The expression of HTT protein and/or mRNA is reduced by at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%. As a non-limiting example, the expression of HTT protein and/or mRNA in the somatosensory cortex is reduced by 20-30%.

As a non-limiting example, the expression of HTT protein and/or mRNA in the somatosensory cortex is reduced by 40-50%. As a non-limiting example, the expression of HTT protein and/or mRNA in the somatosensory cortex is reduced by 30-70%. As a non-limiting example, the expression of HTT protein and/or mRNA in the somatosensory cortex is reduced by at least 30%. As a non-limiting example, the expression of HTT protein and/or mRNA in the somatosensory cortex is reduced by 40-70%. As a non-limiting example, the expression of HTT protein and/or mRNA in the somatosensory cortex is reduced by 40-50%. As a non-limiting example, the expression of HTT protein and/or mRNA in the somatosensory cortex is reduced by 50-70%. As a non-limiting example, the expression of HTT protein and/or mRNA in the somatosensory cortex is reduced by 50-60%. As a non-limiting; example, the expression of HUI: protein and/or mRNA in the somatosensory cortex is reduced by 50%. As a non-limiting example, the expression of HTT protein and/or mRNA in the somatosensory cortex is reduced by 51%. As a non-limiting example, the expression of HTT protein and/or mRNA in the somatosensory cortex is reduced by 52%. As a non-limiting example, the expression of HTT protein and/or mRNA in the somatosensory cortex is reduced by 53%. As a non-limiting example, the expression of HTT protein and/or mRNA in the somatosensory cortex is reduced by 54%. As a non-limiting example, the expression of HTT protein and/or mRNA in the somatosensory cortex is reduced by 55%. As a non-limiting example, the expression of HTT protein and/or mRNA in the somatosensory cortex is reduced by 56%. As a non-limiting example, the expression of HTT protein and/or mRNA in the somatosensory cortex is reduced by 57%. As a non-limiting example, the expression of HTT protein and/or mRNA in the somatosensory cortex is reduced by 58%. As a non-limiting example, the expression of HTT protein and/or mRNA in the somatosensory cortex is reduced by 59%. As a non-limiting example, the expression of HTT protein and/or mRNA in the somatosensory cortex is reduced by 60%.

In some embodiments, the siRNA duplexes or encoded dsRNA may be used to reduce the expression of HTT protein and/or snRNA in the temporal cortex. The expression of HTT protein and/or mRNA is reduced by at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%. As a non-limiting example, the expression of HTT protein and/or mRNA in the temporal cortex is reduced by 40-50%. As a non-limiting example, the expression of HTT protein and/or mRNA in the temporal cortex is reduced by 30-70%. As a non-limiting example, the expression of HTT protein and/or mRNA in the temporal cortex is reduced by at least 30%. As a non-limiting example, the expression of HTT protein and/or mRNA in the temporal cortex is reduced by 40-70%. As a non-limiting example, the expression of HTT protein and/or mRNA in the temporal cortex is reduced by 40-50%. As a non-limiting example, the expression of HTT protein and/or mRNA in the temporal cortex is reduced by 50-70%. As a non-limiting example, the expression of HTT protein and/or mRNA in the temporal cortex is reduced by 50-60%. As a non-limiting example, the expression of HTT protein and/or mRNA in the temporal cortex is reduced by 50%. As a non-limiting example, the expression of HTT protein and/or mRNA in the temporal cortex is reduced by 51%. As a non-limiting example, the expression of HTT protein and/or mRNA in the temporal cortex is reduced by 52%. As a non-limiting example, the expression of HTT protein and/or mRNA in the temporal cortex is reduced by 53%. As a non-limiting example, the expression of HTT protein and/or mRNA in the temporal cortex is reduced by 54%. As a non-limiting example, the expression of HTT protein and/or mRNA in the temporal cortex is reduced by 55%. As a non-limiting example, the expression of HTT protein and/or mRNA in the temporal cortex is reduced by 56%. As a non-limiting example, the expression of HTT protein and/or mRNA in the temporal cortex is reduced by 57%. As a non-limiting example, the expression of HTT protein and/or mRNA in the temporal cortex is reduced by 58%. As a non-limiting example, the expression of HTT protein and/or mRNA in the temporal cortex is reduced by 59%. As a non-limiting example, the expression of HTT protein and/or mRNA in the temporal cortex is reduced by 60%,

In some embodiments, the siRNA duplexes or encoded dsRNA may be used to reduce the expression of HTT protein and/or mRNA in the motor cortex, the somatosensory cortex, and the temporal cortex when combined together. The expression of HTT protein and/or mRNA is reduced by at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%. As a non-limiting example, the expression of HTT protein and/or mRNA in the motor cortex, the somatosensory cortex, and the temporal cortex when combined together is reduced by 40-50%. As a non-limiting example, the expression of HTT protein and/or mRNA in the motor cortex, the somatosensory cortex, and the temporal cortex when combined together is reduced by 30-70%. As a non-limiting example, the expression of HTT protein and/or mRNA in the motor cortex, the somatosensory cortex, and the temporal cortex when combined together is reduced by at least 30%. As a non-limiting example, the expression of HTT protein and/or mRNA in the motor cortex, the somatosensory cortex, and the temporal cortex when combined together is reduced by 40-70%. As a non-limiting example, the expression of HTT protein and/or mRNA in the motor cortex, the somatosensory cortex, and the temporal cortex when combined together is reduced by 40-50%. As a non-limiting example, the expression of HTT protein and/or mRNA in the motor cortex, the somatosensory cortex, and the temporal cortex when combined together is reduced by 50-70%. As a non-limiting example, the expression of HTT protein and/or mRNA in the motor cortex, the somatosensory cortex, and the temporal cortex when combined together is reduced by 50-60%. As a non-limiting example, the expression of HTT protein and/or mRNA in the motor cortex, the somatosensory cortex, and the temporal cortex when combined together is reduced by 50%. As a non-limiting example, the expression of HTT protein and/or mRNA in the motor cortex, the somatosensory cortex, and the temporal cortex when combined together is reduced by 51%. As a non-limiting example, the expression of HTT protein and/or mRNA in the motor cortex, the somatosensory cortex, and the temporal cortex when combined together is reduced by 52%. As a non-limiting example, the expression of HTT protein and/or mRNA in the motor cortex, the somatosensory cortex, and the temporal cortex when combined together is reduced by 53%. As a non-limiting example, the expression of HTT protein and/or snRNA in the motor cortex, the somatosensory cortex, and the temporal cortex when combined together is reduced by 54%. As a non-limiting example, the expression of HTT protein and/or mRNA in the motor cortex, the somatosensory cortex, and the temporal cortex when combined together is reduced by 55%. As a non-limiting example, the expression of HTT protein and/or mRNA in the motor cortex, the somatosensory cortex, and the temporal cortex when combined together is reduced by 56%. As a non-limiting example, the expression of HTT protein and/or mRNA in the motor cortex, the somatosensory cortex, and the temporal cortex when combined together is reduced by 57%. As a non-limiting example, the expression of HTT protein and/or mRNA in the motor cortex, the somatosensory cortex, and the temporal cortex when combined together is reduced by 58%. As a non-limiting example, the expression of HTT protein and/or mRNA in the motor cortex, the somatosensory cortex, and the temporal cortex when combined together is reduced by 59%. As a non-limiting example, the expression of HTT protein and/or mRNA in the temporal cortex is reduced by 60%.

In some embodiments, the siRNA duplexes or encoded dsRNA may be used to reduce the expression of HTT protein and/or mRNA in the putamen. The expression of HTT protein and/or mRNA is reduced by at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%. 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 55-60%, 55-70%, 55-80%, 55-90%, 55-95%, 55-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100% in the putamen. As a non-limiting example, the expression of HTT protein and/or mRNA in the putamen is reduced by 40-70%. As a non-limiting example, the expression of HTT protein and/or mRNA in the putamen is reduced by 30-40%. As a non-limiting example, the expression of HTT protein and/or mRNA in the putamen is reduced by 40-50%. As a non-limiting example, the expression of HTT protein and/or mRNA in the putamen is reduced by 50-80%. As a non-limiting example, the expression of HTT protein and/or mRNA in the putamen is reduced by 50-70%. As a non-limiting example, the expression of HTT protein and/or mRNA in the putamen is reduced by 50-60%. As a non-limiting example, the expression of HTT protein and/or mRNA in the putamen is reduced by 60-70%. As a non-limiting example, the expression of HTT protein and/or mRNA in the putamen is reduced by 50%. As a non-limiting example, the expression of HTT protein and/or mRNA in the putamen is reduced by 51%. As a non-limiting example, the expression of HTT protein and/or mRNA in the putamen is reduced by 52%. As a non-limiting example, the expression of HTT protein and/or mRNA in the putamen is reduced by 53%. As a non-limiting example, the expression of HTT protein and/or mRNA in the putamen is reduced by 54%. As a non-limiting example, the expression of HTT protein and/or mRNA in the putamen is reduced by 55%. As a non-limiting example, the expression of HTT protein and/or mRNA in the putamen is reduced by 56%. As a non-limiting example, the expression of HTT protein and/or mRNA in the putamen is reduced by 57%. As a non-limiting example, the expression of HTT protein and/or mRNA in the putamen is reduced by 58%. As a non-limiting example, the expression of HTT protein and/or mRNA in the putamen is reduced by 59%. As a non-limiting example, the expression of HTT protein and/or mRNA in the putamen is reduced by 60%. As a non-limiting example, the expression of HTT protein and/or mRNA in the putamen is reduced by 61%. As a non-limiting example, the expression of HTT protein and/or mRNA in the putamen is reduced by 62%. As a non-limiting example, the expression of HTT protein and/or mRNA in the putamen is reduced by 63%. As a non-limiting example, the expression of HTT protein and/or mRNA in the putamen is reduced by 64%. As a non-limiting example, the expression of HTT protein and/or mRNA in the putamen is reduced by 65%. As a non-limiting example, the expression of HTT protein and/or mRNA in the putamen is reduced by 66%, As a non-limiting example, the expression of HTT protein and/or mRNA in the putamen is reduced by 67%. As a non-limiting example, the expression of HTT protein and/or mRNA in the putamen is reduced by 68%. As a non-limiting example, the expression of HTT protein and/or mRNA in the putamen is reduced by 69%. As a non-limiting example, the expression of HTT protein and/or mRNA in the putamen is reduced by 70%.

In some embodiments, the siRNA duplexes or encoded dsRNA may be used to reduce the expression of HTT protein and/or mRNA in the caudate. The expression of HTT protein and/or mRNA is reduced by at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-85%, 50-90%, 50-95%, 50-100%, 55-60%, 55-70%, 55-80%, 55-90%, 55-95%, 55-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100% in the caudate. As a non-limiting example, the expression of HTT protein and/or mRNA in the caudate is reduced by 40-70%. As a non-limiting example, the expression of HTT protein and/or mRNA in the caudate is reduced by 40-50%. As a non-limiting example, the expression of HTT protein and/or mRNA in the caudate is reduced by 50-85%. As a non-limiting example, the expression of HTT protein and/or mRNA in the caudate is reduced by 50-80%. As a non-limiting example, the expression of HTT protein and/or mRNA in the caudate is reduced by 50-70%. As a non-limiting example, the expression of HTT protein and/or mRNA in the caudate is reduced by 50-60%. As a non-limiting example, the expression of HTT protein and/or mRNA in the caudate is reduced by 60-70%. As a non-limiting example, the expression of HTT protein and/or mRNA in the caudate is reduced by 50%. As a non-limiting example, the expression of HTT protein and/or mRNA in the caudate is reduced by 51%. As a non-limiting example, the expression of HTT protein and/or mRNA in the caudate is reduced by 52%. As a non-limiting example, the expression of HTT protein and/or mRNA in the caudate is reduced by 53%. As a non-limiting example, the expression of HTT protein and/or mRNA in the caudate is reduced by 54%. As a non-limiting example, the expression of HTT protein and/or mRNA in the caudate is reduced by 55%. As a non-limiting example, the expression of HTT protein and/or mRNA in the caudate is reduced by 56%. As a non-limiting example, the expression of HTT protein and/or mRNA in the caudate is reduced by 57%. As a non-limiting example, the expression of HTT protein and/or mRNA in the caudate is reduced by 58%. As a non-limiting example, the expression of HTT protein and/or mRNA in the caudate is reduced by 59%. As a non-limiting example, the expression of HTT protein and/or mRNA in the caudate is reduced by 60%. As a non-limiting example, the expression of HTT protein and/or mRNA in the caudate is reduced by 61%. As a non-limiting example, the expression of HTT protein and/or mRNA in the caudate is reduced by 62%. As a non-limiting example, the expression of HTT protein and/or mRNA in the caudate is reduced by 63%. As a non-limiting example, the expression of HTT protein and/or mRNA in the caudate is reduced by 64%. As a non-limiting example, the expression of HTT protein and/or mRNA in the caudate is reduced by 65%. As a non-limiting example, the expression of HTT protein and/or mRNA in the caudate is reduced by 66%. As a non-limiting example, the expression of HTT protein and/or mRNA in the caudate is reduced by 67%. As a non-limiting example, the expression of HTT protein and/or mRNA in the caudate is reduced by 68%. As a non-limiting example, the expression of HTT protein and/or mRNA in the caudate is reduced by 69%. As a non-limiting example, the expression of HTT protein and/or mRNA in the caudate is reduced by 70%.

In some embodiments, the siRNA duplexes or encoded dsRNA may be used to reduce the expression of HTT protein and/or mRNA in the thalamus. The expression of HTT protein and/or mRNA is reduced by at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-85%, 50-90%, 50-95%, 50-100%, 55-60%, 55-70%, 55-80%, 55-90%, 55-95%, 55-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100% in the thalamus. As a non-limiting example, the expression oft-ITT protein and/or mRNA in the thalamus is reduced by at least 30%. As a non-limiting example, the expression of HTT protein and/or mRNA in the thalamus is reduced by 40-70%. As a non-limiting limiting example, the expression of HTT protein and/or mRNA in the thalamus is reduced by 40-80%. As a non-limiting example, the expression of HTT protein and/or mRNA in the thalamus is reduced by 60-90%. As a non-limiting example, the expression of HTT protein and/or mRNA in the thalamus is reduced by 60-80%. As a non-limiting example, the expression of HTT protein and/or mRNA in the thalamus is reduced by 60-70%. As a non-limiting example, the expression of HTT protein and/or mRNA in the thalamus is reduced by 60%. As a non-limiting example, the expression of HU protein and/or mRNA in the thalamus is reduced by 61%. As a non-limiting example, the expression of HTT protein and/or mRNA in the thalamus is reduced by 62%. As a non-limiting example, the expression of HTT protein and/or mRNA in the thalamus is reduced by 63%. As a non-limiting example, the expression of HTT protein and/or mRNA in the thalamus is reduced by 64%. As a non-limiting example, the expression of HTT protein and/or mRNA in the thalamus is reduced by 65%, As a non-limiting example, the expression of HTT protein and/or mRNA in the thalamus is reduced by 66%. As a non-limiting example, the expression of HTT protein and/or snRNA in the thalamus is reduced by 67%. As a non-limiting example, the expression of HTT protein and/or mRNA in the thalamus is reduced by 68%. As a non-limiting example, the expression of HTT protein and/or mRNA in the thalamus is reduced by 69%. As a non-limiting example, the expression of HTT protein and/or mRNA in the thalamus is reduced by 70%. As a non-limiting example, the expression of HTT protein and/or mRNA in the thalamus is reduced by 71%, As a non-limiting example, the expression of HTT protein and/or mRNA in the thalamus is reduced by 72%. As a non-limiting example, the expression of HTT protein and/or snRNA in the thalamus is reduced by 73%. As a non-limiting example, the expression of HTT protein and/or mRNA in the thalamus is reduced by 74%. As a non-limiting example, the expression of HTT protein and/or mRNA in the thalamus is reduced by 75%. As a non-limiting example, the expression of HTT protein and/or mRNA in the thalamus is reduced by 76%. As a non-limiting example, the expression of HTT protein and/or mRNA in the thalamus is reduced by 77%, As a non-limiting example, the expression of HTT protein and/or mRNA in the thalamus is reduced by 78%. As a non-limiting example, the expression of HTT protein and/or snRNA in the thalamus is reduced by 79%. As a non-limiting example, the expression of HTT protein and/or mRNA in the thalamus is reduced by 80%.

In some embodiments. AAV particles encoding siRNA duplexes, or pharmaceutical compositions thereof, have a half maximal effective concentration (EC₅₀) of about 1-300 VG/cell. The half maximal effective concentration (EC₅₀), as used herein, refers to the concentration of AAV vectors encoding siRNA duplexes that produces 50% reduction in HTT expression in a cell. HTT expression may be HTT mRNA or protein expression. AAV particles encoding siRNA duplexes, or pharmaceutical compositions thereof, may have an EC₅₀ of 1-10, 1-20, 1-30, 1-40, 1-50, 10-20, 10-30, 10-40, 10-50, 10-60, 15-30, 20-30, 20-40, 20-50, 20-60, 20-70, 30-40, 30-50, 30-60, 30-70, 30-80, 35-50, 40-50, 40-60, 40-70, 40-80, 40-90, 50-60, 50-70, 50-80, 50-90, 50-100, 60-70, 60-80, 60-90, 60-100, 70-90, 70-100, 70-120, 80-100, 80-120, 80-140, 90-120, 90-150, 90-180, 100-120, 100-150, 100-180, 100-200, 120-160, 120-180, 150-200, 200-250, 200-300, or 250-300 VG/cell. For example, the AAV particles encoding siRNA duplexes, or pharmaceutical compositions thereof, may have an EC₅₀ of about 20, 21, 22, 23, 24, 25, 26, 27, 28, 29, 30, 31, 32, 33, 34, 35, 36, 37, 38, 39, 40, 41, 42, 43, 44, 45 46, 47, 48, 49, 50, 60, 70, 80, 90, 100, 110, 120, 130, 140, 150, 160, 170, 180, 190, 200, 250, or 300 VG/cell. As a non-limiting example, AAV particles encoding siRNA duplexes, or pharmaceutical compositions thereof, may have an EC₅₀ of about 35-50 VG/cell in the putamen. As another non-limiting example, AAV particles encoding siRNA duplexes, or pharmaceutical compositions thereof, may have an EC₅₀ of about 15-30 G/cell in the caudate.

### HTT Protein Aggregates and Cells

In some embodiments, administration of the AAV particles to a subject will modulate the quantity, e.g., level and/or number, of HTT protein aggregates and/or cells in a subject. As a non-limiting example, the HTT protein aggregates may be nuclear HTT protein aggregates. The nuclear HTT protein aggregates may be EM48-positive nuclear HTT protein aggregates, which express EM48 mRNA and/or protein. In particular embodiments, the EM48 mRNA and/or protein may be detected and measured as an index of, or proxy for, the quantity of nuclear protein aggregates. As a non-limiting example, the EM48 mRNA and/or protein may be detected and measured using any method known in the art, including quantitative reverse transcription polymerase chain reaction (RT-qPCR) and immunohistochemistry. The cells may be nervous system cells such as, but not limited, to neurons, medium-sized spiny neurons, and glial cells, e.g., astrocytes, oligodendrocytes, and/or microglia.

In some embodiments, administration of the AAV particles to a subject will modulate the quantity of neurons in a subject. As a non-limiting example, the neuron is a neuronal nuclear antigen (NeuN)-positive neuron, which expresses NeuN mRNA and/or protein. In particular embodiments, the NeuN snRNA and/or protein may be detected and measured as an index of, or proxy for, the quantity of neurons. As a non-limiting example, the NeuN mRNA and/or protein may be detected and measured using any method known in the art, including quantitative reverse transcription polymerase chain reaction (RT-qPCR) and immunohistochemistry.

In some embodiments, administration of the AAV particles to a subject will modulate the quantity of medium-size spiny neurons in a subject. As a non-limiting example, the medium-size spiny neuron is a dopamine- and cAMP-regulated phosphoprotein. Mr 32 kDa (DARPP32)-positive medium-size spiny neuron, which expresses DARPP32 mRNA and/or protein. In particular embodiments, the DARPP32 mRNA and/or protein may be detected and measured as an index of or proxy for, the quantity of medium-size spiny neurons. As a non-limiting example, the DARPP32 mRNA and/or protein may be detected and measured using any method known in the art, including quantitative reverse transcription polymerase chain reaction (RT-qPCR) and immunohistochemistry.

In some embodiments, administration of the AAV particles to a subject will modulate the quantity of astrocytes in a subject. As a non-limiting example, the astrocyte is a glial fibrillary acidic protein (GFAP)-positive astrocyte, which expresses GFAP mRNA and/or protein. In particular embodiments, the GFAP mRNA and/or protein may be detected and/or measured as an index of, or proxy for, the quantity of astrocytes. As a non-limiting example, the GFAP mRNA and/or protein may be detected and/or measured using any method known in the art, including quantitative reverse transcription polymerase chain reaction (RT-qPCR) and immunohistochemistry.

In some embodiments, administration of the AAV particles to a subject will modulate the level and/or number of microglia in a subject. As a non-limiting example, the microglia is a ionized calcium binding adaptor molecule 1 (Iba1)-positive microglia, which expresses Iba1 mRNA and/or protein. in particular embodiments, the Iba1 mRNA and/or protein may be detected and/or measured as an index of, or proxy for, the quantity of astrocytes. As a non-limiting example, the GFAP mRNA and/or protein may be detected and/or measured using any method known in the art, including quantitative reverse transcription polymerase chain reaction (RT-qPCR) and immunohistochemistry.

### Solo and Combination Therapy

In some embodiments, the present composition is administered as a solo therapeutic or combination therapeutics for the treatment of HD.

In some embodiments, the pharmaceutical composition of the present disclosure is used as a solo therapy. In other embodiments, the pharmaceutical composition of the present disclosure is used in combination therapy. The combination therapy may be in combination with one or more neuroprotective agents such as small molecule compounds, growth factors and hormones which have been tested for their neuroprotective effect on neuron degeneration.

The AAV particles encoding siRNA duplexes targeting the HTT gene may be used in combination with one or more other therapeutic agents. By “in combination with,” it is not intended to imply that the agents must be administered at the same time and/or formulated for delivery together, although these methods of delivery are within the scope of the present disclosure. Compositions can be administered concurrently with, prior to, or subsequent to, one or more other desired therapeutics or medical procedures. In general, each agent will be administered at a dose and/or on a time schedule determined for that agent.

Therapeutic agents that may be used in combination with the AAV particles encoding the nucleic acid sequence for the siRNA molecules of the present disclosure can be small molecule compounds which are antioxidants, anti-inflammatory agents, anti-apoptosis agents, calcium regulators, anti-glutamatergic agents, structural protein inhibitors, compounds involved. in muscle function, and compounds involved in metal ion regulation.

Compounds tested for treating HD which may be used in combination with the vectors described herein include, but are not limited to, dopamine-depleting agents (e.g., tetrabenazine for chorea), benzodiazepines (e.g., clonazepam for myoclonus, chorea, dystonia, rigidity, and/or spasticity), anticonvulsants (e.g., sodium valproate and levetiracetam for myoclonus), amino acid precursors of dopamine (e.g., levodopa for rigidity which is particularly associate with juvenile HD or young adult-onset parkinsonian phenotype), skeletal muscle relaxants (e.g., baclofen, tizanidine for rigidity and/or spasticity), inhibitors for acetylcholine release at the neuromuscular junction to cause muscle paralysis (e.g., botulinum toxin for bruxism and/or dystonia), atypical neuroleptics (e.g., olanzapine and quetiapine for psychosis and/or irritability, risperidone, sulpiride and haloperidol for psychosis, chorea and/or irritability, clozapine for treatment-resistant psychosis, aripiprazole for psychosis with prominent negative symptoms), agents to increase ATP/cellular energetics (e.g., creatine), selective serotonin reuptake inhibitors (SSRIs) (e.g., citalopram, fluoxetine, paroxetine, sertraline, mirtazapine, venlafaxine for depression, anxiety, obsessive compulsive behavior and/or irritability). hypnotics (e.g., xopiclone and/or zolpidem for altered sleep-wake cycle), anticonvulsants (e.g., sodium valproate and carbamazepine for mania or hypomania) and mood stabilizers (e.g., lithium for mania or hypomania).

Neurotrophic factors may be used in combination therapy with the AAV particles encoding the nucleic acid sequence for the siRNA molecules of the present disclosure for treating HD. Generally, a neurotrophic factor is defined as a substance that promotes survival, growth, differentiation, proliferation and /or maturation of a neuron, or stimulates increased activity of a neuron. In some embodiments, the present methods further comprise delivery of one or more trophic factors into the subject or patient in need of treatment. Trophic factors may include, but are not limited to, IGF-I, GDNF, BDNF, CTNF, VEGF, Colivelin, Xaliproden, Thyrotrophin-releasing hormone and ADNF, and variants thereof.

In some aspects, the AAV particles comprising modulatory polynucleotides encoding the siRNA duplex targeting the fill gene may be co-administered with AAV vectors expressing neurotrophic factors such as AAV-IGF-I (See e.g., Vincent et al., Neuromo/ecular medicine, 2004, 6, 79-85; the content of which is incorporated herein by reference in its entirety) and AAV-GDNF (See e.g., Wang et al., J Neurosci., 2002, 22. 6920-6928; the content of which is incorporated herein by reference in its entirety).

### VI. Definitions

At various places in the present disclosure, substituents or properties of compounds of the present disclosure are disclosed in groups or in ranges. It is specifically intended that the present disclosure include each and every individual or sub combination of the members of such groups and ranges.

Unless stated otherwise, the following terms and phrases have the meanings described below. The definitions are not meant to be limiting in nature and serve to provide a clearer understanding of certain aspects of the present disclosure.

About: As used herein, the term “about” means +/−10% of the recited value.

Adeno-associated virus: The term “adeno-associated virus” or “AAV” as used herein refers to any vector which comprises or derives from components of an adeno-associated vector and is suitable to infect mammalian cells, preferably human cells. The term AAV vector typically designates an AAV type viral particle or virion comprising a payload. The AAV vector may be derived from various serotypes, including combinations of serotypes (i.e., “pseudotyped” AAV) or from various genomes (e.g., single stranded or self-complementary). In addition, the AAV vector may be replication defective and/or targeted.

AAV Particle: As used herein, an “AAV particle” is a virus which includes a capsid and a viral genome with at least one payload region and at least one ITR region. AAV particles of the present disclosure may be produced recombinantly and may be based on adeno-associated virus (AAV) parent or reference sequences. AAV particle may be derived from any serotype, described herein or known in the art, including combinations of serotypes (i.e., “pseudotyped” AAV) or from various genomes (e.g., single stranded or self-complementary). In addition, the AAV particle may be replication defective and/or targeted.

Activity: As used herein, the term “activity” refers to the condition in which things are happening or being done. Compositions of the present disclosure may have activity and this activity may involve one or more biological events.

Administering: As used herein, the term “administering” refers to providing a pharmaceutical agent or composition to a subject.

Administered in combination: As used herein, the term “administered in combination” or “combined administration” means that two or more agents are administered to a subject at the same time or within an interval such that there may be an overlap of an effect of each agent on the patient. In certain embodiments, they are administered within about 60, 30, 15, 10, 5, or 1 minute of one another. In certain embodiments, the administrations of the agents are spaced sufficiently closely together such that a combinatorial (e.g., a synergistic) effect is achieved. Amelioration: As used herein, the tern “amelioration” or “ameliorating” refers to a lessening of severity of at least one indicator of a condition or disease. For example, in the context of neurodegeneration disorder, amelioration includes the reduction of neuron loss. Animal: As used herein, the term “animal” refers to any member of the animal kingdom. In certain embodiments, “animal” refers to humans at any stage of development. In certain embodiments, “animal” refers to non-human animals at any stage of development. in certain embodiments, the non-human animal is a mammal (e.g., a rodent, a mouse, a rat, a rabbit, a monkey, a dog, a cat, a sheep, cattle, a primate, or a pig). In certain embodiments, animals include, but are not limited to, mammals, birds, reptiles, amphibians, fish, and worms. In certain embodiments, the animal is a transgenic animal, genetically-engineered animal, or a clone. Ant/sense strand: As used herein, the term “the antisense strand” or “the first strand” or “the guide strand” of a siRNA molecule refers to a strand that is substantially complementary to a section of about 10-50 nucleotides, about 15-30, 16-25, 18-23 or 19-22 nucleotides of the mRNA of the gene targeted for silencing. The antisense strand or first strand has sequence sufficiently complementary to the desired target mRNA sequence to direct target-specific silencing, e.g., complementarity sufficient to trigger the destruction of the desired target mRNA by the RNAi machinery or process.

Approximately: As used herein, the term “approximately” or “about,” as applied to one or more values of interest, refers to a value that is similar to a stated reference value. In certain embodiments, the term “approximately” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: As used herein, the terms “associated with,” “conjugated,” “linked,” “attached,” and “tethered,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. An “association” need not be strictly through direct covalent chemical bonding. It may also suggest ionic or hydrogen bonding or a hybridization-based connectivity sufficiently stable such that the “associated” entities remain physically associated.

Baculoviral expression vector (BEV): As used herein a BEV is a baculoviral expression vector, i.e., a polynucleotide vector of haculoviral origin. Systems using BEVs are known as haculoviral expression vector systems (BEVSs).

mBEV or modified BEV: As used herein, a modified BEV is an expression vector of baculoviral origin which has been altered from a starting BEV (whether wild type or artificial) by the addition and/or deletion and/or duplication and/or inversion of one or more: genes; gene fragments; cleavage sites; restriction sites; sequence regions; sequence(s) encoding a payload or gene of interest; or combinations of the foregoing.

Bifunctional: As used herein, the term “bifunctional” refers to any substance, molecule or moiety which is capable of or maintains at least two functions. The functions may affect the same outcome or a different outcome. The structure that produces the function may be the same or different.

BIIC: As used herein a BIIC is a baculoviral infected insect cell.

Biocompatible: As used herein, the term “biocompatible” means compatible with living cells, tissues, organs or systems posing little to no risk of injury, toxicity or rejection by the immune system.

Biodegradable: As used herein, the term “biodegradable” means capable of being broken down into innocuous products by the action of living things.

Biologically active: As used herein, the phrase “biologically active” refers to a characteristic of any substance that has activity in a biological system and/or organism. For instance, a substance that, when administered to an organism, has a biological effect on that organism, is considered to be biologically active. In particular embodiments, an AAV particle of the present disclosure may be considered biologically active if even a portion of the encoded payload is biologically active or mimics an activity considered biologically relevant.

Capsid As used herein, the term “capsid” refers to the protein shell of a virus particle.

Codon optimized: As used herein, the terms “codon optimized” or “codon optimization” refers to a modified nucleic acid sequence which encodes the same amino acid sequence as a parent/reference sequence, but which has been altered such that the codons of the modified nucleic acid sequence are optimized or improved for expression in a particular system (such as a particular species or group of species). As a non-limiting example, a nucleic acid sequence which includes an AAV capsid protein can be codon optimized for expression in insect cells or in a particular insect cell such Spodoptera frugiperda cells. Codon optimization can be completed using methods and databases known to those in the art.

Complementary and substantially complementary: As used herein, the term. “complementary” refers to the ability of polynucleotides to form base pairs with one another. Base pairs are typically formed by hydrogen bonds between nucleotide units in antiparallel polynucleotide strands. Complementary polynucleotide strands can form base pair in the Watson-Crick manner (e.g., A to T, A to U, C to G), or in any other manner that allows for the formation of duplexes. As persons skilled in the art are aware, when using RNA as opposed to DNA, uracil rather than thymine is the base that is considered to be complementary to adenosine. However, when a U is denoted in the context of the present disclosure, the ability to substitute a T is implied, unless otherwise stated. Perfect complementarity or 100% complementarity refers to the situation in which each nucleotide unit of one polynucleotide strand can form hydrogen bond with a nucleotide unit of a second polynucleotide strand. Less than perfect complementarity refers to the situation in which some, but not all, nucleotide units of two strands can form hydrogen bond with each other. For example, for two 20-mers, if only two base pairs on each strand can form hydrogen bond with each other, the polynucleotide strands exhibit 10% complementarity. In the same example, if 18 base pairs on each strand can form hydrogen bonds with each other, the polynucleotide strands exhibit 90% complementarity. As used herein, the term “substantially complementary” means that the siRNA has a sequence (e.g., in the antisense strand) which is sufficient to bind the desired target mRNA, and to trigger the RNA silencing of the target mRNA.

Compound: Compounds of the present disclosure include all of the isotopes of the atoms occurring in the intermediate or final compounds. “Isotopes” refers to atoms having the same atomic number but different mass numbers resulting from a different number of neutrons in the nuclei. For example, isotopes of hydrogen include tritium and deuterium.

The compounds and salts of the present disclosure can be prepared in combination with solvent or water molecules to form solvates and hydrates by routine methods.

Conditionally active: As used herein, the term “conditionally active” refers to a mutant or variant of a wild-type polypeptide, wherein the mutant or variant is more or less active at physiological conditions than the parent polypeptide. Further, the conditionally active polypeptide may have increased or decreased activity at aberrant conditions as compared to the parent polypeptide. A conditionally active polypeptide may be reversibly or irreversibly inactivated at normal physiological conditions or aberrant conditions.

Conserved: As used herein, the term “conserved” refers to nucleotides or amino acid residues of a polynucleotide sequence or polypeptide sequence, respectively, that are those that occur unaltered in the same position of two or more sequences being compared. Nucleotides or amino acids that are relatively conserved are those that are conserved amongst more related sequences than nucleotides or amino acids appearing elsewhere in the sequences.

In certain embodiments, two or more sequences are said to be “completely conserved” if they are 100% identical to one another. In certain embodiments, two or more sequences are said to be “highly conserved” if they are at least 70% identical, at least 80% identical, at least 90% identical, or at least 95% identical to one another. In certain embodiments, two or more sequences are said to be “highly conserved” if they are about 70% identical, about 80% identical, about 90% identical, about 95%, about 98%, or about 99% identical to one another. In certain embodiments, two or more sequences are said to be “conserved” if they are at least 30% identical, at least 40% identical, at least 50% identical, at least 60% identical, at least 70% identical, at least 80% identical, at least 90% identical, or at least 95% identical to one another. In certain embodiments, two or more sequences are said to be “conserved” if they are about 30% identical, about 40% identical, about 50% identical, about 60% identical, about 70% identical, about 80% identical, about 90% identical, about 95% identical, about 98% identical, or about 99% identical to one another. Conservation of sequence may apply to the entire length of a polynucleotide or polypeptide or may apply to a portion, region or feature thereof.

Control Elements: As used herein, “control elements”, “regulatory control elements” or “regulatory sequences” refers to promoter regions, polyadenylation signals, transcription termination sequences, upstream regulatory domains, origins of replication, internal ribosome entry sites (“IRES”), enhancers, and the like, which provide for the replication, transcription and translation of a coding sequence in a recipient cell. Not all of these control elements need always he present as long as the selected coding sequence is capable of being replicated, transcribed and/or translated in an appropriate host cell.

Control/ed Release: As used herein, the term “controlled release” refers to a pharmaceutical composition or compound release profile that conforms to a particular pattern of release to affect a therapeutic outcome.

Cytostatic: As used herein, “cytostatic” refers to inhibiting, reducing, suppressing the growth, division, or multiplication of a cell (e.g., a mammalian cell (e.g., a human cell)), bacterium, virus, fungus, protozoan, parasite, prion, or a combination thereof.

Cytotoxic: As used herein. “cytotoxic” refers to killing or causing injurious, toxic, or deadly effect on a cell (e.g., a mammalian cell (e.g., a human cell)), bacterium, virus, fungus, protozoan, parasite, prion, or a combination thereof.

Delivery: As used herein, “delivery” refers to the act or manner of delivering an AAV particle, a compound, substance, entity, moiety, cargo or payload.

Delivery Agent: As used herein, “delivery agent” refers to any substance which facilitates, at least in part, the in vivo delivery of an AAV particle to targeted cells.

Destabilized: As used herein, the term “destable,” “destabilize,” or “destabilizing region” means a region or molecule that is less stable than a starting, wild-type or native form of the same region or molecule.

Detectable label: As used herein, “detectable label” refers to one or more markers, signals, or moieties which are attached, incorporated or associated with another entity that is readily detected by methods known in the art including radiography, fluorescence, chemiluminescence, enzymatic activity, absorbance and the like. Detectable labels include radioisotopes, fluorophores, chromophores, enzymes, dyes, metal ions, ligands such as biotin, avidin, streptavidin and haptens, quantum dots, and the like, Detectable labels may be located at any position in the peptides or proteins disclosed herein. They may be within the amino acids, the peptides, or proteins, or located at the N- or C-termini.

Digest: As used herein, the term “digest” means to break apart into smaller pieces or components. When referring to polypeptides or proteins, digestion results in the production of peptides.

Distal: As used herein, the term “distal” means situated away from the center or away from a point or region of interest.

Dosing regimen: As used herein, a “dosing regimen” is a schedule of administration or physician determined regimen of treatment, prophylaxis, or palliative care.

Encapsulate: As used herein, the term “encapsulate” means to enclose, surround or encase.

Engineered: As used herein, embodiments of the present disclosure are “engineered” when they are designed to have a feature or property, whether structural or chemical, that varies from a starting point, wild type or native molecule,

Effective Amount: As used herein, the term “effective amount” of an agent is that amount sufficient to effect beneficial or desired results, for example, clinical results, and, as such, an “effective amount” depends upon the context in which it is being applied. For example, in the context of administering an agent that treats HD, an effective amount of an agent is, for example, an amount sufficient to achieve treatment, as defined herein, of HD, as compared to the response obtained without administration of the agent.

Expression: As used herein, “expression” of a nucleic acid sequence refers to one or more of the following events: (1) production of an RNA template from a DNA sequence (e.g., by transcription); (2) processing of an RNA. transcript (e.g., by splicing, editing, 5′ cap formation, and/or 3′ end processing); (3) translation of an RNA into a polypeptide or protein; and (4) post-translational modification of a polypeptide or protein.

Feature: As used herein, a “feature” refers to a characteristic, a property, or a distinctive element.

Formulation: As used herein, a “formulation” includes at least one AAV particle and a delivery agent or excipient.

Fragment: A “fragment,” as used herein, refers to a portion. For example, fragments of proteins may include polypeptides obtained by digesting full-length protein isolated from cultured cells.

Functional: As used herein, a “functional” biological molecule is a biological molecule in a form in which it exhibits a property and/or activity by which it is characterized.

Gene expression: The term “gene expression” refers to the process by which a nucleic acid sequence undergoes successful transcription and in most instances translation to produce a protein or peptide. For clarity, when reference is made to measurement of “gene expression”, this should be understood to mean that measurements may be of the nucleic acid product of transcription, e.g., RNA or mRNA or of the amino acid product of translation, e.g., polypeptides or peptides. Methods of measuring the amount or levels of RNA, mRNA, polypeptides and peptides are well known in the art.

Homology: As used herein, the term “homology” refers to the overall relatedness between polymeric molecules, e.g between polynucleotide molecules (e.g. DNA molecules and/or RNA molecules) and/or between polypeptide molecules. In certain embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). In accordance with the present disclosure, two polynucleotide sequences are considered to be homologous if the polypeptides they encode are at least about 50%, 60%, 70%, 80%, 90%, 95%, or even 99% for at least one stretch of at least about 20 amino acids. In certain embodiments, homologous polytrucleotide sequences are characterized by the ability to encode a stretch of at least 4-5 uniquely specified amino acids. For polynucleotide sequences less than 60 nucleotides in length, homology is determined by the ability to encode a stretch of at least 4-5 uniquely specified amino acids. In accordance with the present disclosure, two protein sequences are considered to be homologous if the proteins are at least about 50%, 60%, 70%, 80%, or 90% identical for at least one stretch of at least about 20 amino acids.

Heterologous Region: As used herein the term “heterologous region” refers to a region which would not be considered a homologous region.

Homologous Region: As used herein the term “homologous region” refers to a region which is similar in position, structure, evolution origin, character, form or function.

Identity: As used herein, the term “identity” refers to the overall relatedness between polymeric molecules, e.g., between polynucleotide molecules (e.g. DNA molecules and/or RNA molecules) and/or between polypeptide molecules. Calculation of the percent identity of two polynucleotide sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: lnfhnnatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo. H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP. BLASTN, and FASTA Altschul, S. F. et al.,1 J. Molec. Biol., 215, 403 (1990)).

Inhibit expression of a gene: As used herein, the phrase “inhibit expression of a gene” means to cause a reduction in the amount of an expression product of the gene. The expression product can be an RNA transcribed from the gene e.g., an mRNA) or a polypeptide translated from an mRNA transcribed from the gene. Typically, a reduction in the level of an mRNA results in a reduction in the level of a polypeptide translated therefrom. The level of expression may be determined using standard techniques for measuring mRNA or protein.

In vitro: As used herein, the term “in vitro” refers to events that occur in an artificial environment, e.g., in a test tube or reaction vessel, in cell culture, in a Petri dish, etc., rather than within an organism (e.g., animal, plant, or microbe).

In vivo: As used herein, the term “in vivo” refers to events that occur within an organism (e.g, animal, plant, or microbe or cell or tissue thereof).

isolated: As used herein, the term “isolated” refers to a substance or entity that has been separated from at least some of the components with which it was associated (whether in nature or in an experimental setting). Isolated substances may have varying levels of purity in reference to the substances from which they have been associated. Isolated substances and/or entities may be separated from at least about 10%, about 20%, about 30%, about 40%, about 50%, about 60%, about 70%, about 80%, about 90%, or more of the other components with which they were initially associated. In certain embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

Substantially isolated: By “substantially isolated” is meant that a substance is substantially separated from the environment in which it was formed or detected. Partial separation can include, for example, a composition enriched in the substance or AAV particles of the present disclosure. Substantial separation can include compositions containing at least about 50%, at least about 60%, at least about 70%, at least about 80%, at least about 90%, at least about 95%, at least about 97%, or at least about 99% by weight of the compound of the present disclosure, or salt thereof. Methods for isolating compounds and their salts are routine in the art.

Linker: As used herein “linker” refers to a molecule or group of molecules which connects two molecules. A linker may be a nucleic acid sequence connecting two nucleic acid sequences encoding two different polypeptides. The linker may or may not be translated. The linker may be a cleavable linker.

MicroRNA (miRNA) binding site: As used herein, a microRNA (miRNA) binding site represents a nucleotide location or region of a nucleic acid transcript to which at least the “seed” region of a miRNA hinds.

Modified: As used herein “modified” refers to a changed state or structure of a molecule of the present disclosure. Molecules may be modified in many ways including chemically, structurally, and functionally. As used herein, embodiments of the disclosure are “modified” when they have or possess a feature or proper, whether structural or chemical, that varies from a starting point, wild type or native molecule.

Mutation: As used herein, the term “mutation” refers to any changing of the structure of a gene, resulting in a variant (also called “mutant”) form that may be transmitted to subsequent generations. Mutations in a gene may be caused by the alternation of single base in DNA, or the deletion, insertion, or rearrangement of larger sections of genes or chromosomes.

Naturally Occurring: As used herein, “naturally occurring” or “wild-type” means existing in nature without artificial aid, or involvement of the hand of man.

Neurodegeneration: As used herein, the term “neurodegeneration” refers to a pathologic state which results in neural cell death. A large number of neurological disorders share neurodegeneration as a common pathological state. For example, Alzheimer's disease, Parkinson's disease, Huntington's disease, and amyotrophic lateral sclerosis (ALS) all cause chronic neurodegeneration, which is characterized by a slow, progressive neural cell death over a period of several years, whereas acute neurodegeneration is characterized by a sudden onset of neural cell death as a result of ischemic, such as stroke, or trauma, such as traumatic brain injury, or as a result of axonal transection by demyelination or trauma caused, for example, by spinal cord injury or multiple sclerosis. In some neurological disorders, mainly one type of neuronal cell is degenerative, for example, medium spiny neuron degeneration in early HD.

Non-human vertebrate: As used herein, a “non-human vertebrate” includes all vertebrates except Homo sapiens, including wild and domesticated species. Examples of non-human vertebrates include, but are not limited to, mammals, such as alpaca, banteng, bison, camel, cat, cattle, deer, dog, donkey, gayal, goat, guinea pig, horse, llama, mule, pig, rabbit, reindeer, sheep water buffalo, and yak.

Nucleic Acid: As used herein, the term “nucleic acid”, “polynucleotide” and “oligonucleotide” refer to any nucleic acid polymers composed of either polydeoxyribonucleotides (containing 2-deoxy-D-ribose), or polyribonucleotides (containing D-ribose), or any other type of polynucleotide which is an N glycoside of a purine or pyrimidine base, or modified purine or pyrimidine bases. There is no intended distinction in length between the term “nucleic acid”, “polynucleotide” and “oligonucleotide”, and these terms will be used interchangeably. These terms refer only to the primary structure of the molecule. Thus, these terms include double- and single-stranded DNA, as well as double- and single stranded RNA.

Off-target: As used herein, “off target” refers to any unintended effect on any one or more target, gene, or cellular transcript.

Open reading frame: As used herein, “open reading frame” or “ORE” refers to a sequence which does not contain a stop codon within the given reading frame, other than at the end of the reading frame.

Operably linked: As used herein, the phrase “operably linked” refers to a functional connection between two or more molecules, constructs, transcripts, entities, moieties or the like.

Patient: As used herein, “patient” refers to a subject who may seek or be in need of treatment, requires treatment, is receiving treatment, will receive treatment, or a subject who is under care by a trained professional for a particular disease or condition.

Payload: As used herein, “payload” or “payload region” refers to one or more polynucleotides or polynucleotide regions encoded by or within a viral genome or an expression product of such polynucleotide or polynucleotide region, e.g., a transgene, a polynucleotide encoding a polypeptide or multi-polypeptide, or a modulatory nucleic acid or regulatory nucleic acid.

Payload construct: As used herein, “payload construct” is one or more vector construct which includes a polynucleotide region encoding or comprising a payload that is flanked on one or both sides by an inverted terminal repeat (ITR) sequence. The payload construct presents a template that is replicated in a viral production cell to produce a therapeutic viral genome.

Payload construct vector: As used herein, “payload construct vector” is a vector encoding or comprising a payload construct, and regulatory regions for replication and expression of the payload construct in bacterial cells.

Payload construct expression vector: As used herein, a “payload construct expression vector” is a vector encoding or comprising a payload construct and which further comprises one or more polynucleotide regions encoding or comprising components for viral expression in a viral replication cell.

Peptide: As used herein, “peptide” is less than or equal to 50 amino acids long, e.g., about 5, 10, 15, 20, 25, 30, 35, 40, 45, or 50 amino acids long.

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable excipients: The phrase “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscannellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Pharmaceutically acceptable salts: The present disclosure also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulthnate, benzene sulthnic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulthnate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile can be used. Lists of suitable salts are found in Remington's Pharmaceutical Sciences, 17^{th} ed., Mack Publishing Company, Easton, Pa., 1985, p. 1418, Pharmaceutical Salts: Properties, Selection, and Use, P.H. Stahl and C.G. Weimuth (eds.), Wiley-VCR, 2008, and Berge et al., Journal of Pharmaceutical Science, 66, 1-19 (1977), each of which is incorporated herein by reference in its entirety.

Pharmaceutically acceptable solvate: The term “pharmaceutically acceptable solvate,” as used herein, means a compound of the present disclosure wherein molecules of a suitable solvent are incorporated in the crystal lattice. A suitable solvent is physiologically tolerable at the dosage administered. For example, solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate,”

Pharmacokinetic: As used herein, “pharmacokinetic” refers to any one or more properties of a molecule or compound as it relates to the determination of the fate of substances administered to a living organism. Pharmacokinetics is divided into several areas including the extent and rate of absorption, distribution, metabolism and excretion. This is commonly referred to as ADME where: (A) Absorption is the process of a substance entering the blood circulation; (D) Distribution is the dispersion or dissemination of substances throughout the fluids and tissues of the body; (M) Metabolism (or Biotransformation) is the irreversible transformation of parent compounds into daughter metabolites; and (E) Excretion (or Elimination) refers to the elimination of the substances from the body. In rare cases, some drugs irreversibly accumulate in body tissue.

Physicochemical: As used herein, “physicochemical” means of or relating to a physical and/or chemical property.

Preventing: As used herein, the term “preventing” or “prevention” refers to partially or completely delaying onset of an infection, disease, disorder and/ or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

Proliferate: As used herein, the term “proliferate” means to grow, expand or increase or cause to grow, expand or increase rapidly. “Proliferative” means having the ability to proliferate. “Anti-proliferative” means having properties counter to or inapposite to proliferative properties.

Prophylactic: As used herein, “prophylactic” refers to a therapeutic or course of action used to prevent the spread of disease.

Prophylaxis: As used herein, a “prophylaxis” refers to a measure taken to maintain health and prevent the spread of disease.

Protein of interest: As used herein, the terms “proteins of interest” or “desired proteins” include those provided herein and fragments, mutants, variants, and alterations thereof.

Proximal: As used herein, the term “proximal” means situated nearer to the center or to a point or region of interest.

Purified: As used herein, “purify,” “purified,” “purification” means to make substantially pure or clear from unwanted components, material defilement, admixture or imperfection. “Purified” refers to the state of being pure. “Purification” refers to the process of making pure.

Region: As used herein, the term “region” refers to a zone or general area, In certain embodiments, when referring to a protein or protein module, a region may include a linear sequence of amino acids along the protein or protein module or may include a three-dimensional area, an epitope and/or a cluster of epitopes. In certain embodiments, regions include terminal regions. As used herein, the term “terminal region” refers to regions located at the ends or termini of a given agent. When referring to proteins, terminal regions may include N- and/or C-termini. N-termini refer to the end of a protein comprising an amino acid with a free amino group. C-termini refer to the end of a protein comprising an amino acid with a free carboxyl group. N- and/or C-terminal regions may there for include the N- and/or C-termini as well as surrounding amino acids. In certain embodiments, N- and/or C-terminal regions include from about 3 amino acid to about 30 amino acids, from about 5 amino acids to about 40 amino acids, from about 10 amino acids to about 50 amino acids, from about 20 amino acids to about 100 amino acids and/or at least 100 amino acids. In certain embodiments, N-terminal regions may include any length of amino acids that includes the N-terminus but does not include the C-terminus. In certain embodiments, C-terminal regions may include any length of amino acids, which include the C-terminus, but do not include the N-terminus.

In certain embodiments, when referring to a polynucleotide, a region may include a linear sequence of nucleic acids along the polynucleotide or may include a three-dimensional area, secondary structure, or tertiary structure. In certain embodiments, regions include terminal regions. As used herein, the term “terminal region” refers to regions located at the ends or termini of a given agent. When referring to polynucleotides, terminal regions may include 5′ and 3′ termini. 5′ termini refer to the end of a polynucleotide comprising a nucleic acid with a free phosphate group. 3′ termini refer to the end of a polynucleotide comprising a nucleic acid with a free hydroxyl group. 5′ and 3′ regions may there for include the 5′ and 3′ termini as well as surrounding nucleic acids. In certain embodiments, 5′ and 3′ terminal regions include from about 9 nucleic acids to about 90 nucleic acids, from about 15 nucleic acids to about 120 nucleic acids, from about 30 nucleic acids to about 150 nucleic acids, from about 60 nucleic acids to about 300 nucleic acids and/or at least 300 nucleic acids. In certain embodiments, 5′ regions may include any length of nucleic acids that includes the 5′ terminus but does not include the 3′ terminus. In certain embodiments, 3′ regions may include any length of nucleic acids, which include the 3′ terminus, but does not include the 5′ terminus.

RNA or RNA molecule: As used herein, the term “RNA” or “RNA molecule” or “ribonucleic acid molecule” refers to a polymer of ribonucleotides; the term “DNA” or “DNA molecule” or “deoxyribonucleic acid molecule” refers to a polymer of deoxyribonucleotides. DNA and RNA can be synthesized naturally, e.g., by DNA replication and transcription of DNA, respectively; or be chemically synthesized. DNA and RNA can be single-stranded (i.e., ssRNA or ssDNA, respectively) or multi-stranded (e.g., double stranded, i.e., dsRNA and dsDNA, respectively). The term “mRNA” or “messenger RNA”, as used herein, refers to a single stranded RNA that encodes the amino acid sequence of one or more polypeptide chains.

RNA interfering or RNAi: As used herein, the term “RNA interfering” or “RNAi” refers to a sequence specific regulatory mechanism mediated by RNA molecules which results in the inhibition or interfering or “silencing” of the expression of a corresponding protein-coding gene. RNAi has been observed in many types of organisms, including plants, animals and fungi. RNAi occurs in cells naturally to remove foreign RNAs (e.g., viral RNAs). Natural RNAi proceeds via fragments cleaved from free dsRNA which direct the degradative mechanism to other similar RNA sequences. RNAi is controlled by the RNA-induced silencing complex (RISC) and is initiated by short/small dsRNA molecules in cell cytoplasm, where they interact with the catalytic RISC component argonaute. The dsRNA molecules can be introduced into cells exogenously. Exogenous dsRNA. initiates RNAi by activating the ribonuclease protein Dicer, which binds and cleaves dsRNAs to produce double-stranded fragments of 21-25 base pairs with a few unpaired overhang bases on each end. These short double stranded fragments are called small interfering RNAs (siRNAs).

Sample: As used herein, the term “sample” or “biological sample” refers to a subset of its tissues, cells or component parts (e.g. body fluids, including but not limited to blood, mucus, lymphatic fluid, synovial fluid, cerebrospinal fluid, saliva, amniotic fluid, amniotic cord blood, urine, vaginal fluid and semen). A sample further may include a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, organs. A sample further refers to a medium, such as a nutrient broth or gel, which may contain cellular components, such as proteins or nucleic acid molecule.

Self-complementary viral particle: As used herein, a “self-complementary viral particle” is a particle included of at least two components, a protein capsid and a polynucleotide sequence encoding a self-complementary genome enclosed within the capsid.

Sense Strand: As used herein, the term “the sense strand” or “the second strand” or “the passenger strand” of a siRNA molecule refers to a strand that is complementary to the antisense strand or first strand. The antisense and sense strands of a siRNA molecule are hybridized to form a duplex structure. As used herein, a “siRNA duplex” includes a siRNA strand having sufficient complementarity to a section of about 10-50 nucleotides of the mRNA of the gene targeted for silencing and a siRNA strand having sufficient complementarity to form a duplex with the other siRNA strand.

Short interfering RATA or siRNA: As used herein, the terms “short interfering RNA,” “small interfering RNA” or “siRNA” refer to an RNA molecule (or RNA analog) comprising between about 5-60 nucleotides (or nucleotide analogs) which is capable of directing or mediating RNAi. In certain embodiments, a siRNA molecule includes between about 15-30 nucleotides or nucleotide analogs, such as between about 16-25 nucleotides (or nucleotide analogs), between about 18-23 nucleotides (or nucleotide analogs), between about 19-22 nucleotides (or nucleotide analogs) (e.g., 19, 20, 21 or 22 nucleotides or nucleotide analogs), between about 19-25 nucleotides (or nucleotide analogs), and between about 19-24 nucleotides (or nucleotide analogs). The term “short” siRNA refers to a siRNA comprising 5-23 nucleotides, such as 21 nucleotides (or nucleotide analogs), for example, 19, 20, 21 or 22 nucleotides. The term “long” siRNA refers to a siRNA comprising 24-60 nucleotides, such as about 24-25 nucleotides, for example, 23, 24, 25 or 26 nucleotides. Short siRNAs may, in some instances, include fewer than 19 nucleotides, e.g., 16, 17 or 18 nucleotides, or as few as 5 nucleotides, provided that the shorter siRNA retains the ability to mediate RNAi. Likewise, long siRNAs may, in some instances, include more than 26 nucleotides, e.g., 27, 28, 29, 30, 35, 40, 45, 50, 55, or even 60 nucleotides, provided that the longer siRNA retains the ability to mediate RNAi or translational repression absent further processing, e.g., enzymatic processing, to a short siRNA. siRNAs can be single stranded RNA molecules (ss-siRNAs) or double stranded RNA molecules (ds-siRNAs) comprising a sense strand and an antisense strand which hybridized to form a duplex structure called siRNA duplex.

Signal Sequences: As used herein, the phrase “signal sequences” refers to a sequence which can direct the transport or localization of a protein.

Single unit dose: As used herein, a “single unit dose” is a dose of any therapeutic administered in one dose/at one time/single route/single point of contact, i.e., single administration event. In certain embodiments, a single unit dose is provided as a discrete dosage form (e.g., a tablet, capsule, patch, loaded syringe, vial, etc.).

Similarity: As used herein, the term “similarity” refers to the overall relatedness between polymeric molecules, e.g. between polynucleotide molecules (e.g. DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art.

Split dose: As used herein, a “split dose” is the division of single unit dose or total daily dose into two or more doses.

Stable: As used herein “stable” refers to a compound that is sufficiently robust to survive isolation to a useful degree of purity from a reaction mixture, and in certain embodiments, capable of formulation into an efficacious therapeutic agent.

Stabilized: As used herein, the term “stabilize”, “stabilized,” “stabilized region” means to make or become stable.

Subject: As used herein, the term “subject” or “patient” refers to any organism to which a composition in accordance with the present disclosure may be administered, e.g., for experimental, diagnostic, prophylactic, and/or therapeutic purposes. Typical subjects include animals (e.g., mammals such as mice, rats, rabbits, non-human primates, and humans) and/or plants.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Substantially equal: As used herein as it relates to time differences between doses, the term means plus/minus

Substantially simultaneously: As used herein and as it relates to plurality of doses, the term means within 2 seconds.

Suffering from: An individual who is “suffering from” a disease, disorder, and/or condition has been diagnosed with or displays one or more symptoms of a disease, disorder, and/or condition.

Susceptible to: An individual who is “susceptible to” a disease, disorder, and/or condition has not been diagnosed with and/or may not exhibit symptoms of the disease, disorder, and/or condition but harbors a propensity to develop a disease or its symptoms. In certain embodiments, an individual who is susceptible to a disease, disorder, and/or condition (for example, cancer) may be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein and/or nucleic acid associated with the disease, disorder, and/or condition; (4) habits and/or lifestyles associated with development of the disease, disorder, and/or condition; (5) a family history of the disease, disorder, and/or condition; and (6) exposure to and/or infection with a microbe associated with development of the disease, disorder, and/or condition. In certain embodiments, an individual who is susceptible to a disease, disorder, and/or condition will develop the disease, disorder, and/or condition. In certain embodiments, an individual who is susceptible to a disease, disorder, and/or condition will not develop the disease, disorder, and/or condition.

Sustained release: As used herein, the term “sustained release” refers to a pharmaceutical composition or compound release profile that conforms to a release rate over a specific period of time.

Synthetic: The term “synthetic” means produced, prepared, and/or manufactured by the hand of man. Synthesis of polynucleotides or polypeptides or other molecules of the present disclosure may be chemical or enzymatic.

Targeting: As used herein, “targeting” means the process of design and selection of nucleic acid sequence that will hybridize to a target nucleic acid and induce a desired effect.

Targeted Cells: As used herein, “targeted cells” refers to any one or more cells of interest. The cells may be found in vitro, in vivo, in situ or in the tissue or organ of an organism. The organism may be an animal, such as a mammal, a human, or a human patient.

Terminal region: As used herein, the term “terminal region” refers to a region on the 5′ or 3′ end of a region of linked nucleosides or amino acids (polynucleotide or polypeptide, respectively).

Terminally optimized: The term “terminally optimized” when referring to nucleic acids means the terminal regions of the nucleic acid are improved in some way, e.g., codon optimized, over the native or wild type terminal regions.

Therapeutic Agent: The term “therapeutic agent” refers to any agent that, when administered to a subject, has a therapeutic, diagnostic, and/or prophylactic effect and/or elicits a desired biological and/or pharmacological effect.

Therapeutically effective amount: As used herein, the term “therapeutically effective amount” means an amount of an agent to be delivered (e.g., nucleic acid, drug, therapeutic aunt, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition. In certain embodiments, a therapeutically effective amount is provided in a single dose. In certain embodiments, a therapeutically effective amount is administered in a dosage regimen comprising a plurality of doses. Those skilled in the art will appreciate that in certain embodiments, a unit dosage form may be considered to include a therapeutically effective amount of a particular agent or entity if it includes an amount that is effective when administered as part of such a dosage regimen.

Therapeutically effective outcome: As used herein, the term “therapeutically effective outcome” means an outcome that is sufficient in a subject suffering from or susceptible to an infection, disease, disorder, and/or condition, to treat, improve symptoms of, diagnose, prevent, and/or delay the onset of the infection, disease, disorder, and/or condition.

Total daily dose: As used herein, a “total daily dose” is an amount given or prescribed in 24-hour period. It may be administered as a single unit dose.

Transfection: As used herein, the term “transfection” refers to methods to introduce exogenous nucleic acids into a cell. Methods of transfection include, but are not limited to, chemical methods, physical treatments and cationic lipids or mixtures, The list of agents that can be transfected into a cell is large and includes, but is not limited to, siRNA, sense and/or anti-sense sequences, DNA encoding one or more genes and organized into an expression plasmid, proteins, protein fragments, and more.

Treating,: As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Unmodified: As used herein, “unmodified” refers to any substance, compound or molecule prior to being changed in any way. Unmodified may, but does not always, refer to the wild type or native form of a biomolecule. Molecules may undergo a series of modifications whereby each modified molecule may serve as the “unmodified” starting molecule for a subsequent modification,

Vector: As used herein, a “vector” is any molecule or moiety which transports, transduces or otherwise acts as a carrier of a heterologous molecule. Vectors of the present disclosure may be produced recombinantly and may be based on and/or may include adeno-associated virus (AAV) parent or reference sequence. Such parent or reference AAV sequences may serve as an original, second, third or subsequent sequence for engineering vectors. In non-limiting examples, such parent or reference AAV sequences may include any one or more of the following sequences: a polynucleotide sequence encoding a polypeptide or multi-polypeptide, which sequence may be wild-type or modified from wild-type and which sequence may encode full-length or partial sequence of a protein, protein domain, or one or more subunits of a protein; a polynucleotide comprising a modulatory or regulatory nucleic acid which sequence may be wild-type or modified from wild-type; and a transgene that may or may not be modified from wild-type sequence. These AAV sequences may serve as either the “donor” sequence of one or more codons (at the nucleic acid level) amino acids (at the polypeptide level) or “acceptor” sequences of one or more codons (at the nucleic acid level) or amino acids (at the polypeptide level).

Viral genome: As used herein, a “viral genome” or “vector genome” or “viral vector” refers to the nucleic acid sequence(s) encapsulated in an AAV particle, Viral genomes comprise at least one payload region encoding polypeptides or fragments thereof.

### EXAMPLES

### Example 1

### Dose Optimization Study 1

i. Study Design

The primary objective of this study was to evaluate delivery parameters to optimize distribution of an AAV 1 packaged AAV1-miRNA expression vector comprising an ITR to ITR sequence, VOYHT1, (hereinafter referred to as AAV1-VOYHT1 or VY-HTT01; SEQ ID NO for VOYHT: 1352) within the striatum, cortex and thalamus of rhesus macaques, and to provide a basis for establishing future dosing parameters and for extrapolation to a clinical dosing paradigm. A secondary objective was to conduct a limited safety and tolerability assessment of delivery parameters.

The rhesus macaque (Macaca mulatto) was selected as the test system due to its established usefulness and acceptance as a model for pharmacological and toxicological studies, especially when using gene therapy delivery to the central nervous system (CNS). The more completely understood mapping of rhesus genome, relative to other non-human primates (NHPs), is particularly relevant for assessment of RNA interference products, The large brain volume and anatomical structure were also important factors taken into consideration when choosing this species to address the study objectives.

This study involved the screening of 34 animals to obtain 18 for dosing and 2 alternates. The 18 animals were assigned to 6 treatment groups as summarized in Table 40. Bilateral intraparenchymal infusion into the putamen and thalamus was chosen to maximize brain distribution via axonal transport to cortical areas. Also, putamen and thalamus were preferred infusion sites because putamen and thalamus in early HD human patients are 4-5 times larger than in rhesus, and severe atrophy of the caudate nucleus would prevent direct infusion into the caudate.

TABLE 40
Study design

| | | | | | | Total |
|---|---|---|---|---|---|---|
| | | Number of | Volume (μL/side) | | Dosing Titer | dose |
| Group | Description | animals | Putamen | Thalamus | (vg/μl) | (vg) |
| Group A1 | Low Vol; High Conc. | 3 | 50 | 75 | 2.7e12 | 6.8e11 |
| Group A2 | Mid Vol; High Conc. | 3 | 100 | 150 | 2.7e12 | 1.4e12 |
| Group A3 | High Vol; High Conc. | 3 | 150 | 250 | 2.7e12 | 2.2e12 |
| Group A4 | Mid Vol; Mid Conc. | 3 | 100 | 150 | 9.0e11 | 4.5e11 |
| Group A5 | Mid Vol; Lower Conc. | 3 | 100 | 150 | 2.7e11 | 1.4e11 |
| Group A6 | Vehicle Control | 3 | Left: 100 | Left: 150 | N/A | N/A |
| | | | Right: 150 | Right: 250 | | |

The calculated human equivalent dose corresponding to each group in Table 40 is presented in Table 41.

TABLE 41
Human equivalent dose

| | | Human Equivalent Dose | | |
|---|---|---|---|---|
| | | | | Total |
| | | Putamen | Thalamus | dose |
| Group | Description | (μL/side) | (μL/side) | (vg) |
| Group A1 | Low Vol; High Conc. | 175 | 450 | 3.4e12 |
| Group A2 | Mid Vol; High Conc. | 350 | 900 | 6.8e12 |
| Group A3 | High Vol; High Conc. | 525 | 1500 | 1.1e13 |
| Group A4 | Mid Vol; Mid Conc. | 350 | 900 | 2.3e12 |
| Group A5 | Mid Vol; Lower Conc. | 350 | 900 | 6.8e11 |
| Group A6 | Vehicle Control | Left: 350 | Left: 900 | N/A |
| | | Right: 525 | Right: 1500 | |

Each animal received bilateral intracranial infusion of the test article containing AAV1-VOYHT1 or a vehicle control into the putamen and thalamus using magnetic resonance imaging (MRI)-guided convection-enhanced delivery (CED). Animals were euthanized 5 weeks (Day 36±3) after dosing, and tissues were collected for post-mortem analysis.

ii. Animal Care and Sample Collection

Thirty-four (N=34) healthy adult male or female rhesus macaques (4-10 years old) were selected for prescreening. Animals weighed 4-10 kg. Animals were acclimated for a minimum of 2 weeks after clearance from Centers for Disease Control and Prevention (CDC) quarantine, Animals had a pre-project blood sample collected for screening of anti-AAV1 neutralizing antibodies (nAb) titers. Eighteen (N=18) animals with anti-AAV1 NAb serum titers ≤1:16 were selected, weighed, and randomized into the study groups for dosing as indicated in Table 40. An additional 2 animals were selected as alternate study animals. Animals were maintained on Harlan 20% Primate Diet with ad libitum access to water. Samples of water were routinely analyzed for specified microorganisms and environmental contaminants. Environmental controls for the animal room were set to maintain 70±6° F., a minimum of 10 air changes/hour, and a 12-hour light/12-hour dark cycle. Cage-side monitoring were performed twice daily, and food consumption assessment was performed once daily. Body weight was measured once per week. Animals were housed in individual cages throughout the study.

Blood samples were collected for clinical pathology evaluation and neutralizing antibody (nAb) analysis at Pre-dose (i.e., 7 days prior to the initiation of dosing of the first animal receiving AAV1VOYHT1 infusion), Day 15±2, and immediately prior to necropsy on Day 36±3. The clinical pathology evaluation included hematology (CBC), serum clinical chemistry (Chem), and coagulation (Coag) analysis. Cerebrospinal fluid (CSF) samples were collected for nAb analysis from the cervical region prior to dosing (Day 1). and immediately prior to necropsy on Day 36±3. Following necropsy, the brain, spinal cord, dorsal root ganglia, and major organs were collected and then fresh frozen or 4% paraformaldehyde (PFA) post-fixed by immersion.

iii. Test Article Preparation and Dosing Procedures

The test article used in the study contained AAV1-VOYHT1 gene transfer vector (2.7e12 vg/mL) formulated in aqueous solution containing 192 mM sodium chloride, 10 mM sodium phosphate. 2 mM potassium phosphate, 2.7 mM potassium chloride, and 0.001% Poloxamer 188 (Pluronic® F-68). The vehicle control contained the formulation buffer only. The samples were stored at −60° C. or below and were thawed to and maintained at 2-8° C. on day of dosing. ProHance® (Bracco Diagnostics, Inc), i.e. gadoteridol, was added at a 1:250 ratio (1 μL of ProHance per 250 μL of test article or control) and carefully mixed by inverting tubes prior to loading into the infusion system. The dosing solution contained the test article or control and a 2 mM concentration of gadoteridol. Dilution of the dosing solutions are summarized in Table 42. “N/A” indicates data not applicable.

TABLE 42
Dilutions of dosing solutions

| | Final vector | Vector | | | Final |
|---|---|---|---|---|---|
| | concentration | stock | Diluent | ProHance | Volume |
| Group | (vg/mL) | (μL) | (μL) | (μL) | (μL) |
| A1, A2, A3 | 2.7e12 | 2250 | 0 | 9 | 2259 |
| A4 | 9.0e11 | 700 | 1392 | 8.4 | 2100 |
| A5 | 2.7e11 | 210 | 1882 | 8.4 | 2100 |
| A6 | N/A | N/A | 15000 | 60 | 15060 |

Immediately prior to surgery, each animal was anesthetized with intramuscular (IM) Ketamine (10 mg/kg) and IM dexmedetomidine (15 μg/kg), weighed, intubated, and maintained on 1-5% Isoflurane. The head was secured onto a stereotaxic frame and the overlying skin prepared for neurosurgical implantation procedures. Using aseptic techniques, the wound site was opened in anatomical layers to expose the skull. A bilateral craniotomy was performed at entry sites located above the frontal and/or parietal lobe on each side. A skull mounted cannula guide ball array was temporarily secured to the skull over each burr hole using titanium screws. Immediately after surgery to implant cannula guides, the animal was transferred to the MRI suite. MR imaging was used to align cannula guides with putamen and thalamus targets ipsilateral to each cannula guide. Test article or control was administered with repeated MR imaging to visually monitor infusions within the brain as specified in Table 40 above. Each animal received up to 2 infusions (sites) of test article or control using convection enhanced delivery (CED) in each putamen and thalamus. An adjustable tip 16G cannula (MRI Interventions Inc.) was guided into each target site through the skull mounted cannula arrays. The cannula was connected to a syringe mounted on a syringe pump (Harvard Apparatus). Dose volumes (50-400 μL per hemisphere) were deposited into each putamen or thalamnus using ascending infusion rates (up to 10 μL/minute), Serial MRI scans were acquired to monitor infusate distribution within each target site and provide real-time monitoring of the dosing. In some cases, cannula was advanced deeper into the putamen or thalamus during the infusion to maximize infusate distribution within the putamen or thalamus. Immediately after the MRI CED dosing procedure, the animal was transferred back to the operating room, the cannula guide system was explanted, and the wound site was closed in anatomical layers with absorbable vicryl suture and using a simple interrupted suturing pattern. Pre- and post-operative medications included buprenorphine (0.03 mg/kg, IM, b.i.d.), carprofen (2.2 mg/kg SQ, b.i.d.), ketoprofen (2 mg/kg, IM, s.i.d.), and cefazolin (100 mg IV, pre- and post-surgery, followed by 25 mg/kg, IM, b.i.d) or ceftriaxone (50 mg/kg, IM, s.i.d.), Animals were monitored for full recovery from anesthesia and returned to their home cages.

iv. HTT Knockdown and Vector Genome (VG Measurement in punches from NHP Striatum, Cortex, and Thalamus across Different Infusion Volumes

This analysis was designed to evaluate the impact of different infusion volumes on vector distribution and coverage. Selected brain slabs containing the motor and somatosensoty cortex and anterior putamen from Groups A1 (low vol), A2 (med vol), A3 (high vol), and A6 (control) were used to collect 2 mm punches. Six cortex, 8 putamen, 2 caudate, and 5 thalamus punches were collected from each side of the brain (42 total per animal), with a total number of 504 punches collected from all four groups. Samples were homogenized in QuantiGene® homogenization buffer and subjected to protease K digest. Cleared cell lysates were generated and processed for both HTT mRNA measurement using a branched DNA (bDNA) assay and vector genome (VG) measurement using droplet digital PCR (ddPCR) after an additional DNA purification step (Qtagen, catalog-469506). The bDNA assay was carried out according to the QuantiGene® Plex Assay (Thermaisher Scientific) protocol using a probe set specifically for rhesus HTT. Cell lysate was assayed in duplicates. HTT mRNA level was normalized to the geometric mean of three rhesus housekeeping genes, i.e., AARS, TBP, and XPNPEP1. Results were calibrated to the normalized mean of the vehicle group and presented as: mean of relative remaining HTT mRNA (%)±standard deviation (stdev). For ddPCR, whole cell DNA was prepared from same tissue homogenate used in the bDNA assay. The level of vector genome detected with probe set, CBA Promoter, was normalized to a Host probe set (RNase P). All samples were blinded during the analysis.

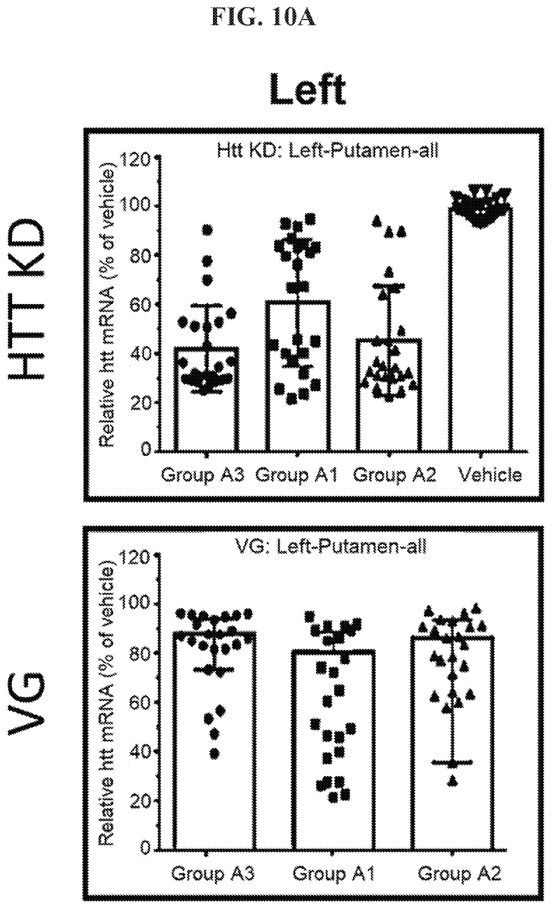

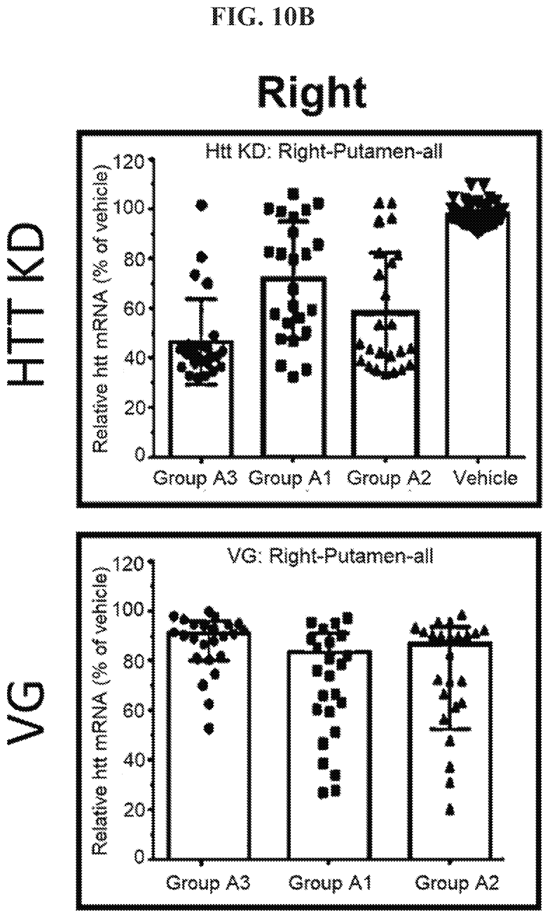

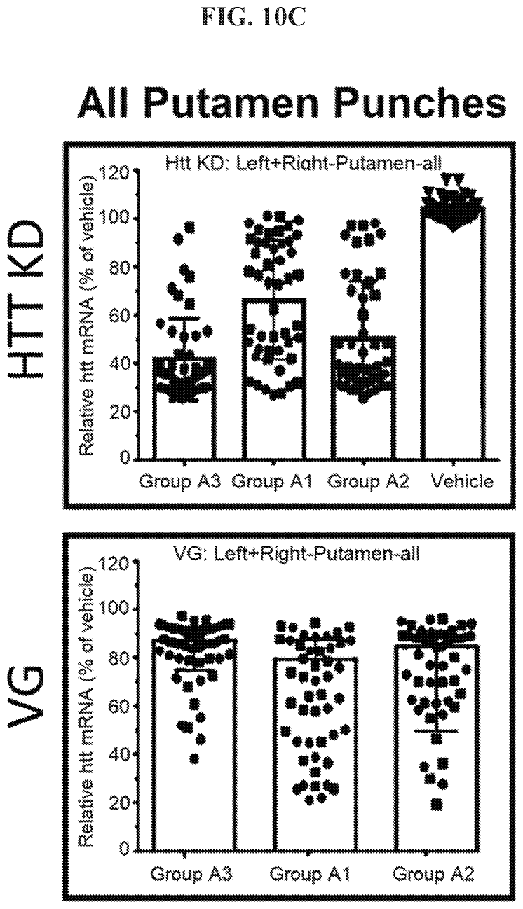

In the putamen, all groups showed HTT mRNA knockdown, with 63%, 48% and 39% HTT mRNA remaining relative to vehicle for Groups A1, A2, and A3, respectively (see FIG. 10A-C). Of the 16 punches per animal (total 3 animals per group), an average of 52%, 79%, and 92% of the punches for Group A1, A2 and A3 respectively, reached at least 30% HTT mRNA knockdown. HTT mRNA levels in putamen from each AAV1-VOYHT1-treated group averaged per animal after normalization to the vehicle control group are presented in Table 43.

TABLE 43
HTT mRNA knockdown in putamen punches across
infusion volumes averaged per animal

| | | | HTT mRNA |
|---|---|---|---|
| | | Number | relative to vehicle |
| Group | Description | of animals | (% ± stdev) |
| Group A1 | Low Vol; High Conc. | 3 | 63 ± 12 |
| Group A2 | Mid Vol; High Conc. | 3 | 48 ± 7 |
| Group A3 | High Vol; High Conc. | 3 | 39 ± 8 |
| Group A6 | Vehicle Control | 3 | 100 ± 1 |

When VG copies were analyzed in all putamen punches sampled from each of the three groups, differential VG distributions were observed across different vector infusion volumes. The highest and most stable VG distribution pattern was observed in Group A3, followed by Group A2 and Group A1 (see FIG. 10A-C). This differential vector distribution pattern was observed in left and right hemispheres. VG levels tracked with putamen HTT knockdown, with Group A3 possessing both the highest VG representation and the greatest HTT mRNA knockdown. For VG levels, the number of VG copies detected in putamen punches from each group averaged per animal is presented in Table 44.

TABLE 44
VG copies in putamen punches across
infusion volumes averaged per animal

| | | Sample | VG copies/cell |
|---|---|---|---|
| Group | Description | size | (mean ± stdev) |
| Group A1 | Low Vol; High Conc. | 3 | 327.2 ± 191.2 |
| Group A2 | Mid Vol; High Conc. | 3 | 527.5 ± 207.0 |
| Group A3 | High Vol; High Conc. | 3 | 710.5 ± 163.8 |
| Group A6 | Vehicle Control | 3 | 0.2 ± 0.2 |

A Grubbs' test (Q=0.1%) was applied for removal of outliers and the VG copies/cell recalculated. Following this post-hoc statistical analysis, VG copies in putamen punches per animal were unchanged for groups A1 and A3, but for Group A2, VG copies/cell was recalculated to 489.7±204.0.

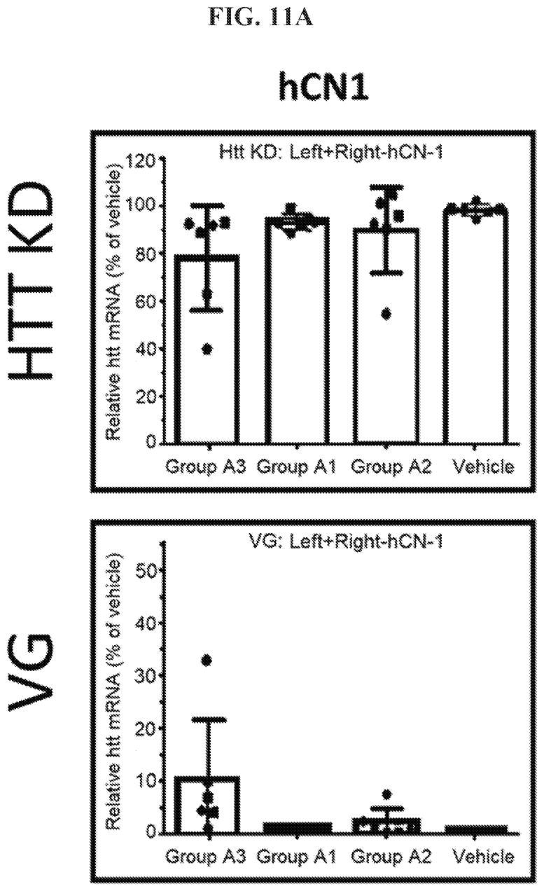

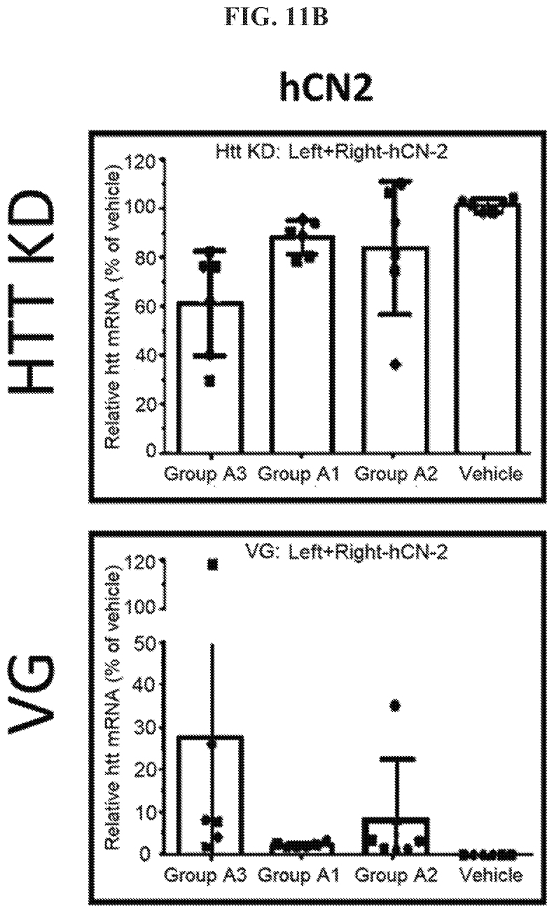

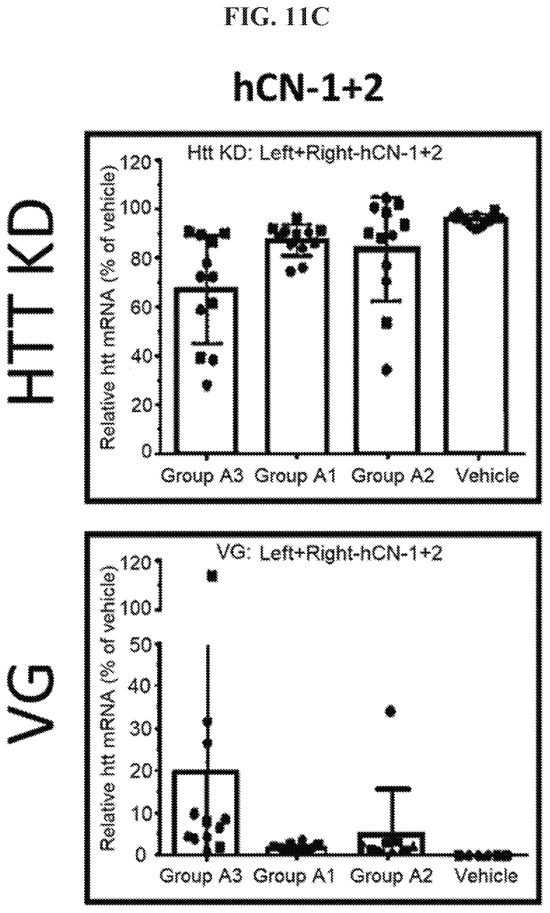

In the caudate, Group A3 showed the greatest HTT mRNA knockdown with 70% HTT mRNA remaining relative to vehicle (see FIG. 11A-C). Group A1 and Group A2 showed 91% and 87% HTT mRNA remaining relative to vehicle, respectively. VG levels correlated with HTT mRNA knockdown (see FIG. 11A-C). HTT mRNA levels in caudate from each AAV1-VOYHT1-treated group averaged per animal after normalization to the vehicle control group are presented in Table 45.

TABLE 45
HTT mRNA knockdown in caudate punches across
infusion volumes averaged per animal

| | | | HTT mRNA |
|---|---|---|---|
| | | Number | relative to vehicle |
| Group | Description | of animals | (% ± stdev) |
| Group A1 | Low Vol; High Conc. | 3 | 91 ± 2 |
| Group A2 | Mid Vol; High Conc. | 3 | 87 ± 18 |
| Group A3 | High Vol; High Conc. | 3 | 70 ± 23 |
| Group A6 | Vehicle Control | 3 | 100 ± 1 |

When VG copies were analyzed in all caudate punches sampled from each of the three groups, VG levels tracked with HTT mRNA knockdown (see FIG. 11A-C). Hence, Group A3 exhibited the highest VG representation and the greatest HTT mRNA knockdown. For VG levels, the number of VG copies detected in caudate punches from each group averaged per animal is presented in Table 46,

TABLE 46
VG copies in candate punches across
infusion volumes averaged per animal

| | | Sample | VG copies/cell |
|---|---|---|---|
| Group | Description | size | (mean ± stdev) |
| Group A1 | Low Vol; High Conc. | 3 | 1.6 ± 0.3 |
| Group A2 | Mid Vol; High Conc. | 3 | 4.9 ± 5.4 |
| Group A3 | High Vol; High Conc. | 3 | 18.8 ± 24.2 |
| Group A6 | Vehicle Control | 3 | 00.0 ± 0.1 |

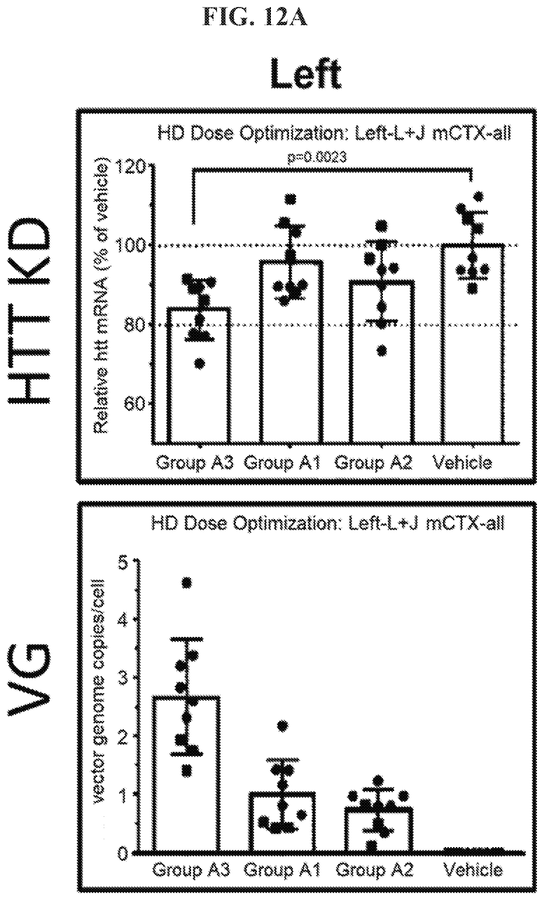

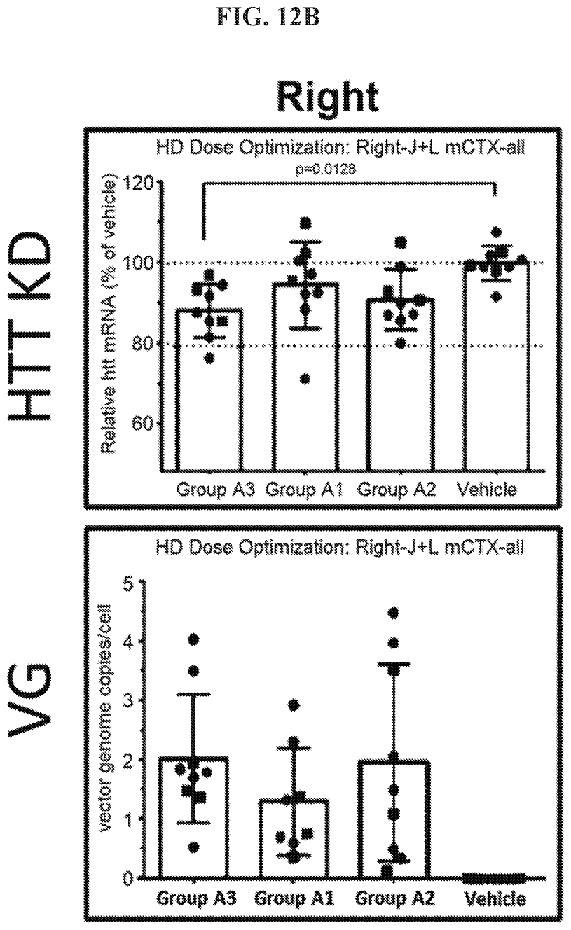

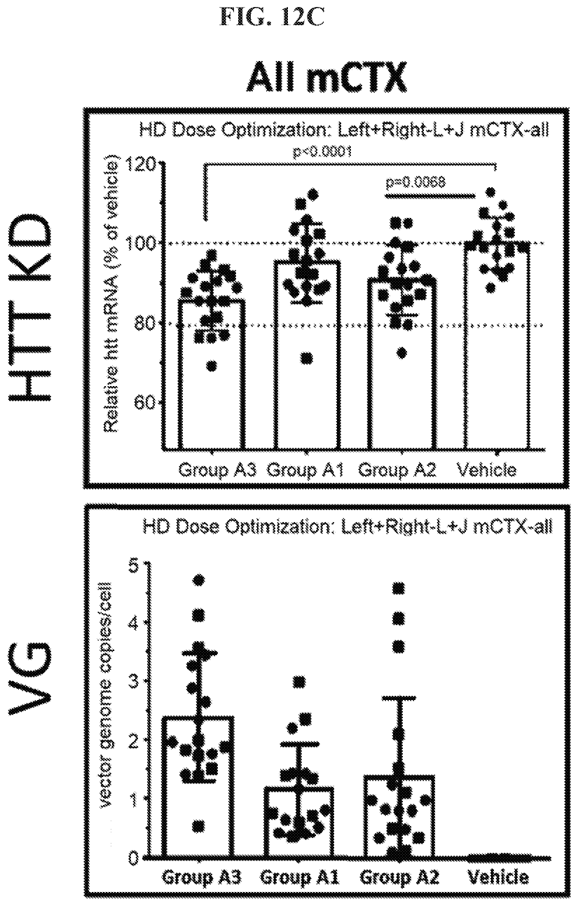

When a Grubbs' test (Q=0.1%) was applied to remove outliers, the average number of VG copies/cell detected in caudate punches remained unchanged for Group A1, but was re-quantified as 1.8±0.5 and 10.7±10.3 for Groups A2 and A3, respectively. Punches were analyzed from three cortical areas: motor cortex (mCTX), somatosensory cortex (ssCTX), and temporal cortex (tCTX) in the mCTX, significant HTT knockdown was observed for Groups A3 and A2, with greater knockdown in Group A3 than Group A2, resulting in 86% and 91% HTT snRNA remaining relative to vehicle, respectively (see FIG. 12A-C). HTT mRNA levels in mCTX from each AAV1-VOYHT1-treated group averaged per animal after normalization to the vehicle control group are presented in Table 47.

TABLE 47
HTT mRNA knockdown in mCTX punches across
infusion volumes averaged per animal

| | | | HTT mRNA |
|---|---|---|---|
| | | Number | relative to vehicle |
| Group | Description | of animals | (% ± stdev) |
| Group A1 | Low Vol; High Conc. | 3 | 95 ± 1 |
| Group A2 | Mid Vol; High Conc. | 3 | 91 ± 3 |
| Group A3 | High Vol; High Conc. | 3 | 86 ± 6 |
| Group A6 | Vehicle Control | 3 | 100 ± 3 |

When VG copies were analyzed in all mCTX punches sampled from each of the three groups. VG levels were lower in mCTX than in the putamen in all groups, with Group A3 showing the highest VG representation (see FIG. 12A-C). VG variability was seen between left and right sides of the mCTX. For VG levels, the number of VG copies detected in mCTX punches from each group averaged per animal is presented in Table 48. VG copies were below the quantification limit (BLQ) for Group A6 (vehicle control).

TABLE 48
VG copies in mCTX punches across infusion
volumes averaged per animal

| | | Sample | VG copies/cell |
|---|---|---|---|
| Group | Description | size | (mean ± stdev) |
| Group A1 | Low Vol; High Conc. | 3 | 1.36 ± 0.8 |
| Group A2 | Mid Vol; High Conc. | 3 | 1.34 ± 1.03 |
| Group A3 | High Vol; High Conc. | 3 | 2.35 ± 0.3 |
| Group A6 | Vehicle Control | 3 | BLQ |

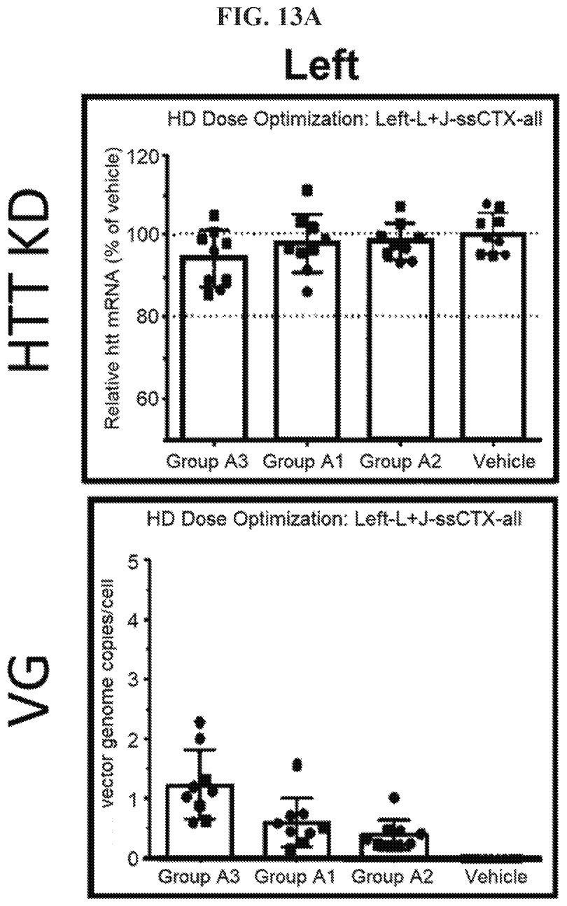

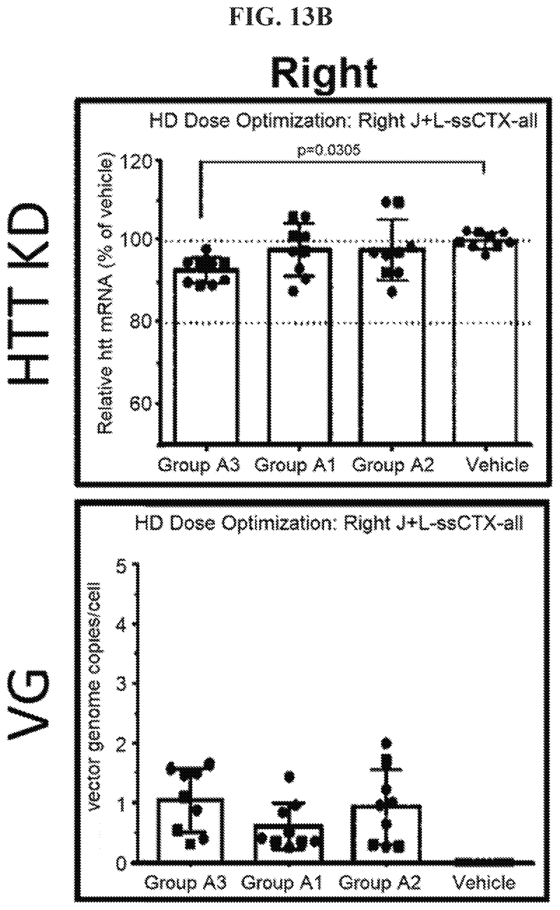

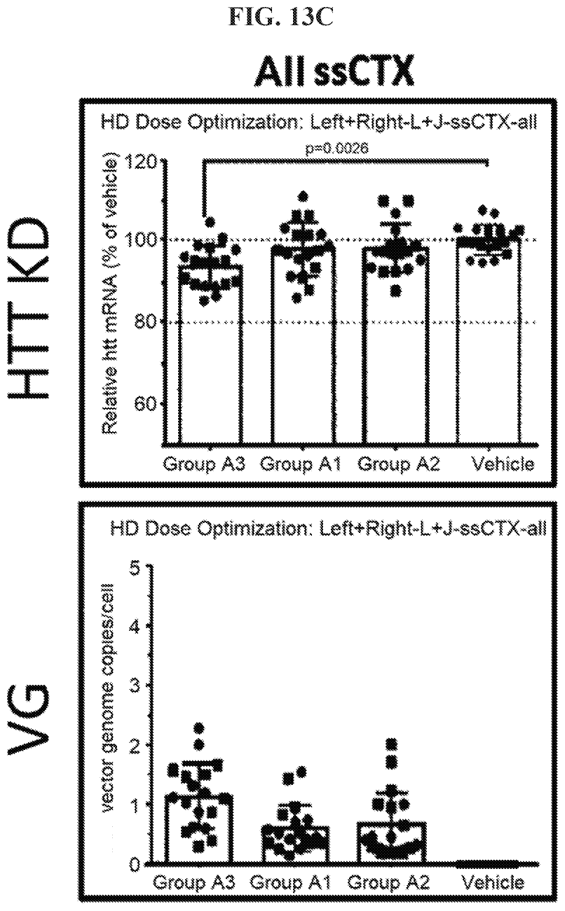

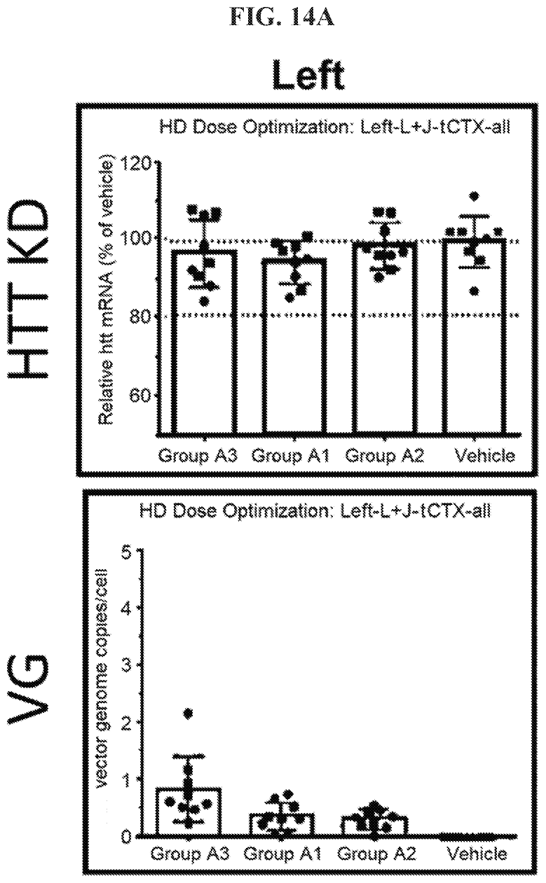

In the ssCTX, HTT knockdown was seen in somatosensory cortex of Group A3 only, where 93% of HTT mRNA remained relative to vehicle (see FIG. 13A-C), HTT mRNA levels in ssCTX from each AAV1-VOYHT1 -treated group averaged per animal after normalization to the vehicle control group are presented in Table 49.

TABLE 49
HTT mRNA knockdown in ssCTX punches across
infusion volumes averaged per animal

| | | | HTT mRNA |
|---|---|---|---|
| | | Number | relative to vehicle |
| Group | Description | of animals | (% ± stdev) |
| Group A1 | Low Vol; High Conc. | 3 | 96 ± 4 |
| Group A2 | Mid Vol; High Conc. | 3 | 97 ± 3 |
| Group A3 | High Vol; High Conc. | 3 | 94 ± 6 |
| Group A6 | Vehicle Control | 3 | 100 ± 2 |

When VG copies were analyzed in all ssCTX punches sampled from each of the the three groups, VG levels were detected at levels lower than observed in mCTX in all groups, and Group A3 had a relatively higher VG representation than Group A1 and Group A2 (see FIG. 13A-C). For VG levels, the average number of VG copies detected in mCTX punches from each group averaged per animal is presented in Table 50. VG copies were below the quantification limit (BLQ) for Group A6 (vehicle control).

TABLE 50
VG copies in ssCTX punches across infusion
volumes averaged per animal

| | | Sample | VG copies/cell |
|---|---|---|---|
| Group | Description | size | (mean ± stdev) |
| Group A1 | Low Vol; High Conc. | 3 | 0.61 ± 0.3 |
| Group A2 | Mid Vol; High Conc. | 3 | 0.67 ± 0.3 |
| Group A3 | High Vol; High Conc. | 3 | 1.13 ± 0.3 |
| Group A6 | Vehicle Control | 3 | BLQ |

Combined mCTX and ssCTX samples were also included in cortical punch analyses. When mCTX and ssCTX samples were combined, HTT mRNA remaining relative to vehicle was 95±3% (mean±stdev), 94±5%, and 90±5% for Group A1, Group A2 and Group A3, respectively. HTT mRNA remaining for the vehicle control Group A6 was 100±2% relative to control, Thus. HTT mRNA knockdown was about 5% for Group A1, 6% for Group A2, and 10% for Group A3, HTT mRNA levels in combined mCTX and ssCTX samples from each AAV1-VOYHT1-treated group averaged per animal after normalization to the vehicle control group are also presented in Table 51.

TABLE 51
HTT mRNA knockdown in combined mCTX and ssCTX punches
across infusion volumes averaged per animal

| | | | HTT mRNA |
|---|---|---|---|
| | | Number | relative to vehicle |
| Group | Description | of animals | (% ± stdev) |
| Group A1 | Low Vol; High Conc. | 3 | 95 ± 3 |
| Group A2 | Mid Vol, High Conc. | 3 | 94 ± 5 |
| Group A3 | High Vol; High Conc. | 3 | 90 ± 5 |
| Group A6 | Vehicle Control | 3 | 100 ± 2 |

For VG levels in combined mCTX and ssCTX punches, Group A3 showed 1.74±0.3 VG copies/cell (averaged per animal), a higher VG representation than observed in punches from Group A2 and Group A1, which contained 1.01±0.7 and 0.99±0.4 VG copies/cell, respectively. VG copies were below the quantification limit (BLQ) for Group A6 (vehicle control). For VG levels, the average number of VG copies detected in combined mCTX and ssCTX punches from each group averaged per animal is presented in Table 52.

TABLE 52
VG copies in combined mCTX and ssCTX punches
across infusion volumes averaged per animal

| | | Number | VG copies/cell |
|---|---|---|---|
| Group | Description | of animals | (mean ± stdev) |
| Group A1 | Low Vol; High Conc. | 3 | 0.99 ± 0.4 |
| Group A2 | Mid Vol; High Conc. | 3 | 1.01 ± 0.7 |
| Group A3 | High Vol; High Conc. | 3 | 1.74 ± 0.3 |
| Group A6 | Vehicle Control | 3 | BLQ |

Together, for mCTX and ssCTX combined samples, increasing infusion volume tracked with enhanced HTT knockdown and higher VG representation.

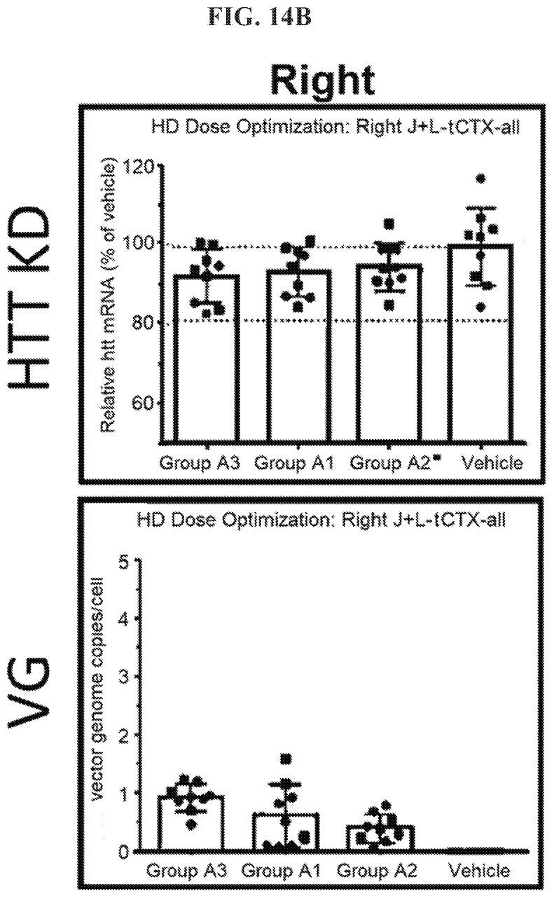

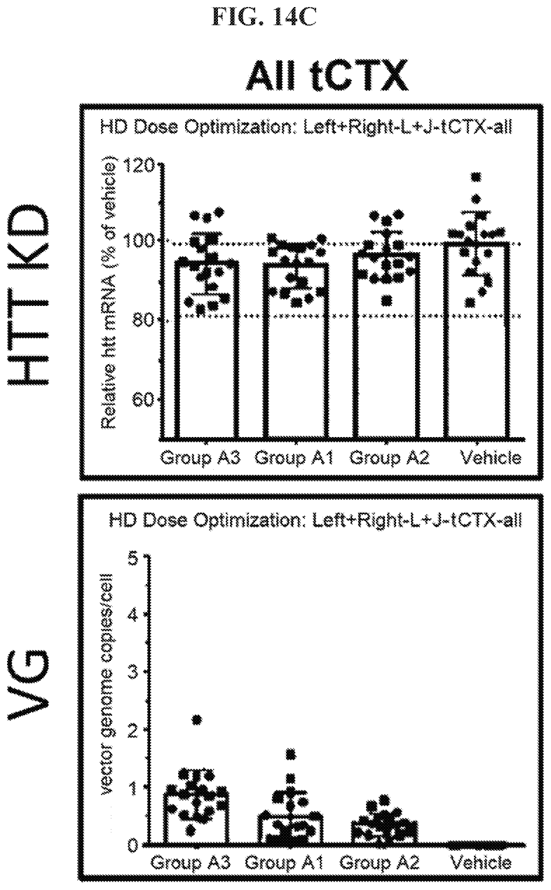

In the tCTX, no statistically significant HTT KD was seen for any of the three groups when all tCTX punches were included as individual datapoints in analyses (see FIG. 14A-C). When VG copies were similarly analyzed in all tCTX punches sampled from each of the three groups, lower VG representation was detected in all groups relative to other cortical areas, but Group A3 had a relatively higher VG representation than Groups A1 and A2 (see FIG. 14A-C). VG copies were below the quantification limit (BLQ) for Group A6 (vehicle control).

When VG copies were averaged across tissue punches from tCTX per animal, an average vector genome level of 0.54±0.31 VG copies/cell was observed at the low volume, 0.39±0.05 VG copies/cell at the middle volume, and 0.96±0.03 VG copies/cell at the highest volume. VG copies were below the quantification limit (BLQ) for Group A6 (vehicle control). The average number of VG copies detected in tCTX punches from each group averaged per animal is presented in Table 53.

TABLE 53
VG copies in tCTX punches across infusion
volumes averaged per animal

| | | Number | VG copies/cell |
|---|---|---|---|
| Group | Description | of animals | (mean ± stdev) |
| Group A1 | Low Vol; High Conc. | 3 | 0.54 ± 0.31 |
| Group A2 | Mid Vol; High Conc. | 3 | 0.39 ± 0.05 |
| Group A3 | High Vol; High Conc. | 3 | 0.96 ± 0.03 |
| Group A6 | Vehicle Control | 3 | BLQ |

Together, the high-volume group (Group A3) exhibited a greater number of VG copies per cell versus all other groups (p<0.05). There was no difference in VG copies per cell between low- and middle-volume groups (Groups A1 and A2, respectively).

In sum, VG was consistently detected in cortex, with the highest representation in mCTX, followed by ssCTX. Variability was observed between punches, cortical areas, and left and right hemispheres. Relatively greater HTT mRNA knockdown was observed in mCTX as compared to ssCTX and tCTX. Among the groups, Group A3 exhibited the highest VG representation and greatest HTT mRNA knockdown. A relationship between increasing VG levels and enhanced HTT mRNA knockdown was observed in cortex, as it was in the putamen and caudate.

In the thalamus, all groups demonstrated HTT mRNA knockdown with 35%, 38% and 30% HTT remaining relative to vehicle for Groups A1, A2, and A3, respectively. HTT mRNA levels in the thalamus from AAV1-VOYHT1-treated groups after normalization to the vehicle control group are presented in Table 54.

TABLE 54
HTT mRNA knockdown in thalamus punches across
infusion volumes averaged per animal

| | | | HTT mRNA |
|---|---|---|---|
| | | Number | relative to vehicle |
| Group | Description | of animals | (% ± stdev) |
| Group A1 | Low Vol; High Conc. | 3 | 35 ± 10 |
| Group A2 | Mid Vol; High Conc. | 3 | 38 ± 6 |
| Group A3 | High Vol; High Conc. | 3 | 30 ± 1 |
| Group A6 | Vehicle Control | 3 | 100 ± 2 |

For VG levels, the average number of VG copies detected in thalamus punches from each group averaged per animal is presented in Table 55. The thalamus exhibited the greatest VG representation with the largest infusion volume. Thus, Group A3 had a greater VG representation than Group A1 and A2. VG copies were below the quantification limit (BLQ) for Group A6 (vehicle control).

TABLE 55
VG copies in thalamus punches across
infusion volumes averaged per animal

| | | Sample | VG copies/cell |
|---|---|---|---|
| Group | Description | size | (mean ± stdev) |
| Group A1 | Low Vol; High Conc. | 3 | 849.9 ± 112.6 |
| Group A2 | Mid Vol; High Conc. | 3 | 773.0 ± 655.3 |
| Group A3 | High Vol; High Conc. | 3 | 1136.5 ± 270.8 |
| Group A6 | Vehicle Control | 3 | BLQ |

Overall, these observations demonstrated that vector volume affects vector bio-distribution in vivo, Among the tested areas, all groups displayed substantial HTT mRNA knockdown in putamen, while in caudate Group A3 led to substantial HTT knockdown. In cortex, mCTX (Groups A3 and A2) and ssCTX (Group A3) showed statistically significant HTT snRNA knockdown, which corresponded to high vector distribution. All groups demonstrated HTT mRNA knockdown in the thalamus, where VG representation was highest among all regions sampled. Lower VG representation was detected in the cortex as compared to the putamen, but relatively more VG copies were seen in mCTX than in other cortical areas. High VG levels were associated with enhanced HTT knockdown in putamen, caudate, cortex, and thalamus. Group A3 showed the highest VG distribution and demonstrated the greatest HTT mRNA knockdown of each of the four brain areas sampled. Lastly, AAV1-VOYHT1 reduced HTT mRNA levels in striatum and primary motor cortex in a volume-dependent manner.

To evaluate vector genome (VG) levels in the cortex, 18 tissue punches were harvested from each animal (9 punches per hemisphere) including 6 punches each from motor, somatosensory, and temporal regions of the cortex (mCTX, ssCTX, and tCTX, respectively). Fifty-four cortex punches in total were evaluated for vector genome (VG) biodistribution per group. The average number of VG copies per cell was calculated for all 18 cortex punches per animal, which were comprised of 6 punches each from mCTX, ssCTX, and tCTX. An average vector genome level of 1.48±0.18 VG copies/cell was observed in the cortex at the highest volume (Group A3), 0.80±0.45 VG copies/cell at the mid volume (Group A2), and 0.84±0.35 VG copies/cell at the low volume (Group A1).VG copies were below the quantification limit (BLQ) for Group A6 (vehicle control). The average number of VG copies per cell detected in combined mCTX, ssCTX, and tCTX punches from each group averaged per animal is presented in Table 56.

TABLE 56
Vector genome copies in combined mCTX, ssCTX, and tCTX
punches across infusion volumes averaged per animal

| | | Sample | VG copies/cell |
|---|---|---|---|
| Group | Description | size | (mean ± stdev) |
| Group A1 | Low Vol; High Conc. | 3 | 0.84 ± 0.35 |
| Group A2 | Mid Vol; High Conc. | 3 | 0.80 ± 0.45 |
| Group A3 | High Vol; High Conc. | 3 | 1.48 ± 0.18 |
| Group A6 | Vehicle Control | 3 | BLQ |

Together, each AAV1-VOYHT1 group (Groups A1, A2 and A3) exhibited a higher number of VG copies/cell as compared to the vehicle group (Group A6; p<0.05), but not from each other (p>0.05). The high-volume group (Group A3) exhibited a numerically greater number of VG copies/cell than the mid- and low-volume groups (Groups A2 and A1, respectively, whereas the mid- and low-volume groups showed similar VG copies/cell to each other.

v. HTT Knockdown and Vector Genome (VG) Measurement in Punches from NHP Striatum and Thalamus at Mid and Low Concentrations

This analysis was designed to evaluate the impact of mid dose concentration, which can also be referred to as medium dose concentration, and low dose concentration on vector distribution and coverage, Selected brain slabs containing the motor and somatosensory cortex and anterior putamen from Groups A4 (mid concentration), A5 (low concentration), and A6 (control) were used to collect 2 nun punches. Six cortex, 8 putamen, 2 caudate, and 5 thalamus punches were collected from each side of the brain (42 total per animal), with a total number of 504 punches collected from all four groups. Samples were homogenized in QuantiGene® homogenization buffer and subjected to protease K digest. Cleared cell lysates were generated and processed for both HTT mRNA measurement using a branched DNA (bDNA) assay and vector genome (VG) measurement using digital droplet PCR (ddPCR) after an additional DNA purification step (Qiagen, catalog#69506). The bDNA assay was carried out according to the QuantiGene® Plex Assay (ThermoFisher Scientific) protocol using a probe set specifically for rhesus HTT. Cell lysate was assayed in duplicate. HTT mRNA level was normalized to the geometric mean of three rhesus housekeeping genes, i.e. AARS, TBP, and XPNPEP1. Results were calibrated to the normalized mean of the vehicle group and presented as: mean of relative remaining HTT mRNA (%)±stdev. For ddPCR, whole cell DNA was prepared from same tissue homogenate used in the bDNA assay. The level of vector genome detected with probe set, CBA Promoter, was normalized to a Host probe set (RNase P). All samples were blinded during the analysis.

In the putamen, both Group A4 (mid concentration) and Group A5 (low concentration) showed HTT mRNA knockdown, with 63±9% (mean±stdev) and 73±9% HTT mRNA remaining relative to control, respectively. Thus, mRNA levels were reduced in a dose-associated manner, with an approximate 37% and 27% reduction in HTT mRNA for mid and low concentration groups, respectively. For VG levels, the average number of VG copies detected in putamen punches for Group A4 and Group A5 were 119.4±18.1 and 66.9±21.5 VG copies/cell, respectively.

HTT mRNA knockdown was also observed in the caudate, with 88±6% (mean±stdev) and 91±10% knockdown relative to control for Groups A4 and A5, respectively. Thus, mRNA levels were reduced in a dose-associated manner, with an approximate 12% and 9% reduction in HTT mRNA for mid and low concentration groups, respectively. HTT mRNA reduction was about 20% lower in the caudate versus the putamen for both Group A4 and Group A5. For VG levels, the average number of VG copies detected in caudate punches from Group A4 and Group A5 was 0.4±0.1 and 9.3±15.4 VG copies/cell, respectively. When a Grubbs' test (Q=0.1%) was applied to remove outliers, the average number of VG copies detected in caudate punches from Group A5 went from 9.3 to 0.3±0.2. Average VG for Group A4 remained unchanged after the Grubbs' test. VG copy representation was several-fold lower in the caudate than in the putamen at both medium (˜300-fold lower) and low (˜7-fold lower) dose concentrations.

Lastly, HTT mRNA knockdown was observed in the thalamus, with 59±20% (mean stdev) and 52±13% knockdown relative to control for Groups A4 and A5, respectively. HTT mRNA levels were reduced in the thalamus by approximately 41% and 48% for mid and low concentration groups, respectively. While an emerging relationship between HTT mRNA knockdown and dose concentration was observed in the striatum, this was not the case in thalamus, where the mid dose concentration was associated with lower mRNA knockdown levels than the low dose concentration. For VG levels, the average number of VG copies detected in thalamus punches from Group A4 and A5 was 416.0±149.3 and 246.7±87 VG copies/cell, respectively. VG representation was higher in the thalamus than in the striatum at both mid and low dose concentrations.

Together, mid and low AAV1-VOYHT1 concentrations were associated with reduced HTT mRNA levels in striatum (putamen and caudate) and thalamus. HTT mRNA knockdown was higher in the thalamus compared to both the putamen and caudate. In the striatum, HTT mRNA knockdown was approximately 20% greater in the putamen versus the caudate. The mid AAV1-VOYHT1dose was associated with greater HTT knockdown than the low AAV1-VOYHT1dose in the striatum, but not in the thalamus, where knockdown was about 45% regardless of dose. Of the three brain regions assessed, the number of VG copies per cell was highest in the thalamus and lowest in the caudate.

vi. HTT Knockdown and Vector Genome (VG) Measurement in Laser Captured (LC) Neurons from NHP Cortex

Selected brain slabs from Group A3 (high vol; high conc.) and Group A6 (vehicle control) were processed to isolate primary motor cortex (mCTX) and somatosensory cortex (ssCTX) samples. Samples were cut into 14 μm sections and stained with 1% cresyl violet. Cortical pyramidal neurons were captured using laser capture microdissection (LCM). For the 1^{st} LCM analysis, one mCTX and one ssCTX sample were collected from each side of the brain (4 samples per animal), with a total of 24 samples collected. Two sets of 750 pyramidal neurons in cortex layers V and VI were laser captured (LC) and homogenized in 50 μl lysis buffer, pooled to a total of 100 μl. For the 2^{nd} LCM analysis, two mCTX and four ssCTX sample were collected from each side of the brain (12 samples per animal), with a total of 72 samples collected. Nine-hundred pyramidal neurons were laser captured from cortical layers V and VI from each sample. Each sample was initially isolated using ARCTURUS® PicoPure® RNA Isolation Kit (Thermo Fisher Scientific, Cat. No. KIT0204) and subsequently processed for both HTT mRNA level using quantitative reverse transcription-PCR (RF-qPCR) and vector genome (VG) level using digital droplet PCR (ddPCR), after an additional DNA purification step (Qiagen, catalog#56304), For RT-gPCR, all samples were analyzed with TaqMan™ PreAmp Master Mix (Thermo Fisher Scientific, Cat. No. 4391128). Calculations across the data sets were carried out according to Vandesompele J et al., Genome Biol. 2002;3(7):RESEARCH0034. HTT mRNA level was normalized to the geometric mean of three rhesus housekeeping genes, i.e. AARS, TBP. and XPNPEPL Results were calculated as fold HTT mRNA relative to the average of all vehicle samples in a given tissue. For ddPCR, the level of vector genome detected with probe set, CBA Promoter, was normalized to a Host probe set (RNase P). All samples were blinded during the analysis.

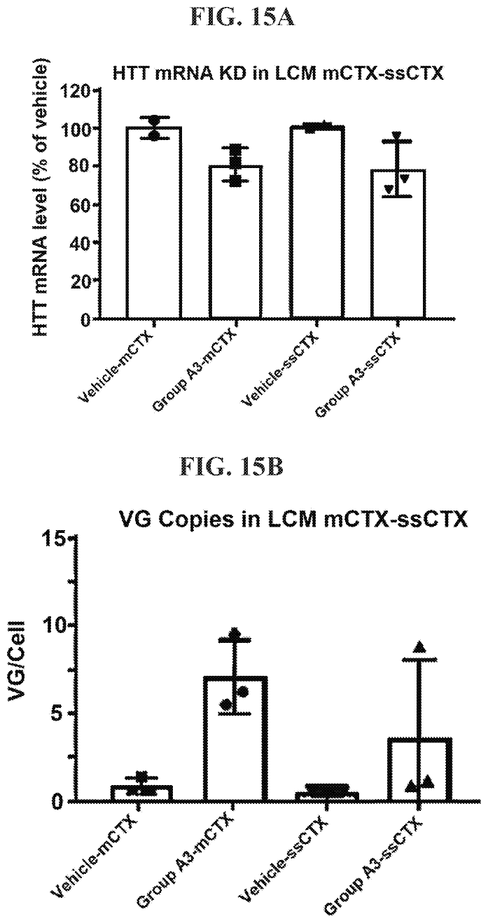

From the 1^{st} LCM analysis, modest HTT mRNA knockdown (19% in mCTX and 23% in ssCTX) was achieved in Group A3 (highest volume and concentration) (see FIG. 15A). Approximately 3-7 VG copies per cell correlated with modest HTT mRNA KD in mCTX and ssCTX pyramidal neurons (see FIG. 15B). Compared to tissue punches from mCTX and ssCTX (see above), more HTT knockdown and vector genome copies were detected in LCM samples.

From the 2^{nd} LCM analysis, HTT mRNA levels and VG levels normalized to vehicle are presented in Tables 57-60, Data shown are mean±stdev for all mCTX or ssCTX samples from a group or accounted for individual animals in a group (3 NHPs per group). Combined mCTX and ssCTX pyramidal neuron samples were also assessed for individual animals in a group, as shown in Tables 57 and 59. Modest, but significant HTT mRNA knockdown (21% in mCTX and 23% in ssCTX) was achieved in Group A3. Average 2.79 and 1.36 VG copies per cell were detected in LC pyramidal neurons from mCTX and ssCIX, respectively. Better HTT mRNA knockdown was seen in LCM samples compared to the tissue punches (14% in mCTX and 6% in ssCTX). The readouts of HTT mRNA knockdown from 2nd LCM analysis was consistent with 1st LCM results, while VG copy number measured in the 2nd LCM was slightly lower than that in the 1st LCM analysis.

TABLE 57
HTT mRNA level in all LC neurons
of mCTX and ssCTX (2^{nd} LCM)

| | | Relative HTT mRNA level (% of | |
|---|---|---|---|
| | | vehicle) (mean ± stdev) | |
| Group | Description | mCTX (n = 12) | ssCTX (n = 24) |
| Group A3 | high vol; high conc. | 76 ± 11 | 77 ± 14 |
| Group A6 | vehicle control | 100 ± 37 | 100 ± 18 |

TABLE 58
HTT mRNA level in LC neurons of mCTX
and ssCTX of each animal (2^{nd} LCM)

| | | Relative HTT mRNA level (% of | | |
|---|---|---|---|---|
| | | vehicle) (mean ± stdev, N = 3) | | |
| | | | | mCTX + |
| Group | Description | mCTX | ssCTX | ssCTX |
| Group A3 | high vol; high conc. | 79 ± 11 | 77 ± 8 | 77 ± 13 |
| Group A6 | vehicle control | 100 ± 37 | 100 ± 17 | 100 ± 25 |

TABLE 59
VG level in all LC neurons of mCTX and ssCTX (2^{nd} LCM)

| | | VG copies/cell (mean ± stdev) | |
|---|---|---|---|
| Group | Description | mCTX (n = 12) | ssCTX (n = 24) |
| Group A3 | high vol; high conc. | 2.79 ± 1.55 | 1.36 ± 0.97 |
| Group A6 | vehicle control | 0.18 ± 0.11 | 0.46 ± 0.3 |

TABLE 60
VG level in LC neurons of mCTX and
ssCTX of each animal (2^{nd} LCM)

| | | VG copies/cell (mean ± | | |
|---|---|---|---|---|
| | | stdev, N = 3) | | |
| | | | | mCTX + |
| Group | Description | mCTX | ssCTX | ssCTX |
| Group A3 | high vol; high | 2.79 ± 0.08 | 1.36 ± 0.28 | 1.84 ± 0.17 |
| | conc. | | | |
| Group A6 | vehicle control | 0.17 ± 0.05 | 0.45 ± 0.17 | 0.37 ± 0.12 |

vii. In Situ Hybridization (ISH) for VG and HTT mRNA in NHP Motor and Somatosensory

Selected brain slices containing the motor and somatosensory cortex from Group A3 (high vol; high cone.) and Group A6 (vehicle control) animals were processed for in situ hybridization (ISH) using the BaseScope™ Assay to detect vector genome DNA and HTT mRNA. Five pm-thick formalin-fixed paraffin-embedded (FFPE) brain sections were incubated with BaseScope™ ISH target-specific probes for Macaca mRNA (GenBank Accession Number: XM_015137840.1) and the AAV1 vector genome. Three pairs of double Z probes were used for HTT mRNA, and these probes were designed against 3 exon junctions in the HTT gene. Four pairs were used for the vector genome, and these probes were designed against multiple non-pri-miRNA regions. Positive control probes, BA-Mmu-PPIB-3zz (Peptidylprolyl Isomerase B (Cyclophilin B), Cat. No. 708161), and a negative control probe, BA-dapB-3zz (Cat. No. 701011), were also added. Signal amplification and tissue staining were carried out using BaseScope™ Red Reagent Kit (Cat. No. 322910). Images were detected and analyzed under a microscope for vector genome and HTT mRNA levels.

Quantification of BaseScope™ ISH results was performed with Image) imaging analysis software. The scoring criteria used for evaluation of BaseScope™ staining is listed in Table 61, Scoring was performed at 40×magnification. Scoring was performed based on the number of dots per cell rather than the signal intensity, since dots correlate to the number of individual target molecules, whereas dot intensity reflects the number probe pairs bound to each molecule. AAV vector biodistribution was calculated as the percentage of cells with dots relative to the total number of cells in a specific cortex region. For vector genome readouts, only nuclear signals were counted.

TABLE 61
Scoring criteria for ISH staining

| Score | Criteria |
|---|---|
| 0 | No staining or <1 dot/10 cells |
| 1 | 1-3 dots/cell |
| 2 | 4-9 dots/cell, no or very few dot clusters |
| 3 | 10-15 dots/cell and <10% dots are in clusters |
| 4 | >15 dots/cell and >10% dots are in clusters |

For vector biodistribution, extensive vector genomes were detected at the injection sites (thalamus and putamen) in Group A3. In the cortex, an average of 18% mCTX and 9% ssCTX cells with detectable AAV vector in the nucleus was achieved in Group A3. More cells with detectable vector genome were observed in the mCTX than in the ssCTX, with a combined average of 12.48% vg+ cells in both the mCTX and ssCTX in NHPs. The results of vector biodistribution in NHP cortex based on vector genome ISH are presented in Tables 62 and 63.

TABLE 62
VG distribution per cortical region in mCTX and ssCTX

| | | % of cells with VG (mean ± stdev) | |
|---|---|---|---|
| Group | Description | mCTX (n = 18) | ssCTX (n = 30) |
| Group A3 | high vol; high conc. | 17.87 ± 7.04 | 9.35 ± 2.80 |
| Group A6 | vehicle control | 0.75 ± 1.51 | 0.72 ± 0.92 |

TABLE 63
VG distribution per animal in mCTX and ssCTX

| | | % of cells with VG (mean ± | |
|---|---|---|---|
| | | stdev, N = 3) | |
| Group | Description | mCTX | ssCTX |
| Group A3 | high vol; high conc. | 17.87 ± 2.94 | 9.36 ± 0.29 |
| Group A6 | vehicle control | 0.75 ± 1.12 | 0.72 ± 0.53 |

For VG levels, average vector genome scorings were ˜1 for cells in both mCTX and ssCTX in NHPs of Group A3 (high volume, high concentration) dosed with AAV1-VOYHT1 by bilateral thalamus and putamen infusion. The results of VG scoring in NHP cortex using the scoring criteria set forth above are presented in Tables 64 and 65.

TABLE 64
VG score per cortical region in mCTX and ssCTX

| | | VG score (mean ± stdev) | |
|---|---|---|---|
| Group | Description | mCTX (n = 18) | ssCTX (n = 31) |
| Group A3 | high vol; high conc. | 1.01 ± 0.02 | 1.08 ± 0.13 |
| Group A6 | vehicle control | 0 | 0 |

TABLE 65
VG score per animal in mCTX and ssCTX

| | | VG score (mean ± stdev, N = 3) | |
|---|---|---|---|
| Group | Description | mCTX | ssCTX |
| Group A3 | high vol; high conc. | 1.01 ± 0.01 | 1.08 ± 0.07 |
| Group A6 | vehicle control | 0 | 0 |

For HTT mRNA levels, HTT mRNA scores in both mCTX and ssCTX of the AAV1-VOYHT1-treated group showed significantly lower scores than that in the vehicle group, indicating a significant reduction of HTT mRNA level caused by AAV1-VOYHT1 treatment. The results of HTT mRNA scoring in NHP cortex using the scoring criteria set forth above are presented in Tables 66 and 67.

TABLE 66
HTT mRNA scores per cortical region in mCTX and ssCTX

| | | HTT mRNA scores (mean ± stdev) | |
|---|---|---|---|
| Group | Description | mCTX (n = 18) | ssCTX (n = 30) |
| Group A3 | high vol; high conc. | 1.51 ± 0.22 | 1.91 ± 0.28 |
| Group A6 | vehicle control | 2.24 ± 0.38 | 2.26 ± 0.28 |

TABLE 67
HTT mRNA scores per animal in mCTX and ssCTX

HTT mRNA score (mean ±
stdev, N = 3)

| Group | Description | mCTX | ssCTX |
|---|---|---|---|
| Group A3 | high vol; high conc. | 1.51 ± 0.15 | 1.91 ± 0.25 |
| Group A6 | vehicle control | 2.24 ± 0.28 | 2.26 ± 0.2 |

viii. Clinical Signs and Histopathology

Minimal to mild clinical signs were observed in 7 out of 18 test animals, including mild incoordination, inappetence, decreased feed, and overall weakness. Histopathology analysis overall showed safety at the tested doses. Low levels of mononuclear cell infiltrates were detected in the putamen and thalamus. The degree of infiltration of mononuclear cells corresponded proportionately to the infusion volume. Necrosis was most pronounced in the vehicle group. Minimal damage was observed in parietal cortex and occipital cortex.

ix. Summary

These data suggested that attaining the targeted levels of HTT knockdown in cortex via intrathalamic and/or intraputaminal infusion is achievable upon using optimal dosing paradigm. AAV1-VOYHT1 was well-tolerated based on clinical signs and histological assessment of the brain.

### Example 2

### Dose Optimization Study II

This study was carried out to further evaluate delivery parameters to optimize coverage of AAV1-VOYHT1 in NHP brain, and to extrapolate parameters to clinical dosing paradigm. This study utilized a total of 10 animals, which were assigned to 4 treatment groups as summarized in Table 68. Animals received bilateral parenchymal infusion (4 infusions) into the putamen and thalamus of AAV1-VOYHT1. with increased dosing compared to Example 1, Experimental procedures were similar to that described in Example 1. Animals were euthanized 5 weeks after dosing, and tissues were collected for post-mortem analysis.

TABLE 68
Study design

| | | Number of | Volume (μL/side) | | Dosing Titer | Total |
|---|---|---|---|---|---|---|
| Group | Description | animals | Putamen | Thalamus | (vg/ml) | dose (vg) |
| Group B1 | High vol. vehicle | 2 | 350 | 500 | 0 | 0 |
| Group B2 | Mid vol. Mid Conc. | 3 | 250 | 325 | 4e12 | 4.6e12 |
| Group B3 | Mid vol. High Conc. | 3 | 250 | 325 | 7.9e12 | 9.1e12 |
| Group B4 | Mid vol. Vehicle | 2 | 250 | 325 | 0 | 0 |

The calculated human equivalent dose corresponding to each dosing group from Table 67 is presented in Table 69.

TABLE 69
Human equivalent dose

| | | Human Equivalent Dose | | |
|---|---|---|---|---|
| | | Putamen | Thalamus | |
| Group | Description | (μL/side) | (μL/side) | dose (vg) |
| Group B1 | High vol. vehicle | 1225 | 3000 | 0 |
| Group B2 | Mid vol. Mid Conc. | 875 | 1950 | 2.3e13 |
| Group B3 | Mid vol. High Conc. | 875 | 1950 | 4.5e13 |
| Group B4 | Mid vol. Vehicle | 875 | 1950 | 0 |

Side effects were observed post-dosing, which were likely due to intolerance to large infusion volumes. Disuse of one or both hindlimbs was observed in the animals dosed with the high-volume vehicle control (Group B1), In two animals that received medium volume AAV1-VOYHT1 treatment (Groups B2, B3), clinical signs such as paresis in both legs, prone/ambulating slowing, head tilt were observed. MRI observations showed some reflux along both cannula tracts in three animals.

Histopathology analysis showed slight gliosis and necrosis in the putamen (unavoidable due to placement of catheter) in the vehicle group. In Group B2 animals, a notable increase was seen in mononuclear cell infiltrates at both putamen and thalamic infusion sites, but these were not expected to result in clinical signs. Slight increases in gliosis and necrosis in both structures were observed but neither finding was expected to result in any clinical signs. Edema was also observed. In Group B3 animals, slight increases in gliosis and necrosis were seen in both structures relative to control but were considered of no biologic relevance. Mononuclear infiltrates were increased compared to the vehicle group. An increase in edema was also observed, but this was not expected to cause any clinical signs.

### Example 3

### Dose Optimization Study III

i. Study Design

The primary goals of this study were to demonstrate HTT mRNA knockdown in NHP cortex with AAV1-VOYHT1 and to demonstrate safety for thalamic-only and combined thalamic and putaminal infusion paradigms. The secondary goals were to show a correlation between VG and HTT mRNA levels in laser captured (LC) pyramidal neurons from primary motor and somatosensory cortex; demonstrate a correlation between HTT mRNA and VG levels in tissue punches from the putamen, thalamus, and caudate; demonstrate a correlation between HTT protein and HTT mRNA levels in putamen; measure AAV1-VOYHT1 specific miRNA expression levels in tissue punches from putamen and caudate; demonstrate a correlation between vector genome (VG) and AAV1-VOYHT1 specific miRNA expression levels in tissue punches from putamen and caudate; and demonstrate a correlation between HTT mRNA and AAV1-VOYHT1 specific miRNA expression levels in tissue punches from putamen and caudate.

This study was implemented in two phases. A total of 15 male and female rhesus macaques were assigned to 5 groups with 3 animals per group (see Table 57). In Phase I, the first group of animals (Group C1a) were dosed with a vehicle control by intraparenchymal injection bilaterally into both the thalamus and putamen using MRI-guided convection enhanced delivery (CED) to establish infusion parameters (e.g., rate, volume and duration) before proceeding to Phase II of the study. A second group (Group C1b) was dosed with refined surgical procedures and served as the control group for the treatment groups. After the infusion parameters were established in Phase I, they were used for dosing the test article containing AAV1-VOYHT1 in the three treatment groups. The first treatment group (Group C2) received bilateral infusion of the test article into the thalamus only using MRI-guided CED. This group was dosed to demonstrate the safety and Huntingtin (HTT) mRNA knockdown (KD) in cortical pyramidal neurons in the primary motor and somatosensory cortex by laser capture microdissection (LCM) after thalamus infusion only. Next, in the other two treatment groups (Group C3 and Group C4), the test article was infused bilaterally into both the thalamus and putamen at 2 different dose levels for dose optimization.

The study schedule was as follows. In Phase I, the first vehicle group (Group C1a) was dosed using pre-selected infusion parameters, A Functional Observation Battery (FOB) evaluation focusing on neurological status was carried out 5±2 days post-infusion and 3±days prior to termination. An additional 3 animals (Group C1b) were dosed and then evaluated with FOB at 5±2 days after dosing and 3±days prior to termination, as per Group C1a. In Phase II, all animals (N=9) were dosed with the test article containing AAV1-VOYHT1 in accordance with the infusion parameters established in Phase I. Group C2 (thalamus only) was dosed first, followed by Group C3 at a medium dose and then Group C4 at a high dose. Except for Group C2 in which the animals received bilateral thalamic dosing only, each animal received bilateral intracranial infusion of vehicle or test article into the putamen and thalamus. An intraparenchymal dosing paradigm was employed in which 2-4 infusions (1infusion per structure) were given at a speed of up to 5 μL/min, A baseline neurological FOB evaluation was performed on each animal prior to dosing followed by a second FOB evaluation of each animal at 5±2 days after dosing. When the second FOB was satisfactory, the animal was euthanized at Dav 36±3 (˜5 weeks in-life duration) and a third FOB evaluation was performed 3±2 days prior to necropsy. Tissues were collected for post-mortem analysis.

A summary of the study design is shown in Table 70. For the high dose group (Group C4), the total dose of 1.8e13 vg was calculated based on the maximal titer (2.2e13 vg/ml) achieved.

TABLE 70
Study design

| | | | | | | | Total |
|---|---|---|---|---|---|---|---|
| | | Number of | | Volume (μL/side) | | Dosing Titer | dose |
| Phase | Group | animals | Description | Putamen | Thalamus | (vg/ml) | (vg) |
| I | Group C1 (a/b) | 3 + 3 | Vehicle | 150 | 250 | 0.0 | 0.0 |
| II | Group C2 | 3 | Thalamus only | — | 250 | 2.2e13 | 1.1e13 |
| | Group C3 | 3 | Medium dose | 150 | 250 | 1.0e13 | 8.0e12 |
| | Group C4 | 3 | High dose | 150 | 250 | 2.2e13 | 1.8e13 |

ii. Animal Care and Sample Collection

Eighteen (N=18) adult male or female rhesus macaques (4-11 years old) were selected based on anti-AAV1 neutralizing antibody (nAb) serum titers 15 days prior to the start of ambulation training. Selected candidates for Groups C2, C3 and C4 exhibited low AAV1 nAb in general. Animals weighed 5-14 kg. Ambulation training was carried out daily for up to 4 consecutive weeks before animal enrollment. Animals were weighed and assigned by nAb status to the study groups as summarized in Table 57, The 3 animals selected as backups were kept as spares until the completion of dosing. Animal husbandry conditions were similar as described in Example 1.

Blood samples were collected for clinical pathology evaluation and neutralizing antibody (nAb) analysis at Day 1 (Pre-dose), Day 15±2, and immediately prior to necropsy on Day 36±3. The clinical pathology evaluation included hematology (CBC), serum clinical chemistry (Chem), and coagulation (Coag) analysis. Cerebrospinal fluid (CSF) samples were collected for nAb analysis from the cervical region at Day 1 (Pre-dose), Day 15±2, and immediately prior to necropsy on Day 36±3. Following necropsy, the brain and selected peripheral organs were collected and then fresh frozen or 4% paraformaldehyde (PFA) post-fixed by immersion.

iii. Test Article Preparation and Dosing Procedures

The test article used in the study contained AAV1-VOYHT1 gene transfer vector formulated in Phosphate Buffered Saline with 5% sucrose and 0.001% Poloxamer 188 (Pluronic® F-68). The vehicle control contained the formulation buffer only. The samples were stored at -60 CC or below and were thawed to and maintained at 2-8° C. on day of dosing. ProHance® (Bracco Diagnostics, Inc), i.e. gadoteridol, was added at a 1:250 ratio (1 μL of ProHance per 250 μL of test article or control) and carefully mixed by inverting tubes prior to loading into the infusion system. The dosing solution contained the test article or control and a 2 mM concentration of gadoteridol.

Immediately prior to surgery, each animal was anesthetized with intramuscular (IM) ketamine (10 mg/kg) and IM dexmedetomidine (15 μg/kg), weighed, hair of head and neck shaved, intubated, and maintained on 1-5% isoflurane. The animal's head was secured onto a stereotaxic frame containing one MRI surface coil on each side of the ear bars and then transferred to the MRI to acquire a baseline scan. A T1- and T2-weighted MRI sequences were acquired and used to determine coordinates of the central sulcus. Next the animal was transferred back to the surgery suite and the head prepared for the neurosurgical implantation procedure. Using aseptic techniques, the wound site was opened in anatomical layers to expose the skull. Depending on which dose group, craniotomies were performed at entry sites located above the parietal and/or occipital lobe on each side. A skull mounted cannula guide ball array was temporarily secured to the skull over each burr hole using titanium screws. Immediately after implantation of ball arrays, the animal was transferred to the MRI suite. MR imaging was used to align cannula guides with putamen and/or thalamus targets ipsilateral to each cannula guide. Test article or control was administered with repeated MR imaging to visually monitor infusions within the brain as specified in Table 57 (above). Each animal received infusions (sites) of the test article or control using convection enhanced delivery (CED) in each putamen (except Group C2) and thalamus. A 16G cannula (Mill Interventions Inc.) was primed with dosing solution and guided into each target site through the skull mounted ball arrays. Each cannula was connected via microbore extension lines (Smiths Medical) to a 3-6cc syringe mounted on a syringe pump (Harvard Apparatus). Dose rates, durations and volumes administered into each putamen and thalamus using ascending infusion rates in 3 different stages of intraparenchymal infusion are listed in Table 71. “N/A” indicates data not applicable.

TABLE 71
Infusion parameters

| | Putamen | | | Thalamus | | |
|---|---|---|---|---|---|---|
| Stage of | Rate | Duration | Volume | Rate | Duration | Volume |
| infusion | (μl/min) | (min) | (μl) | (μl/min) | (min) | (μl) |
| 1 | 1 | 16 | 16 | 1 | 25 | 25 |
| 2 | 3 | 28 | 84 | 3 | 50 | 150 |
| 3 | 5 | 20 | 50 | 5 | 15 | 75 |
| Total | N/A | 64 | 150 | N/A | 90 | 250 |

Serial MRI scans were acquired to monitor infusate distribution within each target site and provide real-time monitoring of the dosing. In some cases, cannula was advanced deeper into the putamen or thalamus during the infusion to maximize infusate distribution within the putamen or thalamus. Distribution of infusate into CNS structures adjacent to the putamen and thalamus was anticipated and intended to occur due to the total volume to be delivered per site. Immediately after the MRI CED dosing procedure, the animal was transferred back to the surgery suite, the ball array system was explanted and the wound site closed in anatomical layers with absorbable vicryl suture using a simple interrupted suturing pattern. Pre- and post-operative medications included atipamezole (0.03 mL/kg, IM), buprenorphine (0.03 mg/kg, IM, b.i.d.), carprofen (2.2 mg/kg SQ, b.i.d.), ketoprofen (2 mg/kg, IM, s.i.d.), and cefazolin (100 mg IV, pre- and post-surgery, followed by 25 mg/kg, IM, b.i.d.) or ceftriaxone (50 mg/kg, IM, Animals were monitored for full recovery from anesthesia and returned to their home cages.

iv. HTT Knockdown and VG Measurement in LC Neurons from Combined NHP mCTX and ssCTX

Selected brain slabs from three groups (Group C1, Group C3 and Group C4) were processed to isolate primary motor cortex (mCTX) and somatosensory cortex (ssCTX) samples by laser capture microdissection (LCM). A total of 54 mCTX samples and 90 ssCTX LCM samples were collected. Each LCM sample contained 900 pyramidal neurons laser captured (LC) from cortical layers V and VI, with a total of 129,600 neurons captured. Samples were processed for both HTT mRNA level using RT-qPCR and vector genome (VG) level using ddPCR, as described in Example 1. All samples were blinded during the analysis.

For HTT mRNA knockdown, the relative HTT mRNA levels in LC neurons from combined mCTX and ssCTX of AAV1-VOYHT1-treated groups after normalization to the vehicle control group are presented in Table 71, The greatest HTT knockdown in combined samples of LC pyramidal neurons from mCTX and ssCTX (32%) was observed in Group C4 (high dose bilateral putamen+thalamus group), with less HTT knockdown (13%) in Group C3 (medium dose bilateral putamen thalamus group). An average of 30% HTT mRNA knockdown was observed in mCTX and 33% HTT mRNA knockdown in ssCTX in Group C4. HTT knockdown was approximately dose-proportional (2.25×greater dose resulted in 2.9×greater knockdown). The percentage of samples of LC cortical neurons that exhibited over 30% HTT knockdown is also shown in Table 72. In LC motor and somatosensory cortical neurons, 58% of samples showed ≥30% HTT knockdown and 27% of samples showed ≥40% HTT knockdown in Group C4 (high dose putamen+thalamus group), whereas 36% of samples showed ≥30% HTT knockdown and 7% of samples showed ≥40% HTT knockdown in Group C3 (medium dose putamen+thalamus group). Thus, HTT mRNA knockdown in motor and somatosensory cortical neurons is dependent on the concentration of AAV1-VOYHT1 infused into thalamus and putamen. In addition, over 40% of LCM mCTX samples showed ≥30% HTT mRNA knockdown in both medium and high dose groups, while 60% of LCM ssCTX samples in the high dose group showed ≥30% HTT mRNA knockdown.

TABLE 72
HTT knockdown in LC neurons from combined mCTX and ssCTX

| | | HTT mRNA | | |
|---|---|---|---|---|
| | | relative to | | |
| | | vehicle (% + | % LCMs ≥ | % LCMs ≥ |
| Group | Description | stdev, N = 3) | 30% KD | 40% KD |
| Group C1 | Vehicle | 100 ± 10 | 5 | 0 |
| Group C3 | Medium dose | 87 ± 26 | 36 | 7 |
| Group C4 | High dose | 68 ± 3 | 58 | 27 |

For VG levels, LC neuron samples from combined mCTX and ssCTX showed a dose-dependent increase in VG copies per cell, as shown in Table 73. The number of VG copies per cell was approximately 30 copies/cell in the high dose group. VG copies tracked with HTT mRNA knockdown such that a higher number of VG copies corresponded to greater HTT mRNA knockdown.

TABLE 73
VG levels in LC neurons from combined mCTX and ssCTX

| | | VG copies/cell |
|---|---|---|
| Group | Description | (mean ± stdev, N = 3) |
| Group C1 | Vehicle | 0.54 ± 0.55 |
| Group C3 | Medium dose | 8.00 ± 0.71 |
| Group C4 | High dose | 28.55 ± 23.16 |

v. HTT Knockdown and VG Measurement in LC Neurons from AHP mCTA7

Selected brain slabs from three groups (Group C1, Group C3 and Group C4) were processed to isolate primary motor cortex (mCTX) samples by laser capture microdissection (LCM), A total of 54 mCTX samples were collected. Each LCM sample contained 900 pyramidal neurons laser captured (LC) from cortical layers V and VI. Samples were processed for HTT mRNA level using RT-qPCR and vector genome (VG) level using ddPCR, as described in Example 1. All samples were blinded during the analysis.

For HTT mRNA knockdown, the relative HTT mRNA levels in LC neurons from mCTX of AAV1-VOYHT1-treated groups after normalization to vehicle control group are presented in Table 74. The greatest HTT knockdown in LC pyramidal neurons from mCTX (30%) was observed in Group C4 (high dose bilateral putamen+thalamus group), with less HTT knockdown (13%) in Group C3 (medium dose bilateral putamen+thalamus group).

TABLE 74
HTT knockdown in LC neurons from mCTX

| | | HTT mRNA |
|---|---|---|
| | | relative to vehicle |
| Group | Description | (mean ± stdev, N = 3) |
| Group C1 | Vehicle | 100 ± 11 |
| Group C3 | Medium dose | 87 ± 27 |
| Group C4 | High dose | 70 ± 7 |

For VG levels, LC neurons from mCTX showed a dose-associated increase in VG copies per cell, as shown in Table 75, The number of VG copies per cell reached approximately 20 copies/cell in Group C4 (high dose bilateral putamen+thalamus group), and 10 copies/cell in Group C3 (medium dose bilateral putamen-F-thalamus group). Thus, VG copies tracked with HTT mRNA knockdown such that a higher number of VG copies corresponded to greater HTT mRNA knockdown

TABLE 75
VG levels in LC neurons from mCTX

| | | VG copies/cell |
|---|---|---|
| Group | Description | (mean ± stdev, N = 3) |
| Group C1 | Vehicle | 0.15 ± 0.03 |
| Group C3 | Medium dose | 9.61 ± 1.59 |
| Group C4 | High dose | 21.6 ± 0.74 |

vi. HTT Knockdown and VG Measurement in LC Neurons from AUIP ssCTX

Selected brain slabs from three groups (Group C1, Group C3 and Group C4) were processed to isolate somatosensory cortex (ssCTX) samples by laser capture microdissection (LCM), A total of 90 ssCTX LCM samples were collected. Each LCM sample contained 900 pyramidal neurons laser captured (LC) from cortical layers V and VI. Samples were processed for HTT mRNA level using RI-qPCR and vector genome (VG) level using ddPCR, as described in Example 1. All samples were blinded during the analysis.

For HTT mRNA knockdown, the relative HTT mRNA levels in LC neurons from ssCTX of AAV1-VOYHT1-treated groups after normalization to vehicle control group are presented in Table 76. The greatest HTT knockdown in LC pyramidal neurons from ssCTX (33%) was observed in Group C4 (high dose bilateral putamen+thalamus group), with less HTT knockdown (13%) in Group C3 (medium dose bilateral putamen+thalamus group).

TABLE 76
HTT knockdown in LC neurons from ssCTX

| | | HTT mRNA |
|---|---|---|
| | | relative to vehicle |
| Group | Description | (mean ± stdev, N = 3) |
| Group C1 | Vehicle | 100 ± 8 |
| Group C3 | Medium dose | 86 ± 24 |
| Group C4 | High dose | 67 ± 11 |

For VG levels, LC neurons from ssCTX showed a dose-associated increase in VG copies per cell, as shown in Table 77. The number of VG copies per cell reached approximately 33 copies/cell in the Group C4 (high dose bilateral putamen-thalamus group), and 7 copies/cell in Group C3 (medium dose bilateral putamen+thalamus group). Thus, VG copies tracked with HTT mRNA knockdown such that a higher number of VG copies corresponded to greater HTT mRNA knockdown,

TABLE 77
VG levels in LC neurons from ssCTX

| | | VG copies/cell |
|---|---|---|
| Group | Description | (mean ± stdev, N = 3) |
| Group C1 | Vehicle | 0.77 ± 0.89 |
| Group C3 | Medium dose | 7.01 ± 2.98 |
| Group C4 | High dose | 32.72 ± 37.37 |

The LCM results demonstrated that combined bilateral putaminal and thalamic infusion of AAV1-VOYHT1 resulted in VG delivery and HTT mRNA knockdown in motor and somatosensory cortical pyramidal neurons in medium and high dose groups, with greater vector genome delivery and greater HTT mRNA knockdown in the high dose group.

vii. HTT Knockdown and VG Measurement in Punches from Combined NHP mCTX and ssCTX

Two selected brain slabs containing the motor and somatosensory cortex from all four groups were used to collect 2 mm primary motor cortex (mCTX) and somatosensory cortex (ssCTX) punches. Six mCTX and 6 ssCTX punches were collected per animal, with a total number of 144 punches collected. Equal numbers of punches were collected from each side of the cortex. Samples were processed and analyzed for HTT mRNA and vector genome (VG) using bDNA and ddPCR, respectively, bDNA and ddPCR were carried out as described in Example 1. All samples were blinded during the analysis.

For HTT mRNA knockdown, the relative HTT mRNA levels in LC neurons from combined mCTX and ssCTX in the AAV1-VOYHT1-treated groups after normalization to the vehicle control group are presented in Table 77. An average of 16% HTT knockdown was observed in cortical punches from Group C4 (high dose bilateral putamen+thalamus group). HTT knockdown was dose-proportional (2.25×greater dose resulted in 2.28×greater knockdown) based on Groups C3 and C4. The percentage of punches that exhibited over 20% HTT knockdown in each group is also shown in Table 78. 39% of combined mCTX and ssCTX punches showed ≥20% HTT knockdown, but 72% of mCTX punches showed ≥20% HTT knockdown. Greater HTT knockdown was observed in the high dose putamen+thalamus Group C4 than in the thalamus only Group C2, suggesting that putamen infusion of AAV1-VOYHT1 contributes to HTT knockdown in motor and somatosensory cortex. With thalamus only infusion of AAV1-VOYHT1 (Group C2), there was no significant HTT knockdown in motor and somatosensory cortex.

TABLE 78
HTT knockdown in combined mCTX and ssCTX punches

| | | HTT mRNA relative to | % Punches ≥ |
|---|---|---|---|
| Group | Description | vehicle (% ± stdev, N = 3) | 20% KD |
| Group C1 | Vehicle | 100 ± 8 | 0 |
| Group C2 | Thalamus Only | 93 ± 3 | 14 |
| Group C3 | Medium dose | 93 ± 13 | 11 |
| Group C4 | High dose | 84 ± 5 | 39 |

For VG levels, results are summarized in Table 79. VG levels were dose-dependent and dose-proportional (2.25×greater dose resulted in 3-fold higher vector genome level) for putamen+thalamus groups C3 and C4. Higher VG copies were detected in mCTX than in ssCTX in each group. Higher VG copies were detected in the Group C4 group (high dose putamen thalamus) than in the Group C2 group (thalamus only), suggesting that putamen infusion of AAV1-VOYHT1 contributes to VG copies in motor and somatosensory cortex. VG copies correlated with HTT mRNA knockdown in the punch analysis.

TABLE 79
VG levels in combined mCTX and ssCTX punches

| | | VG copies/cell |
|---|---|---|
| Group | Description | (mean ± stdev, N = 3) |
| Group C1 | Vehicle | 0.02 ± 0.04 |
| Group C2 | Thalamus only | 10.15 ± 4.14 |
| Group C3 | Medium dose | 10.98 ± 6.69 |
| Group C4 | High dose | 22.67 ± 10.69 |

viii. HTT Knockdown and VG Measurement in Punches from NHP mCTX

Two selected brain slabs containing the motor cortex from all four groups were used. to collect 2 mm primary motor cortex (mCTX) punches. Six mCTX punches were collected per animal, with a total number of 72 punches collected. Equal numbers of punches were collected from each side of the cortex. Samples were processed and analyzed for HTT mRNA and vector genome (VG) using bDNA and ddPCR, respectively. bDNA and ddPCR were carried out as described in Example 1. All samples were blinded during the analysis.

For HTT mRNA knockdown, the relative HTT mRNA levels in mCTX punches of AAV1-VOYHT1-treated groups after normalization to vehicle control group are presented in Table 80, The greatest HTT knockdown (28%) was observed in Group C4 (high dose bilateral putamen+thalamus group), with less HTT knockdown (9%) in Group C3 (medium dose bilateral putamen+thalamus group) and Group C2 (10%; thalamus only). While an approximate one-third reduction in HTT mRNA was observed with high dose infusion into bilateral putamen and thalamus, only about a 10% reduction was seen with medium dose infusion into bilateral putamen, as well as with thalamus only infusion.

TABLE 80
HTT knockdown in mCTX punches

| | | HTT mRNA |
|---|---|---|
| | | relative to vehicle |
| Group | Description | (mean ± stdev, N = 3) |
| Group C1 | Vehicle | 100 ± 11 |
| Group C2 | Thalamus only | 90 ± 2 |
| Group C3 | Medium dose | 91 ± 11 |
| Group C4 | High dose | 78 ± 7 |

For VG levels, mCTX punches showed a dose-associated increase in VG copies/cell, as shown in Table 81. The number of VG copies per cell was approximately 32 copies/cell in Group C4 (high dose bilateral putamen-F-thalamus group), and 14 copies/cell in Group C3 (medium dose bilateral putamen+thalamus group). Similar to Group C3, approximately 13 vg copies/cell were seen in Group C2 (thalamus only). In general, VG levels tracked with HTT mRNA knockdown in mCTX punches such that a higher number of VG copies corresponded to greater HTT mRNA knockdown.

TABLE 81
VG levels in mCTX punches

| | | VG copies/cell |
|---|---|---|
| Group | Description | (mean ± stdev, N = 3) |
| Group C1 | Vehicle | 0.0 ± 0.00 |
| Group C2 | Thalamus only | 12.5 ± 4.8 |
| Group C3 | Medium dose | 13.8 ± 7.1 |
| Group C4 | High dose | 31.8 ± 5.7 |

ix. HTT Knockdown and VG Measurement in Punches from NHP ssCTX

Two selected brain slabs containing the somatosensory cortex from all four groups were used to collect 2 mm somatosensory cortex (ssCTX) punches. Six ssCTX punches were collected per animal, with a total number of 72 punches collected. Equal numbers of punches were collected from each side of the cortex. Samples were processed and analyzed for both HTT mRNA and vector genome (VG) using bDNA and ddPCR, respectively. tDNA and ddPCR were carried out as described in Example 1. All samples were blinded during the analysis.

For mRNA knockdown, the relative HTT mRNA levels in ssCTX punches of AAV1-VOYHT1-treated groups after normalization to vehicle control group are presented in Table 82. The greatest HTT knockdown (9%) was observed in Group C4 (high dose bilateral putamen+thalamus group), with less HTT knockdown (5%) in Group C3 (medium dose bilateral putamen+thalamus group) and Group C2 (4%; thalamus only). HTT mRNA knockdown in Group C4 was approximately double that observed in Groups C3 and C2.

TABLE 82
HTT knockdown in ssCTX punches

| | | HTT mRNA |
|---|---|---|
| | | relative to vehicle |
| Group | Description | (mean ± stdev, N = 3) |
| Group C1 | Vehicle | 100 ± 7 |
| Group C2 | Thalamus only | 96 ± 6 |
| Group C3 | Medium dose | 95 ± 16 |
| Group C4 | High dose | 91 ± 6 |

For VG levels, mCTX punches showed a dose-associated increase in VG copies per cell, as shown in Table 83. The number of VG copies/cell was approximately 14 copies/cell in Group C4 (high dose bilateral putamen+thalamus group), and 8 copies/cell in Group C3 (medium dose bilateral putamen+thalamus group). Like Group C3, approximately 8 VG copies/cell were seen in Group C2 (thalamus only). VG levels generally tracked with HTT mRNA knockdown in mCTX punches such that a higher number of VG copies corresponded to greater mRNA knockdown.

TABLE 83
VG levels in ssCTX punches

| | | VG copies/cell |
|---|---|---|
| Group | Description | (mean ± stdev, N = 3) |
| Group C1 | Vehicle | 0.0 ± 0.15 |
| Group C2 | Thalamus only | 7.8 ± 1.8 |
| Group C3 | Medium dose | 8.1 ± 6.1 |
| Group C4 | High dose | 13.5 ± 1.5 |

x. In Situ Hybridization (ISH) for VG and HTT mRNA in NHP Motor and Somatosensory Cortex

Selected brain slabs containing the motor and somatosensory cortex from Group C1 (vehicle group) and Group C4 (high dose—putamen+thalamus group) animals were processed for in situ hybridization (ISH) using the BaseScope™ Assay to detect vector genome DNA and HTT mRNA as described in Example 1. Images were detected and analyzed under a microscope for vector genome and HTT mRNA levels.

Extensive VGs were detected in the nucleus of cells at infusion sites (thalamus). VGs were also detected in multiple different layers of motor and somatosensory cortex (mostly pyramidal neurons). Substantial VG signal was detected in the nucleus of motor and sensory cortical neurons layers I-VI after AAV1-VOYHT1 treatment.

Substantial HTT mRNA reduction was observed in cells at the infusion site (thalamus). Putamen was not significantly contained within the brain slices analyzed for ISH. ISH results demonstrated broad AAV1-VOYHT1 distribution in all NHP cortex layers and infusion sites and confirmed HTT mRNA reduction in these regions. NH results support HTT lowering in motor and somatosensory cortex and transduction of neurons in multiple layers of these regions.

xi. HTT Knockdown, VG Measurement and AAV1-VOYHT1 specific miRNA Expression in Punches from NHP Putamen

Two selected brain slabs containing the putamen from all four groups were used to collect 2 mm putamen punches. Five punches were collected from each side of one slab and 3 punches were collected from each side of the other slab, with a total of 16 punches collected from each animal. A total of 192 putamen punches were collected from all 12 animals. Samples were processed and analyzed for HTT mRNA levels and VG levels using bDNA and ddPCR, respectively. bDNA and ddPCR were carried out as described in Example 1. Samples were processed and analyzed for AAV1-VOYHT1 specific miRNA levels using deep sequencing and/or two-step stem-loop real-time quantitative PCR (RT-qPCR) approaches. For the stem-loop RT-qPCR, total RNA was purified (miRvana, catalog#AM1560, ThermoFisher Scientific) from the same punch lysate used to analyze HTT mRNA and VG, and a stem-loop oligonucleotide homologous to the AAV1-VOYHT1 specific miRNA guide strand was used to prime the reverse transcriptase reaction to generate cDNA. Then, forward and reverse primers homologous to AAV1-VOYHT1 specific miRNA and the stem-loop were used for a traditional qPCR reaction (second) step. Both the stem-loop primer and the qPCR probe set were custom-designed for the specific detection of the AAV1-VOYHT1 miRNA guide strand. All samples were blinded during the analysis. Statistical comparison of the data was performed using the one-way ANOVA with Tukey's multiple comparison test. A P value of less than 0.05 indicates a statistically significant difference.

For HTT mRNA knockdown, the relative HTT mRNA levels in all putamen punches from each AAV1-VOYHT1-treated group after normalization to the vehicle control group are presented in Table 84. Averages of 12%, 61% and 67% of HTT mRNA knockdown were achieved in putamen punches via bilateral thalamus only dosing (Group C2), and medium (Group C3) and high dose (Group C4) of bilateral putamen and thalamus dosing, respectively. Bilateral thalamus only dosing resulted in statistically significant HTT mRNA knockdown in the putamen. The percentage of punches that exhibited over 30% HTT knockdown in each group is also shown in Table 83. Both medium and high doses of bilateral putamen and thalamus dosing resulted in over 60% of putamen punches exhibiting over 30% HTT mRNA knockdown, with the high dose group having all punches exceeding 30% HTT mRNA knockdown.

TABLE 84
HTT knockdown in all putamen punches

| | | Sample | HTT mRNA relative | P value by one-way | % Punches ≥ |
|---|---|---|---|---|---|
| Group | Description | size | to vehicle (% ± stdev) | ANOVA (vs vehicle) | 30% KD |
| Group C1 | Vehicle | 48 | 100 ± 8 | — | 0 |
| Group C2 | Thalamus only | 48 | 88 ± 11 | <0.0001 | 6.3 |
| Group C3 | Medium dose | 48 | 39 ± 17 | <0.0001 | 89.6 |
| Group C4 | High dose | 48 | 33 ± 10 | <0.0001 | 100 |

The relative HTT mRNA levels analyzed from each animal in the AAV1-VOYHT1-treated groups after normalization to the vehicle control group are presented in Table 85.

TABLE 85
HTT knockdown in putamen punches averaged per animal

| | | | | P value by | P value by | P value by |
|---|---|---|---|---|---|---|
| | | | HTT mRNA | one-way | one-way | one-way |
| | | Sample | relative to vehicle | ANOVA | ANOVA | ANOVA |
| Group | Description | size | (% ± stdev) | (vs vehicle) | (vs Thalamus only) | (vs Medium dose) |
| Group C1 | Vehicle | 3 | 100 ± 6 | — | — | — |
| Group C2 | Thalamus only | 3 | 88 ± 3 | 0.1178 | — | — |
| Group C3 | Medium dose | 3 | 39 ± 8 | <0.0001 | <0.0001 | — |
| Group C4 | High dose | 3 | 33 ± 4 | <0.0001 | <0.0001 | 0.6295 |

For VG levels, the average number of vector genome copies detected in all putamen punches from each group is presented in Table 86. Averages of 21, 869, and 1211 VG copies per diploid cell were achieved in the putamen punches via bilateral thalamus only, and medium and high dose of bilateral putamen and thalamus dosing, respectively. Both medium and high doses of bilateral putamen and thalamus dosing resulted in significantly higher VG distribution to the putamen than bilateral thalamus only dosing.

TABLE 86
VG copies in all putamen punches

| | | Sample | VG copies/cell |
|---|---|---|---|
| Group | Description | size | (mean ± stdev) |
| Group C1 | Vehicle | 48 | 0.43 ± 1.33 |
| Group C2 | Thalamus only | 48 | 21.03 ± 9.05 |
| Group C3 | Medium dose | 48 | 869.3 ± 846.6 |
| Group C4 | High dose | 48 | 1211 ± 1047.0 |

The number of vector genome copies analyzed from each animal is presented in Table 87.

TABLE 87
VG copies in putamen punches averaged per animal

| | | Sample | VG copies/cell |
|---|---|---|---|
| Group | Description | size | (mean ± stdev) |
| Group C1 | Vehicle | 3 | 0.39 ± 0.53 |
| Group C2 | Thalamus only | 3 | 21.03 ± 3.72 |
| Group C3 | Medium dose | 3 | 869.3 ± 338.0 |
| Group C4 | High dose | 3 | 1211 ± 540.1 |

A Grubbs' test (Q=0.1%) was applied for removal of outliers and the VG copies/cell recalculated. Following this post-hoc statistical analysis, VG copies in putamen punches per animal were quantified as 21.0±6.5, 869.3±283.0 and 1210.8±387.3 for groups C2, C3 and C4, respectively.

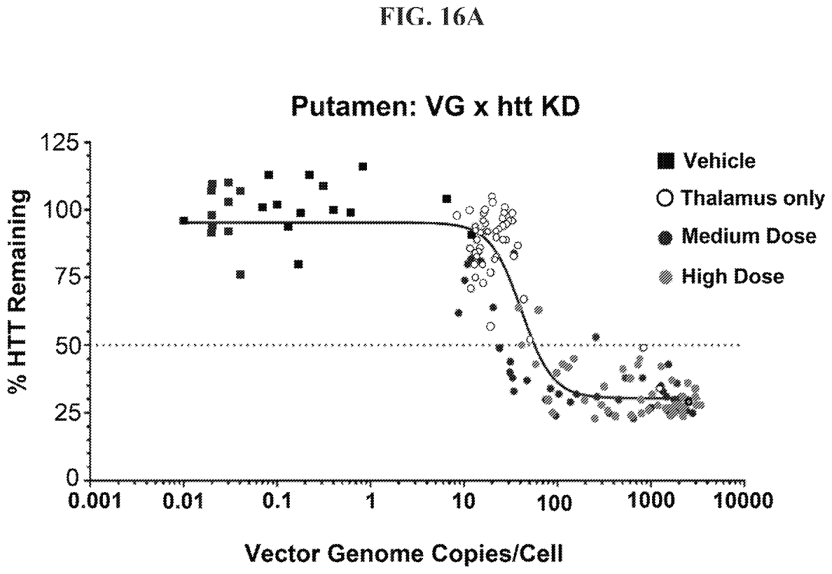

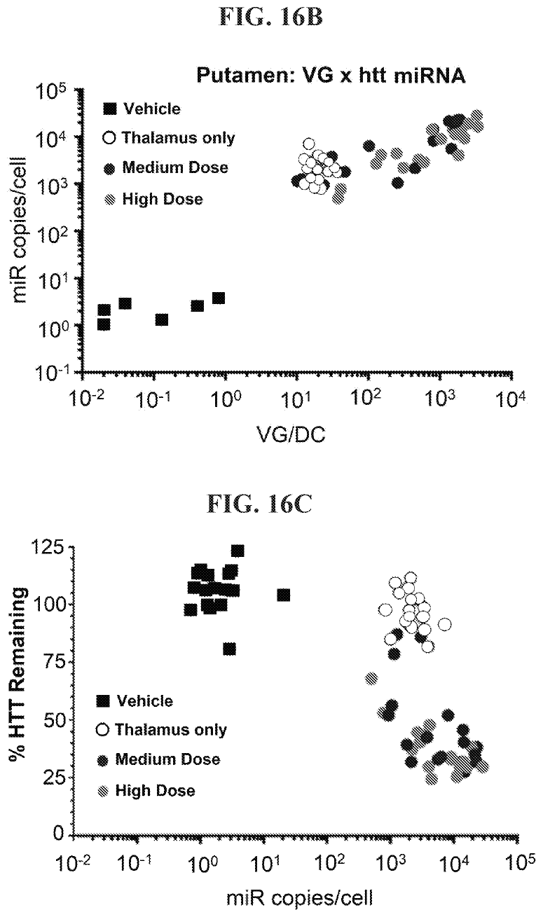

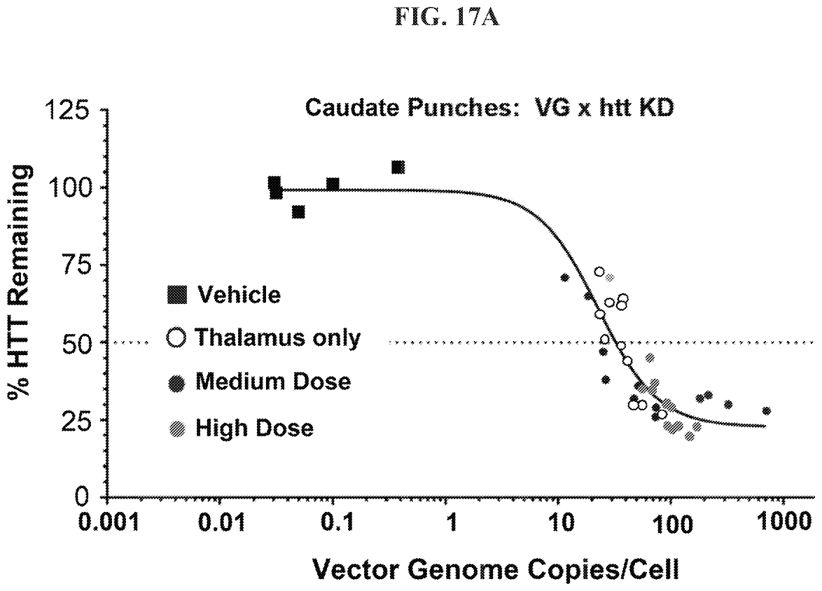

The correlation of HTT mRNA knockdown versus vector genome levels in the putamen punches is shown in FIG. 16A. The correlation curve of all putamen punches from all dosing groups results in a dose-response curve with the vehicle group at the top, thalamus only group predominantly at the top shoulder, medium dose group evenly distributed along the slope and the base, and the high dose group predominantly at the base of the curve. The EC₅₀ for HTT knockdown was calculated (Graphpad Prism, nonlinear regression 4 parameter curve fit) at approximately 40 VG per diploid cell (the range is 20-50 VG per diploid cell).

For miRNA analyses, the average number of AAV1-VOYHT1 specific miRNA copies per cell and corresponding average VG copies per cell, HTT mRNA levels relative to control, and AAV1-VOYHT1 specific miRNA per VG calculation averaged per animal are presented in Table 88. These analyses were performed using a subset of putamen punches, and thus, values presented in Table 88 refer to data from 6 putamen punches per animal (3 per hemisphere) for a total of 72 total samples.

TABLE 88
AAV1-VOYHT1 specific miRNA expression in putamen punches averaged per animal

| | | | miRNA | | HTT mRNA | |
|---|---|---|---|---|---|---|
| | | Sample | copies/cell | VG copies/cell | relative to vehicle | HTT miRNA/VG |
| Group | Description | size | (mean ± stdev) | (mean ± stdev) | (% ± stdev) | (mean ± stdev) |
| Group C1 | Vehicle | 3 | 2.9 ± 4.44 | 0.08 ± 0.21 | 100 ± 9 | 15 ± 32 |
| Group C2 | Thalamus only | 3 | 2283 ± 1495 | 21 ± 7 | 91 ± 8 | 128 ± 107 |
| Group C3 | Medium dose | 3 | 8808 ± 7955 | 793 ± 773 | 44 ± 18 | 44 ± 55 |
| Group C4 | High dose | 3 | 8869 ± 7568 | 1258 ± 1118 | 35 ± 10 | 11 ± 7 |

The correlation of AAV1-VOYHT1 specific miRNA expression versus vector genome levels in all putamen punches from each treatment group (r=0.8606, p<0.001)) is shown in FIG. 16B. Enhanced VG biodistribution corresponded to increased AAV1-VOYHT1 specific miRNA expression in AAV1-VOYHT1-treated group.

The correlation of AAV1-VOYHT1 specific miRNA expression versus vector HTT mRNA lowering in all putamen punches from each treatment group (r=−0.6788, p<0.0001) is shown in FIG. 16C. Increased AAV1-VOYHT1 specific miRNA expression corresponded to enhanced HTT mRNA lowering AAV1-VOYHT1-treated group.

Together, thalamus-only dosing resulted in more modest VG biodistribution, AAV1-VOYHT1 specific miRNA expression, and HTT mRNA lowering in putamen. Combined putamen and thalamus dosing resulted in greater VG biodistribution. AAV1-VOYHT1 specific miRNA expression, and robust HTT mRNA lowering in putamen relative to thalamus only dosing. Finally, AAV1-VOYHT1 specific miRNA expression correlates with VG biodistribution and HTT mRNA lowering.

xii. HTT Knockdown, VG Measurement and AAV1-VOYHT1 Specific miRNA Expression in Punches from NHP Caudate

A selected brain slab containing the caudate from all four groups was used to collect 2 mm caudate punches. Two punches were collected from each side of the slab, with a total of 4 punches collected from each animal. A total of 48 caudate punches was collected from all 12 animals. Samples were processed and analyzed for HTT mRNA levels and VG levels using tDNA and ddPCR, respectively. hDNA and ddPCR were carried out as described in Example 1. Samples were processed and analyzed for AAV1-VOYHT1 specific miRNA levels using deep sequencing and/or two-step stein-loop real-time quantitative PCR (RT-qPCR) approaches. For the stem-loop RT-qPCR, total RNA was purified (miRvana, catalog#AM1560, ThermoFisher Scientific) from the same punch lysate used to analyze HTT mRNA and VG, and a stem-loop oligonucleotide homologous to the AAV1-VOYHT1 specific miRNA guide strand was used to prime the reverse transcriptase reaction to generate cDNA. Then, forward and reverse primers homologous to AAV1-VOYHT1 specific miRNA and the stem-loop were used fora traditional qPCR reaction (second) step. Both the stem-loop primer and the qPCR probe set were custom-designed for the specific detection of the AAV1-VOYHT1 miRNA guide strand. All samples were blinded during the analysis. Statistical comparison of the data was performed using the one-way ANOVA with Tukey's multiple comparison test. A P value of less than 0.05 indicates a statistically significant difference.

For HTT mRNA knockdown, the relative HTT mRNA levels in all caudate punches from each AAV1-ATOYHT1-treated group after normalization to the vehicle control group are presented in Table 89. Averages of 51%, 61% and 68% of HTT mRNA knockdown were achieved in caudate punches via bilateral thalamus only dosing (Group C2), and medium (Group C3) and high dose (Group C4) of bilateral putamen and thalamus dosing, respectively. Bilateral thalamus only dosing caused robust and significant HTT mRNA knockdown (by 51%) in the caudate punches. The percentage of punches that exhibited over 30% HTT knockdown in each group is also shown in Table 88. All three dosing groups (bilateral thalamus only dosing, medium and high dose of bilateral putamen and thalamus dosing) had 92% of caudate punches achieving at least 30% HTT mRNA knockdown.

TABLE 89
HTT knockdown in all caudate punches

| | | Sample | HTT mRNA Relative | P value by one-way | % Punches ≥ |
|---|---|---|---|---|---|
| Group | Description | size | to Vehicle (% ± stdev) | ANOVA (vs vehicle) | 30% KD |
| Group C1 | Vehicle | 12 | 100 ± 6 | — | 0 |
| Group C2 | Thalamus only | 12 | 49 ± 16 | <0.0001 | 92 |
| Group C3 | Medium dose | 12 | 39 ± 15 | <0.0001 | 92 |
| Group C4 | High dose | 12 | 32 ± 15 | <0.0001 | 92 |

The relative HTT mRNA levels analyzed from each animal in the AAV1-VOYHT1-treated groups after normalization to the vehicle control group are presented in Table 90.

TABLE 90
HTT knockdown in caudate punches averaged per animal

| | | | | P value by | P value by | P value by |
|---|---|---|---|---|---|---|
| | | | HTT mRNA | one-way | one-way | one-way |
| | | Sample | Relative to Vehicle | ANOVA | ANOVA | ANOVA |
| Group | Description | size | (% ± stdev) | (vs vehicle) | (vs Thalamus only) | (vs Medium dose) |
| Group C1 | Vehicle | 3 | 100 ± 6 | — | — | — |
| Group C2 | Thalamus only | 3 | 49 ± 13 | 0.0012 | — | — |
| Group C3 | Medium dose | 3 | 39 ± 11 | 0.0004 | 0.6889 | — |
| Group C4 | High dose | 3 | 32 ± 9 | 0.0002 | 0.2786 | 0.8347 |

For VG levels, the average number of vector genome copies detected in all caudate punches from each group is presented in Table 91. An average of 44, 146, and 99 vector genome copies per diploid cell was achieved in the caudate punches via bilateral thalamus-only dosing, medium and high doses of bilateral putamen and thalamus dosing, respectively.

TABLE 91
VG copies in all caudate punches

| | | Sample | VG copies/cell |
|---|---|---|---|
| Group | Description | size | (mean ± stdev) |
| Group C1 | Vehicle | 12 | 0.12 ± 0.15 |
| Group C2 | Thalamus only | 12 | 44.18 ± 22.86 |
| Group C3 | Medium dose | 12 | 146.2 ± 200.9 |
| Group C4 | High dose | 12 | 99.22 ± 45.01 |

The number of vector genome copies analyzed from each animal is presented in Table 92.

TABLE 92
VG copies in caudate punches per animal

| | | Sample | VG copies/cell |
|---|---|---|---|
| Group | Description | size | (mean ± stdev) |
| Group C1 | Vehicle | 3 | 0.10 ± 0.05 |
| Group C2 | Thalamus only | 3 | 44.18 ± 10.16 |
| Group C3 | Medium dose | 3 | 146.2 ± 182.9 |
| Group C4 | High dose | 3 | 99.22 ± 29.22 |

A Grubbs' test (Q=0.1%) was applied for removal of outliers and the VG copies/cell in caudate recalculated. Following this post-hoc statistical analysis, VG copies in caudate punches per animal were quantified as 44.2±10.2, 107.4±116.0 and 99.2±29.2 for groups C2, C3 and C4, respectively.

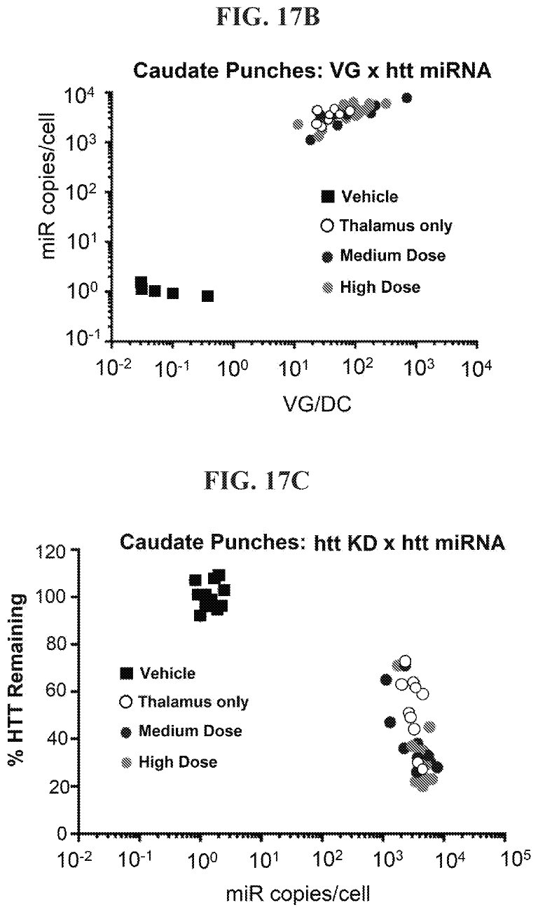

The correlation of HTT mRNA knockdown versus vector genome levels in the caudate punches is shown in FIG. 17A. The correlation curve of all caudate punches from all dosing groups results in a dose-response curve with the vehicle group at the top, and all other dosing groups dispersed along the slope and beginning of the base of the curve. The EC₅₀ for HTT knockdown was calculated (Graphpad Prism, nonlinear regression 4 parameter curve fit) at approximately 23 VG per diploid cell (the range is 20-50 VG per diploid cell).

For miRNA analyses, the average number of AAV1-VOYHT1 specific miRNA copies per cell and corresponding average VG copies per cell, HTT mRNA levels relative to control, and AAV1-VOYHT1 specific miRNA per VG calculation averaged per animal are presented in Table 93. These analyses were performed using a subset of caudate punches, and thus, values presented in Table 93 refer to data from 4 putamen punches per animal (2 per hemisphere) for a total of 48 total samples.

TABLE 93
AAV1-VOYHT1 specific miRNA expression in canduate punches averaged per animal

| | | | | | HTT mRNA | |
|---|---|---|---|---|---|---|
| | | | miRNA | | relative to | |
| | | Sample | copies/cell | VG copies/cell | vehicle | HTT miRNA/VG |
| Group | Description | size | (mean ± stdev) | (mean ± stdev) | (% ± stdev) | (mean ± stdev) |
| Group C1 | Vehicle | 3 | 1.5 ± 0.53 | 0.05 ± 0.11 | 100 ± 6 | 10 ± 19 |
| Group C2 | Thalamus only | 3 | 3535 ± 1050 | 44 ± 10 | 49 ± 13 | 90 ± 35 |
| Group C3 | Medium dose | 3 | 3730 ± 1944 | 146 ± 183 | 39 ± 11 | 62 ± 55 |
| Group C4 | High dose | 3 | 4468 ± 1356 | 99 ± 29 | 32 ± 9 | 51 ± 20 |

The correlation of AAV1-VOYHT1 specific miRNA expression versus vector genome levels in all caudate punches from each treatment group (r=0.6782, p<0.0001) is shown. in FIG. 17B, Enhanced VG biodistribution corresponded to increased AAV1-VOYHT1 specific miRNA expression in AAV1-VOYHT1-treated group. A Grubbs' test (Q=0.1%) was used to detect significant outliers when vector genome level data from animal groups in Dose Optimization Study III were combined with those from Dose Optimization Study I, as above. A positive relationship between AAV1-VOYHT1 specific miRNA expression versus vector genome levels in caudate punches from each treatment group remained following the removal of one outlier value from the correlation analysis shown in FIG. 17B (r=0.7452, p<0.001). Following removal of all outliers, VG copies/cell (mean±stdev) in caudate punches for Groups A2, A3, A5 and C3 to 1.8±0.5. 10.7±10.3, 0.3±0.2 and 107.4±116.0 VG copies/cell, respectively.

The correlation of AAV1-VOYHT1. specific miRNA expression versus vector HTT mRNA lowering in all caudate punches from each treatment group (r =−0.8798, p<0.0001) is shown in FIG. 17C. Increasing AAV1-VOYHT1 specific miRNA expression corresponded to enhanced HTT mRNA lowering AAV1-VOYHT1-treated group.

Together, thalamus-only dosing resulted in significant VG biodistribution, significant AAV1-VOYHT1 specific miRNA expression, and substantial HTT mRNA lowering in caudate. Combined putamen and thalamus dosing resulted in greater VG biodistribution, AAV1-VOYHT1 specific miRNA expression, and robust HTT mRNA lowering in caudate compared with thalamus-only dosing. Finally, AAV1-VOYHT1 specific miRNA expression correlates with VG biodistribution and HTT mRNA lowering.

xiii. HTT Knockdown and VG Measurement in Punches from NHP Thalamus

A selected brain slab containing the thalamus from all four groups was used to collect 2 mm thalamus punches, Five punches were collected from each side of the slab, with a total of 10 punches collected from each animal. A total number of 120 thalamus punches were collected from all 12 animals. Samples were processed and analyzed for HTT mRNA levels and VG levels using bDNA and ddPCR, respectively. bDNA and ddPCR were carried out as described in Example 1. All samples were blinded during the analysis. Statistical comparison of the data was performed using the one-way ANOVA with Tukey's multiple comparison test. A P value of less than 0.05 indicates a statistically significant difference.

For HTT mRNA knockdown, the relative HTT mRNA levels in all thalamus punches from the AAV1-VOYHT1-treated groups after normalization to the vehicle control group are presented in Table 94. Averages of 76%, 76% and 73% of HTT mRNA knockdown were achieved in the thalamus punches via bilateral thalamus only dosing (Group C2), and medium (Group C3) and high dose (Group C4) of bilateral putamen and thalamus dosing, respectively. The percentage of punches that exhibited over 30% HTT knockdown in each group is also shown in Table 94. 100% of the thalamus punches achieved at least 30% of HTT mRNA KD for all three dosing groups.

TABLE 94
HTT knockdown in all thalamus punches

| | | Sample | HTT mRNA relative | P value by one-wav | % Punches ≥ |
|---|---|---|---|---|---|
| Group | Description | size | to vehicle (% ± stdev) | ANOVA (vs vehicle) | 30% KD |
| Group C1 | Vehicle | 30 | 100 ± 8 | — | 0 |
| Group C2 | Thalamus only | 30 | 24 ± 5 | <0.0001 | 100 |
| Group C3 | Medium dose | 30 | 24 ± 9 | <0.0001 | 100 |
| Group C4 | High dose | 30 | 27 ± 5 | <0.0001 | 100 |

The relative HTT mRNA levels analyzed from each animal in the AAV1-VOYHT1-treated groups after normalization to the vehicle control group are presented in Table 95.

TABLE 95
HTT knockdown in thalamus punches averaged per animal

| | | | HTT mRNA | P value by | P value by | P value by |
|---|---|---|---|---|---|---|
| | | | Relative to | one-wav | one-way | one-way |
| | | Sample | Vehicle | ANOVA | ANOVA | ANOVA |
| Group | Description | size | (% ± stdev) | (vs vehicle) | (vs Thalamus only) | (vs Medium dose) |
| Group C1 | Vehicle | 3 | 100 ± 3 | — | — | — |
| Group C2 | Thalamus only | 3 | 24 ± 4 | <0.0001 | — | — |
| Group C3 | Medium dose | 3 | 24 ± 5 | <0.0001 | 0.9995 | — |
| Group C4 | High dose | 3 | 27 ± 2 | <0.0001 | 0.7505 | 0.8081 |

For VG levels, the average number of vector genome copies detected in all thalamus punches from each group is presented in Table 96. Similar levels of vector genome copies in all 3 treatment groups were observed. Averages of 2015, 1704, 2747 vector genome copies per diploid cell were achieved in the thalamus punches via bilateral thalamus-only dosing, medium and high dose of bilateral putamen and thalamus dosing, respectively.

TABLE 96
VG copies in all thalamus punches

| | | Sample | VG copies/cell |
|---|---|---|---|
| Group | Description | size | (mean ± stdev) |
| Group C1 | Vehicle | 30 | 0.13 ± 0.27 |
| Group C2 | Thalamus only | 30 | 2015 ± 1088 |
| Group C3 | Medium dose | 30 | 1704 ± 741.9 |
| Group C4 | High dose | 30 | 2747 ± 896.1 |

The number of vector genome copies analyzed from each animal is presented in Table 97.

TABLE 97
VG copies in thalamus punches averaged per animal

| | | Sample | VG copies/cell |
|---|---|---|---|
| Group | Description | size | (mean ± stdev) |
| Group C1 | Vehicle | 3 | 0.13 ± 0.07 |
| Group C2 | Thalamus only | 3 | 2015 ± 310.9 |
| Group C3 | Medium dose | 3 | 1704 ± 467.3 |
| Group C4 | High dose | 3 | 2747 ± 691.2 |

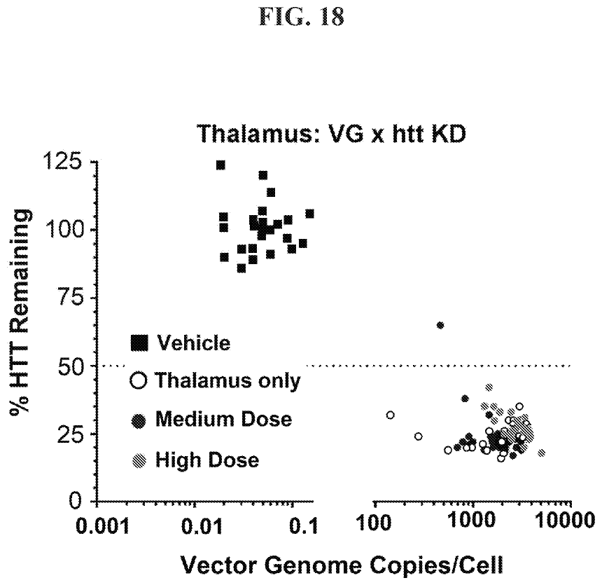

The correlation of HTT mRNA knockdown versus vector genome levels in the thalamus punches is shown in FIG. 18. All dosing groups achieved similar vector genome copies per cell and similar knockdown efficiency in punches from the thalamus. The correlation plot of all thalamus punches from all dosing groups shows the vehicle group in the top left and all other dosing groups mostly overlapping one another in the far right of the base. The EC₅₀ calculations were ambiguous due to the presence of predominantly fully positive and negative populations.

In summary, the punch analyses from the putamen, caudate, and thalamus reveal that substantial HTT mRNA knockdown was achieved at the infusion sites (putamen and thalamus) as well as in the caudate in all three dosing groups (thalamus-only dosing, and medium and high doses of bilateral putamen and thalamus dosing). Further, vector genome levels correlate well with HTT mRNA knockdown in the putamen, caudate and thalamus with evidence for a plateau in knockdown at high vector genome levels.

xiv. Clinical Signs and Histopathology

In 7 out of 9 NHPs that received AAV1-VOYHT1, no clinical signs or limb findings were observed post-infusion. In the other two NHPs, shortened steps and slight limb finding were observed. However, no histopathological changes were seen which would account for, or correlate with these clinical signs. Histopathologic findings associated with catheter tip and/or track were expected due to the surgical procedure, but none resulted in any specific clinical sign. Minimal findings at the thalamic sites of infusion were expected and included gliosis, neuronal degeneration, glial cell vacuolation and mononuclear cell infiltration that were slightly more widespread than in putamen. None was expected to result in any clinical signs. Edema was only observed adjacent to the catheter track, suggesting that volumes were well-tolerated. No evidence of detrimental effect on neurons of the somatosensory or motor cortices was seen in any group. These findings suggest that the no-observed-adverse-effect-level (NOAEL) is, at a minimum, AAV1-VOYHT1 administered at the high dose via putamen and thalamus infusion (see Group C4).

### Example 4

### Formulation Optimization

Initial formulation screening identified a Phosphate/Sucrose/NaCl formulation (2.7 mM Na Phosphate (dibasic), 1.54 mM K Phosphate (mono), 155 mM NaCl, and 5% (w/v) Sucrose at pH 7.2, 450 mOsm/kg) as an acceptably stable formulation for the AAV1-VOYHT1 vector. High salt formulations were also identified as stabilizing.

The formulation was further optimized for excipients, Na/K ratios, pH, and osmolality while adjusting for factors suitable for CNS administration. Three solutions that may be used to formulate the AAV1-VOYHT1 vector are presented in Table 98.

TABLE 98
Formulations for AAV1-VOYHT1 vector

| Formulation 1 | Formulation 2 | Formulation 3 |
|---|---|---|
| 10 mM Sodium Phosphate | 10 mM Tris Base | 10 mM Tris Base |
| 1.5 mM Potassium Phosphate | 6.25 mM HCl | 8.95 mM HCl |
| 95 mM Sodium Chloride | 1.5 mM Potassium Chloride | 1.5 mM Potassium Chloride |
| 7% (w/v) Sucrose | 100 mM Sodium Chloride | 100 mM Sodium Chloride |
| 0.001% (w/v) Pluronic ® F-68 | 7% (w/v) Sucrose | 7% (w/v) Sucrose |
| pH 7.4 ± 0.2 at 5° C. | 0.001% (w/v) Pluronic ® F-68 | 0.001% (w/v) Pluronic ® F-68 |
| | pH 8.0 ± 0.2 at 5° C. | pH 7.5 ± 0.2 at 5° C. |

The concentration of the AAV1-VOYHT1 vector to be formulated in the above identified solutions is about 2.7e13 vg/mL, but the concentration may be increased up to 5e13 vg/ml. High concentration AAV1-VOYHT1 vectors were shown to be difficult to stabilize in the absence of aggregation. Analysis of a formulation screen indicated that an increase in sucrose level generally improves vector stability and prevents aggregation. Sucrose levels from about 5% to 9% provided good stability for the AAV1-VOYHT1 vector, with the optimal concentration at about 7% sucrose for the tested vector and desired formulation. concentration. The level of sucrose use may be limited by physiological osmolality. Furthermore, higher osmolality and/or more NaCl were shown to be favorable for vector stability.

### Example 5

### Administration of AAV1-VOYHT1 to HD Patients

AAV1-VGYHT1 vectors formulated in an appropriate formulation identified in Example 4 are administered into a Stage 1 HD patient via bilateral parenchymal infusion to the putamen and thalamus using MRI-guided convection enhanced delivery (CED). The concentration of AAV1-VOYHT1 vectors in the formulated solution to be infused is between 2.7e12 to 2.7e13 vg/mL. The volumes of AAV1-VOYHT1 infused to the putamen and thalamus are 300-1500 μL/hemisphere and 1300-2500 μL/hemisphere, respectively. The doses administered to the putamen and the thalamus are 8e1l to 4e13 vg/hemisphere and 3.5e12 to 6.8e13 vg/hemisphere, respectively. The total dose administered to the patient is about 8.6e12 to 2e14 vg. The AAV1-VOYHT1 treatment results in significant reduction in HTT mRNA levels in the striatum and cortex of the patient.

### Example 6

### Minimal Effective Dose (MED) Study

i. Study design

As an extension of Example 1 (Dose Optimization Study I) above, this study interrogates the pharmacological activity profile of low doses of AAV1-VOYHT1; SEQ ID NO for VOYHT1: 1352 formulated in an aqueous solution containing 7% sucrose, within striatum, cortex, and thalamus of rhesus macaques, to identify a minimally effective dose or minimum efficacious dose (MED), or minimally effective concentration (MEC), fir extrapolation to a clinical dosing paradigm. MED/MEC safety margins are assessed toward specific development of a therapeutic for the treatment of early symptomatic and/or prodromal Huntington's disease (HD). Aims of the study include achieving target HTT lowering (mRNA knockdown) in brain regions, e.g. striatum, cortex, and thalamus and, in particular, cortical neurons with a MED.

To guide MED study parameters and to evaluate safety and biodistribution of VY-HTT01 a No Observed Adverse Effect Level (NOEFL) dose is evaluated and identified in adult rhesus macaques (Macaw mulatta). Animals (N=48) are pre-screened for inclusion in the NOEFL evaluation using capsid specific anti-AAV1 antibodies. Test article containing AAV1-VOYHT1, or a vehicle control is delivered by the intended route of administration for clinical trial, e.g., bilateral intraparenchymal infusions into the putamen (posterior) and thalamus (prefrontal), using magnetic resonance imaging (MRI)-guided convection-enhanced delivery (CED) and a stereotactic device. Doses of test article to be tested range from 0 vg/animal (vehicle) to 2.24×10¹³ vg/mL (AAV1-VOYHT1). Together, the three AAV1-VOYHT1 doses (low-, mid-, and high-dose) are 2.24×10¹² vg/animal (4.2×10¹¹ vg/putamen, 7.0×10¹¹ vg/thalamus), 6.44×10¹² vg/mL (1.22×10¹² vg/putamen, 2.0×10¹² vg/thalamus), and 2.24e13 vg/mL (4.2×10¹² vg/putamen, 7.0×10¹² vg/thalamus). The infusion parameters for putamen are: 1-5 μL/min (rate); 10-28 min (duration) for a sum of 54 min; and 16-84 μL (volume) for a sum of 150 μL. The infusion parameters for thalamus are: 1-5 μL/min (rate); 15-50 min (duration) for a sum of 90 min; and 25-150 μL for a sum of 250 μL. Clinical signs are periodically evaluated across the lifespan. Animals are euthanized 5, 13, 26, or 53 weeks after dosing, and nervous and other tissues are collected for post-mortem analysis. Biodistribution of viral genomes, micro RNA levels, reduction in HTT mRNA, reduction in HTT protein, and histology of the brain are evaluated across timepoints.

For the MED study, a total of 12 animals is used. Each animal is assigned to 1 of 4 treatment groups as summarized in Table 99, Animals receive bilateral intraparenchymal infusions of the test article containing AAV1-VOYHT1 or a vehicle control into the putamen (posterior) and thalamus (prefrontal) using magnetic resonance imaging (MRI)-guided convection-enhanced delivery (CED). A Functional Observation Battery (FOB) evaluation focusing on neurological status is carried out prior to dosing with test article or vehicle. The same FOB is performed at post-infusion time-points, e.g., 7 and 93 days, as well as weekly during the lifespan if warranted. Animals are euthanized 13 weeks after dosing, and tissues are collected for post-mortem analyses.

TABLE 99
Dilutions and dosing solutions

| | | | Target site | | |
|---|---|---|---|---|---|
| | | | per hemisphere | | |
| | Number | Dosing | (vol. and total vg) | | |
| | of | Titer | Putamen | Thalamus | Total dose |
| Test article | animals | (vg/ml) | (150 μL) | (250 μL) | (vg/animal) |
| Vehicle | 1 M/2 F | 0 | 0 | 0 | 0 |
| VY-HTT01 | 2 M/1 F | 1.9e11 | 2.85e10 | 4.75e10 | 1.5e11 |
| | 1 M/2 F | 5.7e11 | 8.6e10 | 1.4e11 | 4.5e11 |
| | 2 M/1 F | 2.8e12 | 4.2e11 | 7e11 | 2.2e12 |

For post-mortem analysis of tissue, biodistribution of vector genomes is assessed in 22 sections of brain, laser-captured cortical neurons, and in highly perfused tissues (e.g., heart, lung, liver, spleen, kidney, and draining lymph node). MicroRNA and mRNA are also assessed in 22 sections of brain including those sampled from striatum (caudate and putamen), thalamus, and cortex. mRNA is measured in laser-captured cortical neurons. HTT protein is measured in striatum, thalamus, cortex and biofluids, e.g., cerebrospinal fluid (CSF). Histology is performed in brain and highly perfused tissues possessing at least 50 copies/μg genomic DNA.

Human equivalent doses can be calculated based on the results of this study.

Detailed methods are as described above in Example 1.

### Example 7

### HTT Protein and mRNA Evaluation in NHP Putamen, Thalamus, and Caudate

i. Study Design

As an extension of Example I (Dose Optimization Study I) and of Example 3 (Dose Optimization Study III), described in detail above, this study was designed to measure HTT protein and mRNA levels in tissue punches from putamen, caudate and thalamus five weeks following bilateral combined intraputaminal and intrathalamic infusion of different doses of AAV1-VOYHT1 (herein referred to as AAV1-VOYHT1 or VY-HTT01, SEQ ID NO for VOYHT1: 1352) in non-human primate (NHP) rhesus macaque (Macaca mulatto).

This study involved five groups of animals selected from Examples 1. and 3 (above), in order to span an approximately 100-fold range of total VY-HTT01, with approximately 10-fold dose increments, Animal study groups, including detailed dosing parameters, are presented in Table 100.

TABLE 100
Study design

| | | | | | Dosing | Total |
|---|---|---|---|---|---|---|
| | Number of | | Volume μL/side) | | titer | dose |
| Group | animals | Description | Putamen | Thalamus | (VG/mL) | (VG) |
| Group A6 | 3 | Vehicle | Left: 100 | Left: 150 | 0.0 | 0.0 |
| | | | Right: 150 | Right: 250 | | |
| Group A5 | 3 | Mid vol, low conc | 100 | 150 | 2.7e11 | 1.35e11 |
| Group A2 | 3 | Mid vol, high conc | 100 | 150 | 2.7e12 | 1.35e12 |
| Group C1b | 3 | Vehicle | 150 | 250 | 0.0 | 0.0 |
| Group C4 | 3 | High vol, high conc | 150 | 250 | 2.2e13 | 1.78e13 |

Apart from quantitation of HTT protein by UPLC-MS/MS (Ultra performance liquid chromatography-mass spectrometry and liquid chromatography-tandem mass spectrometry), or LC-MS/MS, as outlined below, detailed methods are as described above in Example 1.

ii. LC-MS/MS for Quantitation of HTT Protein

Monkey brain tissue punches from putamen, caudate, and thalamus after bilateral, combined intraspinal and intrathalamic injection of VY-HTT01 doses, as described above in Example 1, were weighed and pulverized into a dry tissue powder for subsequent quantitation of HTT protein by UPLC-MS/MS, or LC-MS/MS. HTT protein levels were measured based on a representative HTT peptide derived from a trypsin digestion procedure, and normalized to the levels of actin. Briefly, pulverized brain tissue powders were homogenized in tissue protein extraction reagent. The homogenates were centrifuged and then supernatants (lysates) were reduced, denatured, and alkylated with iodoacetamide. The alkylated samples were treated with trypsin to generate a HTT peptide. Concentrations of the HTT peptide were measured in the digested samples by LC-MS/MS. The calibration curves of the LC-MS/MS method were prepared with synthetic peptides, Corresponding mass-shifted, stable isotope-labeled peptides were employed as internal standards. A high resolution SCIEX Triple TOF 6600 LC-MS/MS system with Analyst and MultiQuant software was used for quantitation. The signature peptide levels measured in the digested samples (ng/mL) were converted to protein concentrations by molecular weight. The HTT protein concentrations were corrected for sample work up and reported in ng/mg actin.

ii. Putamen HTT Protein and mRNA Levels

To evaluate HTT protein and mRNA levels in the putamen, 4 tissue punches were collected from the putamen in the right hemisphere, resulting in a total of 12 punches per VY-HTT01 group, and 24 punches for two vehicle groups. As described above, putamen tissue punch powders were used for HTT and actin protein analysis by LC-MS/MS. HTT protein levels were normalized to the levels of actin, and then further normalized to the vehicle group. The average relative remaining putamen HTT protein and relative putamen HTT protein knockdown (KD), expressed as a percentage of vehicle (veh), as well as p-values generated by one-way ANOVA with Tukey's multiple comparisons, are presented in Table 101.

TABLE 101
HTT protein lowering in NHP putamen

| | | | Relative | | P value by | P value by | P value by |
|---|---|---|---|---|---|---|---|
| | | Total | remaining | | one-way | one-way | one-way |
| | Number of | dose | HTT protein | HTT protein KD | ANOVA | ANOVA | ANOVA |
| Group | animals | (VG) | (% of veh ± stdev) | (% of veh ± stdev) | (vs Group A5) | (vs Group A2) | (vs Group C4) |
| Group A6 | 3 | 0.0 | 100 ± 6 | 0 ± 6 | 0.0265 | <0.0001 | <0.0001 |
| Group C1b | 3 | 0.0 | | | | | |
| Group A5 | 3 | 1.35e11 | 81 ± 12 | 19 ± 12 | — | 0.0002 | 0.0001 |
| Group A2 | 3 | 1.35e12 | 38 ± 2 | 62 ± 2 | — | — | 0.9966 |
| Group C4 | 3 | 1.78e13 | 37 ± 11 | 63 ± 11 | — | — | — |

A significant and dose-dependent HTT protein reduction was achieved in the putamen after bilateral intraputaminal and intrathalamic infusion of different total doses of VY-HTT01. Specifically, on average, there was 63%, 62%, and 19% HTT protein KD for group C4 (1.78×10¹³ VG, or VG per animal), group A2 (1.35×10¹² VG), and group A5 (1.35×10¹¹ VG), respectively, relative to the vehicle group (groups A6 and C1b).

HTT protein KD was statistically significant (P<0.05 by one-way ANOVA with Tukey's multiple comparisons test) at all doses of VY-HTT01. versus vehicle, with the highest dose of 1.78×10¹³ VG, or VG per animal (group C4), resulting in statistically greater HTT protein KD (P<0.001 by one-way ANOVA with Tukey's multiple comparisons test) than was achieved with a total dose of 1.35×10¹¹VG per animal (group A5). In addition, the second highest dose of 1.35×10¹² VG per animal (group A2) resulted in statistically greater HTT protein KD (P<0.001 by one-way ANOVA with Tukey's multiple comparisons test) than the lowest dose (1.35×10¹¹ VG per animal) evaluated. There was no statistically significant difference in HTT protein KD between the two highest total doses (1.78×10¹³ and 1.35×10¹² VG per animal) tested.

HTT mRNA levels in the putamen were also measured by the bDNA assay, as described in Example 1, in the same putamen punches used for HTT protein quantitation, using the specific probe set against rhesus HTT, TBP, AARS and XPNPEP1. HTT mRNA levels were normalized to the geometric mean of the three housekeeping genes TBP, AARS and XPNPEP1, and then further normalized to the vehicle group. The average relative remaining putamen HTT mRNA and relative putamen HTT mRNA knockdown (KD), expressed as a percentage of vehicle (veh), as well as p-values generated by one-way ANOVA with Tukey's multiple comparisons, are presented in Table 102.

TABLE 102
HTT mRNA lowering in NHP putamen

| | | | Relative | | P value by | P value by | P value by |
|---|---|---|---|---|---|---|---|
| | | Total | remaining | | one-way | one-way | one-way |
| | Number of | dose | HTT mRNA | HTT mRNA KD | ANOVA | ANOVA | ANOVA |
| Group | animals | (VG) | (% of veh ± stdev) | (% of veh ± stdev) | (vs Group A5) | (vs Group A2) | (vs Group C4) |
| Group A6 | 3 | 0.0 | 100 ± 4 | 0 ± 4 | 0.004 | <0.0001 | <0.0001 |
| Group C1b | 3 | 0.0 | | | | | |
| Group A5 | 3 | 1.35e11 | 77 ± 7 | 23 ± 7 | — | 0.0007 | 0.0006 |
| Group A2 | 3 | 1.35e12 | 43 ± 14 | 57 ± 14 | — | — | 0.9995 |
| Group C4 | 3 | 1.78e13 | 42 ± 3 | 58 ± 3 | — | — | — |

A significant and dose-dependent HTT mRNA reduction was achieved in the putamen after bilateral intraputaminal and intrathalamic infusion of different total doses of VY-HTT01. Specifically, on average, there was 58%, 57% and 23% HTT mRNA KD for group C4 (total 1.78×10¹³ VG per animal), group A2 (total 1.35×10¹² VG per animal) and group A5 (total 1.35×10¹¹ VG per animal), respectively, relative to the vehicle groups (groups A6 and C1b). The HTT mRNA KD was statistically significant (P<0.01 by one-way ANOVA with Tukey's multiple comparisons test) at all doses of VY-HTT01 versus vehicle, with the highest dose of 1.78×10¹³ VG per animal resulting in statistically greater HTT mRNA KD (P<0.001 by one-way ANOVA with Tukey's multiple comparisons test) than was achieved with a total dose of 1.35×10¹¹ VG per animal. In addition, the second highest dose of 1.35×10¹² VG per animal resulted in statistically greater HTT mRNA KD (P<0.001 by one-way ANOVA with Tukey's multiple comparisons test) than the lowest dose (1.35×10¹¹ VG per animal) evaluated. There was no statistically significant difference in HTT mRNA KD between the two highest total doses (1.35×10¹² and 1.78×10¹³ VG per animal) evaluated.

iv. Caudate HTT Protein and mRNA Levels

To evaluate HTT protein and mRNA levels in the caudate, 4 punches were collected from each animal (2 per hemisphere) for HTT and actin protein analysis by LC-MS/MS, resulting in a total of 12 punches per VY-HTT01 treatment group, and 24 punches for two vehicle groups. HTT protein levels were normalized to the levels of actin, and then further normalized to the vehicle group. The average relative remaining caudate HTT protein and relative caudate HTT protein knockdown (KD), expressed as a percentage of vehicle (veh), as well as p-values generated by one-way ANOVA with Tukey's multiple comparisons, are presented in Table 103.

TABLE 103
HTT protein lowering in NHP caudate

| | | | Relative | | P value by | P value by | P value by |
|---|---|---|---|---|---|---|---|
| | | Total | remaining | | one-way | one-way | one-way |
| | Number of | dose | HTT protein | HTT protein KD | ANOVA | ANOVA | ANOVA |
| Group | animals | (VG) | (% of veh ± stdev) | (% of veh ± stdev) | (vs Group A5) | (vs Group A2) | (vs Group C4) |
| Group A6 | 3 | 0.0 | 100 ± 8 | 0 ± 8 | 0.8702 | 0.204 | 0.0004 |
| Group C1b | 3 | 0.0 | | | | | |
| Group A5 | 3 | 1.35e11 | 94 ± 12 | 6 ± 12 | — | 0.649 | 0.0037 |
| Group A2 | 3 | 1.35e12 | 84 ± 16 | 16 ± 16 | — | — | 0.0256 |
| Group C4 | 3 | 1.78e13 | 54 ± 9 | 46 ± 9 | — | — | — |

A significant and dose-dependent T protein reduction was achieved in the caudate after bilateral intraputaminal and intrathalamic infusion of different total doses of VY-HTT01. Specifically, on average, there was 46%, 16% and 6% HTT protein KD for group C4 (total 1.78×10¹³ VG per animal), group A2 (total 1.35×10¹² VG per animal) and group 5A (total 1.35×10¹¹ VG per animal), respectively, relative to the vehicle group (groups A6 and C1b). HTT protein KD was statistically significant (P<0.001 by one-way ANOVA with Tukey's multiple comparisons test) at the highest dose of 1.78×10¹³ vg/animal versus vehicle. The highest dose of 1.78×10¹³ VG per animal exhibited statistically greater HTT protein KD than was achieved with total doses of 1.35×10¹¹ and 1.35×10¹² VG per animal, respectively (P<0.01 for 1.78×10¹³ VG per animal vs 1.35×10¹¹ VG per animal, P<0.05 for 1.78×10¹³ VG per animal vs 1.35×10¹² VG per animal by one-way ANOVA with Tukey's multiple comparisons test). In addition, there was no statistically significant difference in HTT protein KD between the two lowest total doses (1.35×10¹¹ and 1.35×10¹² VG per animal) or between each of two lowest doses (1.35×10¹¹ and 1.35×10¹² VG per animal) and vehicle group tested.

HTT mRNA levels in the caudate were also measured by the bDNA assay, as described in Example 1, in the same caudate punches used for HTT protein quantitation, using the specific probe set against rhesus HTT. TBP, AARS and XPNPEP1. HTT mRNA levels were normalized to the geometric mean of the three housekeeping genes TBP, AARS and XPNPEP1, and then further normalized to the vehicle group. The average relative remaining caudate HTT mRNA and relative caudate HTT mRNA knockdown (KD), expressed as a percentage of vehicle (veh), as well as p-values generated by one-way ANOVA with Tukey's multiple comparisons, are presented in Table 104.

TABLE 104
HTT mRNA lowering in NHP caudate

| | | | Relative | | P value by | P value by | P value by |
|---|---|---|---|---|---|---|---|
| | | Total | remaining | | one-way | one-way | one-way |
| | Number of | dose | HTT mRNA | HTT mRNA KD | ANOVA | ANOVA | ANOVA |
| Group | animals | (VG) | (% of veh ± stdev) | (% of veh ± stdev) | (vs Group A5) | (vs Group A2) | (vs Group C4) |
| Group A6 | 3 | 0.0 | 100 ± 6 | 0 ± 6 | 0.9121 | 0.7377 | <0.0001 |
| Group C1b | 3 | 0.0 | | | | | |
| Group A5 | 3 | 1.35e11 | 95 ± 11 | 5 ± 11 | — | 0.9877 | 0.0002 |
| Group A2 | 3 | 1.35e12 | 93 ± 9 | 7 ± 9 | — | — | 0.0003 |
| Group C4 | 3 | 1.78e13 | 41 ± 16 | 59 ± 16 | — | — | — |

A dose-dependent HTT mRNA reduction was achieved in the caudate after bilateral intraputaminal and intrathalamic infusion of different total doses of VY-HTT01. Specifically, on average, there was 59%, 7% and 5% HTT mRNA KD for group C4 (total 1.78×10¹³ VG per animal), group A2 (total 1.35×10¹² VG per animal) and group A5 (total 1.35×10¹¹ VG per animal), respectively, relative to the vehicle group (groups A6 and C1b). Caudate HTT mRNA KD was statistically significant (P<0.001 by one-way ANOVA with Tukey's multiple comparisons test) at the highest dose of 1.78×10¹³ VG per animal versus vehicle. The highest dose of 1.78×10¹³ VG per animal exhibited statistically greater HTT mRNA KD than was achieved with total doses of 1.35×10¹¹ and 1.35×10¹² VG per animal, respectively (P<0.001 for 1.78×10¹³ VG per animal vs 1.35×10¹¹ VG per animal, and 1.78×10¹³ VG per animal vs 1.35×10¹² VG per animal by one-way ANOVA with Tukey's multiple comparisons test). In addition, there was no statistically significant difference in HTT mRNA KD between the two lowest total doses (1.35×10¹¹ and 1.35×10¹² VG per animal) or between each of two lowest doses (1.35×10¹¹ and 1.35×10¹² VG per animal) and vehicle group tested (P>0.05 by one-way ANOVA with Tukey's multiple comparisons test).

v. Thalamus HTT Protein and mRNA Levels

To evaluate HTT protein and mRNA lowering in the thalamus, 4 punches were collected from each animal (2 per hemisphere) for HTT and actin protein analysis by LC-MS/MS, resulting in a total of 12 punches per VY-HTT01 treatment group (3 animals per group), and 24 punches for two vehicle groups (3 animals for each vehicle group). Thalamus HTT protein levels were normalized to the levels of actin, and then further normalized to the vehicle group. The average relative remaining thalamus HTT protein and relative thalamus HTT protein knockdown (KD), expressed as a percentage of vehicle (veh), as well as p-values generated by one-way ANOVA with Tukey's multiple comparisons, are presented in Table 105.

TABLE 105
HTT protein lowering in NHP thalamus

| | | | Relative | | P value by | P value by | P value by |
|---|---|---|---|---|---|---|---|
| | | Total | remaining | | one-way | one-way | one-way |
| | Number of | dose | HTT protein | HTT protein KD | ANOVA | ANOVA | ANOVA |
| Group | animals | (VG) | (% of veh ± stdev) | (% of veh ± stdev) | (vs Group A5) | (vs Group A2) | (vs Group C4) |
| Group A6 | 3 | 0.0 | 100 ± 12 | 0 ± 12 | 0.2319 | 0.0694 | <0.0001 |
| Group C1b | 3 | 0.0 | | | | | |
| Group A5 | 3 | 1.35e11 | 82 ± 20 | 18 ± 20 | — | 0.9068 | 0.0009 |
| Group A2 | 3 | 1.35e12 | 74 ± 12 | 26 ± 20 | — | — | 0.0026 |
| Group C4 | 3 | 1.78e13 | 24 ± 3 | 76 ± 3 | — | — | — |

A significant and dose-dependent HTT protein reduction was achieved in the thalamus after bilateral combined intraputaminal and intrathalamic infusion of different total doses of VY-HTT01. Specifically, on average, there was 76%, 26% and 18% HTT protein KD for group C4 (1.78×10¹³ VG per animal), group A2 (1.35×10¹² VG per animal) and group A5 (1.35×10¹¹ VG per animal), respectively, relative to vehicle (groups A6 and C1b). The HTT protein KD in the thalamus was statistically significant (P<0.0001 by one-way ANOVA with Tukey's multiple comparisons test) at the highest dose of 1.78×10¹³ valanimal versus vehicle. The highest dose of 1.78×10¹³ VG per animal exhibited statistically greater HTT protein KD than was achieved with total doses of 1.35×10¹¹ and 1.35×10¹² VG per animal, respectively (P<0.001 for 1.78×10¹³ VG per animal vs 1.35×10¹¹ VG per animal. P<0.01 for 1.78×10¹³ VG per animal vs 1.35×10¹² VG per animal by one-way ANOVA with Tukey's multiple comparisons test). In addition, there was no statistically significant difference in HTT protein KD between the two lowest total doses (1.35×10¹¹ and 1.35×10¹² VG per animal) or between each of two lowest doses (1.35×10¹¹ and 1.35×10¹² VG per animal) and vehicle group tested.

HTT mRNA levels in the thalamus were also measured by the bDNA assay, as described in Example 1, in the same thalamus punches used for HTT protein quantitation, using the specific probe set against rhesus HTT, TBP, AARS and XPNPEP1. HTT mRNA levels were normalized to the geometric mean of the three housekeeping genes TBP, AARS and XPNPEP1, and then further normalized to the vehicle group. The average relative remaining thalamus HTT mRNA and relative thalamus HTT mRNA knockdown (KD), expressed as a percentage of vehicle (veh), as well as p-values generated by one-way ANOVA with Tukey's multiple comparisons, are presented in Table 106.

TABLE 106
HTT mRNA lowering in NHP thalamus

| | | | Relative | | P value by | P value by | P value by |
|---|---|---|---|---|---|---|---|
| | | Total | remaining | | one-way | one-way | one-way |
| | Number of | dose | HTT mRNA | HTT mRNA KD | ANOVA | ANOVA | ANOVA |
| Group | animals | (VG) | (% of veh ± stdev) | (% of veh ± stdev) | (vs Group A5) | (vs Group A2) | (vs Group C4) |
| Group A6 | 3 | 0.0 | 100 ± 13 | 0 ± 13 | 0.0041 | 0.0002 | <0.0001 |
| Group C1b | 3 | 0.0 | | | | | |
| Group A5 | 3 | 1.35e11 | 59 ± 21 | 41 ± 21 | — | 0.3533 | 0.0611 |
| Group A2 | 3 | 1.35e12 | 41 ± 4 | 59 ± 4 | — | — | 0.6651 |
| Group C4 | 3 | 1.78e13 | 29 ± 6 | 71 ± 6 | — | — | — |

Significant thalamic HTT mRNA reduction was achieved in the thalamus after bilateral intraputaminal and intrathalamic infusion of different total doses of VY-HTT01. Specifically, on average. 71%, 59% and 41% HTT snRNA KD for group C4 (1.78×10¹³ VG per animal), group A2 (1.35×10¹² VG per animal) and group A5 (1.35×10¹¹ VG per animal), respectively, relative to vehicle (groups A6 and C1b). The HTT mRNA KD in the thalamus was statistically significant (P<0.01 by one-way ANOVA with Tukey's multiple comparisons test) at all doses of VY-HTT01 versus vehicle. There was a trend towards dose-dependence of HTT mRNA KD although there was no statistically significant difference in thalamus HTT mRNA KD between different doses of VY-HTT01 (P>0.05 by one-way ANOVA with Tukey's multiple comparisons test). Moreover, the levels of thalamus HTT mRNA measured corresponded well with HTT protein levels in the same thalamus samples in that higher doses of VY-HTT01 trended towards more HTT mRNA and protein KD than lower doses of VY-HTT01.

vi. Relationship between HTT Protein and mRNA Levels

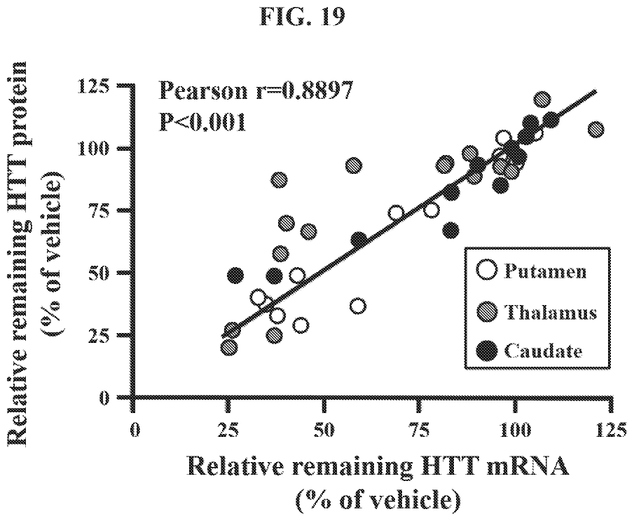

The relationship between relative HTT protein levels and relative HTT mRNA levels in all samples from putamen, caudate and thalamus from group C4 (1,78×10¹³ VG per animal), group A2 (1.35×10²¹ VG per animal), group A5 (1.35×10¹¹ VG per animal), and the vehicle group (groups A6 and C1b) was assessed using a Pearson correlation method. The graph showing the correlation of relative remaining NHP HTT protein and mRNA levels in the putamen, caudate, and thalamus is shown in FIG. 19.

Relative HTT protein levels as measured by the LC-MS/MS assay correlated well with relative HTT mRNA levels as measured by the bDNA assay (Pearson r=0.8897, P<0.001). For example, 59% remaining; HTT mRNA levels relative to vehicle corresponded to 50% remaining HTT protein levels relative to vehicle.

Together, the results for bilateral combined intraputaminal and intrathalamic administration of VY-HTT01 in the NHP demonstrated at 5 weeks after dosing: dose-dependent HTT protein lowering in NHP putamen, caudate and thalamus; dose-dependent HTT mRNA KD in the putamen and caudate, with a trend toward dose-dependent HTT mRNA KD in the thalamus; and, a positive correlation between relative remaining NHP HTT protein and mRNA levels in the putamen, caudate and thalamus.

### Example 8

### In Vivo Study of VY-HTT01 Dose Response Pharmacolo in the YAC128 Mouse Model of Huntington's Disease.

i. Study Design

The primary objective of this study was to identify dose levels of an AAV1. packaged AAV1-miRNA expression vector resulting in dose-dependent lowering of human mutant HTT mRNA and protein after intrastriatal infusion in the YAC128 mouse model of Huntington's disease, which express human full-length mutant huntingtin (HTT). Viral genome VYHT1 (SEQ ID NO: 1352) was packaged into AAV1 capsids to generate VY-HTT01 particles (may also be referred to as AAV1-VOYHT1) and formulated for delivery. in this study, only brain tissues at the infusion site (striatum) were evaluated for human mutant HTT mRNA and protein levels 4 weeks after dosing. The 4-week timepoint was selected based on earlier studies indicating that maximum human mutant HTT mRNA lowering is attained in the YAC128 mouse striatum at approximately this time following intrastriatal dosing of VY-HTT01.

This study involved thirty mus musculus FVB/YAC Tg(+) mice (YAC128; as described in Slow et al., Hum Mol Genet. 12: 1555-1567 (2003), and in Van Raamsdonk et al. Hum Mol Genet. 14(24):3823-35 (2005), the contents of each of which are incorporated herein by reference in their entirety), 2-3 months of age, randomly assigned into groups by even distribution by gender and age. On Day 1 all animals received bilateral injections of either vehicle (Group 1) or VY-HTT01 (Groups 2-5) at 0.5 μL/minute into the left and right striata via a stereotaxically positioned silica catheter connected to a syringe for 10 minutes. Four dose levels were evaluated: 8.8×10⁹, 2.8×10¹⁰, 8.8×10¹⁰, and 2.8×10¹¹vector genomes (vg or VG) per animal (total dose). The 2.8×10¹¹ VG total dose was the maximum feasible dose (MFD). Animal study groups, including detailed dosing parameters, are presented in Table 107.

TABLE 107
Study design

| | | | Vector Dose | | | |
|---|---|---|---|---|---|---|
| | | | Volume | Dosing | | Total |
| | Number of | | injection | titer | | dose |
| Group | animals | Description | (μL/side) | (VG/mL) | VG/side | (VG) |
| Group 1 | 6 | Vehicle | 5 | 0.0 | 0.0 | 0.0 |
| Group 2 | 6 | VY-HTT01, | 5 | 8.8e11 | 4.4e9 | 8.8e9 |
| | | low dose | | | | |
| Group 3 | 6 | VY-HTT01, | 5 | 2.8e12 | 1.4e10 | 2.8e10 |
| | | mid dose | | | | |
| Group 4 | 6 | VY-HTT01, | 5 | 8.8e12 | 4.4e10 | 8.8e10 |
| | | high dose | | | | |
| Group 5 | 6 | VY-HTT01, | 5 | 2.8e13 | 1.4e11 | 2.8e11 |
| | | MFD | | | | |

ii. Test Article Preparation and Administration

The test article used in the study contained VY-HTT01 formulated in aqueous solution containing 10 mM sodium phosphate, 1.5 mM potassium phosphate, 100 mM sodium chloride, 7% (w/v) sucrose, 0.001% (w/v) Poloxamer 188, pH 7.5. The vehicle control contained the formulation buffer only. VY-HTT01 was removed from −65° C. or below and allowed to thaw at room temperature. After thawing, VY-HTT01 was then pipetted up and down to mix and then placed on wet ice until the time of dilution. VY-HTT01 was diluted with formulation buffer prepared as detailed above. After completion of dilution, all dosing solutions were stored at 2-8° C. until administration. Dosing solutions were allowed to warm up to room temperature prior to injection. After the completion of dosing procedures to all study animals, the dose retains were maintained at 2-8° C. The retain vectors were titered by droplet digital PCR (ddPCR).

Prior to surgery, animals were anesthetized using 3% isoflurane and placed into a stereotaxic frame. Intracranial injections were performed as follows: five microliters (5 μL) of recombinant viral vectors or vehicle were injected into the striatum (AP, +0.50; ML, ±2.2; DV, −3.0 from bregma and dura; incisor bar. 0.0) using a 10 μL Hamilton syringe at the rate of 0.5 μL/min. The needle was left in place for 1 min following the completion of infusion, before it was raised out of the brain. One hour before surgery and for 24 h following surgery, the mice were administered sustained release Buprenorphine SR-LAB (1.0 mg/kg) subcutaneously for analgesia.

iii. Cage-Side Observations and Body Weights

Cage side observations were conducted prior to injection and performed daily starting after surgery. All mice were monitored for general health and signs of pain and/or illness daily. Cage-side observations showed no abnormalities for all animals in all dose groups during the study.

For body weights, mice were weighed immediately prior to test article administration and once weekly after dosing. Body weight measurements indicated that vehicle and VY-HTT01-injected animals had normal weight gain over time and showed no difference in body weight changes at 1 or 2 weeks after dosing (p>0.05 by two-way ANOVA with Tukey's multiple comparisons test). However, the MFD group had more body weight gain than the middle or high dose group at 3 and 4 weeks after dosing (p<0.01 by two-way ANOVA with Tukey's multiple comparisons test).

iv. Tissue Collection

Mice received an intraperitoneal injection of phenytoin (Euthasol®) (270 mg/kg) and then were perfused transcardially with cold 1×PBS, and brains removed. Brains were cut along the coronal axis using a mouse brain matrix and striatal regions were dissected from a 2 mm brain slab using a 3 mm biopsy punch. The overlying cortical region was also collected. Brain tissue was then flash-frozen in liquid nitrogen and stored at −80° C. for subsequent processing and analyses.

A 0.4 cm tail snip sample was collected from each mouse and stored at −60° C. or lower until confirmatory genotyping analysis. Genotyping analysis of mouse tail snip samples confirmed that all animals in the study were positive for the human mutant HTT gene.

v. HTT mRNA Quantification

Human HTT mRNA level and mouse X-Prolyl Aminopeptidase 1 (XPNPEP1) mRNA levels were determined by a quantitative (real time) reverse transcription polymerase chain reaction (RT-qPCR) TaqMan assay. Briefly, total RNA was extracted from striatal tissue samples using the mirVana™ miRNA Isolation Kit according to the manufacturer's protocol (QIAGEN). Complementary DNA synthesis was performed by reverse transcription using the QuantiTect® Reverse Transcription Kit (QIAGEN). All TaqMan assays and master mixes were ordered from Life Technologies and used according to the manufacturer's recommendations. qPCR was performed using the CFX384 real-time PCR system (BIO-RAD) and data were analyzed with the 2^{-ΔΔCt} method. HTT mRNA levels were normalized to XPNPEP1 mRNA levels, and then further normalized to the vehicle control group.

vi. HTT Protein Quantification

The Meso Scale Discovery (MSD) assay was performed according to the protocol described previously (Macdonald et al., PLoS One. May 9; 9(5):e96854 (2014), the contents of which are incorporated herein by reference in their entirety). Briefly, tissue sample homogenates were generated using the FastPrep system (MP Biomedicals) by lysing the tissue in MSD assay buffer (Tris lysis buffer—Meso Scale Discovery, supplemented with Phosphatase inhibitor II (Sigma), Phosphatase inhibitor III (Sigma), 2 mM PMSF, protease inhibitors (Complete, EDTA-free; Roche Diagnostics), and 10 mM NaF). Lysates were centrifuged for 20 min at 20,000 g at 4° C. Supernatant was collected, aliquoted, quickly frozen on dry ice, stored at −80° C., and used within 3-4 days to limit any possible detrimental effects of storage time on these samples. Total protein concentration was determined using a bicinchoninic acid assay (BCA, Thermo Scientific) according to standard procedures.

The MSD plate (MSD product #L15XA) was coated overnight at 4° C. with affinity purified mouse monoclonal antibody 2B7 raised against amino acids 1-17 of human HTT. The plate was washed 3 times with wash buffer and then blocked with blocking solution for 60 minutes at room temperature with shaking. The blocking solution was removed by inversion and then 25 μL of human HTT protein standards, QC samples (separately prepared human HTT protein standard) and diluted mouse brain samples were loaded by reverse pipetting. The plate was incubated for 60 minutes at room temperature with shaking. The plate was then washed 4 times with wash buffer and incubated with biotinylated mouse monoclonal antibody MW1 against the expanded polyglutamine domain of HTT protein. The plate was incubated for 60 minutes at room temperature with shaking. The plate was then washed 3 times with wash buffer and 25 μL of the Sulfo-tag streptavidin conjugate was added. The plate was incubated for 60 minutes at room temperature with shaking. The plate was then washed 3 times with wash buffer and 150 μL of read buffer was added. The plate was read on an MSD reader. Light intensity was then measured to quantify analytes in the sample. Concentrations of human HTT protein in the study samples were determined by computer interpolation from the calibration curve.

vii. Acceptance Criteria

For qPCR reactions, the average Ct values in no template controls were typically equal to or greater than 35. Therefore, samples with Ct values equal to or greater than 35 were defined as negative and excluded from data analyses. Any potential DNA contamination was eliminated with the genomic DNA Wipeout reagent in the QuantiTect® Reverse Transcription kit before cDNA synthesis and qPCR. Any cDNA samples were considered to have no tissue genomic DNA contamination if the Ct values for the corresponding RNA samples used as no RT control in the qPCR reaction were equal to or greater than 35.

For the MSD assay, the standard curve was required to contain at least six non-zero standards, after removal of rejected standards. The standard relative error (observed concentration-theoretical concentration)/ theoretical concentration ×100) was within ±25% for working standards. The relative error for quality control samples was within ±25%. The % CV between replicate concentrations was 25% or lower. At least 67% of the QC samples were required to meet the acceptance criteria, with 50% accepted QC samples at each level. The % CV between study sample well concentrations was 25% or lower.

viii. Dose-Dependent Mutant Human HTT mRNA Knockdown by VY-HTT01

To evaluate the dose-dependence of human mutant HTT mRNA lowering after intrastriatal dosing of VY-HTT01, human HTT and mouse XPNPEP1 mRNA levels in striatum samples were measured by RT-qPCR. Average Ct values and standard deviations (SDs) were calculated for both HTT and XPNPEP1, and ΔCt (CtHTT-CtXPNPEP1) was determined for each sample. The ΔΔCt (ΔCt of VY-HTT01-injected samples-average vehicle ΔCT) was then calculated, Relative HTT mRNA levels were calculated for each sample by the 2^{-ΔΔCt} method and expressed as the percentage relative to the vehicle control group. VY-HTT01 administration resulted in human mutant HTT mRNA knockdown (KD) in the striatum at 4 weeks after intrastriatal injection in YAC128 mice. The average relative remaining HTT mRNA and relative HTT mRNA knockdown (KD) in YAC128 mouse striatum after administration with VY-HTT01 at different dose levels, as well as p-values generated by statistical analyses, are presented in Table 108.

TABLE 108
HTT mRNA lowering in YAC128 striatum

| | | | Relative | | | | | |
|---|---|---|---|---|---|---|---|---|
| | | | remaining | | P value by | P value by | P value by | P value by |
| | | Total | HTT mRNA | HTT mRNA KD | one-way | one-way | one-way | one-way |
| | | dose | (% of veh; | (% of veh; | ANOVA | ANOVA | ANOVA | ANOVA |
| Group | Dose | (VG) | mean ± SD) | mean ± SD) | (vs Group 2) | (vs Group 3) | (vs Group 4) | (vs Group 5) |
| Group 1 | Veh | 0.0 | 100 ± 9 | 0 ± 9 | 0.0008 | <0.0001 | <0.0001 | <0.0001 |
| Group 2 | Low | 8.8e9 | 64 ± 18 | 36 ± 18 | — | 0.3827 | 0.0195 | 0.0023 |
| Group 3 | Mid | 2.8e10 | 49 ± 18 | 51 ± 18 | — | — | 0.5527 | 0.1469 |
| Group 4 | High | 8.8e10 | 37 ± 13 | 63 ± 13 | — | — | — | 0.9038 |
| Group 5 | MFD | 2.8e11 | 30 ± 5 | 70 ± 5 | — | — | — | — |

On average, 70±5% human HTT mRNA KD was observed at the maximal feasible dose (p<0.0001 relative to vehicle group), 63±13% human HTT mRNA KD at the high dose (p<0.0001 relative to vehicle group), 51±18% human HTT mRNA KD at the middle dose (p<0.0001 relative to vehicle group) and 36±18% human HTT mRNA KD at the low dose (p<0.001 relative to vehicle group). Different human HTT mRNA KD was seen between the low dose and high dose groups, and between the low dose and maximal feasible dose groups. These data demonstrate that VY-HTT01 suppressed striatal human HTT snRNA levels in a dose-dependent manner in the YAC128 mouse model of HD.

ix. Dose-Dependent Mutant Human HTT Protein Knockdown by YT-HTT01

To evaluate the dose-dependence of human mutant HTT protein lowering after intrastriatal dosing of VY-HTT01, the MSD assay was performed to quantitatively measure human mutant HTT protein in striatum tissue punches, VY-HTT01 administration resulted in human mutant HTT protein KD in the striatum at 4 weeks after intrastriatal injection in YAC128 mice.

The average relative remaining HTT protein and relative HTT protein knockdown (KD) in YAC128 mouse striatum after administration of VY-HTT01 at different dose levels, as well as p-values generated by statistical analyses, are presented in Table 109.

TABLE 109
HTT protein lowering in YAC128 striatum

| | | | Relative | HTT | P value by | P value by | P value by | P value by |
|---|---|---|---|---|---|---|---|---|
| | | Total | HTT protein | protein KD | one-way | one-way | one-way | one-way |
| | | dose | (% of veh; | (% of veh; | ANOVA | ANOVA | ANOVA | ANOVA |
| Group | Dose | (VG) | mean ± SD) | mean ± SD) | (vs Group 2) | (vs Group 3) | (vs Group 4) | (vs Group 5) |
| Group 1 | Veh | 0.0 | 100 ± 20 | 0 ± 20 | 0.0554 | 0.0282 | <0.0001 | <0.0001 |
| Group 2 | Low | 8.8e9 | 73 ± 21 | 27 ± 21 | — | 0.9980 | 0.0145 | 0.0038 |
| Group 3 | Mid | 2.8e10 | 70 ± 14 | 30 ± 14 | — | — | 0.0294 | 0.0079 |
| Group 4 | High | 8.8e10 | 41 ± 15 | 59 ± 15 | — | — | — | 0.9801 |
| Group 5 | MFD | 2.8e11 | 36 ± 7 | 64 ± 7 | — | — | — | — |

On average, 64±7% human HTT protein KD was observed at the maximal feasible dose (p<0.0001 relative to vehicle group), 59±15% human HTT protein KD at the high dose (p<0.0001 relative to vehicle group), 30±14% human HTT protein KD at the middle dose (p<0.05 relative to vehicle group) and 27±21% human HTT protein KD at the low dose (p<0.0554 relative to vehicle group). Different human HTT protein KD was seen between the maximal feasible dose and middle dose groups, maximal feasible dose and low dose group, high and middle dose groups, and high and low dose groups. These data demonstrate that VY-HTT01 suppressed human HTT protein levels in the striatum in a dose-dependent manner in the YAC128 mouse model of HD.

This study demonstrated that intrastriatal injection of VY-HTT01 resulted in striatal human mutant HTT mRNA and protein KD in a dose-dependent manner, ranging from 70% HTT mRNA KD at the maximal feasible dose to 36% HTT mRNA KD at the low dose, on average, and 64% human HTT protein KD at the maximal feasible dose to 27% HTT protein KD at the low dose, on average. No abnormal cage-side observations were detected for all animals in the study and comparable body weight gain was seen for all groups of animals. These data demonstrated that VY-HTT01 intrastriatal doses ranging from 8.8×10⁹ VG to 2.8×10¹¹ VG per animal suppressed human mutant HTT mRNA and protein expression in the striatum in the YAC128 mouse model of HD in a dose-dependent manner, with maximal human mutant HTT protein lowering attained at a dose of 8.8×10¹⁰ VG per animal.

### Example 9

### In Vivo Study of VY-HTT01 Efficacy in the YAC128 Mouse Model of Huntington's Disease

i. Study Design

The primary objective of this study was to investigate the efficacy of an AAV1 packaged viral genome VOYHT1 (SEQ ID NO: 1352; hereinafter together referred to as AAV1-VOYHT1 or VY-HTT01)) on motor function following bilateral intrastriatal infusion in the YAC128 mouse, a model of Huntington's disease expressing human full-length mutant huntingtin (HTT). Further, this study was designed to evaluate the relationship between efficacy of VY-HTT01 administration on motor function and VY-HTT01 dose, as well as HTT mRNA and protein lowering. Immunohistochemical analyses using various cellular and biochemical markers were performed, providing insight into nervous system tissue integrity. Cage-side observations, and body and brain weight data, were also recorded.

On the basis of data obtained from dose response pharmacology experiments described in Example 8 (above), and in an effort to provide maximal and graded reduction of human mutant HTT protein, three dose levels of VY-HTT01 were selected for evaluation in the present study, as follows: 8.8×10¹⁰ VG/animal (high dose), 2.8×10¹⁰ VG/animal (middle or mid dose), and 8.8×10⁹ VG/animal (low dose). Briefly, data from Example 8 demonstrated 59%, 30% and 27% human mutant HTT protein lowering, respectively, at these dose levels at 4 weeks after bilateral intrastriatal administration of VY-HTT01. In this study, brain tissues at the infusion site (striatum) were evaluated for human mutant HTT mRNA and protein levels at 16 weeks and at 24 weeks after administration (post-dosing).

This study involved fifty-two (52) male and forty-four (44) female mils musculus FVB/YAC Ts(+) mice (YAC128; as described in Slow et al., Hum Mol Genet.12:1555-1567 (2003), and in Van Raamsdonk et al. Hum Mol Genet. 14(24):3823-35 (2005), the contents of each of which are incorporated herein by reference in their entirety), as well as thirteen (13) male and eleven (11) female C57BL/6 (WT) mice (2-3 months old), randomly assigned into groups by even distribution of gender, age, and body weight, and results from baseline behavioral tests. On Day 1, animals received bilateral injections of 5 μL each of either vehicle (Groups 1 and 2) or VY-HTT01 (Groups 3-5) at 0.5 μL/minute over 10 minutes into the left and right striata via a stereotaxically positioned silica catheter connected to a syringe. The first cohort of mice comprising six (6) males and six (6) females in each of Groups 1-5 for this study were euthanized 24 weeks post-dosing and used for histological evaluation, as well as in-life observations (e.g., cage-side and body weight) and behavioral testing. The second cohort of mice comprising 7 males and 5 females in each of Groups 1-5 for this study were euthanized 16 weeks post-dosing and used for in-life observations, behavioral testing and pharmacological evaluation. The details for groups, animal genotype, test articles and dosing paradigm are presented in Table 110.

TABLE 110
Study design

| | | | | Vector Dose | | | | |
|---|---|---|---|---|---|---|---|---|
| | | | | Volume | Dosing | | Total | |
| | | Number of | | injection | titer | | dose | In-life |
| Group | Genotype | animals | Description | (μL/side) | (VG/mL) | VG/side | (VG) | (Weeks) |
| Group 1 | WT | 24 | Vehicle | 5 | 0.0 | 0.0 | 0.0 | 16, 24 |
| Group 2 | YAC128 | 24 | Vehicle | 5 | 0.0 | 0.0 | 0.0 | 16, 24 |
| Group 3 | YAC128 | 24 | VY-HTT01, | 5 | 8.8e11 | 4.4e9 | 8.8e9 | 16, 24 |
| | | | low dose | | | | | |
| Group 4 | YAC128 | 24 | VY-HTT01, | 5 | 2.8e12 | 1.4e10 | 2.8e10 | 16, 24 |
| | | | mid dose | | | | | |
| Group 5 | YAC128 | 24 | VY-HTT01, | 5 | 8.8e12 | 4.4e10 | 8.8e10 | 16, 24 |
| | | | high dose | | | | | |

ii. Test Article Preparation and Administration

The test article used in the study contained VY-HTT01 formulated in aqueous solution containing 10 mM sodium phosphate, 1.5 mM potassium phosphate, 100 mM sodium chloride, 7% (w/v) sucrose. 0.001% (w/v) Poloxamer 188, pH 7.5. The vehicle control contained the formulation buffer only. VY-HTT01 was removed from -65° C. or below and allowed to thaw at room temperature. After thawing, VY-HTT01 was then pipetted up and down to mix and then placed on wet ice until the time of dilution. VY-HTT01 was diluted with formulation buffer prepared as detailed above. After completion of dilution, all dosing solutions were stored at 2-8° C. until administration. Dosing solutions were allowed to warm up to room temperature prior to injection. After the completion of dosing procedures to all study animals, the dose retains were maintained at 2-8° C. The retain vectors were titered by droplet digital PCR (ddPCR).

Animals were anesthetized by injection with ketamine (10 mg/mL), xylazine (1 mg/mL) and acepromazine (0.3 mg/mL) solution (160-200 μL/20 g) and placed into a stereotaxic frame. Intracranial injections were performed as follows: five microliters (5 μL) of recombinant viral vectors or vehicle were injected into the striatum (AP, +0.5; ML, ±2.0, DV, −3.8) from bregma and dura; incisor bar, 0.0) using a 10 μL Hamilton syringe at the rate of 0.5 μL/min. The needle was left in place for 3 minutes following the completion of infusion, before it was raised out of the brain. Acetaminophen was provided in the drinking water 3 days before surgery and 3 days after surgery as pre- and post-operative analgesic.

iii. Cage-Side Observations, Body Weight, and Brain Weight

Cage-side observations were conducted prior to injection and performed daily starting after surgery. All mice were monitored for general health and signs of pain and/or illness daily. Cage-side observations showed no abnormalities for all animals in all dose groups during the study, except one female WT mouse found dead at 13 weeks post injection.

For body weights, mice were weighed immediately prior to test article administration and weekly after dosing. Brain weights were recorded after removal from the animal. Body weight measurements from 0- to 16-weeks post dosing indicated that, in general, YAC128 mice gained more weight over time than their WT littermates. However, there were no differences in body weight changes between YAC128 groups administered vehicle or any doses of VY-HTTO by one-way ANOVA with Tukey's post hoc test (p>0.05). At 16 weeks post dosing, 11-12 mice in each group were euthanized for pharmacological analysis. The weekly body weight measurements were continued on the remaining mice, which showed similar changes of body weight and body weight gain across groups. Brain weight measurements also showed no differences between groups at 16- or 24-weeks post-dosing by one-way ANOVA with Tukey's post hoc test.

iv. In-Life Behavioral Test

The accelerating rotarod test was performed to assess the effects of VY-HTT01 on mouse motor function and motor coordination. YAC128 mice were trained on the rotarod for two consecutive days (day 1 and 2) and tested on day 3. On the first training day, the rotarod was set to a constant speed of 5 RPM for 300 seconds. Mice that fell off the rod before completion of the 300-second time period were placed back on the rod until the full 300-second period expired. On the second day of training, the rotarod was set to accelerate from 5 to 40 rpm over 300 seconds. Mice that fell off the rod were placed back on the rod until it completed the 300-second training period. On day 3 (test day), the mice were placed on the rotarod set to accelerate from 5 to 40 rpm over 300 seconds. The latency to fall, defined by the time elapsed until the animal falls from the rotarod, was recorded over three trials. The rotarod test was carried out at pre-dosing for baseline measurement and was repeated every 4 weeks after until necropsy. All animals went through the rotarod tests performed at pre-dosing, and 4-, 8-, 12- and 16-weeks post-doing (n=23-24 per group), and a subset of them continued the rotarod test at 20- and 24-weeks post-doing (n=12 per group).

v. Tissue Collection

At sixteen weeks post VY-HTT01 administration, a subset of 11-12 animals from each group (6-7 males and 5 females) received an intraperitoneal injection of ketamine (10 mg/mL), xylazine (1 mg/mL) and acepromazine (0.3 mg/mL) solution (160˜200 μL/20 g), Once under anesthesia, mice were weighed and body weights were recorded. The toe pinch-response method was used to determine depth of anesthesia. Once unresponsive, mice were perfused transcardially with cold 1×PBS, and brains were carefully removed. Brain weights were recorded and then brains were cut along the coronal axis using a mouse brain matrix, Tissue samples were collected from the left and right striatum of a 2 mm-thick brain slab using a 3 mm biopsy punch. The overlying cortical region was also collected for potential biochemical analysis. These striatal and cortical samples were then flash-frozen in liquid nitrogen and stored at −80° C. for processing and mRNA or protein analyses.

The remaining 12 animals (6 males and 6 females) from each group were euthanized at 24 weeks after dosing. Striatum tissue punches from 6 mice (3 males and 3 females) in each group were collected as above (dissected and snap-frozen) for HTT mRNA measurement by qRT-PCR and HTT protein measurement by MSD. The brains from the remaining 6 animals (3 males and 3 females) per group were dissected and post-fixed with 4% paraformaldehyde (PFA) after perfusion with 1×PBS and 4% PFA for immunohistochemical analyses.

A 0.4 cm sample of tail from each mouse was collected and stored at −60° C. or lower until confirmatory genotyping analysis. Genotyping analysis of mouse tail snip samples confirmed that all 96 YAC128 animals in the study were positive and all 24 WT mice were negative for the human mutant HTT gene, as expected.

vi. HTT mRNA Quantification

Human HTT mRNA levels were determined in accordance with procedures described in Example 8 (above).

vii. HTT Protein Quantification

Human HTT protein levels were determined in accordance with procedures described in Example 8 (above).

viii. Immunohistochemical Analyses

Immunohistochemistry (IHC) staining for HTT aggregates (EM48), neurodegeneration (Fluoro-Jade C). striatal medium spiny neurons (DARPP32), neuronal (NeuN) and glial-specific markers (GFAP for astrocytes and Iba1 for microglia), were conducted on mouse brains using standard protocols.

ix. Acceptance Criteria

Acceptance criteria for qPCR reactions and for the MSD assay used in this study were identical to those described in Example 8 (above).

x. Effects of VY-HTT01 on YAC128 Mouse Motor Function and Motor Coordination

The rotarod test was performed at pre-dosing and repeated every 4 weeks post-dosing, up to 16 weeks post-dosing (n=23-24 mice per group). Thereafter, the rotarod test was continued with a subset of mice (n=12 mice per group) at 20- and 24-weeks post-dosing. The latency to fall of each animal was recorded and percent (%) change of latency to fall (post-dosing latencylpre-doing latency×100%) was calculated. YAC128 mice had impaired motor function compared with WT littermates. Based on the absolute latency to fall, YAC128 mice showed a shorter latency to fall than WT littermates at baseline (p<0.0001, two-way ANOVA with Dunnett's post hoc test). At 16 weeks post-dosing, VY-HTT01 high and low dose groups improved the absolute latency to fall in YAC128 mice (p=0.025 and 0.034, respectively, versus vehicle), while the VY-HTT01 middle dose group showed a trend (p=0.052 versus vehicle) towards improved latency to fall. Rotarod test absolute latency to fall (seconds) data are presented in Table 111.

TABLE 111
Latency to fall (seconds) in the rotarod test

| | | Pre-dose | 4-week | 8-week | 12-week | 16-week |
|---|---|---|---|---|---|---|
| | | baseline | post-dose | post-dose | post-dose | post-dose |
| | | (mean ± SD, | (mean ± SD, | (mean ± SD, | (mean ± SD, | (mean ± SD, |
| Genotype | Description | n = 24/group) | n = 24/group) | n = 24/group) | n = 24/group | n = 23-24/group |
| WT | Vehicle | 264 ± 59 | 224 ± 65 | 247 ± 47 | 239 ± 61 | 239 ± 62 |
| YAC128 | Vehicle | 124 ± 80 | 138 ± 55 | 127 ± 44 | 108 ± 44 | 113 ± 38 |
| YAC128 | VY-HTT01, | 121 ± 72 | 133 ± 60 | 157 ± 49 | 147 ± 55 | 155 ± 64 |
| | high dose | | | | | |
| YAC128 | VY-HTT01, | 120 ± 86 | 133 ± 70 | 141 ± 84 | 134 ± 73 | 154 ± 72 |
| | mid dose | | | | | |
| YAC128 | VY-HTT01, | 122 ± 67 | 118 ± 63 | 125 ± 63 | 130 ± 62 | 151 ± 61 |
| | low dose | | | | | |

As assessed by the percent change in the latency to fall, VY-HTT01 high dose (8.8×10¹³ VG per animal) increased the latency to fall by ˜21% at 12 weeks (p=0.023) and by ˜27% at 16 weeks (p=0.021) post-dosing versus vehicle (one-way ANOVA with Dunnett's post hoc test), indicating a rescue of motor deficits. VY-HTT01 middle- and low-dose groups also showed increases in the latency to fall by 28% (p=0.037) and 24% (p=0.033), respectively, at 16 weeks post-dosing versus vehicle (one-way ANOVA with Dunnett's post hoc test). Rotarod test percent (%) latency to fall data are presented in Table 112.

TABLE 112
Percent (%) change in latency to fall in the rotarod test

| | | Pre-dose | 4-week | 8-week | 12-week | 16-week |
|---|---|---|---|---|---|---|
| | | baseline | post-dose | post-dose | post-dose | post-dose |
| | | (mean ± SD, | (mean ± SD, | (mean ± SD, | (mean ± SD, | (mean ± SD, |
| Genotype | Description | n = 24/group) | n = 24/group) | n = 24/group) | n = 24/group | n = 23-24/group |
| WT | Vehicle | 100 ± 22 | 85 ± 25 | 94 ± 18 | 90 ± 23 | 91 ± 23 |
| YAC128 | Vehicle | 100 ± 65 | 112 ± 44 | 103 ± 36 | 87 ± 35 | 91 ± 31 |
| YAC128 | VY-HTT01, | 100 ± 59 | 110 ± 49 | 129 ± 41 | 121 ± 46 | 127 ± 53 |
| | high dose | | | | | |
| YAC128 | VY-HTT01, | 100 ± 72 | 111 ± 58 | 118 ± 70 | 112 ± 61 | 128 ± 60 |
| | mid dose | | | | | |
| YAC128 | VY-HTT01, | 100 ± 55 | 96 ± 51 | 102 ± 52 | 106 ± 50 | 124 ± 50 |
| | low dose | | | | | |

These results suggest that motor function was improved at 16 weeks after administration with all three doses of VY-HTT01, and that there was faster onset of motor improvement in the high dose group. In addition, the rotarod test was continued with a subset of mice (12 mice per group) at 20- and 24-weeks post-dosing, and VY-HTT01 efficacy was maintained.

xi. Dose-Dependent Human Mutant HU mRATA Knockdown by VY-HTT01

After completion of behavioral assessment as described above, the dose-dependence of human HTT mRNA lowering was assessed in striatum samples from the left hemisphere of 11-12 animals or 6 animals per group at 16 or 24 weeks, respectively, following intrastriatal dosing of VY-HTT01. Human HTT and mouse XPNPEP1 mRNA (as an internal control) levels were measured by RI qPCR. All samples were run in duplicate for qPCR. Average Ct values and standard deviations (SD) were calculated for both HTT and XPNPEP, and ΔCt (CtHTT-CtXPNPEP1) was determined for each sample. The ΔΔCt (ΔCt of VY-HTT01-injected samples-average vehicle ΔCT) was then calculated. Relative human HTT mRNA levels were calculated for each sample by the 2 ΔΔCt method and expressed as the percentage relative to the vehicle control group. VY-HTT01. administration resulted in human HTT mRNA KD in the striatum at 16 and 24 weeks after intrastriatal injection in YAC128 mice.

At 16 weeks post-dosing, 48±19% human HTT mRNA KD was observed at the high dose (p<0.0001 relative to vehicle group), 57±24% human HTT mRNA KD at the middle dose (p<0.0001 relative to vehicle group) and 64±6% human HTT mRNA KD at the low dose (p<0.0001 relative to vehicle group). There was no difference in HTT mRNA levels among high, middle and low dose groups at 16 weeks post-dosing. Human HTT mRNA levels in YAC128 mouse striatum at 16 weeks after VY-HTT01. administration at different dose levels, as well as p-values generated by statistical analyses, are presented in Table 113.

TABLE 113
HTT mRNA lowering in YAC128 striatum at 16 weeks post dose

| | | 16 weeks post dose | | | | | |
|---|---|---|---|---|---|---|---|
| | | (% of Veh; mean ± SD, | | | | | |
| | | n = 11-12/group) | | P value by | P value by | P value by | P value by |
| | | Relative | | one-way | one-way | one-way | one-way |
| | | remaining | HTT | ANOVA | ANOVA | ANOVA | ANOVA |
| Genotype | Dose | HTT mRNA | mRNA KD | (vs Veh) | (vs High) | (vs Mid | (vs Low) |
| YAC128 | Vehicle | 100 ± 12 | 0 ± 12 | — | <0.0001 | <0.0001 | <0.0001 |
| YAC128 | VY-HTT01, | 52 ± 19 | 48 ± 19 | — | — | 0.599 | 0.113 |
| | high dose | | | | | | |
| YAC128 | VY-HTI01, | 43 ± 24 | 57 ± 24 | — | — | — | 0.719 |
| | mid dose | | | | | | |
| YAC128 | VY-HTT01, | 36 ± 6 | 64 ±6 | — | — | — | — |
| | low dose | | | | | | |

At 24 weeks post-dosing, VY-HTT01 high-, middle- and low-dose administration resulted in human HTT mRNA KD by 67±4%, 61±4%, and 53±11%, respectively, in YAC128 mouse striatum, relative to the vehicle group. There was no difference in HTT mRNA levels among high, middle and low dose groups at 24 weeks post-dosing. Human HTT mRNA levels in YAC128 mouse striatum at 24 weeks after VY-HTT01 administration at different dose levels, as well as p-values generated by statistical analyses, are presented in Table 114.

TABLE 114
HTT mRNA lowering in YAC128 striatum at 24 weeks post dose

| | | 24 weeks post dose | | | | | |
|---|---|---|---|---|---|---|---|
| | | (% of Veh; mean ± SD, | | | | | |
| | | n = 6/group) | | P value by | P value by | P value by | P value by |
| | | Relative | | one-way | one-way | one-way | one-way |
| | | remaining | HTT | ANOVA | ANOVA | ANOVA | ANOVA |
| Genotype | Dose | HTT mRNA | mRNA KD | (vs Veh) | (vs High) | (vs Mid | (vs Low) |
| YAC128 | Vehicle | 100 ± 17 | 0 ± 17 | — | <0.0001 | <0.0001 | <0.0001 |
| YAC128 | VY-HTT01, | 33 ± 4 | 67 ± 4 | — | — | 0.749 | 0.104 |
| | high dose | | | | | | |
| YAC128 | VY-HTT01, | 39 ± 4 | 61 ± 4 | — | — | — | 0.502 |
| | mid dose | | | | | | |
| YAC128 | VY-HTT01, | 47 ± 11 | 53 ± 11 | — | — | — | — |
| | low dose | | | | | | |

Overall, intrastriatal administration of VY-HTT01 at 8.8×10⁹ to 8.8×10¹⁰ vg per animal resulted in similar 48-64% striatal human HTT mRNA suppression at 16 weeks post-dosing, and 53-67% striatal human HTT mRNA suppression at 24 weeks post-dosing in YAC128 mice.

xii. Dose-Dependent Human Mutant HTT Protein Knockdown by VY-HTT01

After completion of behavioral assessments as described above, human mutant HTT protein lowering was assessed in the striatum samples from the right hemisphere of 11-12 animals or 6 animals per group at 16 weeks or 24 weeks, respectively, following intrastriatal dosing of VY-HTT01. VY-HTT01 injection resulted in human mutant HTT protein KD in the striatum. On average, human mutant HTT protein levels were suppressed by 65±10% at the high dose (p<0.0001), 52±13% at the middle dose (p<0.0001) and 38±13% at the low dose (p<0.0001), at 16-week post-dosing, wherein the significance was assessed using a one way ANOVA with Tukey post-hoc test. This human mutant HTT protein lowering was dose dependent, with differences between the high versus middle dose (p<0.05), high versus low dose (p<0.0001) and middle versus low dose (p<0.05) groups. Human HTT protein levels in YAC128 mouse striatum at 16 weeks after VY-HTT01 administration at different dose levels, as well as p-values generated by statistical analyses, are presented in Table 115.

TABLE 115
HTT protein lowering in YAC128 striatnm at 16 weeks post dose

| | | 16 weeks post dose | | | | | |
|---|---|---|---|---|---|---|---|
| | | (% of Veh; mean ± SD), | | | | | |
| | | n = 11-12/group) | | P value by | P value by | P value by | P value by |
| | | Relative | | one-way | one-way | one-way | one-way |
| | | remaining | HTT | ANOVA | ANOVA | ANOVA | ANOVA |
| Genotype | Dose | HTT protein | protein KD | (vs Veh) | (vs High) | (vs Mid | (vs Low) |
| YAC128 | Vehicle | 100 ± 11 | 0 ± 11 | — | <0.0001 | <0.0001 | <0.0001 |
| YAC128 | VY-HTT01, | 35 ± 10 | 65 ± 10 | — | — | 0.043 | <0.0001 |
| | high dose | | | | | | |
| YAC128 | VY-HTT01, | 48 ± 13 | 52 ± 13 | — | — | — | 0.025 |
| | mid dose | | | | | | |
| YAC128 | VY-HTT01, | 62 ± 13 | 38 ± 13 | — | — | — | — |
| | low dose | | | | | | |

At 24 weeks post-dosing, human mutant HTT protein levels were suppressed by 71±8% at the high dose (p<0.0001), 50±15% at the middle dose (p<0.0001) and 47±9% at the low dose (p<0.0001), wherein the significance was assessed using one-way ANOVA with Tukey post-hoc test. The human HTT protein lowering at 24 weeks post-dosing was also dose-dependent, with differences between the high-versus middle-dose (p<0.05) and high versus low dose (p<0.01), but not between the middle- and low-dose groups. Human HTT protein levels in YAC128 mouse striatum at 24 weeks after VY-HTT01 administration at different dose levels, as well as p-values generated by statistical analyses, are presented in Table 116.

TABLE 116
HTT protein lowering in YAC128 striatnm at 24 weeks post dose

| | | 24 weeks post dose | | | | | |
|---|---|---|---|---|---|---|---|
| | | (% of Veh; mean ± SD, | | | | | |
| | | n = 6/group) | | P value by | P value by | P value by | P value by |
| | | Relative | | one-way | one-way | one-way | one-way |
| | | remaining | HTT | ANOVA | ANOVA | ANOVA | ANOVA |
| Genotype | Dose | HTT protein | protein KD | (vs Veh) | (vs High) | (vs Mid | (vs Low) |
| YAC128 | Vehicle | 100 ± 13 | 0 ± 13 | — | <0.0001 | <0.0001 | <0.0001 |
| YAC128 | VY-HTT01, | 29 ± 8 | 71 ± 8 | — | — | 0.027 | 0.009 |
| | high dose | | | | | | |
| YAC128 | VY-HTT01, | 50 ± 15 | 50 ± 15 | — | — | — | 0.962 |
| | mid dose | | | | | | |
| YAC128 | VY-HTT01, | 53 ± 9 | 47 ± 9 | — | — | — | — |
| | low dose | | | | | | |

Together, these results demonstrate that intrastriatal dosing of VY-HTT01 lowers human mutant HTT protein expression in the striatum in a similar dose-dependent manner in the YAC128 mouse model of HD at 16- and 24-weeks post-dosing.

xiii. Immunohistochemical Analyses of HTT Aggregates and Cellular Markers Following Intrastriatal Dosing with VY-HTT01

At 24 weeks following intrastriatal dosing with VY-HTT01, the striatum from 6 animals per group were evaluated by immunohistochemical (IHC) analyses of markers for HTT aggregates (EM48), neuronal nuclear antigen (NeuN), neuronal degeneration (Fluoro-Jade C), astrocytes (glial fibrillary acidic protein; GFAP), microglia (ionized calcium binding adaptor molecule 1; Iba1) and medium-size spiny neurons (DARPP32).

No differences in immunoreactivity were observed across vehicle- and VY-HTT01-injected YAC128 mouse groups for any marker. IHC using EM48 showed diffuse nuclear staining in the striatum of YAC128 mice; however, nuclear aggregates were not observed, which is consistent with the published literature (Slow et al., Proc. Natl Acad. Sci. USA 102:11402-11407 (2005), the contents of which are incorporated herein by reference in their entirety). The density of NeuN-positive cells in the striatum was similar across all groups, showing no change in NeuN-positive neuron density. Neuronal degeneration was not identified in any group, based on Fluoro-Jade C immunoreactivity. No changes in Iba1-positive microglial density or GFAP positive astrocyte proliferations were identified. Medium-size spiny neurons were detected in all groups, with no significant differences in immunoreactivity, based on DARPP32 staining.

In this study, no treatment-related differences in neuronal cell density, gliosis, or body weight were identified in YAC128 mice.

This study demonstrated that intrastriatal injection of VY-HTT01 (8.8×10⁹ to 8.8×10¹⁰ VG per animal) resulted in striatal human HTT mRNA lowering ranging from 48% to 64%, accompanied by dose-dependent striatal human mutant HTT protein lowering by 38-65% at 16 weeks post-dosing in YAC128 mice. In addition, intrastriatal injection of VY-HTT01 (8.8×10⁹ to 8.8×10¹⁰ VG per animal) resulted in striatal human HTT mRNA lowering ranging from 53% to 67%. accompanied by dose-dependent striatal human mutant HTT protein lowering by 47-71% at 24 weeks post-dosing in YAC128 mice. VY-HTT01 at all three doses ameliorated motor coordination deficits in the rotarod test at 16 weeks post-dosing in YAC128 mice, as well as at 12 weeks post-dosing with VY-HTT01 at the high dose. The high dose (8.8×10¹⁰ VG per animal) of VY-HTT01 resulted in human mutant HTT protein lowering by 65% and 71% in striatum at 16- and 24-weeks post-dosing. The middle dose (2.8×10¹⁰ VG per animal) of VY-HTT01 reduced human mutant HTT protein by 52% and 50% in striatum at 16- and 24-weeks post-dosing in YAC128 mice, corresponding to improvement of motor function at 16 weeks post-dosing. The low dose (8.8×10⁹ VG per animal) of VY-HTT01 resulted in human mutant HTT protein reduction by 38% and 47% in the striatum at 16- and 24-weeks post-dosing in YAC128 mice, and improved motor function at 16 weeks post-dosing. No abnormal cage-side observations were found in any animals in the study. Comparable body weight gain was seen for all groups over 16 weeks, and there were no differences in brain weight at 16- or 24-weeks post-dosing. Together, these data demonstrate that: (1) intrastriatal infusion of VY-HTT01 lowers human HTT mRNA and human mutant HTT protein expression in the YAC128 mouse striatum in a dose-dependent manner; (2) commensurate with human mutant HTT protein lowering, VY-HTT01 improves motor function in YAC128 mice; and (3) VY-HTT01 injection was not associated with any changes in general health of the animals or neuronal injury.

### Example 10

### In Vivo Study of VY-HTT01 Dose Response Pharmacology in the BACHD Mouse Model of Huntington's Disease

i. Study Design

The primary objective of this study was to identify dose levels of an AAV1 packaged viral genome, VOYHT1 (SEQ ID NO: 1352), (hereinafter referred to as AAV1-VOYHT1 or VY-HTT01) resulting in dose-dependent lowering of human mutant HTT mRNA and protein after intrastriatal infusion in the BACHD mouse model of Huntington's disease, which expresses human full-length mutant huntingtin (HTT). In this study, only brain tissues at the infusion site (striatum) were evaluated for human mutant HTT mRNA and protein levels 4 weeks after dosing. The 4-week timepoint was selected based on earlier studies indicating that maximum human mutant HTT mRNA lowering is attained in the YAC128 mouse striatum at approximately this time following intrastriatal dosing of VY-HTT01. Experimental procedures were similar to that described in Example 8.

This study involved forty-five (45) female inns museums C57BL/6-Tg(HTT*97Q)IXwy/J (BACHD) mice, 2-3 months of age, randomly assigned into groups by even distribution of age and body weight. On Day 1 all animals received bilateral injections of either vehicle or VY-HTT01 at 0.5 μL/minute into the left and right striata via a stereotaxically positioned silica catheter connected to a syringe. The maximum feasible does (MFD) was determined by the maximum achievable vector titer and the maximum volume (5 μL/side) for intrastriatal injection in the mouse. Animal study groups, including detailed dosing parameters, are presented in Table 117.

TABLE 117
Study design

| | | | Vector Dose | | | |
|---|---|---|---|---|---|---|
| | | | Volume | | | Total |
| | Number of | | injection | Dosing titer | | dose |
| Group | animals | Dose | (μL/side) | (VG/mL) | VG/side | (VG) |
| Group 1 | 9 | Vehicle | 5 | 0.0 | 0.0 | 0.0 |
| Group 2 | 9 | VY-HTT01, | 5 | 8.8e11 | 4.4e9 | 8.8e9 |
| | | low dose | | | | |
| Group 3 | 9 | VY-HTT01, | 5 | 2.8e12 | 1.4e10 | 2.8e10 |
| | | mid dose | | | | |
| Group 4 | 9 | VY-HTTOL | 5 | 8.8e12 | 4.4e10 | 8.8e10 |
| | | high dose | | | | |
| Group 5 | 9 | VY-HTT01, | 5 | 2.8e13 | 1.4e11 | 2.8e11 |
| | | MFD | | | | |

ii. Test Article Preparation and Administration

The test article preparation procedure was similar to that described in Example 8.

Prior to surgery, animals were anesthetized by injection with ketamine (10 mg/mL), xylazine (1 mg/mL) and acepromazine (0.3 mg/mL) solution (160-200 μL/20 g) and placed into a stereotaxic frame. Intracranial injections were performed as follows: five microliters (5 μL) of recombinant viral vectors or vehicle were injected into the striatum (A/P +0.5, M/L ±2.0, DV −3.8 from bregma and dura; incisor bar, 0.0) using a 10 μL Hamilton syringe at the rate of 0.5 μL/min. The needle was left in place for 3 minutes following the completion of infusion, before it was raised out of the brain. Acetaminophen was provided in the drinking water 3 days before surgery and 3 days after surgery as pre- and post-operative analgesic.

iii. Cage-Side Observations, Body Weight, and Brain Weight

Cage-side observations were conducted prior to injection and performed daily starting after surgery. All mice were monitored daily for general health and signs of pain and/or illness. Cage-side observations showed no abnormalities for all animals in all dose groups during the study.

For body weights, mice were weighed immediately prior to test article administration and weekly after dosing. Brain weights were recorded after removal from the animal. Body weight measurements indicated that animals injected with vehicle or VY-HTT01 had normal weight gain over time and showed no difference in body weight changes. Brain weight measurement showed no differences across vehicle and VY-HTT01 groups.

iv. Tissue Collection

Mice received an intraperitoneal injection of ketamine (10 mg/mL), xylazine (1 mg/mL) and acepromazine (0.3 mg/mL) solution (160-200 μL/20 g). Once under anesthesia, mice were weighed and body weights were recorded. The toe pinch-response method was used to determine depth of anesthesia. Once unresponsive, mice were perfused transcardially with cold 1×PBS, and brains removed carefully and brain weight was recorded. Brains were cut along the coronal axis using a mouse brain matrix and striatal regions were dissected from a 2 mm-thick brain slab using a 3 mm biopsy punch. The overlying cortical region was also collected, Brain tissue was then flash-frozen in liquid nitrogen and stored at -80° C. for processing and analyses.

Mouse tail snips were used for DNA purification and genotyping analysis. In brief, for genotyping, the reaction mixture contained 900 mM of forward and reverse primers, 250 mM probe, and 50-100 ng/μL of DNA. After an initial activation/denaturation step at 50° C. for 2 min and then 95° C. for 10 min, amplification was performed during 40 cycles of 95° C.—15 seconds followed by 60° C.—1 minute. All mice in this study were confirmed to be HTT*Q97 carriers.

v. HTT mRNA Quantification

HTT mRNA quantification procedures were similar to those described in Example 8.

vi. HTT Protein Quantification

HTT protein quantification procedures were similar to those described in Example 8.

vii. Acceptance Criteria

Acceptance criteria were similar to those described in Example 8.

viii. Dose-Dependent Mutant Human HTT snRNA Knockdown by VY-HTT01

To evaluate the dose dependence of HTT mRNA lowering after intrastriatal dosing of VY-HTT01, human HTT and mouse XPNPEP1 mRNA (as an internal control) levels in striatum samples (a 3-mm punch from left hemibrain) were measured by RT-qPCR. All samples were run in duplicate for qPCR. Average Ct values and standard deviations (SD) were calculated for both HTT and XPNPEP1, and ΔCt (CtHTT-CtXPNPEP1) was determined for each sample. The ΔΔCt (ΔCt of VY-HTT01-injected samples-average vehicle ΔCT) was then calculated. Relative HTT mRNA levels were calculated for each sample by the 2−ΔΔCt method and expressed as the percentage relative to the vehicle control group. VY-HTT01 resulted in human HTT mRNA knockdown (KD) in the striatum at 4 weeks after intrastriatal injection in BACHD mice.

The average relative remaining HTT mRNA and relative HTT mRNA knockdown (KD) in YAC128 mouse striatum after administration with VY -HTT01 at different dose levels, as well as p-values generated by statistical analyses, are presented in Table 118.

TABLE 118
HTT mRNA lowering in BACHD striatum

| | | | Relative | | | | | |
|---|---|---|---|---|---|---|---|---|
| | | | remaining | | P value by | P value by | P value by | P value by |
| | | Total | HTT mRNA | HTT mRNA | one-way | one-way | one-way | one-way |
| | | dose | (% of veh; | KD (% of veh; | ANOVA | ANOVA | ANOVA | ANOVA |
| Group | Dose | (VG) | mean ± SD) | mean ± SD) | (vs Group 2) | (vs Group 3) | (vs Group 4) | (vs Group 5) |
| Group 1 | Veh | 0.0 | 100 ± 5 | 0 ± 5 | <0.0001 | <0.0001 | <0.0001 | <0.0001 |
| Group 2 | Low | 8.8e9 | 62 ± 13 | 38 ± 13 | — | <0.0001 | <0.0001 | <0.0001 |
| Group 3 | Mid | 2.8e10 | 42 ± 4 | 58 ± 4 | — | — | 0.0005 | 0.1686 |
| Group 4 | High | 8.8e10 | 28 ± 6 | 72 ± 6 | — | — | — | 0.1892 |
| Group 5 | MFD | 2.8e11 | 62 ± 13 | 65 ± 3 | — | — | — | — |

On average, 65±3% human HTT mRNA KD was observed at the maximal. feasible dose (p<0.0001 relative to vehicle group), 72±6% human HTT mRNA KD at the high dose (p<0.0001 relative to vehicle group), 58±4% human HTT mRNA KD at the middle dose (p<0.0001 relative to vehicle group) and 38±13% human HTT mRNA KD at the low dose (p<0.0001 relative to vehicle group). Dose-dependent human HTT mRNA KD was found with differences between HTT mRNA levels in the low-, middle- and high-dose groups, and in the low dose versus maximal feasible dose group. However, there was no difference in HTT mRNA levels between the MFD and high dose, nor between the. MFD and middle dose.

Together, these data demonstrated that VY-HTT01 suppressed striatal human HTT mRNA levels in a dose dependent manner in the BACHD mouse model of HD, and that maximal HTT mRNA lowering was attained at the high close (8.8×10¹⁰ VG per animal).

ix. Dose-Dependent Mutant Human HTT Protein Knockdown by VY-HTT01

To evaluate the dose dependence of HTT protein lowering after intrastriatal dosing of VY-HTT01, human HTT protein levels in right striatum tissue punches were measured by MSD. All samples were run in duplicate for MSD with the capture antibody 2B7 (a mouse monoclonal antibody against human HTT), and the detection antibody MW1 (an anti-polyQ specific antibody). Human HTT protein levels were calculated as human HTT protein concentration per mg of total protein (fmol/mg of protein) according to the standard curve. Relative HTT protein knockdown in each VY-HTT01-injected group was expressed as percentage of HTT protein in the vehicle control group. VY-HTT01 administration resulted in human mutant HTT protein KD in the striatum.

The average relative remaining HTT protein and relative HTT protein knockdown (KD) in BACHD mouse striatum after administration with VY-HTT01 at different dose levels, as well as p-values generated by statistical analyses, are presented in Table 119.

TABLE 119
HTT protein lowering in BACHD striatum

| | | | Relative | | | | | |
|---|---|---|---|---|---|---|---|---|
| | | | remaining | | P value by | P value by | P value by | P value by |
| | | Total | HTT protein | HTT protein | one-way | one-way | one-way | one-way |
| | | dose | (% of veh; | KD (% of veh; | ANOVA | ANOVA | ANOVA | ANOVA |
| Group | Dose | (VG) | mean ± SD) | mean ± SD) | (vs Group 2) | (vs Group 3) | (vs Group 4) | (vs Group 5) |
| Group 1 | Veh | 0.0 | 100 ± 6 | 0 ± 6 | p < 0.0001 | p < 0.0001 | p < 0.0001 | p < 0.0001 |
| Group 2 | Low | 8.8e9 | 55 ± 8 | 45 ± 8 | — | 0.3737 | p < 0.0001 | p < 0.0001 |
| Group 3 | Mid | 2.8e10 | 48 ± 13 | 52 ± 13 | — | — | 0.0006 | p < 0.0001 |
| Group 4 | High | 8.8e10 | 30 ± 7 | 70 ± 7 | — | — | — | 0.316 |
| Group 5 | MFD | 2.8e11 | 22 ± 5 | 78 ± 5 | — | — | — | — |

On average, human mutant HTT protein levels were suppressed by 78±5% at the AHD (p<0.0001), 70±7% at the high dose (p<0.0001), 52±13% at the middle dose (p<0.000) and 45±8% at the low dose (p<0.0001), at 4 weeks post dosing. This human HTT protein lowering was dose dependent, with differences in human HTT protein levels between the MFD versus middle (p<0.0001) or low dose (p<0.0001) groups, and high dose versus middle (p=0.0006) or low dose (p<0.0001) groups. There was no difference in human HTT protein levels between the MFD and high dose.

Together, these results demonstrated that -HTT01 suppressed human mutant HTT protein expression in a dose dependent manner in the BACHD mouse model of HD, and that at 4 weeks post-dose, maximal human HTT protein lowering in the striatum was attained at a dose of 8.8×10¹⁰ VG per animal.

This study demonstrated that intrastriatal injection of VY-HTT01 resulted in striatal human mutant HTT mRNA and protein KD in a dose dependent manner, ranging from 65-72% HTT mRNA and 70-78% HTT protein KD at the maximal feasible or high dose to 38% HTT mRNA and 45% protein KD at the low dose, at 4 weeks following injection. No abnormal cage side observations were detected in any animal in the study, Comparable body weight gain was seen for all groups and there were no differences in brain weight between vehicle- and VY HTT01-injected groups. These data demonstrated that VY-HTT01, administered by intrastriatal infusion, suppressed human mutant HTT mRNA and human mutant HTT protein levels in the BACHD mouse striatum in a dose dependent manner over 4 weeks, with maximal human mutant HTT protein lowering attained at a dose of 8.8×10¹⁰ VG per animal.

### Example 11

### In Vivo Study of VY-HTT01 Efficacy in the BACHD Mouse Model of Huntington's Disease

i. Test Article Preparation and Administration

The primary objective of this study was to investigate the efficacy of an AAV1 packaged viral genome, VOYHT1 (SEQ ID NO: 1352;hereinafter together referred to as AAV1-VOYHT1 or VY-HTT01) on motor function, general locomotion, and anxiety-like behavior over a 16-week period following bilateral intrastriatal infusion in the BACHD mouse, a model of Huntington's disease expressing human full-length mutant huntingtin (HTT). Further, this study was designed to evaluate the relationship between efficacy of VY-HTT01. administration on the above behavioral indices and VY-HTT01 dose, as well as HTT mRNA and protein lowering. Cage-side observations, and body and brain weight data, were also recorded.

On the basis of data obtained from dose response pharmacology experiments described in Example 10 (above), and in an effort to provide maximal and graded reduction of human mutant HTT protein, three dose levels of VY-HTT01 were selected for evaluation in the present study, as follows: 8.8×10¹⁰ VG/animal (high dose), 2.8×10¹⁰ VG/animal (middle or mid dose), and 8.8×10⁹ VG/animal (low dose). Briefly, data from Example 10 demonstrated 70%, 52%, and 45% human mutant HTT protein lowering, respectively, at these dose levels at 4 weeks after bilateral intrastriatal administration of VY-HTT01. In this study, brain tissues at the infusion site (striatum) were evaluated for human mutant HTT mRNA and protein levels at 16 weeks after administration (post-dosing).

This study involved forty-eight (48) female mils musculus FVB/N-Tg(HTT*97Q)IXwy/J-C57BL/6J (BACHD) mice and twelve (12) FVB/N-C57BL/6 (WT) mice (2-3 months old), randomly assigned into experimental groups by even distribution of age, body weight, and results from the baseline behavioral tests. On Day 1 all animals received bilateral injections of either vehicle (Groups 1 and 2) or VY-HTT01 (Groups 3-5) at 0.5 μL/minute into the left and right striata via a stereotaxically positioned silica catheter connected to a syringe. The details for groups, animal genotype, test articles and dosing paradigm are presented in Table 120.

TABLE 120
Study design

| | | | | Vector Dose | | | | |
|---|---|---|---|---|---|---|---|---|
| | | | | Volume | Dosing | | Total | |
| | | Number of | | injection | titer | | dose | In-life |
| Group | Genotype | animals | Dose | (μL/side) | (VG/mL) | VG/side | (VG) | (Weeks) |
| Group 1 | WT | 12 | Vehicle | 5 | 0.0 | 0.0 | 0.0 | 16 |
| Group 2 | BACHD | 12 | Vehicle | 5 | 0.0 | 0.0 | 0.0 | 16 |
| Group 3 | BACHD | 12 | VY-HTT01, | 5 | 8.8e11 | 4.4e9 | 8.8e9 | 16 |
| | | | low dose | | | | | |
| Group 4 | BACHD | 12 | VY-HTT01, | 5 | 2.8e12 | 1.4e10 | 2.8e10 | 16 |
| | | | mid dose | | | | | |
| Group 5 | BACHD | 12 | VY-HTT01, | 5 | 8.8e12 | 4.4e10 | 8.8e10 | 16 |
| | | | high dose | | | | | |

ii. Test Article Preparation and Administration

The test article preparation procedure was similar to that described in Example 8.

Animals were anesthetized by injection with ketamine (10 mg/mL) xylazine (1 mg/mL) and acepromazine (0.3 ma/mL) solution (160-200 μL/20 g) and placed into a stereotaxic frame. Intracranial injections were performed as follows: five microliters (5 μL) of recombinant viral vectors or vehicle were injected into the striatum (A/P +0.5, M/L ±2.0, DV −3.8 from bregma and dura; incisor bar, 0.0) using a 10 μL Hamilton syringe at the rate of 0.5 μL/min. The needle was left in place for 3 minutes following the completion of infusion, before it was raised out of the brain. Acetaminophen was provided in the drinking water 3 days before surgery and 3 days after surgery as pre- and post-operative analgesic.

iii. Cage-Side Observations, Body Weights, and Brain Weights

Cage-side observations were conducted prior to injection and performed daily starting after surgery. All mice were monitored for general health and signs of pain and/or illness daily.

For body weights, mice were weighed immediately prior to test article administration and weekly after dosing. Body weight measurements indicated that BACHD mice gain more weight over time than their WT littermates, However, there were no differences in body weight changes between BACHD groups dosed with vehicle or any dose of VY-HTT01 by one-way ANOVA with Tukey's post hoc test.

Brain weights were recorded after removal from the animal. Cage-side observations showed no abnormalities for all animals in all dose groups during the study. Brain weight measurements also showed no differences between BACHD groups dosed with vehicle or any dose of VY-HTT01 by one-way ANOVA.

Based on cage side observations, body weight gain, and brain weight, all doses of VY-HTT01 were well-tolerated over the 16-week in-life period of the study.

iv. In-Life Behavioral Tests

Four (4) behavioral tests were performed to collectively assess the effects of VY-HTT01 on BACHD mouse motor function and coordination, balance, locomotor activity, and anxiety-like behavior. These tests, described in detail below, were as follows: the accelerating rotarod test; the balance beam test; the open field test; and the light/dark box test.

The accelerating rotarod test was performed to assess the effects of VY-HTT01 on mouse motor function and motor coordination. BACHD mice were trained on the rotarod for two consecutive days (day 1 and 2) and tested on day 3. On the first training day, the rotarod was set to a constant speed of 5 RPM for 300 seconds, Mice that fell off the rod before completion of the 300-second time period were placed back on the rod until the full 300-second period expired. On the second day of training, the rotarod was set to accelerate from 5 to 40 rpm over 300 seconds. Mice that fell off the rod were placed back on the rod until it completed the 300-second training period. On day 3 (test day), the mice were placed on the rotarod set to accelerate from 5 to 40 rpm over 300 seconds. The latency to fall, defined by the time elapsed until the animal fell from the rotarod, was recorded over three trials. The rotarod test was carried out at pre-dosing for baseline measurement and was repeated every 4 weeks after dosing (day 28, 56, 84 and 113) until necropsy.

The balance beam test was performed to assess the effects of VY-HTT01 on mouse subtle motor coordination and balance. Before dosing, BACHD mice were trained on an elevated beam (1-meter long, 21-mm wide) on day 1, and were tested on a 1-meter long, 9-mm wide beam on day 2 for baseline measurement. The test was repeated on the 1-meter long, 9-nm wide beam at 8 weeks (day 57) and 16 weeks (day 114) after dosing. In each test, the latencies to traverse the beam (cross time) of two successful trials in which the mouse did not stall on the beam were averaged. The number of slips and falls were also recorded.

The open field test was performed at day 112 after dosing to assess locomotor activity and anxiety-like behavior. The test was carried out in a 27.3×27.3×20.3 cm white-base chamber. Mice were placed into the center of the test chamber and allowed to explore freely for 60 minutes. Locomotor activities including walking path and vertical movements were traced and measured using an overhead camera with a computer-assisted infra-red tracking system that computed total distance traveled (cm) and vertical counts. The percentage of time spent, and distance traveled in the center of the open field chamber, were also assessed for anxiety-like behavior.

The light/dark box test was conducted to assess the anxiety-like behavior at 8 weeks (day 58) and 16 weeks (day 115) after dosing. The apparatus used for the light/dark transition test consisted of the open field chamber with a black plastic insert to separate a dark compartment (dark zone) and a transparent compartment (light zone). Mice were placed in the center of the open field chamber, facing the dark compartment entrance and allowed to move freely between the dark and light compartments with the connecting gate open for 10 min. The distance traveled in each chamber, the total number of transitions, the time spent in each chamber, and the latency to enter the light chamber were recorded by a computer-assisted image program. The anxiety-like behavior was assessed by calculating the percentage of distance traveled in light zone (% distance travelled in light box) and the percentage of time spent in light zone (% time spent in light box).

Behavioral results were unblinded after the data had been collected and analyzed.

v. Tissue Collection

Sixteen weeks post VY-HTT01 dosing, mice received an intraperitoneal injection of ketamine (10 mg/mL), xylazine (1 mg/mL) and acepromazine (0.3 mg/mL) solution (160˜200 μL/20 g). Once under anesthesia, mice were weighed and body weights were recorded. The toe pinch-response method was used to determine depth of anesthesia. Once unresponsive, a subset of 8 mice per group were perfused transcardially with cold 1×PBS, and brains were carefully removed. Brain weights were recorded and then brains were cut along the coronal axis using a mouse brain matrix (Harvard Apparatus, Holliston, Mass.). Tissue samples were collected from the left and right striatum of a 2 mm-thick brain slab using a 3 mm biopsy punch. The overlying cortical region was also collected for potential biochemical analysis. These striatal and cortical samples were then flash-frozen in liquid nitrogen and stored at −80° C. for processing and mRNA or protein analysis.

The brains from the remaining 4 animals per group were immediately fixed with 4% paraformaldehyde after perfusion with 1×PBS for immunohistochemical analyses.

In addition, a 0.4 cm sample of tail from each mouse was collected and stored at −60° C. or lower until confirmatory genotyping analysis. In brief, for genotyping, the reaction mixture contained 900 mM of forward and reverse primers, 250 mM of probe, and 50-100 ng/uL of DNA. After an initial activation/denaturation step at 50° C. for 2 minutes and then 95° C. for 10 minutes, amplification was performed during 40 cycles of 95° C. for 15 seconds followed with 60° C. for 1 minute. Genotyping analysis of mouse tail snip samples confirmed that all 48 BACHD animals in the study were positive and all 12 WT mice were negative for the human mutant HTT gene, as expected.

vi. HTT mRNA Quantification by RT-qPCR

Striatal tissue punch processing, total RNA extraction and quantitative real-time PCR (RT-qPCR) were conducted using tissue punches collected from the left striatum. Human HTT and mouse X-Prolyl Aminopeptidase 1 (XPNPEP1)mRNA levels were determined by a RT-qPCR TaqMan assay, using procedures similar to those described in Example 8. HTT mRNA levels were normalized to XPNPEP1. mRNA levels, and then further normalized to the vehicle control group. RT-qPCR, results were unblinded after the data were analyzed.

vii. HTT Protein Quantification

HTT protein quantification procedures were similar to those described in Example 8.

viii. Immunohistochemical Analyses

Immunohistochemistry (IHC) staining for NeuN, GFAP, Iba1, DARPP32 and Fluoro-Jade C was conducted on mouse brains using standard procedures.

ix. Acceptance Criteria

Acceptance criteria were similar to those described in Example 8.

x. Effects of VY-HTT01 on BACHD Mouse Motor Function and Motor Coordination

As previously described, the accelerating rotarod test was performed to assess the effects of VY-HTT01 on motor function and motor coordination, while the balance beam test was performed to assess the effects of VY-HTT01 subtle motor coordination and balance. Data corresponding to these behavioral indices are presented in detail below.

The rotarod test was performed at pre-dosing and repeated every 4 weeks post-dosing up to 16 weeks post-dosing. The absolute latency to fall of each animal was recorded. Rotarod test absolute latency to fall (seconds) data are presented in Table 121.

TABLE 121
Latency to fall (seconds) in the rotarod test

| | | Pre-dose | 4-week | 8-week | 12-week | 16-week |
|---|---|---|---|---|---|---|
| | | baseline | post-dose | post-dose | post-dose | post-dose |
| | | (mean ± SD, | (mean ± SD, | (mean ± SD, | (mean ± SD, | (mean ± SD, |
| Genotype | Dose | n = 12/group) | n = 12/group) | n = 12/group) | n = 12/group | n = 23-12/group |
| WT | Vehicle | 277 ± 51 | 281 ± 16 | 242 ± 47 | 238 ± 46 | 236 ± 42 |
| BACHD | Vehicle | 182 ± 73 | 139 ± 41 | 105 ± 38 | 98 ± 34 | 90 ± 33 |
| BACHD | VY-HTT01, | 199 ± 67 | 113 ± 72 | 63 ± 35 | 50 ± 30 | 44 ± 31 |
| | high dose | | | | | |
| BACHD | VY-HTT01, | 181 ± 50 | 118 ± 36 | 85 ± 27 | 80 ± 29 | 73 ± 25 |
| | mid dose | | | | | |
| BACHD | VY-HTT01, | 189 ± 68 | 136 ± 32 | 107 ± 38 | 84 ± 33 | 86 ± 31 |
| | low dose | | | | | |

Latency to fall data were also expressed as percent (%) of pre-dose baseline (post-dosing latency/pre-doing latency×100%), or percent change in latency to fall. Rotarod test percent (%) latency to fall data are presented in Table 122.

TABLE 122
Percent (%) change in latency to fall in the rotarod test

| | | Pre-dose | 4-week | 8-week | 12-week | 16-week |
|---|---|---|---|---|---|---|
| | | baseline | post-dose | post-dose | post-dose | post-dose |
| | | (mean ± SD, | (mean ± SD, | (mean ± SD, | (mean ± SD, | (mean ± SD, |
| Genotype | Dose | n = 12/group) | n = 12/group) | n = 12/group) | n = 12/group | n = 23-12/group |
| WT | Vehicle | 100 ± 22 | 85 ± 25 | 94 ± 18 | 90 ± 23 | 91 ± 23 |
| BACHD | Vehicle | 100 ± 65 | 112 ± 44 | 103 ± 36 | 87 ± 35 | 91 ± 31 |
| BACHD | VY-HTT01, | 100 ± 59 | 110 ± 49 | 129 ± 41 | 121 ± 46 | 127 ± 53 |
| | high dose | | | | | |
| BACHD | VY-HTT01, | 100 ± 72 | 111 ± 58 | 118 ± 70 | 112 ± 61 | 128 ± 60 |
| | mid dose | | | | | |
| BACHD | VY-HTT01, | 100 ± 55 | 96 ± 51 | 102 ± 52 | 106 ± 50 | 124 ± 50 |
| | low dose | | | | | |

Together, BACHD mice had impaired motor function compared with WT littermates as indicated by a shorter latency to fall. No beneficial effects of VY-HTT01 at any dose were observed up to 16 weeks post-dosing, though VY-HTT01 high dose (8.8×10¹⁰ VG per animal) BACHD mice showed greater reduction in latency to fall versus vehicle-, VY-HTT01 middle- or low dose-BACHD mice by two-way ANOVA analysis with Tukey's multiple comparisons test. P values corresponding to statistical analyses performed on latency (% of pre-dose baseline), or percent change in latency to fall, rotarod data are presented in Table 123.

TABLE 123
Statistical analyses of percent (%) change in latency to fall in the rotarod test

| One-way ANOVA with | Pre-dose | 4-week | 8-week | 12-week | 16-week |
|---|---|---|---|---|---|
| Tukey's multiple comparisons | baseline | post-dose | post-dose | post-dose | post-dose |
| test (veh = vehicle; High = high | (mean ± | (mean ± | (mean ± | (mean ± | (mean ± |
| dose, Mid = mid dose, Low = | SD, n = | SD, n = | SD, n = | SD, n = | SD, n = |
| low dose VY-HTT01) | 12/group) | 12/group) | 12/group) | 12/group | 12/group |
| WT-Veh vs. BACHD-Veh | 0.016 | 0.044 | 0.032 | 0.067 | 0.004 |
| BACHD-Veh vs. BACHD-High | >0.999 | 0.408 | 0.017 | 0.003 | 0.004 |
| BACHD-Veh vs. BACHD-Mid | >0.999 | 0.595 | 0.457 | 0.561 | 0.545 |
| BACHD-Veh vs. BACHD-Low | >0.999 | 0.950 | 0.999 | 0.592 | 0.953 |
| BACHD-High vs. BACHD-Mid | >0.999 | 0.886 | 0.140 | 0.033 | 0.029 |
| BACHD-High vs. BACHD-Low | >0.999 | 0.565 | 0.020 | 0.047 | 0.009 |
| BACHD-Mid vs. BACHD-Low | >0.999 | 0.817 | 0.528 | >0.999 | 0.841 |

The balance beam test was performed at pre-dosing and then repeated at 8- and 16-weeks post-dosing to assess the effects of VY-HTT01 on subtle motor coordination and balance in BACHD mice. In each balance beam test, animals traversed an elevated 1-meter-long, 9-mm-wide beam for three trials. The absolute cross time (seconds) required to traverse the beam was recorded. The cross time as percent (%) of pre-dose baseline was calculated as post-dosing cross time/pre-dosing cross time×100%. Data on time spent to cross the balance beam, expressed as absolute cross time (seconds) are presented in Table 124.

TABLE 124
Time spent (seconds) to cross beam in the balance beam test

| | | Pre-dose | 8-week | 16-week |
|---|---|---|---|---|
| | | baseline | post-dose | post-dose |
| | | (mean ± | (mean ± | (mean ± |
| | | SD, n = | SD, n = | SD, n = |
| Genotype | Dose | 12/group) | 12/group) | 12/group) |
| WT | Vehicle | 10 ± 3 | 8 ± 2 | 7 ± 3 |
| BACHD | Vehicle | 16 ± 5 | 20 ± 8 | 18 ± 7 |
| BACHD | VY-HTT01, | 19 ± 7 | 19 ± 6 | 16 ± 7 |
| | high dose | | | |
| BACHD | VY-HTT01, | 22 ± 11 | 24 ± 17 | 14 ± 8 |
| | mid dose | | | |
| BACHD | VY-HTT01, | 17 ± 8 | 21 ± 9 | 15 ± 6 |
| | low dose | | | |

Data on time spent to cross the balance beam, expressed as a percent (%) of pre-dose baseline are presented in Table 125.

TABLE 125
Percent (%) change in time spent to
cross beam in the balance beam test

| | | Pre-dose | 8-week | 16-week |
|---|---|---|---|---|
| | | baseline | post-dose | post-dose |
| | | (mean ± | (mean ± | (mean ± |
| | | SD, n = | SD, n = | SD, n = |
| Genotype | Dose | 12/group) | 12/group) | 12/group) |
| WT | Vehicle | 100 ± 29 | 79 ± 21 | 70 ± 28 |
| BACHD | Vehicle | 100 ± 34 | 123 ± 50 | 113 ± 42 |
| BACHD | VY-HTT01, | 100 ± 34 | 101 ± 33 | 84 ± 39 |
| | high dose | | | |
| BACHD | VY-HTT01, | 100 ± 49 | 107 ± 75 | 65 ± 37 |
| | mid dose | | | |
| BACHD | VY-HTT01, | 100 ± 49 | 120 ± 54 | 87 ± 34 |
| | low dose | | | |

Together, BACHD mice injected with vehicle required more time to cross the balance beam at 16 weeks post-dosing versus WT mice, suggesting deficits in subtle motor coordination (p<0.05 by two-way ANOVA with Tukey's multiple comparisons test). In BACHD mice, mid-dose VY-HTT01 (2.8×10¹⁰ VG per animal) injection shortened cross time versus vehicle at 16 weeks post-dosing, providing support for VY-HTT01-associated improvement of subtle motor function and balance (p<0.05 by two-way ANOVA with Tukey's multiple comparisons test). However, cross time was unchanged with high-dose or low-dose VY-HTT01 (p>0.05).

The number of limb slips during transversal of the balance beam was also recorded. There were no changes in number of slips with any tested dose of VY-HTT01 by one-way ANOVA with Tukey's multiple comparisons test. Data on the number of slips in the balance beam test are presented in Table 126.

TABLE 126
Number of slips in the balance beam test

| | | Pre-dose | 8-week | 16-week |
|---|---|---|---|---|
| | | baseline | post-dose | post-dose |
| | | (mean ± | (mean ± | (mean ± |
| | | SD, n = | SD, n = | SD, n = |
| Genotype | Dose | 12/group) | 12/group) | 12/group) |
| WT | Vehicle | 0.2 ± 0.3 | 0.1 ± 0.2 | 0.1 ± 0.3 |
| BACHD | Vehicle | 0.3 ± 0.6 | 4.0 ± 4.8 | 3.2 ± 3.7 |
| BACHD | VY-HTT01, | 1.3 ± 2.8 | 6.1 ± 6.6 | 4.3 ± 5.2 |
| | high dose | | | |
| BACHD | VY-HTT01, | 0.3 ± 0.5 | 7.1 ± 7.6 | 6.0 ± 7.1 |
| | mid dose | | | |
| BACHD | VY-HTT01, | 0.6 ± 0.8 | 4.6 ± 3.6 | 4.6 ± 6.4 |
| | low dose | | | |

xi. Effects of VY-HTT01 on BACHD Mouse Locomotor Activity and Anxiety-Like Behavior

As previously described, the open field test was performed to assess locomotor activity and anxiety-like behavior. The light/dark box test was performed as an additional metric of anxiety-like behavior. Data corresponding to these behavioral indices are presented in detail below.

To measure general locomotor activity, the open field test was performed on BACHD mice and WT littermates at 16 weeks post-dosing (˜6.5 month of age). Briefly, each mouse was placed in the center of the open field chamber and allowed to freely explore the chamber for 60 minutes. Mouse locomotor activity was assessed by total distance (cm) travelled during 1-hour free exploration. Anxiety-like behavior was assessed by percent (%) time spent in the center zone of the open field and % distance travelled in the center zone of the open field. Data on the total distance travelled (cm), percent (%) time in center zone, and percent (%) distance traveled in the center zone of the open field test are presented in Table 127.

TABLE 127
Open field test locomotor activity and anxiety

| | | | Anxiety | |
|---|---|---|---|---|
| | | Locomotion | % center | |
| | | Total distance | distance | % center |
| | | (cm; mean ± | (mean ± | time (mean ± |
| | | SD, n = | SD, n = | SD, n = |
| Genotype | Dose | 12/group) | 12/group) | 12/group) |
| WT | Vehicle | 6054 ± 2248 | 44 ± 6 | 22 ± 9 |
| BACHD | Vehicle | 2385 ± 762 | 35 ± 10 | 13 ± 9 |
| BACHD | VY-HTT01, | 2486 ± 836 | 24 ± 14 | 7 ± 7 |
| | high dose | | | |
| BACHD | VY-HTT01, | 2965 ± 1597 | 30 ± 12 | 10 ± 6 |
| | mid dose | | | |
| BACHD | VY-HTT01, | 2096 ± 444 | 25 ± 11 | 10 ± 6 |
| | low dose | | | |

Together, vehicle-injected BACHD mice were hypoactive compared with WT littermates, as measured by the total distance travelled (p<0.0001, one-way ANOVA with Tukey's multiple comparisons test). Intrastriatal injection of VY-HTT01 had no effect at any dose on the total distance travelled in the open field test. Further, vehicle-injected BACHD mice showed increased anxiety versus WT littermates in the open field test at 16 weeks post-dosing as assessed by percent time spent in the center zone (p<0.05, one-way ANOVA with Tukey's multiple comparisons test), Intrastriatal injection of VY-HTT01 had no effect on open field test anxiety indices at 8- or 16-weeks post-dosing.

Anxiety-like phenotype was additionally assessed in the light/dark box test at 8 and 16 weeks post-dosing by evaluating percent (%) of total distance traveled (cm) in the light box, as well as percent (%) of total time spent in the light box. Data on percent (%) of total distance traveled (cm) in the light box, as well as percent (%) of total time spent in the light box for the light/dark box test are presented in Table 128.

TABLE 128
Percent (%) distance travelled and time in the light box in the light/dark box test

| | | 8 weeks post-dose | | 16 weeks post-dose | |
|---|---|---|---|---|---|
| | | % distance in | % time in | % distance | % time in |
| | | light box | light box | in light box | light box |
| | | (mean ± SD, | (mean ± SD, | (mean ± SD, | (mean ± SD, |
| Genotype | Dose | 12/group) | 12/group) | 12/group) | 12/group) |
| WT | Vehicle | 32.4 ± 3.8 | 35.7 ± 7.8 | 33.5 ± 5.8 | 36.4 ± 9.7 |
| BACHD | Vehicle | 22.3 ± 7.2 | 24.2 ± 11.5 | 25.1 ± 6.1 | 23.9 ± 10.1 |
| BACHD | VY-HTT01, | 18.7 ± 7.0 | 17.9 ± 11.1 | 27.8 ± 4.6 | 22.7 ± 6.7 |
| | high dose | | | | |
| BACHD | VY-HTT01, | 18.3 ± 6.7 | 18.4 ± 13.8 | 23.5 ± 7.3 | 19.1 ± 7.6 |
| | mid dose | | | | |
| BACHD | VY-HTT01, | 19.0 ± 6.3 | 17.3 ± 8.8 | 26.1 ± 6.9 | 21.7 ± 8.7 |
| | low dose | | | | |

Together, vehicle-injected BACHD mice showed increased anxiety in the light/dark box test at 8 and 16 weeks post-dosing as assessed by percent (%) of total distance traveled (cm) in the light box, as well as percent (%) of total time spent in the light box when compared with vehicle-injected SVT mice (p<0.05, one-way ANOVA with Tukey's multiple comparisons test). Intrastriatal injection of VY-HTT01 had no effect on these endpoints in the light/dark box test at 8- or 16-weeks post-dosing. Further, VY-HTT01 at the high-dose (8.8×10¹⁰ VG per animal) and low-dose (8.8×10⁹ VG per animal) increased % distance travelled in light box by 48.0% (p<0.01, two-way ANOVA Sidak's multiple comparisons test) and 36.5% (p<0.05, two-way ANOVA Sidak's multiple comparisons test), respectively, at 16 weeks versus 8 weeks post-dosing, suggesting that VY-HTT01 reduced anxiety over time.

xii. Dose-Dependent Human HTT n,RNA Knockdown by VY-HTT01

After completion of behavioral assessments at 16 weeks following intrastriatal dosing of VY-HTT01, the dose-dependence of human mutant HTT mRNA lowering was assessed in striatum samples from the left hemisphere of 8 animals per BACHD group. Human HTT and mouse XPNPEP1 mRNA (as an internal control) levels were measured by RT-qPCR. All samples were run in duplicate for qPCR. Average Ct values and standard deviations (SD) were calculated for both HTT and XPNPEP1, and ΔCt (CtHTT-CtXPNPEP1) was determined for each sample. The ΔΔCt (ΔCt of VY-HTT01-injected samples-average vehicle ΔCT) was then calculated. Relative human mutant HTT mRNA levels were calculated for each sample by the 2-ΔΔCt method and expressed as the percentage relative to the vehicle control BACHD group. All doses of VY-HTT01 resulted in human mutant HTT mRNA KD in the striatum at 16 weeks after intrastriatal injection in BACHD mice. Human HTT mRNA levels in BACHD mouse striatum at 16 weeks after VY-HTT01 administration at different dose levels, as well as p-values generated by statistical analyses, are presented in Table 129.

TABLE 129
HTT mRNA lowering in BACHD striatum at 16 weeks post dose

| | | 16 weeks post dose | | | | | |
|---|---|---|---|---|---|---|---|
| | | (% of Veh; mean ± SD, | | | | | |
| | | n = 8/group) | | P value by | P value by | P value by | P value by |
| | | Relative | | one-way | one-way | one-way | one-way |
| | | remaining | HTT | ANOVA | ANOVA | ANOVA | ANOVA |
| Genotype | Dose | HTT mRNA | mRNA KD | (vs Veh) | (vs High) | (vs Mid | (vs Low) |
| BACHD | Vehicle | 100 ± 26 | 0 ± 26 | — | <0.0001 | <0.0001 | <0.0001 |
| BACHD | VY-HTT01, | 55 ± 10 | 45 ± 10 | — | — | 0.9992 | 0.4248 |
| | high dose | | | | | | |
| BACHD | VY-HTT01, | 54 ± 10 | 46 ± 10 | — | — | — | 0.4994 |
| | mid dose | | | | | | |
| BACHD | VY-HTT01, | 43 ± 7 | 57 ± 7 | — | — | — | — |
| | low dose | | | | | | |

On average, there was 45±10% human mutant HTT mRNA KD at the high dose (p<0.0001 relative to the BACHD vehicle group), 46±10% human mutant HTT mRNA KD at the middle dose (p<0.0001 relative to the RACED vehicle group) and 57±7% human mutant HTT mRNA KD at the low dose (p<0.0001 relative to the BACHD vehicle group). There was no difference in HTT mRNA levels across high, middle and low dose BACHD groups at 16 weeks post-dose. Thus, intrastriatal administration of VY-HTT01 at 8.8×10⁹, 2.8×10¹⁰ and 8.8×10¹⁰ VG per animal results in similar 45-57% striatal human mutant HTT mRNA suppression in BACHD mice at 16 weeks post-dose.

After completion of behavioral assessments at 16 weeks following intrastriatal dosing of VY-HTT01, the dose-dependence of human mutant HTT protein lowering was assessed in striatum samples from the right hemisphere of 8 animals per group using the MSD assay. All doses of VY-HTT01 resulted in human mutant HTT protein KD in the striatum. Human HTT protein levels in BACHD mouse striatum at 16 weeks after VY-HTT01 administration at different dose levels, as well as p-values generated by statistical analyses, are presented in Table 130.

TABLE 130
HTT protein lowering in BACHD striatum at 16 weeks post dose

| | | 16 weeks post dose | | | | | |
|---|---|---|---|---|---|---|---|
| | | (% of Veh; mean ± SD, | | | | | |
| | | n = 8/group) | | P value by | P value by | P value by | P value by |
| | | Relative | | one-way | one-way | one-way | one-way |
| | | remaining | HTT | ANOVA | ANOVA | ANOVA | ANOVA |
| Genotype | Dose | HTT protein | protein KD | (vs Veh) | (vs High) | (vs Mid | (vs Low) |
| BACHD | Vehicle | 100 ± 23 | 0 ± 23 | — | <0.0001 | <0.0001 | <0.0001 |
| BACHD | VY-HTT01, | 23 ± 3 | 77 ± 3 | — | — | 0.2745 | 0.0003 |
| | high dose | | | | | | |
| BACHD | VY-HTT01, | 34 ± 7 | 66 ± 7 | — | — | — | 0.0295 |
| | mid dose | | | | | | |
| BACHD | VY-HTT01, | 53 ± 8 | 47 ± 8 | — | — | — | — |
| | low dose | | | | | | |

On average, human mutant HTT protein levels were suppressed by 77±3% at the high dose (p<0.0001), 66±7% at the middle dose (p<0.0001) and 47±8% at the low dose (p<0.0001), at 16 weeks post-dosing using a one-way ANOVA with Tukey's post-hoc test. This human mutant HTT protein lowering was dose-dependent, with differences in human HTT protein levels between the high versus low dose (p<0.001) and middle versus low dose (p<0.05) groups. There was no difference in human mutant HTT protein level between the high and middle dose VY-HTT01 groups, These results demonstrated that intrastriatal dosing of VY-HTT01 suppressed human mutant HTT protein expression in the striatum in a dose-dependent manner in the BACHD mouse model of HD, and that maximal human mutant HTT protein lowering in the striatum was attained at 2.8×10¹⁰ VG per animal at 16 weeks post-dose.

xiii. Immunohistochemical Analyses of Cellular Markers Following Intrastriatal Dosing with VY-HTT01

After completion of behavioral assessments at 16 weeks following intrastriatal dosing of VY-HTT01, the brains from 4 animals per group were used for immunohistochemistry. Immunohistochemical (IHC) analyses were performed to investigate whether VY-HTT01 affects NeuN, DARPP32, Fluoro-Jade C, GFAP and Iba1 staining in the BACHD mouse striatum.

There were no differences in striatal density of NeuN-positive cells between vehicle BACHD and WT mice, or across vehicle and VY-HTT01 BACHD mice. Further, there were no differences in DARPP32, Iba1, or GFAP immunoreactivity in the striatum between vehicle-injected BACHD and WI mice, nor across vehicle and VY-HTT01 BACHD mice. Neuronal degeneration was not identified in any group, based on Fluoro-Jade C immunoreactivity. s.

In this study, no treatment-related differences in neuronal cell density, gliosis, or body weight were identified in BACHD mice.

This study demonstrated that intrastriatal injection of VY-HTT01 (8.8×10⁹, 2.8×10¹⁰ or 8.8×10¹⁰ VG per animal) resulted in striatal human mutant HTT mRNA KD ranging from 45% to 57%, accompanied by striatal human mutant HTT protein KD by 47-77% that was dose-dependent at 16 weeks post-dose in BACHD mice. VY-HTT01 (2.8×10¹⁰ VG per animal) improved motor coordination deficits in the balance beam test at 16 weeks post-dose in BACHD mice. VY-HTT01 (8.8×10⁹ and 8.8×10¹⁰ VG per animal) improved anxiety in the light/dark box test between 8- and 16-weeks post-dose in BACHD mice.

The high dose of VY-HTT01 (8.8×10¹⁰ VG per animal) resulted in HTT protein KD by 77% in the striatum; a reduced latency to fall in the rotarod test at 16 weeks post-dose; and, improved anxiety in the light/dark box test from test 8 to 16 weeks post-dose in BACHD mice compared with BACHD mice administered vehicle. The middle (mid) dose of VY-HTT01 (2.8×10¹⁰ VG per animal) resulted in HTT protein KD by 66% in the striatum, as well as improved subtle motor coordination in the balance beam test at 16 weeks post-dose compared with BACHD administered vehicle. The low dose of VY-HTT01 (8.8×10⁹ VG per animal) resulted in human mutant HTT protein KD by 47% in the striatum accompanied by improved anxiety-like behavior in the light/dark box test from 8 to 16 weeks post-dose compared with BACHD mice administered vehicle VY-HTT01 had no apparent effects on neuronal or glial markers.

These data demonstrate that VY-HTT01 administered by intrastriatal infusion lowers human mutant HTT expression in the BACHD mouse striatum in a dose-dependent manner, and improved subtle motor coordination as assessed by the balance beam test at the middle (2.8×10¹⁰ VG per animal) dose of VY-HTT01, and improved anxiety in BACHD mice, as assessed by the light/dark box test at the low (8.8×10⁹ VG per animal) and high (8.8×10¹⁰ VG per animal) doses of VY-HTT01.

### Example 12

### HTT Protein Evaluation in NHP Putamen, Caudate, and Thalamus after Intraputaminal and Intrathalamic Infusion of VY-HTT01 With Different Delivery Parameters

I. Study Design

As an extension of Example 1 (Dose Optimization Study I), described in detail above, the overarching goal of this study was to measure HTT protein levels in tissue punches from the putamen, caudate and thalamus 5 weeks after bilateral combined intraputaminal and intrathalamic infusion of AAV1-VOYHT1 in non-human. primate (NHP) rhesus macaque (Macaca mulatto), toward characterizing the effects of varying AAV1-VOYHT1 delivery parameters (e.g., volume of infusion and concentration) on HTT protein levels, and as a basis for establishing; future dosing parameters. NHP HTT mRNA was also measured from the same tissue samples in order to interrogate the relationship between relative HTT protein and relative HTT mRNA levels from the same sample. General aspects of study implementation, including animal care/groups, test article preparation, and dosing schedules, were the same as described in Example 1 (above). The study design summarized in Table 40 similarly reflects that of the present study.

ii. Tissue Sample Collection and Preparation

Approximately twenty-two coronal brain slices (3 mm thick) were collected for each animal using a monkey brain matrix. Brain slices were then fresh frozen and stored at −60° C. for potential pharmacology analysis or were fixed for histopathology evaluation.

Procedures for brain tissue sample collection for subsequent quantitation of HTT protein were similar to those described in Example 7 (above). Briefly, frozen brain slices were warmed at room temperature (for ˜1 min), and then tissue samples were rapidly collected using a 2 mm biopsy punch outfitted with a plunger, and immediately transferred to a pre-cooled collection tube on dry ice. All tissue punches were maintained on dry ice during collection and then stored at −80° C. until tissue analysis.

Procedures for tissue sample preparation for subsequent quantitation of HTT protein were as described in Example 7 (above). Briefly, brain tissue punches were weighed, pulverized using a tissue impactor, transferred into pre-chilled collection bottle, and stored on dry ice. The pulverized dry tissue powder was aliquoted into two parts: one quarter of the tissue powder was transferred to a new pre-chilled tube for HTT mRNA measurement by the branched DNA (bDNA) assay, and the remaining three-quarters of tissue powder was transferred into another pre-chilled tube for HTT protein quantification by LC-MS/MS.

iii. Branched DNA Assay for Quantitation HTT mRNA

Methods for quantitation of HTT mRNA by branched DNA (bDNA) assay were as described above in Example 1 (above).

iv. LC-MS/MS for Quantitation of HTT Protein

Methods for quantitation of HTT protein by UPLC-MS/MS (Ultra performance liquid chromatography-mass spectrometry and liquid chromatography-tandem mass spectrometry), or LC-MS/MS, were as described above in Example 7 (above). All samples were run at the same time for HTT protein quantification by LC-MS/MS.

v. Putamen HTT Protein and mRNA Levels

To evaluate HTT protein and mRNA lowering in the putamen, 4 punches were collected from the putamen in the right hemisphere of all animals from all groups, resulting in a total of 12 punches per group. Aliquots of pulverized putamen punch powder were used for HTT and beta-actin protein analysis by LC-MS/MS. HTT protein levels were normalized to levels of beta-actin (internal control), and then further normalized to the vehicle group. The average relative remaining putamen HTT protein and relative putamen HTT protein knockdown (KD) across total VG doses (VG/animal, or VG) and expressed as a percentage of vehicle (veh), corresponding standard deviation (stdev) values, as well as P-values generated by one-way ANOVA with. Tukey's multiple comparisons, are presented in Table 131. Note that each group (grp) of animals listed in in the below Table 131 consisted of 3 animals per group, with the exception of group A3, which had only 2 animals per group.

TABLE 131
Dose-dependent HTT protein lowering in the NHP putamen after bilateral
intraputaminal and intrathalamic infusion of AAV1-VOYHT1

| | | Relative | Relative | P value by | P value by | P value by | P value by | P value by |
|---|---|---|---|---|---|---|---|---|
| | Total | remaining | HTT | one-way | one-way | one-way | one-way | one-way |
| Group | dose | HTT protein | protein KD | ANOVA | ANOVA | ANOVA | ANOVA | ANOVA |
| (Grp) | (VG) | (% veh ± stdev) | (% veh ± stdev) | (vs Grp A1) | (vs Grp A2) | (vs Grp A3) | (vs Grp A4) | (vs Grp A5) |
| Grp A6 | 0.00 | 100 ± 4 | 0 ± 4 | 0.2951 | 0.0006 | 0.0023 | 0.2259 | 0.8209 |
| Grp A5 | 1.35e11 | 90 ± 8 | 10 ± 8 | 0.9014 | 0.0033 | 0.012 | 0.8217 | — |
| Grp A4 | 4.50e11 | 79 ± 19 | 21 ± 19 | >0.9999 | 0.0221 | 0.068 | — | — |
| Grp A1 | 6.75e11 | 81 ± 11 | 19 ± 11 | — | 0.0162 | 0.0516 | — | — |
| Grp A2 | 1.35e12 | 46 ± 4 | 54 ± 4 | — | — | 0.9997 | — | — |
| Grp A3 | 2.16e12 | 48 ± 4 | 52 ± 4 | — | — | — | — | — |

Significant and dose-dependent HTT protein reduction was achieved in the putamen after bilateral intraputaminal and intrathalamic infusion of different total doses of AAV1-VOYHT1. Specifically, on average, there was 10%, 21%, 19%, 54% and 52% HTT protein KD for group A5 (1.35×10¹¹ VG per animal), group A4 (4.50×10¹¹ VG per animal), group A1 (6.75×10¹¹ VG per animal), group A2 (1.35×10¹² VG per animal) and group A3 (2.16×10¹² VG per animal), respectively, relative to the vehicle (veh) group (group A6), At the two highest doses of 1.35×10¹² and 2.16×10¹² VG per animal, HTT protein KD was statistically significant (P<0.01 by one-way ANOVA with Tukey's multiple comparisons test) relative to vehicle (group A6) and statistically greater (P<0.05 by one-way ANOVA with Tukey's multiple comparisons test) than the lowest dose group (1.35×10¹¹ VG per animal). The two highest doses (1.35×10¹² and 2.16×10¹² VG per animal) also resulted in HTT protein KD that was statistically greater (P<0.05 by one-way ANOVA with Tukey's multiple comparisons test) than the HTT protein KD resulting from the 4.50×10¹¹ and 6.75×10¹¹ VG per animal doses. There was no statistical difference between the HTT protein KD at the two highest doses (1.35×10¹² and 2.16×10¹² VG per animal) (P=0.9997 by one-way ANOVA with Tukey's multiple comparisons test).

The effect of infusion volume on HTT protein lowering in the putamen was then evaluated at the highest vector concentration (2.7×10¹² vg/mL) for groups A1 (putamen/50 μL, thalamus/75 μL), A2 (putamen/100 μL, thalamus/150 μL), and A3 (putamen/150 thalamus/250 μL). The average relative remaining putamen HTT protein and relative putamen HTT protein knockdown (KD) across infusion volumes and expressed as a percentage of vehicle (veh), corresponding standard deviation (stdev) values, as well as P-values generated by one-way ANOVA with Tukey's multiple comparisons, are presented in Table 132. Note that each group (grp) of animals listed in Table 132 consisted of 3 animals per group, with the exception of group A3, which had only 2 animals per group.

TABLE 132
Volume-dependent HTT protein lowering in the NHP putamen after bilateral
intraputaminal and intrathalamic infusion of AAV1-VOYHT1

| | | Relative | Relative | P value by | P value by | P value by |
|---|---|---|---|---|---|---|
| | Total | remaining | HTT | one-way | one-way | one-way |
| Group | dose | HTT protein | protein KD | ANOVA | ANOVA | ANOVA |
| (Grp) | (VG) | (% veh ± stdev) | (% veh ± stdev) | (vs Grp A1) | (vs Grp A2) | (vs Grp A3) |
| Grp A6 | 0.00 | 100 ± 4 | 0 ± 4 | 0.0454 | 0.0001 | 0.0004 |
| Grp Al | 6.75e11 | 81 ± 11 | 19 ± 11 | — | 0.0019 | 0.0057 |
| Grp A2 | 1.35e12 | 46 ± 4 | 54 ± 4 | — | — | 0.9707 |
| Grp A3 | 2.16e12 | 48 ± 4 | 52 ± 4 | — | — | — |

Bilateral combined intraputaminal and intrathalamic infusion of AAV1-VOYHT1 resulted in substantial HTT protein KD in the putamen in a volume-dependent manner at the highest vector concentration (2.7×10¹² vg/mL). An average HTT protein KD of 52%, 54% and 19% was achieved in the putamen for group A3 (putamen/150 μL, thalamus/250 μL), group A2 (putamen/100 μL, thalamus/150 μL) and group A1 (putamen/50 μl, thalamus/75 μL), respectively, that was statistically significant (P<0.05) versus vehicle (group A6), with higher volumes of infusion used for groups A2 and A3 resulting in greater HTT protein KD (P<0.05) than the lowest volume in group A1. There was no statistically significant difference (P=0.9707) between the volumes of infusion used for groups A2 (putamen/100 μL, thalamus/150 μL) and A3 (putamen/150 μL, thalamus/250 μL).

The effect of vector concentration on HTT protein KD in the putamen of NHP after bilateral intraputaminal and intrathalamic administration of AAV1-VOYHT1 at the same dosing volume (putamen/100 μL, thalamus/150 μL) was also evaluated for group A5 (2.7×10¹¹ vg/mL), group A4 (9.0×10¹¹ vg/mL) and group A2 (2.7×10¹² vg/mL) versus the vehicle control group (group A6). The average relative remaining putamen HTT protein and relative putamen HTT protein knockdown (KD) across vector concentrations and expressed as a percentage of vehicle (veh), corresponding standard deviation (stdev) values, as well as P-values generated by one-way ANOVA with Tukey's multiple comparisons, are presented in Table 133. Note that each group (grp) of animals listed in Table 132 consisted of 3 animals per group.

TABLE 133
Concentration-dependent HTT protein lowering in the NHP putamen after
bilateral intraputaminal and intrathalamic infusion of AAV1-VOYHT1

| | | Relative | Relative | P value by | P value by | P value by |
|---|---|---|---|---|---|---|
| | Total | remaining | HTT | one-way | one-way | one-way |
| | dose | HTT protein | protein KD | ANOVA | ANOVA | ANOVA |
| Group | (VG) | (% veh ± stdev) | (% veh ± stdev) | (vs Grp A5) | (vs Grp A4) | (vs Grp A2) |
| Grp A6 | 0.00 | 100 ± 4 | 0 ± 4 | 0.6537 | 0.1626 | 0.0012 |
| Grp A5 | 1.35e11 | 90 ± 8 | 10 ± 8 | — | 0.6545 | 0.0047 |
| Grp A4 | 4.50e11 | 79 ± 19 | 21 ± 19 | — | — | 0.0215 |
| Grp A2 | 1.4e12 | 46 ± 4 | 54 ± 4 | — | — | — |

These results show that at the same dosing volume (putamen/100 μL, thalamus/150 μL), an average of 54%, 21% and 10% HTT protein KD was achieved in the putamen for group A2 (2.7×10¹² vg/mL), group A4 (9.0×10¹¹ vg/mL) and group A5 (2.7×10¹¹ vg/mL), respectively. Further, the highest HTT protein KD was achieved in group A2 at the highest AAV1-VOYHT1 concentration 2.7×1.0¹² vg/mL), which was statistically significant (P<0.05) relative to vehicle and relative to the two lower AAV1-VOYHT1 concentrations of 9.0×10¹¹ vg/mL and 2.7×10¹¹ vg/mL.

NHP HTT mRNA levels in the putamen were also measured by the bDNA assay, as described in Example 1 (above), in the same putamen punches used for HTT protein quantitation, using a specific probe set against rhesus HTT, TBP, AARS and XPNPEP1. HTT mRNA levels were normalized to the geometric mean of the three housekeeping genes TBP, AARS and XPNPEP1, and then further normalized to the vehicle group. The average relative remaining putamen HTT mRNA and relative putamen HTT mRNA knockdown (KD), across VG doses (VG/animal, or VG) and expressed as a percentage of vehicle (veh), corresponding standard deviation (stdev) values, as well as P-values generated by one-way ANOVA with Tukey's multiple comparisons, are presented in Table 134. Note that each group (grp) of animals listed in Table 134 consisted of 3 animals per group, with the exception of group A3, which had only 2 animals per group.

TABLE 134
Dose-dependent HTT mRNA lowering in the NHP putamen after bilateral
intraputaminal and intrathalamic infusion of AAV1-VOYHT1

| | | Relative | Relative | P value by | P value by | P value by | P value by | P value by |
|---|---|---|---|---|---|---|---|---|
| | Total | remaining | HTT | one-way | one-way | one-way | one-way | one-way |
| Group | dose | mRNA protein | mRNA KD | ANOVA | ANOVA | ANOVA | ANOVA | ANOVA |
| (Grp) | (VG) | (% veh ± stdev) | (% veh ± stdev) | (vs Grp A1) | (vs Grp A2) | (vs Grp A3) | (vs Grp A4) | (vs Grp A5) |
| Grp A6 | 0.00 | 100 ± 6 | 0 ± 6 | 0.0928 | 0.0013 | 0.0011 | 0.0217 | 0.2672 |
| Grp A5 | 1.35e11 | 77 ± 7 | 23 ± 7 | 0.9766 | 0.0454 | 0.0251 | 0.6037 | — |
| Grp A4 | 4.50e11 | 61 ± 21 | 39 ± 21 | 0.9363 | 0.4755 | 0.2386 | — | — |
| Grp A1 | 6.75e11 | 70 ± 10 | 30 ± 10 | — | 0.1391 | 0.0695 | — | — |
| Grp A2 | 1.35e12 | 43 ± 14 | 57 ± 14 | — | — | 0.9728 | — | — |
| Grp A3 | 2.16e12 | 34 ± 3 | 66 ± 3 | — | — | — | — | — |

A significant and dose-dependent HTT mRNA reduction was achieved in the putamen after bilateral intraputaminal and intrathalamic infusion of different total doses of AAV1-VOYHT1. Specifically, on average, there was 23%, 39%, 30%, 57% and 66% HTT mRNA. KD for group A5 (1.35×10¹¹ VG per animal), group A4 (4.50×10¹¹ VG per animal), group A1 (6.75×10¹¹ VG per animal), group A2 (1.35×10¹² VG per animal) and group A3 (2.16×10¹² VG per animal), respectively, relative to the vehicle group (group A6). At the two highest doses of 1.35×10¹² VG per animal and 2.16×10¹² VG per animal and at the dose of 4.50×10¹¹ VG per animal HTT mRNA KD was statistically significant (P<0.05 by one-way ANOVA with Tukey's multiple comparisons test) relative to vehicle (group A6). In addition, the two highest doses resulted in HTT mRNA KD that was statistically greater (P<0.05 by one-way ANOVA. with Tukey's multiple comparisons test) than the lowest dose group (1.35×10¹¹ VG per animal). There was no statistical difference between the HTT mRNA KD at the two highest doses (1.35×10¹² and 2.16×10¹² VG per animal) (P=0.9728 by one-way ANOVA with Tukey's multiple comparisons test).

The effect of vector volume on HTT mRNA lowering in the putamen was then evaluated at the highest vector concentration (2.7×10¹² vg/mL) for groups A1 (putamen/50 μL, thalamus/75 μL), A2 (putamen/100 μL, thalamus/150 μL), and A3 (putamen/150 μL, thalamus/250 μL). The average relative remaining putamen HTT mRNA and relative putamen HTT mRNA knockdown (KD) across infusion volumes and expressed as a percentage of vehicle, corresponding standard deviation (stdev) values, as well as P-values generated by one-way ANOVA with Tukey's multiple comparisons, are presented in Table 135. Note that each group (grp) of animals listed in Table 135 consisted of 3 animals per group, with the exception of group A3, which had only 2 animals per group.

TABLE 135
Volume-dependent HTT mRNA lowering in the NHP putamen after bilateral
intraputaminal and intrathalamic infusion of AAV1-VOYHT1

| | | Relative | Relative | P value by | P value by | P value by |
|---|---|---|---|---|---|---|
| | Total | remaining | HTT | one-way | one-way | one-way |
| Group | dose | HTT mRNA | mRNA KD | ANOVA | ANOVA | ANOVA |
| (Grp) | (VG) | (% veh ± stdev) | (% veh ± stdev) | (vs Grp A1) | (vs Grp A2) | (vs Grp A3) |
| Grp A6 | 0.00 | 100 ± 6 | 0 ± 6 | 0.0303 | 0.0009 | 0.0007 |
| Grp A1 | 6.75e11 | 70 ± 10 | 30 ± 10 | — | 0.0456 | 0.0229 |
| Grp A2 | 1.35e12 | 43 ± 14 | 57 ± 14 | — | — | 0.8043 |
| Grp A3 | 2.16e12 | 34 ± 3 | 66 ± 3 | — | — | — |

An average HTT mRNA KD of 66%, 57% and 30% was achieved in the putamen for group A3 (putamen/150 μL. thalamus/250 μL), group A2 (putamen/100 μL, thalamus/150 μL) and group A1 (putamen/50 μL, thalamus/75 μL), respectively, that was statistically significant (P<0.05 by one-way ANOVA with Tukey's multiple comparison test) versus vehicle (group A6), with the higher volumes of infusion in groups A2 and A3 resulting in statistically greater HTT mRNA KD (P<0.05 by one-way ANOVA with Tukey's multiple comparison test) than the lowest volume in group A1. There is no statistical difference between HTT mRNA KD in groups A2 and A3 (P =0.8043 by one-way ANOVA with Tukey's multiple comparison test). There was no statistical difference between HTT mRNA KD in groups A2 and A3 (P=0.8043 by one-way ANOVA with Tukey's multiple comparison test).

The effect of vector concentration on HTT mRNA KD in the putamen of NHP after bilateral intraputaminal and intrathalamic administration of AAV1-VOYHT1 at the same dosing volume (putamen/100 μL, thalamus/150 μL) was also evaluated for group A5 (2.7×10¹¹ vg/mL), group A4 (9.0×10¹¹ vg/mL) and group A2 (2.7×10¹² vg/mL) versus the vehicle control group (group A6). The average relative remaining putamen HTT mRNA and relative putamen HTT mRNA knockdown (KD) across vector concentrations and expressed as a percentage of vehicle (veh), corresponding standard deviation (stdev) values, as well as P-values generated by one-way ANOVA with Tukey's multiple comparisons, are presented in Table 136, Note that each group (grp) of animals listed in in the below Table 136 consisted of 3 animals per group.

TABLE 136
Concentration-dependent HTT mRNA lowering in the NHP putamen after
bilateral intraputaminal and intrathalamic infusion of AAV1-VOYHT1

| | | Relative | Relative | P value by | P value by | P value by |
|---|---|---|---|---|---|---|
| | Total | remaining | HTT | one-way | one-way | one-way |
| | dose | mRNA protein | mRNA KD | ANOVA | ANOVA | ANOVA |
| Group | (VG) | (% veh ± stdev) | (% veh ± stdev) | (vs Grp A5) | (vs Grp A4) | (vs Grp A2) |
| Grp A6 | 0.00 | 100 ± 6 | 0 ± 6 | 0.2284 | 0.0291 | 0.0033 |
| Grp A5 | 1.35e11 | 77 ± 7 | 23 ± 7 | — | 0.4906 | 0.0523 |
| Grp A4 | 4.50e11 | 61 ± 21 | 39 ± 21 | — | — | 0.3871 |
| Grp A2 | 1.4e12 | 43 ± 14 | 57 ± 14 | — | — | — |

These results show that at the same dosing volume (putamen/100 μL, thalamus/150 μL), an average of 57%, 39% and 23% HTT protein KD was achieved in the putamen for group A2 (2.7×10¹² vg/mL), group A4 (9.0×10¹¹ vg/mL) and group A5 (2.7×10¹¹ vg/mL), respectively. The highest HTT mRNA KD was achieved at the two highest concentrations (2.7×10¹² vg/mL, group A2 and 4.50×10¹¹ vg/mL, group A4), which were statistically significant (P<0.05 by one-way ANOVA with Tukey's multiple comparison test) relative to vehicle (group A6). There was no statistically significant difference in HTT snRNA KD between different AAV1-VOYHT1 concentrations, although the lowest (2.7×10¹¹ vg/mL) and highest (2.7×10¹² vg/mL) concentrations trended toward different (P=0.0523 by one-way ANOVA with Tukey's multiple comparison test).

vi. Caudate NTT Protein and mRNA Levels

To evaluate HTT protein and mRNA lowering in the caudate, 4 punches were collected from the caudate from each animal (2 per hemisphere), resulting in a total of 12 punches per group. Aliquots of pulverized caudate punch powder were used for HTT and beta-actin protein analysis by LC-MS/MS. HTT protein levels were normalized to levels of beta-actin (internal control), and then further normalized to the vehicle group. The average relative remaining caudate HTT protein and relative caudate HTT protein knockdown (KD) across total VG doses (VG/animal, or VG) and expressed as a percentage of vehicle (veh), corresponding standard deviation (stdev) values, as well as P-values generated by one-way ANOVA with Tukey's multiple comparisons, are presented in Table 137. Note that each group (grp) of animals listed in Table 137 consisted of 3 animals per group.

TABLE 137
HTT protein levels in the NHP caudate after bilateral intraputaminal and intrathalamic infusion of AAV1-VOYHT1

| | | Relative | Relative | P value by | P value by | P value by | P value by | P value by |
|---|---|---|---|---|---|---|---|---|
| | Total | remaining | HTT | one-way | one-way | one-way | one-way | one-way |
| Group | dose | HTT protein | protein KD | ANOVA | ANOVA | ANOVA | ANOVA | ANOVA |
| (Grp) | (VG) | (% veh ± stdev) | (% veh ± stdev) | (vs Grp A1) | (vs Grp A2) | (vs Grp A3) | (vs Grp A4) | (vs Grp A5) |
| Grp A6 | 0.00 | 100 ± 4 | 0 ± 4 | 0.8607 | 0.4446 | 0.1858 | 0.502 | 0.9995 |
| Grp A5 | 1.35e11 | 98 ± 11 | 2 ± 11 | 0.9587 | 0.6179 | 0.2914 | 0.679 | — |
| Grp A4 | 4.50e11 | 87 ± 11 | 13 ± 11 | 0.9823 | >0.9999 | 0.9714 | — | — |
| Grp A1 | 6.75e11 | 92 ± 2 | 8 ± 2 | — | 0.9667 | 0.718 | — | — |
| Grp A2 | 1.35e12 | 86 ± 9 | 14 ± 9 | — | — | 0.9853 | — | — |
| Grp A3 | 2.16e12 | 82 ± 11 | 18 ± 11 | — | — | — | — | — |

On average, there was 2%, 13%, 8%, 14% and 18% HTT protein KD for group A5 (1.35×10¹¹ VG per animal), group A4 (4.5×10¹¹ VG per animal), group A1 (total 6.75×10¹¹ VG per animal), group A2 (total 1.35×10¹² VG per animal) and group A3 (2.16×10¹² VG per animal), respectively, relative to the vehicle group (group A6). These reductions in HTT protein were not statistically significant (P>0.05 by one-way ANOVA with Tukey's multiple comparisons test).

NHP HTT mRNA levels in the caudate were also measured by the bDNA assay, as described in Example 1 (above), in the same caudate punches used for HTT protein quantitation, using a specific probe set against rhesus HTT, TBP, AARS and XPNPEP1. HTT mRNA levels were normalized to the geometric mean of the three housekeeping genes TBP, AARS and XPNPEP1, and then further normalized to the vehicle group. The average relative remaining caudate HTT mRNA and relative caudate HTT mRNA knockdown (KD), across VG doses (VG/animal, or VG) and expressed as a percentage of vehicle (veh), corresponding standard deviation (stdev) values, as well as P-values generated by one-way ANOVA with Tukey's multiple comparisons, are presented in Table 138. Note that each group (grp) of animals listed in Table 138 consisted of 3 animals per group.

TABLE 138
HTT mRNA levels in the NHP caudate after bilateral intraputaminal and intrathalamic infusion of AAV1-VOYHT1

| | | Relative | Relative | P value by | P value by | P value by | P value by | P value by |
|---|---|---|---|---|---|---|---|---|
| | Total | remaining | HTT | one-way | one-way | one-way | one-way | one-way |
| Group | dose | mRNA protein | mRNA KD | ANOVA | ANOVA | ANOVA | ANOVA | ANOVA |
| (Grp) | (VG) | (% veh ± stdev) | (% veh ± stdev) | (vs Grp A1) | (vs Grp A2) | (vs Grp A3) | (vs Grp A4) | (vs Grp A5) |
| Grp A6 | 0.00 | 100 ± 2 | 0 ± 2 | 0.9984 | 0.9861 | 0.6234 | 0.9997 | 0.9984 |
| Grp A5 | 1.35e11 | 96 ± 10 | 4 ± 10 | 0.9638 | 0.9999 | 0.8348 | 0.9811 | — |
| Grp A4 | 4.50e11 | 103 ± 7 | −3 ± 7 | >0.9999 | 0.9349 | 0.4644 | — | — |
| Grp A1 | 6.75e11 | 104 ± 3 | −4 ± 3 | — | 0.8993 | 0.4062 | — | — |
| Grp A2 | 1.35e12 | 93 ± 8 | 7 ± 8 | — | — | 0.9252 | — | — |
| Grp A3 | 2.16e12 | 83 ± 29 | 17 ± 29 | — | — | — | — | — |

On average, there was 17%, 7%, −4%, −3% and 4% HTT mRNA KD for group A3 (2.16×10¹² VG per animal), group A2 (1.35×10¹² VG per animal), group A1 (6.75×10¹¹ VG per animal), group A4 (4.50×10¹¹ VG per animal), and group A5 (1.35×10¹¹ VG per animal), respectively, relative to the vehicle group (group A6).

vii. Thalamus HTT Protein and MRNA Levels

To evaluate HTT protein and mRNA lowering in the thalamus, 4 punches were collected from the thalamus from each animal (2 per hemisphere), resulting in a total of 12 punches per group. Aliquots of pulverized thalamus punch powder were used for HTT and beta-actin protein analysis by LC-MS/MS. HTT protein levels were normalized to levels of beta-actin (internal control), and then further normalized to the vehicle group. The average relative remaining thalamus HTT protein and relative thalamus HTT protein knockdown (KD) across total VG doses (VG/animal, or VG) and expressed as a percentage of vehicle (veh), corresponding standard deviation (stdev) values, as well as P-values generated by one-way ANOVA with Tukey's multiple comparisons, are presented in Table 139, Note that each group (grp) of animals listed in Table 139 consisted of 3 animals per group.

TABLE 139
Dose-dependent HTT protein lowering in the NHP thalamus after bilateral
intraputaminal and intrathalamic infusion of AAV1-VOYHT1

| | | Relative | Relative | P value by | P value by | P value by | P value by | P value by |
|---|---|---|---|---|---|---|---|---|
| | Total | remaining | HTT | one-way | one-way | one-way | one-way | one-way |
| Group | dose | HTT protein | protein KD | ANOVA | ANOVA | ANOVA | ANOVA | ANOVA |
| (Grp) | (VG) | (% veh ± stdev) | (% veh ± stdev) | (vs Grp A1) | (vs Grp A2) | (vs Grp A3) | (vs Grp A4) | (vs Grp A5) |
| Grp A6 | 0.00 | 100 ± 12 | 0 ± 12 | 0.7683 | 0.2119 | 0.0247 | 0.9982 | 0.3459 |
| Grp A5 | 1.35e11 | 74 ± 13 | 26 ± 13 | 0.9654 | 0.9991 | 0.5714 | 0.556 | — |
| Grp A4 | 4.50e11 | 95 ± 10 | 5 ± 10 | 0.9345 | 0.3709 | 0.0488 | — | — |
| Grp A1 | 6.75e11 | 84 ± 27 | 16 ± 27 | — | 0.8584 | 0.2119 | — | — |
| Grp A2 | 1.35e12 | 69 ± 12 | 31 ± 12 | — | — | 0.7683 | — | — |
| Grp A3 | 2.16e12 | 53 ± 9 | 47 ± 9 | — | — | — | — | — |

Dose-dependent HTT protein reduction was achieved in the thalamus after bilateral combined intraputaminal and intrathalamic infusion of different total doses of A,AV1-VOYHT1. Specifically, on average, there was 47%, 31%, 16%, 5% and 26% HTT protein KD for group A3 (2.16×10¹² VG per animal), group A2 (1.35×10¹² VG per animal), group A1 (6.75×10¹¹ VG per animal), group A4 (4.50×10¹¹ VG per animal) and group A5 (1.35×10¹¹ VG per animal), respectively, relative to the vehicle group (group A6). The HTT protein KD in the thalamus was statistically significant (P<0.05 by one-way ANOVA with Tukey's multiple comparisons test) at the highest dose of 2.16×10¹² VG/animal (group A3) relative to vehicle (group A6), as well as relative to the 2nd lowest dose of 4.50×10¹¹ VG per animal (group A4).

NHP HTT mRNA levels in the thalamus were also measured by the bDNA assay, as described in Example 1 (above), in the same thalamus punches used for HTT protein quantitation, using a specific probe set against rhesus HTT, TBP. AARS and XPNPEP1. HTT mRNA levels were normalized to the geometric mean of the three housekeeping genes TBP, AARS and XPNPEP1, and then further normalized to the vehicle group. The average relative remaining thalamus HTT mRNA and relative thalamus HTT mRNA knockdown (KD), across VG doses (VG/animal, or VG) and expressed as a percentage of vehicle (veh), corresponding standard deviation (stdev) values, as well as P-values generated by one-way ANOVA with Tukey's multiple comparisons, are presented in Table 140. Note that each group (grp) of animals listed in Table 140 consisted of 3 animals per group.

TABLE 140
HTT mRNA lowering in the NHP thalamus after bilateral intraputaminal and intrathalamic infusion of AAV1-VOYHT1

| | | Relative | Relative | P value by | P value by | P value by | P value by | P value by |
|---|---|---|---|---|---|---|---|---|
| | Total | remaining | HTT | one-way | one-way | one-way | one-way | one-way |
| Group | dose | mRNA protein | mRNA KD | ANOVA | ANOVA | ANOVA | ANOVA | ANOVA |
| (Grp) | (VG) | (% veh ± stdev) | (% veh ± stdev) | (vs Grp A1) | (vs Grp A2) | (vs Grp A3) | (vs Grp A4) | (vs Grp A5) |
| Grp A6 | 0.00 | 100 ± 10 | 0 ± 10 | 0.0062 | 0.0015 | 0.0003 | 0.0249 | 0.0171 |
| Grp A5 | 1.35e11 | 55 ± 19 | 45 ± 19 | 0.988 | 0.674 | 0.162 | >0.9999 | — |
| Grp A4 | 4.50e11 | 57 ± 16 | 43 ± 16 | 0.9555 | 0.5482 | 0.1147 | — | — |
| Grp A1 | 6.75e11 | 48 ± 21 | 52 ± 21 | — | 0.9439 | 0.3849 | — | — |
| Grp A2 | 1.35e12 | 38 ± 4 | 62 ± 4 | — | — | 0.8558 | — | — |
| Grp A3 | 2.16e12 | 25 ± 3 | 75 ± 3 | — | — | — | — | — |

Significant thalamic HTT mRNA reduction was achieved in the thalamus after bilateral intraputaminal and intrathalamic infusion of different total doses of AAV1-VOYHT1. Specifically, on average, there was 75%, 62%, 52%, 43% and 45% HTT mRNA KD for group A3 (2.16×10¹² VG per animal), group A2 (1.35×10¹² VG per animal), group A1 (6.75×10¹¹ VG per animal), group M (4.50×10¹¹ VG per animal), and group A5 (1.35×10¹¹ VG per animal), respectively, relative to the vehicle group (group A6). The HTT mRNA KD in the thalamus was statistically significant (P<0.05 by one-way ANOVA with Tukey's multiple comparisons test) at all doses of AAV1-VOYHT1 versus vehicle. There was a trend (P<0.2 by one-way ANOVA with Tukey's multiple comparisons test) towards dose-dependence of HTT mRNA KD with the highest dose group versus the two lowest dose groups, although there was no statistically significant difference in thalamus mRNA KD between different doses of AAV1-VOYHT1 (P>0.05 by one-way ANOVA with Tukey's multiple comparisons test).

The effect of vector volume on HTT mRNA lowering in the thalamus was then evaluated at the highest vector concentration (2.7×10¹² vg/mL) for groups A1 (putamen/50 μL, thalamus/75 μL), A2 (putamen/100 μL, thalamus/150 μL), and A3 (putamen/150 μL thalamus/250 μL). The average relative remaining thalamus HTT mRNA and relative thalamus HTT mRNA knockdown (KD) across infusion volumes and expressed as a percentage of vehicle, corresponding standard deviation (stdev) values, as well as P-values generated by one-way ANOVA with Tukey's multiple comparisons, are presented in Table 141. Note that each group (grp) of animals listed in Table 141 consisted of 3 animals per group.

TABLE 141
HTT mRNA levels in the NHP thalamus after bilateral intraputaminal and intrathalamic
infusion of different volumes of AAV1-VOYHT1 at the same concentration

| | | Relative | Relative | P value by | P value by | P value by |
|---|---|---|---|---|---|---|
| | Total | remaining | HTT | one-way | one-way | one-way |
| Group | dose | HTT mRNA | mRNA KD | ANOVA | ANOVA | ANOVA |
| (Grp) | (VG) | (% veh ± stdev) | (% veh ± stdev) | (vs Grp A1) | (vs Grp A2) | (vs Grp A3) |
| Grp A6 | 0.00 | 100 ± 10 | 0 ± 10 | 0.0029 | 0.0009 | 0.0003 |
| Grp A1 | 6.75e11 | 48 ± 21 | 52 ± 21 | — | 0.7367 | 0.1618 |
| Grp A2 | 1.35e12 | 38 ± 4 | 62 ± 4 | — | — | 0.569 |
| Grp A3 | 2.16e12 | 25 ± 3 | 75 ± 3 | — | — | — |

Average HTT mRNA KDs of 75%, 62% and 52% were achieved in the thalamus for group A3 (putamen/150 μL, thalamus/250 μL), group A2 (putamen/100 μL, thalamus/150 μL) and group A1 (putamen/50 μL, thalamus/75 μL), respectively, that were statistically significant (P<0.01 by one-way ANOVA with Tukey's multiple comparisons test) versus vehicle (group A6). There was no statistical difference in HTT mRNA KD across different volumes of AAV1-VOYHT1 treatment at the highest vector concentration (P>0.05 by one-way ANOVA with Tukey's multiple comparisons test).

The effect of vector concentration on HTT mRNA KD in the thalamus of NHP after bilateral intraputaminal and intrathalamic administration of AAV1-VOYHT1 at the same dosing volume (putamen/100 μL, thalamus/150 μL) was also evaluated for group A5 (2.7×10¹¹ vg/mL), group A4 (9.0×10¹¹ vg/mL) and group A2 (2.7×10¹² vg/mL) versus the vehicle control group (group A6). The average relative remaining thalamus HTT mRNA and relative thalamus HTT mRNA knockdown (KD) across vector concentrations and expressed as a percentage of vehicle (veh), corresponding standard deviation (stdev) values, as well as P-values generated by one-way ANOVA with Tukey's multiple comparisons, are presented in Table 142. Note that each group (grp) of animals listed in in the below Table 142 consisted of 3 animals per group.

TABLE 142
HTT mRNA levels in the NHP thalamus after bilateral intraputaminal and intrathalamic
infusion of different concentrations of AAV1-VOYHT1 at the same volume

| | | Relative | Relative | P value by | P value by | P value by |
|---|---|---|---|---|---|---|
| | Total | remaining | HTT | one-way | one-way | one-way |
| Group | dose | mRNA protein | mRNA KD | ANOVA | ANOVA | ANOVA |
| (Grp) | (VG) | (% veh ± stdev) | (% veh ± stdev) | (vs Grp A5) | (vs Grp A4) | (vs Grp A2) |
| Grp A6 | 0.00 | 100 ± 10 | 0 ± 10 | 0.0134 | 0.0182 | 0.002 |
| Grp A5 | 1.35e11 | 55 ± 19 | 45 ± 19 | — | 0.9954 | 0.4507 |
| Grp A4 | 4.50e11 | 57 ± 16 | 43 ± 16 | — | — | 0.3452 |
| Grp A2 | 1.4e12 | 38 ± 4 | 62 ± 4 | — | — | — |

These results show that at the same dosing volume (putamen/100 μL, thalamus/150 μL), an average of 62%, 43% and 45% HTT mRNA KD was achieved in the thalamus for group A2 (2.7×10¹² vg/mL), group A4 (9.0×10¹¹ vg/mL) and group A5 (2.7×10¹¹ vg/mL), respectively. Statistically significant HTT mRNA KDs were achieved at all concentrations of AAN1-VOYHT1 (2.7×10¹² vg/mL for group A2, 9.0×10¹¹ vg/mL, for group A4, and 2.7×10¹¹ vg/mL, for group A5) versus vehicle (P<0.05 by one-way ANOVA with Tukey's multiple comparisons test). There was no statistical significance in HTT mRNA KD across different concentrations of AAV1-VOYET1 administered with the same volume (P>0.05 by one-way ANOVA with Tukey's multiple comparison test),

viii. Relationship Between Relative HTT Protein and Relative mRNA Levels

The relationship between relative HTT protein levels and relative HTT mRNA levels in all samples from putamen, caudate and thalamus from all AAV1-VOYHT1 treatment groups was assessed using a Pearson correlation method. Relative HTT protein levels as measured by the LC-MS/MS assay correlated with relative HTT mRNA levels as measured by the bDNA assay (Pearson r=0.7863, P<0.001).

Together, this study demonstrates that at 5 weeks after bilateral combined intraputaminal and intrathalamic administration of AAV1-VOYHT1, there was significant and dose-dependent HTT protein KD in the putamen. HTT protein KD in the putamen was maximal at a dose of 1.35×10¹² VG per animal, whereas in the thalamus, the highest dose resulted in significant HTT protein KD. HTT protein KD was less apparent than HTT mRNA KD in both putamen and thalamus at all dose levels. There was no significant HTT protein KD observed in the caudate at all doses tested in this study. Remaining HTT protein levels after AAV1-VOYHT1 administration, relative to vehicle, correlated with relative remaining HTT mRNA levels for all samples from putamen, caudate and thalamus.

### Example 13

### HTT Protein Evaluation in NHP Putamen, Caudate, and Thalamus after Intraputaminal and Intrathalamic Infusion of VY-HTT01 with Different Doses

i. Study Design

As an extension of Example 3 (Dose Optimization Study III), described in detail above, the primary goal of this study was to measure HTT protein levels in tissue punches from the putamen, caudate and thalamus 5 weeks following bilateral infusion, either intrathalamic-only or combined intraputaminal and intrathalamic, of different doses of AAV1-VOYHT1, in non-human primate (NHP) rhesus macaque (Macaca mulatta). As a secondary goal, HTT mRNA levels were measured in the same tissue samples in order to assess the potential relationship between relative HTT protein and relative HTT mRNA levels from the same tissue. General aspects of study implementation, including animal care/groups, test article preparation, and dosing schedules, are the same as described in Example 3 (above). The study design summarized in Table 70 similarly reflects that of the present study, apart from the omission of group C la here. Group C1b only (dosed with refined surgical procedures, as noted above) served as the control group for the treatment groups in this study.

ii. Tissue Sample Collection and Preparation

Approximately twenty-two coronal brain slices (3 mm thick) were collected for each animal using a monkey brain matrix. Brain slices were then fresh frozen and stored at −60° C. for potential pharmacology analysis or were fixed for histopathology studies.

Procedures for brain tissue sample collection for subsequent quantitation of HTT protein were similar to those described in Example 7 (above). Briefly, frozen brain slices were warmed at room temperature (for ˜1 min), and then tissue samples were rapidly collected using a 2 mm biopsy punch outfitted with a plunger, and immediately transferred to a pre-cooled collection tube on dry ice. All tissue punches were maintained on dry ice during collection and then stored at −80° C. until tissue analysis.

Procedures for tissue sample preparation for subsequent quantitation of HTT protein were as described in Example 7 (above). Briefly, brain tissue punches were weighed, pulverized using a tissue impactor, transferred into pre-chilled collection bottle, and stored on dry ice. The pulverized dry tissue powder was aliquoted into two parts: one quarter of the tissue powder was transferred to a new pre-chilled tube for HTT mRNA measurement by the branched DNA (nDNA) assay, and the remaining three-quarters of tissue powder was transferred into another pre-chilled tube for HTT protein quantification by LC-MS/MS.

iii. Branched DNA Assay for Quantitation of HTT mRNA.

Methods for quantitation of HTT mRNA by branched DNA (bDNA) assay were as described above in Example 1 (above).

iv. LC-MS/MS for Quantitation of HTT Protein

Methods for quantitation of HTT protein by UPLC-MS/MS (Ultra performance liquid chromatography-mass spectrometry and liquid chromatography-tandem mass spectrometry), or LC-MS/MS, were as described above in Example 7 (above). All samples were run at the same time for HTT protein quantification by LC-MS/MS.

v. Putamen HTT Protein and mRNA Levels

To evaluate HTT protein and mRNA levels in the putamen. 4 tissue punches were collected from the putamen in the right hemisphere, resulting in a total of 12 punches group. As described above, aliquoted pulverized putamen tissue punch powders were used for HTT and actin protein analysis by LC-MS/MS. HTT protein levels were normalized to the levels of beta-actin, and then further normalized to the vehicle (veh) group, The average relative remaining putamen HTT protein and relative putamen HTT protein knockdown (KD) across total VG doses (VG/animal, or VG) and expressed as a percentage of vehicle (veh), corresponding standard deviation (stdev) values, as well as P-values generated by one-way ANOVA with Tukey's multiple comparisons, are presented in Table 143. Note that each group (grp) of animals listed in Table 143 consisted of 3 animals per group.

TABLE 143
HTT protein lowering in NHP putamen

| | | Relative | Relative | P value by | P value by | P value by |
|---|---|---|---|---|---|---|
| | Total | remaining | HTT | one-way | one-way | one-way |
| Group | dose | HTT protein | protein KD | ANOVA | ANOVA | ANOVA |
| (Grp) | (VG) | (% veh ± stdev) | (% veh ± stdev) | (vs Grp C2) | (vs Grp C3) | (vs Grp C4) |
| Grp C1b | 0.0 | 100 ± 5 | 0 ± 5 | 0.9001 | <0.0001 | <0.0001 |
| Grp C2 | 1.11e13 | 96 ± 8 | 4 ± 8 | — | <0.0001 | <0.0001 |
| Grp C3 | 8.00e12 | 35 ± 8 | 65 ± 8 | — | — | 0.9447 |
| Grp C4 | 1.78e13 | 39 ± 9 | 61 ± 9 | — | — | — |

Significant HTT protein reduction was achieved in the putamen after bilateral intraputaminal and intrathalamic infusion of different doses of AAV l-VOYHT1. Specifically, on average, there was 65% and 61% HTT protein KD at combined intraputaminal and intrathalamic doses of 8.00×10¹² VG per animal (group C3) and 1.78×10¹³ VG per animal (group C4), respectively, relative to the vehicle group (group C1b), which did not exhibit HTT protein KD (P<0.0001 by one-way ANOVA with Tukey's multiple comparisons test). HTT protein KD after combined intraputaminal and intrathalamic doses of 8,00×10¹² and 1.78×10¹³ VG per animal was also greater (P<0.0001 by one-way ANOVA Tukey's multiple comparisons test) than after intrathalamic dosing only (group 2C, 1.11×10¹³ VG per animal). There was no significant difference between HTT protein KD at a bilateral intraputaminal and intrathalamic dose of 8.00×10¹² versus 1,78×10¹³ VG per animal. In addition, no significant HTT protein KD was observed after intrathalamic dosing only (group C2) relative to vehicle (group C1b) (P>0.05 by one-way ANOVA with Tukey's multiple comparisons test).

HTT mRNA levels in the putamen were also measured by the hDNA assay, as described in Example 1, in the same putamen punches used for HTT protein quantitation, using the specific probe set against rhesus HTT, TBP, AARS and XPNPEP1. HTT mRNA levels were normalized to the geometric mean of the three housekeeping genes TBP, AARS and XPNPEP1 and then further normalized to the vehicle group. The average relative remaining putamen HTT mRNA and relative putamen HTT mRNA knockdown (KD) across total VG doses (VG/animal, or VG) and expressed as a percentage of vehicle (veh), corresponding standard deviation (stdev) values, as well as P-values generated by one-way ANOVA with Tukey's multiple comparisons, are presented in Table 144. Note that each group (grp) of animals listed in Table 144 consisted of 3 animals per group.

TABLE 144
HTT mRNA lowering in NHP putamen

| | | Relative | Relative | P value by | P value by | P value by |
|---|---|---|---|---|---|---|
| | Total | remaining | HTT | one-way | one-way | one-way |
| Group | dose | HTT mRNA | mRNA KD | ANOVA | ANOVA | ANOVA |
| (Grp) | (VG) | (% veh ± stdev) | (% veh ± stdev) | (vs Grp C2) | (vs Grp C3) | (vs Grp C4) |
| Grp C1b | 0.0 | 100 ± 5 | 0 ± 5 | 0.0109 | <0.0001 | <0.0001 |
| Grp C2 | 1.11e13 | 83 ± 7 | 17 ± 7 | — | <0.0001 | <0.0001 |
| Grp C3 | 8.00e12 | 34 ± 2 | 66 ± 2 | — | — | 0.213 |
| Grp C4 | 1.78e13 | 42 ± 3 | 58 ± 3 | — | — | — |

Significant HTT mRNA reduction was achieved in the putamen after bilateral intrathalamic or bilateral combined intraputaminal and intrathalamic infusion of different total doses of AAV1-VOYHT1. Specifically, on average, there was 17% 66% and 58% HTT mRNA KD for group C2 (thalamus only: 1.11×10¹³ VG per animal), group C3 (combined intraputaminal and intrathalamic dosing: 8.00×10¹² VG per animal) and group C4 (combined intraputaminal and intrathalamic dosing: 1.78×10¹³ VG per animal), respectively, relative to the vehicle group (group C1b) that was statistically significant (P<0.05 by one-way ANOVA with Tukey's multiple comparisons test). HTT mRNA KD after combined intraputaminal and intrathalamic doses of 8.00×10¹² VG per animal and 1.78×10¹³ VG per animal was also greater (P<0.0001 by one-way ANOVA with Tukey's multiple comparisons test) than after intrathalamic dosing only (group C2, 1.11×10¹³ VG per animal). There was no significant difference between HTT mRNA KD at a bilateral intraputaminal and intrathalamic dose of 8.00×10¹² versus 1.78×10¹³ VG per animal (P>0.05 by one-way ANOVA with Tukey's multiple comparisons test)

vi. Caudate HTT Protein and mRNA Levels

To evaluate HTT protein and mRNA lowering in the caudate, 4 punches were collected from each animal (2 per hemisphere) for HTT and actin protein analysis by LC-MS/MS, resulting in a total of 12 punches per group. HTT protein levels were normalized to level of actin (internal control), and then further normalized to the vehicle group. The average relative remaining caudate HTT protein and relative caudate HTT protein knockdown (KD) across total VG doses (VG/animal, or VG) and expressed as a percentage of vehicle (veh), corresponding standard deviation (stdev) values, as well as P-values generated by one-way ANOVA with Tukey's multiple comparisons, are presented in Table 145. Note that each group (grp) of animals listed in Table 145 consisted of 3 animals per group.

TABLE 145
HTT protein lowering in NHP caudate

| | | Relative | Relative | P value by | P value by | P value by |
|---|---|---|---|---|---|---|
| | Total | remaining | HTT | one-way | one-way | one-way |
| Group | dose | HTT protein | protein KD | ANOVA | ANOVA | ANOVA |
| (Grp) | (VG) | (% veh ± stdev) | (% veh ± stdev) | (vs Grp C2) | (vs Grp C3) | (vs Grp C4) |
| Grp C1b | 0.0 | 100 ± 6 | 0 ± 6 | 0.015 | 0.0011 | 0.0006 |
| Grp C2 | 1.11e13 | 74 ± 7 | 26 ± 7 | — | 0.2025 | 0.1024 |
| Grp C3 | 8.00e12 | 60 ± 9 | 40 ± 9 | — | — | 0.9584 |
| Grp C4 | 1.78e13 | 56 ± 9 | 44 ± 9 | — | — | — |

Significant HTT protein reduction was achieved in the caudate after bilateral intrathalamic or bilateral combined intraputaminal and intrathalamic infusion of different total doses of AAV1-VOYHT1. Specifically, on average, there was 26%, 40% and 44% HTT protein KD for group C2 (thalamus dosing only, 1.11×10¹³ VG per animal), group C3 (combined intraputaminal and intrathalamic dosing, 8.00×10¹² VG per animal) and group C4 (combined intraputaminal and intrathalamic dosing, 1.78×10¹³ VG per animal), respectively, relative to the vehicle group (group C1b) that was statistically significant (P<0.05 by one-way ANOVA with Tukey's multiple comparisons test). There was no difference in HTT protein KD across all AAV1-VOYHT1 treatment groups (P>0.05 by one-way ANOVA with Tukey's multiple comparisons test)

NHP caudate HTT mRNA levels were also measured by the bDNA assay in the same caudate punches used for HTT protein quantitation, using the specific probe set against rhesus HTT, TBP, AARS and XPNPEP1. HTT mRNA levels were normalized to the geometric mean of the three housekeeping genes TBP, AARS and XPNPEP1, and then further normalized to the vehicle group. The average relative remaining caudate HTT mRNA and relative caudate HTT mRNA knockdown (KD) across total VG doses (VG/animal, or VG) and expressed as a percentage of vehicle (veh), corresponding standard deviation (stdev) values, as well as P-values generated by one-way ANOVA with Tukey's multiple comparisons, are presented in Table 146. Note that each group (grp) of animals listed in Table 146 consisted of 3 animals per group.

TABLE 146
HTT mRNA lowering in NHP caudate

| | | Relative | Relative | P value by | P value by | P value by |
|---|---|---|---|---|---|---|
| | Total | remaining | HTT | one-way | one-way | one-way |
| Group | dose | HTT mRNA | mRNA KD | ANOVA | ANOVA | ANOVA |
| (Grp) | (VG) | (% veh ± stdev) | (% veh ± stdev) | (vs Grp C2) | (vs Grp C3) | (vs Grp C4) |
| Grp C1b | 0.0 | 100 ± 8 | 0 ± 8 | 0.027 | 0.0024 | 0.0018 |
| Grp C2 | 1.11e13 | 63 ± 13 | 37 ± 13 | — | 0.296 | 0.2152 |
| Grp C3 | 8.00e12 | 43 ± 12 | 57 ± 12 | — | — | 0.9944 |
| Grp C4 | 1.78e13 | 41 ± 16 | 59 ± 16 | — | — | — |

Significant HTT mRNA reduction was observed in the caudate after bilateral intrathalamic or bilateral combined intraputaminal and intrathalamic infusion of different doses of AAV1-VOYHT1. Specifically, on average, there was 37%, 57% and 59% HTT mRNA KD for group C2 (thalamus dosing only, 1.11×10¹³ VG per animal), group C3 (combined intraputaminal and intrathalamic infusion, 8.00×10¹² VG per animal) and group C 4 (combined intraputaminal and intrathalamic infusion, 1.78×10¹³ VG per animal), respectively, relative to the vehicle group (group C1b) that was statistically significant (P<0.05 by one-way ANOVA with Tukey's multiple comparisons test). There was no significant difference in caudate HTT mRNA KD across AAV1-VOYHT1 treatment groups (P>0.05 by one-way ANOVA with Tukey's multiple comparisons test)

vii. Thalamus HTT Protein and mRNA Levels

To evaluate HTT protein and mRNA lowering in the thalamus, 4 punches were collected from each animal (2 per hemisphere) for HTT and actin protein analysis by LC-MS/MS, resulting in a total of 12 punches per group (3 animals per group), Thalamus HTT protein levels were normalized to the levels of actin (internal control), and then fluffier normalized to the vehicle group. The average relative remaining thalamus HTT protein and relative thalamus HTT protein knockdown (KD) across total VG doses (VG/animal, or VG) and expressed as a percentage of vehicle (veh), corresponding standard deviation (stdev) values, as well as P-values generated by one-way ANOVA with Tukey's multiple comparisons, are presented in Table 147. Note that each group (grp) of animals listed in Table 147 consisted of 3 animals per group.

TABLE 147
HTT protein lowering in NHP thalamus

| | | Relative | Relative | P value by | P value by | P value by |
|---|---|---|---|---|---|---|
| | Total | remaining | HTT | one-way | one-way | one-way |
| Group | dose | HTT protein | protein KD | ANOVA | ANOVA | ANOVA |
| (Grp) | (VG) | (% veh ± stdev) | (% veh ± stdev) | (vs Grp C2) | (vs Grp C3) | (vs Grp C4) |
| Grp C1b | 0.0 | 100 ± 1 | 0 ± 1 | <0.0001 | <0.0001 | <0.0001 |
| Grp C2 | 1.11e13 | 46 ± 4 | 54 ± 4 | — | 0.4428 | 0.0072 |
| Grp C3 | 8.00e12 | 52 ± 9 | 48 ± 9 | — | — | 0.0012 |
| Grp C4 | 1.78e13 | 26 ± 3 | 74 ± 3 | — | — | — |

Dose-dependent HTT protein reduction was achieved in the thalamus after bilateral combined intraputaminal and intrathalamic infusion of different total doses of AAV1-VOYHT1. Specifically, on average, there was 54%, 48% and 74% HTT protein KD for group C2 (thalamus dosing only, 1.11×10¹³ VG per animal), group C3 (combined intraputaminal and intrathalamic infusion 8.00×10¹² VG per animal) and group C4 (combined intraputaminal and intrathalamic infusion, 1.78×10¹³ VG per animal), respectively, relative to the vehicle group (group C1b that was statistically significant (P<0.0001 by one-way ANOVA with Tukey's multiple comparisons test). HTT protein KD in the thalamus was greater (P<0.05 by one-way ANOVA with Tukey's multiple comparisons test) after combined intraputaminal and intrathalamic administration of AAV1-VOYHT1 at a dose of 1.78×10¹³ VG per animal (group C4) versus thalamus administration only at a dose of 1.11×10¹³ VG per animal (group C2). HTT protein KD in the thalamus was dose-dependent, with significantly greater KD (P<0.01 by one-way ANOVA with Tukey's multiple comparisons test) after combined intraputaminal and intrathalamic administration of AAV1-VOYHT1 at a dose of 1.78×10¹³ (group C4) versus 8.00×10¹² (group C3) VG per animal.

NHP thalamic HTT mRNA levels were also measured by bDNA assay in the same thalamus punches used for HTT protein quantitation, using the specific probe set against rhesus HTT, TBP, AARS and XPNPEP1. Thalamic HTT mRNA levels were normalized to the geometric mean of the three housekeeping genes TBP, AARS and XPNPEP1, and then further normalized to the vehicle group. The average relative remaining thalamus HTT mRNA and relative thalamus HTT mRNA knockdown (KD) across total VG doses (VG/animal, or VG) and expressed as a percentage of vehicle (veh), corresponding standard deviation (stdev) values, as well as P-values generated by one-way ANOVA with Tukey's multiple comparisons, are presented in Table 148. Note that each group (grp) of animals listed in Table 148 consisted of 3 animals per group.

TABLE 148
HTT mRNA lowering in NHP thalamus

| | | Relative | Relative | P value by | P value by | P value by |
|---|---|---|---|---|---|---|
| | Total | remaining | HTT | one-way | one-way | one-way |
| Group | dose | HTT mRNA | mRNA KD | ANOVA | ANOVA | ANOVA |
| (Grp) | (VG) | (% veh ± stdev) | (% veh ± stdev) | (vs Grp C2) | (vs Grp C3) | (vs Grp C4) |
| Grp C1b | 0.0 | 100 ± 3 | 0 ± 3 | <0.0001 | <0.0001 | <0.0001 |
| Grp C2 | 1.11e13 | 29 ± 3 | 71 ± 3 | — | 0.9988 | 0.8485 |
| Grp C3 | 8.00e12 | 29 ± 6 | 71 ± 6 | — | — | 0.7768 |
| Grp C4 | 1.78e13 | 32 ± 7 | 68 ± 7 | — | — | — |

Significant thalamic HTT mRNA reduction was achieved in the thalamus after bilateral intrathalamic or bilateral combined intraputaminal and intrathalamic infusion of different total doses of AAV1-VOYHT1. Specifically, on average, 71%, 71% and 68% HTT mRNA KD for group C2 (thalamus dosing only, 1.11×10¹³ VG per animal), group C3 (combined intraputaminal and intrathalamic dosing, 8.00×10¹² VG per animal), and group C4 (combined intraputaminal and intrathalamic dosing, 1.78×10¹³ VG per animal), respectively, relative to vehicle (group C1b) that was statistically significant (P<0.0001 by one-way ANOVA with Tukey's multiple comparisons test). There was no statistically significant difference in thalamus HTT mRNA KD across AAV1-VOYHT1 treatment groups (P>0.05 by one-way ANOVA with Tukey's multiple comparisons test).

viii. Relationship Between Relative HTT Protein and Relative mRNA Levels

The relationship between relative HTT protein levels and relative HTT mRNA levels in all samples from putamen, caudate and thalamus from group C2 (1.11×10¹³ VG per animal), group C3 (8.00×10¹² VG per animal), group C4 (1.78×10¹³ VG per animal), and the vehicle group (group C1b was assessed using a Pearson correlation method. Relative HTT protein levels as measured by the LC-MS/MS assay correlated with relative HTT mRNA levels as measured by the bDNA assay (Pearson r=0.9334, P<0.0001).

Together, this study demonstrated that at 5 weeks after bilateral combined intraputaminal and intrathalamic administration of VY-AAV1-VOYHT1, there was significant HTT protein KD in the putamen, caudate and thalamus. HTT protein KD in the putamen and caudate was maximal at 8.00×10¹² VG per animal tested in this study. Combined intrathalamic and intraputaminal infusion of VY-HTT01. resulted in greater HTT protein KD in the putamen and thalamus than administration via thalamus only. In the caudate, HTT protein KD was similar with combined intrathalamic and intraputaminal infusion of VY-HTT01 vs intrathalamic dosing only. Relative remaining HTT protein levels correlated with relative remaining HTT mRNA levels for all samples tested from putamen, caudate and thalamus.

### Example 14

### VY-HTT01-Mediated HTT mRNA and Protein Knockdown in FRhK-4 Cells

This study was designed to measure HTT mRNA and protein knockdown in FRhK-4 rhesus macaque (Macaca mulatta) kidney cells transduced with AAV1-VOYHT1 For AAV vector transduction, cells were seeded at a density of 5e6 cells/well in P150 dishes and subsequently transfected via bath (i.e., culture medium) application of AAV1-VOYHT1 at the following vector doses (expressed as multiplicity of infection, or MOI, in vg/cell): 0, 5.0e2, 1.6e3, 5.0e3, 1.6e4, 5.0e4, 1.6e5 and 5.0e5 vg/cell. After 48 hours, AAV1-VOYHT1-transduced cells were harvested and subdivided into three cell pellets for measurement of relative HTT mRNA by qRT-PCR, and for measurement of relative HTT protein by LC-MS/MS and western blot.

HTT mRNA quantification procedures were similar to those described in Example 8. Briefly, relative HTT mRNA expression was obtained by normalizing the HTT mRNA level to housekeeping gene mRNA level; this normalized HTT mRNA level was then expressed relative to the normalized HTT mRNA level in treated and/or untreated cells, as determined by qRT-PCR. The average relative remaining FRhK-4 cell HTT mRNA and relative FRhK-4 cell HTT mRNA knockdown (KD) expressed as a percentage of vehicle (veh) across AAV1-VOYHT1 doses (MOI), and as determined by qRT-PCR, are provided in Table 149.

TABLE 149
HTT mRNA knockdown by AAV1-VOYHT1 in FRhK-4 cells

| | Relative remaining |
|---|---|
| Dose | HTT mRNA |
| (MOI) | (% veh ± stdev) |
| 5.0e5 | 13.2 ± 0.0 |
| 1.6e5 | 15.1 ± 2.0 |
| 5.0e4 | 20.7 ± 7.6 |
| 1.6e4 | 40.6 ± 0.0 |
| 5.0e3 | 46.3 ± 6.8 |
| 1.6e3 | 88.0 ± 38.2 |
| 5.0e2 | 109.3 ± 3.2 |
| 0 | 100.0 ± 30.1 |

These findings demonstrate dose-dependent FRhK-4 HTT mRNA knockdown, with 87% of HTT mRNA knockdown achieved at MO1 5e5.

Procedures for HTT protein quantification by LC-MS/MS were similar to those described in Example 7. HTT protein levels were also assessed by standard western blotting methodology known and performed by a person skilled in the art. The relative remaining FRhK-4 cell HTT protein and relative FRhK-4 cell HTT protein knockdown (KD), expressed as a percentage of vehicle (veh) across AAV1-VOYHT1 doses (MOI), as determined by LC-MS and western blot approaches, are presented in Table 150. For LC-MS analyses, the concentration (cone) of HTT protein (ng/mg total protein) measured in FRhK-4 cells is also included in Table 150 for clarity and completeness.

TABLE 150
HTT protein knockdown by AAV1-VOYHT1 in FRhK-4 cells

| | LC-MS/MS | | |
|---|---|---|---|
| | Conc of HTT | | Western Blot |
| | protein in | Relative | Relative |
| | homogenate | remaining | remaining |
| Dose | (ng/mg total | HTT protein | HTT protein |
| (MOI) | protein) | (% veh) | (% veh) |
| 5.0e5 | 14.1 | 12.5 | 31.9 |
| 1.6e5 | 21.7 | 19.1 | 46.0 |
| 5.0e4 | 30.6 | 27.0 | 55.2 |
| 1.6e4 | 46.2 | 40.7 | 84.6 |
| 5.0e3 | 60.7 | 53.5 | 143.3 |
| 1.6e3 | 85.2 | 75.1 | 197.7 |
| 5.0e2 | 100.8 | 88.8 | 234.8 |
| 0 | 113.5 | 100.0 | 100.0 |

Together, HTT protein quantification analyses revealed dose-dependent knockdown of HTT protein by AAV1-VOYHT1 in FRhK-4 cells regardless of protein quantification methodology, i.e., when protein was quantified by LC-MS/MS or by western blot.

The relationship between relative remaining HTT protein and mRNA levels in FRhK-4 HTT was confirmed using a Pearson correlation analysis. When HTT protein levels were measured by LC-MS/MS, a positive linear relationship emerged between relative remaining HTT protein and mRNA levels (Pearson r=0.96, P<0.0001). Similarly, a positive linear relationship also emerged between relative HTT protein and mRNA levels (Pearson r=0.55, P<0.05) when HTT protein levels were measured by western blot. Thus, regardless of protein quantification methodology applied, higher HTT protein levels corresponded to higher HTT mRNA levels.

This study revealed dose-dependent relative HTT mRNA and protein knockdown in FRhK-4 rhesus macaque (Macaca mulatta) kidney cells transduced with AAV1-VOYHT1, with nearly 90% of HTT mRNA knockdown achieved at the highest dose (MOI 5e5 vg/cell). Protein knockdown was observed when measured by both LC-MS/MS and western blot and a strong positive relationship emerged between HTT protein and HTT mRNA levels regardless of protein quantification methodology applied.

### Equivalents and Scope

Those skilled in the art will recognize or be able to ascertain using no more than routine experimentation, many equivalents to the specific embodiments in accordance with the AAV particles described herein. The scope of the present disclosure is not intended to be limited to the above Description, but rather is as set forth in the appended claims,

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used. herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein, Any particular embodiment of the compositions of the disclosure (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the disclosure in its broader aspects.

While the present disclosure has been described at some length and with some particularity with respect to the several described embodiments, it is not intended that it should be limited to any such particulars or embodiments or any particular embodiment, but it is to he construed with references to the appended claims so as to provide the broadest possible interpretation of such claims in view of the prior art and, therefore, to effectively encompass the intended scope of the disclosure.

1. A pharmaceutical composition for use in the treatment of Huntington's Disease, said composition comprising adeno-associated virus (AAV) particles in a pharmaceutically acceptable formulation; wherein at least one of the AAV particles comprises an AAV viral genome comprising a polynucleotide sequence selected from the group consisting of SEQ ID NO: 1352-1379, 1388, and 1426-1438 or variants of the foregoing having at least 95% identity thereto; and wherein the concentration of the AAV viral genome in the pharmaceutical composition is from 1×10¹¹ to 1×10¹² VG/mL.

2. The pharmaceutical composition of claim 1, wherein the concentration of the AAV viral genome is from 1×10¹¹to 9×10¹¹ VG/mL.

3. The pharmaceutical composition of claim 1, wherein the concentration of the AAV viral genome is from 1.2×10¹¹ to 6×10¹¹ VG/mL.

4. The pharmaceutical composition of claim 1, wherein the concentration of the AAV viral genome is from 1.8×10¹¹ to 6×10¹¹ VG/mL.

5. The pharmaceutical composition of claim 1, wherein the concentration of the AAV viral genome is from 5×10¹¹ to 8×10¹¹ VG/mL.

6. The pharmaceutical composition of any one of claims 1-5, wherein the polynucleotide sequence comprises SEQ ID NO: 1352.

7. The pharmaceutical composition of any one of claims 1-6, wherein the AAV particles comprise an AAV capsid and said capsid serotype is selected from the group consisting of AAV1, AAV2, AAV2G9, AAV3, AAV3a, AAV3b, AAV3-3, AAV4, AAV4-4, AAV5, AAV6, AAV6.1, AAV6.2, AAV6.1.2, AAV7, AAV7.2, AAV8, AAV9, AAV9.11, AAV9.13, AAV9.16, AAV9.24, AAV9.45, AAV9.47, AAV9.61, AAV9.68, AAV9.84, AAV9.9, AAV10, AAV11, AAV12, AAV16.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/r11.64, AAV4-9/rh.54, AAV4-19/rh.5.5, AAV5-3/rh.57, AAV5-22/rh58, AAV7.3/hu.7, AAV 16.81hu.10-NAV16.12/hu.11, AAV29.3/bh 1, AAV29.5/bb.2, AAV106.1/hu.37, AAV114.3/hu.40, AAV127.2/hu.41, AAV127.5/hu.42, AAV128.3/hu.44, AAV130.4/hu.48, AAV 145.1/hu.53, AAV145.5/hu.54, AAV145.6/hu.55, AAV161.10/hu.60, AAV161.6/hu.61, AAV33.12/hu.17, AAV33.4/hu.15, AAV33.84/hu.16, AAV52.1hu.19, AAV52.1/hu.20, AAV58.2/hu.25, AAVA3.3, AAVA3.4, AAVA3.5, AAVA3.7, AAVC1, AAVC2, AAVC5, AAV-DJ, AAV-DJ8, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVLK03, AAVH-1/hu.1, AAVH-5.1hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1., AAVCy.5R2, AAVCy.5R3, AAVCy.5R4, AAVcy,6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39. AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74, AAVrh8R, AAVrh8R A586R mutant, AAVrh8R R533A mutant, AAAV, BAAV, caprine AAV, bovine AAV, ovine AAV, AAVhE1.1, AAVhEr1.5, AAVhER1.14, AAVhEr1.8, AAVhEr1 AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER.1.23, AAVhEr3.1, AAV2.5T AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC 11, AAV-PAEC12, AAV-2-pre-miRNA-101, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6. AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPENN AAV 10, Japanese AAV 10 serotypes, AAV CBr-7.1, AAV CBr-7.10, AAV CBr-7.2, AAV CBr-7.3, AAV CBr-7.4, AAV CBr-7.5, AAV CBr-7.7, AAV CBr-7.8, AAV CBr-B7.3, AAV CBr-B7.4, AAV CBr-E1, AAV CBr-E2, AAV CBr-E3, AAV CBr-E4,AAV CBr-E5, AAV CBr-e5,AAV CBr-E6, AAV CBr-E7, AAV CBr-E8, AAV CHt-1, AAV CHt-2, AAV CHt-3, AAV CHt-6.1, AAV CHt-6.10, AAV CHt-6.5, AAV CHt-6.6, AAV CHt-6.7, AAV CHt-6.8, AAV CHt-P1, AAV CHt-P2, AAV CHt-P5, AAV CHt-P6, AAV CHt-P8, AAV CHt-P9, AAV CKd-1, AAV CKd-10, AAV CKd-2, AAV CKd-3, AAV CKd-4, AAV CKd-6, AAV CKd-7,AAV CKd-8, AAV CKd-B1, AAV CKd-B2, AAV CKd-B3, AAV CKd-B4, AAV CKd-B5, AAV CKd-B6, AAV CKd-B7, AAV CKd-B8, AAV CKd-H1, AAV CKd-H2, AAV CKd-H3, AAV CKd-H4, AAV CKd-H5, AAV CKd-H6, AAV CKd-N3, AAV CKd-N4, AAV CKd-N9, AAV CLg-F1, AAV CLg-F2,AAV CLg-F3, AAV CLg-F4, AAV CLg-F5, AAV CLg-F6,AAV CLg-F7, AAV CLg-F8, AAV CLv-1, AAV CLv1-1, AAV Clv1-10, AAV CLv1-2, AAV CLv-12, AAV CLv1-3, AAV CLv-13, AAV CLv1-4, AAV CLv1-7, AAV Clv1-8, AAV CLv-1-9, AAV CLv-2, AAV CLv-3, AAV CLv-4, AAV CLv-6, AAV CLv-8, AAV CLv-D1, AAV CLv-D2, AAV CLv-D3, AAV CLv-D4, AAV CLv-D5, AAV CLv-D6, AAV CLv-D7, AAV CLv-D8, AAV CLv-E1, AAV CLv-K1, AAV CLv-K3, AAV CLv-K6, AAV CLv-L4, AAV CLv-L5, AAV CLv-L6, AAV CLv-M1, AAV CLv-M11, AAV CLv-M2, AAV CLv-M5, AAV CLv-M6, AAV CLv-M7, AAV CLv-M8, AAV CLv-M9, AAV CLv-R1, AAV CLv-R2, AAV CLv-R3, AAV CLv-R4, AAV CLv-R5, AAV CLv-R6, AAV CLv-R7, AAV CLv-R8, AAV CLv-R9, AAV CSp-1, AAV CSp-10, AAV CSp-11, AAV CSp-2, AAV CSp-3, AAV CSp-4, AAV CSp-6, AAV CSp-7, AAV CSp-8,AAV CSp-8.10, AAV CSp-8.2, AAV CSp-8.4, AAV CSp-8.5, AAV CSp-8.6, AAV CSp-8.7, AAV CSp-8.8, AAV CSp-8.9, AAV CSp-9, AAV.hu.48R3, AAV.VR-355, AAV3B, AAV4, AAV5, AAVF1/HSC1, AAVF11/HSC11, AAVF12/HSC12, AAVF13/HSC13, AAVF14/HSC14, AAVF15/HSC15, AAVF16/HSC16, AAVF17/HSC17, AAVF2/HSC2, AAVF3/HSC3, AAVF4/HSC4, AAVF5/HSC5, AAVF6/HSC6, AAVF7/HSC7, AAVF8/HSC8, AAVF9/HSC9, AAV-PHP.B, AAV-PHP.A, G2B-26, G2B-13, TH1.1-32, TH1.1-35, AAVPHP.B2, AAVPHP.B3, AAVPHP.N/PHP.B-DGT, AAVPHP.B-EST, AAVPHP.B-GGT, AAVPHP.B-ATP, AAVPHP.B-ATT-T, AAVPHP.B-DGT-T, AAVPHP.B-GGT-T, AAVPHP.B-SGS, AAVPHP.B-AQP, AAVPHP.B-QQP, AAVPHP.B-SNP(3), AAVPHP.B-SNP, AAVPHP.B-QGT, AAVPHP.B-NQT, AAVPHP.B-EGS, AAVPHP.B-SGN, AAVPHP.B-EGT, AAVPHP.B-DST, AAVPHP.B-DST, AAVPHRB-STP, AAVPHRB-PQP, AAVPHP.B-SQP, AAVPHP.B-QLP, AAVPHP.B-TMP, AAVPHP.B-TTP, AAVPHP.S/G2A12, AAVG2A15/G2A3, AAVG2B4, and/or AAVG2B5 and variants thereof.

8. The pharmaceutical composition of claim 7, wherein the AAV particle capsid serotype is an AAV1 serotype.

9. The pharmaceutical composition of claim 7, wherein the pharmaceutically acceptable formulation comprises:
a) one or more salts selected from sodium chloride, potassium chloride, and potassium phosphate;
b) at least one disaccharide, wherein the at least one disaccharide comprises sucrose; and
c) a buffering agent selected from Tris HCl, Tris base, sodium phosphate, potassium phosphate, histidine, boric acid, citric acid, glycine, HEPES (4-(2-hydroxyethyl)-1-piperazineethanesulfonic acid), and MOPS (3-(N-morpholino)propanesulfonic acid);
wherein said pharmaceutical composition is an aqueous solution.

10. The pharmaceutical composition of claim 9, wherein the pharmaceutically acceptable formulation comprises:
a) sodium chloride at a concentration from 85 to 110 mM;
b) potassium chloride at a concentration from 1 to 3 mM;
c) potassium phosphate at a concentration from 1 to 3 mM;
d) sucrose that is 5 to 9% by weight relative to the total volume of said formulation; and
e) a buffering agent at a concentration from 1 to 20 mM.

11. The pharmaceutical composition of claim 10, wherein the pharmaceutically acceptable formulation comprises:
a) sodium chloride at a concentration of 95 mM;
b) potassium chloride at a concentration of 1.5 mM;
c) potassium phosphate at a concentration of 1.5 mM;
d) sucrose at a concentration that is 7% by weight relative to the total volume of said formulation; and
e) the buffering agent at a concentration of 10 mM.

12. The pharmaceutical composition of any one of claim 9-11 which is buffered to a pH from 7.2 to 8.2 at 5° C.

13. The pharmaceutical composition of any one of claim 9-11, wherein the buffering agent is sodium phosphate and the formulation is buffered to a pH from 7.2 to 7.6 at 5° C.

14. The pharmaceutical composition of any one of claim 9-11, wherein the buffering agent is Tris base and is adjusted with hydrochloric acid to a pH from 7.3 to 7.7 at 5° C.

15. The pharmaceutical composition of claim 7, wherein the pharmaceutically acceptable formulation further comprises a surfactant.

16. The pharmaceutical composition of claim 15, wherein the surfactant is Poloxamer 188.

17. The pharmaceutical composition of claim 16, wherein the concentration of Poloxamer 188 is 0.001% by weight relative to the total volume of the pharmaceutically acceptable formulation.

18. The pharmaceutical composition of claim 1, claim 7 or claim 15, wherein the pharmaceutically acceptable formulation has an osmolality of 400 to 480 mOsm/kg.

19. A method of treating; Huntington's Disease in a patient in need thereof, comprising;
administering to said patient a therapeutically effective amount of the pharmaceutical composition of any one of claims 1-18.

20. The method of claim 19, wherein the pharmaceutical composition is administered by infusion into the striatum of the patient.

21. The method of claim 20, wherein the pharmaceutical composition is administered by bilateral infusion into the striatum of the patient.

22. The method of claim 19. wherein the pharmaceutical composition is administered by infusion into the putamen and thalamus of the patient.

23. The method of claim 22, wherein the pharmaceutical composition is administered by bilateral infusion into the putamen and thalamus of the patient.

24. The method of claim 22 or 23, wherein the pharmaceutical composition is administered using magnetic resonance imaging (MRI)-guided convection enhanced delivery (CED).

25. The method of any one of claims 20-21, wherein the volume of the pharmaceutical composition administered to the striatum is 15 μL/hemisphere or less.

26. The method of claim 25, wherein the volume of the pharmaceutical composition administered to the striatum is from 5-10 μL/hemisphere.

27. The method of claim 20-21, wherein the close administered to the striatum is between 2×10⁹ to 3×10¹¹ VG/hemisphere.

28. The method of any one of claims 22-24, wherein the volume of the pharmaceutical composition administered to the putamen is 1500 μL/hemisphere or less.

29. The method of claim 28, wherein the volume of the pharmaceutical composition administered to the putamen is from 100-1500 μL/hemisphere.

30. The method of claim 22-24, wherein the dose administered to the putamen is between 1×10¹⁰ to 4×10¹³ VG/hemisphere.

31. The method of any one of claims 22-24, wherein the volume of the pharmaceutical composition administered to the thalamus is 2500 μL/hemisphere or less.

32. The method of claim 31, wherein the volume of the pharmaceutical composition administered to the thalamus is from 150-2500 μL/hemisphere.

33. The method of claims 22-24, wherein the dose administered to the thalamus is between 4×10¹⁰ to 6.8×10¹³ VG/hemisphere.

34. The method of any one of claims 20-24, wherein the total dose administered to the patient is between 8×10⁹ to 2×10¹⁴ VG.

35. A method of inhibiting or suppressing the expression of a Huntingtin (HTT) gene product (RNA or protein) in a tissue selected from the group consisting of the striatum, putamen, caudate, thalamus, cerebral cortex, primary motor cortex, primary somatosensory cortex, temporal cortex and combinations thereof of a patient comprising administering a therapeutically effective amount of the pharmaceutical composition of any one of claims 1-18.

36. The method of claim 35, wherein the expression of the HTT gene product (RNA or protein is reduced by at least 30%.

37. The method of claim 35, wherein the expression of the HTT gene product (RNA or protein) is reduced by 40-70%.

38. The method of claim 35, wherein the expression of the HTT gene product (RNA or protein) is reduced by 50-80%.

39. The method of claim 35, wherein the expression of the HTT gene product (RNA or protein) is inhibited or suppressed in the putamen and wherein the inhibition or suppression is measured in one or more medium spiny neurons in the putamen.

40. The method of claim 35. wherein the expression of the HTT gene product (RNA or protein) is inhibited or suppressed in the putamen and wherein the inhibition or suppression is measured in one or more astrocytes in the putamen.

41. The method of claim 35, wherein the expression of the HTT gene product (RNA or protein) is inhibited or suppressed in the pyramidal neurons of each of the primary motor cortex, primary somatosensory cortex, and the temporal cortex.

42. The method of claim 35, wherein the expression of the HTT gene product (RNA or protein is reduced by at least 20% in the cerebral cortex.

43. The method of claim 35, wherein the expression of the HTT gene product (RNA or protein) is inhibited or suppressed in both the striatum and the cerebral cortex of the patient.

44. The method of claim 35 wherein the HTT gene product is the HTT protein and the HTT protein expression is inhibited or suppressed in the striatum, putamen, caudate and/or thalamus of the patient.

45. The method of claim 44, wherein the level of the HTT protein is reduced by at least 10% in the putamen.

46. The method of claim 45, wherein the level of the HTT protein is reduced by 15-65% in the putamen.

47. The method of claim 44, wherein e level of the HTT protein is reduced by at least 5% in the caudate.

48. The method of claim 47, wherein the level of the HTT protein is reduced by 5-50% in the caudate.

49. The method of claim 44, wherein the level of the HTT protein is reduced by at least 10% in the thalamus.

50. The method of claim 44, wherein the level of the HTT protein is reduced by 15-80% in the thalamus.

51. The method of claim 44, wherein the level of the HTT protein is reduced in both the striatum and the thalamus of the patient.
